(19) United States
(12) Patent Application Publication
(10) Pub. No.: US 2022/0333208 A1
(43) Pub. Date: Oct. 20, 2022

(54) CRISPR EFFECTOR SYSTEM BASED MULTIPLEX CANCER DIAGNOSTICS

(71) Applicants: THE BROAD INSTITUTE, INC., Cambridge, MA (US); MASSACHUSETTS INSTITUTE OF TECHNOLOGY, Cambridge, MA (US); DANA-FARBER CANCER INSTITUTE, INC., Boston, MA (US)

(72) Inventors: Jonathan Gootenberg, Cambridge, MA (US); Omar Abudayyeh, Cambridge, MA (US); Jeremy Koob, Cambridge, MA (US); Rahul Vedula, Boston, MA (US); Coleman Lindsley, Boston, MA (US); Feng Zhang, Cambridge, MA (US)

(73) Assignees: THE BROAD INSTITUTE, INC., Cambridge, MA (US); MASSACHUSETTS INSTITUTE OF TECHNOLOGY, Cambridge, MA (US); DANA-FARBER CANCER INSTITUTE, INC., Boston, MA (US)

(21) Appl. No.: 17/640,016

(22) PCT Filed: Sep. 3, 2020
(86) PCT No.: PCT/US2020/049257

§ 371 Date: Mar. 3, 2022

Related U.S. Application Data
(60) Provisional application No. 62/895,415, filed on Sep. 3, 2019.

(51) Int. Cl. C12Q 1/6886; C12Q 1/682

(52) U.S. Cl. CPC C12Q 1/6886; C12Q 1/682; C12Q 2600/16

(57) ABSTRACT

Systems and methods for rapid diagnostics related to the use of CRISPR effector systems and optimized guide sequences, including multiplex lateral flow diagnostic devices and methods of use, including for detection of cancer markers, are provided.

### CROSS-REFERENCE TO RELATED APPLICATIONS

This application claims the benefit of U.S. Provisional Application No. 62/895,415, filed Sep. 3, 2019. The entire contents of the above-identified applications are hereby fully incorporated herein by reference.

### STATEMENT REGARDING FEDERALLY SPONSORED RESEARCH

This invention was made with government support under Grant No.(s) MH110049, HL141201, HG009761, and CA210382, awarded by the National Institutes of Health. The government has certain rights in the invention.

### REFERENCE TO AN ELECTRONIC SEQUENCE LISTING

The contents of the electronic sequence listing (“BROD-4630WP_ST25.txt”; Size is 659,305 bytes (659 KB on disk) and it was created on Sep. 3, 2020) is herein incorporated by reference in its entirety.

### TECHNICAL FIELD

The subject matter disclosed herein is generally directed to rapid diagnostics related to the use of CRISPR detection systems, in particular cancer diagnostics.

### BACKGROUND

Nucleic acids are a universal signature of biological information. The ability to rapidly detect nucleic acids with high sensitivity and single-base specificity on a portable platform has the potential to revolutionize diagnosis and monitoring for many diseases, provide valuable epidemiological information, and serve as a generalizable scientific tool. Although many methods have been developed for detecting nucleic acids (Du et al., 2017; Green et al., 2014; Kumar et al., 2014; Pardee et al., 2014; Pardee et al., 2016; Urdea et al., 2006), they inevitably suffer from trade-offs among sensitivity, specificity, simplicity, and speed. For example, qPCR approaches are sensitive but are expensive and rely on complex instrumentation, limiting usability to highly trained operators in laboratory settings. Other approaches, such as new methods combining isothermal nucleic acid amplification with portable platforms (Du et al., 2017; Pardee et al., 2016), offer high detection specificity in a point-of-care (POC) setting, but have somewhat limited applications due to low sensitivity. As nucleic acid diagnostics become increasingly relevant for a variety of healthcare applications, detection technologies that provide high specificity and sensitivity at low cost would be of great utility in both clinical and basic research settings.

Sensitive and rapid detection of nucleic acids is important for clinical diagnostics and biotechnological applications. The development of data-driven models for aiding experimental design has featured prominently during the maturation of molecular tools. Software for choosing optimal primer or probe sequences is vital for amplification and molecular detection technologies as well as CRISPR-based methods. Genome-informed thermodynamic models for primer selection (Ye, 2012), computational probe design for nucleic acid detection (Kim, 2015), and machine learning models for CRISPR off-target (Hsu, 2013) and on-target (Doench, 2014) prediction have all broadened use of corresponding technologies. An accurate model for activity-based Cas13 guide selection would facilitate design of optimal SHERLOCK assays, especially in applications requiring high-activity guides like lateral flow detection, and enable guide RNA design for in vivo RNA targeting applications with Cas13. Particularly useful applications include rapid identification of diseases such as cancer where identification is critical for proper treatment and prognosis.

### SUMMARY

In certain example embodiments, a nucleic acid detection system is provided for detecting the presence of one or more cancers in a sample comprising one or more CRISPR system comprising one or more Cas polypeptides and one or more optimized guide molecules designed to bind to one or more corresponding target molecules of one or more cancer fusion genes; and one or more detection constructs.

In embodiments, the detection construct is an RNA-based detection construct, which can be a masking construct that suppresses generation of a detectable signal. In one aspect, the masking construct suppresses generation of a detectable positive signal by masking the detectable positive signal, or generating a detectable negative signal instead. The RNA-based masking construct can comprise a silencing RNA that suppresses generation of a gene product encoded by a reporting construct, wherein the gene product generates the detectable positive signal when expressed. In embodiments, the RNA-based masking construct is a ribozyme that generates the negative detectable signal, and wherein the positive detectable signal is generated when the ribozyme is deactivated. In an aspect, the ribozyme converts a substrate to a first color and wherein the substrate converts to a second color when the ribozyme is deactivated.

The RNA-based masking construct, in embodiments, is an RNA aptamer and/or comprises an RNA-tethered inhibitor. In an aspect, the aptamer or RNA-tethered inhibitor sequesters an enzyme, wherein the enzyme generates a detectable signal upon release from the aptamer or RNA tethered inhibitor by acting upon a substrate. The aptamer can be an inhibitory aptamer that inhibits an enzyme and prevents the enzyme from catalyzing generation of a detectable signal from a substrate or wherein the RNA-tethered inhibitor inhibits an enzyme and prevents the enzyme from catalyzing generation of a detectable signal from a substrate. In embodiments, the enzyme is thrombin, protein C, neutrophil elastase, subtilisin, horseradish peroxidase, beta-galactosidase, or calf alkaline phosphatase. The enzyme can be thrombin, in embodiments, and the substrate is para-nitroanilide covalently linked to a peptide substrate for thrombin, or 7-amino-4-methylcoumarin covalently linked to a peptide substrate for thrombin. In some embodiments, the aptamer sequesters a pair of agents that when released from the aptamers combine to generate a detectable signal.

In an aspect, the RNA-based masking construct comprises an RNA oligonucleotide to which a detectable ligand and a masking component are attached. The RNA-based masking construct can comprise a nanoparticle held in aggregate by bridge molecules, wherein at least a portion of the bridge molecules comprises RNA, and wherein the solution undergoes a color shift when the nanoparticle is disbursed in solution. In an aspect, the nanoparticle is a colloidal metal, optionally colloidal gold. In embodiments, the detection construct is a gold nanoparticle, optionally modified with a binding agent that specifically binds the second molecule of the detection construct.

Systems and methods disclosed herein may use an RNA-based masking construct comprising a quantum dot linked to one or more quencher molecules by a linking molecule, wherein at least a portion of the linking molecule comprises RNA. The RNA-based masking construct can comprise RNA in complex with an intercalating agent, wherein the intercalating agent changes absorbance upon cleavage of the RNA. In an aspect, the intercalating agent is pyronine-Y or methylene blue. Detectable ligands used herein can comprise a fluorophore with a masking component that is a quencher molecule. In embodiments, the RNA-based detection construct is a nucleic-acid based aptamer comprising quadruplex having enzymatic activity, which can be peroxidase activity in some embodiments.

The detection construct can comprise a first molecule on a first end and a second molecule on a second end. In embodiments, FAM is the first molecule and biotin or Digoxigenin (DIG) is the second molecule. In embodiments, Tye665 is the first molecule and Alexa-488 or FAM is the second molecule.

In certain embodiments, the systems and methods detect one or more cancers selected from acute promyelocytic leukemia (APML), chronic myeloid leukemia (CIVIL), and/or acute lymphoblastic leukemia (ALL). In an aspect, the PML-RARa fusion is the PML-RARa intron/exon 6 fusion, or the PML-RARa fusion is the PML-RARa intron 3 fusion. In certain embodiments, the Cas protein is LwaCas13a and the guide molecule comprises SEQ ID NO: 2761, 2764, 2767, 2770, 2773, 2776, 2779, 2782, 2785, 2788, 2791, 2794, 2797, 2800, 2803, 2806, 2809, 2812, 2815, 2818, 2821, 2824, 2827, 2830, 2833, 2836, 2839, 2842, 2845, 2848, 2851, 2854, 2857, 2860, 2863, 2866, 2869, 2872, 2875, 2878, 2881, 2884, or 2887. In certain embodiments, the Cas protein is LwaCas13a and the guide molecule comprises SEQ ID NO: 2760, 2763, 2766, 2769, 2772, 2775, 2778, 2781, 2784, 2787, 2790, 2793, 2796, 2799, 2802, 2805, 2808, 2811, 2814, 2817, 2820, 2823, 2826, 2829, 2832, 2835, 2838, 2841, 2844, 2847, 2850, 2853, 2856, 2859, 2862, 2865, 2868, 2871, 2874, 2877, 2880, 2883, 2886, 3189, or 3195. In certain embodiments, the Cas protein is CcaCas13b and the guide molecule comprises SEQ ID NO: 2890, 2893, 2896, 2899, 2902, 2905, 2908, 2911, 2914, 2917, 2920, 2923, 2926, 2929, 2932, 2935, 2938, 2941, 2944, 2947, 2950, 2953, 2956, 2959, 2962, 2965, 2968, 2971, 2974, 2977, 2980, 2983, 2986, 2989, 2992, 2995, 2998, or 3001. In certain embodiments, the Cas protein is CcaCas13b and the guide molecule comprises SEQ ID NO: 2889, 2892, 2895, 2898, 2901, 2904, 2907, 2910, 2913, 2916, 2919, 2922, 2925, 2928, 2931, 2934, 2937, 2940, 2943, 2946, 2949, 2952, 2955, 2958, 2961, 2964, 2967, 2970, 2973, 2976, 2979, 2982, 2985, 2988, 2991, 2994, 2997, 3171, 3207, 3177 or 3213.

In embodiments, the BCR-ABL fusion is the BCR-ABL p210 b3a2 fusion, b2a2 fusion, or a p190 ela2 fusion. In an aspect, the top guide, or optimized guide, is generated for a Cas13 ortholog, in an aspect, the optimized guide is generated for an LwaCas13a or a CcaCas13b ortholog In an aspect, the Cas protein is LwaCas13a and the guide molecule comprises a top predicted guide from SEQ ID NOs: 3153, 3159, 3189 or 3195. In certain embodiments, the Cas protein is CcaCas13b and the guide molecule comprises a top predicted guide selected from SEQ ID NO: 3171, 3177, 3207, or 3213.

The one or more Cas polypeptides in systems and methods disclosed herein include one or more Type V Cas proteins, one or more Type VI proteins, or a combination of Type V and Type VI proteins. In an aspect, the Type VI Cas protein is a Cas13. In an aspect, the Type V Cas polypeptide is a Cas12 polypeptide.

The optimized guide for the target molecule can, in one aspect, be generated by pooling a set of guides, the guides produced by tiling guides across the target molecule; incubating the set of guides with a Cas polypeptide and the target molecule and measuring cleavage activity of each guide in the set; creating a training model based on the cleavage activity of the set of guides in the incubating step; predicting highly active guides for the target molecule; and identifying the optimized guides by incubating the predicted highly active In an aspect, the training model comprises one or more input features selected from guide sequence, flanking target sequence, normalized positions of the guide in the target and guide GC content. In embodiments, the guide sequence and/or flanking sequence input comprises one hit encoding mono-nucleotide and/or dinucleotide based identities across a guide length and flanking sequence in the target. In an aspect, the training model comprises applying logistic regression model on the activity of the guides across the one or more input features. The step of predicting highly active guides for the target molecule can comprise selecting guides with an increase in activity of a guide relative to the median activity, or selecting guides with highest guide activity. In embodiments, the increase in activity is measured by an increase in fluorescence. In an aspect, the guides are selected with a 1.5, 2, 2.5 or 3-fold activity relative to median, or are in the top quartile or quintile for each target tested. Optimized guides can be generated for a Cas13 ortholog with the methods disclosed herein and for use in the systems presently disclosed. In an aspect, the optimized guide is generated for an LwaCas13a or a Cca13b ortholog.

One or more amplification reagents to amplify the one or more target molecules can be provided in certain embodiments. In an aspect, the reagents to amplify the one or more target RNA molecules comprise nucleic acid sequence-based amplification (NASBA), recombinase polymerase amplification (RPA), loop-mediated isothermal amplification (LAMP), strand displacement amplification (SDA), helicase-dependent amplification (HDA), nicking enzyme amplification reaction (NEAR), PCR, multiple displacement amplification (MDA), rolling circle amplification (RCA), ligase chain reaction (LCR), or ramification amplification method (RAM).

Lateral flow devices are provided comprising a substrate comprising a first end, wherein the first end comprises a sample loading portion and a first region loaded with a detection construct and one or more nucleic acid detection systems of any one of the preceding claims, a first capture region comprising a first binding agent, and a second capture region comprising a second binding agent. In embodiments, the later flow device sample loading portion further comprises one or more amplification reagents to amplify the one or more target molecules.

Methods for detecting a cancer fusion gene in a sample, comprising contacting the sample with the nucleic acid detection system as disclosed herein. Methods can comprise amplifying the target molecules in the sample by RT-RPA, optionally with AMV RT.

In one aspect, contacting the sample with the nucleic acid detection system comprises contacting the sample with a lateral flow device. The sample can, in some embodiments, be blood, bone marrow, or pelleted cells. In certain instances, where the sample comprises cells, the method comprises the step of lysing the pelleted cells. In an aspect, the method can comprise extracting RNA from a crude sample for detection.

The methods disclosed herein can further comprise steps of extracting RNA, performing RT-RPA, performing T7 transcription, and detecting the target nucleic acids. In one aspect, detecting the target nucleic acids comprises activating the Cas protein via binding of the one or more guide molecules to the one or more cancer-specific target molecules, wherein activating the Cas protein results in modification of the RNA-based masking construct such that a detectable positive signal is produced; and detecting the signal, wherein detection of the signal indicates the presence of a cancer-specific fusion gene. The detecting step can be s less than about 45 minutes to less than about 3 hours.

The present disclosure provides systems and methods wherein a plurality of cancer fusion genes can be detecting simultaneously on a multiplex lateral flow strip. In certain embodiments, detecting PML-RARa Intron/exon 6 fusion and Intron 3 fusion is performed simultaneously on multiplex lateral flow, which can optionally comprise a FAM and/or Alexa 488 molecules. In an aspect, the methods and systems can detect to a sensitivity of about 2 fM, or about 200 aM.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—SHERLOCK guide design machine learning model is capable of predicting highly active crRNAs for SHERLOCK detection. FIG. 1A Schematic of computational workflow of the SHERLOCK guide design tool; FIG. 1B Collateral activity of LwaCas13a and Capnocytophaga canimorsus Cc5 (CcaCas13b) with crRNAs tiling Ebola and Zika synthetic ssRNA targets; FIG. 1C ROC and AUC results of the best performing logistic regression model for LwaCas13 a (light gray) and CcaCas13b (dark gray) trained using crRNAs tiled and five different synthetic RNA targets; FIG. 1D Selected mono-nucleotide feature weights of the best performing logistic regression model for LwaCas13a (left) and CcaCas13b (right). Known PFS constraints are shown as letters above the appropriate flanking positions.

FIG. 2A-2E—SHERLOCK guide design machine learning model validates across many crRNAs, can predict crRNAs with high activity on lateral flow strips, and correlates with in vivo knockdown. FIG. 2A Validation of best performing model for LwaCas13a across multiple crRNAs, showing the predicted score of each crRNA versus actual collateral activity upon target recognition of thermonuclease, APML long, or APML short synthetic targets. The best and worst crRNAs predicted by the model are highlighted in blue and red, respectively. FIG. 2B Kinetic data of predicted best and worst performing LwaCas13 a crRNAs highlighted in panel 2a on thermonuclease, APML long, and APML short synthetic RNA targets. FIG. 2C Lateral flow performance of the predicted best and worst LwaCas13a crRNAs from panel 2a on detecting thermonuclease, APML long, and APML short synthetic RNA targets. FIG. 2D Schematic for evaluating the predictive performance of the guide design model for in vivo knockdown activity. FIG. 2E Previously measured knockdown activity of LwaCas13 a guides tiled across Gluc and KRAS targets¹⁴ was ranked according to the predicted activity of the guide based on the guide design model. The means of the distributions are shown as red dotted lines while the quartiles are shown as blue dotted lines. ***p<0.001; *p<0.05; two-tailed student's T-test.

FIG. 3A-3L—One-pot RPA-SHERLOCK is capable of rapid and portable detection of different targets. FIG. 3A Schematic of one-pot LwaCas13a SHERLOCK detection of acyltransferase target from P. aeruginosa with the best and worst predicted crRNAs from the guide design model; FIG. 3B Kinetic curves of one-pot LwaCas13a SHERLOCK detection of acyltransferase target from P. aeruginosa with the best predicted crRNA; FIG. 3C Kinetic curves of one-pot LwaCas13a SHERLOCK detection of acyltransferase target from P. aeruginosa with the worst predicted crRNA; FIG. 3D One-pot LwaCas13a SHERLOCK end-point detection of acyltransferase target from P. aeruginosa for the best and worst crRNAs at 1 hour; FIG. 3E One-pot LwaCas13a SHERLOCK lateral flow detection of acyltransferase target from P. aeruginosa using the best and worst predicted crRNAs at 1 hour; FIG. 3F Quantitation of one-pot LwaCas13a SHERLOCK end-point lateral flow detection of acyltransferase target from P. aeruginosa using the best and worst predicted crRNAs at 1 hour; FIG. 3G Schematic CcaCas13b one-pot SHERLOCK detection of thermonuclease target from S. aureus with the best and worst predicted crRNAs from the guide design model; FIG. 3H Kinetic curves of one-pot CcaCas13b SHERLOCK detection of thermonuclease target from S. aureus with the best predicted crRNA; FIG. 3I Kinetic curves of one-pot CcaCas13b SHERLOCK detection of thermonuclease target from S. aureus with the worst predicted crRNA; FIG. 3J One-pot CcaCas13b SHERLOCK end-point detection of thermonuclease target from S. aureus for the best and worst crRNAs at 1 hour; FIG. 3K One-pot CcaCas13b SHERLOCK lateral flow detection of thermonuclease target from S. aureus using the best and worst predicted crRNAs at 1 hour; FIG. 3L Quantitation of one-pot CcaCas13b SHERLOCK end-point lateral flow detection of thermonuclease target from S. aureus using the best and worst predicted crRNAs at 1 hour.

FIG. 4A-4E Multiplexed lateral flow detection with SHERLOCK. FIG. 4A Schematic of multiplex detection with one-pot SHERLOCK, with either fluorescent readout or lateral flow format. FIG. 4B Multiplexed fluorescence detection with one-pot SHERLOCK detection of Ea175 and thermonuclease targets using LwaCas13a and CcaCas13b orthologs, respectively, and the best predicted cRNAs; FIG. 4C Schematic of multiplex lateral flow with SHERLOCK; FIG. 4D Representative images of multiplexed lateral flow detection with one-pot SHERLOCK of Ea175 and thermonuclease targets using LwaCas13a and CcaCas13b orthologs, with quantitation of lateral flow strip band intensities. Lateral flow strip band intensities are inverted such that loss of signal is shown as positive signal; FIG. 4E Multiplexed lateral flow detection with one-pot SHERLOCK detection of Ea175 and thermonuclease targets using LwaCas13a and CcaCas13b orthologs, respectively, and the best predicted cRNAs. Lateral flow strip band intensities are inverted such that loss of signal is shown as positive signal.

FIG. 5A-5F Detection of PML-RARa and BCR-ABL cancer fusion transcripts from clinical samples. FIG. 5A Diagram of guide design for PML-RARa and BCR-ABL fusion transcripts tested in this study using the guide design model. Diagram of fusion transcripts adapted from van Dongen et al²⁸. FIG. 5B Workflow for SHERLOCK testing of clinical samples of patients exhibiting PML-RARa and BCR-ABL fusion transcripts. Patient blood or bone marrow is extracted, pelleted, and RNA is purified from patient cells. Extracted RNA is then used as input into an RT-RPA reaction, the products of which are used as input for Cas13 detection; FIG. 5C RT-PCR of APML and BCR-ABL cancer variants from purified RNA. Composite image is made up of bands cut out from several gels running PCR products for the different transcripts (full gel images shown in FIG. 14A-14E). PCR products for the different fusions should have the following sizes: PML-RARa Intron 6 (214 bp); PML-RARa Intron 3: 289 bp; BCR-ABL p210 e14a2 (360 bp); BCR-ABL p210 e13a2 (285 bp); BCR-ABL p190 e1a2 (381 bp); FIG. 5D Two-step SHERLOCK end-point fluorescence detection of PML-RARa and BCR-ABL fusion transcripts using best predicted crRNAs at 45 minutes. RNA from each patient was amplified using primer sets for the three fusion transcripts shown, and Cas13 detection was setup with corresponding crRNAs. Greyed out bars (sample 15) indicate that data was not collected; FIG. 5E Two-step SHERLOCK lateral flow detection of PML-RARa and BCR-ABL fusion transcripts using best predicted crRNAs at 3 hours. Sample bands were cropped out from the lateral flow strips; full lateral flow images, containing both sample and control bands, are shown in FIG. 15. Greyed out boxes (sample 15) indicate that data was not collected; FIG. 5F Quantitation of the lateral flow data shown in (e). Greyed out bars (sample 15) indicate that data was not collected.

FIG. 6A-6C Multiplexed detection of PML-RARa and BCR-ABL cancer fusion transcripts from clinical samples FIG. 6A Schematic of two-step SHERLOCK multiplexed detection from RNA input; FIG. 6B Images of multiplexed lateral flow detection with two-step SHERLOCK detection of PML-RARa Intron/Exon 6 and Intron 3 fusion transcripts using LwaCas13a and CcaCas13b orthologs, respectively, and the best predicted cRNAs; FIG. 6C Quantitation of lateral flow strip band intensities; data are inverted such that loss of signal is shown as positive signal.

FIG. 7A-7C Training data and features of the SHERLOCK guide design model. FIG. 7A Collateral activity of LwaCas13a (blue) and CcaCas13b (red) with crRNAs tiling Ebola and Zika synthetic ssRNA targets; FIG. 7B Mono-nucleotide feature weights of the best performing logistic regression model for LwaCas13a (top) and CcaCas13b (bottom); FIG. 7C. Di-nucleotide feature weights of the best performing logistic regression model for LwaCas13a (left) and CcaCas13b (right).

FIG. 8A-8C: SHERLOCK guide design machine learning model validates across many crRNAs (CcaCas13b). FIG. 8A. Validation of best performing model for CcaCas13b across multiple crRNAs, showing the predicted score of each crRNA versus actual collateral activity upon target recognition of thermonuclease, APML long, or APML short synthetic targets. The best and worst crRNAs predicted by the model are highlighted in light gray or dark gray, respectively. FIG. 8B. Kinetic data of predicted best and worst performing CcaCas13b crRNAs highlighted in panel 8a on thermonuclease, APML long, and APML short synthetic RNA targets. FIG. 8C. Lateral flow performance of the predicted best and worst CcaCas13b crRNAs from panel 8a on detecting thermonuclease, APML long, and APML short synthetic RNA targets.

FIG. 9 LwaCas13a guide design model predicts highly active guides for in vivo knockdown. A panel of guides predicted to be highly active or not active, as well as random guides, are tested for knockdown of the Gluc transcript in HEK293FT cells. Each data point represents the mean of three biological replicates. The means of the distributions are shown as red dotted lines while the quartiles are shown as dotted lines.

FIG. 10A-10F Additional targets are easily detected via one-pot SHERLOCK with lateral flow. FIG. 10A Kinetic curves of one-pot LwaCas13a SHERLOCK detection of Ea175 target. FIG. 10B One-pot LwaCas13a SHERLOCK end-point detection of Ea175 target at 45 minutes. FIG. 10C Quantitation of one-pot LwaCas13a SHERLOCK end-point lateral flow detection of Ea175 target at 30 minutes. FIG. 10D Kinetic curves of one-pot LwaCas13a SHERLOCK detection of Ea81 target. FIG. 10E One-pot LwaCas13a SHERLOCK end-point detection of Ea81 target at 45 minutes. FIG. 1OF Quantitation of one-pot LwaCas13a SHERLOCK end-point lateral flow detection of Ea81 target at 3 hours.

FIG. 11A-11F One-pot HDA-SHERLOCK is capable of quantitative detection of different targets. FIG. 11A Schematic of helicase reporter for screening DNA unwinding activity; FIG. 11B Temperature sensitivity screen of different helicase orthologs with and without super-helicase mutations using the high-throughput fluorescent reporter; FIG. 11C Schematic of one-pot SHERLOCK with RPA or Super-HAD; FIG. 11D Kinetic curves of one-pot RPA detection of a restriction endonuclease gene fragment (Ea175) from T denticola; FIG. 11E Kinetic curves of one-pot HDA detection of Ea175; FIG. 11F Quantitative nature of HDA-SHERLOCK compared to one-pot RPA.

FIG. 12A-12F Multiplexed lateral flow detection with two-pot SHERLOCK. FIG. 12A Schematic of multiplex lateral flow with RPA preamplification design for two probes; FIG. 12B Multiplexed lateral flow detection with RPA preamplification of two targets, ssDNA 1 and a gene fragment of lectin from soybeans; FIG. 12C Multiplexed lateral flow detection with RPA preamplification of two targets, ssDNA 1 and lectin gene fragment, at a range of concentrations down to 2 aM; FIG. 12D Schematic for custom-made lateral flow strips enabling detection of three targets simultaneously with SHERLOCK; FIG. 12E Images of multiplexed lateral flow strips detecting three targets, ssDNA 1, Zika ssRNA, and Dengue ssRNA, in various combinations using LwaCas13a, CcaCas13b, and AsCas12a; FIG. 12F Quantitation of Tye-665 fluorescent intensity of multiplexed lateral flow strips detecting three targets, ssDNA 1, Zika ssRNA, and Dengue ssRNA, in various combinations using LwaCas13 a, CcaCas13b, and AsCas12a.

FIG. 13A-13D SHERLOCK guide design machine learning model validates for crRNAs targeting BCR-ABL p210 b3a2. FIG. 13A Validation of best performing model for CcaCas13b across crRNAs tiling the BCR-ABL p210 b3a2 fusion transcript, showing the predicted score of each crRNA versus actual collateral activity upon target recognition. The best and worst crRNAs predicted by the model are highlighted in light gray or dark gray, respectively. FIG. 13B Validation of best performing model for LwaCas13a across crRNAs tiling the BCR-ABL p210 b3a2 fusion transcript, showing the predicted score of each crRNA versus actual collateral activity upon target recognition. The best and worst crRNAs predicted by the model are highlighted in blue or red, respectively. FIG. 13C Kinetic data of predicted best and worst performing LwaCas13a crRNAs highlighted in 13A on the BCR-ABL p210 b3a2 fusion transcript. FIG. 13D Kinetic data of predicted best and worst performing CcaCas13b crRNAs highlighted in 13B on the BCR-ABL p210 b3a2 fusion transcript.

FIG. 14A-14E Nested RT-PCR detection of PML-RARa and BCR-ABL cancer fusion transcripts from clinical samples. FIG. 14A Whole gel images of detection of PML-RARa Intron 6: 214 bp. For sample 6, because the breakpoint is in exon 6 of PML, the band size can be variable. FIG. 14B Whole gel images of detection of PML-RARa Intron 3: 289 bp. Some patients that have intron/exon 6 breakpoints, as in samples 4-6, can demonstrate several larger size bands (as seen), due to alternative splicing of PML. FIG. 14C Whole gel images of detection of BCR-ABL p210: e14a2 360 bp, e13a2 285 bp. FIG. 14D Whole gel images of detection of BCR-ABL p190: e1a2 381 bp. FIG. 14E Whole gel images of detection of GAPDH: 138 bp.

FIG. 15 Detection of PML-RARa and BCR-ABL cancer fusion transcripts from clinical samples. Two-step SHERLOCK lateral flow detection of PML-RARa and BCR-ABL fusion transcripts using best predicted crRNAs at 3 hours. Lateral flow strips are depicted with both the sample and control bands. Greyed out strips (sample 15) indicate that data was not collected.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011)

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to U.S. Provisional Application 62/181,663, U.S. Provisional Application 62/245,264, International Patent Publication WO2016/205749, International Patent Publication WO2016/205764, International Patent Publication WO2017/219027, International Patent Publication WO2018/107129, US Patent Publication 20180298445, US Patent Publication 20180274017, International Patent Publication WO2018/180340, International Patent Publication WO2018/191750, International Patent Publication WO2019/051318, International Patent Application PCT/US2018/054472, International Patent Application PCT/US2018/066940, International Patent Application PCT/US2019/015726, International Patent Application PCT/US2019/039221, International Patent Application PCT/US2019/039195, and International Patent Application PCT/US2019/09167.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide systems of detection utilizing optimized guides, and methods of using the detection systems. The detection systems comprise CRISPR systems for target molecule detection. The optimized guides provide sensitive detection and/or rapid kinetics allowing visualization of a signal that can be used in portable detection and use in clinical applications.

Optimized guides are provided using a guide prediction model to design optimal guides for sensitive detection of chromosomal fusion rearrangements characteristic of acute promyelocytic leukemia (APML) and acute lymphoblastic leukemia (ALL) in a multiplexed lateral flow readout. The combination of predictive guide design tools with a one-pot SHERLOCK format and multiplexed lateral flow detection allows for rapid deployment of robust and portable SHERLOCK assays in the laboratory, clinic, and field.

Sensitive and rapid detection of nucleic acids is important for clinical diagnostics and biotechnological applications. A platform previously developed by Applicants for nucleic acid detection using CRISPR enzymes, called SHERLOCK (Specific High Sensitivity Enzymatic Reporter unLOCKing)^{1,2}, combines pre-amplification with the RNA-guided RNase Cas13³⁻⁷ and DNase Cas12^{8,9} for sensing of nucleic acids. Here, the platform was extended by applying machine learning to predict strongly active guides for rapid detection of bacterial nucleic acid targets in an optimized one-pot reaction with lateral flow readout, with the developed guide prediction model used to design optimal guides for sensitive detection of chromosomal fusion rearrangements characteristic of acute promyelocytic leukemia (APML) and acute lymphoblastic leukemia (ALL) in a multiplexed lateral flow readout. The combination of predictive guide design tools with a one-pot SHERLOCK format and multiplexed lateral flow detection allows for rapid deployment of robust and portable SHERLOCK assays in the laboratory, clinic, and field.

### Nucleic Acid Detection Systems

### CRISPR Systems

In general, a CRISPR-Cas or CRISPR system as used herein and in documents, such as International Patent Publication No. WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). When the CRISPR protein is a C2c2 protein, a tracrRNA is not required. C2c2 has been described in Abudayyeh et al. (2016) “C2c2 is a single-component programmable RNA-guided RNA-targeting CRISPR effector”; Science; DOI: 10.1126/science.aaf5573; and Shmakov et al. (2015) “Discovery and Functional Characterization of Diverse Class 2 CRISPR-Cas Systems”, Molecular Cell, DOI: dx.doi.org/10.1016/j.molce1.2015.10.008; which are incorporated herein in their entirety by reference. Cas13b has been described in Smargon et al. (2017) “Cas13b Is a Type VI-B CRISPR-Associated RNA-Guided RNases Differentially Regulated by Accessory Proteins Csx27 and Csx28,” Molecular Cell. 65, 1-13; dx.doi.org/10.1016/j.molce1.2016.12.023., which is incorporated herein in its entirety by reference.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

In a preferred embodiment, the CRISPR effector protein may recognize a 3′ PAM. In certain embodiments, the CRISPR effector protein may recognize a 3′ PAM which is 5′H, wherein H is A, C or U. In certain embodiments, the effector protein may be Leptotrichia shahii C2c2p, more preferably Leptotrichia shahii DSM 19757 C2c2, and the 3′ PAM is a 5′ H.

In the context of formation of a CRISPR complex, “target molecule” or “target sequence” or “target nucleic acid” refers to a molecule harboring a sequence, or a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell. A target sequence may comprise DNA polynucleotides.

As such, a CRISPR system may comprise RNA-targeting effector proteins. A CRISPR system may comprise DNA-targeting effector proteins. In some embodiments, a CRISPR system may comprise a combination of RNA- and DNA-targeting effector proteins, or effector proteins that target both RNA and DNA.

The nucleic acid molecule encoding a CRISPR effector protein, in particular C2c2, is advantageously codon optimized CRISPR effector protein. An example of a codon optimized sequence, is in this instance a sequence optimized for expression in eukaryotes, e.g., humans (i.e. being optimized for expression in humans), or for another eukaryote, animal or mammal as herein discussed; see, e.g., SaCas9 human codon optimized sequence in International Patent Publication No. WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a CRISPR effector protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, the methods as described herein may comprise providing a Cas transgenic cell, in particular a C2c2 transgenic cell, in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to International Patent Publication No. WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus.

In certain aspects the invention involves vectors, e.g. for delivering or introducing in a cell Cas and/or RNA capable of guiding Cas to a target locus (i.e. guide RNA), but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety. Thus, the embodiments disclosed herein may also comprise transgenic cells comprising the CRISPR effector system. In certain example embodiments, the transgenic cell may function as an individual discrete volume. In other words samples comprising a masking construct may be delivered to a cell, for example in a suitable delivery vesicle and if the target is present in the delivery vesicle the CRISPR effector is activated and a detectable signal generated.

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well established cloning protocols and the teachings in this disclosure one skilled in the art can readily practice the invention as to the RNA(s) for a suitable exemplary vector such as AAV, and a suitable promoter such as the U6 promoter. For example, the packaging limit of AAV is ˜4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector, is to express an array of promoter-RNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordjournals.org/content/34/7/e53. short and nature.com/mt/journal/v16/n9/abs/mt2008144a.html). In an advantageous embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters—especially as to the numbers of RNAs or guides discussed herein, without any undue experimentation.

The guide RNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the promoter is U6.

In some embodiments, one or more elements of a nucleic acid-targeting system is derived from a particular organism comprising an endogenous CRISPR RNA-targeting system. In certain example embodiments, the effector protein CRISPR RNA-targeting system comprises at least one HEPN domain, including but not limited to the HEPN domains described herein, HEPN domains known in the art, and domains recognized to be HEPN domains by comparison to consensus sequence motifs. Several such domains are provided herein. In one non-limiting example, a consensus sequence can be derived from the sequences of C2c2 or Cas13b orthologs provided herein. In certain example embodiments, the effector protein comprises a single HEPN domain. In certain other example embodiments, the effector protein comprises two HEPN domains.

In one example embodiment, the effector protein comprises one or more HEPN domains comprising a RxxxxH motif sequence. The RxxxxH motif sequence can be, without limitation, from a HEPN domain described herein or a HEPN domain known in the art. RxxxxH motif sequences further include motif sequences created by combining portions of two or more HEPN domains. As noted, consensus sequences can be derived from the sequences of the orthologs disclosed in U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional Patent Application entitled “Novel Type VI CRISPR Orthologs and Systems,” labeled as attorney docket number 47627-05-2133 and filed on Apr. 12, 2017.

In an embodiment of the invention, a HEPN domain comprises at least one RxxxxH motif comprising the sequence of R(N/H/K)X1X2X3H (SEQ ID NO:1-3). In an embodiment of the invention, a HEPN domain comprises a RxxxxH motif comprising the sequence of R(N/H)X1X2X3H (SEQ ID NO:145). In an embodiment of the invention, a HEPN domain comprises the sequence of R(N/K)X1X2X3H (SEQ ID NO:4-5). In certain embodiments, X1 is R, S, D, E, Q, N, G, Y, or H. In certain embodiments, X2 is I, S, T, V, or L. In certain embodiments, X3 is L, F, N, Y, V, I, S, D, E, or A.

Embodiments disclosed herein utilize Cas proteins possessing non-specific nuclease collateral activity to cleave detectable reporters upon target recognition, providing sensitive and specific diagnostics, including single nucleotide variants, detection based on rRNA sequences, screening for drug resistance, monitoring microbe outbreaks, genetic perturbations, and screening of environmental samples, as described, for example, in PCT/US18/054472 filed Oct. 22, 2018 at [0183]-[0327], incorporated herein by reference. Reference is made to WO 2017/219027, WO2018/107129, US20180298445, US 2018-0274017, US 2018-0305773, WO 2018/170340, U.S. application Ser. No. 15/922,837, filed Mar. 15, 2018 entitled “Devices for CRISPR Effector System Based Diagnostics”, PCT/US18/50091, filed Sep. 7, 2018 “Multi-Effector CRISPR Based Diagnostic Systems”, PCT/US18/66940 filed Dec. 20, 2018 entitled “CRISPR Effector System Based Multiplex Diagnostics”, PCT/US18/054472 filed Oct. 4, 2018 entitled “CRISPR Effector System Based Diagnostic”, U.S. Provisional 62/740,728 filed Oct. 3, 2018 entitled “CRISPR Effector System Based Diagnostics for Hemorrhagic Fever Detection”, U.S. Provisional 62/690,278 filed Jun. 26, 2018 and U.S. Provisional 62/767,059 filed Nov. 14, 2018 both entitled “CRISPR Double Nickase Based Amplification, Compositions, Systems and Methods”, U.S. Provisional 62/690,160 filed Jun. 26, 2018 and 62,767,077 filed Nov. 14, 2018, both entitled “CRISPR/CAS and Transposase Based Amplification Compositions, Systems, And Methods”, U.S. Provisional 62/690,257 filed Jun. 26, 2018 and 62/767,052 filed Nov. 14, 2018 both entitled “CRISPR Effector System Based Amplification Methods, Systems, And Diagnostics”, U.S. Provisional 62/767,076 filed Nov. 14, 2018 entitled “Multiplexing Highly Evolving Viral Variants With SHERLOCK” and 62/767,070 filed Nov. 14, 2018 entitled “Droplet SHERLOCK.” Reference is further made to WO2017/127807, WO2017/184786, WO 2017/184768, WO 2017/189308, WO 2018/035388, WO 2018/170333, WO 2018/191388, WO 2018/213708, WO 2019/005866, PCT/US18/67328 filed December 21, 2018 entitled “Novel CRISPR Enzymes and Systems”, PCT/US18/67225 filed Dec. 21, 2018 entitled “Novel CRISPR Enzymes and Systems” and PCT/US18/67307 filed Dec. 21, 2018 entitled “Novel CRISPR Enzymes and Systems”, U.S. 62/712,809 filed Jul. 31, 2018 entitled “Novel CRISPR Enzymes and Systems”, U.S. 62/744,080 filed Oct. 10, 2018 entitled “Novel Cas12b Enzymes and Systems” and U.S. 62/751,196 filed Oct. 26 2018 entitled “Novel Cas12b Enzymes and Systems”, U.S. Pat. No. 715,640 filed August 7, 2-18 entitled “Novel CRISPR Enzymes and Systems”, WO 2016/205711, U.S. Pat. No. 9,790,490, WO 2016/205749, WO 2016/205764, WO 2017/070605, WO 2017/106657, and WO 2016/149661, WO2018/035387, WO2018/194963, Cox DBT, et al., RNA editing with CRISPR-Cas13, Science. 2017 Nov. 24; 358(6366):1019-1027; Gootenberg J S, et al., Multiplexed and portable nucleic acid detection platform with Cas13, Cas12a, and Csm6., Science. 2018 Apr. 27; 360(6387):439-444; Gootenberg J S, et al., Nucleic acid detection with CRISPR-Cas13a/C2c2., Science. 2017 Apr. 28; 356(6336):438-442; Abudayyeh OO, et al., RNA targeting with CRISPR-Cas13, Nature. 2017 Oct. 12; 550(7675):280-284; Smargon A A, et al., Cas13b Is a Type VI-B CRISPR-Associated RNA-Guided RNase Differentially Regulated by Accessory Proteins Csx27 and Csx28. Mol Cell. 2017 Feb. 16; 65(4):618-630.e7; Abudayyeh O, et al., C2c2 is a single-component programmable RNA-guided RNA-targeting CRISPR effector, Science. 2016 Aug. 5; 353(6299):aaf5573; Yang L, et al., Engineering and optimising deaminase fusions for genome editing. Nat Commun. 2016 Nov. 2; 7:13330, Myhrvold et al., Field deployable viral diagnostics using CRISPR-Cas13, Science 2018 360, 444-448, Shmakov et al. “Diversity and evolution of class 2 CRISPR-Cas systems,” Nat Rev Microbiol. 2017 15(3):169-182, each of which is incorporated herein by reference in its entirety.

When using two or more CRISPR effector systems, the CRISPR effector systems may be RNA-targeting effector proteins, DNA-targeting effector proteins, or a combination thereof. The RNA-targeting effector proteins may be a Type VI Cas protein, such as Cas13 protein, including Cas13b, Cas13c, or Cas13d. The DNA-targeting effector protein may be a Type V Cas protein, such as Cas12a (Cpf1), Cas12b (C2c2), Cas12c (C2c3), Cas X, Cas Y, or Cas14.

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j .molce1.2015.10.008.

RNA targeting Cas protein

In an aspect, the invention utilizes an RNA targeting Cas protein. In certain embodiments, protospacer flanking site, or protospacer flanking sequence (PFS) directs binding of the effector proteins (.e.g Type VI) as disclosed herein to the target locus of interest. A PFS is a region that can affect the efficacy of Cas13a mediated targeting, and may be adjacent to the protospacer target in certain Cas13a proteins, while other orthologs do not require a specific PFS.In a preferred embodiment, the CRISPR effector protein may recognize a 3′ PFS. In certain embodiments, the CRISPR effector protein may recognize a 3′ PFS which is 5′H, wherein H is A, C or U. See, e.g. Abudayyeh, 2016. In certain embodiments, the effector protein may be Leptotrichia shahii Cas13p, more preferably Leptotrichia shahii DSM 19757 Cas13, and the 3′ PFS is a 5′ H. In an aspect, design of guides can utilize the known PFS preferences of enzymes, for example, 3′ H for LwaCas13a and 5′-D/3′-NAA for CcaCas3b.

In the context of formation of a CRISPR complex, “target molecule” or “target sequence” or “target nucleic acid” refers to a molecule harboring a sequence, or a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell. A target sequence may comprise DNA polynucleotides.

As such, a CRISPR system may comprise RNA-targeting effector proteins. A CRISPR system may comprise DNA-targeting effector proteins. In some embodiments, a CRISPR system may comprise a combination of RNA- and DNA-targeting effector proteins, or effector proteins that target both RNA and DNA.

In some embodiments, one or more elements of a nucleic acid-targeting system is derived from a particular organism comprising an endogenous CRISPR RNA-targeting system. In certain example embodiments, the effector protein CRISPR RNA-targeting system comprises at least one HEPN domain, including but not limited to the HEPN domains described herein, HEPN domains known in the art, and domains recognized to be HEPN domains by comparison to consensus sequence motifs. Several such domains are provided herein. In one non-limiting example, a consensus sequence can be derived from the sequences of Cas13a or Cas13b orthologs provided herein. In certain example embodiments, the effector protein comprises a single HEPN domain. In certain other example embodiments, the effector protein comprises two HEPN domains.

In one example embodiment, the effector protein comprises one or more HEPN domains comprising a RxxxxH motif sequence. The RxxxxH motif sequence can be, without limitation, from a HEPN domain described herein or a HEPN domain known in the art. RxxxxH motif sequences further include motif sequences created by combining portions of two or more HEPN domains. As noted, consensus sequences can be derived from the sequences of the orthologs disclosed in U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional Patent Application entitled “Novel Type VI CRISPR Orthologs and Systems,” labeled as attorney docket number 47627-05-2133 and filed on Apr. 12, 2017.

In an embodiment of the invention, a HEPN domain comprises at least one RxxxxH motif comprising the sequence of R(N/H/K)X1X2X3H (SEQ ID NO:XX). In an embodiment of the invention, a HEPN domain comprises a RxxxxH motif comprising the sequence of R(N/H)X1X2X3H (SEQ ID NO:XX). In an embodiment of the invention, a HEPN domain comprises the sequence of R(N/K)X1X2X3H (SEQ ID NO:XX). In certain embodiments, X1 is R, S, D, E, Q, N, G, Y, or H. In certain embodiments, X2 is I, S, T, V, or L. In certain embodiments, X3 is L, F, N, Y, V, I, S, D, E, or A.

In particular embodiments, the Type VI RNA-targeting Cas enzyme is Cas13a. In other example embodiments, the Type VI RNA-targeting Cas enzyme is Cas13b. In certain embodiments, the Cas13b protein is from an organism of a genus selected from the group consisting of: Bergeyella, Prevotella, Porphyromonas, Bacterioides, Alistipes, Riemerella, Myroides, Capnocytophaga, Porphyromonas, Flavobacterium, Porphyromonas, Chryseobacterium, Paludibacter, Psychroflexus, Riemerella, Phaeodactylibacter, Sinomicrobium, Reichenbachiella.

In particular embodiments, the homologue or orthologue of a Type VI protein such as Cas13a as referred to herein has a sequence homology or identity of at least 30%, or at least 40%, or at least 50%, or at least 60%, or at least 70%, or at least 80%, more preferably at least 85%, even more preferably at least 90%, such as for instance at least 95% with a Type VI protein such as Cas13a (e.g., based on the wild-type sequence of any of Leptotrichia shahii Cas13a, Lachnospiraceae bacterium MA2020 Cas13a, Lachnospiraceae bacterium NK4A179 Cas13a, Clostridium aminophilum (DSM 10710) Cas13a, Carnobacterium gallinarum (DSM 4847) Cas13, Paludibacter propionicigenes (WB4) Cas13, Listeria weihenstephanensis (FSL R9-0317) Cas13, Listeriaceae bacterium (FSL M6-0635) Cas13, Listeria newyorkensis (FSL M6-0635) Cas13, Leptotrichia wadei (F0279) Cas13, Rhodobacter capsulatus (SB 1003) Cas13, Rhodobacter capsulatus (R121) Cas13, Rhodobacter capsulatus (DE442) Cas13, Leptotrichia wadei (Lw2) Cas13, or Listeria seeligeri Cas13). In further embodiments, the homologue or orthologue of a Type VI protein such as Cas13 as referred to herein has a sequence identity of at least 30%, or at least 40%, or at least 50%, or at least 60%, or at least 70%, or at least 80%, more preferably at least 85%, even more preferably at least 90%, such as for instance at least 95% with the wild type Cas13 (e.g., based on the wild-type sequence of any of Leptotrichia shahii Cas13, Lachnospiraceae bacterium MA2020 Cas13, Lachnospiraceae bacterium NK4A179 Cas13, Clostridium aminophilum (DSM 10710) Cas13, Carnobacterium gallinarum (DSM 4847) Cas13, Paludibacter propionicigenes (WB4) Cas13, Listeria weihenstephanensis (FSL R9-0317) Cas13, Listeriaceae bacterium (FSL M6-0635) Cas13, Listeria newyorkensis (FSL M6-0635) Cas13, Leptotrichia wadei (F0279) Cas13, Rhodobacter capsulatus (SB 1003) Cas13, Rhodobacter capsulatus (R121) Cas13, Rhodobacter capsulatus (DE442) Cas13, Leptotrichia wadei (Lw2) Cas13, or Listeria seeligeri Cas13).

In certain other example embodiments, the CRISPR system the effector protein is a Cas13 nuclease. The activity of Cas13 may depend on the presence of two HEPN domains. These have been shown to be RNase domains, i.e. nuclease (in particular an endonuclease) cutting RNA. Cas13a HEPN may also target DNA, or potentially DNA and/or RNA. On the basis that the HEPN domains of Cas13a are at least capable of binding to and, in their wild-type form, cutting RNA, then it is preferred that the Cas13a effector protein has RNase function. Regarding Cas13a CRISPR systems, reference is made to U.S. Provisional 62/351,662 filed on Jun. 17, 2016 and U.S. Provisional 62/376,377 filed on Aug. 17, 2016. Reference is also made to U.S. Provisional 62/351,803 filed on Jun. 17, 2016. Reference is also made to U.S. Provisional entitled “Novel Crispr Enzymes and Systems” filed Dec. 8, 2016 bearing Broad Institute No. 10035.PA4 and Attorney Docket No. 47627.03.2133. Reference is further made to East-Seletsky et al. “Two distinct RNase activities of CRISPR-C2c2 enable guide-RNA processing and RNA detection” Nature doi:10/1038/nature19802 and Abudayyeh et al. “C2c2 is a single-component programmable RNA-guided RNA targeting CRISPR effector” bioRxiv doi:10.1101/054742.

RNase function in CRISPR systems is known, for example mRNA targeting has been reported for certain type III CRISPR-Cas systems (Hale et al., 2014, Genes Dev, vol. 28, 2432-2443; Hale et al., 2009, Cell, vol. 139, 945-956; Peng et al., 2015, Nucleic acids research, vol. 43, 406-417) and provides significant advantages. In the Staphylococcus epidermis type III-A system, transcription across targets results in cleavage of the target DNA and its transcripts, mediated by independent active sites within the Cas10-Csm ribonucleoprotein effector protein complex (see, Samai et al., 2015, Cell, vol. 151, 1164-1174). A CRISPR-Cas system, composition or method targeting RNA via the present effector proteins is thus provided.

In an embodiment, the Cas protein may be a Cas13a ortholog of an organism of a genus which includes but is not limited to Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter. Species of organism of such a genus can be as otherwise herein discussed.

It will be appreciated that any of the functionalities described herein may be engineered into CRISPR enzymes from other orthologs, including chimeric enzymes comprising fragments from multiple orthologs. Examples of such orthologs are described elsewhere herein. Thus, chimeric enzymes may comprise fragments of CRISPR enzyme orthologs of an organism which includes but is not limited to Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter. A chimeric enzyme can comprise a first fragment and a second fragment, and the fragments can be of CRISPR enzyme orthologs of organisms of genera herein mentioned or of species herein mentioned; advantageously the fragments are from CRISPR enzyme orthologs of different species.

In embodiments, the Cas13a protein as referred to herein also encompasses a functional variant of Cas13a or a homologue or an orthologue thereof. A “functional variant” of a protein as used herein refers to a variant of such protein which retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide or peptide. Functional variants may be naturally occurring or may be man-made. Advantageous embodiments can involve engineered or non-naturally occurring Type VI RNA-targeting effector protein.

In an embodiment, nucleic acid molecule(s) encoding the Cas13 or an ortholog or homolog thereof, may be codon-optimized for expression in a eukaryotic cell. A eukaryote can be as herein discussed. Nucleic acid molecule(s) can be engineered or non-naturally occurring.

In an embodiment, the Cas13a or an ortholog or homolog thereof, may comprise one or more mutations (and hence nucleic acid molecule(s) coding for same may have mutation(s). The mutations may be artificially introduced mutations and may include but are not limited to one or more mutations in a catalytic domain. Examples of catalytic domains with reference to a Cas9 enzyme may include but are not limited to RuvC I, RuvC II, RuvC III and HNH domains.

In an embodiment, the Cas13a or an ortholog or homolog thereof, may comprise one or more mutations. The mutations may be artificially introduced mutations and may include but are not limited to one or more mutations in a catalytic domain. Examples of catalytic domains with reference to a Cas enzyme may include but are not limited to HEPN domains.

In an embodiment, the Cas13a or an ortholog or homolog thereof, may be used as a generic nucleic acid binding protein with fusion to or being operably linked to a functional domain. Exemplary functional domains may include but are not limited to translational initiator, translational activator, translational repressor, nucleases, in particular ribonucleases, a spliceosome, beads, a light inducible/controllable domain or a chemically inducible/controllable domain.

In certain example embodiments, the Cas13a effector protein may be from an organism selected from the group consisting of; Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, and Campylobacter.

In certain embodiments, the effector protein may be a Listeria sp. Cas13p, preferably Listeria seeligeria Cas13p, more preferably Listeria seeligeria serovar 1/2b str. SLCC3954 Cas13p and the crRNA sequence may be 44 to 47 nucleotides in length, with a 5′ 29-nt direct repeat (DR) and a 15-nt to 18-nt spacer.

In certain embodiments, the effector protein may be a Leptotrichia sp. Cas13p, preferably Leptotrichia shahii Cas13p, more preferably Leptotrichia shahii DSM 19757 Cas13p and the crRNA sequence may be 42 to 58 nucleotides in length, with a 5′ direct repeat of at least 24 nt, such as a 5′ 24-28-nt direct repeat (DR) and a spacer of at least 14 nt, such as a 14-nt to 28-nt spacer, or a spacer of at least 18 nt, such as 19, 20, 21, 22, or more nt, such as 18-28, 19-28, 20-28, 21-28, or 22-28 nt.

In certain example embodiments, the effector protein may be a Leptotrichia sp., Leptotrichia wadei F0279, or a Listeria sp., preferably Listeria newyorkensis FSL M6-0635.

In certain example embodiments, the Cas13 effector proteins of the invention include, without limitation, the following 21 ortholog species (including multiple CRISPR loci: Leptotrichia shahii; Leptotrichia wadei (Lw2); Listeria seeligeri; Lachnospiraceae bacterium MA2020; Lachnospiraceae bacterium NK4A179; [Clostridium] aminophilum DSM 10710; Carnobacterium gallinarum DSM 4847; Carnobacterium gallinarum DSM 4847 (second CRISPR Loci); Paludibacter propionicigenes WB4; Listeria weihenstephanensis FSL R9-0317; Listeriaceae bacterium FSL M6-0635; Leptotrichia wadei F0279; Rhodobacter capsulatus SB 1003; Rhodobacter capsulatus R121; Rhodobacter capsulatus DE442; Leptotrichia buccalis C-1013-b; Herbinix hemicellulosilytica; [Eubacterium] rectale; Eubacteriaceae bacterium CHKCI004; Blautia sp. Marseille-P2398; and Leptotrichia sp. oral taxon 879 str. F0557. Twelve (12) further non-limiting examples are: Lachnospiraceae bacterium NK4A144; Chloroflexus aggregans; Demequina aurantiaca; Thalassospira sp. TSL5-1; Pseudobutyrivibrio sp. OR37; Butyrivibrio sp. YAB3001; Blautia sp. Marseille-P2398; Leptotrichia sp. Marseille-P3007; Bacteroides ihuae; Porphyromonadaceae bacterium KH3CP3RA; Listeria riparia; and Insolitispirillum peregrinum.

In certain embodiments, the Cas13 protein according to the invention is or is derived from one of the orthologues as described herein, or is a chimeric protein of two or more of the orthologues as described herein, or is a mutant or variant of one of the orthologues as described in below (or a chimeric mutant or variant), including dead Cas13, split Cas13, destabilized Cas13, etc. as defined herein elsewhere, with or without fusion with a heterologous/functional domain.

In certain example embodiments, the Cas13a effector protein is from an organism of a genus selected from the group consisting of: Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Campylobacter, and Lachnospira.

In an embodiment of the invention, there is provided an effector protein which comprises an amino acid sequence having at least 80% sequence homology to the wild-type sequence of any of Leptotrichia shahii Cas13, Lachnospiraceae bacterium MA2020 Cas13, Lachnospiraceae bacterium NK4A179 Cas13, Clostridium aminophilum (DSM 10710) Cas13, Carnobacterium gallinarum (DSM 4847) Cas13, Paludibacter propionicigenes (WB4) Cas13, Listeria weihenstephanensis (FSL R9-0317) Cas13, Listeriaceae bacterium (FSL M6-0635) Cas13, Listeria newyorkensis (FSL M6-0635) Cas13, Leptotrichia wadei (F0279) Cas13, Rhodobacter capsulatus (SB 1003) Cas13, Rhodobacter capsulatus (R121) Cas13, Rhodobacter capsulatus (DE442) Cas13, Leptotrichia wadei (Lw2) Cas13, or Listeria seeligeri Cas13. According to the invention, a consensus sequence can be generated from multiple Cas13 orthologs, which can assist in locating conserved amino acid residues, and motifs, including but not limited to catalytic residues and HEPN motifs in Cas13 orthologs that mediate Cas13 function. One such consensus sequence, generated from selected orthologs.

In an embodiment of the invention, the effector protein comprises an amino acid sequence having at least 80% sequence homology to a Type VI effector protein consensus sequence including but not limited to a consensus sequence described herein.

In another non-limiting example, a sequence alignment tool to assist generation of a consensus sequence and identification of conserved residues is the MUSCLE alignment tool (www.ebi.ac.uk/Tools/msa/muscle/). For example, using MUSCLE, the following amino acid locations conserved among Cas13a orthologs can be identified in Leptotrichia wadei Cas13a:K2; K5; V6; E301; L331; 1335; N341; G351; K352; E375; L392; L396; D403; F446; I466; I470; R474 (HEPN); H475; H479 (HEPN), E508; P556; L561; 1595; Y596; F600; Y669; I673; F681; L685; Y761; L676; L779; Y782; L836; D847; Y863; L869; 1872; K879; I933; L954; I958; R961; Y965; E970; R971; D972; R1046 (HEPN), H1051 (HEPN), Y1075; D1076; K1078; K1080; I1083; I1090.

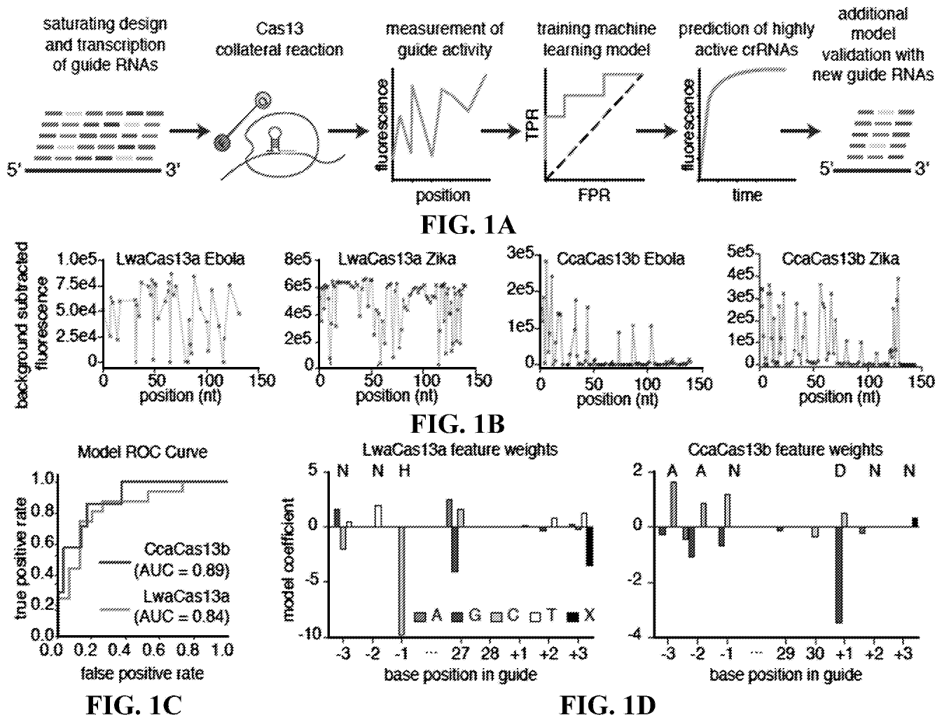

In certain example embodiments, the RNA-targeting effector protein is a Type VI-B effector protein, such as Cas13b and Group 29 or Group 30 proteins. In certain example embodiments, the RNA-targeting effector protein comprises one or more HEPN domains. In certain example embodiments, the RNA-targeting effector protein comprises a C-terminal HEPN domain, a N-terminal HEPN domain, or both. Regarding example Type VI-B effector proteins that may be used in the context of this invention, reference is made to U.S. application Ser. No. 15/331,792 entitled “Novel CRISPR Enzymes and Systems” and filed Oct. 21, 2016, International Patent Application No. PCT/US2016/058302 entitled “Novel CRISPR Enzymes and Systems”, and filed Oct. 21, 2016, and Smargon et al. “Cas13b is a Type VI-B CRISPR-associated RNA-Guided RNase differentially regulated by accessory proteins Csx27 and Csx28” Molecular Cell, 65, 1-13 (2017); dx.doi.org/10.1016/j.molce1.2016.12.023. In certain example embodiments, the Cas13b effector protein is, or comprises an amino acid sequence having at least 80% sequence homology to any of the sequences of Table 1 of International Patent Application No. PCT/US2016/058302. Further reference is made to example Type VI-B effector proteins of U.S. Provisional Application Nos. 62/471,710, 62/566,829 and International Patent Publication No. WO2018/1703333, entitled “Novel Cas13b Orthologues CRISPR Enzymes and System”. In particular embodiments, the Cas13b enzyme is derived from Bergeyella zoohelcum. In certain other example embodiments, the effector protein is, or comprises an amino acid sequence having at least 80% sequence homology to any of the sequences listed in Tables 1A or 1B of International Patent Publication No. WO2018/1703333, specifically incorporated herein by reference. In certain embodiments, the Cas 13b effector protein is, or comprises an amino acid sequence having at least 80% sequence homology to any of the polypeptides in U.S. Provisional Applications 62/484,791, 62/561,662, 62/568,129 or International Patent Publication WO2018/191388, all entitled “Novel Type VI CRISPR Orthologs and Systems,” incorporated herein by reference. In certain embodiments, the Cas13b effector protein is, or comprises an amin acid sequence having at least 80% sequence homology to a polypeptide as set forth in FIG. 1 of International Patent Publication WO2018/191388, specifically incorporated herein by reference. In an aspect, the Cas13b protein is selected from the group consisting of Porphyromonas gulae Cas13b (accession number WP 039434803), Prevotella sp. P5-125 Cas 13b (accession number WP 044065294), Porphyromonas gingivalis Cas 13b (accession number WP 053444417), Porphyromonas sp. COT-052 OH4946 Cas 13b (accession number WP 039428968), Bacteroides pyogenes Cas 13b (accession number WP 034542281), Riemerella anatipestifer Cas13b (accession number WP 004919755).

In certain example embodiments, the RNA-targeting effector protein is a Cas13c effector protein as disclosed in U.S. Provisional Patent Application No. 62/525,165 filed Jun. 26, 2017, and International Patent Publication No. WO2018/035250 filed Aug. 16, 2017. In certain example embodiments, the Cas13c protein may be from an organism of a genus such as Fusobacterium or Anaerosalibacter. Example wildtype orthologue sequences of Cas13c are: EH019081, WP 094899336, WP 040490876, WP 047396607, WP 035935671, WP 035906563, WP 042678931, WP 062627846, WP 005959231, WP 027128616, WP 062624740, WP 096402050.

In certain example embodiments, the Cas13 protein may be selected from any of the following: Cas13a: Leptotrichia shahii, Leptotrichia wadei (Lw2), Listeria seeligeri, Lachnospiraceae bacterium MA2020, Lachnospiraceae bacterium NK4A179, [Clostridium] aminophilum DSM 10710, Carnobacterium gallinarum DSM 4847, Carnobacterium gallinarum DSM 4847, Paludibacter propionicigenes WB4, Listeria weihenstephanensis FSL R9-0317, Listeriaceae bacterium FSL M6-0635, Leptotrichia wadei F0279, Rhodobacter capsulatus SB 1003, Rhodobacter capsulatus R121, Rhodobacter capsulatus DE442, Leptotrichia buccalis C-1013-b, Herbinix hemicellulosilytica, [Eubacterium] rectale, Eubacteriaceae bacterium CHKCI004, Blautia sp. Marseille-P2398, Leptotrichia sp. oral taxon 879 str. F0557; Cas 13b: Bergeyella zoohelcum, Prevotella intermedia, Prevotella buccae, Alistipes sp. ZOR0009, Prevotella sp. MA2016, Riemerella anatipestifer, Prevotella aurantiaca, Prevotella saccharolytica, Prevotella intermedia, Capnocytophaga canimorsus, Porphyromonas gulae, Prevotella sp. P5-125, Flavobacterium branchiophilum, Porphyromonas gingivalis, Prevotella intermedia; Cas13c: Fusobacterium necrophorum subsp. funduliforme ATCC 51357 contig00003, Fusobacterium necrophorum DJ-2 contig0065, whole genome shotgun sequence, Fusobacterium necrophorum BFTR-1 contig0068, Fusobacterium necrophorum subsp. funduliforme 1_1_36S cont1.14, Fusobacterium perfoetens ATCC 29250 T364DRAFT_scaffold00009.9_C, Fusobacterium ulcerans ATCC 49185 cont2.38, Anaerosalibacter sp. ND1 genome assembly Anaerosalibacter massiliensis ND1.Cas13s non-specific RNase activity can be leveraged to cleave reporters upon target recognition, allowing for the design of sensitive and specific diagnostics using Cas13, including single nucleotide variants, detection based on rRNA sequences, screening for drug resistance, monitoring microbe outbreaks, genetic perturbations, and screening of environmental samples, as described, for example, in PCT/US18/054472 filed Oct. 22, 2018 at [0183]-[0327], incorporated herein by reference. Reference is made to WO 2017/219027, WO2018/107129, US20180298445, US 2018-0274017, US 2018-0305773, WO 2018/170340, U.S. application Ser. No. 15/922,837, filed Mar. 15, 2018 entitled “Devices for CRISPR Effector System Based Diagnostics”, PCT/US18/50091, filed Sep. 7, 2018 “Multi-Effector CRISPR Based Diagnostic Systems”, PCT/US18/66940 filed Dec. 20, 2018 entitled “CRISPR Effector System Based Multiplex Diagnostics”, PCT/US18/054472 filed Oct. 4, 2018 entitled “CRISPR Effector System Based Diagnostic”, U.S. Provisional 62/740,728 filed Oct. 3, 2018 entitled “CRISPR Effector System Based Diagnostics for Hemorrhagic Fever Detection”, U.S. Provisional 62/690,278 filed Jun. 26, 2018 and U.S. Provisional 62/767,059 filed Nov. 14, 2018 both entitled “CRISPR Double Nickase Based Amplification, Compositions, Systems and Methods”, U.S. Provisional 62/690,160 filed Jun. 26, 2018 and 62,767,077 filed Nov. 14, 2018, both entitled “CRISPR/CAS and Transposase Based Amplification Compositions, Systems, And Methods”, U.S. Provisional 62/690,257 filed Jun. 26, 2018 and 62/767,052 filed Nov. 14, 2018 both entitled “CRISPR Effector System Based Amplification Methods, Systems, And Diagnostics”, U.S. Provisional 62/767,076 filed Nov. 14, 2018 entitled “Multiplexing Highly Evolving Viral Variants With SHERLOCK” and 62/767,070 filed Nov. 14, 2018 entitled “Droplet SHERLOCK.” Reference is further made to WO2017/127807, WO2017/184786, WO 2017/184768, WO 2017/189308, WO 2018/035388, WO 2018/170333, WO 2018/191388, WO 2018/213708, WO 2019/005866, PCT/US18/67328 filed Dec. 21, 2018 entitled “Novel CRISPR Enzymes and Systems”, PCT/US18/67225 filed Dec. 21, 2018 entitled “Novel CRISPR Enzymes and Systems”and PCT/US18/67307 filed Dec. 21, 2018 entitled “Novel CRISPR Enzymes and Systems”, U.S. 62/712,809 filed Jul. 31, 2018 entitled “Novel CRISPR Enzymes and Systems”, U.S. 62/744,080 filed Oct. 10, 2018 entitled “Novel Cas12b Enzymes and Systems” and U.S. 62/751,196 filed Oct. 26 2018 entitled “Novel Cas12b Enzymes and Systems”, U.S. Pat. No. 715,640 filed August 7, 2-18 entitled “Novel CRISPR Enzymes and Systems”, WO 2016/205711, U.S. Pat. No. 9,790,490, WO 2016/205749, WO 2016/205764, WO 2017/070605, WO 2017/106657, and WO 2016/149661, WO2018/035387, WO2018/194963, Cox DBT, et al., RNA editing with CRISPR-Cas13, Science. 2017 Nov. 24; 358(6366):1019-1027; Gootenberg J S, et al., Multiplexed and portable nucleic acid detection platform with Cas13, Cas12a, and Csm6., Science. 2018 Apr. 27; 360(6387):439-444; Gootenberg J S, et al., Nucleic acid detection with CRISPR-Cas13a/C2c2., Science. 2017 Apr. 28; 356(6336):438-442; Abudayyeh O O, et al., RNA targeting with CRISPR-Cas13, Nature. 2017 Oct. 12; 550(7675):280-284; Smargon A A, et al., Cas13b Is a Type VI-B CRISPR-Associated RNA-Guided RNase Differentially Regulated by Accessory Proteins Csx27 and Csx28. Mol Cell. 2017 Feb. 16; 65(4):618-630.e7; Abudayyeh O O, et al., C2c2 is a single-component programmable RNA-guided RNA-targeting CRISPR effector, Science. 2016 Aug 5; 353(6299):aaf5573; Yang L, et al., Engineering and optimising deaminase fusions for genome editing. Nat Commun. 2016 Nov. 2; 7:13330, Myrvhold et al., Field deployable viral diagnostics using CRISPR-Cas13, Science 2018 360, 444-448, Shmakov et al. “Diversity and evolution of class 2 CRISPR-Cas systems,” Nat Rev Microbiol. 2017 15(3):169-182, each of which is incorporated herein by reference in its entirety.

### DNA-Targeting Effector Proteins

In certain example embodiments, the assays may comprise a DNA-targeting effector protein. In certain example embodiments, the assays may comprise multiple DNA-targeting effectors or one or more orthologs in combination with one or more RNA-targeting effectors. In certain example embodiments, the DNA targeting are Type V Cas proteins, such as Cas12 proteins. In certain other example embodiments, the Cas12 proteins are Cas12a, Cas12b, Cas12c, or a combination thereof.

Cas 12a Orthologs

The present invention encompasses the use of a Cpf1 effector protein, derived from a Cpf1 locus denoted as subtype V-A. Herein such effector proteins are also referred to as “Cpf1p”, e.g., a Cpf1 protein (and such effector protein or Cpf1 protein or protein derived from a Cpf1 locus is also called “CRISPR enzyme”). Presently, the subtype V-A loci encompasses cas1, cas2, a distinct gene denoted cpfl and a CRISPR array. Cpf1 (CRISPR-associated protein Cpf1, subtype PREFRAN) is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in all Cas9 proteins, and the RuvC-like domain is contiguous in the Cpf1 sequence, in contrast to Cas9 where it contains long inserts including the HNH domain. Accordingly, in particular embodiments, the CRISPR-Cas enzyme comprises only a RuvC-like nuclease domain.

The programmability, specificity, and collateral activity of the RNA-guided Cpf1 also make it an ideal switchable nuclease for non-specific cleavage of nucleic acids. In one embodiment, a Cpf1 system is engineered to provide and take advantage of collateral non-specific cleavage of RNA. In another embodiment, a Cpf1 system is engineered to provide and take advantage of collateral non-specific cleavage of ssDNA. Accordingly, engineered Cpf1 systems provide platforms for nucleic acid detection and transcriptome manipulation. Cpf1 is developed for use as a mammalian transcript knockdown and binding tool. Cpf1 is capable of robust collateral cleavage of RNA and ssDNA when activated by sequence-specific targeted DNA binding.

Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April; 22(4):359-66. doi: 10.1002/pro.2225.). See also Shmakov et al. (2015) for application in the field of CRISPR-Cas loci. Homologous proteins may but need not be structurally related, or are only partially structurally related. The Cpf1 gene is found in several diverse bacterial genomes, typically in the same locus with cas1, cas2, and cas4 genes and a CRISPR cassette (for example, FNFX1_1431-FNFX1_1428 of Francisella cf. novicida Fx1). In particular embodiments, the effector protein is a Cpf1 effector protein from an organism from a genus comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus.

In further particular embodiments, the Cpf1 effector protein is from an organism selected from S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii.

The effector protein may comprise a chimeric effector protein comprising a first fragment from a first effector protein (e.g., a Cpf1) ortholog and a second fragment from a second effector (e.g., a Cpf1) protein ortholog, and wherein the first and second effector protein orthologs are different. At least one of the first and second effector protein (e.g., a Cpf1) orthologs may comprise an effector protein (e.g., a Cpf1) from an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus; e.g., a chimeric effector protein comprising a first fragment and a second fragment wherein each of the first and second fragments is selected from a Cpf1 of an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus wherein the first and second fragments are not from the same bacteria; for instance a chimeric effector protein comprising a first fragment and a second fragment wherein each of the first and second fragments is selected from a Cpf1 of S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii; Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae, wherein the first and second fragments are not from the same bacteria. In a more preferred embodiment, the Cpf1p is derived from a bacterial species selected from Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae. In certain embodiments, the Cpf1p is derived from a bacterial species selected from Acidaminococcus sp. BV3L6, Lachnospiraceae bacterium MA2020. In certain embodiments, the effector protein is derived from a subspecies of Francisella tularensis 1, including but not limited to Francisella tularensis subsp. Novicida.

In some embodiments, the Cpf1p is derived from an organism from the genus of Eubacterium. In some embodiments, the CRISPR effector protein is a Cpf1 protein derived from an organism from the bacterial species of Eubacterium rectale. In some embodiments, the amino acid sequence of the Cpf1 effector protein corresponds to NCBI Reference Sequence WP_055225123.1, NCBI Reference Sequence WP_055237260.1, NCBI Reference Sequence WP_055272206.1, or GenBank ID OLA16049.1. In some embodiments, the Cpf1 effector protein has a sequence homology or sequence identity of at least 60%, more particularly at least 70, such as at least 80%, more preferably at least 85%, even more preferably at least 90%, such as for instance at least 95%, with NCBI Reference Sequence WP_055225123.1, NCBI Reference Sequence WP_055237260.1, NCBI Reference Sequence WP_055272206.1, or GenBank ID OLA16049.1. The skilled person will understand that this includes truncated forms of the Cpf1 protein whereby the sequence identity is determined over the length of the truncated form. In some embodiments, the Cpf1 effector recognizes the PAM sequence of TTTN or CTTN.

In particular embodiments, the homologue or orthologue of Cpf1 as referred to herein has a sequence homology or identity of at least 80%, more preferably at least 85%, even more preferably at least 90%, such as for instance at least 95% with Cpf1. In further embodiments, the homologue or orthologue of Cpf1 as referred to herein has a sequence identity of at least 80%, more preferably at least 85%, even more preferably at least 90%, such as for instance at least 95% with the wild type Cpf1. Where the Cpf1 has one or more mutations (mutated), the homologue or orthologue of said Cpf1 as referred to herein has a sequence identity of at least 80%, more preferably at least 85%, even more preferably at least 90%, such as for instance at least 95% with the mutated Cpf1.

In an embodiment, the Cpf1 protein may be an ortholog of an organism of a genus which includes, but is not limited to Acidaminococcus sp, Lachnospiraceae bacterium or Moraxella bovoculi; in particular embodiments, the type V Cas protein may be an ortholog of an organism of a species which includes, but is not limited to Acidaminococcus sp. BV3L6; Lachnospiraceae bacterium ND2006 (LbCpf1) or Moraxella bovoculi 237.In particular embodiments, the homologue or orthologue of Cpf1 as referred to herein has a sequence homology or identity of at least 80%, more preferably at least 85%, even more preferably at least 90%, such as for instance at least 95% with one or more of the Cpf1 sequences disclosed herein. In further embodiments, the homologue or orthologue of Cpf as referred to herein has a sequence identity of at least 80%, more preferably at least 85%, even more preferably at least 90%, such as for instance at least 95% with the wild type FnCpf1, AsCpf1 or LbCpf1. The skilled person will understand that this includes truncated forms of the Cpf1 protein whereby the sequence identity is determined over the length of the truncated form. In certain of the following, Cpf1 amino acids are followed by nuclear localization signals (NLS) (italics), a glycine-serine (GS) linker, and 3× HA tag. Further Cpf1 orthologs include NCBI WP_055225123.1, NCBI WP_055237260.1, NCBI WP_055272206.1, and GenBank OLA16049.1.

Cas 12b Orthologs

The present invention encompasses the use of a Cas 12b (C2c1) effector proteins, derived from a C2c1 locus denoted as subtype V-B. Herein such effector proteins are also referred to as “C2c1p”, e.g., a C2c1 protein (and such effector protein or C2c1 protein or protein derived from a C2c1 locus is also called “CRISPR enzyme”). Presently, the subtype V-B loci encompasses cas1-Cas4 fusion, cas2, a distinct gene denoted C2c1 and a CRISPR array. C2c1 (CRISPR-associated protein C2c1) is a large protein (about 1100-1300 amino acids) that contains a RuvC-like nuclease domain homologous to the corresponding domain of Cas9 along with a counterpart to the characteristic arginine-rich cluster of Cas9. However, C2c1 lacks the HNH nuclease domain that is present in all Cas9 proteins, and the RuvC-like domain is contiguous in the C2c1 sequence, in contrast to Cas9 where it contains long inserts including the HNH domain. Accordingly, in particular embodiments, the CRISPR-Cas enzyme comprises only a RuvC-like nuclease domain.

The programmability, specificity, and collateral activity of the RNA-guided C2c1 also make it an ideal switchable nuclease for non-specific cleavage of nucleic acids. In one embodiment, a C2c1 system is engineered to provide and take advantage of collateral non-specific cleavage of RNA. In another embodiment, a C2c1 system is engineered to provide and take advantage of collateral non-specific cleavage of ssDNA. Accordingly, engineered C2c1 systems provide platforms for nucleic acid detection and transcriptome manipulation, and inducing cell death. C2c1 is developed for use as a mammalian transcript knockdown and binding tool. C2c1 is capable of robust collateral cleavage of RNA and ssDNA when activated by sequence-specific targeted DNA binding.

In certain embodiments, C2c1 is provided or expressed in an in vitro system or in a cell, transiently or stably, and targeted or triggered to non-specifically cleave cellular nucleic acids. In one embodiment, C2c1 is engineered to knock down ssDNA, for example viral ssDNA. In another embodiment, C2c1 is engineered to knock down RNA. The system can be devised such that the knockdown is dependent on a target DNA present in the cell or in vitro system, or triggered by the addition of a target nucleic acid to the system or cell.

C2c1 (also known as Cas12b) proteins are RNA guided nucleases. In certain embodiments, the Cas protein may comprise at least 80% sequence identity to a polypeptide as described in International Patent Publication WO 2016/205749 at FIG. 17-21, FIG. 41A-41M, 44A-44E, incorporated herein by reference. Its cleavage relies on a tracr RNA to recruit a guide RNA comprising a guide sequence and a direct repeat, where the guide sequence hybridizes with the target nucleotide sequence to form a DNA/RNA heteroduplex. Based on current studies, C2c1 nuclease activity also requires relies on recognition of PAM sequence. C2c1 PAM sequences are T-rich sequences. In some embodiments, the PAM sequence is 5′ TTN 3′ or 5′ ATTN 3′, wherein N is any nucleotide. In a particular embodiment, the PAM sequence is 5′ TTC 3′. In a particular embodiment, the PAM is in the sequence of Plasmodium falciparum.

In particular embodiments, the effector protein is a C2c1 effector protein from an organism from a genus comprising Alicyclobacillus, Desulfovibrio, Desulfonatronum, Opitutaceae, Tuberibacillus, Bacillus, Brevibacillus, Candidatus, Desulfatirhabdium, Citrobacter, Elusimicrobia, Methylobacterium, Omnitrophica, Phycisphaerae, Planctomycetes, Spirochaetes, and Verrucomicrobiaceae.

In further particular embodiments, the C2c1 effector protein is from a species selected from Alicyclobacillus acidoterrestris (e.g., ATCC 49025), Alicyclobacillus contaminans (e.g., DSM 17975), Alicyclobacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAVS, Phycisphaerae bacterium ST-NAGAB-D1, Planctomycetes bacterium RBG 13 46 10, Spirochaetes bacterium GWB1_27_13, Verrucomicrobiaceae bacterium UBA2429, Tuberibacillus calidus (e.g., DSM 17572), Bacillus thermoamylovorans (e.g., strain B4166), Brevibacillus sp. CF112, Bacillus sp. NSP2.1, Desulfatirhabdium butyrativorans (e.g., DSM 18734), Alicyclobacillus herbarius (e.g., DSM 13609), Citrobacter freundii (e.g., ATCC 8090), Brevibacillus agri (e.g., BAB-2500), Methylobacterium nodulans (e.g., ORS 2060).

The effector protein may comprise a chimeric effector protein comprising a first fragment from a first effector protein (e.g., a C2c1) ortholog and a second fragment from a second effector (e.g., a C2c1) protein ortholog, and wherein the first and second effector protein orthologs are different. At least one of the first and second effector protein (e.g., a C2c1) orthologs may comprise an effector protein (e.g., a C2c1) from an organism comprising Alicyclobacillus, Desulfovibrio, Desulfonatronum, Opitutaceae, Tuberibacillus, Bacillus, Brevibacillus, Candidatus, Desulfatirhabdium, Elusimicrobia, Citrobacter, Methylobacterium, Omnitrophicai, Phycisphaerae, Planctomycetes, Spirochaetes, and Verrucomicrobiaceae; e.g., a chimeric effector protein comprising a first fragment and a second fragment wherein each of the first and second fragments is selected from a C2c1 of an organism comprising Alicyclobacillus, Desulfovibrio, Desulfonatronum, Opitutaceae, Tuberibacillus, Bacillus, Brevibacillus, Candidatus, Desulfatirhabdium, Elusimicrobia, Citrobacter, Methylobacterium, Omnitrophicai, Phycisphaerae, Planctomycetes, Spirochaetes, and Verrucomicrobiaceae wherein the first and second fragments are not from the same bacteria; for instance a chimeric effector protein comprising a first fragment and a second fragment wherein each of the first and second fragments is selected from a C2c1 of Alicyclobacillus acidoterrestris (e.g., ATCC 49025), Alicyclobacillus contaminans (e.g., DSM 17975), Alicyclobacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAV5, Phycisphaerae bacterium ST-NAGAB-D1, Planctomycetes bacterium RBG_13-46_10, Spirochaetes bacterium GWB1_27_13, Verrucomicrobiaceae bacterium UBA2429, Tuberibacillus calidus (e.g., DSM 17572), Bacillus thermoamylovorans (e.g., strain B4166), Brevibacillus sp. CF112, Bacillus sp. NSP2.1, Desulfatirhabdium butyrativorans (e.g., DSM 18734), Alicyclobacillus herbarius (e.g., DSM 13609), Citrobacter freundii (e.g., ATCC 8090), Brevibacillus agri (e.g., BAB-2500), Methylobacterium nodulans (e.g., ORS 2060), wherein the first and second fragments are not from the same bacteria.

In a more preferred embodiment, the C2c1p is derived from a bacterial species selected from Alicyclobacillus acidoterrestris (e.g., ATCC 49025), Alicyclobacillus contaminans (e.g., DSM 17975), Alicyclobacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAV5, Phycisphaerae bacterium ST-NAGAB-D1, Planctomycetes bacterium RBG_13_46_10, Spirochaetes bacterium GWB1_27_13, Verrucomicrobiaceae bacterium UBA2429, Tuberibacillus calidus (e.g., DSM 17572), Bacillus thermoamylovorans (e.g., strain B4166), Brevibacillus sp. CF112, Bacillus sp. NSP2.1, Desulfatirhabdium butyrativorans (e.g., DSM 18734), Alicyclobacillus herbarius (e.g., DSM 13609), Citrobacter freundii (e.g., ATCC 8090), Brevibacillus agri (e.g., BAB-2500), Methylobacterium nodulans (e.g., ORS 2060). In certain embodiments, the C2c1p is derived from a bacterial species selected from Alicyclobacillus acidoterrestris (e.g., ATCC 49025), Alicyclobacillus contaminans (e.g., DSM 17975).

In particular embodiments, the homologue or orthologue of C2c1 as referred to herein has a sequence homology or identity of at least 80%, more preferably at least 85%, even more preferably at least 90%, such as for instance at least 95% with C2c1. In further embodiments, the homologue or orthologue of C2c1 as referred to herein has a sequence identity of at least 80%, more preferably at least 85%, even more preferably at least 90%, such as for instance at least 95% with the wild type C2c1. Where the C2c1 has one or more mutations (mutated), the homologue or orthologue of said C2c1 as referred to herein has a sequence identity of at least 80%, more preferably at least 85%, even more preferably at least 90%, such as for instance at least 95% with the mutated C2c1.

In an embodiment, the C2c1 protein may be an ortholog of an organism of a genus which includes, but is not limited to Alicyclobacillus, Desulfovibrio, Desulfonatronum, Opitutaceae, Tuberibacillus, Bacillus, Brevibacillus, Candidatus, Desulfatirhabdium, Elusimicrobia, Citrobacter, Methylobacterium, Omnitrophicai, Phycisphaerae, Planctomycetes, Spirochaetes, and Verrucomicrobiaceae; in particular embodiments, the type V Cas protein may be an ortholog of an organism of a species which includes, but is not limited to Alicyclobacillus acidoterrestris (e.g., ATCC 49025), Alicyclobacillus contaminans (e.g., DSM 17975), Alicyclobacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAVS, Phycisphaerae bacterium ST-NAGAB-D 1, Planctomycetes bacterium RBG_13_46_10, Spirochaetes bacterium GWB1_27_13, Verrucomicrobiaceae bacterium UBA2429, Tuberibacillus calidus (e.g., DSM 17572), Bacillus thermoamylovorans (e.g., strain B4166), Brevibacillus sp. CF112, Bacillus sp. NSP2.1, Desulfatirhabdium butyrativorans (e.g., DSM 18734), Alicyclobacillus herbarius (e.g., DSM 13609), Citrobacter freundii (e.g., ATCC 8090), Brevibacillus agri (e.g., BAB-2500), Methylobacterium nodulans (e.g., ORS 2060). In particular embodiments, the homologue or orthologue of C2c1 as referred to herein has a sequence homology or identity of at least 80%, more preferably at least 85%, even more preferably at least 90%, such as for instance at least 95% with one or more of the C2c1 sequences disclosed herein. In further embodiments, the homologue or orthologue of C2c1 as referred to herein has a sequence identity of at least 80%, more preferably at least 85%, even more preferably at least 90%, such as for instance at least 95% with the wild type AacC2c1 or BthC2c1.

In particular embodiments, the C2c1 protein of the invention has a sequence homology or identity of at least 60%, more particularly at least 70, such as at least 80%, more preferably at least 85%, even more preferably at least 90%, such as for instance at least 95% with AacC2c1 or BthC2c1. In further embodiments, the C2c1 protein as referred to herein has a sequence identity of at least 60%, such as at least 70%, more particularly at least 80%, more preferably at least 85%, even more preferably at least 90%, such as for instance at least 95% with the wild type AacC2c1. In particular embodiments, the C2c1 protein of the present invention has less than 60% sequence identity with AacC2c1. The skilled person will understand that this includes truncated forms of the C2c1 protein whereby the sequence identity is determined over the length of the truncated form.

In certain methods according to the present invention, the CRISPR-Cas protein is preferably mutated with respect to a corresponding wild-type enzyme such that the mutated CRISPR-Cas protein lacks the ability to cleave one or both DNA strands of a target locus containing a target sequence. In particular embodiments, one or more catalytic domains of the C2c1 protein are mutated to produce a mutated Cas protein which cleaves only one DNA strand of a target sequence.

In particular embodiments, the CRISPR-Cas protein may be mutated with respect to a corresponding wild-type enzyme such that the mutated CRISPR-Cas protein lacks substantially all DNA cleavage activity. In some embodiments, a CRISPR-Cas protein may be considered to substantially lack all DNA and/or RNA cleavage activity when the cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form.

In certain embodiments of the methods provided herein the CRISPR-Cas protein is a mutated CRISPR-Cas protein which cleaves only one DNA strand, i.e. a nickase. More particularly, in the context of the present invention, the nickase ensures cleavage within the non-target sequence, i.e. the sequence which is on the opposite DNA strand of the target sequence and which is 3′ of the PAM sequence. By means of further guidance, and without limitation, an arginine-to-alanine substitution (R911A) in the Nuc domain of C2c1 from Alicyclobacillus acidoterrestris converts C2c1 from a nuclease that cleaves both strands to a nickase (cleaves a single strand). It will be understood by the skilled person that where the enzyme is not AacC2c1, a mutation may be made at a residue in a corresponding position.

### Cas 12c Orthologs

In certain embodiments, the effector protein, particularly a Type V loci effector protein, more particularly a Type V-C loci effector protein, a Cas12c protein, even more particularly a C2c3p, may originate, may be isolated or may be derived from a bacterial metagenome selected from the group consisting of the bacterial metagenomes listed in the Table in FIG. 43A-43B of PCT/US2016/038238, specifically incorporated by reference, which presents analysis of the Type-V-C Cas12c loci.

In certain embodiments, the effector protein, particularly a Type V loci effector protein, more particularly a Type V-C loci effector protein, even more particularly a C2c3p, may comprise, consist essentially of or consist of an amino acid sequence selected from the group consisting of amino acid sequences shown in the multiple sequence alignment in FIG. 131 of PCT/US2016/038238, specifically incorporated by reference.

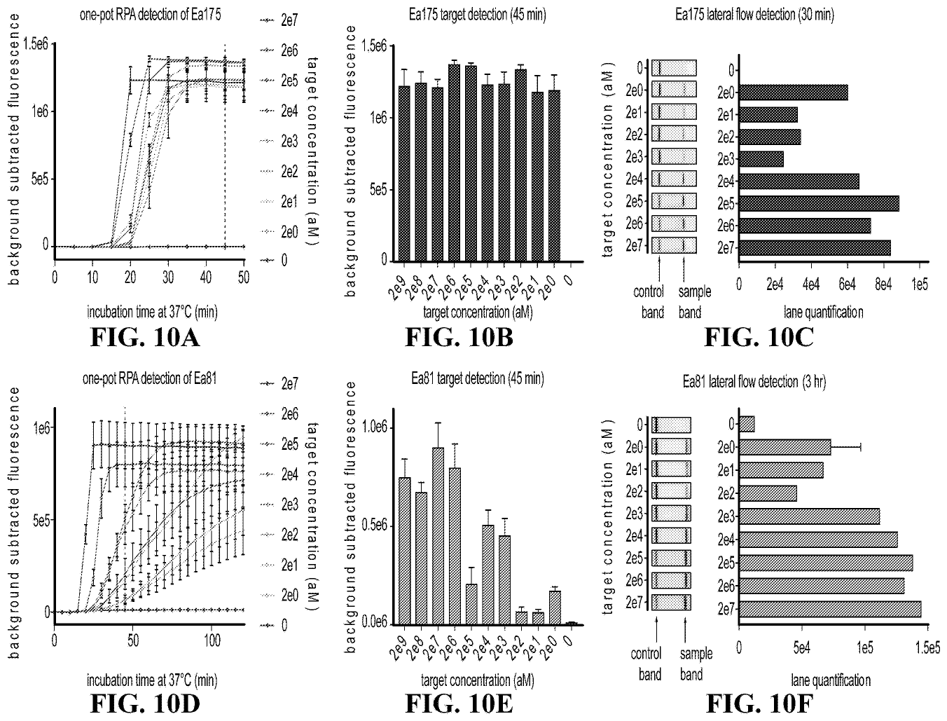

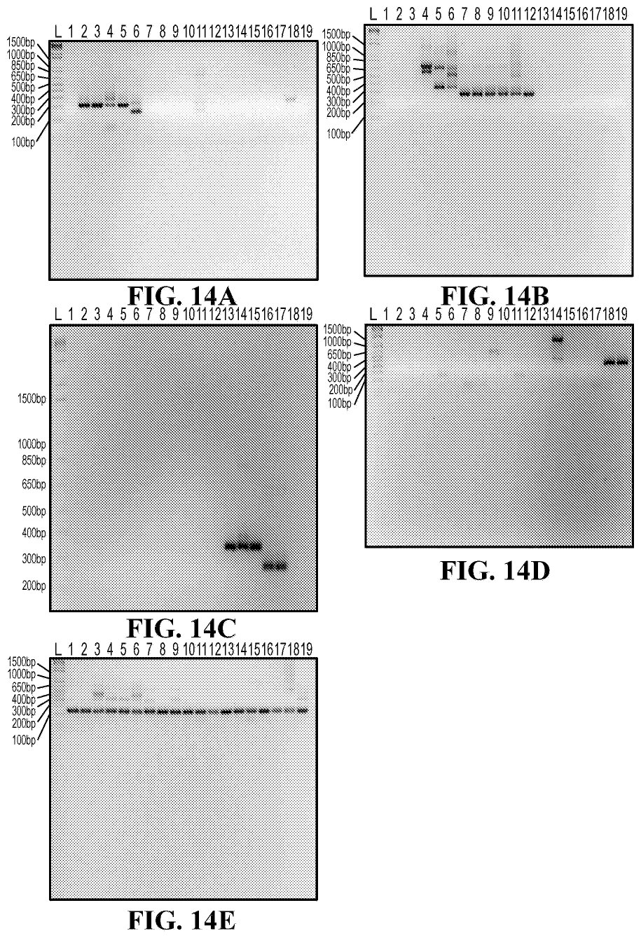

In certain embodiments, a Type V-C locus as intended herein may encode Cas1 and the C2c3p effector protein. See FIG. 14 of PCT/US2016/038238, specifically incorporated by reference, depicting the genomic architecture of the Cas12c CRISPR-Cas loci. In certain embodiments, a Cas1 protein encoded by a Type V-C locus as intended herein may cluster with Type I-B system. See FIG. 10A and 10B and FIG. 10C-V of PCT/US2016/038238, specifically incorporated by reference, illustrating a Cas1 tree including Cas1 encoded by representative Type V-C loci.

In certain embodiments, the effector protein, particularly a Type V loci effector protein, more particularly a Type V-C loci effector protein, even more particularly a C2c3p, such as a native C2c3p, may be about 1100 to about 1500 amino acids long, e.g., about 1100 to about 1200 amino acids long, or about 1200 to about 1300 amino acids long, or about 1300 to about 1400 amino acids long, or about 1400 to about 1500 amino acids long, e.g., about 1100, about 1200, about 1300, about 1400 or about 1500 amino acids long, or at least about 1100, at least about 1200, at least about 1300, at least about 1400 or at least about 1500 amino acids long.

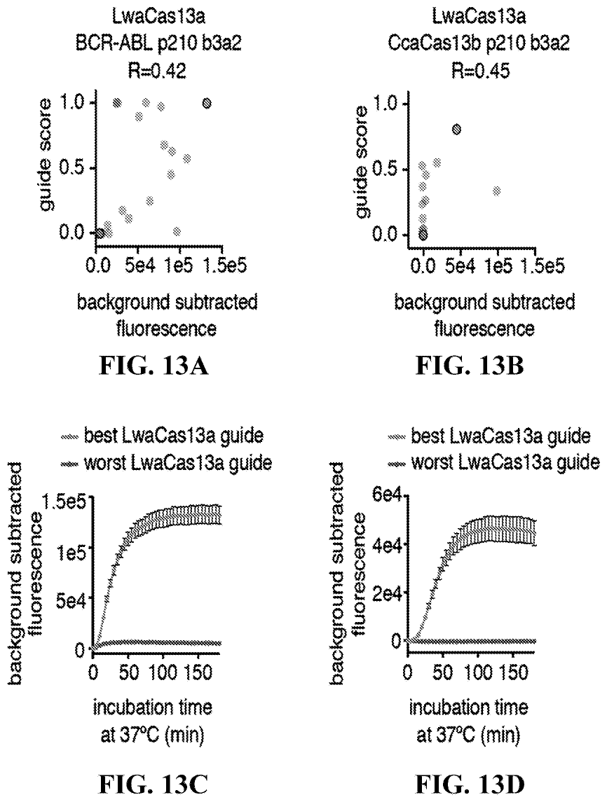

In certain embodiments, the effector protein, particularly a Type V loci effector protein, more particularly a Type V-C loci effector protein, even more particularly a C2c3p, and preferably the C-terminal portion of said effector protein, comprises the three catalytic motifs of the RuvC-like nuclease (i.e., RuvCI, RuvCII and RuvCIII). In certain embodiments, said effector protein, and preferably the C-terminal portion of said effector protein, may further comprise a region corresponding to the bridge helix (also known as arginine-rich cluster) that in Cas9 protein is involved in crRNA-binding. In certain embodiments, said effector protein, and preferably the C-terminal portion of said effector protein, may further comprise a Zn finger region. Preferably, the Zn-binding cysteine residue(s) may be conserved in C2c3p. In certain embodiments, said effector protein, and preferably the C-terminal portion of said effector protein, may comprise the three catalytic motifs of the RuvC-like nuclease (i.e., RuvCI, RuvCII and RuvCIII), the region corresponding to the bridge helix, and the Zn finger region, preferably in the following order, from N to C terminus: RuvCI-bridge helix-RuvCII-Zinc finger-RuvCIII. See FIG. 13A and 13C of PCT/US2016/038238, specifically incorporated by reference, for illustration of representative Type V-C effector proteins domain architecture.

In certain embodiments, Type V-C loci as intended herein may comprise CRISPR repeats between 20 and 30 bp long, more typically between 22 and 27 bp long, yet more typically 25 bp long, e.g., 20, 21, 22, 23, 24, 25, 26, 27, 28, 29, or 30 bp long.

Orthologous proteins may but need not be structurally related, or are only partially structurally related. In particular embodiments, the homologue or orthologue of a Type V protein such as Cas12c as referred to herein has a sequence homology or identity of at least 80%, more preferably at least 85%, even more preferably at least 90%, such as for instance at least 95% with a Cas12c. In further embodiments, the homologue or orthologue of a Type V Cas12c as referred to herein has a sequence identity of at least 80%, more preferably at least 85%, even more preferably at least 90%, such as for instance at least 95% with the wild type Cas12c.

In an embodiment, the Type V RNA-targeting Cas protein may be a Cas12c ortholog of an organism of a genus which includes but is not limited to Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter.

In an embodiment, the Cas12c or an ortholog or homolog thereof, may comprise one or more mutations (and hence nucleic acid molecule(s) coding for same may have mutation(s). The mutations may be artificially introduced mutations and may include but are not limited to one or more mutations in a catalytic domain. Examples of catalytic domains with reference to a Cas enzyme may include but are not limited to RuvC I, RuvC II, RuvC III, HNH domains, and HEPN domains, as described herein. In an embodiment, the Cas12c or an ortholog or homolog thereof, may comprise one or more mutations. The mutations may be artificially introduced mutations and may include but are not limited to one or more mutations in a catalytic domain.

### Guide Sequences

As used herein, the term “guide sequence” and “guide molecule” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that a RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), Clustal W, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

As used herein, the term “guide sequence,” “crRNA,” “guide RNA,” or “single guide RNA,” or “gRNA” refers to a polynucleotide comprising any polynucleotide sequence having sufficient complementarity with a target nucleic acid sequence to hybridize with the target nucleic acid sequence and to direct sequence-specific binding of a RNA-targeting complex comprising the guide sequence and a CRISPR effector protein to the target nucleic acid sequence. In some example embodiments, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), Clustal W, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible, and will occur to those skilled in the art. A guide sequence, and hence a nucleic acid-targeting guide may be selected to target any target nucleic acid sequence. The target sequence may be DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within a RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In some embodiments, it is of interest to reduce the susceptibility of the guide molecule to RNA cleavage, such as to cleavage by Cas13. Accordingly, in particular embodiments, the guide molecule is adjusted to avoid cleavage by Cas13 or other RNA-cleaving enzymes.

In certain embodiments, the guide molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA). Other examples of modified nucleotides include 2′-0-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′phosphorothioate (MS), S-constrained ethyl(cEt), or 2′-O-methyl 3′thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target RNA and one or more deoxyribonucleotides and/or nucleotide analogs in a region that binds to Cas13. In an embodiment of the invention, deoxyribonucleotides and/or nucleotide analogs are incorporated in engineered guide structures, such as, without limitation, stem-loop regions, and the seed region. For Cas13 guide, in certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554).

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A.R. Gruber et al., 2008, Cell 106(1): 23-24; and P A Carr and G M Church, 2009, Nature Biotechnology 27(12): 1151-62).

In some embodiments, a nucleic acid-targeting guide is designed or selected to modulate intermolecular interactions among guide molecules, such as among stem-loop regions of different guide molecules. It will be appreciated that nucleotides within a guide that base-pair to form a stem-loop are also capable of base-pairing to form an intermolecular duplex with a second guide and that such an intermolecular duplex would not have a secondary structure compatible with CRISPR complex formation. Accordingly, it is useful to select or design DR sequences in order to modulate stem-loop formation and CRISPR complex formation. In some embodiments, about or less than about 75%, 50%, 40%, 30%, 25%, 20%, 15%, 10%, 5%, 1%, or fewer of nucleic acid-targeting guides are in intermolecular duplexes. It will be appreciated that stem-loop variation will often be within limits imposed by DR-CRISPR effector interactions. One way to modulate stem-loop formation or change the equilibrium between stem-loop and intermolecular duplex is to vary nucleotide pairs in the stem of the stem-loop of a DR. For example, in one embodiment, a G-C pair is replaced by an A-U or U-A pair. In another embodiment, an A-U pair is substituted for a G-C or a C-G pair. In another embodiment, a naturally occurring nucleotide is replaced by a nucleotide analog. Another way to modulate stem-loop formation or change the equilibrium between stem-loop and intermolecular duplex is to modify the loop of the stem-loop of a DR. Without be bound by theory, the loop can be viewed as an intervening sequence flanked by two sequences that are complementary to each other. When that intervening sequence is not self-complementary, its effect will be to destabilize intermolecular duplex formation. The same principle applies when guides are multiplexed: while the targeting sequences may differ, it may be advantageous to modify the stem-loop region in the DRs of the different guides. Moreover, when guides are multiplexed, the relative activities of the different guides can be modulated by balancing the activity of each individual guide. In certain embodiments, the equilibrium between intermolecular stem-loops vs. intermolecular duplexes is determined. The determination may be made by physical or biochemical means and can be in the presence or absence of a CRISPR effector.

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer.

In general, the CRISPR-Cas, CRISPR-Cas9 or CRISPR system may be as used in the foregoing documents, such as International Patent Publication No. WO 2014/093622 (PCT/US2013/074667) and refers collectively to transcripts and other elements involved in the expression of or directing the activity of CRISPR-associated (“Cas”) genes, including sequences encoding a Cas gene, in particular a Cas9 gene in the case of CRISPR-Cas9, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. The section of the guide sequence through which complementarity to the target sequence is important for cleavage activity is referred to herein as the seed sequence. A target sequence may comprise any polynucleotide, such as DNA or RNA polynucleotides. In some embodiments, a target sequence is located in the nucleus or cytoplasm of a cell, and may include nucleic acids in or from mitochondrial, organelles, vesicles, liposomes or particles present within the cell. In some embodiments, especially for non-nuclear uses, NLSs are not preferred. In some embodiments, a CRISPR system comprises one or more nuclear exports signals (NESs). In some embodiments, a CRISPR system comprises one or more NLSs and one or more NESs. In some embodiments, direct repeats may be identified in silico by searching for repetitive motifs that fulfill any or all of the following criteria: 1. found in a 2Kb window of genomic sequence flanking the type II CRISPR locus; 2. span from 20 to 50 bp; and 3. interspaced by 20 to 50 bp. In some embodiments, 2 of these criteria may be used, for instance 1 and 2, 2 and 3, or 1 and 3. In some embodiments, all 3 criteria may be used.

In embodiments of the invention the terms guide sequence and guide RNA, i.e. RNA capable of guiding Cas to a target genomic locus, are used interchangeably as in foregoing cited documents such as International Patent Publication WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably, the guide sequence is 10 30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In some embodiments of CRISPR-Cas systems, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and advantageously tracr RNA is 30 or 50 nucleotides in length. However, an aspect of the invention is to reduce off-target interactions, e.g., reduce the guide interacting with a target sequence having low complementarity. Indeed, in the examples, it is shown that the invention involves mutations that result in the CRISPR-Cas system being able to distinguish between target and off-target sequences that have greater than 80% to about 95% complementarity, e.g., 83%-84% or 88-89% or 94-95% complementarity (for instance, distinguishing between a target having 18 nucleotides from an off-target of 18 nucleotides having 1, 2 or 3 mismatches). Accordingly, in the context of the present invention the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

### Cancer Fusion Genes

Fusion genes and their transcripts are chimeras resulting from separate genes with aberrant functions, with the results transcript leading to potential aberrant expression levels, functions and sites, many of which have been identified in various cancer types. Due to the oncogenic potential of the chimeric protein generated through fusions. Sources of cancer fusion genes and research describing can be found, for example, in the Catalogue of Somatic Mutations in Cancer (SOMATIC), available at cancer.sanger.ac.uk/cosmic/fusion.

Exemplary cancer fusions that can be used in accordance with the present invention include: ZSCAN30_ENST00000333206-BRAF, ZNF700_ENST00000254321-MAST1, ZCCHC8-ROS1, ZC3H7B-BCOR, YWHAE-NUTM2B, YWHAE-NUTM2A, WDCP-ALK, VTI1A-TCF7L2_ENST00000369397, VCL-ALK, UBE2L3_ENST00000342192-KRAS_ENST00000311936, TRIM33-RET, TRIM27-RET, TRIM24-RET, TRIM24-BRAF, TPR-NTRK1_ENST00000392302, TPR-ALK, TPM4_ENST00000300933-ALK, TPM3_ENST00000368533-ROS1, TPM3_ENST00000368533-NTRK1_ENST00000392302, TPM3_ENST00000368533-ALK, TPM3-ROS1, TP53-NTRK1_ENST00000392302, TMPRSS2_ENST00000332149-ETV5, TMPRSS2_ENST00000332149-ETV4, TMPRSS2_ENST00000332149-ETV1, TMPRSS2_ENST00000332149-ERG_ENST00000442448, THRAP3-USP6_ENST00000250066, TFG-NTRK1_ENST00000392302, TFG-NR4A3 ENST00000395097, TFG-ALK, TECTA-TBCEL_ENST00000529397, TCF3-PBX1, TCF12-NR4A3_ENST00000395097, TCEA1-PLAG1, TBL1XR1-TP63, TAF15_ENST00000604841-NR4A3_ENST00000395097, TADA2A-MAST1, SUSD1_ENST00000374270-PTBP3_ENST00000374255, STRN-ALK, STIL_ENST00000360380-TAL1_ENST00000371884, SSH2_ENST00000269033-SUZ12, SSBP2_ENST00000320672-JAK2, SS18L1-SSX1, SS18-USP6_ENST00000250066, SS18-SSX4B, SS18-SSX2, SS18-SSX1, SRGAP3-RAF1, SQSTM1-ALK, SND1-BRAF, SLC45A3-ETV5, SLC45A3-ETV1, SLC45A3-ERG_ENST00000442448, SLC45A3-ELK4, SLC45A3-BRAF, SLC3A2-NRG1, SLC34A2-ROS1, SLC26A6-PRKAR2A, SLC22A1-CUTA_ENST00000440279, SHTN1_ENST00000615301-ROS1, SFPQ-TFE3, SET_ENST00000322030-NUP214, SEPT8_ENST00000296873-AFF4, SEC31A_ENST00000348405-JAK2, SEC31A_ENST00000348405-ALK, SEC16A-NOTCH1, SDC4-ROS1, RUNX1-RUNX1T1_ENST00000360348, RNF130-BRAF, RGS22-SYCP1_ENST00000369518, RELCH-RET,

RBM14-PACS1, RANBP2-ALK, RAF1-DAZL_ENST00000399444, QKI-NTRK2_ENST00000376214, PWWP2A_ENST00000456329-ROS1, PTPRK_ENST00000368226-RSPO3, PRKAR1A_ENST00000358598-RET, PRCC-TFE3, PPFIBP1_ENST00000228425-ROS1, PPFIBP1_ENST00000228425-ALK, PML-RARA, PLXND1-TMCC1, PLA2R1-RBMS1, PCM1-RET, PCM1-JAK2, PAX8_ENST00000429538-PPARG, PAX7-FOXO1, PAX5-JAK2, PAX3-NCOA2, PAX3-NCOA1, PAX3-FOXO1, OMD-USP6_ENST00000250066, NUP98-KDM5A, NUP214-ABL1_ENST00000318560, NUP107-LGR5, NTN1-ACLY, NPM1_ENST00000517671-ALK, NOTCH1-GABBR2, NONO-TFE3, NFIX_ENST00000360105-MAST1, NFIA_ENST00000485903-EHF_ENST00000257831, NF1-ASIC2, NDRG1_ENST00000323851-ERG_ENST00000442448, NCOA4-RET, NACC2_ENST00000371753-NTRK2_ENST00000376214, NAB2-STAT6, MYO5A-ROS1, MYB-NFIB_ENST00000397581, MSN-ALK, MN1-ETV6, MKRN1-BRAF, MIA2_ENST00000280083-GEMIN2, MEAF6-PHF1, MBTD1-CXorf67, MBOAT2-PRKCE, LSM14A-BRAF, LRIG3 -ROS1, LMNA-NTRK1_ENST00000392302, LIFR-PLAG1, KTN1-RET, KMT2A-ZFYVE19, KMT2A-TOP3A ENST00000321105, KMT2A-TET1, KMT2A-SORB S2_ENST00000284776, KMT2A-SH3GL1, KMT2A-SEPT9, KMT2A-SEPT6, KMT2A-SEPTS, KMT2A-SEPT2_ENST00000360051, KMT2A-SARNP, KMT2A-PRRC1_ENST00000296666, KMT2A-PICALM, KMT2A-PD S 5A, KMT2A-NRIP3, KMT2A-NCKIPSD, KMT2A-MYO1F, KMT2A-MLLT6, KMT2A-MLLT3, KMT2A-MLLT11, KMT2A-MLLT10_ENST00000377072, KMT2A-MLLT1, KMT2A-MAPRE1, KMT2A-LPP, KMT2A-LASP1, KMT2A-KNL1, KMT2A-GPHN, KMT2A-GMPS, KMT2A-GAS7, KMT2A-FRYL, KMT2A-FOXO4, KMT2A-FOXO3 ENST00000343882, KMT2A-EPS15, KMT2A-EP300, KMT2A-ELL, KMT2A-EEFSEC, KMT2A-DAB2IP_ENST00000309989, KMT2A-CT45A2_ENST00000612907, KMT2A-CREBBP, KMT2A-CIP2A, KMT2A-CEP170B, KMT2A-CBL, KMT2A-CASP8AP2, KMT2A-BTBD18, KMT2A-ARHGEF12, KMT2A-ARHGAP26, KMT2A-AFF4, KMT2A-AFF3, KMT2A-AFF1_ENST00000307808, KMT2A-AFDN_ENST00000392108, KMT2A-ACTN4, KMT2A-ABI2_ENST00000261017, KMT2A-ABI1, KLK2-ETV4, KLK2-ETV1, KLC1_ENST00000389744-ALK, KIFSB-RET, KIFSB-ALK, KIAA1549_ENST00000440172-BRAF, JPT1-USH1G, JAZF 1-SUZ12, JAZF 1-PHF 1, IRF2BP2-CDX1, INTS4-GAB2, IL6R-ATP8B2, HOOK3-RET, HNRNPA2B1_ENST00000356674-ETV1, HMGA2_ENST00000403681-WIF 1, HMGA2_ENST00000403681-RAD51B_ENST00000487270, HMGA2_ENST00000403681-NFIB_ENST00000397581, HMGA2_ENST00000403681-LPP, HMGA2_ENST00000403681-LHFPL6, HMGA2_ENST00000403681-FHIT_ENST00000476844, HMGA2_ENST00000403681-EBF 1, HMGA2_ENST00000403681-COX6C_ENST00000297564, HMGA2_ENST00000403681-CCNB1IP1_ENST00000358932, HMGA2_ENST00000403681-ALDH2, HLA-A-ROS1, HIP1-ALK, HEY1_ENST00000354724-NCOA2, HERPUD1_ENST00000300302-BRAF, HAS2-PLAG1, HACL1-RAF1, GPBP1L1_ENST00000290795-MAST2, GOPC-ROS1, GOLGAS-RET, GNAI1-BRAF, GMDS-PDE8B, GATM-BRAF, FUS-FEV, FUS-ERG_ENST00000442448, FUS-DDIT3 ENST00000547303, FUS-CREB3L2, FUS-CREB3L1, FUS-ATF1, FN1 ENST00000336916-ALK, FGFR3_ENST00000440486-TACC3, FGFR3_ENST00000440486-BAIAP2L1, FGER1_ENST00000447712-ZNF703, FGFR1_ENST00000447712-TACC1, FGFR1_ENST00000447712-PLAG1, FCHSD1-BRAF, FBXL18-RNF216, FAM131B-BRAF, EZR-ROS1, EZR-ERBB4, EWSR1_ENST00000397938-ZNF444, EWSR1_ENST00000397938-ZNF384_ENST00000319770, EWSR1_ENST00000397938-YY1, EWSR1_ENST00000397938-WT1, EWSR1_ENST00000397938-SP3, EWSR1_ENST00000397938-SMARCA5, EWSR1_ENST00000397938-POU5F1, EWSR1_ENST00000397938-PBX1, EWSR1_ENST00000397938-PATZ1_ENST00000215919, EWSR1_ENST00000397938-NR4A3_ENST00000395097, EWSR1_ENST00000397938-NFATC2, EWSR1_ENST00000397938-NFATC1_ENST00000329101, EWSR1_ENST00000397938-MYB, EWSR1_ENST00000397938-FLI1, EWSR1_ENST00000397938-FEV, EWSR1_ENST00000397938-ETV4, EWSR1_ENST00000397938-ETV1, EWSR1_ENST00000397938-ERG ENST00000442448, EWSR1_ENST00000397938-DDIT3_ENST00000547303, EWSR1_ENST00000397938-CREB1, EWSR1_ENST00000397938-ATF 1, ETV6-RUNX1, ETV6-PDGFRB, ETV6-NTRK3_ENST00000394480, ETV6-JAK2, ETV6-ITPR2, ETV6-ABL1_ENST00000318560, ESRP1_ENST00000358397-RAF1, ERO1A-FERMT2_ENST00000395631, ERC1_ENST00000360905-ROS1, ERC1_ENST00000360905-RET, EPC1-PHF1, EML4-ALK, EIF3K-CYP39A1, EIF3E-RSPO2, DNAJB1-PRKACA, DHH-RHEBL1, DDXS-ETV4, DCTN1-ALK, CTNNB1 ENST00000349496-PLAG1, CRTC3-MAML2, CRTC1 ENST00000321949-MAML2, COL1A2-PLAG1, COL1A1 -USP6_ENST00000250066, COL1A1-PDGFB, CNBP_ENST00000422453-USP6_ENST00000250066, CLTC_ENST00000269122-TFE3, CLTC ENST00000269122-ALK, CLIP1_ENST00000358808-ROS1, CLCN6-BRAF, CIC_ENST00000160740-FOXO4, CIC_ENST00000160740-DUX4, CHCHD7_ENST00000355315-PLAG1, CEP89-BRAF, CENPK-KMT2A, CDKN2D ENST00000335766-WDFY2, CDH11-USP6 ENST00000250066, CD74-ROS1, CD74-NRG1, CCDC6-RET, CBFA2T3-GLIS2, CARS ENST00000397111-ALK, CANT1_ENST00000392446-ETV4, BRD4-NUTM1_ENST00000333756, BRD3-NUTM1_ENST00000333756, BCR-JAK2, BCR-ABL1_ENST00000318560, BBS9-PKD1L1, ATIC-ALK, ATG4C ENST00000371120-FBXO38_ENST00000340253, ASPSCR1_ENST00000306739-TFE3, ARID1A-MAST2, ARFIP1_ENST00000353617-FHDC1_ENST00000260008, AKAP9-BRAF, AGTRAP-BRAF, AGPATS-MCPH1, ACTB-GLI1, ACSL3-ETV1, and ACBD6 ENST00000367595-RRP15. Target sequences of the fusion genes can be identified and utilized for generating optimized guide according to the presently disclosed methods. The optimized guides can optionally be used with amplification reagents that may provide further specifically designed primers for the target sequence, fusion gene, or specific translocation for the identified cancer.

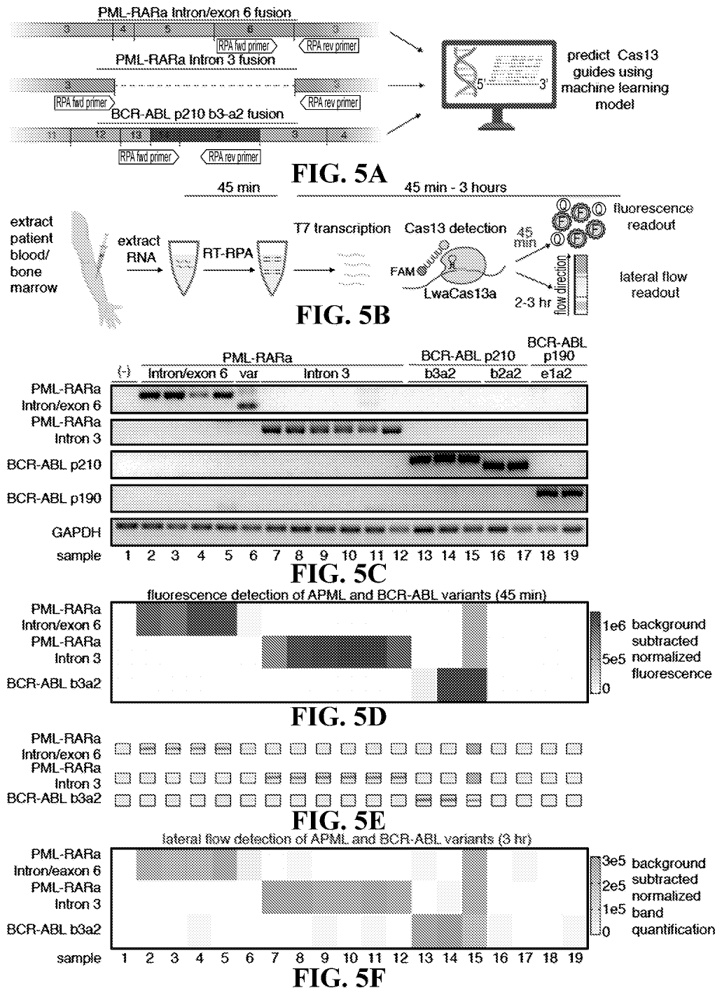

In an aspect, the cancer is selected from acute promyelocytic leukemia (APML), chronic myeloid leukemia (CIVIL), and/or acute lymphoblastic leukemia (ALL). The target in some aspects is referred to herein as a short APML or a long APML target which refers to transcripts from the long and short isoforms of the PML/RARA fusion associated with acute promyelocytic leukemia (APML). The guides may be directed to PML-RARa Intron/exon 6 fusion, PML-RARa Intron 3 fusion, and/or BCR-ABL p210 b3a2 fusion. The BCR-ABL fusion results from a reciprocal balanced translocation between chromosomes 9 and 22. Identification of these fusion variants is critical for diagnosing APL, CML, and ALL. APML with PML-RARa is a variant type of acute myeloid leukemia (AML) that is primarily associated with the t(15;17)(q22;q11-12) translocation. The fusion gene BCR-ABL1, attributed to the t(9;22) translocation, is associated with CML. In embodiments, the BCR-ABL fusion is the BCR-ABL p210 b3a2 fusion, b2a2 fusion, or a p190 ela2 fusion. See, Pane et al., Oncogene (2002) 21, 8652-8667; Ayatollahi et al, Caspian J Intern Med. 2018, 9(1):65-70; doi:10.22088/cjim.9.1.65. In an aspect, the methods disclosed herein utilize optimized guide RNAs developed with the machine learning model disclosed herein. In certain embodiments, the optimized guide RNAs can detect primary variants of the PML-RARA fusion transcript associated with t(15;17) in acute promyelocytic leukemia (APL), and a common variant of the BCR-ABL oncogene fusion transcript of chronic myeloid leukemia (CIVIL) and a subset of patients with acute lymphoblastic leukemia (ALL) (FIG. 5A-5F). BCR-ABL transcript

One or more cancers can be detected via cancer fusion genes in a multiplexing approach. Provided herein are engineered polynucleotide sequences that can direct the activity of a CRISPR protein to multiple targets using a single crRNA. The engineered polynucleotide sequences, also referred to as a multiplexing polynucleotides, can include two or more direct repeats interspersed with two or more guide sequences. More specifically, the engineered polynucleotide sequences can include a direct repeat sequence having one or more mutations relative to the corresponding wild type direct repeat sequence. The engineered polynucleotide can be configured, for example, as: 5′ DR1-G1-DR2-G2 3′. In some embodiments, the engineered polynucleotide can be configured to include three, four, five, or more additional direct repeat and guide sequences, for example: 5′ DR1-G1-DR2-G2-DR3-G3 3′, 5″ DR1-G1-DR2-G2-DR3-G3-DR4-G4 3′, or 5′ DR1-G1-DR2-G2-DR3-G3-DR4-G4-DR5-G5 3′.

Regardless of the number of direct repeat sequences, the direct repeat sequences differ from one another. Thus, DRl can be a wild type sequence and DR2 can include one or more mutations relative to the wild type sequence in accordance with the disclosure provided herein regarding direct repeats for Cas orthologs. The guide sequences can also be the same or different. In some embodiments, the guide sequences can bind to different nucleic acid targets, for example, nucleic acids encoding different polypeptides. The multiplexing polynucleotides can be as described, for example, at [0039]-[0072] in U.S. Application 62/780,748 entitled “CRISPR Cpf1 Direct Repeat Variants” and filed Dec. 17, 2018, see also U.S. Ser. No. 16/718,155, each of which is incorporated herein in its entirety by reference.

### Guide Modifications

In certain embodiments, guides of the invention comprise non-naturally occurring nucleic acids and/or non-naturally occurring nucleotides and/or nucleotide analogs, and/or chemical modifications. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, boranophosphate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, 2-thiouridine analogs, N6-methyladenosine analogs, or 2′-fluoro analogs. Further examples of modified bases include, but are not limited to, 2-aminopurine, 5-bromo-uridine, pseudouridine (Ψ), N1-methylpseudouridine (me1Ψ), 5-methoxyuridine(5moU), inosine, 7-methylguanosine. Examples of guide RNA chemical modifications include, without limitation, incorporation of 2′-0-methyl (M), 2′-O-methyl-3′-phosphorothioate (MS), phosphorothioate (PS), S-constrained ethyl(cEt), or 2′-O-methyl-3′-thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015; Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target DNA and one or more deoxyribonucleotides and/or nucleotide analogs in a region that binds to Cas9, Cpf1, or C2c1. In an embodiment of the invention, deoxyribonucleotides and/or nucleotide analogs are incorporated in engineered guide structures, such as, without limitation, 5′ and/or 3′ end, stem-loop regions, and the seed region. In certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl-3′-phosphorothioate (MS), S-constrained ethyl(cEt), or 2′-O-methyl-3′-thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554).

In certain embodiments, the CRISPR system as provided herein can make use of a crRNA or analogous polynucleotide comprising a guide sequence, wherein the polynucleotide is an RNA, a DNA or a mixture of RNA and DNA, and/or wherein the polynucleotide comprises one or more nucleotide analogs. The sequence can comprise any structure, including but not limited to a structure of a native crRNA, such as a bulge, a hairpin or a stem loop structure. In certain embodiments, the polynucleotide comprising the guide sequence forms a duplex with a second polynucleotide sequence which can be an RNA or a DNA sequence.

In certain embodiments, use is made of chemically modified guide RNAs. Examples of guide RNA chemical modifications include, without limitation, incorporation of 2′-O-methyl (M), 2′-O-methyl 3′phosphorothioate (MS), or 2′-O-methyl 3′thioPACE (MSP) at one or more terminal nucleotides. Such chemically modified guide RNAs can comprise increased stability and increased activity as compared to unmodified guide RNAs, though on-target vs. off-target specificity is not predictable. (See, Hendel, 2015, Nat Biotechnol. 33(9):985-9, doi: 10.1038/nbt.3290, published online 29 June 2015). Chemically modified guide RNAs further include, without limitation, RNAs with phosphorothioate linkages and locked nucleic acid (LNA) nucleotides comprising a methylene bridge between the 2′ and 4′ carbons of the ribose ring.

In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10 to 30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay. Similarly, cleavage of a target RNA may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N1-methylpseudouridine (me1Ψ), 5-methoxyuridine(5moU), inosine, 7-methylguanosine, 2′ -O-methyl-3′-phosphorothioate (MS), S-constrained ethyl(cEt), phosphorothioate (PS), or 2′-O-methyl-3′-thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 or 10 nucleotides in the 3′ -terminus are chemically modified. Such chemical modifications at the 3′-terminus of the Cpf1 CrRNA improve gene cutting efficiency (see Li, et al., Nature Biomedical Engineering, 2017, 1:0066). In a specific embodiment, 5 nucleotides in the 3′-terminus are replaced with 2′-fluoro analogues. In a specific embodiment, 10 nucleotides in the 3′-terminus are replaced with 2′-fluoro analogues. In a specific embodiment, 5 nucleotides in the 3′-terminus are replaced with 2′-O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the loop comprises 3, 4, or 5 nucleotides. In certain embodiments, the loop comprises the sequence of UCUU, UUUU, UAUU, or UGUU.

A guide sequence, and hence a nucleic acid-targeting guide RNA may be selected to target any target nucleic acid sequence. In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell. The target sequence may be DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nuclear RNA (snoRNA), double stranded RNA (dsRNA), non-coding RNA (ncRNA), long non-coding RNA (lncRNA), and small cytoplasmic RNA (scRNA). In some preferred embodiments, the target sequence may be a sequence within a RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

In certain embodiments, the spacer length of the guide RNA is less than 28 nucleotides. In certain embodiments, the spacer length of the guide RNA is at least 18 nucleotides and less than 28 nucleotides. In certain embodiments, the spacer length of the guide RNA is between 19 and 28 nucleotides. In certain embodiments, the spacer length of the guide RNA is between 19 and 25 nucleotides. In certain embodiments, the spacer length of the guide RNA is 20 nucleotides. In certain embodiments, the spacer length of the guide RNA is 23 nucleotides. In certain embodiments, the spacer length of the guide RNA is 25 nucleotides.

In certain embodiments, modulations of cleavage efficiency can be exploited by introduction of mismatches, e.g. 1 or more mismatches, such as 1 or 2 mismatches between spacer sequence and target sequence, including the position of the mismatch along the spacer/target. The more central (i.e. not 3′ or 5′) for instance a double mismatch is, the more cleavage efficiency is affected. Accordingly, by choosing mismatch position along the spacer, cleavage efficiency can be modulated. By means of example, if less than 100% cleavage of targets is desired (e.g. in a cell population), 1 or more, such as preferably 2 mismatches between spacer and target sequence may be introduced in the spacer sequences. The more central along the spacer of the mismatch position, the lower the cleavage percentage.

In certain example embodiments, the cleavage efficiency may be exploited to design single guides that can distinguish two or more targets that vary by a single nucleotide, such as a single nucleotide polymorphism (SNP), variation, or (point) mutation. The CRISPR effector may have reduced sensitivity to SNPs (or other single nucleotide variations) and continue to cleave SNP targets with a certain level of efficiency. Thus, for two targets, or a set of targets, a guide RNA may be designed with a nucleotide sequence that is complementary to one of the targets i.e. the on-target SNP. The guide RNA is further designed to have a synthetic mismatch. As used herein a “synthetic mismatch” refers to a non-naturally occurring mismatch that is introduced upstream or downstream of the naturally occurring SNP, such as at most 5 nucleotides upstream or downstream, for instance 4, 3, 2, or 1 nucleotide upstream or downstream, preferably at most 3 nucleotides upstream or downstream, more preferably at most 2 nucleotides upstream or downstream, most preferably 1 nucleotide upstream or downstream (i.e. adjacent the SNP). When the CRISPR effector binds to the on-target SNP, only a single mismatch will be formed with the synthetic mismatch and the CRISPR effector will continue to be activated and a detectable signal produced. When the guide RNA hybridizes to an off-target SNP, two mismatches will be formed, the mismatch from the SNP and the synthetic mismatch, and no detectable signal generated. Thus, the systems disclosed herein may be designed to distinguish SNPs within a population. For, example the systems may be used to distinguish pathogenic strains that differ by a single SNP or detect certain disease specific SNPs, such as but not limited to, disease associated SNPs, such as without limitation cancer associated SNPs.

In certain embodiments, the guide RNA is designed such that the SNP is located on position 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the spacer sequence (starting at the 5′ end). In certain embodiments, the guide RNA is designed such that the SNP is located on position 1, 2, 3, 4, 5, 6, 7, 8, or 9 of the spacer sequence (starting at the 5′ end). In certain embodiments, the guide RNA is designed such that the SNP is located on position 2, 3, 4, 5, 6, or 7of the spacer sequence (starting at the 5′ end). In certain embodiments, the guide RNA is designed such that the SNP is located on position 3, 4, 5, or 6 of the spacer sequence (starting at the 5′ end). In certain embodiments, the guide RNA is designed such that the SNP is located on position 3 of the spacer sequence (starting at the 5′ end).

In certain embodiments, the guide RNA is designed such that the mismatch (e.g. the synthetic mismatch, i.e. an additional mutation besides a SNP) is located on position 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the spacer sequence (starting at the 5′ end). In certain embodiments, the guide RNA is designed such that the mismatch is located on position 1, 2, 3, 4, 5, 6, 7, 8, or 9 of the spacer sequence (starting at the 5′ end). In certain embodiments, the guide RNA is designed such that the mismatch is located on position 4, 5, 6, or 7 of the spacer sequence (starting at the 5′ end. In certain embodiments, the guide RNA is designed such that the mismatch is located at position 3, 4, 5, or 6 of the spacer, preferably position 3. In certain embodiments, the guide RNA is designed such that the mismatch is located on position 5 of the spacer sequence (starting at the 5′ end).

In certain embodiments, said mismatch is 1, 2, 3, 4, or 5 nucleotides upstream or downstream, preferably 2 nucleotides, preferably downstream of said SNP or other single nucleotide variation in said guide RNA.

In certain embodiments, the guide RNA is designed such that the mismatch is located 2 nucleotides upstream of the SNP (i.e. one intervening nucleotide).

In certain embodiments, the guide RNA is designed such that the mismatch is located 2 nucleotides downstream of the SNP (i.e. one intervening nucleotide).

In certain embodiments, the guide RNA is designed such that the mismatch is located on position 5 of the spacer sequence (starting at the 5′ end) and the SNP is located on position 3 of the spacer sequence (starting at the 5′ end).

In certain embodiments, the guide RNA comprises a spacer which is truncated relative to a wild type spacer. In certain embodiments, the guide RNA comprises a spacer which comprises less than 28 nucleotides, preferably between and including 20 to 27 nucleotides.

In certain embodiments, the guide RNA comprises a spacer which consists of 20-25 nucleotides or 20-23 nucleotides, such as preferably 20 or 23 nucleotides.

In certain embodiments, the one or more guide RNAs are designed to detect a single nucleotide polymorphism in a target RNA or DNA, or a splice variant of an RNA transcript.

In certain embodiments, the one or more guide RNAs may be designed to bind to one or more target molecules that are diagnostic for a disease state. In some embodiments, the disease may be cancer. In some embodiments, the disease state may be an autoimmune disease. In some embodiments, the disease state may be an infection. In some embodiments, the infection may be caused by a virus, a bacterium, a fungus, a protozoa, or a parasite. In specific embodiments, the infection is a viral infection. In specific embodiments, the viral infection is caused by a DNA virus.

The embodiments described herein comprehend inducing one or more nucleotide modifications in a eukaryotic cell (in vitro, i.e. in an isolated eukaryotic cell) as herein discussed comprising delivering to cell a vector as herein discussed. The mutation(s) can include the introduction, deletion, or substitution of one or more nucleotides at each target sequence of cell(s) via the guide(s) RNA(s). The mutations can include the introduction, deletion, or substitution of 1-75 nucleotides at each target sequence of said cell(s) via the guide(s) RNA(s). The mutations can include the introduction, deletion, or substitution of 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s). The mutations can include the introduction, deletion, or substitution of 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) . The mutations include the introduction, deletion, or substitution of 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s). The mutations can include the introduction, deletion, or substitution of 20, 21, 22, 23, 24, 25, 26, 27, 28, 29, 30, 35, 40, 45, 50, or 75 nucleotides at each target sequence of said cell(s) via the guide(s) RNA(s). The mutations can include the introduction, deletion, or substitution of 40, 45, 50, 75, 100, 200, 300, 400 or 500 nucleotides at each target sequence of said cell(s) via the guide(s) RNA(s).

Typically, in the context of an endogenous CRISPR system, formation of a CRISPR complex (comprising a guide sequence hybridized to a target sequence and complexed with one or more Cas proteins) results in cleavage in or near (e.g. within 1, 2, 3, 4, 5, 6, 7, 8, 9, 10, 20, 50, or more base pairs from) the target sequence, but may depend on for instance secondary structure, in particular in the case of RNA targets. In certain embodiments, the orthologs may comprise one or more orthologs Alicyclobacillus macrosporangiidus strain DSM 17980, Bacillus hisashii strain C4, Candidatus Lindowbacteria bacterium RIFCSPLOWO2, Elusimicrobia bacterium RIFOXYA12, Omnitrophica WOR_2 bacterium RIFCSPHIGHO2, Phycisphaerae bacterium ST-NAGAB-D1, Planctomycetes bacterium RBG_13_46_10,) Spirochaetes bacterium GWB1_27_13, Verrucomicrobiaceae bacterium UBA2429(.

### Optimized Guides

A method for designing guide RNAs for use in the detection systems may comprise the steps of designing putative guide RNAs tiled across a target molecule of interest; creating a training model based on results of incubating guide RNAs with a Cas13 protein and the target molecule; predicting highly active guide RNAs for the target molecule, wherein the predicting comprises optimizing the nucleotide at each base position in the guide RNA based on the training model; and validating the predicted highly active guide RNAs by incubating the guide RNAs with the Cas13 protein and the target molecule.

In certain instances, the optimized guide for the target molecule is generated by pooling a set of guides, the guides produced by tiling guides across the target molecule; incubating the set of guides with a Cas polypeptide and the target molecule and measuring cleavage activity of each guide in the set; creating a training model based on the cleavage activity of the set of guides in the incubating step. Steps of predicting highly active guides for the target molecule and identifying the optimized guides by incubating the predicted highly active guides with the Cas polypeptide and the target molecule and selecting optimized guides may also be utilized in generating optimized guides. In embodiments, the training model comprises one or more input features selected from guide sequence, flanking target sequence, normalized positions of the guide in the target and guide GC content. In certain instances, the guide sequence and/or flanking sequence input comprises one hit encoding mono-nucleotide and/or dinucleotide In an embodiments, the training model comprises applying logistic regression model on the activity of the guides across the one or more input features.

In an aspect, the predicting highly active guides for the target molecule comprises selecting guides with an increase in activity of a guide relative to the median activity, or selecting guides with highest guide activity. In certain instances, the increase in activity is measured by an increase in fluorescence. Guides may be selected based on a particular cutoff, in certain instances based on activity relative to a median or above a particular cutoff-, for instance, are selected with a 1.5, 2, 2.5 or 3-fold activity relative to median, or are in the top quartile or quintile for each target tested.

In particular embodiments, the target is a fusion cancer target. In embodiments, one or more cancers are detected, in an aspect, the cancer is selected from acute promyelocytic leukemia (APML), chronic myeloid leukemia (CIVIL), and/or acute lymphoblastic leukemia (ALL). The target in some aspects is a short APML or a long APML target. In embodiments, transcripts from the long and short isoforms of the PML/RARA fusion associated with acute promyelocytic leukemia (APML) are targeted. The guides may be directed to PML-RARa Intron/exon 6 fusion, PML-RARa Intron 3 fusion, and/or BCR-ABL p210 b3a2 fusion. In embodiments, the BCR-ABL fusion is the BCR-ABL p210 b3a2 fusion, b2a2 fusion, or a p190 e1a2 fusion.

The optimized guides may be generated for a Cas13 ortholog. In some instances, the optimized guide is generated for a Leptotrichia wadei (Lwa) Cas13a or a Capnocytophaga canimorsus Cc5 (Cca) Cas13b ortholog.

In some embodiments, the invention provides a method for designing guide RNAs for use in the detection systems described herein. The method may comprise designing putative guide RNAs tiled across a target molecule of interest. The method may further comprise creating a training model based on results of incubating guide RNAs with a Cas13 protein and the target molecule. The method may further comprise predicting highly active guide RNAs for the target molecule. Predicting may comprise optimizing the nucleotide at each base position in the guide RNA based on the training model. The method may further comprise validating the predicted highly active guide RNAs by incubating the guide RNAs with the Cas13 protein and the target molecule.

The design of putative guide RNAs for target molecules of interest is described elsewhere herein.

The creation of training models is known in the art. Machine learning can be generalized as the ability of a learning machine to perform accurately on new, unseen examples/tasks after having experienced a learning data set. Machine learning may include the following concepts and methods. Supervised learning concepts may include AODE; Artificial neural network, such as Backpropagation, Autoencoders, Hopfield networks, Boltzmann machines, Restricted Boltzmann Machines, and Spiking neural networks; Bayesian statistics, such as Bayesian network and Bayesian knowledge base; Case-based reasoning; Gaussian process regression; Gene expression programming; Group method of data handling (GMDH); Inductive logic programming; Instance-based learning; Lazy learning; Learning Automata; Learning Vector Quantization; Logistic Model Tree; Minimum message length (decision trees, decision graphs, etc.), such as Nearest Neighbor Algorithm and Analogical modeling; Probably approximately correct learning (PAC) learning; Ripple down rules, a knowledge acquisition methodology; Symbolic machine learning algorithms; Support vector machines; Random Forests; Ensembles of classifiers, such as Bootstrap aggregating (bagging) and Boosting (meta-algorithm); Ordinal classification; Information fuzzy networks (IFN); Conditional Random Field; ANOVA; Linear classifiers, such as Fisher's linear discriminant, Linear regression, Logistic regression, Multinomial logistic regression, Naive Bayes classifier, Perceptron, Support vector machines; Quadratic classifiers; k-nearest neighbor; Boosting; Decision trees, such as C4.5, Random forests, ID3, CART, SLIQ, SPRINT; Bayesian networks, such as Naive Bayes; and Hidden Markov models. Unsupervised learning concepts may include; Expectation-maximization algorithm; Vector Quantization; Generative topographic map; Information bottleneck method; Artificial neural network, such as Self-organizing map; Association rule learning, such as, Apriori algorithm, Eclat algorithm, and FP-growth algorithm; Hierarchical clustering, such as Single-linkage clustering and Conceptual clustering; Cluster analysis, such as, K-means algorithm, Fuzzy clustering, DBSCAN, and OPTICS algorithm; and Outlier Detection, such as Local Outlier Factor. Semi-supervised learning concepts may include; Generative models; Low-density separation; Graph-based methods; and Co-training. Reinforcement learning concepts may include; Temporal difference learning; Q-learning; Learning Automata; and SARSA. Deep learning concepts may include; Deep belief networks; Deep Boltzmann machines; Deep Convolutional neural networks; Deep Recurrent neural networks; and Hierarchical temporal memory.

The methods as disclosed herein designing putative guide molecules may comprise design based on one or more variables, including guide sequence, flanking target sequence, guide position and guide GC content as input features. In certain embodiments, the length of the flanking target region can be considered a free parameter and can be further selected during cross-validation. Additionally, mono-nucleotide and/or dinucleotide based identities across a guide length and flanking sequence in the target, varying one or more of flanking sequence length, normalized positions of the guide in the target, and GC content of the guide, or a combination thereof.

In embodiments, the training model for the guide design is Cas protein specific. In embodiments, the Cas protein is a Cas13a, Cas13b or Cas12 a protein. In certain embodiments, the protein is LwaCas13a or CcaCas13b. Selection for the best guides can be dependent on each enzyme. In particular embodiments, where majority of guides have activity above background on a per-target basis, selection of guides may be based on 1.5 fold, 2, 2.5, 3 or more fold activity over the median activity. In other instances, the best performing guides may be at or near background fluorescence. In this instance, the guide selection may be based on a top percentile, e.g. quartile or quintile, of performing guides.

Codon optimization is described elsewhere herein. In specific embodiments, the nucleotide at each base position in the guide RNA may be optimized based on the training model, thus allowing for prediction of highly active guide RNAs for the target molecule. In certain instances, mono-nucleotide and/or dinucleotide based identities across a guide length and flanking sequence in the target may be optimized.

The predicted highly active guide RNAs may then be validated or verified by incubating the guide molecules with a Cas polypeptide, such as Cas13 protein and the target molecule, as described in the examples.

In certain embodiments, optimization comprises validation of best performing models for a particular Cas polypeptide across multiple guides may comprise comparing the predicted score of each guide versus actual collateral activity upon target recognition. In embodiments, kinetic data of the best and worst predicted guides are evaluated. In embodiments, lateral flow performance of the predicted guides is evaluated for a target sequence.

### Detection Constructs

As used herein, a “detection construct” refers to a molecule that can be cleaved or otherwise deactivated by an activated CRISPR system effector protein described herein. The term “detection construct” may also be referred to in the alternative as a “masking construct.” Depending on the nuclease activity of the CRISPR effector protein, the masking construct may be a RNA-based masking construct or a DNA-based masking construct. The Nucleic Acid-based masking constructs comprises a nucleic acid element that is cleavable by a CRISPR effector protein. Cleavage of the nucleic acid element releases agents or produces conformational changes that allow a detectable signal to be produced. Example constructs demonstrating how the nucleic acid element may be used to prevent or mask generation of detectable signal are described below and embodiments of the invention comprise variants of the same. Prior to cleavage, or when the masking construct is in an ‘active’ state, the masking construct blocks the generation or detection of a positive detectable signal. It will be understood that in certain example embodiments a minimal background signal may be produced in the presence of an active masking construct. A positive detectable signal may be any signal that can be detected using optical, fluorescent, chemiluminescent, electrochemical or other detection methods known in the art. The term “positive detectable signal” is used to differentiate from other detectable signals that may be detectable in the presence of the masking construct. For example, in certain embodiments a first signal may be detected when the masking agent is present or when a CRISPR system has not been activated (i.e. a negative detectable signal), which then converts to a second signal (e.g. the positive detectable signal) upon detection of the target molecules and cleavage or deactivation of the masking agent, or upon activation of the CRISPR effector protein. The positive detectable signal, then, is a signal detected upon activation of the CRISPR effector protein, and may be, in a colorimetric or fluorescent assay, a decrease in fluorescence or color relative to a control or an increase in fluorescence or color relative to a control, depending on the configuration of the lateral flow substrate, and as described further herein.

In certain example embodiments, the masking construct may comprise a HCR initiator sequence and a cutting motif, or a cleavable structural element, such as a loop or hairpin, that prevents the initiator from initiating the HCR reaction. The cutting motif may be preferentially cut by one of the activated CRISPR effector proteins. Upon cleavage of the cutting motif or structure element by an activated CRISPR effector protein, the initiator is then released to trigger the HCR reaction, detection thereof indicating the presence of one or more targets in the sample. In certain example embodiments, the masking construct comprises a hairpin with a RNA loop. When an activated CRISPR effector protein cuts the RNA loop, the initiator can be released to trigger the HCR reaction.

In certain example embodiments, the masking construct may suppress generation of a gene product. The gene product may be encoded by a reporter construct that is added to the sample. The masking construct may be an interfering RNA involved in a RNA interference pathway, such as a short hairpin RNA (shRNA) or small interfering RNA (siRNA). The masking construct may also comprise microRNA (miRNA). While present, the masking construct suppresses expression of the gene product. The gene product may be a fluorescent protein or other RNA transcript or proteins that would otherwise be detectable by a labeled probe, aptamer, or antibody but for the presence of the masking construct. Upon activation of the effector protein the masking construct is cleaved or otherwise silenced allowing for expression and detection of the gene product as the positive detectable signal.

In specific embodiments, the masking construct comprises a silencing RNA that suppresses generation of a gene product encoded by a reporting construct, wherein the gene product generates the detectable positive signal when expressed.

In certain example embodiments, the masking construct may sequester one or more reagents needed to generate a detectable positive signal such that release of the one or more reagents from the masking construct results in generation of the detectable positive signal. The one or more reagents may combine to produce a colorimetric signal, a chemiluminescent signal, a fluorescent signal, or any other detectable signal and may comprise any reagents known to be suitable for such purposes. In certain example embodiments, the one or more reagents are sequestered by RNA aptamers that bind the one or more reagents. The one or more reagents are released when the effector protein is activated upon detection of a target molecule and the RNA or DNA aptamers are degraded.

In certain example embodiments, the masking construct may be immobilized on a solid substrate in an individual discrete volume (defined further below) and sequesters a single reagent. For example, the reagent may be a bead comprising a dye. When sequestered by the immobilized reagent, the individual beads are too diffuse to generate a detectable signal, but upon release from the masking construct are able to generate a detectable signal, for example by aggregation or simple increase in solution concentration. In certain example embodiments, the immobilized masking agent is a RNA- or DNA-based aptamer that can be cleaved by the activated effector protein upon detection of a target molecule.

In certain other example embodiments, the masking construct binds to an immobilized reagent in solution thereby blocking the ability of the reagent to bind to a separate labeled binding partner that is free in solution. Thus, upon application of a washing step to a sample, the labeled binding partner can be washed out of the sample in the absence of a target molecule. However, if the effector protein is activated, the masking construct is cleaved to a degree sufficient to interfere with the ability of the masking construct to bind the reagent thereby allowing the labeled binding partner to bind to the immobilized reagent. Thus, the labeled binding partner remains after the wash step indicating the presence of the target molecule in the sample. In certain aspects, the masking construct that binds the immobilized reagent is a DNA or RNA aptamer. The immobilized reagent may be a protein and the labeled binding partner may be a labeled antibody. Alternatively, the immobilized reagent may be streptavidin and the labeled binding partner may be labeled biotin. The label on the binding partner used in the above embodiments may be any detectable label known in the art. In addition, other known binding partners may be used in accordance with the overall design described herein.

In certain example embodiments, the masking construct may comprise a ribozyme. Ribozymes are RNA molecules having catalytic properties. Ribozymes, both naturally and engineered, comprise or consist of RNA that may be targeted by the effector proteins disclosed herein. The ribozyme may be selected or engineered to catalyze a reaction that either generates a negative detectable signal or prevents generation of a positive control signal. Upon deactivation of the ribozyme by the activated effector protein, the reaction generating a negative control signal, or preventing generation of a positive detectable signal, is removed thereby allowing a positive detectable signal to be generated. In one example embodiment, the ribozyme may catalyze a colorimetric reaction causing a solution to appear as a first color. When the ribozyme is deactivated the solution then turns to a second color, the second color being the detectable positive signal. An example of how ribozymes can be used to catalyze a colorimetric reaction are described in Zhao et al. “Signal amplification of glucosamine-6-phosphate based on ribozyme glmS,” Biosens Bioelectron. 2014; 16:337-42, and provide an example of how such a system could be modified to work in the context of the embodiments disclosed herein. Alternatively, ribozymes, when present can generate cleavage products of, for example, RNA transcripts. Thus, detection of a positive detectable signal may comprise detection of non-cleaved RNA transcripts that are only generated in the absence of the ribozyme.

In some embodiments, the masking construct may be a ribozyme that generates a negative detectable signal, and wherein a positive detectable signal is generated when the ribozyme is deactivated.

In certain example embodiments, the one or more reagents is a protein, such as an enzyme, capable of facilitating generation of a detectable signal, such as a colorimetric, chemiluminescent, or fluorescent signal, that is inhibited or sequestered such that the protein cannot generate the detectable signal by the binding of one or more DNA or RNA aptamers to the protein. Upon activation of the effector proteins disclosed herein, the DNA or RNA aptamers are cleaved or degraded to an extent that they no longer inhibit the protein's ability to generate the detectable signal. In certain example embodiments, the aptamer is a thrombin inhibitor aptamer. In certain example embodiments the thrombin inhibitor aptamer has a sequence of GGGAACAAAGCUGAAGUACUUACCC (SEQ ID NO: 8). When this aptamer is cleaved, thrombin will become active and will cleave a peptide colorimetric or fluorescent substrate. In certain example embodiments, the colorimetric substrate is para-nitroanilide (pNA) covalently linked to the peptide substrate for thrombin. Upon cleavage by thrombin, pNA is released and becomes yellow in color and easily visible to the eye. In certain example embodiments, the fluorescent substrate is 7-amino-4-methylcoumarin a blue fluorophore that can be detected using a fluorescence detector. Inhibitory aptamers may also be used for horseradish peroxidase (HRP), beta-galactosidase, or calf alkaline phosphatase (CAP) and within the general principals laid out above.

In certain embodiments, RNAse or DNAse activity is detected colorimetrically via cleavage of enzyme-inhibiting aptamers. One potential mode of converting DNAse or RNAse activity into a colorimetric signal is to couple the cleavage of a DNA or RNA aptamer with the re-activation of an enzyme that is capable of producing a colorimetric output. In the absence of RNA or DNA cleavage, the intact aptamer will bind to the enzyme target and inhibit its activity. The advantage of this readout system is that the enzyme provides an additional amplification step: once liberated from an aptamer via collateral activity (e.g. Cpf1 collateral activity), the colorimetric enzyme will continue to produce colorimetric product, leading to a multiplication of signal.

In certain embodiments, an existing aptamer that inhibits an enzyme with a colorimetric readout is used. Several aptamer/enzyme pairs with colorimetric readouts exist, such as thrombin, protein C, neutrophil elastase, and subtilisin. These proteases have colorimetric substrates based upon pNA and are commercially available. In certain embodiments, a novel aptamer targeting a common colorimetric enzyme is used. Common and robust enzymes, such as beta-galactosidase, horseradish peroxidase, or calf intestinal alkaline phosphatase, could be targeted by engineered aptamers designed by selection strategies such as SELEX. Such strategies allow for quick selection of aptamers with nanomolar binding efficiencies and could be used for the development of additional enzyme/aptamer pairs for colorimetric readout.

In certain embodiments, the masking construct may be a DNA or RNA aptamer and/or may comprise a DNA or RNA-tethered inhibitor.

In certain embodiments, the masking construct may comprise a DNA or RNA oligonucleotide to which a detectable ligand and a masking component are attached.

In certain embodiments, RNAse or DNase activity is detected colorimetrically via cleavage of RNA-tethered inhibitors. Many common colorimetric enzymes have competitive, reversible inhibitors: for example, beta-galactosidase can be inhibited by galactose. Many of these inhibitors are weak, but their effect can be increased by increases in local concentration. By linking local concentration of inhibitors to DNase RNAse activity, colorimetric enzyme and inhibitor pairs can be engineered into DNase and RNAse sensors. The colorimetric DNase or RNAse sensor based upon small-molecule inhibitors involves three components: the colorimetric enzyme, the inhibitor, and a bridging RNA or DNA that is covalently linked to both the inhibitor and enzyme, tethering the inhibitor to the enzyme. In the uncleaved configuration, the enzyme is inhibited by the increased local concentration of the small molecule; when the DNA or RNA is cleaved (e.g. by Cas13 or Cas12 collateral cleavage), the inhibitor will be released and the colorimetric enzyme will be activated.

In certain embodiments, the aptamer or DNA- or RNA-tethered inhibitor may sequester an enzyme, wherein the enzyme generates a detectable signal upon release from the aptamer or DNA or RNA tethered inhibitor by acting upon a substrate. In some embodiments, the aptamer may be an inhibitor aptamer that inhibits an enzyme and prevents the enzyme from catalyzing generation of a detectable signal from a substance. In some embodiments, the DNA- or RNA-tethered inhibitor may inhibit an enzyme and may prevent the enzyme from catalyzing generation of a detectable signal from a substrate.

In certain embodiments, RNAse activity is detected colorimetrically via formation and/or activation of G-quadruplexes. G quadruplexes in DNA can complex with heme (iron (III)-protoporphyrin IX) to form a DNAzyme with peroxidase activity. When supplied with a peroxidase substrate (e.g. ABTS: (2,2′-Azinobis [3-ethylbenzothiazoline-6-sulfonic acid]-diammonium salt)), the G-quadruplex-heme complex in the presence of hydrogen peroxide causes oxidation of the substrate, which then forms a green color in solution. An example G-quadruplex forming DNA sequence is: GGGTAGGGCGGGTTGGGA (SEQ ID NO: 9). By hybridizing an additional DNA or RNA sequence, referred to herein as a “staple,” to this DNA aptamer, formation of the G-quadraplex structure will be limited. Upon collateral activation, the staple will be cleaved allowing the G quadraplex to form and heme to bind. This strategy is particularly appealing because color formation is enzymatic, meaning there is additional amplification beyond collateral activation.

In certain embodiments, the masking construct may comprise an RNA oligonucleotide designed to bind a G-quadruplex forming sequence, wherein a G-quadruplex structure is formed by the G-quadruplex forming sequence upon cleavage of the masking construct, and wherein the G-quadruplex structure generates a detectable positive signal.

In certain example embodiments, the masking construct may be immobilized on a solid substrate in an individual discrete volume (defined further below) and sequesters a single reagent. For example, the reagent may be a bead comprising a dye. When sequestered by the immobilized reagent, the individual beads are too diffuse to generate a detectable signal, but upon release from the masking construct are able to generate a detectable signal, for example by aggregation or simple increase in solution concentration. In certain example embodiments, the immobilized masking agent is a DNA- or RNA-based aptamer that can be cleaved by the activated effector protein upon detection of a target molecule.

In one example embodiment, the masking construct comprises a detection agent that changes color depending on whether the detection agent is aggregated or dispersed in solution. For example, certain nanoparticles, such as colloidal gold, undergo a visible purple to red color shift as they move from aggregates to dispersed particles. Accordingly, in certain example embodiments, such detection agents may be held in aggregate by one or more bridge molecules. At least a portion of the bridge molecule comprises RNA or DNA. Upon activation of the effector proteins disclosed herein, the RNA or DNA portion of the bridge molecule is cleaved allowing the detection agent to disperse and resulting in the corresponding change in color. In certain example embodiments, the detection agent is a colloidal metal. The colloidal metal material may include water-insoluble metal particles or metallic compounds dispersed in a liquid, a hydrosol, or a metal sol. The colloidal metal may be selected from the metals in groups IA, IB, IIB and IIIB of the periodic table, as well as the transition metals, especially those of group VIII. Preferred metals include gold, silver, aluminum, ruthenium, zinc, iron, nickel and calcium. Other suitable metals also include the following in all of their various oxidation states: lithium, sodium, magnesium, potassium, scandium, titanium, vanadium, chromium, manganese, cobalt, copper, gallium, strontium, niobium, molybdenum, palladium, indium, tin, tungsten, rhenium, platinum, and gadolinium. The metals are preferably provided in ionic form, derived from an appropriate metal compound, for example the A13+, Ru3+, Zn2+, Fe3+, Ni2+ and Ca2+ ions.

When the RNA or DNA bridge is cut by the activated CRISPR effector, the aforementioned color shift is observed. In certain example embodiments, the particles are colloidal metals. In certain other example embodiments, the colloidal metal is a colloidal gold. In certain example embodiments, the colloidal nanoparticles are 15 nm gold nanoparticles (AuNPs). Due to the unique surface properties of colloidal gold nanoparticles, maximal absorbance is observed at 520 nm when fully dispersed in solution and appear red in color to the naked eye. Upon aggregation of AuNPs, they exhibit a red-shift in maximal absorbance and appear darker in color, eventually precipitating from solution as a dark purple aggregate. In certain example embodiments the nanoparticles are modified to include DNA linkers extending from the surface of the nanoparticle. Individual particles are linked together by single-stranded RNA (ssRNA) or single-stranded DNA bridges that hybridize on each end to at least a portion of the DNA linkers. Thus, the nanoparticles will form a web of linked particles and aggregate, appearing as a dark precipitate. Upon activation of the CRISPR effectors disclosed herein, the ssRNA or ssDNA bridge will be cleaved, releasing the AU NPS from the linked mesh and producing a visible red color. Example DNA linkers and bridge sequences are listed below. Thiol linkers on the end of the DNA linkers may be used for surface conjugation to the AuNPS. Other forms of conjugation may be used. In certain example embodiments, two populations of AuNPs may be generated, one for each DNA linker. This will help facilitate proper binding of the ssRNA bridge with proper orientation. In certain example embodiments, a first DNA linker is conjugated by the 3′ end while a second DNA linker is conjugated by the 5′ end.

In certain other example embodiments, the masking construct may comprise an RNA or DNA oligonucleotide to which are attached a detectable label and a masking agent of that detectable label. An example of such a detectable label/masking agent pair is a fluorophore and a quencher of the fluorophore. Quenching of the fluorophore can occur as a result of the formation of a non-fluorescent complex between the fluorophore and another fluorophore or non-fluorescent molecule. This mechanism is known as ground-state complex formation, static quenching, or contact quenching. Accordingly, the RNA or DNA oligonucleotide may be designed so that the fluorophore and quencher are in sufficient proximity for contact quenching to occur. Fluorophores and their cognate quenchers are known in the art and can be selected for this purpose by one having ordinary skill in the art. The particular fluorophore/quencher pair is not critical in the context of this invention, only that selection of the fluorophore/quencher pairs ensures masking of the fluorophore. Upon activation of the effector proteins disclosed herein, the RNA or DNA oligonucleotide is cleaved thereby severing the proximity between the fluorophore and quencher needed to maintain the contact quenching effect. Accordingly, detection of the fluorophore may be used to determine the presence of a target molecule in a sample.

In certain other example embodiments, the masking construct may comprise one or more RNA oligonucleotides to which are attached one or more metal nanoparticles, such as gold nanoparticles. In some embodiments, the masking construct comprises a plurality of metal nanoparticles crosslinked by a plurality of RNA or DNA oligonucleotides forming a closed loop. In one embodiment, the masking construct comprises three gold nanoparticles crosslinked by three RNA or DNA oligonucleotides forming a closed loop. In some embodiments, the cleavage of the RNA or DNA oligonucleotides by the CRISPR effector protein leads to a detectable signal produced by the metal nanoparticles.

In certain other example embodiments, the masking construct may comprise one or more RNA or DNA oligonucleotides to which are attached one or more quantum dots. In some embodiments, the cleavage of the RNA or DNA oligonucleotides by the CRISPR effector protein leads to a detectable signal produced by the quantum dots.

In one example embodiment, the masking construct may comprise a quantum dot. The quantum dot may have multiple linker molecules attached to the surface. At least a portion of the linker molecule comprises RNA or DNA. The linker molecule is attached to the quantum dot at one end and to one or more quenchers along the length or at terminal ends of the linker such that the quenchers are maintained in sufficient proximity for quenching of the quantum dot to occur. The linker may be branched. As above, the quantum dot/quencher pair is not critical, only that selection of the quantum dot/quencher pair ensures masking of the fluorophore. Quantum dots and their cognate quenchers are known in the art and can be selected for this purpose by one having ordinary skill in the art. Upon activation of the effector proteins disclosed herein, the RNA or DNA portion of the linker molecule is cleaved thereby eliminating the proximity between the quantum dot and one or more quenchers needed to maintain the quenching effect. In certain example embodiments the quantum dot is streptavidin conjugated. RNA or DNA are attached via biotin linkers and recruit quenching molecules with the sequences /5Biosg/UCUCGUACGUUC/3IAbRQSp/ (SEQ ID NO: 10) or /5Biosg/UCUCGUACGUUCUCUCGUACGUUC/3IAbRQSp/ (SEQ ID NO: 11) where /5Biosg/ is a biotin tag and /31AbRQSp/ is an Iowa black quencher (Iowa Black FQ). Upon cleavage, by the activated effectors disclosed herein the quantum dot will fluoresce visibly.

In specific embodiments, the detectable ligand may be a fluorophore and the masking component may be a quencher molecule.

In a similar fashion, fluorescence energy transfer (FRET) may be used to generate a detectable positive signal. FRET is a non-radiative process by which a photon from an energetically excited fluorophore (i.e. “donor fluorophore”) raises the energy state of an electron in another molecule (i.e. “the acceptor”) to higher vibrational levels of the excited singlet state. The donor fluorophore returns to the ground state without emitting a fluoresce characteristic of that fluorophore. The acceptor can be another fluorophore or non-fluorescent molecule. If the acceptor is a fluorophore, the transferred energy is emitted as fluorescence characteristic of that fluorophore. If the acceptor is a non-fluorescent molecule the absorbed energy is loss as heat. Thus, in the context of the embodiments disclosed herein, the fluorophore/quencher pair is replaced with a donor fluorophore/acceptor pair attached to the oligonucleotide molecule. When intact, the masking construct generates a first signal (negative detectable signal) as detected by the fluorescence or heat emitted from the acceptor. Upon activation of the effector proteins disclosed herein the RNA oligonucleotide is cleaved and FRET is disrupted such that fluorescence of the donor fluorophore is now detected (positive detectable signal).

In certain example embodiments, the masking construct comprises the use of intercalating dyes which change their absorbance in response to cleavage of long RNAs or DNAs to short nucleotides. Several such dyes exist. For example, pyronine-Y will complex with RNA and form a complex that has an absorbance at 572 nm. Cleavage of the RNA results in loss of absorbance and a color change. Methylene blue may be used in a similar fashion, with changes in absorbance at 688 nm upon RNA cleavage. Accordingly, in certain example embodiments the masking construct comprises a RNA and intercalating dye complex that changes absorbance upon the cleavage of RNA by the effector proteins disclosed herein.

In certain example embodiments, the masking construct may comprise an initiator for an HCR reaction. See e.g. Dirks and Pierce. PNAS 101, 15275-15728 (2004). HCR reactions utilize the potential energy in two hairpin species. When a single-stranded initiator having a portion of complementary to a corresponding region on one of the hairpins is released into the previously stable mixture, it opens a hairpin of one species. This process, in turn, exposes a single-stranded region that opens a hairpin of the other species. This process, in turn, exposes a single stranded region identical to the original initiator. The resulting chain reaction may lead to the formation of a nicked double helix that grows until the hairpin supply is exhausted. Detection of the resulting products may be done on a gel or colorimetrically. Example colorimetric detection methods include, for example, those disclosed in Lu et al. “Ultra-sensitive colorimetric assay system based on the hybridization chain reaction-triggered enzyme cascade amplification ACS Appl Mater Interfaces, 2017, 9(1):167-175, Wang et al. “An enzyme-free colorimetric assay using hybridization chain reaction amplification and split aptamers” Analyst 2015, 150, 7657-7662, and Song et al. “Non covalent fluorescent labeling of hairpin DNA probe coupled with hybridization chain reaction for sensitive DNA detection.” Applied Spectroscopy, 70(4): 686-694 (2016).

In certain example embodiments, the masking construct suppresses generation of a detectable positive signal until cleaved, or modified by an activated CRISPR effector protein. In some embodiments, the masking construct may suppress generation of a detectable positive signal by masking the detectable positive signal, or generating a detectable negative signal instead.

### Samples

Samples to be screened are loaded at the sample loading portion of the lateral flow substrate. The samples must be liquid samples or samples dissolved in an appropriate solvent, usually aqueous. The liquid sample reconstitutes the SHERLOCK reagents such that a SHERLOCK reaction can occur. The liquid sample begins to flow from the sample portion of the substrate towards the first and second capture regions.

A sample for use with the invention may be a biological or environmental sample, such as a surface sample, a fluid sample, or a food sample (fresh fruits or vegetables, meats). Food samples may include a beverage sample, a paper surface, a fabric surface, a metal surface, a wood surface, a plastic surface, a soil sample, a freshwater sample, a wastewater sample, a saline water sample, exposure to atmospheric air or other gas sample, or a combination thereof. For example, household/commercial/industrial surfaces made of any materials including, but not limited to, metal, wood, plastic, rubber, or the like, may be swabbed and tested for contaminants. Soil samples may be tested for the presence of pathogenic bacteria or parasites, or other microbes, both for environmental purposes and/or for human, animal, or plant disease testing. Water samples such as freshwater samples, wastewater samples, or saline water samples can be evaluated for cleanliness and safety, and/or potability, to detect the presence of, for example, Cryptosporidium parvum, Giardia lamblia, or other microbial contamination. In further embodiments, a biological sample may be obtained from a source including, but not limited to, a tissue sample, saliva, blood, plasma, sera, stool, urine, sputum, mucous, lymph, synovial fluid, spinal fluid, cerebrospinal fluid, ascites, pleural effusion, seroma, pus, bile, aqueous or vitreous humor, transudate, exudate, or swab of skin or a mucosal membrane surface. In some particular embodiments, an environmental sample or biological samples may be crude samples and/or the one or more target molecules may not be purified or amplified from the sample prior to application of the method. Identification of microbes may be useful and/or needed for any number of applications, and thus any type of sample from any source deemed appropriate by one of skill in the art may be used in accordance with the invention.

Methods for Detecting and/or Quantifying Target Nucleic Acids

In some embodiments, the invention provides methods for detecting target nucleic acids in a sample. Such methods may comprise contacting a sample with the first end of a lateral flow device as described herein. The first end of the lateral flow device may comprise a sample loading portion, wherein the sample flows from the sample loading portion of the substrate towards the first and second capture regions and generates a detectable signal.

A positive detectable signal may be any signal that can be detected using optical, fluorescent, chemiluminescent, electrochemical or other detection methods known in the art, as described elsewhere herein.

In some embodiments, the lateral flow device may be capable of detecting two different target nucleic acid sequences. In some embodiments, this detection of two different target nucleic acid sequences may occur simultaneously.

In some embodiments, the absence of target nucleic acid sequences in a sample elicits a detectable fluorescent signal at each capture region. In such instances, the absence of any target nucleic acid sequences in a sample may cause a detectable signal to appear at the first and second capture regions.

In some embodiments, the lateral flow device as described herein is capable of detecting three different target nucleic acid sequences. In specific embodiments, when the target nucleic acid sequences are absent from the sample, a fluorescent signal may be generated at each of the three capture regions. In such exemplary embodiments, a fluorescent signal may be absent at the capture region for the corresponding target nucleic acid sequence when the sample contains one or more target nucleic acid sequences.

Samples to be screened are loaded at the sample loading portion of the lateral flow substrate. The samples must be liquid samples or samples dissolved in an appropriate solvent, usually aqueous. The liquid sample reconstitutes the system reagents such that a SHERLOCK reaction can occur. Intact reporter construct is bound at the first capture region by binding between the first binding agent and the first molecule. Likewise, the detection agent will begin to collect at the first binding region by binding to the second molecule on the intact reporter construct. If target molecule(s) are present in the sample, the CRISPR effector protein collateral effect is activated. As activated CRISPR effector protein comes into contact with the bound reporter construct, the reporter constructs are cleaved, releasing the second molecule to flow further down the lateral flow substrate towards the second binding region. The released second molecule is then captured at the second capture region by binding to the second binding agent, where additional detection agent may also accumulate by binding to the second molecule. Accordingly, if the target molecule(s) is not present in the sample, a detectable signal will appear at the first capture region, and if the target molecule(s) is present in the sample, a detectable signal will appear at the location of the second capture region.

In some embodiments, the invention provides a method for quantifying target nucleic acids in samples comprising distributing a sample or set of samples into one or more individual discrete volumes comprising two or more CRISPR systems as described herein. The method may comprise using HDA to amplify one or more target molecules in the sample or set of samples, as described herein. The method may further comprise incubating the sample or set of samples under conditions sufficient to allow binding of the guide RNAs to one or more target molecules. The method may further comprise activating the CRISPR effector protein via binding of the guide RNAs to the one or more target molecules. Activating the CRISPR effector protein may result in modification of the detection construct such that a detectable positive signal is generated. The method may further comprise detecting the one or more detectable positive signals, wherein detection indicates the presence of one or more target molecules in the sample. The method may further comprise comparing the intensity of the one or more signals to a control to quantify the nucleic acid in the sample. The steps of amplifying, incubating, activating, and detecting may all be performed in the same individual discrete volume.

### Amplifying Target Molecules

The step of amplifying one or more target molecules can comprise amplification systems known in the art. In some embodiments, amplification is isothermal. In certain example embodiments, target RNAs and/or DNAs may be amplified prior to activating the CRISPR effector protein. Any suitable RNA or DNA amplification technique may be used. In certain example embodiments, the RNA or DNA amplification is an isothermal amplification. In certain example embodiments, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In certain example embodiments, non-isothermal amplification methods may be used which include, but are not limited to, PCR, multiple displacement amplification (MDA), rolling circle amplification (RCA), ligase chain reaction (LCR), or ramification amplification method (RAM).

In certain example embodiments, the RNA or DNA amplification is NASBA, which is initiated with reverse transcription of target RNA by a sequence-specific reverse primer to create a RNA/DNA duplex. RNase H is then used to degrade the RNA template, allowing a forward primer containing a promoter, such as the T7 promoter, to bind and initiate elongation of the complementary strand, generating a double-stranded DNA product. The RNA polymerase promoter-mediated transcription of the DNA template then creates copies of the target RNA sequence. Importantly, each of the new target RNAs can be detected by the guide RNAs thus further enhancing the sensitivity of the assay. Binding of the target RNAs by the guide RNAs then leads to activation of the CRISPR effector protein and the methods proceed as outlined above. The NASBA reaction has the additional advantage of being able to proceed under moderate isothermal conditions, for example at approximately 41° C., making it suitable for systems and devices deployed for early and direct detection in the field and far from clinical laboratories.

In certain other example embodiments, a recombinase polymerase amplification (RPA) reaction may be used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In certain example embodiments, a RNA polymerase promoter, such as a T7 promoter, is added to one of the primers. This results in an amplified double-stranded DNA product comprising the target sequence and a RNA polymerase promoter. After, or during, the RPA reaction, a RNA polymerase is added that will produce RNA from the double-stranded DNA templates. The amplified target RNA can then in turn be detected by the CRISPR effector system. In this way target DNA can be detected using the embodiments disclosed herein. RPA reactions can also be used to amplify target RNA. The target RNA is first converted to cDNA using a reverse transcriptase, followed by second strand DNA synthesis, at which point the RPA reaction proceeds as outlined above. In embodiments, the RPA reaction is an RT-RPA. In one embodiment, the RT used is an AMV RT.

An embodiment of the invention may comprise nickase-based amplification. The nicking enzyme may be a CRISPR protein. Accordingly, the introduction of nicks into dsDNA can be programmable and sequence-specific. FIG. 115 depicts an embodiment of the invention, which starts with two guides designed to target opposite strands of a dsDNA target. According to the invention, the nickase can be Cpf1, C2c1, Cas9 or any ortholog or CRISPR protein that cleaves or is engineered to cleave a single strand of a DNA duplex. The nicked strands may then be extended by a polymerase. In an embodiment, the locations of the nicks are selected such that extension of the strands by a polymerase is towards the central portion of the target duplex DNA between the nick sites. In certain embodiments, primers are included in the reaction capable of hybridizing to the extended strands followed by further polymerase extension of the primers to regenerate two dsDNA pieces: a first dsDNA that includes the first strand Cpf1 guide site or both the first and second strand Cpf1 guide sites, and a second dsDNA that includes the second strand Cpf1 guide site or both the first and second strand Cprf guide sites. These pieces continue to be nicked and extended in a cyclic reaction that exponentially amplifies the region of the target between nicking sites.

The amplification can be isothermal and selected for temperature. In one embodiment, the amplification proceeds rapidly at 37 degrees. In other embodiments, the temperature of the isothermal amplification may be chosen by selecting a polymerase (e.g. Bsu, Bst, Phi29, klenow fragment etc.).operable at a different temperature.

Thus, whereas nicking isothermal amplification techniques use nicking enzymes with fixed sequence preference (e.g. in nicking enzyme amplification reaction or NEAR), which requires denaturing of the original dsDNA target to allow annealing and extension of primers that add the nicking substrate to the ends of the target, use of a CRISPR nickase wherein the nicking sites can be programed via guide RNAs means that no denaturing step is necessary, enabling the entire reaction to be truly isothermal. This also simplifies the reaction because these primers that add the nicking substrate are different than the primers that are used later in the reaction, meaning that NEAR requires two primer sets (i.e. 4 primers) while Cpf1 nicking amplification only requires one primer set (i.e. two primers). This makes nicking Cpf1 amplification much simpler and easier to operate without complicated instrumentation to perform the denaturation and then cooling to the isothermal temperature.

Accordingly, in certain example embodiments the systems disclosed herein may include amplification reagents. Different components or reagents useful for amplification of nucleic acids are described herein. For example, an amplification reagent as described herein may include a buffer, such as a Tris buffer. A Tris buffer may be used at any concentration appropriate for the desired application or use, for example including, but not limited to, a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 1 M, or the like. One of skill in the art will be able to determine an appropriate concentration of a buffer such as Tris for use with the present invention.

A salt, such as magnesium chloride (MgCl2), potassium chloride (KCl), or sodium chloride (NaCl), may be included in an amplification reaction, such as PCR, in order to improve the amplification of nucleic acid fragments. Although the salt concentration will depend on the particular reaction and application, in some embodiments, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present invention and as described herein.

Other components of a biological or chemical reaction may include a cell lysis component in order to break open or lyse a cell for analysis of the materials therein. A cell lysis component may include, but is not limited to, a detergent, a salt as described above, such as NaCl, KCl, ammonium sulfate [(NH4)2SO4], or others. Detergents that may be appropriate for the invention may include Triton X-100, sodium dodecyl sulfate (SDS), CHAPS (3-[(3-cholamidopropyl)dimethylammonio]-1-propanesulfonate), ethyl trimethyl ammonium bromide, nonyl phenoxypolyethoxylethanol (NP-40). Concentrations of detergents may depend on the particular application, and may be specific to the reaction in some cases. Amplification reactions may include dNTPs and nucleic acid primers used at any concentration appropriate for the invention, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like. Likewise, a polymerase useful in accordance with the invention may be any specific or general polymerase known in the art and useful or the invention, including Taq polymerase, Q5 polymerase, or the like.

In some embodiments, amplification reagents as described herein may be appropriate for use in hot-start amplification. Hot start amplification may be beneficial in some embodiments to reduce or eliminate dimerization of adaptor molecules or oligos, or to otherwise prevent unwanted amplification products or artifacts and obtain optimum amplification of the desired product. Many components described herein for use in amplification may also be used in hot-start amplification. In some embodiments, reagents or components appropriate for use with hot-start amplification may be used in place of one or more of the composition components as appropriate. For example, a polymerase or other reagent may be used that exhibits a desired activity at a particular temperature or other reaction condition. In some embodiments, reagents may be used that are designed or optimized for use in hot-start amplification, for example, a polymerase may be activated after transposition or after reaching a particular temperature. Such polymerases may be antibody-based or aptamer-based. Polymerases as described herein are known in the art. Examples of such reagents may include, but are not limited to, hot-start polymerases, hot-start dNTPs, and photo-caged dNTPs. Such reagents are known and available in the art. One of skill in the art will be able to determine the optimum temperatures as appropriate for individual reagents.

Amplification of nucleic acids may be performed using specific thermal cycle machinery or equipment, and may be performed in single reactions or in bulk, such that any desired number of reactions may be performed simultaneously. In some embodiments, amplification may be performed using microfluidic or robotic devices, or may be performed using manual alteration in temperatures to achieve the desired amplification. In some embodiments, optimization may be performed to obtain the optimum reactions conditions for the particular application or materials. One of skill in the art will understand and be able to optimize reaction conditions to obtain sufficient amplification.

In certain embodiments, detection of DNA with the methods or systems of the invention requires transcription of the (amplified) DNA into RNA prior to detection.

It will be evident that detection methods of the invention can involve nucleic acid amplification and detection procedures in various combinations. The nucleic acid to be detected can be any naturally occurring or synthetic nucleic acid, including but not limited to DNA and RNA, which may be amplified by any suitable method to provide an intermediate product that can be detected. Detection of the intermediate product can be by any suitable method including but not limited to binding and activation of a CRISPR protein which produces a detectable signal moiety by direct or collateral activity.

Helicase-Dependent Amplification

In helicase-dependent amplification, a helicase enzyme is used to unwind a double stranded nucleic acid to generate templates for primer hybridization and subsequent primer-extension. This process utilizes two oligonucleotide primers, each hybridizing to the 3′-end of either the sense strand containing the target sequence or the anti-sense strand containing the reverse-complementary target sequence. The HDA reaction is a general method for helicase-dependent nucleic acid amplification.

In combining this method with a CRISPR-SHERLOCK system, the target nucleic acid may be amplified by opening R-loops of the target nucleic acid using first and second CRISPR/Cas complexes. The first and second strand of the target nucleic acid may thus be unwound using a helicase, allowing primers and polymerase to bind and extend the DNA under isothermal conditions.

The term “helicase” refers here to any enzyme capable of unwinding a double stranded nucleic acid enzymatically. For example, helicases are enzymes that are found in all organisms and in all processes that involve nucleic acid such as replication, recombination, repair, transcription, translation and RNA splicing. (Kornberg and Baker, DNA Replication, W. H. Freeman and Company (2^{nd} ed. (1992)), especially chapter 11). Any helicase that translocates along DNA or RNA in a 5′ to 3′ direction or in the opposite 3′ to 5′ direction may be used in present embodiments of the invention. This includes helicases obtained from prokaryotes, viruses, archaea, and eukaryotes or recombinant forms of naturally occurring enzymes as well as analogues or derivatives having the specified activity. Examples of naturally occurring DNA helicases, described by Kornberg and Baker in chapter 11 of their book, DNA Replication, W. H. Freeman and Company (2^{nd} ed. (1992)), include E. coli helicase I, II, III, & IV, Rep, DnaB, PriA, PcrA, T4 Gp4lhelicase, T4 Dda helicase, T7 Gp4 helicases, SV40 Large T antigen, yeast RAD. Additional helicases that may be useful in HDA include RecQ helicase (Harmon and Kowalczykowski, J. Biol. Chem. 276:232-243 (2001)), thermostable UvrD helicases from T. tengcongensis (disclosed in this invention, Example XII) and T. thermophilus (Collins and McCarthy, Extremophiles. 7:35-41. (2003)), thermostable DnaB helicase from T. aquaticus (Kaplan and Steitz, J. Biol. Chem. 274:6889-6897 (1999)), and MCM helicase from archaeal and eukaryotic organisms ((Grainge et al., Nucleic Acids Res. 31:4888-4898 (2003)).

A traditional definition of a helicase is an enzyme that catalyzes the reaction of separating/unzipping/unwinding the helical structure of nucleic acid duplexes (DNA, RNA or hybrids) into single-stranded components, using nucleoside triphosphate (NTP) hydrolysis as the energy source (such as ATP). However, it should be noted that not all helicases fit this definition anymore. A more general definition is that they are motor proteins that move along the single-stranded or double stranded nucleic acids (usually in a certain direction, 3′ to 5′ or 5 to 3, or both), i.e. translocases, that can or cannot unwind the duplexed nucleic acid encountered. In addition, some helicases simply bind and “melt” the duplexed nucleic acid structure without an apparent translocase activity.

Helicases exist in all living organisms and function in all aspects of nucleic acid metabolism. Helicases are classified based on the amino acid sequences, directionality, oligomerization state and nucleic-acid type and structure preferences. The most common classification method was developed based on the presence of certain amino acid sequences, called motifs. According to this classification helicases are divided into 6 super families: SF1, SF2, SF3, SF4, SF5 and SF6. SF1 and SF2 helicases do not form a ring structure around the nucleic acid, whereas SF3 to SF6 do. Superfamily classification is not dependent on the classical taxonomy.

DNA helicases are responsible for catalyzing the unwinding of double-stranded DNA (dsDNA) molecules to their respective single-stranded nucleic acid (ssDNA) forms. Although structural and biochemical studies have shown how various helicases can translocate on ssDNA directionally, consuming one ATP per nucleotide, the mechanism of nucleic acid unwinding and how the unwinding activity is regulated remains unclear and controversial (T. M. Lohman, E. J. Tomko, C. G. Wu, “Non-hexameric DNA helicases and translocases: mechanisms and regulation,” Nat Rev Mol Cell Biol 9:391-401 (2008)). Since helicases can potentially unwind all nucleic acids encountered, understanding how their unwinding activities are regulated can lead to harnessing helicase functions for biotechnology applications.

The term “HDA” refers to Helicase Dependent Amplification, which is an in vitro method for amplifying nucleic acids by using a helicase preparation for unwinding a double stranded nucleic acid to generate templates for primer hybridization and subsequent primer-extension. This process utilizes two oligonucleotide primers, each hybridizing to the 3′-end of either the sense strand containing the target sequence or the anti-sense strand containing the reverse-complementary target sequence. The HDA reaction is a general method for helicase-dependent nucleic acid amplification.

The invention comprises use of any suitable helicase known in the art. These include, but are not necessarily limited to, UvrD helicase, CRISPR-Cas3 helicase, E. coli helicase I, E. coli helicase II, E. coli helicase III, E. coli helicase IV, Rep helicase, DnaB helicase, PriA helicase, PcrA helicase, T4 Gp41 helicase, T4 Dda helicase, SV40 Large T antigen, yeast RAD helicase, RecD helicase, RecQ helicase, thermostable T. tengcongensis UvrD helicase, thermostable T. thermophilus UvrD helicase, thermostable T. aquaticus DnaB helicase, Dda helicase, papilloma virus E1 helicase, archaeal MCM helicase, eukaryotic MCM helicase, and T7 Gp4 helicase.

In particularly preferred embodiments, the helicase comprises a super mutation. In particular embodiments, although the E. coli mutation has been described, the mutations were generated by sequence alignment (e.g. D409A/D410A for TteUvrd) and result in thermophilic enzymes working at lower temperatures, such as 37° C., which is advantageous for amplification methods and systems described herein. In some embodiments, the super mutations is an aspartate to alanine mutation, with position based on sequence alignment. In some embodiments, the super mutant helicase is selected from WP_003870487.1 Thermoanaerobacter ethanolicus 403/404, WP_049660019.1 Bacillus sp. FJAT-27231 407/408, WP_034654680.1 Bacillus megaterium 415/416, WP_095390358.1 Bacillus simplex 407/408, and WP_055343022.1 Paeniclostridium sordellii 402/403.

An “individual discrete volume” is a discrete volume or discrete space, such as a container, receptacle, or other defined volume or space that can be defined by properties that prevent and/or inhibit migration of nucleic acids and reagents necessary to carry out the methods disclosed herein, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof. By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the used of non-walled, or semipermeable is that some reagents, such as buffers, chemical activators, or other agents maybe passed in Applicants' through the discrete volume, while other material, such as target molecules, maybe maintained in the discrete volume or space. Typically, a discrete volume will include a fluid medium, (for example, an aqueous solution, an oil, a buffer, and/or a media capable of supporting cell growth) suitable for labeling of the target molecule with the indexable nucleic acid identifier under conditions that permit labeling. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain example embodiments, the individual discrete volumes are the wells of a microplate. In certain example embodiments, the microplate is a 96 well, a 384 well, or a 1536 well microplate.

### Incubating

Methods of detection and or quantifying using the systems disclosed herein can comprise incubating the sample or set of samples under conditions sufficient to allow binding of the guide RNAs to one or more target molecules. In certain example embodiments, the incubation time of the present invention may be shortened. The assay may be performed in a period of time required for an enzymatic reaction to occur. One skilled in the art can perform biochemical reactions in 5 minutes (e.g., 5 minute ligation). Incubating may occur at one or more temperatures over timeframes between about 10 minutes and 3 hours, preferably less than 200 minutes, 150 minutes, 100 minutes, 75 minutes, 60 minutes, 45 minutes, 30 minutes, or 20 minutes, depending on sample, reagents and components of the system. In some embodiments, incubating is performed at one or more temperatures between about 20° C. and 80° C., in some embodiments, about 37° C.

### Activating

Activating of the CRISPR effector protein occurs via binding of the guide RNAs to the one or more target molecules, wherein activating the CRISPR effector protein results in modification of the detection construct such that a detectable positive signal is generated.

### Detecting a Signal

Detecting may comprise visual observance of a positive signal relative to a control. Detecting may comprise a loss of signal or presence of signal at one or more capture regions, for example colorimetric detection, or fluorescent detection. In certain example embodiments, further modifications may be introduced that further amplify the detectable positive signal. For example, activated CRISPR effector protein collateral activation may be used to generate a secondary target or additional guide sequence, or both. In one example embodiment, the reaction solution would contain a secondary target that is spiked in at high concentration. The secondary target may be distinct from the primary target (i.e. the target for which the assay is designed to detect) and in certain instances may be common across all reaction volumes. A secondary guide sequence for the secondary target may be protected, e.g. by a secondary structural feature such as a hairpin with an RNA loop, and unable to bind the second target or the CRISPR effector protein. Cleavage of the protecting group by an activated CRISPR effector protein (i.e. after activation by formation of complex with the primary target(s) in solution) and formation of a complex with free CRISPR effector protein in solution and activation from the spiked in secondary target. In certain other example embodiments, a similar concept is used with free guide sequence to a secondary target and protected secondary target. Cleavage of a protecting group off the secondary target would allow additional CRISPR effector protein, guide sequence, secondary target sequence to form. In yet another example embodiment, activation of CRISPR effector protein by the primary target(s) may be used to cleave a protected or circularized primer, which would then be released to perform an isothermal amplification reaction, such as those disclosed herein, on a template for either secondary guide sequence, secondary target, or both. Subsequent transcription of this amplified template would produce more secondary guide sequence and/or secondary target sequence, followed by additional CRISPR effector protein collateral activation.

### Quantifying

In particular methods, comparing the intensity of the one or more signals to a control is performed to quantify the nucleic acid in the sample. The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control patient (can be stored sample or previous sample measurement) with a known outcome; normal tissue, fluid, or cells isolated from a subject, such as a normal patient or the patient having a condition of interest.

The intensity of a signal is “significantly” higher or lower than the normal intensity if the signal is greater or less, respectively, than the normal or control level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternatively, the signal can be considered “significantly” higher or lower than the normal and/or control signal if the amount is at least about two, and preferably at least about 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, two times, three times, four times, five times, or more, or any range in between, such as 5%-100%, higher or lower, respectively, than the normal and/or control signal. Such significant modulation values can be applied to any metric described herein, such as altered level of expression, altered activity, changes in biomarker inhibition, changes in test agent binding, and the like.

In some embodiments, the detectable positive signal may be a loss of fluorescent signal relative to a control, as described herein. In some embodiments, the detectable positive signal may be detected on a lateral flow device, as described herein.

### Applications of Detection Methods

In certain example embodiments, the systems, devices, and methods, disclosed herein are directed to detecting the presence of one or more microbial agents in a sample, such as a biological sample obtained from a subject. In certain example embodiments, the microbe may be a bacterium, a fungus, a yeast, a protozoan, a parasite, or a virus. Accordingly, the methods disclosed herein can be adapted for use in other methods (or in combination) with other methods that require quick identification of microbe species, monitoring the presence of microbial proteins (antigens), antibodies, antibody genes, detection of certain phenotypes (e.g. bacterial resistance), monitoring of disease progression and/or outbreak, and antibiotic screening. Because of the rapid and sensitive diagnostic capabilities of the embodiments disclosed here, detection of microbe species type, down to a single nucleotide difference, and the ability to be deployed as a POC device, the embodiments disclosed herein may be used as guide therapeutic regimens, such as a selection of the appropriate antibiotic or antiviral. The embodiments disclosed herein may also be used to screen environmental samples (air, water, surfaces, food etc.) for the presence of microbial contamination.

Disclosed is a method to identify microbial species, such as bacterial, viral, fungal, yeast, or parasitic species, or the like. Particular embodiments disclosed herein describe methods and systems that will identify and distinguish microbial species within a single sample, or across multiple samples, allowing for recognition of many different microbes. The present methods allow the detection of pathogens and distinguishing between two or more species of one or more organisms, e.g., bacteria, viruses, yeast, protozoa, and fungi or a combination thereof, in a biological or environmental sample, by detecting the presence of a target nucleic acid sequence in the sample. A positive signal obtained from the sample indicates the presence of the microbe. Multiple microbes can be identified simultaneously using the methods and systems of the invention, by employing the use of more than one effector protein, wherein each effector protein targets a specific microbial target sequence. In this way, a multi-level analysis can be performed for a particular subject in which any number of microbes can be detected at once. In some embodiments, simultaneous detection of multiple microbes may be performed using a set of probes that can identify one or more microbial species.

The systems and methods of detection can be used to identify single nucleotide variants, detection based on rRNA sequences, screening for drug resistance, monitoring microbe outbreaks, genetic perturbations, and screening of environmental samples, as described in International Patent Application No. PCT/US2018/054472 filed Oct. 22, 2018 at [0183]-[0327], incorporated herein by reference.

In certain example embodiments, the systems, devices, and methods disclosed herein may be used for biomarker detection. For example, the systems, devices and method disclosed herein may be used for SNP detection and/or genotyping. The systems, devices and methods disclosed herein may be also used for the detection of any disease state or disorder characterized by aberrant gene expression. Aberrant gene expression includes aberration in the gene expressed, location of expression and level of expression. Multiple transcripts or protein markers related to cardiovascular, immune disorders, and cancer among other diseases may be detected. In certain example embodiments, the embodiments disclosed herein may be used for cell free DNA detection of diseases that involve lysis, such as liver fibrosis and restrictive/obstructive lung disease. In certain example embodiments, the embodiments could be utilized for faster and more portable detection for pre-natal testing of cell-free DNA. The embodiments disclosed herein may be used for screening panels of different SNPs associated with, among others, cardiovascular health, lipid/metabolic signatures, ethnicity identification, paternity matching, human ID (e.g. matching suspect to a criminal database of SNP signatures). The embodiments disclosed herein may also be used for cell free DNA detection of mutations related to and released from cancer tumors. The embodiments disclosed herein may also be used for detection of meat quality, for example, by providing rapid detection of different animal sources in a given meat product. Embodiments disclosed herein may also be used for the detection of GMOs or gene editing related to DNA. As described herein elsewhere, closely related genotypes/alleles or biomarkers (e.g. having only a single nucleotide difference in a given target sequence) may be distinguished by introduction of a synthetic mismatch in the gRNA.

In an aspect, the invention relates to a method for detecting target nucleic acids in samples, comprising:

distributing a sample or set of samples into one or more individual discrete volumes, the individual discrete volumes comprising a CRISPR system according to the invention as described herein;

incubating the sample or set of samples under conditions sufficient to allow binding of the one or more guide RNAs to one or more target molecules;

activating the CRISPR effector protein via binding of the one or more guide RNAs to the one or more target molecules, wherein activating the CRISPR effector protein results in modification of the RNA-based masking construct such that a detectable positive signal is generated; and

detecting the detectable positive signal, wherein detection of the detectable positive signal indicates a presence of one or more target molecules in the sample.

The sensitivity of the assays described herein are well suited for detection of target nucleic acids in a wide variety of biological sample types, including sample types in which the target nucleic acid is dilute or for which sample material is limited. Biomarker screening may be carried out on a number of sample types including, but not limited to, saliva, urine, blood, feces, sputum, and cerebrospinal fluid. In certain embodiments, the sample is from bone marrow or peripheral blood. The embodiments disclosed herein may also be used to detect up- and/or down-regulation of genes. For example, a sample may be serially diluted such that only over-expressed genes remain above the detection limit threshold of the assay.

In certain embodiments, the present invention provides steps of obtaining a sample of biological fluid (e.g., urine, blood plasma or serum, sputum, cerebral spinal fluid), and extracting the DNA. The mutant nucleotide sequence to be detected, may be a fraction of a larger molecule or can be present initially as a discrete molecule.

In certain embodiments, DNA is isolated from plasma/serum of a cancer patient. For comparison, DNA samples isolated from neoplastic tissue and a second sample may be isolated from non-neoplastic tissue from the same patient (control), for example, lymphocytes. The non-neoplastic tissue can be of the same type as the neoplastic tissue or from a different organ source. In certain embodiments, blood samples are collected and plasma immediately separated from the blood cells by centrifugation. Serum may be filtered and stored frozen until DNA extraction.

In embodiments, the sample can be a cryopreserved or a fresh sample. Steps of pelleting and extracting can be performed on cryopreserved or fresh samples. In embodiments, cells from a sample are washed and pelleted. In embodiments, lysis buffer can be used, with or without PBS washes prior to pelleting, in particular embodiments, the sample treated with lysis buffer is a fresh sample. Extraction of RNA can be performed in pelleted samples using commercially available kits, for example, the Qiagen RNeasy Kit.

In certain example embodiments, target nucleic acids are detected directly from a crude or unprocessed sample, such as blood, serum, saliva, cerebrospinal fluid, sputum, or urine. In certain example embodiments, the target nucleic acid is cell free DNA.

### EXAMPLES

### Example 1—Guide Design

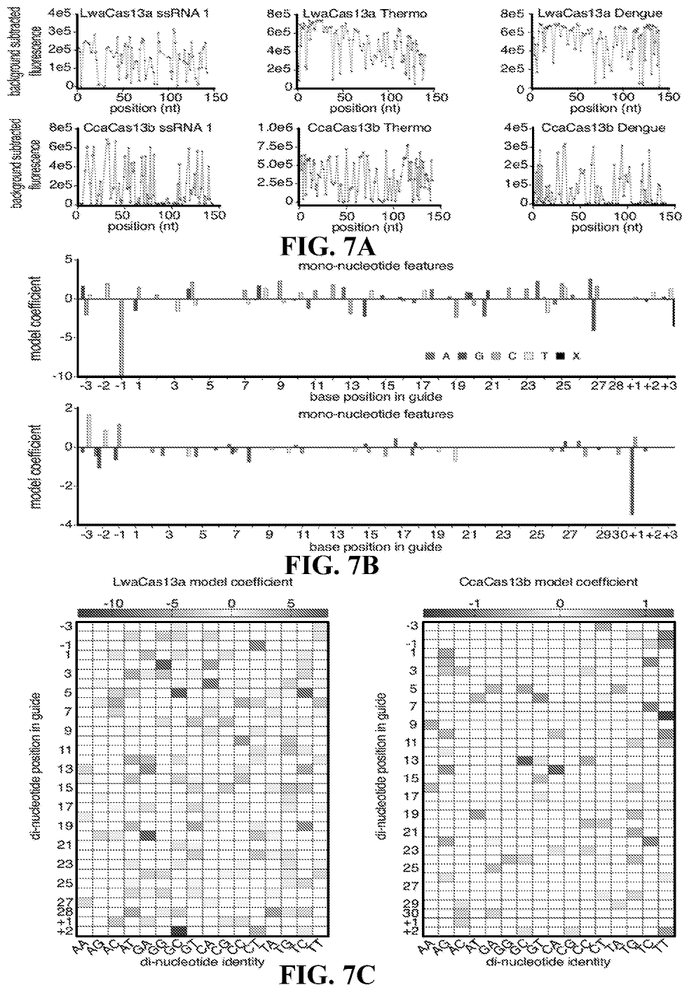

Previous tiling of SHERLOCK guides along targets has demonstrated significant variation in collateral activity between guide RNAs with LwaCas13a and CcaCas13b¹, which has an effect on the overall kinetics and sensitivity of the assay. Although a sequence constraint known as a protospacer flanking site (PFS) exists for Cas13 targeting^{3,6}, many guide RNAs without the correct PFS retain activity. Applicants therefore hypothesized that some combination of the PFS and other sequence and guide features might be driving the efficacy of Cas13 detection. Applicants applied a machine learning approach to train a logistic regression model on the collateral activity of hundreds of guides, using a combination of guide sequence, flanking target sequence, guide position, and guide GC content as input features (FIG. 1a). Applicants designed a panel of 410 crRNAs for LwaCas13a and 476 crRNAs for CcaCas13b across five different ssRNA targets: Ebola, Zika, the thermonuclease transcript from S. aureus, Dengue, and a synthetic ssRNA target (ssRNA 1). Using in vitro transcription to express these guides, Applicants evaluated the resulting collateral activity of LwaCas13a and CcaCas13b by fluorescent reporter assays and found significant variation between the crRNAs (FIG. 1b and FIG. 7a).

Given the wide variance of guide efficiencies for both LwaCas13a and CcaCas13b, Applicants designed a machine learning model that would select for the “best” performing guides for each enzyme as follows. As a majority of LwaCas13a guides had activity above background (FIG. 1b, FIG. 7a), Applicants selected, on a per-target basis, guides with 2-fold activity over the median activity as “best” performing guides. By contrast, a majority of CcaCas13b guides were near background, (FIG. 1b, FIG. 7a), so “best” performing guides were classified as the top quintile for each target tested. For each ortholog, a logistic regression model was trained to distinguish best performing guides from all other guides, based on the input features. The length of the flanking target region was considered as a free parameter and selected during cross-validation by maximizing the area under the curve (AUC) of the receiver operator characteristic (ROC) for each machine learning model. The data was split into train/test/validation sets and the training and test sets were used for training the logistic model with three-fold cross validation and a hyperparameter search. This training process resulted in models with AUC of 0.84 and 0.89 for LwaCas13a and CcaCas13b, respectively (FIG. 1c). Examination of the full feature set for the machine learning model (FIG. 7b, 7c) revealed strong weights for both orthologs in the guide sequence and flanking regions. One of the key features that stood out were weights that recapitulated the known PFS preferences of the enzymes (3′ H for LwaCas13a and 5′-D/3 NAA for CcaCas3b) (FIG. 1d)^{3,6}, providing biological validation to the machine learning model. To make the design tool easily accessible and usable by the community, Applicants provide a simple web tool (sherlock.genome-engineering.org) for LwaCas13a and CcaCas13b guide design.

### Example 2—Guide Validation

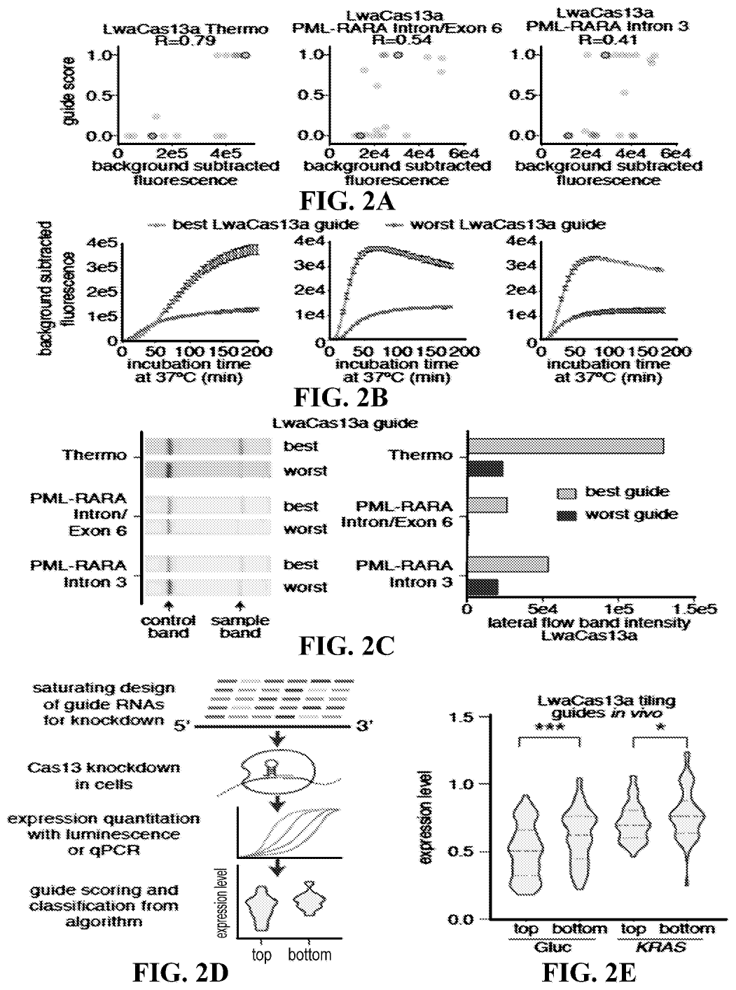

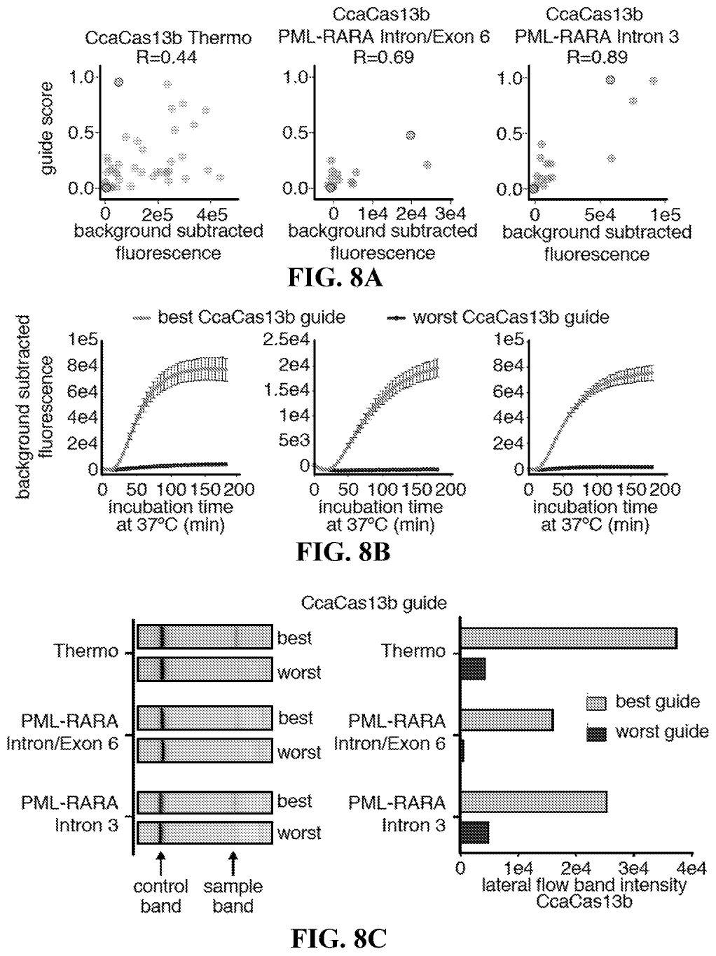

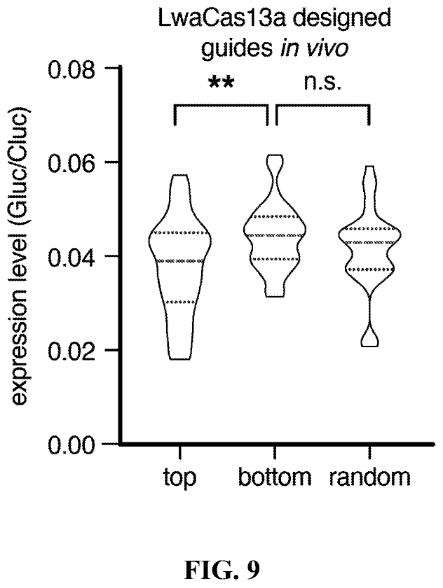

To further validate the machine learning models beyond the cross-validation, Applicants designed a panel of new crRNAs using the machine learning model targeting either the thermonuclease transcript or two additional transcripts from the long and short isoforms of the PML/RARA fusion associated with acute promyelocytic leukemia (APML). Applicants found that both the LwaCas13a and CcaCas13b models succeeded at predicting guide RNA activity (LwaCas13a model validation has R values of 0.79, 0.54, and 0.41; CcaCas13b model validation has R values of 0.44, 0.69, and 0.89) (FIG. 2a, FIG. 8a). Additionally, the best and worst predicted crRNAs display drastically different kinetics and sensitivity (FIG. 2b, FIG. 8b). Although the improvement in kinetics for best predicted crRNAs is relevant for increasing the speed of all SHERLOCK assays, the signal increase is especially relevant for portable versions of the test, as color generation on the lateral flow strips is sensitive to the overall collateral activity levels. While the guide model was trained for maximizing overall signal generation, the increase in kinetics was an added benefit that was not explicitly trained for in the machine learning model development. Applicants evaluated the best and worst predicted crRNAs for the thermonuclease, short APML, and long APML targets on lateral flow strips and found that only the best predicted crRNAs generated a functional test suitable for portable detection (FIG. 2c, FIG. 8c). Moreover, Applicants also validated the LwaCas13a prediction model for in vivo transcript knockdown by targeting the Gaussia luciferase (Gluc) transcript in HEK293FT cells and evaluating previously published LwaCas13a mammalian RNA knockdown data of reporter and endogenous transcripts (FIG. 2d)¹⁴. Applicants found that guides predicted to have strong activity were significantly more effective at knockdown of Gluc and KRAS (FIG. 2e) and that Gluc guides with predicted good performance outperformed guides either with poor predicted performance or selected randomly (FIG. 9).

### Example 3—One-Pot Assay

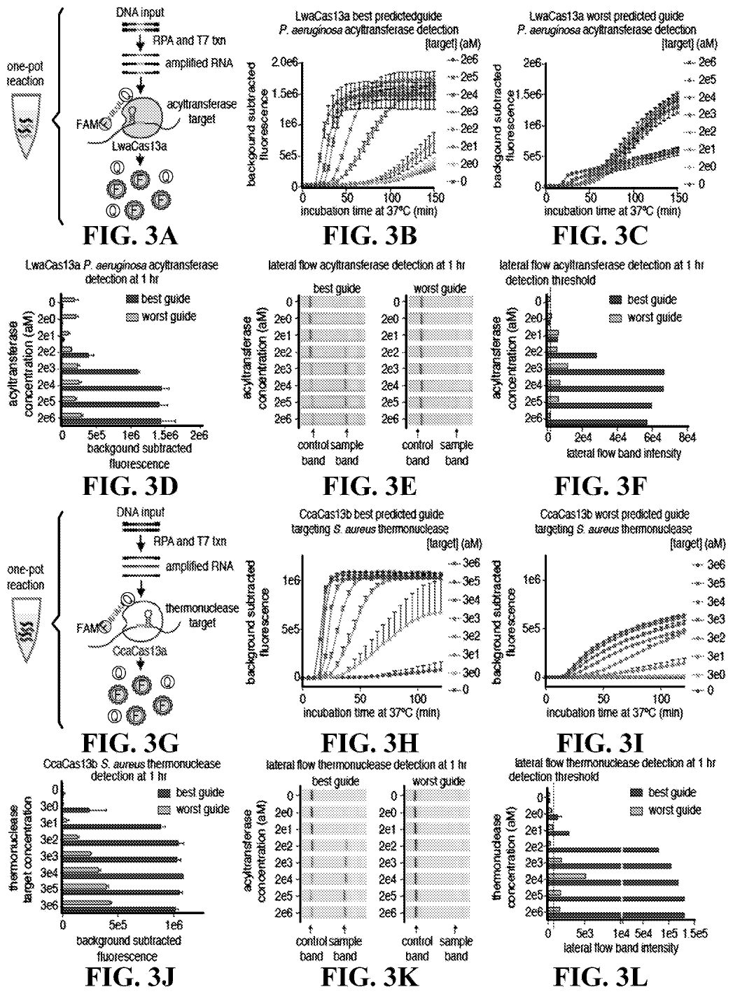

Previous versions of the SHERLOCK assay have been a two-step format with an initial recombinase polymerase amplification (RPA)¹⁹ followed by T7 transcription and Cas13 detection. To simplify the SHERLOCK assay, Applicants focused on optimizing a one-pot amplification and detection protocol by combining both steps into a single reaction with the best predicted crRNAs. Applicants designed a one-pot SHERLOCK assay for a synthetic acyltransferase transcript derived from Pseudomonas aeruginosa, a significant human pathogen that requires rapid diagnosis. Applicants found that the best predicted crRNA for LwaCas13a allowed for fast and highly-sensitive (20 aM) detection of acyltransferase in a one-pot reaction format compared to the worst predicted crRNA (FIG. 3a-d). Additionally, the best predicted crRNA enabled an acyltransferase lateral flow assay with sensitivity down to 20 aM (FIG. 3e, 3f). Similarly, for CcaCas13b, Applicants used the guide prediction machine learning model to generate a one-pot SHERLOCK assay for detection of the thermonuclease transcript (FIG. 3g). As with LwaCas13a, Applicants found that CcaCas13b could achieve fast and sensitive detection down to 3 aM by fluorescence (FIG. 3h-j) and 20 aM by portable lateral flow (FIG. 3k, 3l). The optimized one-pot format was readily extendable to additional targets, including the Ea175 and Ea81 transcripts from Treponema denticola, a gram-negative bacteria that can cause severe periodontal disease, and could be adapted for sensitive lateral flow tests (FIG. 10A-10F).

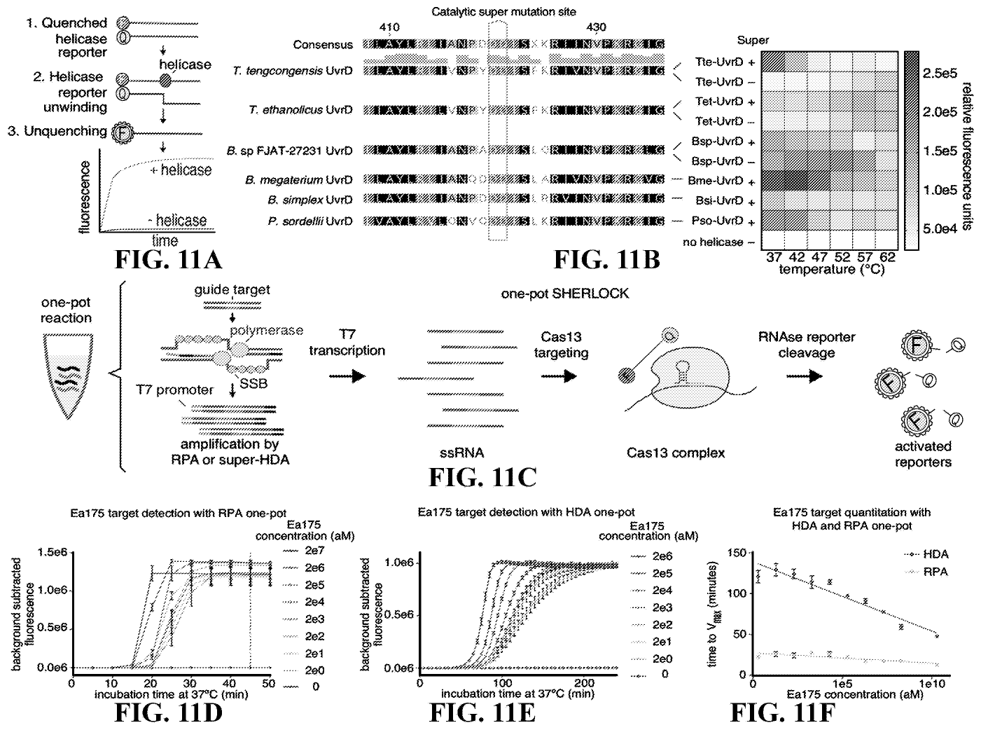

To achieve even higher sensitivity with one-pot assays, Applicants explored alternative amplification strategies, which could provide less bias and result in a more quantitative assay. Helicase displacement amplification (HDA)²⁰ relies on helicases to separate the DNA duplex and allow for primer invasion and amplification, usually at high temperatures like 65° C. To enable rapid HDA, Applicants profiled a set of UvrD helicase orthologs with engineered mutations²¹ with a helicase reporter assay (FIG. 11a, 11b)²² and found several candidates with strong helicase activity at 37° C., including Super UvrD from Thermoanaerobacter tengcongensis (TteUvrD), which allowed for 37° C. isothermal amplification and compatibility with Cas13-based collateral detection. Applicants combined Super TteUvrD with polymerases, single-stranded binding proteins, and LwaCas13a to create a one-pot super HDA SHERLOCK reaction, which was capable of single molecule detection of the Ea175 target at 100 minutes and was highly quantitative (FIG. 11c-11e).

### Example 4—Multiplexing

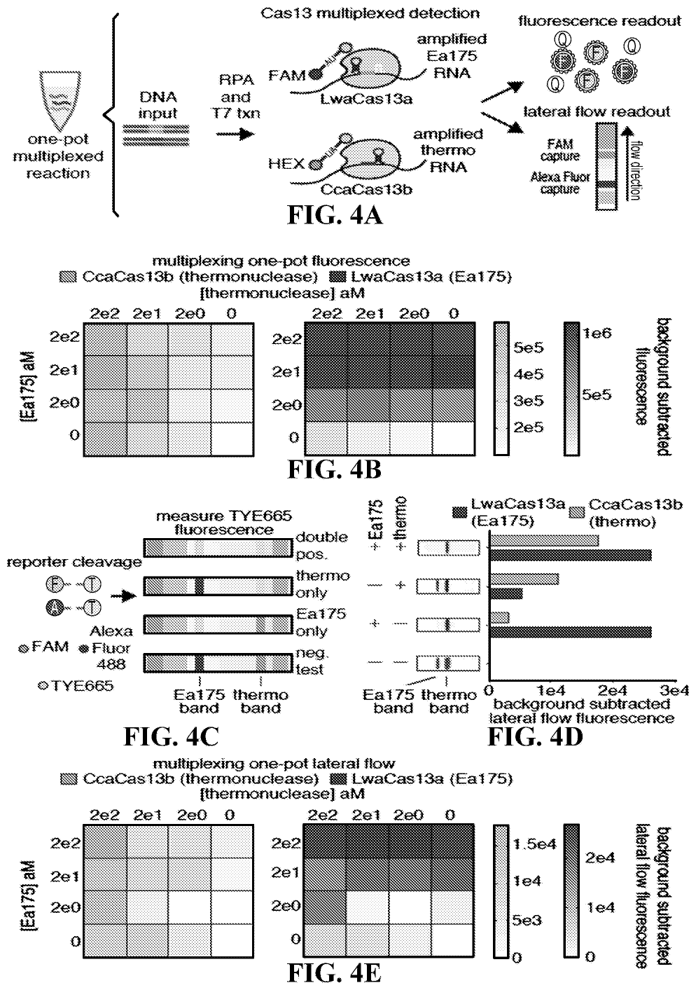

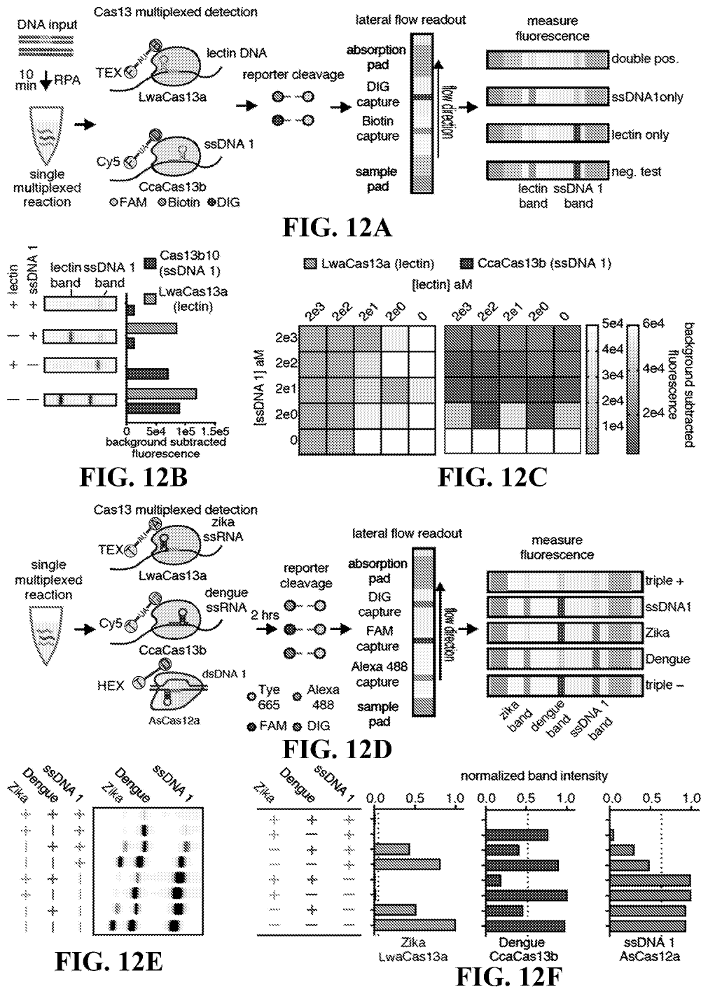

Applicants further expanded the one-pot RPA SHERLOCK assay to allow for multiplexing of multiple targets (FIG. 4a). Applicants first tested whether one-pot SHERLOCK could simultaneously detect two targets, Ea175 and thermonuclease, using LwaCas13a and CcaCas13b, respectively. By detecting the collateral activity of each enzyme in separate fluorescent channels, FAM and HEX, Applicants were able to achieve 2 aM detection of each target (FIG. 4b). Next, Applicants adapted the lateral flow format to allow for detection of two targets. As the previous lateral flow design relied on general capture of antibody that was not bound by intact reporter RNAs¹, it is not suitable for detecting two targets. Instead, Applicants adapted a lateral flow approach with two separate detection lines consisting of either deposited streptavidin or anti-DIG antibodies. These lines capture reporter RNA decorated with a fluorophore and either Biotin or DIG, allowing fluorescent visualization of signal loss at detection lines due to collateral activity and cleavage of corresponding reporter RNA. Applicants evaluated this lateral flow design using a two-step SHERLOCK format for detection of lectin DNA and a synthetic DNA target (ssDNA 1) (FIG. 12a), and found that Applicants could detect down to 2 aM of each target (FIG. 12b, 12c). Applicants then applied the one-pot multiplexed SHERLOCK assay for thermonuclease and Ea175 to the new lateral flow format (FIG. 4c) and found that Applicants could detect down to 20 aM of each target successfully (FIG. 4d,e). As this lateral flow design can be extended further by depositing any molecule that is part of an orthogonal hybridization pair, Applicants developed lateral flow strips capable of detecting three targets simultaneously by striping the anti-Alexa 488 antibody to capture Alexa 488 on a reporter DNA (FIG. 12d). By augmenting the lateral flow assay with Cas12a from Acidaminococcus sp. BV3L6 (AsCas12a), Applicants were able to independently assay a third target in an additional cleavage channel sensing DNA collateral activity¹. This design was capable of independently assaying three targets, Zika ssRNA, Dengue ssRNA, and ssDNA1 simultaneously (FIG. 12e, 12f).

### Example 5—Optimized Clinical Detection

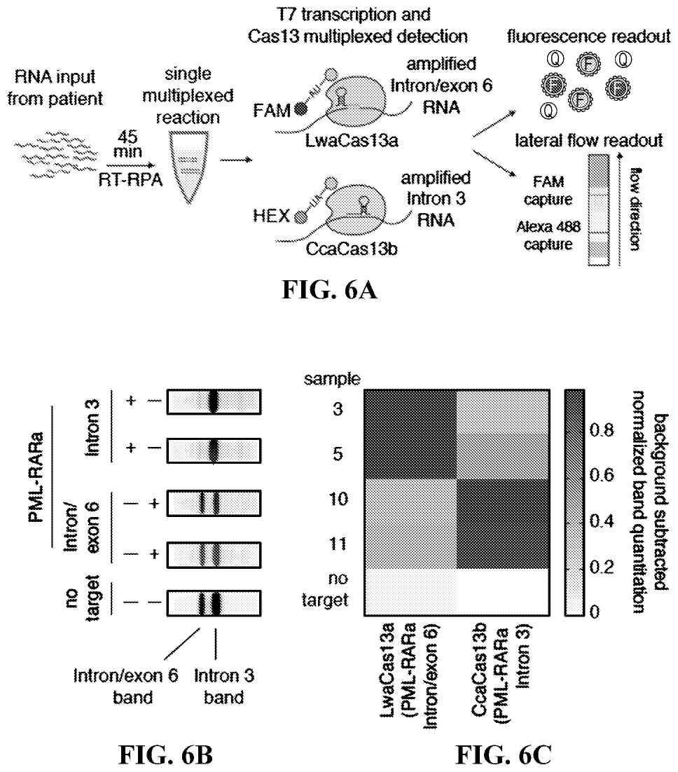

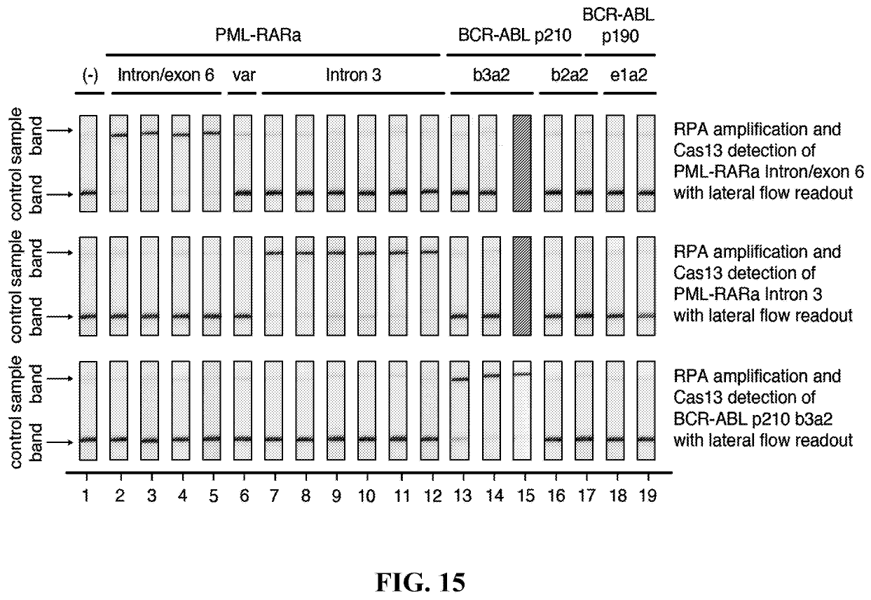

Lastly, Applicants sought to apply SHERLOCK detection to a clinical setting, where using the best crRNA for a given target is essential for fast and sensitive performance. Acute promyelocytic leukemia (APML) and acute lymphocytic leukemia (ALL) cancers are caused by chromosomal fusions in the transcribed mRNA, and distinguishing these rapidly is critical for effective treatment and prognosis²³. To design robust clinical-grade SHERLOCK assays, Applicants employed the Cas13 guide design tool to predict top guides for three fusion transcripts characteristic of APML and ALL cancers: PML-RARa Intron/exon 6 fusion, PML-RARa Intron 3 fusion, and BCR-ABL p210 b3a2 fusion²³ (FIG. 5a). The developed SHERLOCK assay for these three targets (FIGS. 13A-13D) was used to predict APML or ALL presence across a blinded set of 17 patient bone marrow samples, as well as 2 known samples (samples 12 and 15 in FIG. 5). Cas13 detection using the best predicted guide achieved clear fluorescence detection in 45 minutes or less for all samples verified by RT-PCR (FIG. 5b,c,d, FIG. 14A-14E). Detection with a lateral flow readout also yielded clear identification of the RNA fusion present in every sample (FIG. 5e, FIG. 15). Lastly, Applicants showed that the multiplexed lateral flow test could be deployed to simultaneously test for multiple fusion transcripts (FIG. 6), enabling a simple, rapid, and portable test that can detect several cancer fusion transcripts simultaneously.

### Discussion

The SHERLOCK platform is a low-cost CRISPR-based diagnostic that enables single-molecule detection of DNA or RNA with single-nucleotide specificity^{1,2,10}. Nucleic acid detection with SHERLOCK relies on the collateral activity of Cas13 and Cas12 to promiscuously cleave reporters upon target recognition^{3,4,7}. SHERLOCK is capable of single-molecule detection in less than an hour and can be used for multiplexed target detection when using CRISPR enzymes with orthogonal cleavage preference, such as Cas13a from Leptotrichia wadei (LwaCas13a), Cas13b from Capnocytophaga canimorsus Cc5 (CcaCas13b), and Cas12a from Acidaminococcus sp. BV3L6 (AsCas12a)^{1,2,9-12}. These enzymes have also been used for other applications both in vivo and in vitro^{1,2,9-16}, and predictive Cas13 guide design tools would be broadly beneficial in the design of Cas13-based experiments or assays, such as knockdown of transcripts by Cas13 in mammalian cells. In particular, an accurate model for activity-based Cas13 guide selection would facilitate design of optimal SHERLOCK assays, especially in applications requiring high-activity guides like lateral flow detection.

Previously, software for the design and prediction of Cas9 guides for activity and off-target minimization have been developed using machine learning approaches 17,18, broadening the use the use of the technology. Applicants therefore worked to develop a similar tool for Cas13 by using a machine learning approach.

Together, these results herein demonstrate that SHERLOCK assays can be reliably designed with high sensitivity and fast kinetics using a machine learning approach, accessible at sherlock.genome-engineering.org. This guide design tool has broad applicability for both in vitro and in vivo RNA targeting applications and can be readily extended to include other useful Cas13 and Cas12 orthologs with collateral activity, including Cas13d^{13,24}, Cas12a^{8,9,11}, Cas12b^{5,12}, and many other Cas12/Cas13 family members^{7,25}. Using the design tool, Applicants generated highly sensitive assays suitable for portable lateral flow detection of one or two targets using LwaCas13a and CcaCas13b, which can be performed in a single step, reducing pipetting steps and eliminating potential contamination of post-amplification samples. Additionally, by utilizing DNA collateral detection with AsCas12a, Applicants can perform multiplexing of three targets in a lateral flow format. With these improvements, SHERLOCK can now achieve multiplexing of up to four targets simultaneously by fluorescence¹ and three targets by lateral flow. Applicants also apply helicase engineering to develop a new CRISPR-detection compatible amplification method, super HDA, and demonstrate the quantitative nature of super HDA SHERLOCK. Finally, Applicants demonstrate the facile applicability of the guide design model to develop a clinically relevant test for APML and ALL cancers with high sensitivity and performance in a portable lateral flow format. The advances here increase the accessibility of the SHERLOCK platform, deploying it as a simple, portable nucleic acid diagnostic with broad clinical utility and provide a user-friendly web tool for Cas13 guide design for both in vivo RNA targeting and SHERLOCK assays.

### Methods

### Protein Expression and Purification of Cas13

Expression and purification of LwaCas13a and CcaCas13b was performed as previously described^{1,2}. In brief, Applicants transformed bacterial expression vectors into Rosetta™ 2(DE3)pLysS Singles Competent Cells (Millipore) and scaled up bacterial growth in 4 L of Terrific Broth 4 growth media (TB). Cell pellets were lysed by high-pressure cell disruption using the LM20 Microfluidizer system at 27,000 PSI and freed protein was bound via StrepTactin Sepharose (GE) resin. After washing, protein was released from the resin via SUMO protease digestion overnight and protein was subsequently purified by cation exchange chromatography and then gel filtration purification using an AKTA PURE FPLC (GE Healthcare Life Sciences). Eluted protein was then concentrated into Storage Buffer (600 mM NaCl, 50 mM Tris-HCl pH 7.5, 5% glycerol, 2 mM DTT) and frozen at −80° C. for storage.

Nucleic Acid Target and crRNA Preparation

Nucleic acid targets and crRNAs were prepared as previously described^{1,2}. Briefly, targets were either used as ssDNA or PCR amplified with NEBNext PCR master mix, gel extracted, and purified using MinElute gel extraction kits (Qiagen). For RNA detection reactions, RNA was prepared by using either ssDNA targets with double-stranded T7-promoter regions or fully double-stranded PCR products in T7 RNA synthesis reactions at 30° C. using the HiScribe T7 Quick High Yield RNA Synthesis Kit (New England Biolabs). RNA was then purified using MEGAclear Transcription Clean-up kit (Thermo Fisher).

crRNAs were synthesized by using ultramer ssDNA substrates (IDT) that were double stranded in the T7 promoter region through an annealed primer. Synthesized crRNAs were prepared using these templates in T7 expression assays at 37C using the HiScribe T7 Quick High Yield RNA Synthesis kit (NEB). RNAs were then purified using RNAXP clean beads (Beckman Coulter) at 2× ratio of beads to reaction volume, with an additional 1.8× supplementation of isopropanol (Sigma).

All crRNA and target sequences are listed in Tables 1 and 2, respectively.

### Fluorescent Cleavage Assay

Cas13 detection assays were performed as previously described ^{1,2}. In brief, 45 nM Cas13 protein (either CcaCas13b or LwaCas13a), 20 nM crRNA, 1 nM target RNA, 125 nM RNAse Alert v2 (Invitrogen), and 1 unit/μL murine RNase inhibitor (NEB) were combined together in 20 μL of cleavage buffer (20 mM HEPES, 60 mM NaCl, 6 mM MgCl2, pH 6.8). Reactions were incubated at 37° C. on a Biotek plate reader for 3 hours with fluorescent kinetic measurements taken every 5 minutes.

SHERLOCK Nucleic Acid Detection with RPA

For RPA reactions, primers were designed using NCBI Primer-BLAST²⁶ under default parameters except for (100-140 nt), primer melting temperatures (54° C.-67° C.), and primer size (30-35 nt). All primers were ordered as DNA (Integrated DNA Technologies).

One-pot SHERLOCK-RPA reactions were carried out as previously described^{1,2} with slight modifications. Reactions were prepared with the following reagents (added in order): 0.5× RPA rehydration and 0.5× resuspended RPA lyophilized pellet, 2 mM rNTPs, 1.1 units/μL RNAse inhibitor, 1 unit/μL T7 RNA polymerase (Lucigen), 0.96 μM total RPA primers (0.48 μM each of forward primer with T7 handle and reverse primer), 57.8 nM Cas13 protein (CcaCas13b or LwaCas13a), 23.3 nM crRNA, 136.5 nM fluorescent substrate reporter, 5 mM MgCl2, 14 mM MgAc, and varying amounts of DNA target input.

For detection with fluorescent readout, either a quenched polyU FAM reporter (TriLink) or RNAse Alert v2 (Invitrogen), were used as reporters. 20 μL reactions were incubated for 2-6 hours at 37° C. on a Biotek plate reader with kinetic measurements taken either every 2.5 or 5 minutes. All reporter sequences are listed in Table 5.

One-pot SHERLOCK-RPA reactions were modified for multiplexing by maintaining total primer concentration at 0.96 μM over all four input primers (0.24 μM each of both forward primers with T7 handle and reverse primers), maintaining crRNA concentrations at 23.3 nM (with 11.7 nM each crRNA), maintaining Cas13 total protein concentration at 57.8 nM, (28.9 nM CcaCas13b and 28.9 nM LwaCas13a), and doubling total reporter concentration (136.5 nM LwaCas13a AU-FAM reporter; 136.5 nM CcaCas13b UA-HEX reporter; see Table 5 for all reporters). 20 μL reactions were incubated for 2-6 hours at 37° C. on a Biotek plate reader with kinetic measurements in wavelengths for HEX and FAM taken every 2.5 or 5 minutes.

### Protein Expression and Purification of UvrD Helicases

UvrD Helicases sequences were ordered as E. coli codon optimized gBlocks Gene Fragments (IDT) and cloned into TwinStrep-SUMO-expression plasmid via Gibson assembly. Alanine Super-helicase' mutants were generated using PIPE-site-directed mutagenesis cloning from the TwinStrep-SUMO-UvrD Helicase expression plasmids. In brief, primers with short overlapping sequences at their ends were designed to harbor the desired changes. After incomplete-extension PCR amplification (KAPA HiFi HotStart 2× PCR), reactions were treated with Dpnl restriction endonuclease for 30 minutes at 37° C. to degrade parental plasmid. Two microliters of the reaction were directly transformed into Stble3 chemically competent E. coli cells. For expression, sequence verified plasmids were transformed into BL21(DE3)pLysE E. coli cells. For each UvrD Helicase variant, 2 L of Terrific Broth media (12 g/L tryptone, 24 g/L yeast extract, 9.4 g/L K2HPO, 2.2 g/L KH2PO4), supplemented with 100 μg/mL ampicillin, was inoculated with 20 mL of overnight starter culture and grown until OD600 0.4-0.6. Protein expression was induced with the addition of 0.5 mM IPTG and carried out for 16 hours at 21° C. with 250 RPM shaking speed. Cells were collected by centrifugation at 5,000 RPM for 10 minutes, and paste was directly used for protein purification (10-20 g total cell paste). For lysis, 10 g of bacterial paste was resuspended via stirring at 4° C. in 50 mL of lysis buffer (50 mM Tris-HCl pH 8, 500 mM NaCl, 1 mM BME (Beta-Mercapotethanol, Sigma) supplemented with 50 mg Lysozyme, 10 tablets of protease inhibitors (cOmplete, EDTA-free, Roche Diagnostics Corporation), and 500 U of Benzonase (Sigma). The suspension was passed through a LM20 microfluidizer at 25,000 psi, and lysate was cleared by centrifugation at 10,000 RPM, 4° C. for 1 hour. Lysate was incubated with 2 mL of StrepTactin superflow resin (Qiagen) for 2 hours at 4° C. on a rotary shaker. Resin bound with protein was washed three times with 10 mL of lysis buffer, followed by addition of 50 μL SUMO protease (in house) in 20 mL of IGEPAL lysis buffer (0.2% IGEPAL). Cleavage of the SUMO tag and release of native protein was carried out overnight at 4° C. in Econo-column chromatography column under gentle mixing on a table shaker. Cleaved protein was collected as flow-through, washed three times with 5 mL of lysis buffer, and checked on a SDS-PAGE gel.

Protein was diluted ion exchange buffer A containing no salt (50 mM Tris-HCl pH 8, 6 mM BME (Beta-Mercapotethanol, Sigma), 5% Glycerol, 0.1 mM EDTA) to get the starting NaCl concentration of 50 mM. Protein was then loaded onto a 5 mL Heparin HP column (GE Healthcare Life Sciences) and eluted over a NaCl gradient from 50 mM to 1 M. Fractions of eluted protein were analyzed by SDS-PAGE gel and Coomassie staining, pooled and concentrated to 1 mL using 10 MWCO centrifugal filters (Amicon). Concentrated protein was loaded in 0.5-3 mL 10 MWCO Slide-A-Lyzer Dialysis cassettes and dialyzed overnight at 4° C. against protein storage buffer (20 mM Tris-HCl, pH 7.5, 200 mM NaCl, 1 mM EDTA, 1 mM TCEP, 50% glycerol). Protein was quantified using Pierce reagent (Thermo) and stored at −20° C.

### Lateral Flow Readout of Cas13 and SHERLOCK

For single-plex detection with lateral flow readout, a FAM-RNA-biotin reporter was substituted in Cas13 or SHERLOCK reactions for the fluorescent reporter at a final concentration of 1 μM (unless otherwise indicated). 20 μL reactions were incubated between 30 and 180 minutes, after which the entire reaction was resuspended in 100 μL of HybriDetect 1 assay buffer (Milenia). Visual readout was achieved with HybriDetect 1 lateral flow strips (Milenia), and strips were imaged in a light box with a α7 III with 35-mm full-frame image sensor camera (Sony) equipped with a FE2.8/90 Macro G OSS lens.

Two-pot SHERLOCK-RPA multiplexed lateral flow reactions were adapted from previously described multiplexed fluorescent reactions^{1,2}. In brief, RPA reactions were performed with the TwistAmp® Basic (TwistDx) protocol with the exception that 280 mM MgAc was added prior to input DNA. Reactions were run with 1 μL of input for 1 hr at 37° C. Cas13 detection assays were performed with 45 nM purified Cas13, 22.5 nM crRNA, lateral flow RNA reporter (4 μM LwaCas13a multiplexed reporter; 2 μM CcaCas13b multiplexed reporter; see Table 5 for all reporters), 0.5 μL murine RNase inhibitor (New England Biolabs), and 1 μL of post-RPA input nucleic acid target in nuclease assay buffer (20 mM HEPES, 60 mM NaCl, 6 mM MgCl₂, pH 6.8). 20 μL reactions were suspended in 100 μL of HybriDetect 1 assay buffer (Milenia) and run on custom multiplexed strips (DCN Diagnostics). The custom lateral flow strips were designed to have capture lines containing Anti-digoxigenin antibodies (ab64509, abcam), Streptavidin, Anti-FITC antibodies (ab19224, abcam), and Anti-Alexa 488 antibodies (A619224, Life Technologies). The strips consisted of a 25 mm CN95 Sartorius nitrocellulose membrane, an 18 mm 6614 Ahlstrom synthetic conjugate pad for sample application, and a 22 mm Ahlstrom grade 319 paper wick pad. Strips were imaged using an Azure c400 imaging system in the Cy5 channel.

One-pot multiplexed SHERLOCK-RPA was adapted for lateral flow by lowering the CcaCas13b multiplexed reporter concentration to a concentration of 78 nM and the LwaCas13a reporter concentration to 1 μM (see Table 5 for all reporters). This was to accommodate for different fluorescent intensities observed for the reporter when binding to the DCN strips. Lateral flow reactions were resuspended in buffer, run on DCN strips, and imaged as described above.

### Fluorescent Helicase Activity Assay

Helicase substrate was generated by annealing 300 pmol of fluorescent 5′-FAM-top strand with 900 pmol of quencher 3′-BHQ1 bottom strand in lx duplex buffer (30 mM HEPES, pH 7.5; 100 mM potassium acetate) for 5 minutes at 95° C., followed by slow cool down to 4° C. (1° C./5 seconds) in PCR thermocycler. After annealing, reactions were diluted 1:10 in Nuclease free water (Gibco). Helicase unwinding assays were carried out in 20 μL reactions containing 1× Thermopol buffer (NEB), 250 nM of annealed quenched helicase substrate, 3 mM ATP or 3 mM dATP (The-UvrD dATP), 200 nM UvrD Helicase and 500 nM of capture strand oligonucleotide. To determine temperature activity profiles, reactions and no helicase control were incubated at temperatures ranging from 37° C. to 62° C. with 5° C. intervals for 60 minutes in a PCR thermocycler. Reactions were immediately transferred to a 384-well plate (Corning®) and analyzed on a fluorescent plate reader (BioTek) equipped with a FAM/HEX filter set.

SHERLOCK Nucleic Acid Detection with HDA

For detection with SHERLOCK-HDA, procedures for amplification were inspired by previously described isothermal helicase dependent amplification^{20,27} with significant modifications. Reactions were prepared with the following reagents: 1× Sau polymerase buffer (Intact Genomics), 2.5% PEG 30%, 1 mM rNTPs, 0.4 mM dNTPs, and 3 mM ATP, 1 units/μL, RNAse inhibitor, 1.5 unit/μL T7 RNA polymerase (Lucigen), 0.4 μM total HDA primers (0.2 μM each of forward primer with T7 handle and reverse primer), 43.3 nM Cas13 protein (CcaCas13b or LwaCas13a), 19.8 nM crRNA, 125 nM fluorescent substrate reporter (quenched polyU FAM reporter, TriLink), 0.2 units/μL, Sau polymerase, 25 ng/μL T4 gp32 protein (NEB), 6.25 ng UvrD helicase, and varying amounts of DNA target input. 20 μL reactions were incubated for 2-6 hours at 37° C. on a Biotek plate reader with kinetic measurements taken either every 2.5 or 5 minutes.

### Digital Droplet PCR Quantification of Input DNA

DNA and RNA dilution series used as input target for one-pot SHERLOCK-RPA amplification reactions were quantified separately using Droplet Digital PCR (BioRad), as described before^{1,2}. Briefly, ddPCR probes were ordered from IDT PrimeTime qPCR probes with a quenched FAM/ZEN reporter. Dilution series were mixed with either (for DNA) BioRad's Supermix for Probes (no dUTP) or with (for RNA) BioRad's One-Step RT-ddPCR Advanced Kit for Probes and the corresponding qPCR probe for the target sequence. The QX200 droplet generator (BioRad) was used to generate droplets; after transferring to a droplet digital PCR plate (BioRad), thermal cycling was carried out with conditions as described in the BioRad protocol (with the exception of the Ea175 target, for which the annealing temperature was lowered according to the lower melting temperature of the primer set). Concentrations were measured using a QX200 droplet reader (Rare Event Detection, RED).

### Analysis of SHERLOCK Fluorescence Data

Fluorescent measurements were analyzed as described previously^{1,2}. Background subtracted fluorescence was calculated by subtracting the initial measured fluorescence. All reactions were run with at least three technical replicates and a control condition containing no target input.

### Analysis of Lateral Flow Results

Acquired images were converted to 8-bit grayscale using photoshop and then imported into ImageLab software (BioRad Image Lab Software 6.0.1). Images were inverted and lanes were manually adjusted to fit the lateral flow strips. Bands were picked automatically and the background was adjusted manually to allow band comparison. Width of bands and background adjustment was kept constant between all bands in the same image.

Predictive Model of Cas13 crRNA Activity

Guide activity values from the Cas13 detection tiling experiments were pre-processed by background subtracting the zero time-point fluorescence from the terminal fluorescence value. On a per-target basis, these values were further normalized to the max or median value or used as raw fluorescence values. Training was performed using a series of thresholds to classify guides into two classes (good or bad) and the best threshold was selected based on model performance. Separately, performance was also compared to separating guides into two classes based on being in the top quintile per target (good guides). For each protein (LwaCas13a or CcaCas13b), the best guide classification method was selected based on model performance.

To generate features for each guide, one-hot encoding was used to represent mono-nucleotide and di-nucleotide base identities across the guide and flanking sequence in the target. The flanking sequence length was an additional variable that was determined by measuring model performance across different flanking sequence lengths. Additional features used were normalized positions of the guide in the target and the GC content of the guide.

Logistic regressions were tested across the variable guide classification methods, flanking sequence lengths, logistic regulation tuning parameters, and regularization methods (L1 and L2). Training was performed by separating the training set into three smaller sets for training, testing, and validation. After performing three-fold cross validation on the train and test sets, a final validation of the best model was used to generate AUC curves and assay final model performance. The best performing models were then selected for the LwaCas13a and CcaCas13b datasets.

### In Vivo Knockdown Experiments

To evaluate the in vivo predictive performance of the LwaCas13a guide design model, Applicants tested guide knockdown in mammalian cell culture. Knockdown experiments were performed in HEK293FT cells (American Type Culture Collection (ATCC)), which were grown in Dulbecco's Modified Eagle Medium with high glucose, sodium pyruvate, and GlutaMAX (Thermo Fisher Scientific), additionally supplemented with lx penicillin-streptomycin (Thermo Fisher Scientific) and 10% fetal bovine serum (VWR Seradigm). Twenty-four hours prior to transfection, cells were plated at 20,000 cells per well in 96-well poly-D-lysine plates (BD Biocoat). When cells reached −90% confluency, 150 ng of LwaCas13 plasmid, 300 ng of guide expression plasmid, and 40 ng of luciferase reporter plasmid were transfected using Lipofectamine 2000 (Thermo Fisher Scientific). Plasmids were combined in Opti-MEM I Reduced Serum Medium (Thermo Fisher) to a total of 25 μL and added to 25 μL of a 2% Lipofectamine 2000 mixture in Opti-MEM. After incubation for 10 minutes, the plasmid Lipofectamine solutions were added to cells. At 48 hours post transfection, supernatant was harvested to measure secreted Gaussia luciferase and Cypridina luciferase levels using assay kits (Targeting Systems) on a plate reader (Biotek Synergy Neo 2) with an injection protocol. All replicates performed are biological replicates.

Sample Collection and Acquisition from Patients with PML-RARa and BCR-ABL Fusions

Cryopreserved bone marrow samples were obtained from the Pasquerello Tissue Bank at the Dana-Farber Cancer Institute following database query for samples harboring the PML-RARa and BCR-ABL fusion transcripts. Fresh peripheral blood and bone marrow aspirate was also obtained from 3 newly diagnosed patients (samples 1, 12, 15). All patients from whom samples were obtained had consented to the institutional tissue banking IRB protocol.

Extraction of RNA from Patient Samples with PML-RARa and BCR-ABL Fusions

Cryopreserved samples were washed with PBS and pelleted. Fresh samples (samples 1, 12, 15) collected in EDTA tubes were first treated with RBC Lysis Buffer (BD Pharmlyse) followed by PBS washes and pelleted. RNA was then extracted using the Qiagen RNeasy Kit. RNA concentrations are shown in table 8.

RT-PCR validation of PML-RARa and BCR-ABL Transcripts

cDNA was generated from 0.2-1 ug of RNA per sample using the Qiagen Quantitect Reverse Transcription kit. Nested PCR was performed using the previously validated, target specific primers and protocol described in van Dongen et al.²⁸. Primer sequences are in table 9. PCR products were visualized on a 2.5% agarose gel, shown in FIG. 13A-13D. Expected Band Sizes with nested primer sets: PML-RARa Intron 6 (214 bp); PML-RARa Intron 3 (289 bp); BCR-ABL p210 e14a2 (360 bp); BCR-ABL p210 e13a2 (285 bp); BCR-ABL p190 (e1a2: 381 bp). Note that samples with exon 6 breakpoint will have variable size bands depending on the position of breakpoint: for example, multiple bands are present in samples 4-6 (FIG. 14). GAPDH was run as a control (FIG. 14) with an expected band size of 138 bp.

Design of crRNA Targeting APML and BCR-ABL Fusion Transcripts with SHERLOCK Guide Model

Best and worst guides were predicted using the guide design web tool (sherlock.genome-engineering.org) for LwaCas13a and CcaCas13b guide design published in this study. For validation of the guide design tool, crRNAs tiling along the fusion transcript were also synthesized and tested for collateral activity (data reported in FIGS. 2, 8, and 15). The best predicted guides were used in detection of PML-RARa and BCR-ABL fusion transcripts in SHERLOCK detection assays described below.

Detection of APML and BCR-ABL Clinical RNA Ssamples with SHERLOCK

Two-step SHERLOCK assays were performed as previously described with slight modifications to the RPA protocol^{1,2}. In brief, basic RPA reactions were performed with the TwistAmp® Basic (TwistDx) protocol modified to perform RT-RPA with the following changes: 10 units/uL of AMV-RT was added after resuspension of pellet and addition of primers, following which 280 mM MgAc was added, all prior to input DNA. RT-RPA reactions at a total volume of 11 uL were run with 1 μL of input RNA for 45 minutes at 42° C. RT-RPA reactions for each fusion transcript were performed with all primer sets for all three transcripts detected in this study (PML-RARa Intron/Exon 6; PML-RARa Intron 3; BCR-ABL p210 b3a2).

Cas13 detection reactions were performed as described above with LwaCas13a and the best guide determined with the machine learning model, with the exception that reactions with a final volume of 20 uL contained 0.5 uL of input from RPA reactions. Reactions were supplemented with either RNAse Alert v2 (Invitrogen) for fluorescent readout, or a FAM-RNA-biotin reporter for lateral flow readout; reactions were incubated and quantified as described above respectively.

The initial set of samples (samples 1-11, 13-14, 16-19) were blinded for both steps of SHERLOCK detection; samples 12 and 15 were run as separate experiments as new patient samples became available. Data for both fluorescence and lateral flow were normalized to make the combined figures shown in FIG. 5 by subtracting the readout of a control reaction (RPA reaction with water input) for each experiment to include both blinded and non-blinded samples.

Two-pot SHERLOCK-RPA multiplexed lateral flow reactions were carried out as described above, with the exception reporter concentrations were lowered to a final concentration of 1 uM LwaCas13a reporter and 250 nM CcaCas13b reporter (see Table 5 for all reporters). 20 μL reactions were suspended in 100 μL of HybriDetect 1 assay buffer (Milenia) and run on custom multiplexed strips (DCN Diagnostics), and were visualized and quantified as described above.

TABLE 1
Guide RNA sequences used in this study

| | | | | | Direct | | 1st |
|---|---|---|---|---|---|---|---|
| Fig | Name | Ortholog | Complete crRNA sequence | Spacer | repeat | Target | Fig. |
| 7a | dengue_0 | LwaCas13a | GATTTAGACTACCCCAAAA | ttgagaggtt | GATTTAGACTAC | Dengue | 7a |
| | 0 | | ACGAAGGGGACTAAAACttg | ggcccctga | CCCAAAAACGAA | ssRNA | |
| | | | agaggttggcccctgaatatgtact | atatgtact | GGGGACTAAAAC | | |
| | | | (SEQ ID. NO: 12) | (SEQ ID. | (SEQ ID. | | |
| | | | | NO: 13) | NO: 14) | | |
| 7a | dengue_0 | LwaCas13a | GATTTAGACTACCCCAAAA | gttgagaggt | GATTTAGACTAC | Dengue | 7a |
| | 1 | | ACGAAGGGGACTAAAACgtt | tggcccctg | CCCAAAAACGAA | ssRNA | |
| | | | gagaggttggcccctgaatatgtac | aatatgtac | GGGGACTAAAAC | | |
| | | | (SEQ ID. NO: 15) | (SEQ ID. | (SEQ ID. | | |
| | | | | NO: 16) | NO: 17) | | |
| 7a | dengue_0 | LwaCas13a | GATTTAGACTACCCCAAAA | tgttgagagg | GATTTAGACTAC | Dengue | 7a |
| | 2 | | ACGAAGGGGACTAAAACtgt | ttggcccctg | CCCAAAAACGAA | ssRNA | |
| | | | tgagaggttggcccctgaatatgta | aatatgta | GGGGACTAAAAC | | |
| | | | (SEQ ID. NO: 18) | (SEQ ID. | (SEQ ID. | | |
| | | | | NO: 19) | NO: 20) | | |
| 7a | dengue_0 | LwaCas13a | GATTTAGACTACCCCAAAA | ttgttgagag | GATTTAGACTAC | Dengue | 7a |
| | 3 | | ACGAAGGGGACTAAAACttg | gttggcccct | CCCAAAAACGAA | ssRNA | |
| | | | ttgagaggttggcccctgaatatgt | gaatatgt | GGGGACTAAAAC | | |
| | | | (SEQ ID. NO: 21) | (SEQ ID. | (SEQ ID. | | |
| | | | | NO: 22) | NO: 23) | | |
| 7a | dengue_0 | LwaCas13a | GATTTAGACTACCCCAAAA | attgttgaga | GATTTAGACTAC | Dengue | 7a |
| | 4 | | ACGAAGGGGACTAAAACatt | ggttggccc | CCCAAAAACGAA | ssRNA | |
| | | | gttgagaggttggcccctgaatatg | ctgaatatg | GGGGACTAAAAC | | |
| | | | (SEQ ID. NO: 24) | (SEQ ID. | (SEQ ID. | | |
| | | | | NO: 25) | NO: 26) | | |
| 7a | dengue_0 | LwaCas13a | GATTTAGACTACCCCAAAA | cattgttgag | GATTTAGACTAC | Dengue | 7a |
| | 5 | | ACGAAGGGGACTAAAACcat | aggttggcc | CCCAAAAACGAA | ssRNA | |
| | | | tgttgagaggttggcccctgaatat | cctgaatat | GGGGACTAAAAC | | |
| | | | (SEQ ID. NO: 27) | (SEQ ID. | (SEQ ID. | | |
| | | | | NO: 28) | NO: 29) | | |
| 7a | dengue_0 | LwaCas13a | GATTTAGACTACCCCAAAA | tcattgttgag | GATTTAGACTAC | Dengue | 7a |
| | 6 | | ACGAAGGGGACTAAAACtca | aggttggcc | CCCAAAAACGAA | ssRNA | |
| | | | ttgttgagaggttggcccctgaata | cctgaata | GGGGACTAAAAC | | |
| | | | (SEQ ID. NO: 30) | (SEQ ID. | (SEQ ID. | | |
| | | | | NO: 31) | NO: 32) | | |
| 7a | dengue_0 | LwaCas13a | GATTTAGACTACCCCAAAA | gtcattgttga | GATTTAGACTAC | Dengue | 7a |
| | 7 | | ACGAAGGGGACTAAAACgtc | gaggttggc | CCCAAAAACGAA | ssRNA | |
| | | | attgttgagaggttggcccctgaat | ccctgaat | GGGGACTAAAAC | | |
| | | | (SEQ ID. NO: 33) | (SEQ ID. | (SEQ ID. | | |
| | | | | NO: 34) | NO: 35) | | |
| 7a | dengue_0 | LwaCas13a | GATTTAGACTACCCCAAAA | cgtcattgttg | GATTTAGACTAC | Dengue | 7a |
| | 8 | | ACGAAGGGGACTAAAACcgt | agaggttgg | CCCAAAAACGAA | ssRNA | |
| | | | cattgttgagaggttggcccctgaa | cccctgaa | GGGGACTAAAAC | | |
| | | | (SEQ ID. NO: 36) | (SEQ ID. | (SEQ ID. | | |
| | | | | NO: 37) | NO: 38) | | |
| 7a | dengue_0 | LwaCas13a | GATTTAGACTACCCCAAAA | tcgtcattgtt | GATTTAGACTAC | Dengue | 7a |
| | 9 | | ACGAAGGGGACTAAAACtcg | gagaggttg | CCCAAAAACGAA | ssRNA | |
| | | | tcattgttgagaggttggcccctga | gcccctga | GGGGACTAAAAC | | |
| | | | (SEQ ID. NO: 39) | (SEQ ID. | (SEQ ID. | | |
| | | | | NO: 40) | NO: 41) | | |
| 7a | dengue_1 | LwaCas13a | GATTTAGACTACCCCAAAA | ttcgtcattgt | GATTTAGACTAC | Dengue | 7a |
| | 0 | | ACGAAGGGGACTAAAACttc | tgagaggttg | CCCAAAAACGAA | ssRNA | |
| | | | gtcattgttgagaggttggcccctg | gcccctg | GGGGACTAAAAC | | |
| | | | (SEQ ID. NO: 42) | (SEQ ID. | (SEQ ID. | | |
| | | | | NO: 43) | NO: 44) | | |
| 7a | dengue_1 | LwaCas13a | GATTTAGACTACCCCAAAA | cttcgtcattg | GATTTAGACTAC | Dengue | 7a |
| | 1 | | ACGAAGGGGACTAAAACctt | ttgagaggtt | CCCAAAAACGAA | ssRNA | |
| | | | cgtcattgttgagaggttggcccct | ggcccct | GGGGACTAAAAC | | |
| | | | (SEQ ID. NO: 45) | (SEQ ID. | (SEQ ID. | | |
| | | | | NO: 46) | NO: 47) | | |
| S1a | dengue_1 | LwaCas13a | GATTTAGACTACCCCAAAA | tcttcgtcatt | GATTTAGACTAC | Dengue | 7a |
| | 2 | | ACGAAGGGGACTAAAACtct | gttgagaggt | CCCAAAAACGAA | ssRNA | |
| | | | tcgtcattgttgagaggttggcccc | tggcccc | GGGGACTAAAAC | | |
| | | | (SEQ ID. NO: 48) | (SEQ ID. | (SEQ ID. | | |
| | | | | NO: 49) | NO: 50) | | |
| 7a | dengue_1 | LwaCas13a | GATTTAGACTACCCCAAAA | gtcttcgtcat | GATTTAGACTAC | Dengue | 7a |
| | 3 | | ACGAAGGGGACTAAAACgtc | tgttgagagg | CCCAAAAACGAA | ssRNA | |
| | | | ttcgtcattgttgagaggttggccc | ttggccc | GGGGACTAAAAC | | |
| | | | (SEQ ID. NO: 51) | (SEQ ID. | (SEQ ID. | | |
| | | | | NO: 52) | NO: 53) | | |
| 7a | dengue_1 | LwaCas13a | GATTTAGACTACCCCAAAA | ggtcttcgtc | GATTTAGACTAC | Dengue | 7a |
| | 4 | | ACGAAGGGGACTAAAACggt | attgttgaga | CCCAAAAACGAA | ssRNA | |
| | | | cttcgtcattgttgagaggttggcc | ggttggcc | GGGGACTAAAAC | | |
| | | | (SEQ ID. NO: 54) | (SEQ ID. | (SEQ ID. | | |
| | | | | NO: 55) | NO: 56) | | |
| 7a | dengue_1 | LwaCas13a | GATTTAGACTACCCCAAAA | tggtcttcgtc | GATTTAGACTAC | Dengue | 7a |
| | 5 | | ACGAAGGGGACTAAAACtgg | attgttgaga | CCCAAAAACGAA | ssRNA | |
| | | | tcttcgtcattgttgagaggttggc | ggttggc | GGGGACTAAAAC | | |
| | | | (SEQ ID. NO: 57) | (SEQ ID. | (SEQ ID. | | |
| | | | | NO: 58) | NO: 59) | | |
| 7a | dengue_1 | LwaCas13a | GATTTAGACTACCCCAAAA | atggtcttcgt | GATTTAGACTAC | Dengue | 7a |
| | 6 | | ACGAAGGGGACTAAAACatg | cattgttgag | CCCAAAAACGAA | ssRNA | |
| | | | gtcttcgtcattgttgagaggttgg | aggttgg | GGGGACTAAAAC | | |
| | | | (SEQ ID. NO: 60) | (SEQ ID. | (SEQ ID. | | |
| | | | | NO: 61) | NO: 62) | | |
| 7a | dengue_1 | LwaCas13a | GATTTAGACTACCCCAAAA | catggtcttc | GATTTAGACTAC | Dengue | 7a |
| | 7 | | ACGAAGGGGACTAAAACcat | gtcattgttga | CCCAAAAACGAA | ssRNA | |
| | | | ggtcttcgtcattgttgagaggttg | gaggttg | GGGGACTAAAAC | | |
| | | | (SEQ ID. NO: 63) | (SEQ ID. | (SEQ ID. | | |
| | | | | NO: 64) | NO: 65) | | |
| 7a | dengue_1 | LwaCas13a | GATTTAGACTACCCCAAAA | gcatggtctt | GATTTAGACTAC | Dengue | 7a |
| | 8 | | ACGAAGGGGACTAAAACgca | cgtcattgttg | CCCAAAAACGAA | ssRNA | |
| | | | tggtcttcgtcattgttgagaggtt | agaggtt | GGGGACTAAAAC | | |
| | | | (SEQ ID. NO: 66) | (SEQ ID. | (SEQ ID. | | |
| | | | | NO: 67) | NO: 68) | | |
| 7a | dengue_1 | LwaCas13a | GATTTAGACTACCCCAAAA | agcatggtct | GATTTAGACTAC | Dengue | 7a |
| | 9 | | ACGAAGGGGACTAAAACagc | tcgtcattgtt | CCCAAAAACGAA | ssRNA | |
| | | | atggtcttcgtcattgttgagaggt | gagaggt | GGGGACTAAAAC | | |
| | | | (SEQ ID. NO: 69) | (SEQ ID. | (SEQ ID. | | |
| | | | | NO: 70) | NO: 71) | | |
| 7a | dengue_2 | LwaCas13a | GATTTAGACTACCCCAAAA | tgagcatggt | GATTTAGACTAC | Dengue | 7a |
| | 0 | | ACGAAGGGGACTAAAACtga | cttcgtcattg | CCCAAAAACGAA | ssRNA | |
| | | | gcatggtcttcgtcattgttgagag | ttgagag | GGGGACTAAAAC | | |
| | | | (SEQ ID. NO: 72) | (SEQ ID. | (SEQ ID. | | |
| | | | | NO: 73) | NO: 74) | | |
| 7a | dengue_2 | LwaCas13a | GATTTAGACTACCCCAAAA | agtgagcat | GATTTAGACTAC | Dengue | 7a |
| | 1 | | ACGAAGGGGACTAAAACagt | ggtcttcgtc | CCCAAAAACGAA | ssRNA | |
| | | | gagcatggtcttcgtcattgttgag | attgttgag | GGGGACTAAAAC | | |
| | | | (SEQ ID. NO: 75) | (SEQ ID. | (SEQ ID. | | |
| | | | | NO: 76) | NO: 77) | | |
| 7a | dengue_2 | LwaCas13a | GATTTAGACTACCCCAAAA | ccagtgagc | GATTTAGACTAC | Dengue | 7a |
| | 2 | | ACGAAGGGGACTAAAACcca | atggtcttcgt | CCCAAAAACGAA | ssRNA | |
| | | | gtgagcatggtcttcgtcattgttg | cattgttg | GGGGACTAAAAC | | |
| | | | (SEQ ID. NO: 78) | (SEQ ID. | (SEQ ID. | | |
| | | | | NO: 79) | NO: 80) | | |
| 7a | dengue_2 | LwaCas13a | GATTTAGACTACCCCAAAA | gtccagtga | GATTTAGACTAC | Dengue | 7a |
| | 3 | | ACGAAGGGGACTAAAACgtc | gcatggtctt | CCCAAAAACGAA | ssRNA | |
| | | | cagtgagcatggtcttcgtcattgt | cgtcattgt | GGGGACTAAAAC | | |
| | | | (SEQ ID. NO: 81) | (SEQ ID. | (SEQ ID. | | |
| | | | | NO: 82) | NO: 83) | | |
| 7a | dengue_2 | LwaCas13a | GATTTAGACTACCCCAAAA | ctgtccagtg | GATTTAGACTAC | Dengue | 7a |
| | 4 | | ACGAAGGGGACTAAAACctg | agcatggtct | CCCAAAAACGAA | ssRNA | |
| | | | tccagtgagcatggtcttcgtcatt | tcgtcatt | GGGGACTAAAAC | | |
| | | | (SEQ ID. NO: 84) | (SEQ ID. | (SEQ ID. | | |
| | | | | NO: 85) | NO: 86) | | |
| 7a | dengue_2 | LwaCas13a | GATTTAGACTACCCCAAAA | ttctgtccagt | GATTTAGACTAC | Dengue | 7a |
| | 5 | | ACGAAGGGGACTAAAACttc | gagcatggt | CCCAAAAACGAA | ssRNA | |
| | | | tgtccagtgagcatggtcttcgtca | cttcgtca | GGGGACTAAAAC | | |
| | | | (SEQ ID. NO: 87) | (SEQ ID. | (SEQ ID. | | |
| | | | | NO: 88) | NO: 89) | | |
| 7a | dengue_2 | LwaCas13a | GATTTAGACTACCCCAAAA | gcttctgtcc | GATTTAGACTAC | Dengue | 7a |
| | 6 | | ACGAAGGGGACTAAAACgct | agtgagcat | CCCAAAAACGAA | ssRNA | |
| | | | tctgtccagtgagcatggtcttcgt | ggtcttcgt | GGGGACTAAAAC | | |
| | | | (SEQ ID. NO: 90) | (SEQ ID. | (SEQ ID. | | |
| | | | | NO: 91) | NO: 92) | | |
| 7a | dengue_2 | LwaCas13a | GATTTAGACTACCCCAAAA | ttgcttctgtc | GATTTAGACTAC | Dengue | 7a |
| | 7 | | ACGAAGGGGACTAAAACttg | cagtgagca | CCCAAAAACGAA | ssRNA | |
| | | | cttctgtccagtgagcatggtcttc | tggtcttc | GGGGACTAAAAC | | |
| | | | (SEQ ID. NO: 93) | (SEQ ID. | (SEQ ID. | | |
| | | | | NO: 94) | NO: 95) | | |
| 7a | dengue_2 | LwaCas13a | GATTTAGACTACCCCAAAA | ttttgcttctg | GATTTAGACTAC | Dengue | 7a |
| | 8 | | ACGAAGGGGACTAAAACtttt | tccagtgagc | CCCAAAAACGAA | ssRNA | |
| | | | gcttctgtccagtgagcatggtct | atggtct | GGGGACTAAAAC | | |
| | | | (SEQ ID. NO: 96) | (SEQ ID. | (SEQ ID. | | |
| | | | | NO: 97) | NO: 98) | | |
| 7a | dengue_2 | LwaCas13a | GATTTAGACTACCCCAAAA | atttttgcttc | GATTTAGACTAC | Dengue | 7a |
| | 9 | | ACGAAGGGGACTAAAACatt | tgtccagtga | CCCAAAAACGAA | ssRNA | |
| | | | tttgcttctgtccagtgagcatggt | gcatggt | GGGGACTAAAAC | | |
| | | | (SEQ ID. NO: 99) | (SEQ ID. | (SEQ ID. | | |
| | | | | NO: 100) | NO: 101) | | |
| 7a | dengue_3 | LwaCas13a | GATTTAGACTACCCCAAAA | gcatttttgct | GATTTAGACTAC | Dengue | 7a |
| | 0 | | ACGAAGGGGACTAAAACgca | tctgtccagt | CCCAAAAACGAA | ssRNA | |
| | | | tttttgcttctgtccagtgagcatg | gagcatg | GGGGACTAAAAC | | |
| | | | (SEQ ID. NO: 102) | (SEQ ID. | (SEQ ID. | | |
| | | | | NO: 103) | NO: 104) | | |
| 7a | dengue_3 | LwaCas13a | GATTTAGACTACCCCAAAA | cagcatttttg | GATTTAGACTAC | Dengue | 7a |
| | 1 | | ACGAAGGGGACTAAAACcag | cttctgtcca | CCCAAAAACGAA | ssRNA | |
| | | | catttttgcttctgtccagtgagca | gtgagca | GGGGACTAAAAC | | |
| | | | (SEQ ID. NO: 105) | (SEQ ID. | (SEQ ID. | | |
| | | | | NO: 106) | NO: 107) | | |
| 7a | dengue_3 | LwaCas13a | GATTTAGACTACCCCAAAA | agcagcattt | GATTTAGACTAC | Dengue | 7a |
| | 2 | | ACGAAGGGGACTAAAACagc | ttgcttctgtc | CCCAAAAACGAA | ssRNA | |
| | | | agcatttttgcttctgtccagtgag | cagtgag | GGGGACTAAAAC | | |
| | | | (SEQ ID. NO: 108) | (SEQ ID. | (SEQ ID. | | |
| | | | | NO: 109) | NO: 110) | | |
| 7a | dengue_3 | LwaCas13a | GATTTAGACTACCCCAAAA | ccagcagca | GATTTAGACTAC | Dengue | 7a |
| | 3 | | ACGAAGGGGACTAAAACcca | tttttgcttct | CCCAAAAACGAA | ssRNA | |
| | | | gcagcatttttgcttctgtccagtg | gtccagtg | GGGGACTAAAAC | | |
| | | | (SEQ ID. NO: 111) | (SEQ ID. | (SEQ ID. | | |
| | | | | NO: 112) | NO: 113) | | |
| 7a | dengue_3 | LwaCas13a | GATTTAGACTACCCCAAAA | gtccagcag | GATTTAGACTAC | Dengue | 7a |
| | 4 | | ACGAAGGGGACTAAAACgtc | catttttgctt | CCCAAAAACGAA | ssRNA | |
| | | | cagcagcatttttgcttctgtccag | ctgtccag | GGGGACTAAAAC | | |
| | | | (SEQ ID. NO: 114) | (SEQ ID. | (SEQ ID. | | |
| | | | | NO: 115) | NO: 116) | | |
| 7a | dengue_3 | LwaCas13a | GATTTAGACTACCCCAAAA | ttgtccagca | GATTTAGACTAC | Dengue | 7a |
| | 5 | | ACGAAGGGGACTAAAACttg | gcatttttgct | CCCAAAAACGAA | ssRNA | |
| | | | tccagcagcatttttgcttctgtcc | tctgtcc | GGGGACTAAAAC | | |
| | | | (SEQ ID. NO: 117) | (SEQ ID. | (SEQ ID. | | |
| | | | | NO: 118) | NO: 119) | | |
| 7a | dengue_3 | LwaCas13a | GATTTAGACTACCCCAAAA | tgttgtccag | GATTTAGACTAC | Dengue | 7a |
| | 6 | | ACGAAGGGGACTAAAACtgt | cagcatttttg | CCCAAAAACGAA | ssRNA | |
| | | | tgtccagcagcatttttgcttctgt | cttctgt | GGGGACTAAAAC | | |
| | | | (SEQ ID. NO: 120) | (SEQ ID. | (SEQ ID. | | |
| | | | | NO: 121) | NO: 122) | | |
| 7a | dengue_3 | LwaCas13a | GATTTAGACTACCCCAAAA | gatgttgtcc | GATTTAGACTAC | Dengue | 7a |
| | 7 | | ACGAAGGGGACTAAAACgat | agcagcattt | CCCAAAAACGAA | ssRNA | |
| | | | gttgtccagcagcatttttgcttct | ttgcttct | GGGGACTAAAAC | | |
| | | | (SEQ ID. NO: 123) | (SEQ ID. | (SEQ ID. | | |
| | | | | NO: 124) | NO: 125) | | |
| 7a | dengue_3 | LwaCas13a | GATTTAGACTACCCCAAAA | ttgatgttgtc | GATTTAGACTAC | Dengue | 7a |
| | 8 | | ACGAAGGGGACTAAAACttg | cagcagcatt | CCCAAAAACGAA | ssRNA | |
| | | | atgttgtccagcagcatttttgctt | tttgctt | GGGGACTAAAAC | | |
| | | | (SEQ ID. NO: 126) | (SEQ ID. | (SEQ ID. | | |
| | | | | NO: 127) | NO: 128) | | |
| 7a | dengue_3 | LwaCas13a | GATTTAGACTACCCCAAAA | tgttgatgttg | GATTTAGACTAC | Dengue | 7a |
| | 9 | | ACGAAGGGGACTAAAACtgt | tccagcagc | CCCAAAAACGAA | ssRNA | |
| | | | tgatgttgtccagcagcatttttgc | atttttgc | GGGGACTAAAAC | | |
| | | | (SEQ ID. NO: 129) | (SEQ ID. | (SEQ ID. | | |
| | | | | NO: 130) | NO: 131) | | |
| 7a | dengue_4 | LwaCas13a | GATTTAGACTACCCCAAAA | tgtgttgatgt | GATTTAGACTAC | Dengue | 7a |
| | 0 | | ACGAAGGGGACTAAAACtgt | tgtccagca | CCCAAAAACGAA | ssRNA | |
| | | | gttgatgttgtccagcagcattttt | gcattttt | GGGGACTAAAAC | | |
| | | | (SEQ ID. NO: 132) | (SEQ ID. | (SEQ ID. | | |
| | | | | NO: 133) | NO: 134) | | |
| 7a | dengue_4 | LwaCas13a | GATTTAGACTACCCCAAAA | ggtgtgttga | GATTTAGACTAC | Dengue | 7a |
| | 1 | | ACGAAGGGGACTAAAACggt | tgttgtccag | CCCAAAAACGAA | ssRNA | |
| | | | gtgttgatgttgtccagcagcattt | cagcattt | GGGGACTAAAAC | | |
| | | | (SEQ ID. NO: 135) | (SEQ ID. | (SEQ ID. | | |
| | | | | NO: 136) | NO: 137) | | |
| 7a | dengue_4 | LwaCas13a | GATTTAGACTACCCCAAAA | ctggtgtgtt | GATTTAGACTAC | Dengue | 7a |
| | 2 | | ACGAAGGGGACTAAAACctg | gatgttgtcc | CCCAAAAACGAA | ssRNA | |
| | | | gtgtgttgatgttgtccagcagcat | agcagcat | GGGGACTAAAAC | | |
| | | | (SEQ ID. NO: 138) | (SEQ ID. | (SEQ ID. | | |
| | | | | NO: 139) | NO: 140) | | |
| 7a | dengue_4 | LwaCas13a | GATTTAGACTACCCCAAAA | ttctggtgtgt | GATTTAGACTAC | Dengue | 7a |
| | 3 | | ACGAAGGGGACTAAAACttc | tgatgttgtcc | CCCAAAAACGAA | ssRNA | |
| | | | tggtgtgttgatgttgtccagcagc | agcagc | GGGGACTAAAAC | | |
| | | | (SEQ ID. NO: 141) | (SEQ ID. | (SEQ ID. | | |
| | | | | NO: 142) | NO: 143) | | |
| 7a | dengue_4 | LwaCas13a | GATTTAGACTACCCCAAAA | ccttctggtgt | GATTTAGACTAC | Dengue | 7a |
| | 4 | | ACGAAGGGGACTAAAACcct | gttgatgttgt | CCCAAAAACGAA | ssRNA | |
| | | | tctggtgtgttgatgttgtccagca | ccagca | GGGGACTAAAAC | | |
| | | | (SEQ ID. NO: 144) | (SEQ ID. | (SEQ ID. | | |
| | | | | NO: 145) | NO: 146) | | |
| 7a | dengue_4 | LwaCas13a | GATTTAGACTACCCCAAAA | tcccttctggt | GATTTAGACTAC | Dengue | 7a |
| | 5 | | ACGAAGGGGACTAAAACtcc | gtgttgatgtt | CCCAAAAACGAA | ssRNA | |
| | | | cttctggtgtgttgatgttgtccag | gtccag | GGGGACTAAAAC | | |
| | | | (SEQ ID. NO: 147) | (SEQ ID. | (SEQ ID. | | |
| | | | | NO: 148) | NO: 149) | | |
| 7a | dengue_4 | LwaCas13a | GATTTAGACTACCCCAAAA | aatcccttct | GATTTAGACTAC | Dengue | 7a |
| | 6 | | ACGAAGGGGACTAAAACaat | ggtgtgttga | CCCAAAAACGAA | ssRNA | |
| | | | cccttctggtgtgttgatgttgtcc | tgttgtcc | GGGGACTAAAAC | | |
| | | | (SEQ ID. NO: 150) | (SEQ ID. | (SEQ ID. | | |
| | | | | NO: 151) | NO: 152) | | |
| 7a | dengue_4 | LwaCas13a | GATTTAGACTACCCCAAAA | ataatcccttc | GATTTAGACTAC | Dengue | 7a |
| | 7 | | ACGAAGGGGACTAAAACata | tggtgtgttg | CCCAAAAACGAA | ssRNA | |
| | | | atcccttctggtgtgttgatgttgt | atgttgt | GGGGACTAAAAC | | |
| | | | (SEQ ID. NO: 153) | (SEQ ID. | (SEQ ID. | | |
| | | | | NO: 154) | NO: 155) | | |
| 7a | dengue_4 | LwaCas13a | GATTTAGACTACCCCAAAA | gtataatccc | GATTTAGACTAC | Dengue | 7a |
| | 8 | | ACGAAGGGGACTAAAACgta | ttctggtgtgt | CCCAAAAACGAA | ssRNA | |
| | | | taatcccttctggtgtgttgatgtt | tgatgtt | GGGGACTAAAAC | | |
| | | | (SEQ ID. NO: 156) | (SEQ ID. | (SEQ ID. | | |
| | | | | NO: 157) | NO: 158) | | |
| 7a | dengue_4 | LwaCas13a | GATTTAGACTACCCCAAAA | tggtataatc | GATTTAGACTAC | Dengue | 7a |
| | 9 | | ACGAAGGGGACTAAAACtgg | ccttctggtgt | CCCAAAAACGAA | ssRNA | |
| | | | tataatcccttctggtgtgttgatg | gttgatg | GGGGACTAAAAC | | |
| | | | (SEQ ID. NO: 159) | (SEQ ID. | (SEQ ID. | | |
| | | | | NO: 160) | NO: 161) | | |
| 7a | dengue_5 | LwaCas13a | GATTTAGACTACCCCAAAA | gctggtataa | GATTTAGACTAC | Dengue | 7a |
| | 0 | | ACGAAGGGGACTAAAACgct | tcccttctggt | CCCAAAAACGAA | ssRNA | |
| | | | ggtataatcccttctggtgtgttga | gtgttga | GGGGACTAAAAC | | |
| | | | (SEQ ID. NO: 162) | (SEQ ID. | (SEQ ID. | | |
| | | | | NO: 163) | NO: 164) | | |
| 7a | dengue_5 | LwaCas13a | GATTTAGACTACCCCAAAA | gagctggtat | GATTTAGACTAC | Dengue | 7a |
| | 1 | | ACGAAGGGGACTAAAACgag | aatcccttct | CCCAAAAACGAA | ssRNA | |
| | | | ctggtataatcccttctggtgtgtt | ggtgtgtt | GGGGACTAAAAC | | |
| | | | (SEQ ID. NO: 165) | (SEQ ID. | (SEQ ID. | | |
| | | | | NO: 166) | NO: 167) | | |
| 7a | dengue_5 | LwaCas13a | GATTTAGACTACCCCAAAA | gagagctgg | GATTTAGACTAC | Dengue | 7a |
| | 2 | | ACGAAGGGGACTAAAACgag | tataatccctt | CCCAAAAACGAA | ssRNA | |
| | | | agctggtataatcccttctggtgtg | ctggtgtg | GGGGACTAAAAC | | |
| | | | (SEQ ID. NO: 168) | (SEQ ID. | (SEQ ID. | | |
| | | | | NO: 169) | NO: 170) | | |
| 7a | dengue_5 | LwaCas13a | GATTTAGACTACCCCAAAA | aagagagct | GATTTAGACTAC | Dengue | 7a |
| | 3 | | ACGAAGGGGACTAAAACaag | ggtataatcc | CCCAAAAACGAA | ssRNA | |
| | | | agagctggtataatcccttctggtg | cttctggtg | GGGGACTAAAAC | | |
| | | | (SEQ ID. NO: 171) | (SEQ ID. | (SEQ ID. | | |
| | | | | NO: 172) | NO: 173) | | |
| 7a | dengue_5 | LwaCas13a | GATTTAGACTACCCCAAAA | caaagagag | GATTTAGACTAC | Dengue | 7a |
| | 4 | | ACGAAGGGGACTAAAACcaa | ctggtataat | CCCAAAAACGAA | ssRNA | |
| | | | agagagctggtataatcccttctgg | cccttctgg | GGGGACTAAAAC | | |
| | | | (SEQ ID. NO: 174) | (SEQ ID. | (SEQ ID. | | |
| | | | | NO: 175) | NO: 176) | | |
| 7a | dengue_5 | LwaCas13a | GATTTAGACTACCCCAAAA | ttcaaagaga | GATTTAGACTAC | Dengue | 7a |
| | 5 | | ACGAAGGGGACTAAAACttc | gctggtataa | CCCAAAAACGAA | ssRNA | |
| | | | aaagagagctggtataatcccttct | tcccttct | GGGGACTAAAAC | | |
| | | | (SEQ ID. NO: 177) | (SEQ ID. | (SEQ ID. | | |
| | | | | NO: 178) | NO: 179) | | |
| 7a | dengue_5 | LwaCas13a | GATTTAGACTACCCCAAAA | ggttcaaag | GATTTAGACTAC | Dengue | 7a |
| | 6 | | ACGAAGGGGACTAAAACggt | agagctggt | CCCAAAAACGAA | ssRNA | |
| | | | tcaaagagagctggtataatccctt | ataatccctt | GGGGACTAAAAC | | |
| | | | (SEQ ID. NO: 180) | (SEQ ID. | (SEQ ID. | | |
| | | | | NO: 181) | NO: 182) | | |
| 7a | dengue_5 | LwaCas13a | GATTTAGACTACCCCAAAA | ctggttcaaa | GATTTAGACTAC | Dengue | 7a |
| | 7 | | ACGAAGGGGACTAAAACctg | gagagctgg | CCCAAAAACGAA | ssRNA | |
| | | | gttcaaagagagctggtataatccc | tataatccc | GGGGACTAAAAC | | |
| | | | (SEQ ID. NO: 183) | (SEQ ID. | (SEQ ID. | | |
| | | | | NO: 184) | NO: 185) | | |
| 7a | dengue_5 | LwaCas13a | GATTTAGACTACCCCAAAA | ttctggttcaa | GATTTAGACTAC | Dengue | 7a |
| | 8 | | ACGAAGGGGACTAAAACttc | agagagctg | CCCAAAAACGAA | ssRNA | |
| | | | tggttcaaagagagctggtataatc | gtataatc | GGGGACTAAAAC | | |
| | | | (SEQ ID. NO: 186) | (SEQ ID. | (SEQ ID. | | |
| | | | | NO: 187) | NO: 188) | | |
| 7a | dengue_5 | LwaCas13a | GATTTAGACTACCCCAAAA | ctttctggttc | GATTTAGACTAC | Dengue | 7a |
| | 9 | | ACGAAGGGGACTAAAACctt | aaagagagc | CCCAAAAACGAA | ssRNA | |
| | | | tctggttcaaagagagctggtataa | tggtataa | GGGGACTAAAAC | | |
| | | | (SEQ ID. NO: 189) | (SEQ ID. | (SEQ ID. | | |
| | | | | NO: 190) | NO: 191) | | |
| 7a | dengue_6 | LwaCas13a | GATTTAGACTACCCCAAAA | ccctttctggt | GATTTAGACTAC | Dengue | 7a |
| | 0 | | ACGAAGGGGACTAAAACccc | tcaaagaga | CCCAAAAACGAA | ssRNA | |
| | | | tttctggttcaaagagagctggtat | gctggtat | GGGGACTAAAAC | | |
| | | | (SEQ ID. NO: 192) | (SEQ ID. | (SEQ ID. | | |
| | | | | NO: 193) | NO: 194) | | |
| 7a | dengue_6 | LwaCas13a | GATTTAGACTACCCCAAAA | ctccctttctg | GATTTAGACTAC | Dengue | 7a |
| | 1 | | ACGAAGGGGACTAAAACctc | gttcaaaga | CCCAAAAACGAA | ssRNA | |
| | | | cctttctggttcaaagagagctggt | gagctggt | GGGGACTAAAAC | | |
| | | | (SEQ ID. NO: 195) | (SEQ ID. | (SEQ ID. | | |
| | | | | NO: 196) | NO: 197) | | |
| 7a | dengue_6 | LwaCas13a | GATTTAGACTACCCCAAAA | ttctccctttc | GATTTAGACTAC | Dengue | 7a |
| | 2 | | ACGAAGGGGACTAAAACttc | tggttcaaag | CCCAAAAACGAA | ssRNA | |
| | | | tccctttctggttcaaagagagctg | agagctg | GGGGACTAAAAC | | |
| | | | (SEQ ID. NO: 198) | (SEQ ID. | (SEQ ID. | | |
| | | | | NO: 199) | NO: 200) | | |
| 7a | dengue_6 | LwaCas13a | GATTTAGACTACCCCAAAA | acttctccctt | GATTTAGACTAC | Dengue | 7a |
| | 3 | | ACGAAGGGGACTAAAACact | tctggttcaa | CCCAAAAACGAA | ssRNA | |
| | | | tctccctttctggttcaaagagagc | agagagc | GGGGACTAAAAC | | |
| | | | (SEQ ID. NO: 201) | (SEQ ID. | (SEQ ID. | | |
| | | | | NO: 202) | NO: 203) | | |
| 7a | dengue_6 | LwaCas13a | GATTTAGACTACCCCAAAA | tgacttctcc | GATTTAGACTAC | Dengue | 7a |
| | 4 | | ACGAAGGGGACTAAAACtga | ctttctggttc | CCCAAAAACGAA | ssRNA | |
| | | | cttctccctttctggttcaaagaga | aaagaga | GGGGACTAAAAC | | |
| | | | (SEQ ID. NO: 204) | (SEQ ID. | (SEQ ID. | | |
| | | | | NO: 205) | NO: 206) | | |
| 7a | dengue_6 | LwaCas13a | GATTTAGACTACCCCAAAA | ctgacttctc | GATTTAGACTAC | Dengue | 7a |
| | 5 | | ACGAAGGGGACTAAAACctg | cctttctggtt | CCCAAAAACGAA | ssRNA | |
| | | | acttctccctttctggttcaaagag | caaagag | GGGGACTAAAAC | | |
| | | | (SEQ ID. NO: 207) | (SEQ ID. | (SEQ ID. | | |
| | | | | NO: 208) | NO: 209) | | |
| 7a | dengue_6 | LwaCas13a | GATTTAGACTACCCCAAAA | gctgacttct | GATTTAGACTAC | Dengue | 7a |
| | 6 | | ACGAAGGGGACTAAAACgct | ccctttctggt | CCCAAAAACGAA | ssRNA | |
| | | | gacttctccctttctggttcaaaga | tcaaaga | GGGGACTAAAAC | | |
| | | | (SEQ ID. NO: 210) | (SEQ ID. | (SEQ ID. | | |
| | | | | NO: 211) | NO: 212) | | |
| 7a | dengue_6 | LwaCas13a | GATTTAGACTACCCCAAAA | ggctgacttc | GATTTAGACTAC | Dengue | 7a |
| | 7 | | ACGAAGGGGACTAAAACggc | tccctttctgg | CCCAAAAACGAA | ssRNA | |
| | | | tgacttctccctttctggttcaaag | ttcaaag | GGGGACTAAAAC | | |
| | | | (SEQ ID. NO: 213) | (SEQ ID. | (SEQ ID. | | |
| | | | | NO: 214) | NO: 215) | | |
| 7a | dengue_6 | LwaCas13a | GATTTAGACTACCCCAAAA | cggctgactt | GATTTAGACTAC | Dengue | 7a |
| | 8 | | ACGAAGGGGACTAAAACcgg | ctccctttctg | CCCAAAAACGAA | ssRNA | |
| | | | ctgacttctccctttctggttcaaa | gttcaaa | GGGGACTAAAAC | | |
| | | | (SEQ ID. NO: 216) | (SEQ ID. | (SEQ ID. | | |
| | | | | NO: 217) | NO: 218) | | |
| 7a | dengue_6 | LwaCas13a | GATTTAGACTACCCCAAAA | gcggctgac | GATTTAGACTAC | Dengue | 7a |
| | 9 | | ACGAAGGGGACTAAAACgcg | ttctccctttc | CCCAAAAACGAA | ssRNA | |
| | | | gctgacttctccctttctggttcaa | tggttcaa | GGGGACTAAAAC | | |
| | | | (SEQ ID. NO: 219) | (SEQ ID. | (SEQ ID. | | |
| | | | | NO: 220) | NO: 221) | | |
| 7a | dengue_7 | LwaCas13a | GATTTAGACTACCCCAAAA | ggcggctga | GATTTAGACTAC | Dengue | 7a |
| | 0 | | ACGAAGGGGACTAAAACggc | cttctcccttt | CCCAAAAACGAA | ssRNA | |
| | | | ggctgacttctccctttctggttca | ctggttca | GGGGACTAAAAC | | |
| | | | (SEQ ID. NO: 222) | (SEQ ID. | (SEQ ID. | | |
| | | | | NO: 223) | NO: 224) | | |
| 7a | dengue_7 | LwaCas13a | GATTTAGACTACCCCAAAA | tggcggctg | GATTTAGACTAC | Dengue | 7a |
| | 1 | | ACGAAGGGGACTAAAACtgg | acttctccctt | CCCAAAAACGAA | ssRNA | |
| | | | cggctgacttctccctttctggttc | tctggttc | GGGGACTAAAAC | | |
| | | | (SEQ ID. NO: 225) | (SEQ ID. | (SEQ ID. | | |
| | | | | NO: 226) | NO: 227) | | |
| 7a | dengue_7 | LwaCas13a | GATTTAGACTACCCCAAAA | atggcggct | GATTTAGACTAC | Dengue | 7a |
| | 2 | | ACGAAGGGGACTAAAACatg | gacttctccc | CCCAAAAACGAA | ssRNA | |
| | | | gcggctgacttctccctttctggtt | tttctggtt | GGGGACTAAAAC | | |
| | | | (SEQ ID. NO: 228) | (SEQ ID. | (SEQ ID. | | |
| | | | | NO: 229) | NO: 230) | | |
| 7a | dengue_7 | LwaCas13a | GATTTAGACTACCCCAAAA | tatggcggct | GATTTAGACTAC | Dengue | 7a |
| | 3 | | ACGAAGGGGACTAAAACtat | gacttctccc | CCCAAAAACGAA | ssRNA | |
| | | | ggcggctgacttctccctttctggt | tttctggt | GGGGACTAAAAC | | |
| | | | (SEQ ID. NO: 231) | (SEQ ID. | (SEQ ID. | | |
| | | | | NO: 232) | NO: 233) | | |
| 7a | dengue_7 | LwaCas13a | GATTTAGACTACCCCAAAA | ctatggcgg | GATTTAGACTAC | Dengue | 7a |
| | 4 | | ACGAAGGGGACTAAAACcta | ctgacttctc | CCCAAAAACGAA | ssRNA | |
| | | | tggcggctgacttctccctttctgg | cctttctgg | GGGGACTAAAAC | | |
| | | | (SEQ ID. NO: 234) | (SEQ ID. | (SEQ ID. | | |
| | | | | NO: 235) | NO: 236) | | |
| 7a | dengue_7 | LwaCas13a | GATTTAGACTACCCCAAAA | tctatggcgg | GATTTAGACTAC | Dengue | 7a |
| | 5 | | ACGAAGGGGACTAAAACtct | ctgacttctc | CCCAAAAACGAA | ssRNA | |
| | | | atggcggctgacttctccctttctg | cctttctg | GGGGACTAAAAC | | |
| | | | (SEQ ID. NO: 237) | (SEQ ID. | (SEQ ID. | | |
| | | | | NO: 238) | NO: 239) | | |
| 7a | dengue_7 | LwaCas13a | GATTTAGACTACCCCAAAA | gtctatggcg | GATTTAGACTAC | Dengue | 7a |
| | 6 | | ACGAAGGGGACTAAAACgtc | gctgacttct | CCCAAAAACGAA | ssRNA | |
| | | | tatggcggctgacttctccctttct | ccctttct | GGGGACTAAAAC | | |
| | | | (SEQ ID. NO: 240) | (SEQ ID. | (SEQ ID. | | |
| | | | | NO: 241) | NO: 242) | | |
| 7a | dengue_7 | LwaCas13a | GATTTAGACTACCCCAAAA | cgtctatggc | GATTTAGACTAC | Dengue | 7a |
| | 7 | | ACGAAGGGGACTAAAACcgt | ggctgacttc | CCCAAAAACGAA | ssRNA | |
| | | | ctatggcggctgacttctccctttc | tccctttc | GGGGACTAAAAC | | |
| | | | (SEQ ID. NO: 243) | (SEQ ID. | (SEQ ID. | | |
| | | | | NO: 244) | NO: 245) | | |
| 7a | dengue_7 | LwaCas13a | GATTTAGACTACCCCAAAA | ccgtctatgg | GATTTAGACTAC | Dengue | 7a |
| | 8 | | ACGAAGGGGACTAAAACccg | cggctgactt | CCCAAAAACGAA | ssRNA | |
| | | | tctatggcggctgacttctcccttt | ctcccttt | GGGGACTAAAAC | | |
| | | | (SEQ ID. NO: 246) | (SEQ ID. | (SEQ ID. | | |
| | | | | NO: 247) | NO: 248) | | |
| 7a | dengue_7 | LwaCas13a | GATTTAGACTACCCCAAAA | accgtctatg | GATTTAGACTAC | Dengue | 7a |
| | 9 | | ACGAAGGGGACTAAAACacc | gcggctgac | CCCAAAAACGAA | ssRNA | |
| | | | gtctatggcggctgacttctccctt | ttctccctt | GGGGACTAAAAC | | |
| | | | (SEQ ID. NO: 249) | (SEQ ID. | (SEQ ID. | | |
| | | | | NO: 250) | NO: 251) | | |
| 7a | dengue_8 | LwaCas13a | GATTTAGACTACCCCAAAA | caccgtctat | GATTTAGACTAC | Dengue | 7a |
| | 0 | | ACGAAGGGGACTAAAACcac | ggcggctga | CCCAAAAACGAA | ssRNA | |
| | | | cgtctatggcggctgacttctccct | cttctccct | GGGGACTAAAAC | | |
| | | | (SEQ ID. NO: 252) | (SEQ ID. | (SEQ ID. | | |
| | | | | NO: 253) | NO: 254) | | |
| 7a | dengue_8 | LwaCas13a | GATTTAGACTACCCCAAAA | tcaccgtcta | GATTTAGACTAC | Dengue | 7a |
| | 1 | | ACGAAGGGGACTAAAACtca | tggcggctg | CCCAAAAACGAA | ssRNA | |
| | | | ccgtctatggcggctgacttctccc | acttctccc | GGGGACTAAAAC | | |
| | | | (SEQ ID. NO: 255) | (SEQ ID. | (SEQ ID. | | |
| | | | | NO: 256) | NO: 257) | | |
| 7a | dengue_8 | LwaCas13a | GATTTAGACTACCCCAAAA | ttcaccgtct | GATTTAGACTAC | Dengue | 7a |
| | 2 | | ACGAAGGGGACTAAAACttc | atggcggct | CCCAAAAACGAA | ssRNA | |
| | | | accgtctatggcggctgacttctcc | gacttctcc | GGGGACTAAAAC | | |
| | | | (SEQ ID. NO: 258) | (SEQ ID. | (SEQ ID. | | |
| | | | | NO: 259) | NO: 260) | | |
| 7a | dengue_8 | LwaCas13a | GATTTAGACTACCCCAAAA | attcaccgtc | GATTTAGACTAC | Dengue | 7a |
| | 3 | | ACGAAGGGGACTAAAACatt | tatggcggct | CCCAAAAACGAA | ssRNA | |
| | | | caccgtctatggcggctgacttctc | gacttctc | GGGGACTAAAAC | | |
| | | | (SEQ ID. NO: 261) | (SEQ ID. | (SEQ ID. | | |
| | | | | NO: 262) | NO: 263) | | |
| 7a | dengue_8 | LwaCas13a | GATTTAGACTACCCCAAAA | tattcaccgt | GATTTAGACTAC | Dengue | 7a |
| | 4 | | ACGAAGGGGACTAAAACtat | ctatggcgg | CCCAAAAACGAA | ssRNA | |
| | | | tcaccgtctatggcggctgacttct | ctgacttct | GGGGACTAAAAC | | |
| | | | (SEQ ID. NO: 264) | (SEQ ID. | (SEQ ID. | | |
| | | | | NO: 265) | NO: 266) | | |
| 7a | dengue_8 | LwaCas13a | GATTTAGACTACCCCAAAA | gtattcaccg | GATTTAGACTAC | Dengue | 7a |
| | 5 | | ACGAAGGGGACTAAAACgta | tctatggcgg | CCCAAAAACGAA | ssRNA | |
| | | | ttcaccgtctatggeggctgacttc | ctgacttc | GGGGACTAAAAC | | |
| | | | (SEQ ID. NO: 267) | (SEQ ID. | (SEQ ID. | | |
| | | | | NO: 268) | NO: 269) | | |
| 7a | dengue_8 | LwaCas13a | GATTTAGACTACCCCAAAA | ggtattcacc | GATTTAGACTAC | Dengue | 7a |
| | 6 | | ACGAAGGGGACTAAAACggt | gtctatggcg | CCCAAAAACGAA | ssRNA | |
| | | | attcaccgtctatggcggctgactt | gctgactt | GGGGACTAAAAC | | |
| | | | (SEQ ID. NO: 270) | (SEQ ID. | (SEQ ID. | | |
| | | | | NO: 271) | NO: 272) | | |
| 7a | dengue_8 | LwaCas13a | GATTTAGACTACCCCAAAA | cggtattcac | GATTTAGACTAC | Dengue | 7a |
| | 7 | | ACGAAGGGGACTAAAACcgg | cgtctatggc | CCCAAAAACGAA | ssRNA | |
| | | | tattcaccgtctatggcggctgact | ggctgact | GGGGACTAAAAC | | |
| | | | (SEQ ID. NO: 273) | (SEQ ID. | (SEQ ID. | | |
| | | | | NO: 274) | NO: 275) | | |
| 7a | dengue_8 | LwaCas13a | GATTTAGACTACCCCAAAA | geggtattca | GATTTAGACTAC | Dengue | 7a |
| | 8 | | ACGAAGGGGACTAAAACgcg | ccgtctatgg | CCCAAAAACGAA | ssRNA | |
| | | | gtattcaccgtctatggcggctgac | cggctgac | GGGGACTAAAAC | | |
| | | | (SEQ ID. NO: 276) | (SEQ ID. | (SEQ ID. | | |
| | | | | NO: 277) | NO: 278) | | |
| 7a | dengue_8 | LwaCas13a | GATTTAGACTACCCCAAAA | ggcggtattc | GATTTAGACTAC | Dengue | 7a |
| | 9 | | ACGAAGGGGACTAAAACggc | accgtctatg | CCCAAAAACGAA | ssRNA | |
| | | | ggtattcaccgtctatggcggctga | gcggctga | GGGGACTAAAAC | | |
| | | | (SEQ ID. NO: 279) | (SEQ ID. | (SEQ ID. | | |
| | | | | NO: 280) | NO: 281) | | |
| 7a | dengue_9 | LwaCas13a | GATTTAGACTACCCCAAAA | aggcggtatt | GATTTAGACTAC | Dengue | 7a |
| | 0 | | ACGAAGGGGACTAAAACagg | caccgtctat | CCCAAAAACGAA | ssRNA | |
| | | | cggtattcaccgtctatggcggctg | ggcggctg | GGGGACTAAAAC | | |
| | | | (SEQ ID. NO: 282) | (SEQ ID. | (SEQ ID. | | |
| | | | | NO: 283) | NO: 284) | | |
| 7a | dengue_9 | LwaCas13a | GATTTAGACTACCCCAAAA | caggcggta | GATTTAGACTAC | Dengue | 7a |
| | 1 | | ACGAAGGGGACTAAAACcag | ttcaccgtct | CCCAAAAACGAA | ssRNA | |
| | | | gcggtattcaccgtctatggcggct | atggcggct | GGGGACTAAAAC | | |
| | | | (SEQ ID. NO: 285) | (SEQ ID. | (SEQ ID. | | |
| | | | | NO: 286) | NO: 287) | | |
| 7a | dengue_9 | LwaCas13a | GATTTAGACTACCCCAAAA | tcaggcggt | GATTTAGACTAC | Dengue | 7a |
| | 2 | | ACGAAGGGGACTAAAACtca | attcaccgtc | CCCAAAAACGAA | ssRNA | |
| | | | ggcggtattcaccgtctatggcggc | tatggcggc | GGGGACTAAAAC | | |
| | | | (SEQ ID. NO: 288) | (SEQ ID. | (SEQ ID. | | |
| | | | | NO: 289) | NO: 290) | | |
| 7a | dengue_9 | LwaCas13a | GATTTAGACTACCCCAAAA | ttcaggcggt | GATTTAGACTAC | Dengue | 7a |
| | 3 | | ACGAAGGGGACTAAAACttc | attcaccgtc | CCCAAAAACGAA | ssRNA | |
| | | | aggcggtattcaccgtctatggcgg | tatggcgg | GGGGACTAAAAC | | |
| | | | (SEQ ID. NO: 291) | (SEQ ID. | (SEQ ID. | | |
| | | | | NO: 292) | NO: 293) | | |
| 7a | dengue_9 | LwaCas13a | GATTTAGACTACCCCAAAA | cttcaggcg | GATTTAGACTAC | Dengue | 7a |
| | 4 | | ACGAAGGGGACTAAAACctt | gtattcaccg | CCCAAAAACGAA | ssRNA | |
| | | | caggcggtattcaccgtctatggcg | tctatggcg | GGGGACTAAAAC | | |
| | | | (SEQ ID. NO: 294) | (SEQ ID. | (SEQ ID. | | |
| | | | | NO: 295) | NO: 296) | | |
| 7a | dengue_9 | LwaCas13a | GATTTAGACTACCCCAAAA | ccttcaggc | GATTTAGACTAC | Dengue | 7a |
| | 5 | | ACGAAGGGGACTAAAACcct | ggtattcacc | CCCAAAAACGAA | ssRNA | |
| | | | tcaggcggtattcaccgtctatggc | gtctatggc | GGGGACTAAAAC | | |
| | | | (SEQ ID. NO: 297) | (SEQ ID. | (SEQ ID. | | |
| | | | | NO: 298) | NO: 299) | | |
| 1b | Ebola_GP | LwaCas13a | GATTTAGACTACCCCAAAA | ctgtgaaag | GATTTAGACTAC | Ebola | 1b |
| | _guide_01 | | ACGAAGGGGACTAAAACctg | acaactcttc | CCCAAAAACGAA | ssRNA | |
| | | | tgaaagacaactcttcactgcgaat | actgcgaat | GGGGACTAAAAC | | |
| | | | (SEQ ID. NO: 300) | (SEQ ID. | (SEQ ID. | | |
| | | | | NO: 301) | NO: 302) | | |
| 1b | Ebola_GP | LwaCas13a | GATTTAGACTACCCCAAAA | gatacaactg | GATTTAGACTAC | Ebola | 1b |
| | _guide_06 | | ACGAAGGGGACTAAAACgat | tgaaagaca | CCCAAAAACGAA | ssRNA | |
| | | | acaactgtgaaagacaactcttcac | actcttcac | GGGGACTAAAAC | | |
| | | | (SEQ ID. NO: 303) | (SEQ ID. | (SEQ ID. | | |
| | | | | NO: 304) | NO: 305) | | |
| 1b | Ebola_GP | LwaCas13a | GATTTAGACTACCCCAAAA | tgatacaact | GATTTAGACTAC | Ebola | 1b |
| | _guide_07 | | ACGAAGGGGACTAAAACtga | gtgaaagac | CCCAAAAACGAA | ssRNA | |
| | | | tacaactgtgaaagacaactcttca | aactcttca | GGGGACTAAAAC | | |
| | | | (SEQ ID. NO: 306) | (SEQ ID. | (SEQ ID. | | |
| | | | | NO: 307) | NO: 308) | | |
| 1b | Ebola_GP | LwaCas13a | GATTTAGACTACCCCAAAA | tttgatacaa | GATTTAGACTAC | Ebola | 1b |
| | _guide_08 | | ACGAAGGGGACTAAAACttt | ctgtgaaag | CCCAAAAACGAA | ssRNA | |
| | | | gatacaactgtgaaagacaactctt | acaactctt | GGGGACTAAAAC | | |
| | | | (SEQ ID. NO: 309) | (SEQ ID. | (SEQ ID. | | |
| | | | | NO: 310) | NO: 311) | | |
| 1b | Ebola_GP | LwaCas13a | GATTTAGACTACCCCAAAA | tccgtttgata | GATTTAGACTAC | Ebola | 1b |
| | _guide_11 | | ACGAAGGGGACTAAAACtcc | caactgtgaa | CCCAAAAACGAA | ssRNA | |
| | | | gtttgatacaactgtgaaagacaac | agacaac | GGGGACTAAAAC | | |
| | | | (SEQ ID. NO: 312) | (SEQ ID. | (SEQ ID. | | |
| | | | | NO: 313) | NO: 314) | | |
| 1b | Ebola_GP | LwaCas13a | GATTTAGACTACCCCAAAA | ggctccgttt | GATTTAGACTAC | Ebola | 1b |
| | _guide_13 | | ACGAAGGGGACTAAAACggc | gatacaactg | CCCAAAAACGAA | ssRNA | |
| | | | tccgtttgatacaactgtgaaagac | tgaaagac | GGGGACTAAAAC | | |
| | | | (SEQ ID. NO: 315) | (SEQ ID. | (SEQ ID. | | |
| | | | | NO: 316) | NO: 317) | | |
| 1b | Ebola_GP | LwaCas13a | GATTTAGACTACCCCAAAA | ccactgatgt | GATTTAGACTAC | Ebola | 1b |
| | _guide_20 | | ACGAAGGGGACTAAAACcca | ttttggctccg | CCCAAAAACGAA | ssRNA | |
| | | | ctgatgtttttggctccgtttgata | tttgata | GGGGACTAAAAC | | |
| | | | (SEQ ID. NO: 318) | (SEQ ID. | (SEQ ID. | | |
| | | | | NO: 319) | NO: 320) | | |
| 1b | Ebola_GP | LwaCas13a | GATTTAGACTACCCCAAAA | accactgatg | GATTTAGACTAC | Ebola | 1b |
| | _guide_21 | | ACGAAGGGGACTAAAACacc | tttttggctcc | CCCAAAAACGAA | ssRNA | |
| | | | actgatgtttttggctccgtttgat | gtttgat | GGGGACTAAAAC | | |
| | | | (SEQ ID. NO: 321) | (SEQ ID. | (SEQ ID. | | |
| | | | | NO: 322) | NO: 323) | | |
| 1b | Ebola_GP | LwaCas13a | GATTTAGACTACCCCAAAA | gaccactgat | GATTTAGACTAC | Ebola | 1b |
| | _guide_22 | | ACGAAGGGGACTAAAACgac | gtttttggctc | CCCAAAAACGAA | ssRNA | |
| | | | cactgatgtttttggctccgtttga | cgtttga | GGGGACTAAAAC | | |
| | | | (SEQ ID. NO: 324) | (SEQ ID. | (SEQ ID. | | |
| | | | | NO: 325) | NO: 326) | | |
| 1b | Ebola_GP | LwaCas13a | GATTTAGACTACCCCAAAA | ctgaccactg | GATTTAGACTAC | Ebola | 1b |
| | _guide_23 | | ACGAAGGGGACTAAAACctg | atgtttttggc | CCCAAAAACGAA | ssRNA | |
| | | | accactgatgtttttggctccgttt | tccgttt | GGGGACTAAAAC | | |
| | | | (SEQ ID. NO: 327) | (SEQ ID. | (SEQ ID. | | |
| | | | | NO: 328) | NO: 329) | | |
| 1b | Ebola_GP | LwaCas13a | GATTTAGACTACCCCAAAA | ctctgaccac | GATTTAGACTAC | Ebola | 1b |
| | _guide_25 | | ACGAAGGGGACTAAAACctc | tgatgtttttg | CCCAAAAACGAA | ssRNA | |
| | | | tgaccactgatgtttttggctccgt | gctccgt | GGGGACTAAAAC | | |
| | | | (SEQ ID. NO: 330) | (SEQ ID. | (SEQ ID. | | |
| | | | | NO: 331) | NO: 332) | | |
| 1b | Ebola_GP | LwaCas13a | GATTTAGACTACCCCAAAA | cgccggact | GATTTAGACTAC | Ebola | 1b |
| | _guide_30 | | ACGAAGGGGACTAAAACcgc | ctgaccactg | CCCAAAAACGAA | ssRNA | |
| | | | cggactctgaccactgatgtttttg | atgtttttg | GGGGACTAAAAC | | |
| | | | (SEQ ID. NO: 333) | (SEQ ID. | (SEQ ID. | | |
| | | | | NO: 334) | NO: 335) | | |
| 1b | Ebola_GP | LwaCas13a | GATTTAGACTACCCCAAAA | cgcgccgga | GATTTAGACTAC | Ebola | 1b |
| | _guide_31 | | ACGAAGGGGACTAAAACcgc | ctctgaccac | CCCAAAAACGAA | ssRNA | |
| | | | gccggactctgaccactgatgtttt | tgatgtttt | GGGGACTAAAAC | | |
| | | | (SEQ ID. NO: 336) | (SEQ ID. | (SEQ ID. | | |
| | | | | NO: 337) | NO: 338) | | |
| 1b | Ebola_GP | LwaCas13a | GATTTAGACTACCCCAAAA | ttcgcgccg | GATTTAGACTAC | Ebola | 1b |
| | _guide_32 | | ACGAAGGGGACTAAAACttc | gactctgacc | CCCAAAAACGAA | ssRNA | |
| | | | gegccggactctgaccactgatgtt | actgatgtt | GGGGACTAAAAC | | |
| | | | (SEQ ID. NO: 339) | (SEQ ID. | (SEQ ID. | | |
| | | | | NO: 340) | NO: 341) | | |
| 1b | Ebola_GP | LwaCas13a | GATTTAGACTACCCCAAAA | gttcgcgcc | GATTTAGACTAC | Ebola | 1b |
| | _guide_33 | | ACGAAGGGGACTAAAACgtt | ggactctga | CCCAAAAACGAA | ssRNA | |
| | | | cgcgccggactctgaccactgatgt | ccactgatgt | GGGGACTAAAAC | | |
| | | | (SEQ ID. NO: 342) | (SEQ ID. | (SEQ ID. | | |
| | | | | NO: 343) | NO: 344) | | |
| 1b | Ebola_GP | LwaCas13a | GATTTAGACTACCCCAAAA | agttcgcgc | GATTTAGACTAC | Ebola | 1b |
| | _guide_34 | | ACGAAGGGGACTAAAACagt | cggactctg | CCCAAAAACGAA | ssRNA | |
| | | | tcgcgccggactctgaccactgatg | accactgatg | GGGGACTAAAAC | | |
| | | | (SEQ ID. NO: 345) | (SEQ ID. | (SEQ ID. | | |
| | | | | NO: 346) | NO: 347) | | |
| 1b | Ebola_GP | LwaCas13a | GATTTAGACTACCCCAAAA | aagttcgcg | GATTTAGACTAC | Ebola | 1b |
| | _guide_35 | | ACGAAGGGGACTAAAACaag | ccggactct | CCCAAAAACGAA | ssRNA | |
| | | | ttcgcgccggactctgaccactgat | gaccactgat | GGGGACTAAAAC | | |
| | | | (SEQ ID. NO: 348) | (SEQ ID. | (SEQ ID. | | |
| | | | | NO: 349) | NO: 350) | | |
| 1b | Ebola_GP | LwaCas13a | GATTTAGACTACCCCAAAA | agaagttcg | GATTTAGACTAC | Ebola | 1b |
| | _guide_36 | | ACGAAGGGGACTAAAACaga | cgccggact | CCCAAAAACGAA | ssRNA | |
| | | | agttcgcgccggactctgaccactg | ctgaccactg | GGGGACTAAAAC | | |
| | | | (SEQ ID. NO: 351) | (SEQ ID. | (SEQ ID. | | |
| | | | | NO: 352) | NO: 353) | | |
| 1b | Ebola_GP | LwaCas13a | GATTTAGACTACCCCAAAA | ggtcggaag | GATTTAGACTAC | Ebola | 1b |
| | _guide_42 | | ACGAAGGGGACTAAAACggt | aagttcgcg | CCCAAAAACGAA | ssRNA | |
| | | | cggaagaagttcgcgccggactctg | ccggactct | GGGGACTAAAAC | | |
| | | | (SEQ ID. NO: 354) | g (SEQ | (SEQ ID. | | |
| | | | | ID. NO: | NO: 356) | | |
| | | | | 355) | | | |
| 1b | Ebola_GP | LwaCas13a | GATTTAGACTACCCCAAAA | ctgggtcgg | GATTTAGACTAC | Ebola | 1b |
| | _guide_43 | | ACGAAGGGGACTAAAACctg | aagaagttc | CCCAAAAACGAA | ssRNA | |
| | | | ggtcggaagaagttcgcgccggact | gcgccggac | GGGGACTAAAAC | | |
| | | | (SEQ ID. NO: 357) | t (SEQ | (SEQ ID. | | |
| | | | | ID. NO: | NO: 359) | | |
| | | | | 358) | | | |
| 1b | Ebola_GP | LwaCas13a | GATTTAGACTACCCCAAAA | cctgggtcg | GATTTAGACTAC | Ebola | 1b |
| | _guide_44 | | ACGAAGGGGACTAAAACcct | gaagaagtt | CCCAAAAACGAA | ssRNA | |
| | | | gggtcggaagaagttcgcgccggac | cgcgccgga | GGGGACTAAAAC | | |
| | | | (SEQ ID. NO: 360) | c (SEQ | (SEQ ID. | | |
| | | | | ID. NO: | NO: 362) | | |
| | | | | 361) | | | |
| 1b | Ebola_GP | LwaCas13a | GATTTAGACTACCCCAAAA | ccctgggtc | GATTTAGACTAC | Ebola | 1b |
| | _guide_45 | | ACGAAGGGGACTAAAACccc | ggaagaagt | CCCAAAAACGAA | ssRNA | |
| | | | tgggtcggaagaagttcgcgccgga | tcgcgccgg | GGGGACTAAAAC | | |
| | | | (SEQ ID. NO: 363) | a (SEQ | (SEQ ID. | | |
| | | | | ID. NO: | NO: 365) | | |
| | | | | 364) | | | |
| 1b | Ebola_GP | LwaCas13a | GATTTAGACTACCCCAAAA | tccctgggtc | GATTTAGACTAC | Ebola | 1b |
| | _guide_46 | | ACGAAGGGGACTAAAACtcc | ggaagaagt | CCCAAAAACGAA | ssRNA | |
| | | | ctgggtcggaagaagttcgcgccgg | tcgcgccgg | GGGGACTAAAAC | | |
| | | | (SEQ ID. NO: 366) | (SEQ ID. | (SEQ ID. | | |
| | | | | NO: 367) | NO: 368) | | |
| 1b | Ebola_GP | LwaCas13a | GATTTAGACTACCCCAAAA | ggtccctgg | GATTTAGACTAC | Ebola | 1b |
| | _guide_48 | | ACGAAGGGGACTAAAACggt | gtcggaaga | CCCAAAAACGAA | ssRNA | |
| | | | ccctgggtcggaagaagttcgcgcc | agttcgcgc | GGGGACTAAAAC | | |
| | | | (SEQ ID. NO: 369) | c (SEQ | (SEQ ID. | | |
| | | | | ID. NO: | NO: 371) | | |
| | | | | 370) | | | |
| 1b | Ebola_GP | LwaCas13a | GATTTAGACTACCCCAAAA | gttggtccct | GATTTAGACTAC | Ebola | 1b |
| | _guide_49 | | ACGAAGGGGACTAAAACgtt | gggtcggaa | CCCAAAAACGAA | ssRNA | |
| | | | ggtccctgggtcggaagaagttcgc | gaagttcgc | GGGGACTAAAAC | | |
| | | | (SEQ ID. NO: 372) | (SEQ ID. | (SEQ ID. | | |
| | | | | NO: 373) | NO: 374) | | |
| 1b | Ebola_GP | LwaCas13a | GATTTAGACTACCCCAAAA | agttgttgtgt | GATTTAGACTAC | Ebola | 1b |
| | _guide_55 | | ACGAAGGGGACTAAAACagt | tggtccctgg | CCCAAAAACGAA | ssRNA | |
| | | | tgttgtgttggtccctgggtcggaa | gtcggaa | GGGGACTAAAAC | | |
| | | | (SEQ ID. NO: 375) | (SEQ ID. | (SEQ ID. | | |
| | | | | NO: 376) | NO: 377) | | |
| 1b | Ebola_GP | LwaCas13a | GATTTAGACTACCCCAAAA | tcagttgttgt | GATTTAGACTAC | Ebola | 1b |
| | _guide_57 | | ACGAAGGGGACTAAAACtca | gttggtccct | CCCAAAAACGAA | ssRNA | |
| | | | gttgttgtgttggtccctgggtcgg | gggtcgg | GGGGACTAAAAC | | |
| | | | (SEQ ID. NO: 378) | (SEQ ID. | (SEQ ID. | | |
| | | | | NO: 379) | NO: 380) | | |
| 1b | Ebola_GP | LwaCas13a | GATTTAGACTACCCCAAAA | ttcagttgttg | GATTTAGACTAC | Ebola | 1b |
| | _guide_58 | | ACGAAGGGGACTAAAACttc | tgttggtccct | CCCAAAAACGAA | ssRNA | |
| | | | agttgttgtgttggtccctgggtcg | gggtcg | GGGGACTAAAAC | | |
| | | | (SEQ ID. NO: 381) | (SEQ ID. | (SEQ ID. | | |
| | | | | NO: 382) | NO: 383) | | |
| 1b | Ebola_GP | LwaCas13a | GATTTAGACTACCCCAAAA | cttcagttgtt | GATTTAGACTAC | Ebola | 1b |
| | _guide_59 | | ACGAAGGGGACTAAAACctt | gtgttggtcc | CCCAAAAACGAA | ssRNA | |
| | | | cagttgttgtgttggtccctgggtc | ctgggtc | GGGGACTAAAAC | | |
| | | | (SEQ ID. NO: 384) | (SEQ ID. | (SEQ ID. | | |
| | | | | NO: 385) | NO: 386) | | |
| 1b | Ebola_GP | LwaCas13a | GATTTAGACTACCCCAAAA | tcttcagttgt | GATTTAGACTAC | Ebola | 1b |
| | _guide_60 | | ACGAAGGGGACTAAAACtct | tgtgttggtc | CCCAAAAACGAA | ssRNA | |
| | | | tcagttgttgtgttggtccctgggt | cctgggt | GGGGACTAAAAC | | |
| | | | (SEQ ID. NO: 387) | (SEQ ID. | (SEQ ID. | | |
| | | | | NO: 388) | NO: 389) | | |
| 1b | Ebola_GP | LwaCas13a | GATTTAGACTACCCCAAAA | tggtcttcagt | GATTTAGACTAC | Ebola | 1b |
| | _guide_61 | | ACGAAGGGGACTAAAACtgg | tgttgtgttgg | CCCAAAAACGAA | ssRNA | |
| | | | tcttcagttgttgtgttggtccctg | tccctg | GGGGACTAAAAC | | |
| | | | (SEQ ID. NO: 390) | (SEQ ID. | (SEQ ID. | | |
| | | | | NO: 391) | NO: 392) | | |
| 1b | Ebola_GP | LwaCas13a | GATTTAGACTACCCCAAAA | attttgtggtc | GATTTAGACTAC | Ebola | 1b |
| | _guide_66 | | ACGAAGGGGACTAAAACatt | ttcagttgttg | CCCAAAAACGAA | ssRNA | |
| | | | ttgtggtcttcagttgttgtgttgg | tgttgg | GGGGACTAAAAC | | |
| | | | (SEQ ID. NO: 393) | (SEQ ID. | (SEQ ID. | | |
| | | | | NO: 394) | NO: 395) | | |
| 1b | Ebola_GP | LwaCas13a | GATTTAGACTACCCCAAAA | gccatgatttt | GATTTAGACTAC | Ebola | 1b |
| | _guide_70 | | ACGAAGGGGACTAAAACgcc | gtggtcttca | CCCAAAAACGAA | ssRNA | |
| | | | atgattttgtggtcttcagttgttg | gttgttg | GGGGACTAAAAC | | |
| | | | (SEQ ID. NO: 396) | (SEQ ID. | (SEQ ID. | | |
| | | | | NO: 397) | NO: 398) | | |
| 1b | Ebola_GP | LwaCas13a | GATTTAGACTACCCCAAAA | aagccatgat | GATTTAGACTAC | Ebola | 1b |
| | _guide_72 | | ACGAAGGGGACTAAAACaag | tttgtggtctt | CCCAAAAACGAA | ssRNA | |
| | | | ccatgattttgtggtcttcagttgt | cagttgt | GGGGACTAAAAC | | |
| | | | (SEQ ID. NO: 399) | (SEQ ID. | (SEQ ID. | | |
| | | | | NO: 400) | NO: 401) | | |
| 1b | Ebola_GP | LwaCas13a | GATTTAGACTACCCCAAAA | ctgaagccat | GATTTAGACTAC | Ebola | 1b |
| | _guide_73 | | ACGAAGGGGACTAAAACctg | gattttgtggt | CCCAAAAACGAA | ssRNA | |
| | | | aagccatgattttgtggtcttcagt | cttcagt | GGGGACTAAAAC | | |
| | | | (SEQ ID. NO: 402) | (SEQ ID. | (SEQ ID. | | |
| | | | | NO: 403) | NO: 404) | | |
| 1b | Ebola_GP | LwaCas13a | GATTTAGACTACCCCAAAA | gaattttctga | GATTTAGACTAC | Ebola | 1b |
| | _guide_78 | | ACGAAGGGGACTAAAACgaa | agccatgatt | CCCAAAAACGAA | ssRNA | |
| | | | ttttctgaagccatgattttgtggt | ttgtggt | GGGGACTAAAAC | | |
| | | | (SEQ ID. NO: 405) | (SEQ ID. | (SEQ ID. | | |
| | | | | NO: 406) | NO: 407) | | |
| 1b | Ebola_GP | LwaCas13a | GATTTAGACTACCCCAAAA | agaggaattt | GATTTAGACTAC | Ebola | 1b |
| | _guide_81 | | ACGAAGGGGACTAAAACaga | tctgaagcca | CCCAAAAACGAA | ssRNA | |
| | | | ggaattttctgaagccatgattttg | tgattttg | GGGGACTAAAAC | | |
| | | | (SEQ ID. NO: 408) | (SEQ ID. | (SEQ ID. | | |
| | | | | NO: 409) | NO: 410) | | |
| 1b | Ebola_GP | LwaCas13a | GATTTAGACTACCCCAAAA | cagaggaat | GATTTAGACTAC | Ebola | 1b |
| | _guide_82 | | ACGAAGGGGACTAAAACcag | tttctgaagc | CCCAAAAACGAA | ssRNA | |
| | | | aggaattttctgaagccatgatttt | catgatttt | GGGGACTAAAAC | | |
| | | | (SEQ ID. NO: 411) | (SEQ ID. | (SEQ ID. | | |
| | | | | NO: 412) | NO: 413) | | |
| 1b | Ebola_GP | LwaCas13a | GATTTAGACTACCCCAAAA | cattgcaga | GATTTAGACTAC | Ebola | 1b |
| | _guide_85 | | ACGAAGGGGACTAAAACcat | ggaattttctg | CCCAAAAACGAA | ssRNA | |
| | | | tgcagaggaattttctgaagccatg | aagccatg | GGGGACTAAAAC | | |
| | | | (SEQ ID. NO: 414) | (SEQ ID. | (SEQ ID. | | |
| | | | | NO: 415) | NO: 416) | | |
| 1b | Ebola_GP | LwaCas13a | GATTTAGACTACCCCAAAA | cacttgaacc | GATTTAGACTAC | Ebola | 1b |
| | _guide_90 | | ACGAAGGGGACTAAAACcac | attgcagag | CCCAAAAACGAA | ssRNA | |
| | | | ttgaaccattgcagaggaattttct | gaattttct | GGGGACTAAAAC | | |
| | | | (SEQ ID. NO: 417) | (SEQ ID. | (SEQ ID. | | |
| | | | | NO: 418) | NO: 419) | | |
| 7a | ssRNA1_ | LwaCas13a | GATTTAGACTACCCCAAAA | ccccgggta | GATTTAGACTAC | ssRNA1 | 7a |
| | guide_01 | | ACGAAGGGGACTAAAACccc | ccgagctcg | CCCAAAAACGAA | | |
| | | | cgggtaccgagctcgaattcactgg | aattcactgg | GGGGACTAAAAC | | |
| | | | (SEQ ID. NO: 420) | (SEQ ID. | (SEQ ID. | | |
| | | | | NO: 421) | NO: 422) | | |
| 7a | ssRNA1_ | LwaCas13a | GATTTAGACTACCCCAAAA | tccccgggt | GATTTAGACTAC | ssRNA1 | 7a |
| | guide_02 | | ACGAAGGGGACTAAAACtcc | accgagctc | CCCAAAAACGAA | | |
| | | | ccgggtaccgagctcgaattcactg | gaattcactg | GGGGACTAAAAC | | |
| | | | (SEQ ID. NO: 423) | (SEQ ID. | (SEQ ID. | | |
| | | | | NO: 424) | NO: 425) | | |
| 7a | ssRNA1_ | LwaCas13a | GATTTAGACTACCCCAAAA | atccccggg | GATTTAGACTAC | ssRNA1 | 7a |
| | guide_03 | | ACGAAGGGGACTAAAACatc | taccgagctc | CCCAAAAACGAA | | |
| | | | cccgggtaccgagctcgaattcact | gaattcact | GGGGACTAAAAC | | |
| | | | (SEQ ID. NO: 426) | (SEQ ID. | (SEQ ID. | | |
| | | | | NO: 427) | NO: 428) | | |
| 7a | ssRNA1_ | LwaCas13a | GATTTAGACTACCCCAAAA | aggatcccc | GATTTAGACTAC | ssRNA1 | 7a |
| | guide_04 | | ACGAAGGGGACTAAAACagg | gggtaccga | CCCAAAAACGAA | | |
| | | | atccccgggtaccgagctcgaattc | gctcgaattc | GGGGACTAAAAC | | |
| | | | (SEQ ID. NO: 429) | (SEQ ID. | (SEQ ID. | | |
| | | | | NO: 430) | NO: 431) | | |
| 7a | ssRNA1_ | LwaCas13a | GATTTAGACTACCCCAAAA | agaggatcc | GATTTAGACTAC | ssRNA1 | 7a |
| | guide_05 | | ACGAAGGGGACTAAAACaga | ccgggtacc | CCCAAAAACGAA | | |
| | | | ggatccccgggtaccgagctcgaat | gagctcgaa | GGGGACTAAAAC | | |
| | | | (SEQ ID. NO: 432) | t (SEQ | (SEQ ID. | | |
| | | | | ID. NO: | NO: 434) | | |
| | | | | 433) | | | |
| 7a | ssRNA1_ | LwaCas13a | GATTTAGACTACCCCAAAA | tctagaggat | GATTTAGACTAC | ssRNA1 | 7a |
| | guide_06 | | ACGAAGGGGACTAAAACtct | ccccgggta | CCCAAAAACGAA | | |
| | | | agaggatccccgggtaccgagctcg | ccgagctcg | GGGGACTAAAAC | | |
| | | | (SEQ ID. NO: 435) | (SEQ ID. | (SEQ ID. | | |
| | | | | NO: 436) | NO: 437) | | |
| 7a | ssRNA1_ | LwaCas13a | GATTTAGACTACCCCAAAA | ttctagagga | GATTTAGACTAC | ssRNA1 | 7a |
| | guide_07 | | ACGAAGGGGACTAAAACttc | tccccgggt | CCCAAAAACGAA | | |
| | | | tagaggatccccgggtaccgagctc | accgagctc | GGGGACTAAAAC | | |
| | | | (SEQ ID. NO: 438) | (SEQ ID. | (SEQ ID. | | |
| | | | | NO: 439) | NO: 440) | | |
| 7a | ssRNA1_ | LwaCas13a | GATTTAGACTACCCCAAAA | atttctagag | GATTTAGACTAC | ssRNA1 | 7a |
| | guide_08 | | ACGAAGGGGACTAAAACatt | gatccccgg | CCCAAAAACGAA | | |
| | | | tctagaggatccccgggtaccgagc | gtaccgagc | GGGGACTAAAAC | | |
| | | | (SEQ ID. NO: 441) | (SEQ ID. | (SEQ ID. | | |
| | | | | NO: 442) | NO: 443) | | |
| 7a | ssRNA1_ | LwaCas13a | GATTTAGACTACCCCAAAA | tatttctagag | GATTTAGACTAC | ssRNA1 | 7a |
| | guide_09 | | ACGAAGGGGACTAAAACtat | gatccccgg | CCCAAAAACGAA | | |
| | | | ttctagaggatccccgggtaccgag | gtaccgag | GGGGACTAAAAC | | |
| | | | (SEQ ID. NO: 444) | (SEQ ID. | (SEQ ID. | | |
| | | | | NO: 445) | NO: 446) | | |
| 7a | ssRNA1_ | LwaCas13a | GATTTAGACTACCCCAAAA | ccatatttcta | GATTTAGACTAC | ssRNA1 | 7a |
| | guide_10 | | ACGAAGGGGACTAAAACcca | gaggatccc | CCCAAAAACGAA | | |
| | | | tatttctagaggatccccgggtacc | cgggtacc | GGGGACTAAAAC | | |
| | | | (SEQ ID. NO: 447) | (SEQ ID. | (SEQ ID. | | |
| | | | | NO: 448) | NO: 449) | | |
| 7a | ssRNA1_ | LwaCas13a | GATTTAGACTACCCCAAAA | tccatatttct | GATTTAGACTAC | ssRNA1 | 7a |
| | guide_11 | | ACGAAGGGGACTAAAACtcc | agaggatcc | CCCAAAAACGAA | | |
| | | | atatttctagaggatccccgggtac | ccgggtac | GGGGACTAAAAC | | |
| | | | (SEQ ID. NO: 450) | (SEQ ID. | (SEQ ID. | | |
| | | | | NO: 451) | NO: 452) | | |
| 7a | ssRNA1_ | LwaCas13a | GATTTAGACTACCCCAAAA | atccatatttc | GATTTAGACTAC | ssRNA1 | 7a |
| | guide_12 | | ACGAAGGGGACTAAAACatc | tagaggatc | CCCAAAAACGAA | | |
| | | | catatttctagaggatccccgggta | cccgggta | GGGGACTAAAAC | | |
| | | | (SEQ ID. NO: 453) | (SEQ ID. | (SEQ ID. | | |
| | | | | NO: 454) | NO: 455) | | |
| 7a | ssRNA1_ | LwaCas13a | GATTTAGACTACCCCAAAA | taatccatatt | GATTTAGACTAC | ssRNA1 | 7a |
| | guide_13 | | ACGAAGGGGACTAAAACtaa | tctagaggat | CCCAAAAACGAA | | |
| | | | tccatatttctagaggatccccggg | ccccggg | GGGGACTAAAAC | | |
| | | | (SEQ ID. NO: 456) | (SEQ ID. | (SEQ ID. | | |
| | | | | NO: 457) | NO: 458) | | |
| 7a | ssRNA1_ | LwaCas13a | GATTTAGACTACCCCAAAA | gtaatccata | GATTTAGACTAC | ssRNA1 | 7a |
| | guide_14 | | ACGAAGGGGACTAAAACgta | tttctagagg | CCCAAAAACGAA | | |
| | | | atccatatttctagaggatccccgg | atccccgg | GGGGACTAAAAC | | |
| | | | (SEQ ID. NO: 459) | (SEQ ID. | (SEQ ID. | | |
| | | | | NO: 460) | NO: 461) | | |
| 7a | ssRNA1_ | LwaCas13a | GATTTAGACTACCCCAAAA | taccaagtaa | GATTTAGACTAC | ssRNA1 | 7a |
| | guide_15 | | ACGAAGGGGACTAAAACtac | tccatatttct | CCCAAAAACGAA | | |
| | | | caagtaatccatatttctagaggat | agaggat | GGGGACTAAAAC | | |
| | | | (SEQ ID. NO: 462) | (SEQ ID. | (SEQ ID. | | |
| | | | | NO: 463) | NO: 464) | | |
| 7a | ssRNA1_ | LwaCas13a | GATTTAGACTACCCCAAAA | tctaccaagt | GATTTAGACTAC | ssRNA1 | 7a |
| | guide_16 | | ACGAAGGGGACTAAAACtct | aatccatattt | CCCAAAAACGAA | | |
| | | | accaagtaatccatatttctagagg | ctagagg | GGGGACTAAAAC | | |
| | | | (SEQ ID. NO: 465) | (SEQ ID. | (SEQ ID. | | |
| | | | | NO: 466) | NO: 467) | | |
| 7a | ssRNA1_ | LwaCas13a | GATTTAGACTACCCCAAAA | gttctaccaa | GATTTAGACTAC | ssRNA1 | 7a |
| | guide_17 | | ACGAAGGGGACTAAAACgtt | gtaatccata | CCCAAAAACGAA | | |
| | | | ctaccaagtaatccatatttctaga | tttctaga | GGGGACTAAAAC | | |
| | | | (SEQ ID. NO: 468) | (SEQ ID. | (SEQ ID. | | |
| | | | | NO: 469) | NO: 470) | | |
| 7a | ssRNA1_ | LwaCas13a | GATTTAGACTACCCCAAAA | gctgttctac | GATTTAGACTAC | ssRNA1 | 7a |
| | guide_18 | | ACGAAGGGGACTAAAACgct | caagtaatcc | CCCAAAAACGAA | | |
| | | | gttctaccaagtaatccatatttct | atatttct | GGGGACTAAAAC | | |
| | | | (SEQ ID. NO: 471) | (SEQ ID. | (SEQ ID. | | |
| | | | | NO: 472) | NO: 473) | | |
| 7a | ssRNA1_ | LwaCas13a | GATTTAGACTACCCCAAAA | attgctgttct | GATTTAGACTAC | ssRNA1 | 7a |
| | guide_20 | | ACGAAGGGGACTAAAACatt | accaagtaat | CCCAAAAACGAA | | |
| | | | gctgttctaccaagtaatccatatt | ccatatt | GGGGACTAAAAC | | |
| | | | (SEQ ID. NO: 474) | (SEQ ID. | (SEQ ID. | | |
| | | | | NO: 475) | NO: 476) | | |
| 7a | ssRNA1_ | LwaCas13a | GATTTAGACTACCCCAAAA | tagattgctgt | GATTTAGACTAC | ssRNA1 | 7a |
| | guide_21 | | ACGAAGGGGACTAAAACtag | tctaccaagt | CCCAAAAACGAA | | |
| | | | attgctgttctaccaagtaatccat | aatccat | GGGGACTAAAAC | | |
| | | | (SEQ ID. NO: 477) | (SEQ ID. | (SEQ ID. | | |
| | | | | NO: 478) | NO: 479) | | |
| 7a | ssRNA1_ | LwaCas13a | GATTTAGACTACCCCAAAA | gtagattgct | GATTTAGACTAC | ssRNA1 | 7a |
| | guide_22 | | ACGAAGGGGACTAAAACgta | gttctaccaa | CCCAAAAACGAA | | |
| | | | gattgctgttctaccaagtaatcca | gtaatcca | GGGGACTAAAAC | | |
| | | | (SEQ ID. NO: 480) | (SEQ ID. | (SEQ ID. | | |
| | | | | NO: 481) | NO: 482) | | |
| 7a | ssRNA1_ | LwaCas13a | GATTTAGACTACCCCAAAA | agtagattgc | GATTTAGACTAC | ssRNA1 | 7a |
| | guide_23 | | ACGAAGGGGACTAAAACagt | tgttctacca | CCCAAAAACGAA | | |
| | | | agattgctgttctaccaagtaatcc | agtaatcc | GGGGACTAAAAC | | |
| | | | (SEQ ID. NO: 483) | (SEQ ID. | (SEQ ID. | | |
| | | | | NO: 484) | NO: 485) | | |
| 7a | ssRNA1_ | LwaCas13a | GATTTAGACTACCCCAAAA | gagtagattg | GATTTAGACTAC | ssRNA1 | 7a |
| | guide_24 | | ACGAAGGGGACTAAAACgag | ctgttctacc | CCCAAAAACGAA | | |
| | | | tagattgctgttctaccaagtaatc | aagtaatc | GGGGACTAAAAC | | |
| | | | (SEQ ID. NO: 486) | (SEQ ID. | (SEQ ID. | | |
| | | | | NO: 487) | NO: 488) | | |
| 7a | ssRNA1_ | LwaCas13a | GATTTAGACTACCCCAAAA | tcgagtagat | GATTTAGACTAC | ssRNA1 | 7a |
| | guide_25 | | ACGAAGGGGACTAAAACtcg | tgctgttctac | CCCAAAAACGAA | | |
| | | | agtagattgctgttctaccaagtaa | caagtaa | GGGGACTAAAAC | | |
| | | | (SEQ ID. NO: 489) | (SEQ ID. | (SEQ ID. | | |
| | | | | NO: 490) | NO: 491) | | |
| 7a | ssRNA1_ | LwaCas13a | GATTTAGACTACCCCAAAA | gtcgagtag | GATTTAGACTAC | ssRNA1 | 7a |
| | guide_26 | | ACGAAGGGGACTAAAACgtc | attgctgttct | CCCAAAAACGAA | | |
| | | | gagtagattgctgttctaccaagta | accaagta | GGGGACTAAAAC | | |
| | | | (SEQ ID. NO: 492) | (SEQ ID. | (SEQ ID. | | |
| | | | | NO: 493) | NO: 494) | | |
| 7a | ssRNA1_ | LwaCas13a | GATTTAGACTACCCCAAAA | caggtcgag | GATTTAGACTAC | ssRNA1 | 7a |
| | guide_28 | | ACGAAGGGGACTAAAACcag | tagattgctgt | CCCAAAAACGAA | | |
| | | | gtcgagtagattgctgttctaccaa | tctaccaa | GGGGACTAAAAC | | |
| | | | (SEQ ID. NO: 495) | (SEQ ID. | (SEQ ID. | | |
| | | | | NO: 496) | NO: 497) | | |
| 7a | ssRNA1_ | LwaCas13a | GATTTAGACTACCCCAAAA | gcaggtcga | GATTTAGACTAC | ssRNA1 | 7a |
| | guide_29 | | ACGAAGGGGACTAAAACgca | gtagattgct | CCCAAAAACGAA | | |
| | | | ggtcgagtagattgctgttctacca | gttctacca | GGGGACTAAAAC | | |
| | | | (SEQ ID. NO: 498) | (SEQ ID. | (SEQ ID. | | |
| | | | | NO: 499) | NO: 500) | | |
| 7a | ssRNA1_ | LwaCas13a | GATTTAGACTACCCCAAAA | tgcaggtcg | GATTTAGACTAC | ssRNA1 | 7a |
| | guide_30 | | ACGAAGGGGACTAAAACtgc | agtagattgc | CCCAAAAACGAA | | |
| | | | aggtcgagtagattgctgttctacc | tgttctacc | GGGGACTAAAAC | | |
| | | | (SEQ ID. NO: 501) | (SEQ ID. | (SEQ ID. | | |
| | | | | NO: 502) | NO: 503) | | |
| 7a | ssRNA1_ | LwaCas13a | GATTTAGACTACCCCAAAA | ctgcaggtc | GATTTAGACTAC | ssRNA1 | 7a |
| | guide_31 | | ACGAAGGGGACTAAAACctg | gagtagattg | CCCAAAAACGAA | | |
| | | | caggtcgagtagattgctgttctac | ctgttctac | GGGGACTAAAAC | | |
| | | | (SEQ ID. NO: 504) | (SEQ ID. | (SEQ ID. | | |
| | | | | NO: 505) | NO: 506) | | |
| 7a | ssRNA1_ | LwaCas13a | GATTTAGACTACCCCAAAA | cctgcaggt | GATTTAGACTAC | ssRNA1 | 7a |
| | guide_32 | | ACGAAGGGGACTAAAACcct | cgagtagatt | CCCAAAAACGAA | | |
| | | | gcaggtcgagtagattgctgttcta | gctgttcta | GGGGACTAAAAC | | |
| | | | (SEQ ID. NO: 507) | (SEQ ID. | (SEQ ID. | | |
| | | | | NO: 508) | NO: 509) | | |
| 7a | ssRNA1_ | LwaCas13a | GATTTAGACTACCCCAAAA | tgcctgcag | GATTTAGACTAC | ssRNA1 | 7a |
| | guide_33 | | ACGAAGGGGACTAAAACtgc | gtcgagtag | CCCAAAAACGAA | | |
| | | | ctgcaggtcgagtagattgctgttc | attgctgttc | GGGGACTAAAAC | | |
| | | | (SEQ ID. NO: 510) | (SEQ ID. | (SEQ ID. | | |
| | | | | NO: 511) | NO: 512) | | |
| 7a | ssRNA1_ | LwaCas13a | GATTTAGACTACCCCAAAA | atgcctgca | GATTTAGACTAC | ssRNA1 | 7a |
| | guide_34 | | ACGAAGGGGACTAAAACatg | ggtcgagta | CCCAAAAACGAA | | |
| | | | cctgcaggtcgagtagattgctgtt | gattgctgtt | GGGGACTAAAAC | | |
| | | | (SEQ ID. NO: 513) | (SEQ ID. | (SEQ ID. | | |
| | | | | NO: 514) | NO: 515) | | |
| 7a | ssRNA1_ | LwaCas13a | GATTTAGACTACCCCAAAA | catgcctgca | GATTTAGACTAC | ssRNA1 | 7a |
| | guide_35 | | ACGAAGGGGACTAAAACcat | ggtcgagta | CCCAAAAACGAA | | |
| | | | gcctgcaggtcgagtagattgctgt | gattgctgt | GGGGACTAAAAC | | |
| | | | (SEQ ID. NO: 516) | (SEQ ID. | (SEQ ID. | | |
| | | | | NO: 517) | NO: 518) | | |
| 7a | ssRNA1_ | LwaCas13a | GATTTAGACTACCCCAAAA | tgcatgcctg | GATTTAGACTAC | ssRNA1 | 7a |
| | guide_36 | | ACGAAGGGGACTAAAACtgc | caggtcgag | CCCAAAAACGAA | | |
| | | | atgcctgcaggtcgagtagattgct | tagattgct | GGGGACTAAAAC | | |
| | | | (SEQ ID. NO: 519) | (SEQ ID. | (SEQ ID. | | |
| | | | | NO: 520) | NO: 521) | | |
| 7a | ssRNA1_ | LwaCas13a | GATTTAGACTACCCCAAAA | cttgcatgcc | GATTTAGACTAC | ssRNA1 | 7a |
| | guide_38 | | ACGAAGGGGACTAAAACctt | tgcaggtcg | CCCAAAAACGAA | | |
| | | | gcatgcctgcaggtcgagtagattg | agtagattg | GGGGACTAAAAC | | |
| | | | (SEQ ID. NO: 522) | (SEQ ID. | (SEQ ID. | | |
| | | | | NO: 523) | NO: 524) | | |
| 7a | ssRNA1_ | LwaCas13a | GATTTAGACTACCCCAAAA | agcttgcatg | GATTTAGACTAC | ssRNA1 | 7a |
| | guide_40 | | ACGAAGGGGACTAAAACagc | cctgcaggt | CCCAAAAACGAA | | |
| | | | ttgcatgcctgcaggtcgagtagat | cgagtagat | GGGGACTAAAAC | | |
| | | | (SEQ ID. NO: 525) | (SEQ ID. | (SEQ ID. | | |
| | | | | NO: 526) | NO: 527) | | |
| 7a | ssRNA1_ | LwaCas13a | GATTTAGACTACCCCAAAA | caagcttgca | GATTTAGACTAC | ssRNA1 | 7a |
| | guide_42 | | ACGAAGGGGACTAAAACcaa | tgcctgcag | CCCAAAAACGAA | | |
| | | | gcttgcatgcctgcaggtcgagtag | gtcgagtag | GGGGACTAAAAC | | |
| | | | (SEQ ID. NO: 528) | (SEQ ID. | (SEQ ID. | | |
| | | | | NO: 529) | NO: 530) | | |
| 7a | ssRNA1_ | LwaCas13a | GATTTAGACTACCCCAAAA | ccaagcttgc | GATTTAGACTAC | ssRNA1 | 7a |
| | guide_43 | | ACGAAGGGGACTAAAACcca | atgcctgca | CCCAAAAACGAA | | |
| | | | agcttgcatgcctgcaggtcgagta | ggtcgagta | GGGGACTAAAAC | | |
| | | | (SEQ ID. NO: 531) | (SEQ ID. | (SEQ ID. | | |
| | | | | NO: 532) | NO: 533) | | |
| 7a | ssRNA1_ | LwaCas13a | GATTTAGACTACCCCAAAA | cgccaagctt | GATTTAGACTAC | ssRNA1 | 7a |
| | guide_44 | | ACGAAGGGGACTAAAACcgc | gcatgcctg | CCCAAAAACGAA | | |
| | | | caagcttgcatgcctgcaggtcgag | caggtcgag | GGGGACTAAAAC | | |
| | | | (SEQ ID. NO: 534) | (SEQ ID. | (SEQ ID. | | |
| | | | | NO: 535) | NO: 536) | | |
| 7a | ssRNA1_ | LwaCas13a | GATTTAGACTACCCCAAAA | acgccaagc | GATTTAGACTAC | ssRNA1 | 7a |
| | guide_45 | | ACGAAGGGGACTAAAACacg | ttgcatgcct | CCCAAAAACGAA | | |
| | | | ccaagcttgcatgcctgcaggtcga | gcaggtcga | GGGGACTAAAAC | | |
| | | | (SEQ ID. NO: 537) | (SEQ ID. | (SEQ ID. | | |
| | | | | NO: 538) | NO: 539) | | |
| 7a | ssRNA1_ | LwaCas13a | GATTTAGACTACCCCAAAA | tacgccaag | GATTTAGACTAC | ssRNA1 | 7a |
| | guide_46 | | ACGAAGGGGACTAAAACtac | cttgcatgcc | CCCAAAAACGAA | | |
| | | | gccaagcttgcatgcctgcaggtcg | tgcaggtcg | GGGGACTAAAAC | | |
| | | | (SEQ ID. NO: 540) | (SEQ ID. | (SEQ ID. | | |
| | | | | NO: 541) | NO: 542) | | |
| 7a | ssRNA1_ | LwaCas13a | GATTTAGACTACCCCAAAA | ttacgccaag | GATTTAGACTAC | ssRNA1 | 7a |
| | guide_47 | | ACGAAGGGGACTAAAACtta | cttgcatgcc | CCCAAAAACGAA | | |
| | | | cgccaagcttgcatgcctgcaggtc | tgcaggtc | GGGGACTAAAAC | | |
| | | | (SEQ ID. NO: 543) | (SEQ ID. | (SEQ ID. | | |
| | | | | NO: 544) | NO: 545) | | |
| 7a | ssRNA1_ | LwaCas13a | GATTTAGACTACCCCAAAA | attacgccaa | GATTTAGACTAC | ssRNA1 | 7a |
| | guide_48 | | ACGAAGGGGACTAAAACatt | gcttgcatgc | CCCAAAAACGAA | | |
| | | | acgccaagcttgcatgcctgcaggt | ctgcaggt | GGGGACTAAAAC | | |
| | | | (SEQ ID. NO: 546) | (SEQ ID. | (SEQ ID. | | |
| | | | | NO: 547) | NO: 548) | | |
| 7a | ssRNA1_ | LwaCas13a | GATTTAGACTACCCCAAAA | gattacgcca | GATTTAGACTAC | ssRNA1 | 7a |
| | guide_49 | | ACGAAGGGGACTAAAACgat | agcttgcatg | CCCAAAAACGAA | | |
| | | | tacgccaagcttgcatgcctgcagg | cctgcagg | GGGGACTAAAAC | | |
| | | | (SEQ ID. NO: 549) | (SEQ ID. | (SEQ ID. | | |
| | | | | NO: 550) | NO: 551) | | |
| 7a | ssRNA1_ | LwaCas13a | GATTTAGACTACCCCAAAA | ccatgattac | GATTTAGACTAC | ssRNA1 | 7a |
| | guide_52 | | ACGAAGGGGACTAAAACcca | gccaagctt | CCCAAAAACGAA | | |
| | | | tgattacgccaagcttgcatgcctg | gcatgcctg | GGGGACTAAAAC | | |
| | | | (SEQ ID. NO: 552) | (SEQ ID. | (SEQ ID. | | |
| | | | | NO: 553) | NO: 554) | | |
| 7a | ssRNA1_ | LwaCas13a | GATTTAGACTACCCCAAAA | accatgatta | GATTTAGACTAC | ssRNA1 | 7a |
| | guide_53 | | ACGAAGGGGACTAAAACacc | cgccaagctt | CCCAAAAACGAA | | |
| | | | atgattacgccaagcttgcatgcct | gcatgcct | GGGGACTAAAAC | | |
| | | | (SEQ ID. NO: 555) | (SEQ ID. | (SEQ ID. | | |
| | | | | NO: 556) | NO: 557) | | |
| 7a | ssRNA1_ | LwaCas13a | GATTTAGACTACCCCAAAA | gaccatgatt | GATTTAGACTAC | ssRNA1 | 7a |
| | guide_54 | | ACGAAGGGGACTAAAACgac | acgccaagc | CCCAAAAACGAA | | |
| | | | catgattacgccaagcttgcatgcc | ttgcatgcc | GGGGACTAAAAC | | |
| | | | (SEQ ID. NO: 558) | (SEQ ID. | (SEQ ID. | | |
| | | | | NO: 559) | NO: 560) | | |
| 7a | ssRNA1_ | LwaCas13a | GATTTAGACTACCCCAAAA | atgaccatga | GATTTAGACTAC | ssRNA1 | 7a |
| | guide_55 | | ACGAAGGGGACTAAAACatg | ttacgccaag | CCCAAAAACGAA | | |
| | | | accatgattacgccaagcttgcatg | cttgcatg | GGGGACTAAAAC | | |
| | | | (SEQ ID. NO: 561) | (SEQ ID. | (SEQ ID. | | |
| | | | | NO: 562) | NO: 563) | | |
| 7a | ssRNA1_ | LwaCas13a | GATTTAGACTACCCCAAAA | tatgaccatg | GATTTAGACTAC | ssRNA1 | 7a |
| | guide_56 | | ACGAAGGGGACTAAAACtat | attacgccaa | CCCAAAAACGAA | | |
| | | | gaccatgattacgccaagcttgcat | gcttgcat | GGGGACTAAAAC | | |
| | | | (SEQ ID. NO: 564) | (SEQ ID. | (SEQ ID. | | |
| | | | | NO: 565) | NO: 566) | | |
| 7a | ssRNA1_ | LwaCas13a | GATTTAGACTACCCCAAAA | agctatgacc | GATTTAGACTAC | ssRNA1 | 7a |
| | guide_57 | | ACGAAGGGGACTAAAACagc | atgattacgc | CCCAAAAACGAA | | |
| | | | tatgaccatgattacgccaagcttg | caagcttg | GGGGACTAAAAC | | |
| | | | (SEQ ID. NO: 567) | (SEQ ID. | (SEQ ID. | | |
| | | | | NO: 568) | NO: 569) | | |
| 7a | ssRNA1_ | LwaCas13a | GATTTAGACTACCCCAAAA | cagctatgac | GATTTAGACTAC | ssRNA1 | 7a |
| | guide_58 | | ACGAAGGGGACTAAAACcag | catgattacg | CCCAAAAACGAA | | |
| | | | ctatgaccatgattacgccaagctt | ccaagctt | GGGGACTAAAAC | | |
| | | | (SEQ ID. NO: 570) | (SEQ ID. | (SEQ ID. | | |
| | | | | NO: 571) | NO: 572) | | |
| 7a | ssRNA1_ | LwaCas13a | GATTTAGACTACCCCAAAA | acagctatga | GATTTAGACTAC | ssRNA1 | 7a |
| | guide_59 | | ACGAAGGGGACTAAAACaca | ccatgattac | CCCAAAAACGAA | | |
| | | | gctatgaccatgattacgccaagct | gccaagct | GGGGACTAAAAC | | |
| | | | (SEQ ID. NO: 573) | (SEQ ID. | (SEQ ID. | | |
| | | | | NO: 574) | NO: 575) | | |
| 7a | ssRNA1_ | LwaCas13a | GATTTAGACTACCCCAAAA | aacagctatg | GATTTAGACTAC | ssRNA1 | 7a |
| | guide_60 | | ACGAAGGGGACTAAAACaac | accatgatta | CCCAAAAACGAA | | |
| | | | agctatgaccatgattacgccaagc | cgccaagc | GGGGACTAAAAC | | |
| | | | (SEQ ID. NO: 576) | (SEQ ID. | (SEQ ID. | | |
| | | | | NO: 577) | NO: 578) | | |
| 7a | ssRNA1_ | LwaCas13a | GATTTAGACTACCCCAAAA | aacacagga | GATTTAGACTAC | ssRNA1 | 7a |
| | guide_64 | | ACGAAGGGGACTAAAACaac | aacagctatg | CCCAAAAACGAA | | |
| | | | acaggaaacagctatgaccatgatt | accatgatt | GGGGACTAAAAC | | |
| | | | (SEQ ID. NO: 579) | (SEQ ID. | (SEQ ID. | | |
| | | | | NO: 580) | NO: 581) | | |
| 7a | ssRNA1_ | LwaCas13a | GATTTAGACTACCCCAAAA | taaacacag | GATTTAGACTAC | ssRNA1 | 7a |
| | guide_65 | | ACGAAGGGGACTAAAACtaa | gaaacagct | CCCAAAAACGAA | | |
| | | | acacaggaaacagctatgaccatga | atgaccatga | GGGGACTAAAAC | | |
| | | | (SEQ ID. NO: 582) | (SEQ ID. | (SEQ ID. | | |
| | | | | NO: 583) | NO: 584) | | |
| 7a | ssRNA1_ | LwaCas13a | GATTTAGACTACCCCAAAA | ataaacaca | GATTTAGACTAC | ssRNA1 | 7a |
| | guide_66 | | ACGAAGGGGACTAAAACata | ggaaacagc | CCCAAAAACGAA | | |
| | | | aacacaggaaacagctatgaccatg | tatgaccatg | GGGGACTAAAAC | | |
| | | | (SEQ ID. NO: 585) | (SEQ ID. | (SEQ ID. | | |
| | | | | NO: 586) | NO: 587) | | |
| 7a | ssRNA1_ | LwaCas13a | GATTTAGACTACCCCAAAA | gataaacac | GATTTAGACTAC | ssRNA1 | 7a |
| | guide_67 | | ACGAAGGGGACTAAAACgat | aggaaacag | CCCAAAAACGAA | | |
| | | | aaacacaggaaacagctatgaccat | ctatgaccat | GGGGACTAAAAC | | |
| | | | (SEQ ID. NO: 588) | (SEQ ID. | (SEQ ID. | | |
| | | | | NO: 589) | NO: 590) | | |
| 7a | ssRNA1_ | LwaCas13a | GATTTAGACTACCCCAAAA | ggataaaca | GATTTAGACTAC | ssRNA1 | 7a |
| | guide_68 | | ACGAAGGGGACTAAAACgga | caggaaaca | CCCAAAAACGAA | | |
| | | | taaacacaggaaacagctatgacca | gctatgacca | GGGGACTAAAAC | | |
| | | | (SEQ ID. NO: 591) | (SEQ ID. | (SEQ ID. | | |
| | | | | NO: 592) | NO: 593) | | |
| 7a | ssRNA1_ | LwaCas13a | GATTTAGACTACCCCAAAA | cggataaac | GATTTAGACTAC | ssRNA1 | 7a |
| | guide_69 | | ACGAAGGGGACTAAAACcgg | acaggaaac | CCCAAAAACGAA | | |
| | | | ataaacacaggaaacagctatgacc | agctatgacc | GGGGACTAAAAC | | |
| | | | (SEQ ID. NO: 594) | (SEQ ID. | (SEQ ID. | | |
| | | | | NO: 595) | NO: 596) | | |
| 7a | ssRNA1_ | LwaCas13a | GATTTAGACTACCCCAAAA | gcggataaa | GATTTAGACTAC | ssRNA1 | 7a |
| | guide_70 | | ACGAAGGGGACTAAAACgcg | cacaggaaa | CCCAAAAACGAA | | |
| | | | gataaacacaggaaacagctatgac | cagctatgac | GGGGACTAAAAC | | |
| | | | (SEQ ID. NO: 597) | (SEQ ID. | (SEQ ID. | | |
| | | | | NO: 598) | NO: 599) | | |
| 7a | ssRNA1_ | LwaCas13a | GATTTAGACTACCCCAAAA | agcggataa | GATTTAGACTAC | ssRNA1 | 7a |
| | guide_71 | | ACGAAGGGGACTAAAACagc | acacaggaa | CCCAAAAACGAA | | |
| | | | ggataaacacaggaaacagctatga | acagctatga | GGGGACTAAAAC | | |
| | | | (SEQ ID. NO: 600) | (SEQ ID. | (SEQ ID. | | |
| | | | | NO: 601) | NO: 602) | | |
| 7a | ssRNA1_ | LwaCas13a | GATTTAGACTACCCCAAAA | tgagcggat | GATTTAGACTAC | ssRNA1 | 7a |
| | guide_72 | | ACGAAGGGGACTAAAACtga | aaacacagg | CCCAAAAACGAA | | |
| | | | gcggataaacacaggaaacagctat | aaacagctat | GGGGACTAAAAC | | |
| | | | (SEQ ID. NO: 603) | (SEQ ID. | (SEQ ID. | | |
| | | | | NO: 604) | NO: 605) | | |
| 7a | ssRNA1_ | LwaCas13a | GATTTAGACTACCCCAAAA | tgtgagcgg | GATTTAGACTAC | ssRNA1 | 7a |
| | guide_73 | | ACGAAGGGGACTAAAACtgt | ataaacaca | CCCAAAAACGAA | | |
| | | | gagcggataaacacaggaaacagct | ggaaacagc | GGGGACTAAAAC | | |
| | | | (SEQ ID. NO: 606) | t (SEQ | (SEQ ID. | | |
| | | | | ID. NO: | NO: 608) | | |
| | | | | 607) | | | |
| 7a | ssRNA1_ | LwaCas13a | GATTTAGACTACCCCAAAA | ttgtgagcgg | GATTTAGACTAC | ssRNA1 | 7a |
| | guide_74 | | ACGAAGGGGACTAAAACttg | ataaacaca | CCCAAAAACGAA | | |
| | | | tgagcggataaacacaggaaacagc | ggaaacagc | GGGGACTAAAAC | | |
| | | | (SEQ ID. NO: 609) | (SEQ ID. | (SEQ ID. | | |
| | | | | NO: 610) | NO: 611) | | |
| 7a | ssRNA1_ | LwaCas13a | GATTTAGACTACCCCAAAA | ggaattgtga | GATTTAGACTAC | ssRNA1 | 7a |
| | guide_76 | | ACGAAGGGGACTAAAACgga | gcggataaa | CCCAAAAACGAA | | |
| | | | attgtgagcggataaacacaggaaa | cacaggaaa | GGGGACTAAAAC | | |
| | | | (SEQ ID. NO: 612) | (SEQ ID. | (SEQ ID. | | |
| | | | | NO: 613) | NO: 614) | | |
| 7a | ssRNA1_ | LwaCas13a | GATTTAGACTACCCCAAAA | tggaattgtg | GATTTAGACTAC | ssRNA1 | 7a |
| | guide_77 | | ACGAAGGGGACTAAAACtgg | agcggataa | CCCAAAAACGAA | | |
| | | | aattgtgagcggataaacacaggaa | acacaggaa | GGGGACTAAAAC | | |
| | | | (SEQ ID. NO: 615) | (SEQ ID. | (SEQ ID. | | |
| | | | | NO: 616) | NO: 617) | | |
| 7a | ssRNA1_ | LwaCas13a | GATTTAGACTACCCCAAAA | gtggaattgt | GATTTAGACTAC | ssRNA1 | 7a |
| | guide_78 | | ACGAAGGGGACTAAAACgtg | gagcggata | CCCAAAAACGAA | | |
| | | | gaattgtgagcggataaacacagga | aacacagga | GGGGACTAAAAC | | |
| | | | (SEQ ID. NO: 618) | (SEQ ID. | (SEQ ID. | | |
| | | | | NO: 619) | NO: 620) | | |
| 7a | ssRNA1_ | LwaCas13a | GATTTAGACTACCCCAAAA | tgtggaattg | GATTTAGACTAC | ssRNA1 | 7a |
| | guide_79 | | ACGAAGGGGACTAAAACtgt | tgagcggat | CCCAAAAACGAA | | |
| | | | ggaattgtgagcggataaacacagg | aaacacagg | GGGGACTAAAAC | | |
| | | | (SEQ ID. NO: 621) | (SEQ ID. | (SEQ ID. | | |
| | | | | NO: 622) | NO: 623) | | |
| 7a | ssRNA1_ | LwaCas13a | GATTTAGACTACCCCAAAA | tgtgtggaat | GATTTAGACTAC | ssRNA1 | 7a |
| | guide_80 | | ACGAAGGGGACTAAAACtgt | tgtgagcgg | CCCAAAAACGAA | | |
| | | | gtggaattgtgagcggataaacaca | ataaacaca | GGGGACTAAAAC | | |
| | | | (SEQ ID. NO: 624) | (SEQ ID. | (SEQ ID. | | |
| | | | | NO: 625) | NO: 626) | | |
| 7a | ssRNA1_ | LwaCas13a | GATTTAGACTACCCCAAAA | ttgtgtggaa | GATTTAGACTAC | ssRNA1 | 7a |
| | guide_81 | | ACGAAGGGGACTAAAACttg | ttgtgagcgg | CCCAAAAACGAA | | |
| | | | tgtggaattgtgagcggataaacac | ataaacac | GGGGACTAAAAC | | |
| | | | (SEQ ID. NO: 627) | (SEQ ID. | (SEQ ID. | | |
| | | | | NO: 628) | NO: 629) | | |
| 7a | ssRNA1_ | LwaCas13a | GATTTAGACTACCCCAAAA | gttgtgtgga | GATTTAGACTAC | ssRNA1 | 7a |
| | guide_82 | | ACGAAGGGGACTAAAACgtt | attgtgagcg | CCCAAAAACGAA | | |
| | | | gtgtggaattgtgagcggataaaca | gataaaca | GGGGACTAAAAC | | |
| | | | (SEQ ID. NO: 630) | (SEQ ID. | (SEQ ID. | | |
| | | | | NO: 631) | NO: 632) | | |
| 7a | ssRNA1_ | LwaCas13a | GATTTAGACTACCCCAAAA | atgttgtgtg | GATTTAGACTAC | ssRNA1 | 7a |
| | guide_83 | | ACGAAGGGGACTAAAACatg | gaattgtgag | CCCAAAAACGAA | | |
| | | | ttgtgtggaattgtgagcggataaa | cggataaa | GGGGACTAAAAC | | |
| | | | (SEQ ID. NO: 633) | (SEQ ID. | (SEQ ID. | | |
| | | | | NO: 634) | NO: 635) | | |
| 7a | ssRNA1_ | LwaCas13a | GATTTAGACTACCCCAAAA | tatgttgtgtg | GATTTAGACTAC | ssRNA1 | 7a |
| | guide_84 | | ACGAAGGGGACTAAAACtat | gaattgtgag | CCCAAAAACGAA | | |
| | | | gttgtgtggaattgtgagcggataa | cggataa | GGGGACTAAAAC | | |
| | | | (SEQ ID. NO: 636) | (SEQ ID. | (SEQ ID. | | |
| | | | | NO: 637) | NO: 638) | | |
| 7a | ssRNA1_ | LwaCas13a | GATTTAGACTACCCCAAAA | tegtatgttgt | GATTTAGACTAC | ssRNA1 | 7a |
| | guide_86 | | ACGAAGGGGACTAAAACtcg | gtggaattgt | CCCAAAAACGAA | | |
| | | | tatgttgtgtggaattgtgagcgga | gagcgga | GGGGACTAAAAC | | |
| | | | (SEQ ID. NO: 639) | (SEQ ID. | (SEQ ID. | | |
| | | | | NO: 640) | NO: 641) | | |
| 7a | ssRNA1_ | LwaCas13a | GATTTAGACTACCCCAAAA | ggctcgtatg | GATTTAGACTAC | ssRNA1 | 7a |
| | guide_88 | | ACGAAGGGGACTAAAACggc | ttgtgtggaa | CCCAAAAACGAA | | |
| | | | tcgtatgttgtgtggaattgtgagc | ttgtgagc | GGGGACTAAAAC | | |
| | | | (SEQ ID. NO: 642) | (SEQ ID. | (SEQ ID. | | |
| | | | | NO: 643) | NO: 644) | | |
| 7a | ssRNA1_ | LwaCas13a | GATTTAGACTACCCCAAAA | cggctcgtat | GATTTAGACTAC | ssRNA1 | 7a |
| | guide_89 | | ACGAAGGGGACTAAAACcgg | gttgtgtgga | CCCAAAAACGAA | | |
| | | | ctcgtatgttgtgtggaattgtgag | attgtgag | GGGGACTAAAAC | | |
| | | | (SEQ ID. NO: 645) | (SEQ ID. | (SEQ ID. | | |
| | | | | NO: 646) | NO: 647) | | |
| 7a | ssRNA1_ | LwaCas13a | GATTTAGACTACCCCAAAA | ccggctcgt | GATTTAGACTAC | ssRNA1 | 7a |
| | guide_90 | | ACGAAGGGGACTAAAACccg | atgttgtgtg | CCCAAAAACGAA | | |
| | | | gctcgtatgttgtgtggaattgtga | gaattgtga | GGGGACTAAAAC | | |
| | | | (SEQ ID. NO: 648) | (SEQ ID. | (SEQ ID. | | |
| | | | | NO: 649) | NO: 650) | | |
| 7a | ssRNA1_ | LwaCas13a | GATTTAGACTACCCCAAAA | ttccggctcg | GATTTAGACTAC | ssRNA1 | 7a |
| | guide_91 | | ACGAAGGGGACTAAAACttc | tatgttgtgtg | CCCAAAAACGAA | | |
| | | | cggctcgtatgttgtgtggaattgt | gaattgt | GGGGACTAAAAC | | |
| | | | (SEQ ID. NO: 651) | (SEQ ID. | (SEQ ID. | | |
| | | | | NO: 652) | NO: 653) | | |
| 7a | ssRNA1_ | LwaCas13a | GATTTAGACTACCCCAAAA | cttccggctc | GATTTAGACTAC | ssRNA1 | 7a |
| | guide_92 | | ACGAAGGGGACTAAAACctt | gtatgttgtgt | CCCAAAAACGAA | | |
| | | | ccggctcgtatgttgtgtggaattg | ggaattg | GGGGACTAAAAC | | |
| | | | (SEQ ID. NO: 654) | (SEQ ID. | (SEQ ID. | | |
| | | | | NO: 655) | NO: 656) | | |
| 7a | ssRNA1_ | LwaCas13a | GATTTAGACTACCCCAAAA | gcttccggct | GATTTAGACTAC | ssRNA1 | 7a |
| | guide_93 | | ACGAAGGGGACTAAAACgct | cgtatgttgt | CCCAAAAACGAA | | |
| | | | tccggctcgtatgttgtgtggaatt | gtggaatt | GGGGACTAAAAC | | |
| | | | (SEQ ID. NO: 657) | (SEQ ID. | (SEQ ID. | | |
| | | | | NO: 658) | NO: 659) | | |
| 7a | ssRNA1_ | LwaCas13a | GATTTAGACTACCCCAAAA | atgcttccgg | GATTTAGACTAC | ssRNA1 | 7a |
| | guide_94 | | ACGAAGGGGACTAAAACatg | ctcgtatgttg | CCCAAAAACGAA | | |
| | | | cttceggctcgtatgttgtgtggaa | tgtggaa | GGGGACTAAAAC | | |
| | | | (SEQ ID. NO: 660) | (SEQ ID. | (SEQ ID. | | |
| | | | | NO: 661) | NO: 662) | | |
| 7a | ssRNA1_ | LwaCas13a | GATTTAGACTACCCCAAAA | tatgcttccg | GATTTAGACTAC | ssRNA1 | 7a |
| | guide_95 | | ACGAAGGGGACTAAAACtat | gctcgtatgtt | CCCAAAAACGAA | | |
| | | | gcttccggctcgtatgttgtgtgga | gtgtgga | GGGGACTAAAAC | | |
| | | | (SEQ ID. NO: 663) | (SEQ ID. | (SEQ ID. | | |
| | | | | NO: 664) | NO: 665) | | |
| 7a | ssRNA1_ | LwaCas13a | GATTTAGACTACCCCAAAA | ttatgcttccg | GATTTAGACTAC | ssRNA1 | 7a |
| | guide_96 | | ACGAAGGGGACTAAAACtta | gctcgtatgtt | CCCAAAAACGAA | | |
| | | | tgcttccggctcgtatgttgtgtgg | gtgtgg | GGGGACTAAAAC | | |
| | | | (SEQ ID. NO: 666) | (SEQ ID. | (SEQ ID. | | |
| | | | | NO: 667) | NO: 668) | | |
| 7a | therm_00 | LwaCas13a | GATTTAGACTACCCCAAAA | taatttaaca | GATTTAGACTAC | ther- | 7a |
| | | | ACGAAGGGGACTAAAACtaa | gtatcaccat | CCCAAAAACGAA | mo- | |
| | | | tttaacagtatcaccatcaatcgct | caatcgct | GGGGACTAAAAC | nu- | |
| | | | (SEQ ID. NO: 669) | (SEQ ID. | (SEQ ID. | clease | |
| | | | | NO: 670) | NO: 671) | | |
| 7a | therm_01 | LwaCas13a | GATTTAGACTACCCCAAAA | ttaatttaaca | GATTTAGACTAC | ther- | 7a |
| | | | ACGAAGGGGACTAAAACtta | gtatcaccat | CCCAAAAACGAA | mo- | |
| | | | atttaacagtatcaccatcaatcgc | caatcgc | GGGGACTAAAAC | nu- | |
| | | | (SEQ ID. NO: 672) | (SEQ ID. | (SEQ ID. | clease | |
| | | | | NO: 673) | NO: 674) | | |
| 7a | therm_02 | LwaCas13a | GATTTAGACTACCCCAAAA | attaatttaac | GATTTAGACTAC | ther- | 7a |
| | | | ACGAAGGGGACTAAAACatt | agtatcacca | CCCAAAAACGAA | mo- | |
| | | | aatttaacagtatcaccatcaatcg | tcaatcg | GGGGACTAAAAC | nu- | |
| | | | (SEQ ID. NO: 675) | (SEQ ID. | (SEQ ID. | clease | |
| | | | | NO: 676) | NO: 677) | | |
| 7a | therm_03 | LwaCas13a | GATTTAGACTACCCCAAAA | cattaatttaa | GATTTAGACTAC | ther- | 7a |
| | | | ACGAAGGGGACTAAAACcat | cagtatcacc | CCCAAAAACGAA | mo- | |
| | | | taatttaacagtatcaccatcaatc | atcaatc | GGGGACTAAAAC | nu- | |
| | | | (SEQ ID. NO: 678) | (SEQ ID. | (SEQ ID. | clease | |
| | | | | NO: 679) | NO: 680) | | |
| 7a | therm_04 | LwaCas13a | GATTTAGACTACCCCAAAA | acattaattta | GATTTAGACTAC | ther- | 7a |
| | | | ACGAAGGGGACTAAAACaca | acagtatcac | CCCAAAAACGAA | mo- | |
| | | | ttaatttaacagtatcaccatcaat | catcaat | GGGGACTAAAAC | nu- | |
| | | | (SEQ ID. NO: 681) | (SEQ ID. | (SEQ ID. | clease | |
| | | | | NO: 682) | NO: 683) | | |
| 7a | therm_05 | LwaCas13a | GATTTAGACTACCCCAAAA | tacattaattt | GATTTAGACTAC | ther- | 7a |
| | | | ACGAAGGGGACTAAAACtac | aacagtatca | CCCAAAAACGAA | mo- | |
| | | | attaatttaacagtatcaccatcaa | ccatcaa | GGGGACTAAAAC | nu- | |
| | | | (SEQ ID. NO: 684) | (SEQ ID. | (SEQ ID. | clease | |
| | | | | NO: 685) | NO: 686) | | |
| 7a | therm_06 | LwaCas13a | GATTTAGACTACCCCAAAA | gtacattaatt | GATTTAGACTAC | ther- | 7a |
| | | | ACGAAGGGGACTAAAACgta | taacagtatc | CCCAAAAACGAA | mo- | |
| | | | cattaatttaacagtatcaccatca | accatca | GGGGACTAAAAC | nu- | |
| | | | (SEQ ID. NO: 687) | (SEQ ID. | (SEQ ID. | clease | |
| | | | | NO: 688) | NO: 689) | | |
| 7a | therm_07 | LwaCas13a | GATTTAGACTACCCCAAAA | tgtacattaat | GATTTAGACTAC | ther- | 7a |
| | | | ACGAAGGGGACTAAAACtgt | ttaacagtat | CCCAAAAACGAA | mo- | |
| | | | acattaatttaacagtatcaccatc | caccatc | GGGGACTAAAAC | nu- | |
| | | | (SEQ ID. NO: 690) | (SEQ ID. | (SEQ ID. | clease | |
| | | | | NO: 691) | NO: 692) | | |
| 7a | therm_08 | LwaCas13a | GATTTAGACTACCCCAAAA | ttgtacattaa | GATTTAGACTAC | ther- | 7a |
| | | | ACGAAGGGGACTAAAACttg | tttaacagtat | CCCAAAAACGAA | mo- | |
| | | | tacattaatttaacagtatcaccat | caccat | GGGGACTAAAAC | nu- | |
| | | | (SEQ ID. NO: 693) | (SEQ ID. | (SEQ ID. | clease | |
| | | | | NO: 694) | NO: 695) | | |
| 7a | therm_09 | LwaCas13a | GATTTAGACTACCCCAAAA | tttgtacatta | GATTTAGACTAC | ther- | 7a |
| | | | ACGAAGGGGACTAAAACttt | atttaacagt | CCCAAAAACGAA | mo- | |
| | | | gtacattaatttaacagtatcacca | atcacca | GGGGACTAAAAC | nu- | |
| | | | (SEQ ID. NO: 696) | (SEQ ID. | (SEQ ID. | clease | |
| | | | | NO: 697) | NO: 698) | | |
| 7a | therm_10 | LwaCas13a | GATTTAGACTACCCCAAAA | ctttgtacatt | GATTTAGACTAC | ther- | 7a |
| | | | ACGAAGGGGACTAAAACctt | aatttaacag | CCCAAAAACGAA | mo- | |
| | | | tgtacattaatttaacagtatcacc | tatcacc | GGGGACTAAAAC | nu- | |
| | | | (SEQ ID. NO: 699) | (SEQ ID. | (SEQ ID. | clease | |
| | | | | NO: 700) | NO: 701) | | |
| 7a | therm_11 | LwaCas13a | GATTTAGACTACCCCAAAA | cctttgtacat | GATTTAGACTAC | ther- | 7a |
| | | | ACGAAGGGGACTAAAACcct | taatttaaca | CCCAAAAACGAA | mo- | |
| | | | ttgtacattaatttaacagtatcac | gtatcac | GGGGACTAAAAC | nu- | |
| | | | (SEQ ID. NO: 702) | (SEQ ID. | (SEQ ID. | clease | |
| | | | | NO: 703) | NO: 704) | | |
| 7a | therm_12 | LwaCas13a | GATTTAGACTACCCCAAAA | acctttgtac | GATTTAGACTAC | ther- | 7a |
| | | | ACGAAGGGGACTAAAACacc | attaatttaac | CCCAAAAACGAA | mo- | |
| | | | tttgtacattaatttaacagtatca | agtatca | GGGGACTAAAAC | nu- | |
| | | | (SEQ ID. NO: 705) | (SEQ ID. | (SEQ ID. | clease | |
| | | | | NO: 706) | NO: 707) | | |
| 7a | therm_13 | LwaCas13a | GATTTAGACTACCCCAAAA | gacctttgta | GATTTAGACTAC | ther- | 7a |
| | | | ACGAAGGGGACTAAAACgac | cattaatttaa | CCCAAAAACGAA | mo- | |
| | | | ctttgtacattaatttaacagtatc | cagtatc | GGGGACTAAAAC | nu- | |
| | | | (SEQ ID. NO: 708) | (SEQ ID. | (SEQ ID. | clease | |
| | | | | NO: 709) | NO: 710) | | |
| 7a | therm_14 | LwaCas13a | GATTTAGACTACCCCAAAA | tgacctttgta | GATTTAGACTAC | ther- | 7a |
| | | | ACGAAGGGGACTAAAACtga | cattaatttaa | CCCAAAAACGAA | mo- | |
| | | | cctttgtacattaatttaacagtat | cagtat | GGGGACTAAAAC | nu- | |
| | | | (SEQ ID. NO: 711) | (SEQ ID. | (SEQ ID. | clease | |
| | | | | NO: 712) | NO: 713) | | |
| 7a | therm_15 | LwaCas13a | GATTTAGACTACCCCAAAA | ttgacctttgt | GATTTAGACTAC | ther- | 7a |
| | | | ACGAAGGGGACTAAAACttg | acattaattta | CCCAAAAACGAA | mo- | |
| | | | acctttgtacattaatttaacagta | acagta | GGGGACTAAAAC | nu- | |
| | | | (SEQ ID. NO: 714) | (SEQ ID. | (SEQ ID. | clease | |
| | | | | NO: 715) | NO: 716) | | |
| 7a | therm_16 | LwaCas13a | GATTTAGACTACCCCAAAA | gttgacctttg | GATTTAGACTAC | ther- | 7a |
| | | | ACGAAGGGGACTAAAACgtt | tacattaattt | CCCAAAAACGAA | mo- | |
| | | | gacctttgtacattaatttaacagt | aacagt | GGGGACTAAAAC | nu- | |
| | | | (SEQ ID. NO: 717) | (SEQ ID. | (SEQ ID. | clease | |
| | | | | NO: 718) | NO: 719) | | |
| 7a | therm_17 | LwaCas13a | GATTTAGACTACCCCAAAA | ggttgaccttt | GATTTAGACTAC | ther- | 7a |
| | | | ACGAAGGGGACTAAAACggt | gtacattaatt | CCCAAAAACGAA | mo- | |
| | | | tgacctttgtacattaatttaacag | taacag | GGGGACTAAAAC | nu- | |
| | | | (SEQ ID. NO: 720) | (SEQ ID. | (SEQ ID. | clease | |
| | | | | NO: 721) | NO: 722) | | |
| 7a | therm_18 | LwaCas13a | GATTTAGACTACCCCAAAA | tggttgacctt | GATTTAGACTAC | ther- | 7a |
| | | | ACGAAGGGGACTAAAACtgg | tgtacattaat | CCCAAAAACGAA | mo- | |
| | | | ttgacctttgtacattaatttaaca | ttaaca | GGGGACTAAAAC | nu- | |
| | | | (SEQ ID. NO: 723) | (SEQ ID. | (SEQ ID. | clease | |
| | | | | NO: 724) | NO: 725) | | |
| 7a | therm_19 | LwaCas13a | GATTTAGACTACCCCAAAA | ttggttgacct | GATTTAGACTAC | ther- | 7a |
| | | | ACGAAGGGGACTAAAACttg | ttgtacattaa | CCCAAAAACGAA | mo- | |
| | | | gttgacctttgtacattaatttaac | tttaac | GGGGACTAAAAC | nu- | |
| | | | (SEQ ID. NO: 726) | (SEQ ID. | (SEQ ID. | clease | |
| | | | | NO: 727) | NO: 728) | | |
| 7a | therm_20 | LwaCas13a | GATTTAGACTACCCCAAAA | cattggttga | GATTTAGACTAC | ther- | 7a |
| | | | ACGAAGGGGACTAAAACcat | cctttgtacat | CCCAAAAACGAA | mo- | |
| | | | tggttgacctttgtacattaattta | taattta | GGGGACTAAAAC | nu- | |
| | | | (SEQ ID. NO: 729) | (SEQ ID. | (SEQ ID. | clease | |
| | | | | NO: 730) | NO: 731) | | |
| 7a | therm_21 | LwaCas13a | GATTTAGACTACCCCAAAA | gtcattggtt | GATTTAGACTAC | ther- | 7a |
| | | | ACGAAGGGGACTAAAACgtc | gacctttgta | CCCAAAAACGAA | mo- | |
| | | | attggttgacctttgtacattaatt | cattaatt | GGGGACTAAAAC | nu- | |
| | | | (SEQ ID. NO: 732) | (SEQ ID. | (SEQ ID. | clease | |
| | | | | NO: 733) | NO: 734) | | |
| 7a | therm_22 | LwaCas13a | GATTTAGACTACCCCAAAA | atgtcattggt | GATTTAGACTAC | ther- | 7a |
| | | | ACGAAGGGGACTAAAACatg | tgacctttgta | CCCAAAAACGAA | mo- | |
| | | | tcattggttgacctttgtacattaa | cattaa | GGGGACTAAAAC | nu- | |
| | | | (SEQ ID. NO: 735) | (SEQ ID. | (SEQ ID. | clease | |
| | | | | NO: 736) | NO: 737) | | |
| 7a | therm_23 | LwaCas13a | GATTTAGACTACCCCAAAA | gaatgtcatt | GATTTAGACTAC | ther- | 7a |
| | | | ACGAAGGGGACTAAAACgaa | ggttgaccttt | CCCAAAAACGAA | mo- | |
| | | | tgtcattggttgacctttgtacatt | gtacatt | GGGGACTAAAAC | nu- | |
| | | | (SEQ ID. NO: 738) | (SEQ ID. | (SEQ ID. | clease | |
| | | | | NO: 739) | NO: 740) | | |
| 7a | therm_24 | LwaCas13a | GATTTAGACTACCCCAAAA | ctgaatgtca | GATTTAGACTAC | ther- | 7a |
| | | | ACGAAGGGGACTAAAACctg | ttggttgacct | CCCAAAAACGAA | mo- | |
| | | | aatgtcattggttgacctttgtaca | ttgtaca | GGGGACTAAAAC | nu- | |
| | | | (SEQ ID. NO: 741) | (SEQ ID. | (SEQ ID. | clease | |
| | | | | NO: 742) | NO: 743) | | |
| 7a | therm_25 | LwaCas13a | GATTTAGACTACCCCAAAA | gtctgaatgt | GATTTAGACTAC | ther- | 7a |
| | | | ACGAAGGGGACTAAAACgtc | cattggttga | CCCAAAAACGAA | mo- | |
| | | | tgaatgtcattggttgacctttgta | cctttgta | GGGGACTAAAAC | nu- | |
| | | | (SEQ ID. NO: 744) | (SEQ ID. | (SEQ ID. | clease | |
| | | | | NO: 745) | NO: 746) | | |
| 7a | therm_26 | LwaCas13a | GATTTAGACTACCCCAAAA | tagtctgaat | GATTTAGACTAC | ther- | 7a |
| | | | ACGAAGGGGACTAAAACtag | gtcattggtt | CCCAAAAACGAA | mo- | |
| | | | tctgaatgtcattggttgacctttg | gacctttg | GGGGACTAAAAC | nu- | |
| | | | (SEQ ID. NO: 747) | (SEQ ID. | (SEQ ID. | clease | |
| | | | | NO: 748) | NO: 749) | | |
| 7a | therm_27 | LwaCas13a | GATTTAGACTACCCCAAAA | aatagtctga | GATTTAGACTAC | ther- | 7a |
| | | | ACGAAGGGGACTAAAACaat | atgtcattggt | CCCAAAAACGAA | mo- | |
| | | | agtctgaatgtcattggttgacctt | tgacctt | GGGGACTAAAAC | nu- | |
| | | | (SEQ ID. NO: 750) | (SEQ ID. | (SEQ ID. | clease | |
| | | | | NO: 751) | NO: 752) | | |
| 7a | therm_28 | LwaCas13a | GATTTAGACTACCCCAAAA | ataatagtct | GATTTAGACTAC | ther- | 7a |
| | | | ACGAAGGGGACTAAAACata | gaatgtcatt | CCCAAAAACGAA | mo- | |
| | | | atagtctgaatgtcattggttgacc | ggttgacc | GGGGACTAAAAC | nu- | |
| | | | (SEQ ID. NO: 753) | (SEQ ID. | (SEQ ID. | clease | |
| | | | | NO: 754) | NO: 755) | | |
| 7a | therm_29 | LwaCas13a | GATTTAGACTACCCCAAAA | caataatagt | GATTTAGACTAC | ther- | 7a |
| | | | ACGAAGGGGACTAAAACcaa | ctgaatgtca | CCCAAAAACGAA | mo- | |
| | | | taatagtctgaatgtcattggttga | ttggttga | GGGGACTAAAAC | nu- | |
| | | | (SEQ ID. NO: 756) | (SEQ ID. | (SEQ ID. | clease | |
| | | | | NO: 757) | NO: 758) | | |
| 7a | therm_30 | LwaCas13a | GATTTAGACTACCCCAAAA | accaataata | GATTTAGACTAC | ther- | 7a |
| | | | ACGAAGGGGACTAAAACacc | gtctgaatgt | CCCAAAAACGAA | mo- | |
| | | | aataatagtctgaatgtcattggtt | cattggtt | GGGGACTAAAAC | nu- | |
| | | | (SEQ ID. NO: 759) | (SEQ ID. | (SEQ ID. | clease | |
| | | | | NO: 760) | NO: 761) | | |
| 7a | therm_31 | LwaCas13a | GATTTAGACTACCCCAAAA | caaccaata | GATTTAGACTAC | ther- | 7a |
| | | | ACGAAGGGGACTAAAACcaa | atagtctgaa | CCCAAAAACGAA | mo- | |
| | | | ccaataatagtctgaatgtcattgg | tgtcattgg | GGGGACTAAAAC | nu- | |
| | | | (SEQ ID. NO: 762) | (SEQ ID. | (SEQ ID. | clease | |
| | | | | NO: 763) | NO: 764) | | |
| 7a | therm_32 | LwaCas13a | GATTTAGACTACCCCAAAA | atcaaccaat | GATTTAGACTAC | ther- | 7a |
| | | | ACGAAGGGGACTAAAACatc | aatagtctga | CCCAAAAACGAA | mo- | |
| | | | aaccaataatagtctgaatgtcatt | atgtcatt | GGGGACTAAAAC | nu- | |
| | | | (SEQ ID. NO: 765) | (SEQ ID. | (SEQ ID. | clease | |
| | | | | NO: 766) | NO: 767) | | |
| 7a | therm_33 | LwaCas13a | GATTTAGACTACCCCAAAA | gtatcaacca | GATTTAGACTAC | ther- | 7a |
| | | | ACGAAGGGGACTAAAACgta | ataatagtct | CCCAAAAACGAA | mo- | |
| | | | tcaaccaataatagtctgaatgtca | gaatgtca | GGGGACTAAAAC | nu- | |
| | | | (SEQ ID. NO: 768) | (SEQ ID. | (SEQ ID. | clease | |
| | | | | NO: 769) | NO: 770) | | |
| 7a | therm_34 | LwaCas13a | GATTTAGACTACCCCAAAA | gtgtatcaac | GATTTAGACTAC | ther- | 7a |
| | | | ACGAAGGGGACTAAAACgtg | caataatagt | CCCAAAAACGAA | mo- | |
| | | | tatcaaccaataatagtctgaatgt | ctgaatgt | GGGGACTAAAAC | nu- | |
| | | | (SEQ ID. NO: 771) | (SEQ ID. | (SEQ ID. | clease | |
| | | | | NO: 772) | NO: 773) | | |
| 7a | therm_35 | LwaCas13a | GATTTAGACTACCCCAAAA | aggtgtatca | GATTTAGACTAC | ther- | 7a |
| | | | ACGAAGGGGACTAAAACagg | accaataata | CCCAAAAACGAA | mo- | |
| | | | tgtatcaaccaataatagtctgaat | gtctgaat | GGGGACTAAAAC | nu- | |
| | | | (SEQ ID. NO: 774) | (SEQ ID. | (SEQ ID. | clease | |
| | | | | NO: 775) | NO: 776) | | |
| 7a | therm_36 | LwaCas13a | GATTTAGACTACCCCAAAA | tcaggtgtat | GATTTAGACTAC | ther- | 7a |
| | | | ACGAAGGGGACTAAAACtca | caaccaata | CCCAAAAACGAA | mo- | |
| | | | ggtgtatcaaccaataatagtctga | atagtctga | GGGGACTAAAAC | nu- | |
| | | | (SEQ ID. NO: 777) | (SEQ ID. | (SEQ ID. | clease | |
| | | | | NO: 778) | NO: 779) | | |
| 7a | therm_37 | LwaCas13a | GATTTAGACTACCCCAAAA | tttcaggtgta | GATTTAGACTAC | ther- | 7a |
| | | | ACGAAGGGGACTAAAACttt | tcaaccaata | CCCAAAAACGAA | mo- | |
| | | | caggtgtatcaaccaataatagtct | atagtct | GGGGACTAAAAC | nu- | |
| | | | (SEQ ID. NO: 780) | (SEQ ID. | (SEQ ID. | clease | |
| | | | | NO: 781) | NO: 782) | | |
| 7a | therm_38 | LwaCas13a | GATTTAGACTACCCCAAAA | tgtttcaggt | GATTTAGACTAC | ther- | 7a |
| | | | ACGAAGGGGACTAAAACtgt | gtatcaacca | CCCAAAAACGAA | mo- | |
| | | | ttcaggtgtatcaaccaataatagt | ataatagt | GGGGACTAAAAC | nu- | |
| | | | (SEQ ID. NO: 783) | (SEQ ID. | (SEQ ID. | clease | |
| | | | | NO: 784) | NO: 785) | | |
| 7a | therm_39 | LwaCas13a | GATTTAGACTACCCCAAAA | tttgtttcagg | GATTTAGACTAC | ther- | 7a |
| | | | ACGAAGGGGACTAAAACttt | tgtatcaacc | CCCAAAAACGAA | mo- | |
| | | | gtttcaggtgtatcaaccaataata | aataata | GGGGACTAAAAC | nu- | |
| | | | (SEQ ID. NO: 786) | (SEQ ID. | (SEQ ID. | clease | |
| | | | | NO: 787) | NO: 788) | | |
| 7a | therm_40 | LwaCas13a | GATTTAGACTACCCCAAAA | gctttgtttca | GATTTAGACTAC | ther- | 7a |
| | | | ACGAAGGGGACTAAAACgct | ggtgtatcaa | CCCAAAAACGAA | mo- | |
| | | | ttgtttcaggtgtatcaaccaataa | ccaataa | GGGGACTAAAAC | nu- | |
| | | | (SEQ ID. NO: 789) | (SEQ ID. | (SEQ ID. | clease | |
| | | | | NO: 790) | NO: 791) | | |
| 7a | therm_41 | LwaCas13a | GATTTAGACTACCCCAAAA | atgctttgttt | GATTTAGACTAC | ther- | 7a |
| | | | ACGAAGGGGACTAAAACatg | caggtgtatc | CCCAAAAACGAA | mo- | |
| | | | ctttgtttcaggtgtatcaaccaat | aaccaat | GGGGACTAAAAC | nu- | |
| | | | (SEQ ID. NO: 792) | (SEQ ID. | (SEQ ID. | clease | |
| | | | | NO: 793) | NO: 794) | | |
| 7a | therm_42 | LwaCas13a | GATTTAGACTACCCCAAAA | ggatgctttg | GATTTAGACTAC | ther- | 7a |
| | | | ACGAAGGGGACTAAAACgga | tttcaggtgta | CCCAAAAACGAA | mo- | |
| | | | tgctttgtttcaggtgtatcaacca | tcaacca | GGGGACTAAAAC | nu- | |
| | | | (SEQ ID. NO: 795) | (SEQ ID. | (SEQ ID. | clease | |
| | | | | NO: 796) | NO: 797) | | |
| 7a | therm_43 | LwaCas13a | GATTTAGACTACCCCAAAA | taggatgcttt | GATTTAGACTAC | ther- | 7a |
| | | | ACGAAGGGGACTAAAACtag | gtttcaggtg | CCCAAAAACGAA | mo- | |
| | | | gatgctttgtttcaggtgtatcaac | tatcaac | GGGGACTAAAAC | nu- | |
| | | | (SEQ ID. NO: 798) | (SEQ ID. | (SEQ ID. | clease | |
| | | | | NO: 799) | NO: 800) | | |
| 7a | therm_44 | LwaCas13a | GATTTAGACTACCCCAAAA | tttaggatgct | GATTTAGACTAC | ther- | 7a |
| | | | ACGAAGGGGACTAAAACttt | ttgtttcaggt | CCCAAAAACGAA | mo- | |
| | | | aggatgctttgtttcaggtgtatca | gtatca | GGGGACTAAAAC | nu- | |
| | | | (SEQ ID. NO: 801) | (SEQ ID. | (SEQ ID. | clease | |
| | | | | NO: 802) | NO: 803) | | |
| 7a | therm_45 | LwaCas13a | GATTTAGACTACCCCAAAA | tttttaggatg | GATTTAGACTAC | ther- | 7a |
| | | | ACGAAGGGGACTAAAACtttt | ctttgtttcag | CCCAAAAACGAA | mo- | |
| | | | taggatgctttgtttcaggtgtat | gtgtat | GGGGACTAAAAC | nu- | |
| | | | (SEQ ID. NO: 804) | (SEQ ID. | (SEQ ID. | clease | |
| | | | | NO: 805) | NO: 806) | | |
| 7a | therm_46 | LwaCas13a | GATTTAGACTACCCCAAAA | cttttttagga | GATTTAGACTAC | ther- | 7a |
| | | | ACGAAGGGGACTAAAACctt | tgctttgtttc | CCCAAAAACGAA | mo- | |
| | | | ttttaggatgctttgtttcaggtgt | aggtgt | GGGGACTAAAAC | nu- | |
| | | | (SEQ ID. NO: 807) | (SEQ ID. | (SEQ ID. | clease | |
| | | | | NO: 808) | NO: 809) | | |
| 7a | therm_47 | LwaCas13a | GATTTAGACTACCCCAAAA | accttttttag | GATTTAGACTAC | ther- | 7a |
| | | | ACGAAGGGGACTAAAACacc | gatgctttgtt | CCCAAAAACGAA | mo- | |
| | | | ttttttaggatgctttgtttcaggt | tcaggt | GGGGACTAAAAC | nu- | |
| | | | (SEQ ID. NO: 810) | (SEQ ID. | (SEQ ID. | clease | |
| | | | | NO: 811) | NO: 812) | | |
| 7a | therm_48 | LwaCas13a | GATTTAGACTACCCCAAAA | acacctttttt | GATTTAGACTAC | ther- | 7a |
| | | | ACGAAGGGGACTAAAACaca | aggatgcttt | CCCAAAAACGAA | mo- | |
| | | | ccttttttaggatgctttgtttcag | gtttcag | GGGGACTAAAAC | nu- | |
| | | | (SEQ ID. NO: 813) | (SEQ ID. | (SEQ ID. | clease | |
| | | | | NO: 814) | NO: 815) | | |
| 7a | therm_49 | LwaCas13a | GATTTAGACTACCCCAAAA | ctacacctttt | GATTTAGACTAC | ther- | 7a |
| | | | ACGAAGGGGACTAAAACcta | ttaggatgctt | CCCAAAAACGAA | mo- | |
| | | | caccttttttaggatgctttgtttc | tgtttc | GGGGACTAAAAC | nu- | |
| | | | (SEQ ID. NO: 816) | (SEQ ID. | (SEQ ID. | clease | |
| | | | | NO: 817) | NO: 818) | | |
| 7a | therm_50 | LwaCas13a | GATTTAGACTACCCCAAAA | ctctacacctt | GATTTAGACTAC | ther- | 7a |
| | | | ACGAAGGGGACTAAAACctc | ttttaggatgc | CCCAAAAACGAA | mo- | |
| | | | tacaccttttttaggatgctttgtt | tttgtt | GGGGACTAAAAC | nu- | |
| | | | (SEQ ID. NO: 819) | (SEQ ID. | (SEQ ID. | clease | |
| | | | | NO: 820) | NO: 821) | | |
| 7a | therm_51 | LwaCas13a | GATTTAGACTACCCCAAAA | ttctctacacc | GATTTAGACTAC | ther- | 7a |
| | | | ACGAAGGGGACTAAAACttc | ttttttaggat | CCCAAAAACGAA | mo- | |
| | | | tctacaccttttttaggatgctttg | gctttg | GGGGACTAAAAC | nu- | |
| | | | (SEQ ID. NO: 822) | (SEQ ID. | (SEQ ID. | clease | |
| | | | | NO: 823) | NO: 824) | | |
| 7a | therm_52 | LwaCas13a | GATTTAGACTACCCCAAAA | atttctctaca | GATTTAGACTAC | ther- | 7a |
| | | | ACGAAGGGGACTAAAACatt | ccttttttagg | CCCAAAAACGAA | mo- | |
| | | | tctctacaccttttttaggatgctt | atgctt | GGGGACTAAAAC | nu- | |
| | | | (SEQ ID. NO: 825) | (SEQ ID. | (SEQ ID. | clease | |
| | | | | NO: 826) | NO: 827) | | |
| 7a | therm_53 | LwaCas13a | GATTTAGACTACCCCAAAA | atatttctcta | GATTTAGACTAC | ther- | 7a |
| | | | ACGAAGGGGACTAAAACata | cacctttttta | CCCAAAAACGAA | mo- | |
| | | | tttctctacaccttttttaggatgc | ggatgc | GGGGACTAAAAC | nu- | |
| | | | (SEQ ID. NO: 828) | (SEQ ID. | (SEQ ID. | clease | |
| | | | | NO: 829) | NO: 830) | | |
| 7a | therm_54 | LwaCas13a | GATTTAGACTACCCCAAAA | ccatatttctc | GATTTAGACTAC | ther- | 7a |
| | | | ACGAAGGGGACTAAAACcca | tacaccttttt | CCCAAAAACGAA | mo- | |
| | | | tatttctctacaccttttttaggat | taggat | GGGGACTAAAAC | nu- | |
| | | | (SEQ ID. NO: 831) | (SEQ ID. | (SEQ ID. | clease | |
| | | | | NO: 832) | NO: 833) | | |
| 7a | therm_55 | LwaCas13a | GATTTAGACTACCCCAAAA | gaccatattt | GATTTAGACTAC | ther- | 7a |
| | | | ACGAAGGGGACTAAAACgac | ctctacacctt | CCCAAAAACGAA | mo- | |
| | | | catatttctctacaccttttttagg | ttttagg | GGGGACTAAAAC | nu- | |
| | | | (SEQ ID. NO: 834) | (SEQ ID. | (SEQ ID. | clease | |
| | | | | NO: 835) | NO: 836) | | |
| 7a | therm_56 | LwaCas13a | GATTTAGACTACCCCAAAA | aggaccatat | GATTTAGACTAC | ther- | 7a |
| | | | ACGAAGGGGACTAAAACagg | ttctctacacc | CCCAAAAACGAA | mo- | |
| | | | accatatttctctacacctttttta | tttttta | GGGGACTAAAAC | nu- | |
| | | | (SEQ ID. NO: 837) | (SEQ ID. | (SEQ ID. | clease | |
| | | | | NO: 838) | NO: 839) | | |
| 7a | therm_57 | LwaCas13a | GATTTAGACTACCCCAAAA | tcaggaccat | GATTTAGACTAC | ther- | 7a |
| | | | ACGAAGGGGACTAAAACtca | atttctctaca | CCCAAAAACGAA | mo- | |
| | | | ggaccatatttctctacaccttttt | ccttttt | GGGGACTAAAAC | nu- | |
| | | | (SEQ ID. NO: 840) | (SEQ ID. | (SEQ ID. | clease | |
| | | | | NO: 841) | NO: 842) | | |
| 7a | therm_58 | LwaCas13a | GATTTAGACTACCCCAAAA | cttcaggacc | GATTTAGACTAC | ther- | 7a |
| | | | ACGAAGGGGACTAAAACctt | atatttctcta | CCCAAAAACGAA | mo- | |
| | | | caggaccatatttctctacaccttt | caccttt | GGGGACTAAAAC | nu- | |
| | | | (SEQ ID. NO: 843) | (SEQ ID. | (SEQ ID. | clease | |
| | | | | NO: 844) | NO: 845) | | |
| 7a | therm_59 | LwaCas13a | GATTTAGACTACCCCAAAA | tgcttcagga | GATTTAGACTAC | ther- | 7a |
| | | | ACGAAGGGGACTAAAACtgc | ccatatttctc | CCCAAAAACGAA | mo- | |
| | | | ttcaggaccatatttctctacacct | tacacct | GGGGACTAAAAC | nu- | |
| | | | (SEQ ID. NO: 846) | (SEQ ID. | (SEQ ID. | clease | |
| | | | | NO: 847) | NO: 848) | | |
| 7a | therm_60 | LwaCas13a | GATTTAGACTACCCCAAAA | cttgcttcag | GATTTAGACTAC | ther- | 7a |
| | | | ACGAAGGGGACTAAAACctt | gaccatattt | CCCAAAAACGAA | mo- | |
| | | | gcttcaggaccatatttctctacac | ctctacac | GGGGACTAAAAC | nu- | |
| | | | (SEQ ID. NO: 849) | (SEQ ID. | (SEQ ID. | clease | |
| | | | | NO: 850) | NO: 851) | | |
| 7a | therm_61 | LwaCas13a | GATTTAGACTACCCCAAAA | cacttgcttc | GATTTAGACTAC | ther- | 7a |
| | | | ACGAAGGGGACTAAAACcac | aggaccatat | CCCAAAAACGAA | mo- | |
| | | | ttgcttcaggaccatatttctctac | ttctctac | GGGGACTAAAAC | nu- | |
| | | | (SEQ ID. NO: 852) | (SEQ ID. | (SEQ ID. | clease | |
| | | | | NO: 853) | NO: 854) | | |
| 7a | therm_62 | LwaCas13a | GATTTAGACTACCCCAAAA | tgcacttgctt | GATTTAGACTAC | ther- | 7a |
| | | | ACGAAGGGGACTAAAACtgc | caggaccat | CCCAAAAACGAA | mo- | |
| | | | acttgcttcaggaccatatttctct | atttctct | GGGGACTAAAAC | nu- | |
| | | | (SEQ ID. NO: 855) | (SEQ ID. | (SEQ ID. | clease | |
| | | | | NO: 856) | NO: 857) | | |
| 7a | therm_63 | LwaCas13a | GATTTAGACTACCCCAAAA | aatgcacttg | GATTTAGACTAC | ther- | 7a |
| | | | ACGAAGGGGACTAAAACaat | cttcaggacc | CCCAAAAACGAA | mo- | |
| | | | gcacttgcttcaggaccatatttct | atatttct | GGGGACTAAAAC | nu- | |
| | | | (SEQ ID. NO: 858) | (SEQ ID. | (SEQ ID. | clease | |
| | | | | NO: 859) | NO: 860) | | |
| 7a | therm_64 | LwaCas13a | GATTTAGACTACCCCAAAA | taaatgcact | GATTTAGACTAC | ther- | 7a |
| | | | ACGAAGGGGACTAAAACtaa | tgcttcagga | CCCAAAAACGAA | mo- | |
| | | | atgcacttgcttcaggaccatattt | ccatattt | GGGGACTAAAAC | nu- | |
| | | | (SEQ ID. NO: 861) | (SEQ ID. | (SEQ ID. | clease | |
| | | | | NO: 862) | NO: 863) | | |
| 7a | therm_65 | LwaCas13a | GATTTAGACTACCCCAAAA | gtaaatgcac | GATTTAGACTAC | ther- | 7a |
| | | | ACGAAGGGGACTAAAACgta | ttgcttcagg | CCCAAAAACGAA | mo- | |
| | | | aatgcacttgcttcaggaccatatt | accatatt | GGGGACTAAAAC | nu- | |
| | | | (SEQ ID. NO: 864) | (SEQ ID. | (SEQ ID. | clease | |
| | | | | NO: 865) | NO: 866) | | |
| 7a | therm_66 | LwaCas13a | GATTTAGACTACCCCAAAA | cgtaaatgca | GATTTAGACTAC | ther- | 7a |
| | | | ACGAAGGGGACTAAAACcgt | cttgcttcag | CCCAAAAACGAA | mo- | |
| | | | aaatgcacttgcttcaggaccatat | gaccatat | GGGGACTAAAAC | nu- | |
| | | | (SEQ ID. NO: 867) | (SEQ ID. | (SEQ ID. | clease | |
| | | | | NO: 868) | NO: 869) | | |
| 7a | therm_67 | LwaCas13a | GATTTAGACTACCCCAAAA | tcgtaaatgc | GATTTAGACTAC | ther- | 7a |
| | | | ACGAAGGGGACTAAAACtcg | acttgcttca | CCCAAAAACGAA | mo- | |
| | | | taaatgcacttgcttcaggaccata | ggaccata | GGGGACTAAAAC | nu- | |
| | | | (SEQ ID. NO: 870) | (SEQ ID. | (SEQ ID. | clease | |
| | | | | NO: 871) | NO: 872) | | |
| 7a | therm_68 | LwaCas13a | GATTTAGACTACCCCAAAA | ttcgtaaatg | GATTTAGACTAC | ther- | 7a |
| | | | ACGAAGGGGACTAAAACttc | cacttgcttc | CCCAAAAACGAA | mo- | |
| | | | gtaaatgcacttgcttcaggaccat | aggaccat | GGGGACTAAAAC | nu- | |
| | | | (SEQ ID. NO: 873) | (SEQ ID. | (SEQ ID. | clease | |
| | | | | NO: 874) | NO: 875) | | |
| 7a | therm_69 | LwaCas13a | GATTTAGACTACCCCAAAA | tttcgtaaatg | GATTTAGACTAC | ther- | 7a |
| | | | ACGAAGGGGACTAAAACttt | cacttgcttc | CCCAAAAACGAA | mo- | |
| | | | cgtaaatgcacttgcttcaggacca | aggacca | GGGGACTAAAAC | nu- | |
| | | | (SEQ ID. NO: 876) | (SEQ ID. | (SEQ ID. | clease | |
| | | | | NO: 877) | NO: 878) | | |
| 7a | therm_70 | LwaCas13a | GATTTAGACTACCCCAAAA | ttttcgtaaat | GATTTAGACTAC | ther- | 7a |
| | | | ACGAAGGGGACTAAAACtttt | gcacttgctt | CCCAAAAACGAA | mo- | |
| | | | cgtaaatgcacttgcttcaggacc | caggacc | GGGGACTAAAAC | nu- | |
| | | | (SEQ ID. NO: 879) | (SEQ ID. | (SEQ ID. | clease | |
| | | | | NO: 880) | NO: 881) | | |
| 7a | therm_71 | LwaCas13a | GATTTAGACTACCCCAAAA | tttttcgtaaa | GATTTAGACTAC | ther- | 7a |
| | | | ACGAAGGGGACTAAAACtttt | tgcacttgctt | CCCAAAAACGAA | mo- | |
| | | | tegtaaatgcacttgcttcaggac | caggac | GGGGACTAAAAC | nu- | |
| | | | (SEQ ID. NO: 882) | (SEQ ID. | (SEQ ID. | clease | |
| | | | | NO: 883) | NO: 884) | | |
| 7a | therm_72 | LwaCas13a | GATTTAGACTACCCCAAAA | ctttttcgtaa | GATTTAGACTAC | ther- | 7a |
| | | | ACGAAGGGGACTAAAACctt | atgcacttgc | CCCAAAAACGAA | mo- | |
| | | | tttcgtaaatgcacttgcttcagga | ttcagga | GGGGACTAAAAC | nu- | |
| | | | (SEQ ID. NO: 885) | (SEQ ID. | (SEQ ID. | clease | |
| | | | | NO: 886) | NO: 887) | | |
| 7a | therm_73 | LwaCas13a | GATTTAGACTACCCCAAAA | tctttttcgta | GATTTAGACTAC | ther- | 7a |
| | | | ACGAAGGGGACTAAAACtct | aatgcacttgc | CCCAAAAACGAA | mo- | |
| | | | ttttcgtaaatgcacttgcttcagg | ttcagg | GGGGACTAAAAC | nu- | |
| | | | (SEQ ID. NO: 888) | (SEQ ID. | (SEQ ID. | clease | |
| | | | | NO: 889) | NO: 890) | | |
| 7a | therm_74 | LwaCas13a | GATTTAGACTACCCCAAAA | atctttttcgt | GATTTAGACTAC | ther- | 7a |
| | | | ACGAAGGGGACTAAAACatc | aaatgcacttg | CCCAAAAACGAA | mo- | |
| | | | tttttcgtaaatgcacttgcttcag | cttcag | GGGGACTAAAAC | nu- | |
| | | | (SEQ ID. NO: 891) | (SEQ ID. | (SEQ ID. | clease | |
| | | | | NO: 892) | NO: 893) | | |
| 7a | therm_75 | LwaCas13a | GATTTAGACTACCCCAAAA | catctttttcg | GATTTAGACTAC | ther- | 7a |
| | | | ACGAAGGGGACTAAAACcat | taaatgcactt | CCCAAAAACGAA | mo- | |
| | | | ctttttcgtaaatgcacttgcttca | gcttca | GGGGACTAAAAC | nu- | |
| | | | (SEQ ID. NO: 894) | (SEQ ID. | (SEQ ID. | clease | |
| | | | | NO: 895) | NO: 896) | | |
| 7a | therm_76 | LwaCas13a | GATTTAGACTACCCCAAAA | ccatctttttc | GATTTAGACTAC | ther- | 7a |
| | | | ACGAAGGGGACTAAAACcca | gtaaatgcac | CCCAAAAACGAA | mo- | |
| | | | tctttttcgtaaatgcacttgcttc | ttgcttc | GGGGACTAAAAC | nu- | |
| | | | (SEQ ID. NO: 897) | (SEQ ID. | (SEQ ID. | clease | |
| | | | | NO: 898) | NO: 899) | | |
| 7a | therm_77 | LwaCas13a | GATTTAGACTACCCCAAAA | accatcttttt | GATTTAGACTAC | ther- | 7a |
| | | | ACGAAGGGGACTAAAACacc | cgtaaatgca | CCCAAAAACGAA | mo- | |
| | | | atctttttcgtaaatgcacttgctt | cttgctt | GGGGACTAAAAC | nu- | |
| | | | (SEQ ID. NO: 900) | (SEQ ID. | (SEQ ID. | clease | |
| | | | | NO: 901) | NO: 902) | | |
| 7a | therm_78 | LwaCas13a | GATTTAGACTACCCCAAAA | taccatctttt | GATTTAGACTAC | ther- | 7a |
| | | | ACGAAGGGGACTAAAACtac | tcgtaaatgca | CCCAAAAACGAA | mo- | |
| | | | catctttttcgtaaatgcacttgct | cttgct | GGGGACTAAAAC | nu- | |
| | | | (SEQ ID. NO: 903) | (SEQ ID. | (SEQ ID. | clease | |
| | | | | NO: 904) | NO: 905) | | |
| 7a | therm_79 | LwaCas13a | GATTTAGACTACCCCAAAA | ctaccatcttt | GATTTAGACTAC | ther- | 7a |
| | | | ACGAAGGGGACTAAAACcta | ttcgtaaatg | CCCAAAAACGAA | mo- | |
| | | | ccatctttttcgtaaatgcacttgc | cacttgc | GGGGACTAAAAC | nu- | |
| | | | (SEQ ID. NO: 906) | (SEQ ID. | (SEQ ID. | clease | |
| | | | | NO: 907) | NO: 908) | | |
| 7a | therm_80 | LwaCas13a | GATTTAGACTACCCCAAAA | tctaccatctt | GATTTAGACTAC | ther- | 7a |
| | | | ACGAAGGGGACTAAAACtct | tttcgtaaatg | CCCAAAAACGAA | mo- | |
| | | | accatctttttcgtaaatgcacttg | cacttg | GGGGACTAAAAC | nu- | |
| | | | (SEQ ID. NO: 909) | (SEQ ID. | (SEQ ID. | clease | |
| | | | | NO: 910) | NO: 911) | | |
| 7a | therm_81 | LwaCas13a | GATTTAGACTACCCCAAAA | ttctaccatct | GATTTAGACTAC | ther- | 7a |
| | | | ACGAAGGGGACTAAAACttc | ttttcgtaaat | CCCAAAAACGAA | mo- | |
| | | | taccatctttttcgtaaatgcactt | gcactt | GGGGACTAAAAC | nu- | |
| | | | (SEQ ID. NO: 912) | (SEQ ID. | (SEQ ID. | clease | |
| | | | | NO: 913) | NO: 914) | | |
| 7a | therm_82 | LwaCas13a | GATTTAGACTACCCCAAAA | tttctaccatc | GATTTAGACTAC | ther- | 7a |
| | | | ACGAAGGGGACTAAAACttt | tttttcgtaaa | CCCAAAAACGAA | mo- | |
| | | | ctaccatctttttcgtaaatgcact | tgcact | GGGGACTAAAAC | nu- | |
| | | | (SEQ ID. NO: 915) | (SEQ ID. | (SEQ ID. | clease | |
| | | | | NO: 916) | NO: 917) | | |
| 7a | therm_83 | LwaCas13a | GATTTAGACTACCCCAAAA | ttttctaccat | GATTTAGACTAC | ther- | 7a |
| | | | ACGAAGGGGACTAAAACtttt | ctttttcgtaa | CCCAAAAACGAA | mo- | |
| | | | ctaccatctttttcgtaaatgcac | atgcac | GGGGACTAAAAC | nu- | |
| | | | (SEQ ID. NO: 918) | (SEQ ID. | (SEQ ID. | clease | |
| | | | | NO: 919) | NO: 920) | | |
| 7a | therm_84 | LwaCas13a | GATTTAGACTACCCCAAAA | attttctacca | GATTTAGACTAC | ther- | 7a |
| | | | ACGAAGGGGACTAAAACatt | tctttttcgta | CCCAAAAACGAA | mo- | |
| | | | ttctaccatctttttcgtaaatgca | aatgca | GGGGACTAAAAC | nu- | |
| | | | (SEQ ID. NO: 921) | (SEQ ID. | (SEQ ID. | clease | |
| | | | | NO: 922) | NO: 923) | | |
| 7a | therm_85 | LwaCas13a | GATTTAGACTACCCCAAAA | cattttctacc | GATTTAGACTAC | ther- | 7a |
| | | | ACGAAGGGGACTAAAACcat | atctttttcgt | CCCAAAAACGAA | mo- | |
| | | | tttctaccatctttttcgtaaatgc | aaatgc | GGGGACTAAAAC | nu- | |
| | | | (SEQ ID. NO: 924) | (SEQ ID. | (SEQ ID. | clease | |
| | | | | NO: 925) | NO: 926) | | |
| 7a | therm_86 | LwaCas13a | GATTTAGACTACCCCAAAA | gcattttctac | GATTTAGACTAC | ther- | 7a |
| | | | ACGAAGGGGACTAAAACgca | catctttttcg | CCCAAAAACGAA | mo- | |
| | | | ttttctaccatctttttcgtaaatg | taaatg | GGGGACTAAAAC | nu- | |
| | | | (SEQ ID. NO: 927) | (SEQ ID. | (SEQ ID. | clease | |
| | | | | NO: 928) | NO: 929) | | |
| 7a | therm_87 | LwaCas13a | GATTTAGACTACCCCAAAA | tgcattttcta | GATTTAGACTAC | ther- | 7a |
| | | | ACGAAGGGGACTAAAACtgc | ccatctttttc | CCCAAAAACGAA | mo- | |
| | | | attttctaccatctttttcgtaaat | gtaaat | GGGGACTAAAAC | nu- | |
| | | | (SEQ ID. NO: 930) | (SEQ ID. | (SEQ ID. | clease | |
| | | | | NO: 931) | NO: 932) | | |
| 7a | therm_88 | LwaCas13a | GATTTAGACTACCCCAAAA | ttgcattttct | GATTTAGACTAC | ther- | 7a |
| | | | ACGAAGGGGACTAAAACttg | accatcttttt | CCCAAAAACGAA | mo- | |
| | | | cattttctaccatctttttcgtaaa | cgtaaa | GGGGACTAAAAC | nu- | |
| | | | (SEQ ID. NO: 933) | (SEQ ID. | (SEQ ID. | clease | |
| | | | | NO: 934) | NO: 935) | | |
| 7a | therm_89 | LwaCas13a | GATTTAGACTACCCCAAAA | tttgcattttc | GATTTAGACTAC | ther- | 7a |
| | | | ACGAAGGGGACTAAAACttt | taccatctttt | CCCAAAAACGAA | mo- | |
| | | | gcattttctaccatctttttcgtaa | tcgtaa | GGGGACTAAAAC | nu- | |
| | | | (SEQ ID. NO: 936) | (SEQ ID. | (SEQ ID. | clease | |
| | | | | NO: 937) | NO: 938) | | |
| 7a | therm_90 | LwaCas13a | GATTTAGACTACCCCAAAA | ctttgcatttt | GATTTAGACTAC | ther- | 7a |
| | | | ACGAAGGGGACTAAAACctt | ctaccatcttt | CCCAAAAACGAA | mo- | |
| | | | tgcattttctaccatctttttcgta | ttcgta | GGGGACTAAAAC | nu- | |
| | | | (SEQ ID. NO: 939) | (SEQ ID. | (SEQ ID. | clease | |
| | | | | NO: 940) | NO: 941) | | |
| 7a | therm_91 | LwaCas13a | GATTTAGACTACCCCAAAA | tctttgcattt | GATTTAGACTAC | ther- | 7a |
| | | | ACGAAGGGGACTAAAACtct | tctaccatctt | CCCAAAAACGAA | mo- | |
| | | | ttgcattttctaccatctttttcgt | tttcgt | GGGGACTAAAAC | nu- | |
| | | | (SEQ ID. NO: 942) | (SEQ ID. | (SEQ ID. | clease | |
| | | | | NO: 943) | NO: 944) | | |
| 7a | therm_92 | LwaCas13a | GATTTAGACTACCCCAAAA | ttctttgcatt | GATTTAGACTAC | ther- | 7a |
| | | | ACGAAGGGGACTAAAACttc | ttctaccatct | CCCAAAAACGAA | mo- | |
| | | | tttgcattttctaccatctttttcg | ttttcg | GGGGACTAAAAC | nu- | |
| | | | (SEQ ID. NO: 945) | (SEQ ID. | (SEQ ID. | clease | |
| | | | | NO: 946) | NO: 947) | | |
| 7a | therm_93 | LwaCas13a | GATTTAGACTACCCCAAAA | tttctttgcat | GATTTAGACTAC | ther- | 7a |
| | | | ACGAAGGGGACTAAAACttt | tttctaccatc | CCCAAAAACGAA | mo- | |
| | | | ctttgcattttctaccatctttttc | tttttc | GGGGACTAAAAC | nu- | |
| | | | (SEQ ID. NO: 948) | (SEQ ID. | (SEQ ID. | clease | |
| | | | | NO: 949) | NO: 950) | | |
| 7a | therm_94 | LwaCas13a | GATTTAGACTACCCCAAAA | ttttctttgca | GATTTAGACTAC | ther- | 7a |
| | | | ACGAAGGGGACTAAAACtttt | ttttctaccat | CCCAAAAACGAA | mo- | |
| | | | ctttgcattttctaccatcttttt | cttttt | GGGGACTAAAAC | nu- | |
| | | | (SEQ ID. NO: 951) | (SEQ ID. | (SEQ ID. | clease | |
| | | | | NO: 952) | NO: 953) | | |
| 7a | therm_95 | LwaCas13a | GATTTAGACTACCCCAAAA | attttctttgc | GATTTAGACTAC | ther- | 7a |
| | | | ACGAAGGGGACTAAAACatt | attttctacca | CCCAAAAACGAA | mo- | |
| | | | ttctttgcattttctaccatctttt | tctttt | GGGGACTAAAAC | nu- | |
| | | | (SEQ ID. NO: 954) | (SEQ ID. | (SEQ ID. | clease | |
| | | | | NO: 955) | NO: 956) | | |
| 1b | zika_00 | LwaCas13a | GATTTAGACTACCCCAAAA | tgttgttccag | GATTTAGACTAC | Zika | 1b |
| | | | ACGAAGGGGACTAAAACtgt | tgtggagttc | CCCAAAAACGAA | ssRNA | |
| | | | tgttccagtgtggagttccggtgtc | cggtgtc | GGGGACTAAAAC | | |
| | | | (SEQ ID. NO: 957) | (SEQ ID. | (SEQ ID. | | |
| | | | | NO: 958) | NO: 959) | | |
| 1b | zika_01 | LwaCas13a | GATTTAGACTACCCCAAAA | ttgttgttcca | GATTTAGACTAC | Zika | 1b |
| | | | ACGAAGGGGACTAAAACttg | gtgtggagtt | CCCAAAAACGAA | ssRNA | |
| | | | ttgttccagtgtggagttccggtgt | ccggtgt | GGGGACTAAAAC | | |
| | | | (SEQ ID. NO: 960) | (SEQ ID. | (SEQ ID. | | |
| | | | | NO: 961) | NO: 962) | | |
| 1b | zika_02 | LwaCas13a | GATTTAGACTACCCCAAAA | tttgttgttcc | GATTTAGACTAC | Zika | 1b |
| | | | ACGAAGGGGACTAAAACttt | agtgtggagt | CCCAAAAACGAA | ssRNA | |
| | | | gttgttccagtgtggagttccggtg | tccggtg | GGGGACTAAAAC | | |
| | | | (SEQ ID. NO: 963) | (SEQ ID. | (SEQ ID. | | |
| | | | | NO: 964) | NO: 965) | | |
| 1b | zika_03 | LwaCas13a | GATTTAGACTACCCCAAAA | ctttgttgttc | GATTTAGACTAC | Zika | 1b |
| | | | ACGAAGGGGACTAAAACctt | cagtgtgga | CCCAAAAACGAA | ssRNA | |
| | | | tgttgttccagtgtggagttccggt | gttccggt | GGGGACTAAAAC | | |
| | | | (SEQ ID. NO: 966) | (SEQ ID. | (SEQ ID. | | |
| | | | | NO: 967) | NO: 968) | | |
| 1b | zika_04 | LwaCas13a | GATTTAGACTACCCCAAAA | tctttgttgtt | GATTTAGACTAC | Zika | 1b |
| | | | ACGAAGGGGACTAAAACtct | ccagtgtgga | CCCAAAAACGAA | ssRNA | |
| | | | ttgttgttccagtgtggagttccgg | gttccgg | GGGGACTAAAAC | | |
| | | | (SEQ ID. NO: 969) | (SEQ ID. | (SEQ ID. | | |
| | | | | NO: 970) | NO: 971) | | |
| 1b | zika_05 | LwaCas13a | GATTTAGACTACCCCAAAA | ttctttgttgt | GATTTAGACTAC | Zika | 1b |
| | | | ACGAAGGGGACTAAAACttc | tccagtgtgg | CCCAAAAACGAA | ssRNA | |
| | | | tttgttgttccagtgtggagttccg | agttccg | GGGGACTAAAAC | | |
| | | | (SEQ ID. NO: 972) | (SEQ ID. | (SEQ ID. | | |
| | | | | NO: 973) | NO: 974) | | |
| 1b | zika_06 | LwaCas13a | GATTTAGACTACCCCAAAA | cttctttgttg | GATTTAGACTAC | Zika | 1b |
| | | | ACGAAGGGGACTAAAACctt | ttccagtgtgg | CCCAAAAACGAA | ssRNA | |
| | | | ctttgttgttccagtgtggagttcc | agttcc | GGGGACTAAAAC | | |
| | | | (SEQ ID. NO: 975) | (SEQ ID. | (SEQ ID. | | |
| | | | | NO: 976) | NO: 977) | | |
| 1b | zika_07 | LwaCas13a | GATTTAGACTACCCCAAAA | gcttctttgtt | GATTTAGACTAC | Zika | 1b |
| | | | ACGAAGGGGACTAAAACgct | gttccagtgt | CCCAAAAACGAA | ssRNA | |
| | | | tetttgttgttccagtgtggagttc | ggagttc | GGGGACTAAAAC | | |
| | | | (SEQ ID. NO: 978) | (SEQ ID. | (SEQ ID. | | |
| | | | | NO: 979) | NO: 980) | | |
| 1b | zika_08 | LwaCas13a | GATTTAGACTACCCCAAAA | tgcttctttgt | GATTTAGACTAC | Zika | 1b |
| | | | ACGAAGGGGACTAAAACtgc | tgttccagtgt | CCCAAAAACGAA | ssRNA | |
| | | | ttctttgttgttccagtgtggagtt | ggagtt | GGGGACTAAAAC | | |
| | | | (SEQ ID. NO: 981) | (SEQ ID. | (SEQ ID. | | |
| | | | | NO: 982) | NO: 983) | | |
| 1b | zika_09 | LwaCas13a | GATTTAGACTACCCCAAAA | gtgcttctttg | GATTTAGACTAC | Zika | 1b |
| | | | ACGAAGGGGACTAAAACgtg | ttgttccagtg | CCCAAAAACGAA | ssRNA | |
| | | | cttctttgttgttccagtgtggagt | tggagt | GGGGACTAAAAC | | |
| | | | (SEQ ID. NO: 984) | (SEQ ID. | (SEQ ID. | | |
| | | | | NO: 985) | NO: 986) | | |
| 1b | zika_10 | LwaCas13a | GATTTAGACTACCCCAAAA | agtgcttcttt | GATTTAGACTAC | Zika | 1b |
| | | | ACGAAGGGGACTAAAACagt | gttgttccagt | CCCAAAAACGAA | ssRNA | |
| | | | gcttctttgttgttccagtgtggag | gtggag | GGGGACTAAAAC | | |
| | | | (SEQ ID. NO: 987) | (SEQ ID. | (SEQ ID. | | |
| | | | | NO: 988) | NO: 989) | | |
| 1b | zika_11 | LwaCas13a | GATTTAGACTACCCCAAAA | cagtgcttctt | GATTTAGACTAC | Zika | 1b |
| | | | ACGAAGGGGACTAAAACcag | tgttgttccag | CCCAAAAACGAA | ssRNA | |
| | | | tgcttctttgttgttccagtgtgga | tgtgga | GGGGACTAAAAC | | |
| | | | (SEQ ID. NO: 990) | (SEQ ID. | (SEQ ID. | | |
| | | | | NO: 991) | NO: 992) | | |
| 1b | zika_12 | LwaCas13a | GATTTAGACTACCCCAAAA | ccagtgcttc | GATTTAGACTAC | Zika | 1b |
| | | | ACGAAGGGGACTAAAACcca | tttgttgttcc | CCCAAAAACGAA | ssRNA | |
| | | | gtgcttctttgttgttccagtgtgg | agtgtgg | GGGGACTAAAAC | | |
| | | | (SEQ ID. NO: 993) | (SEQ ID. | (SEQ ID. | | |
| | | | | NO: 994) | NO: 995) | | |
| 1b | zika_13 | LwaCas13a | GATTTAGACTACCCCAAAA | accagtgctt | GATTTAGACTAC | Zika | 1b |
| | | | ACGAAGGGGACTAAAACacc | ctttgttgttc | CCCAAAAACGAA | ssRNA | |
| | | | agtgcttctttgttgttccagtgtg | cagtgtg | GGGGACTAAAAC | | |
| | | | (SEQ ID. NO: 996) | (SEQ ID. | (SEQ ID. | | |
| | | | | NO: 997) | NO: 998) | | |
| 1b | zika_14 | LwaCas13a | GATTTAGACTACCCCAAAA | taccagtgct | GATTTAGACTAC | Zika | 1b |
| | | | ACGAAGGGGACTAAAACtac | tctttgttgtt | CCCAAAAACGAA | ssRNA | |
| | | | cagtgcttctttgttgttccagtgt | ccagtgt | GGGGACTAAAAC | | |
| | | | (SEQ ID. NO: 999) | (SEQ ID. | (SEQ ID. | | |
| | | | | NO: | NO: 1001) | | |
| | | | | 1000) | | | |
| 1b | zika_15 | LwaCas13a | GATTTAGACTACCCCAAAA | ctaccagtgc | GATTTAGACTAC | Zika | 1b |
| | | | ACGAAGGGGACTAAAACcta | ttetttgttgt | CCCAAAAACGAA | ssRNA | |
| | | | ccagtgcttctttgttgttccagtg | tccagtg | GGGGACTAAAAC | | |
| | | | (SEQ ID. NO: 1002) | (SEQ ID. | (SEQ ID. | | |
| | | | | NO: | NO: 1004) | | |
| | | | | 1003) | | | |
| 1b | zika_16 | LwaCas13a | GATTTAGACTACCCCAAAA | tctaccagtg | GATTTAGACTAC | Zika | 1b |
| | | | ACGAAGGGGACTAAAACtct | cttctttgttg | CCCAAAAACGAA | ssRNA | |
| | | | accagtgcttctttgttgttccagt | ttccagt | GGGGACTAAAAC | | |
| | | | (SEQ ID. NO: 1005) | (SEQ ID. | (SEQ ID. | | |
| | | | | NO: | NO: 1007) | | |
| | | | | 1006) | | | |
| 1b | zika_17 | LwaCas13a | GATTTAGACTACCCCAAAA | ctctaccagt | GATTTAGACTAC | Zika | 1b |
| | | | ACGAAGGGGACTAAAACctc | gcttctttgtt | CCCAAAAACGAA | ssRNA | |
| | | | taccagtgcttctttgttgttccag | gttccag | GGGGACTAAAAC | | |
| | | | (SEQ ID. NO: 1008) | (SEQ ID. | (SEQ ID. | | |
| | | | | NO: | NO: 1010) | | |
| | | | | 1009) | | | |
| 1b | zika_18 | LwaCas13a | GATTTAGACTACCCCAAAA | actctaccag | GATTTAGACTAC | Zika | 1b |
| | | | ACGAAGGGGACTAAAACact | tgcttctttgt | CCCAAAAACGAA | ssRNA | |
| | | | ctaccagtgcttctttgttgttcca | tgttcca | GGGGACTAAAAC | | |
| | | | (SEQ ID. NO: 1011) | (SEQ ID. | (SEQ ID. | | |
| | | | | NO: | NO: 1013) | | |
| | | | | 1012) | | | |
| 1b | zika_19 | LwaCas13a | GATTTAGACTACCCCAAAA | aactctacca | GATTTAGACTAC | Zika | 1b |
| | | | ACGAAGGGGACTAAAACaac | gtgcttctttg | CCCAAAAACGAA | ssRNA | |
| | | | tctaccagtgcttctttgttgttcc | ttgttcc | GGGGACTAAAAC | | |
| | | | (SEQ ID. NO: 1014) | (SEQ ID. | (SEQ ID. | | |
| | | | | NO: | NO: 1016) | | |
| | | | | 1015) | | | |
| 1b | zika_20 | LwaCas13a | GATTTAGACTACCCCAAAA | tgaactctac | GATTTAGACTAC | Zika | 1b |
| | | | ACGAAGGGGACTAAAACtga | cagtgcttctt | CCCAAAAACGAA | ssRNA | |
| | | | actctaccagtgcttctttgttgtt | tgttgtt | GGGGACTAAAAC | | |
| | | | (SEQ ID. NO: 1017) | (SEQ ID. | (SEQ ID. | | |
| | | | | NO: | NO: 1019) | | |
| | | | | 1018) | | | |
| 1b | zika_21 | LwaCas13a | GATTTAGACTACCCCAAAA | cttgaactct | GATTTAGACTAC | Zika | 1b |
| | | | ACGAAGGGGACTAAAACctt | accagtgctt | CCCAAAAACGAA | ssRNA | |
| | | | gaactctaccagtgcttctttgttg | ctttgttg | GGGGACTAAAAC | | |
| | | | (SEQ ID. NO: 1020) | (SEQ ID. | (SEQ ID. | | |
| | | | | NO: | NO: 1022) | | |
| | | | | 1021) | | | |
| 1b | zika_22 | LwaCas13a | GATTTAGACTACCCCAAAA | tccttgaact | GATTTAGACTAC | Zika | 1b |
| | | | ACGAAGGGGACTAAAACtcc | ctaccagtgc | CCCAAAAACGAA | ssRNA | |
| | | | ttgaactctaccagtgcttctttgt | ttctttgt | GGGGACTAAAAC | | |
| | | | (SEQ ID. NO: 1023) | (SEQ ID. | (SEQ ID. | | |
| | | | | NO: | NO: 1025) | | |
| | | | | 1024) | | | |
| 1b | zika_23 | LwaCas13a | GATTTAGACTACCCCAAAA | cgtccttgaa | GATTTAGACTAC | Zika | 1b |
| | | | ACGAAGGGGACTAAAACcgt | ctctaccagt | CCCAAAAACGAA | ssRNA | |
| | | | ccttgaactctaccagtgcttcttt | gcttcttt | GGGGACTAAAAC | | |
| | | | (SEQ ID. NO: 1026) | (SEQ ID. | (SEQ ID. | | |
| | | | | NO: | NO: 1028) | | |
| | | | | 1027) | | | |
| 1b | zika_24 | LwaCas13a | GATTTAGACTACCCCAAAA | tgcgtccttg | GATTTAGACTAC | Zika | 1b |
| | | | ACGAAGGGGACTAAAACtgc | aactctacca | CCCAAAAACGAA | ssRNA | |
| | | | gtccttgaactctaccagtgcttct | gtgcttct | GGGGACTAAAAC | | |
| | | | (SEQ ID. NO: 1029) | (SEQ ID. | (SEQ ID. | | |
| | | | | NO: | NO: 1031) | | |
| | | | | 1030) | | | |
| 1b | zika_25 | LwaCas13a | GATTTAGACTACCCCAAAA | tgtgegtcctt | GATTTAGACTAC | Zika | 1b |
| | | | ACGAAGGGGACTAAAACtgt | gaactctacc | CCCAAAAACGAA | ssRNA | |
| | | | gegtccttgaactctaccagtgctt | agtgctt | GGGGACTAAAAC | | |
| | | | (SEQ ID. NO: 1032) | (SEQ ID. | (SEQ ID. | | |
| | | | | NO: | NO: 1034) | | |
| | | | | 1033) | | | |
| 1b | zika_26 | LwaCas13a | GATTTAGACTACCCCAAAA | catgtgcgtc | GATTTAGACTAC | Zika | 1b |
| | | | ACGAAGGGGACTAAAACcat | cttgaactct | CCCAAAAACGAA | ssRNA | |
| | | | gtgcgtccttgaactctaccagtgc | accagtgc | GGGGACTAAAAC | | |
| | | | (SEQ ID. NO: 1035) | (SEQ ID. | (SEQ ID. | | |
| | | | | NO: | NO: 1037) | | |
| | | | | 1036) | | | |
| 1b | zika_27 | LwaCas13a | GATTTAGACTACCCCAAAA | ggcatgtgc | GATTTAGACTAC | Zika | 1b |
| | | | ACGAAGGGGACTAAAACggc | gtccttgaac | CCCAAAAACGAA | ssRNA | |
| | | | atgtgcgtccttgaactctaccagt | tctaccagt | GGGGACTAAAAC | | |
| | | | (SEQ ID. NO: 1038) | (SEQ ID. | (SEQ ID. | | |
| | | | | NO: | NO: 1040) | | |
| | | | | 1039) | | | |
| 1b | zika_28 | LwaCas13a | GATTTAGACTACCCCAAAA | ttggcatgtg | GATTTAGACTAC | Zika | 1b |
| | | | ACGAAGGGGACTAAAACttg | cgtccttgaa | CCCAAAAACGAA | ssRNA | |
| | | | gcatgtgcgtccttgaactctacca | ctctacca | GGGGACTAAAAC | | |
| | | | (SEQ ID. NO: 1041) | (SEQ ID. | (SEQ ID. | | |
| | | | | NO: | NO: 1043) | | |
| | | | | 1042) | | | |
| 1b | zika_29 | LwaCas13a | GATTTAGACTACCCCAAAA | ttttggcatgt | GATTTAGACTAC | Zika | 1b |
| | | | ACGAAGGGGACTAAAACtttt | gcgtccttga | CCCAAAAACGAA | ssRNA | |
| | | | ggcatgtgcgtccttgaactctac | actctac | GGGGACTAAAAC | | |
| | | | (SEQ ID. NO: 1044) | (SEQ ID. | (SEQ ID. | | |
| | | | | NO: | NO: 1046) | | |
| | | | | 1045) | | | |
| 1b | zika_30 | LwaCas13a | GATTTAGACTACCCCAAAA | ccttttggcat | GATTTAGACTAC | Zika | 1b |
| | | | ACGAAGGGGACTAAAACcct | gtgcgtcctt | CCCAAAAACGAA | ssRNA | |
| | | | tttggcatgtgegtccttgaactct | gaactct | GGGGACTAAAAC | | |
| | | | (SEQ ID. NO: 1047) | (SEQ ID. | (SEQ ID. | | |
| | | | | NO: | NO: 1049) | | |
| | | | | 1048) | | | |
| 1b | zika_31 | LwaCas13a | GATTTAGACTACCCCAAAA | tgccttttggc | GATTTAGACTAC | Zika | 1b |
| | | | ACGAAGGGGACTAAAACtgc | atgtgcgtcc | CCCAAAAACGAA | ssRNA | |
| | | | cttttggcatgtgcgtccttgaact | ttgaact | GGGGACTAAAAC | | |
| | | | (SEQ ID. NO: 1050) | (SEQ ID. | (SEQ ID. | | |
| | | | | NO: | NO: 1052) | | |
| | | | | 1051) | | | |
| 1b | zika_32 | LwaCas13a | GATTTAGACTACCCCAAAA | tttgccttttg | GATTTAGACTAC | Zika | 1b |
| | | | ACGAAGGGGACTAAAACttt | gcatgtgcgt | CCCAAAAACGAA | ssRNA | |
| | | | gccttttggcatgtgcgtccttgaa | ccttgaa | GGGGACTAAAAC | | |
| | | | (SEQ ID. NO: 1053) | (SEQ ID. | (SEQ ID. | | |
| | | | | NO: | NO: 1055) | | |
| | | | | 1054) | | | |
| 1b | zika_33 | LwaCas13a | GATTTAGACTACCCCAAAA | agtttgccttt | GATTTAGACTAC | Zika | 1b |
| | | | ACGAAGGGGACTAAAACagt | tggcatgtgc | CCCAAAAACGAA | ssRNA | |
| | | | ttgccttttggcatgtgcgtccttg | gtccttg | GGGGACTAAAAC | | |
| | | | (SEQ ID. NO: 1056) | (SEQ ID. | (SEQ ID. | | |
| | | | | NO: | NO: 1058) | | |
| | | | | 1057) | | | |
| 1b | zika_34 | LwaCas13a | GATTTAGACTACCCCAAAA | acagtttgcc | GATTTAGACTAC | Zika | 1b |
| | | | ACGAAGGGGACTAAAACaca | ttttggcatgt | CCCAAAAACGAA | ssRNA | |
| | | | gtttgccttttggcatgtgcgtcct | gcgtcct | GGGGACTAAAAC | | |
| | | | (SEQ ID. NO: 1059) | (SEQ ID. | (SEQ ID. | | |
| | | | | NO: | NO: 1061) | | |
| | | | | 1060) | | | |
| 1b | zika_35 | LwaCas13a | GATTTAGACTACCCCAAAA | cgacagtttg | GATTTAGACTAC | Zika | 1b |
| | | | ACGAAGGGGACTAAAACcga | ccttttggcat | CCCAAAAACGAA | ssRNA | |
| | | | cagtttgccttttggcatgtgcgtc | gtgcgtc | GGGGACTAAAAC | | |
| | | | (SEQ ID. NO: 1062) | (SEQ ID. | (SEQ ID. | | |
| | | | | NO: | NO: 1064) | | |
| | | | | 1063) | | | |
| 1b | zika_36 | LwaCas13a | GATTTAGACTACCCCAAAA | cacgacagtt | GATTTAGACTAC | Zika | 1b |
| | | | ACGAAGGGGACTAAAACcac | tgccttttggc | CCCAAAAACGAA | ssRNA | |
| | | | gacagtttgccttttggcatgtgcg | atgtgcg | GGGGACTAAAAC | | |
| | | | (SEQ ID. NO: 1065) | (SEQ ID. | (SEQ ID. | | |
| | | | | NO: | NO: 1067) | | |
| | | | | 1066) | | | |
| 1b | zika_37 | LwaCas13a | GATTTAGACTACCCCAAAA | accacgaca | GATTTAGACTAC | Zika | 1b |
| | | | ACGAAGGGGACTAAAACacc | gtttgcctttt | CCCAAAAACGAA | ssRNA | |
| | | | acgacagtttgccttttggcatgtg | ggcatgtg | GGGGACTAAAAC | | |
| | | | (SEQ ID. NO: 1068) | (SEQ ID. | (SEQ ID. | | |
| | | | | NO: | NO: 1070) | | |
| | | | | 1069) | | | |
| 1b | zika_38 | LwaCas13a | GATTTAGACTACCCCAAAA | gaaccacga | GATTTAGACTAC | Zika | 1b |
| | | | ACGAAGGGGACTAAAACgaa | cagtttgcctt | CCCAAAAACGAA | ssRNA | |
| | | | ccacgacagtttgccttttggcatg | ttggcatg | GGGGACTAAAAC | | |
| | | | (SEQ ID. NO: 1071) | (SEQ ID. | (SEQ ID. | | |
| | | | | NO: | NO: 1073) | | |
| | | | | 1072) | | | |
| 1b | zika_39 | LwaCas13a | GATTTAGACTACCCCAAAA | tagaaccac | GATTTAGACTAC | Zika | 1b |
| | | | ACGAAGGGGACTAAAACtag | gacagtttgc | CCCAAAAACGAA | ssRNA | |
| | | | aaccacgacagtttgccttttggca | cttttggca | GGGGACTAAAAC | | |
| | | | (SEQ ID. NO: 1074) | (SEQ ID. | (SEQ ID. | | |
| | | | | NO: | NO: 1076) | | |
| | | | | 1075) | | | |
| 1b | zika_40 | LwaCas13a | GATTTAGACTACCCCAAAA | cctagaacc | GATTTAGACTAC | Zika | 1b |
| | | | ACGAAGGGGACTAAAACcct | acgacagttt | CCCAAAAACGAA | ssRNA | |
| | | | agaaccacgacagtttgccttttgg | gccttttgg | GGGGACTAAAAC | | |
| | | | (SEQ ID. NO: 1077) | (SEQ ID. | (SEQ ID. | | |
| | | | | NO: | NO: 1079) | | |
| | | | | 1078) | | | |
| 1b | zika_41 | LwaCas13a | GATTTAGACTACCCCAAAA | tccctagaac | GATTTAGACTAC | Zika | 1b |
| | | | ACGAAGGGGACTAAAACtcc | cacgacagtt | CCCAAAAACGAA | ssRNA | |
| | | | ctagaaccacgacagtttgcctttt | tgcctttt | GGGGACTAAAAC | | |
| | | | (SEQ ID. NO: 1080) | (SEQ ID. | (SEQ ID. | | |
| | | | | NO: | NO: 1082) | | |
| | | | | 1081) | | | |
| 1b | zika_42 | LwaCas13a | GATTTAGACTACCCCAAAA | actccctaga | GATTTAGACTAC | Zika | 1b |
| | | | ACGAAGGGGACTAAAACact | accacgaca | CCCAAAAACGAA | ssRNA | |
| | | | ccctagaaccacgacagtttgcctt | gtttgcctt | GGGGACTAAAAC | | |
| | | | (SEQ ID. NO: 1083) | (SEQ ID. | (SEQ ID. | | |
| | | | | NO: | NO: 1085) | | |
| | | | | 1084) | | | |
| 1b | zika_43 | LwaCas13a | GATTTAGACTACCCCAAAA | tgactcccta | GATTTAGACTAC | Zika | 1b |
| | | | ACGAAGGGGACTAAAACtga | gaaccacga | CCCAAAAACGAA | ssRNA | |
| | | | ctccctagaaccacgacagtttgcc | cagtttgcc | GGGGACTAAAAC | | |
| | | | (SEQ ID. NO: 1086) | (SEQ ID. | (SEQ ID. | | |
| | | | | NO: | NO: 1088) | | |
| | | | | 1087) | | | |
| 1b | zika_44 | LwaCas13a | GATTTAGACTACCCCAAAA | cttgactccc | GATTTAGACTAC | Zika | 1b |
| | | | ACGAAGGGGACTAAAACctt | tagaaccac | CCCAAAAACGAA | ssRNA | |
| | | | gactccctagaaccacgacagtttg | gacagtttg | GGGGACTAAAAC | | |
| | | | (SEQ ID. NO: 1089) | (SEQ ID. | (SEQ ID. | | |
| | | | | NO: | NO: 1091) | | |
| | | | | 1090) | | | |
| 1b | zika_45 | LwaCas13a | GATTTAGACTACCCCAAAA | ttcttgactcc | GATTTAGACTAC | Zika | 1b |
| | | | ACGAAGGGGACTAAAACttc | ctagaacca | CCCAAAAACGAA | ssRNA | |
| | | | ttgactccctagaaccacgacagtt | cgacagtt | GGGGACTAAAAC | | |
| | | | (SEQ ID. NO: 1092) | (SEQ ID. | (SEQ ID. | | |
| | | | | NO: | NO: 1094) | | |
| | | | | 1093) | | | |
| 1b | zika_46 | LwaCas13a | GATTTAGACTACCCCAAAA | ccttcttgact | GATTTAGACTAC | Zika | 1b |
| | | | ACGAAGGGGACTAAAACcct | ccctagaac | CCCAAAAACGAA | ssRNA | |
| | | | tcttgactccctagaaccacgacag | cacgacag | GGGGACTAAAAC | | |
| | | | (SEQ ID. NO: 1095) | (SEQ ID. | (SEQ ID. | | |
| | | | | NO: | NO: 1097) | | |
| | | | | 1096) | | | |
| 1b | zika_47 | LwaCas13a | GATTTAGACTACCCCAAAA | ctccttcttga | GATTTAGACTAC | Zika | 1b |
| | | | ACGAAGGGGACTAAAACctc | ctccctagaa | CCCAAAAACGAA | ssRNA | |
| | | | cttcttgactccctagaaccacgac | ccacgac | GGGGACTAAAAC | | |
| | | | (SEQ ID. NO: 1098) | (SEQ ID. | (SEQ ID. | | |
| | | | | NO: | NO: 1100) | | |
| | | | | 1099) | | | |
| 1b | zika_48 | LwaCas13a | GATTTAGACTACCCCAAAA | tgctccttctt | GATTTAGACTAC | Zika | 1b |
| | | | ACGAAGGGGACTAAAACtgc | gactccctag | CCCAAAAACGAA | ssRNA | |
| | | | tecttcttgactccctagaaccacg | aaccacg | GGGGACTAAAAC | | |
| | | | (SEQ ID. NO: 1101) | (SEQ ID. | (SEQ ID. | | |
| | | | | NO: | NO: 1103) | | |
| | | | | 1102) | | | |
| 1b | zika_49 | LwaCas13a | GATTTAGACTACCCCAAAA | actgctcctt | GATTTAGACTAC | Zika | 1b |
| | | | ACGAAGGGGACTAAAACact | cttgactccc | CCCAAAAACGAA | ssRNA | |
| | | | gctccttcttgactccctagaacca | tagaacca | GGGGACTAAAAC | | |
| | | | (SEQ ID. NO: 1104) | (SEQ ID. | (SEQ ID. | | |
| | | | | NO: | NO: 1106) | | |
| | | | | 1105) | | | |
| 1b | zika_50 | LwaCas13a | GATTTAGACTACCCCAAAA | gaactgctcc | GATTTAGACTAC | Zika | 1b |
| | | | ACGAAGGGGACTAAAACgaa | ttcttgactcc | CCCAAAAACGAA | ssRNA | |
| | | | ctgctccttcttgactccctagaac | ctagaac | GGGGACTAAAAC | | |
| | | | (SEQ ID. NO: 1107) | (SEQ ID. | (SEQ ID. | | |
| | | | | NO: | NO: 1109) | | |
| | | | | 1108) | | | |
| 1b | zika_51 | LwaCas13a | GATTTAGACTACCCCAAAA | gtgaactgct | GATTTAGACTAC | Zika | 1b |
| | | | ACGAAGGGGACTAAAACgtg | ccttcttgact | CCCAAAAACGAA | ssRNA | |
| | | | aactgctccttcttgactccctaga | ccctaga | GGGGACTAAAAC | | |
| | | | (SEQ ID. NO: 1110) | (SEQ ID. | (SEQ ID. | | |
| | | | | NO: | NO: 1112) | | |
| | | | | 1111) | | | |
| 1b | zika_52 | LwaCas13a | GATTTAGACTACCCCAAAA | gtgtgaactg | GATTTAGACTAC | Zika | 1b |
| | | | ACGAAGGGGACTAAAACgtg | ctccttcttga | CCCAAAAACGAA | ssRNA | |
| | | | tgaactgctccttcttgactcccta | ctcccta | GGGGACTAAAAC | | |
| | | | (SEQ ID. NO: 1113) | (SEQ ID. | (SEQ ID. | | |
| | | | | NO: | NO: 1115) | | |
| | | | | 1114) | | | |
| 1b | zika_53 | LwaCas13a | GATTTAGACTACCCCAAAA | ccgtgtgaa | GATTTAGACTAC | Zika | 1b |
| | | | ACGAAGGGGACTAAAACccg | ctgctccttct | CCCAAAAACGAA | ssRNA | |
| | | | tgtgaactgctccttcttgactccc | tgactccc | GGGGACTAAAAC | | |
| | | | (SEQ ID. NO: 1116) | (SEQ ID. | (SEQ ID. | | |
| | | | | NO: | NO: 1118) | | |
| | | | | 1117) | | | |
| 1b | zika_54 | LwaCas13a | GATTTAGACTACCCCAAAA | ggccgtgtg | GATTTAGACTAC | Zika | 1b |
| | | | ACGAAGGGGACTAAAACggc | aactgctcct | CCCAAAAACGAA | ssRNA | |
| | | | cgtgtgaactgctccttcttgactc | tcttgactc | GGGGACTAAAAC | | |
| | | | (SEQ ID. NO: 1119) | (SEQ ID. | (SEQ ID. | | |
| | | | | NO: | NO: 1121) | | |
| | | | | 1120) | | | |
| 1b | zika_55 | LwaCasl3a | GATTTAGACTACCCCAAAA | agggccgtg | GATTTAGACTAC | Zika | 1b |
| | | | ACGAAGGGGACTAAAACagg | tgaactgctc | CCCAAAAACGAA | ssRNA | |
| | | | gccgtgtgaactgctccttcttgac | cttcttgac | GGGGACTAAAAC | | |
| | | | (SEQ ID. NO: 1122) | (SEQ ID. | (SEQ ID. | | |
| | | | | NO: | NO: 1124) | | |
| | | | | 1123) | | | |
| 1b | zika_56 | LwaCasl3a | GATTTAGACTACCCCAAAA | caagggccg | GATTTAGACTAC | Zika | 1b |
| | | | ACGAAGGGGACTAAAACcaa | tgtgaactgc | CCCAAAAACGAA | ssRNA | |
| | | | gggccgtgtgaactgctccttcttg | tccttcttg | GGGGACTAAAAC | | |
| | | | (SEQ ID. NO: 1125) | (SEQ ID. | (SEQ ID. | | |
| | | | | NO: | NO: 1127) | | |
| | | | | 1126) | | | |
| 1b | zika_57 | LwaCasl3a | GATTTAGACTACCCCAAAA | agcaagggc | GATTTAGACTAC | Zika | 1b |
| | | | ACGAAGGGGACTAAAACagc | cgtgtgaact | CCCAAAAACGAA | ssRNA | |
| | | | aagggccgtgtgaactgctccttct | gctccttct | GGGGACTAAAAC | | |
| | | | (SEQ ID. NO: 1128) | (SEQ ID. | (SEQ ID. | | |
| | | | | NO: | NO: 1130) | | |
| | | | | 1129) | | | |
| 1b | zika_58 | LwaCasl3a | GATTTAGACTACCCCAAAA | ccagcaagg | GATTTAGACTAC | Zika | 1b |
| | | | ACGAAGGGGACTAAAACcca | gccgtgtga | CCCAAAAACGAA | ssRNA | |
| | | | gcaagggccgtgtgaactgctcctt | actgctcctt | GGGGACTAAAAC | | |
| | | | (SEQ ID. NO: 1131) | (SEQ ID. | (SEQ ID. | | |
| | | | | NO: | NO: 1133) | | |
| | | | | 1132) | | | |
| 1b | zika_59 | LwaCasl3a | GATTTAGACTACCCCAAAA | ctccagcaa | GATTTAGACTAC | Zika | 1b |
| | | | ACGAAGGGGACTAAAACctc | gggccgtgt | CCCAAAAACGAA | ssRNA | |
| | | | cagcaagggccgtgtgaactgctcc | gaactgctcc | GGGGACTAAAAC | | |
| | | | (SEQ ID. NO: 1134) | (SEQ ID. | (SEQ ID. | | |
| | | | | NO: | NO: 1136) | | |
| | | | | 1135) | | | |
| 1b | zika_60 | LwaCasl3a | GATTTAGACTACCCCAAAA | agctccagc | GATTTAGACTAC | Zika | 1b |
| | | | ACGAAGGGGACTAAAACagc | aagggccgt | CCCAAAAACGAA | ssRNA | |
| | | | tccagcaagggccgtgtgaactgct | gtgaactgct | GGGGACTAAAAC | | |
| | | | (SEQ ID. NO: 1137) | (SEQ ID. | (SEQ ID. | | |
| | | | | NO: | NO: 1139) | | |
| | | | | 1138) | | | |
| 1b | zika_61 | LwaCasl3a | GATTTAGACTACCCCAAAA | agagctcca | GATTTAGACTAC | Zika | 1b |
| | | | ACGAAGGGGACTAAAACaga | gcaagggcc | CCCAAAAACGAA | ssRNA | |
| | | | gctccagcaagggccgtgtgaactg | gtgtgaactg | GGGGACTAAAAC | | |
| | | | (SEQ ID. NO: 1140) | (SEQ ID. | (SEQ ID. | | |
| | | | | NO: | NO: 1142) | | |
| | | | | 1141) | | | |
| 1b | zika_62 | LwaCasl3a | GATTTAGACTACCCCAAAA | ccagagctc | GATTTAGACTAC | Zika | 1b |
| | | | ACGAAGGGGACTAAAACcca | cagcaaggg | CCCAAAAACGAA | ssRNA | |
| | | | gagctccagcaagggccgtgtgaac | ccgtgtgaa | GGGGACTAAAAC | | |
| | | | (SEQ ID. NO: 1143) | c (SEQ | (SEQ ID. | | |
| | | | | ID. NO: | NO: 1145) | | |
| | | | | 1144) | | | |
| 1b | zika_63 | LwaCasl3a | GATTTAGACTACCCCAAAA | ctccagagct | GATTTAGACTAC | Zika | 1b |
| | | | ACGAAGGGGACTAAAACctc | ccagcaagg | CCCAAAAACGAA | ssRNA | |
| | | | cagagctccagcaagggccgtgtga | gccgtgtga | GGGGACTAAAAC | | |
| | | | (SEQ ID. NO: 1146) | (SEQ ID. | (SEQ ID. | | |
| | | | | NO: | NO: 1148) | | |
| | | | | 1147) | | | |
| 1b | zika_64 | LwaCas13a | GATTTAGACTACCCCAAAA | gcctccaga | GATTTAGACTAC | Zika | 1b |
| | | | ACGAAGGGGACTAAAACgcc | gctccagca | CCCAAAAACGAA | ssRNA | |
| | | | tccagagctccagcaagggccgtgt | agggccgtg | GGGGACTAAAAC | | |
| | | | (SEQ ID. NO: 1149) | t (SEQ | (SEQ ID. | | |
| | | | | ID. NO: | NO: 1151) | | |
| | | | | 1150) | | | |
| 1b | zika_65 | LwaCas13a | GATTTAGACTACCCCAAAA | cagcctcca | GATTTAGACTAC | Zika | 1b |
| | | | ACGAAGGGGACTAAAACcag | gagctccag | CCCAAAAACGAA | ssRNA | |
| | | | cctccagagctccagcaagggccgt | caagggccg | GGGGACTAAAAC | | |
| | | | (SEQ ID. NO: 1152) | t (SEQ | (SEQ ID. | | |
| | | | | ID. NO: | NO: 1154) | | |
| | | | | 1153) | | | |
| 1b | zika_66 | LwaCas13a | GATTTAGACTACCCCAAAA | ctcagcctcc | GATTTAGACTAC | Zika | 1b |
| | | | ACGAAGGGGACTAAAACctc | agagctcca | CCCAAAAACGAA | ssRNA | |
| | | | agcctccagagctccagcaagggcc | gcaagggcc | GGGGACTAAAAC | | |
| | | | (SEQ ID. NO: 1155) | (SEQ ID. | (SEQ ID. | | |
| | | | | NO: | NO: 1157) | | |
| | | | | 1156) | | | |
| 1b | zika_67 | LwaCas13a | GATTTAGACTACCCCAAAA | atctcagcct | GATTTAGACTAC | Zika | 1b |
| | | | ACGAAGGGGACTAAAACatc | ccagagctc | CCCAAAAACGAA | ssRNA | |
| | | | tcagcctccagagctccagcaaggg | cagcaaggg | GGGGACTAAAAC | | |
| | | | (SEQ ID. NO: 1158) | (SEQ ID. | (SEQ ID. | | |
| | | | | NO: | NO: 1160) | | |
| | | | | 1159) | | | |
| 1b | zika_68 | LwaCas13a | GATTTAGACTACCCCAAAA | catctcagcc | GATTTAGACTAC | Zika | 1b |
| | | | ACGAAGGGGACTAAAACcat | tccagagctc | CCCAAAAACGAA | ssRNA | |
| | | | ctcagcctccagagctccagcaagg | cagcaagg | GGGGACTAAAAC | | |
| | | | (SEQ ID. NO: 1161) | (SEQ ID. | (SEQ ID. | | |
| | | | | NO: | NO: 1163) | | |
| | | | | 1162) | | | |
| 1b | zika_69 | LwaCas13a | GATTTAGACTACCCCAAAA | ccatctcagc | GATTTAGACTAC | Zika | 1b |
| | | | ACGAAGGGGACTAAAACcca | ctccagagct | CCCAAAAACGAA | ssRNA | |
| | | | tctcagcctccagagctccagcaag | ccagcaag | GGGGACTAAAAC | | |
| | | | (SEQ ID. NO: 1164) | (SEQ ID. | (SEQ ID. | | |
| | | | | NO: | NO: 1166) | | |
| | | | | 1165) | | | |
| 1b | zika_70 | LwaCas13a | GATTTAGACTACCCCAAAA | tccatctcag | GATTTAGACTAC | Zika | 1b |
| | | | ACGAAGGGGACTAAAACtcc | cctccagag | CCCAAAAACGAA | ssRNA | |
| | | | atctcagcctccagagctccagcaa | ctccagcaa | GGGGACTAAAAC | | |
| | | | (SEQ ID. NO: 1167) | (SEQ ID. | (SEQ ID. | | |
| | | | | NO: | NO: 1169) | | |
| | | | | 1168) | | | |
| 1b | zika_71 | LwaCas13a | GATTTAGACTACCCCAAAA | atccatctca | GATTTAGACTAC | Zika | 1b |
| | | | ACGAAGGGGACTAAAACatc | gcctccaga | CCCAAAAACGAA | ssRNA | |
| | | | catctcagcctccagagctccagca | gctccagca | GGGGACTAAAAC | | |
| | | | (SEQ ID. NO: 1170) | (SEQ ID. | (SEQ ID. | | |
| | | | | NO: | NO: 1172) | | |
| | | | | 1171) | | | |
| 1b | zika_72 | LwaCas13a | GATTTAGACTACCCCAAAA | catccatctc | GATTTAGACTAC | Zika | 1b |
| | | | ACGAAGGGGACTAAAACcat | agcctccag | CCCAAAAACGAA | ssRNA | |
| | | | ccatctcagcctccagagctccagc | agctccagc | GGGGACTAAAAC | | |
| | | | (SEQ ID. NO: 1173) | (SEQ ID. | (SEQ ID. | | |
| | | | | NO: | NO: 1175) | | |
| | | | | 1174) | | | |
| 1b | zika_73 | LwaCas13a | GATTTAGACTACCCCAAAA | ccatccatct | GATTTAGACTAC | Zika | 1b |
| | | | ACGAAGGGGACTAAAACcca | cagcctcca | CCCAAAAACGAA | ssRNA | |
| | | | tccatctcagcctccagagctccag | gagctccag | GGGGACTAAAAC | | |
| | | | (SEQ ID. NO: 1176) | (SEQ ID. | (SEQ ID. | | |
| | | | | NO: | NO: 1178) | | |
| | | | | 1177) | | | |
| 1b | zika_74 | LwaCas13a | GATTTAGACTACCCCAAAA | accatccatc | GATTTAGACTAC | Zika | 1b |
| | | | ACGAAGGGGACTAAAACacc | tcagcctcca | CCCAAAAACGAA | ssRNA | |
| | | | atccatctcagcctccagagctcca | gagctcca | GGGGACTAAAAC | | |
| | | | (SEQ ID. NO: 1179) | (SEQ ID. | (SEQ ID. | | |
| | | | | NO: | NO: 1181) | | |
| | | | | 1180) | | | |
| 1b | zika_75 | LwaCas13a | GATTTAGACTACCCCAAAA | caccatccat | GATTTAGACTAC | Zika | 1b |
| | | | ACGAAGGGGACTAAAACcac | ctcagcctcc | CCCAAAAACGAA | ssRNA | |
| | | | catccatctcagcctccagagctcc | agagctcc | GGGGACTAAAAC | | |
| | | | (SEQ ID. NO: 1182) | (SEQ ID. | (SEQ ID. | | |
| | | | | NO: | NO: 1184) | | |
| | | | | 1183) | | | |
| 1b | zika_76 | LwaCas13a | GATTTAGACTACCCCAAAA | gcaccatcc | GATTTAGACTAC | Zika | 1b |
| | | | ACGAAGGGGACTAAAACgca | atctcagcct | CCCAAAAACGAA | ssRNA | |
| | | | ccatccatctcagcctccagagctc | ccagagctc | GGGGACTAAAAC | | |
| | | | (SEQ ID. NO: 1185) | (SEQ ID. | (SEQ ID. | | |
| | | | | NO: | NO: 1187) | | |
| | | | | 1186) | | | |
| 1b | zika_77 | LwaCas13a | GATTTAGACTACCCCAAAA | tgcaccatcc | GATTTAGACTAC | Zika | 1b |
| | | | ACGAAGGGGACTAAAACtgc | atctcagcct | CCCAAAAACGAA | ssRNA | |
| | | | accatccatctcagcctccagagct | ccagagct | GGGGACTAAAAC | | |
| | | | (SEQ ID. NO: 1188) | (SEQ ID. | (SEQ ID. | | |
| | | | | NO: | NO: 1190) | | |
| | | | | 1189) | | | |
| 1b | zika_78 | LwaCas13a | GATTTAGACTACCCCAAAA | ttgcaccatc | GATTTAGACTAC | Zika | 1b |
| | | | ACGAAGGGGACTAAAACttg | catctcagcc | CCCAAAAACGAA | ssRNA | |
| | | | caccatccatctcagcctccagagc | tccagagc | GGGGACTAAAAC | | |
| | | | (SEQ ID. NO: 1191) | (SEQ ID. | (SEQ ID. | | |
| | | | | NO: | NO: 1193) | | |
| | | | | 1192) | | | |
| 1b | zika_79 | LwaCas13a | GATTTAGACTACCCCAAAA | tttgcaccat | GATTTAGACTAC | Zika | 1b |
| | | | ACGAAGGGGACTAAAACttt | ccatctcagc | CCCAAAAACGAA | ssRNA | |
| | | | gcaccatccatctcagcctccagag | ctccagag | GGGGACTAAAAC | | |
| | | | (SEQ ID. NO: 1194) | (SEQ ID. | (SEQ ID. | | |
| | | | | NO: | NO: 1196) | | |
| | | | | 1195) | | | |
| 1b | zika_80 | LwaCas13a | GATTTAGACTACCCCAAAA | ctttgcacca | GATTTAGACTAC | Zika | 1b |
| | | | ACGAAGGGGACTAAAACctt | tccatctcag | CCCAAAAACGAA | ssRNA | |
| | | | tgcaccatccatctcagcctccaga | cctccaga | GGGGACTAAAAC | | |
| | | | (SEQ ID. NO: 1197) | (SEQ ID. | (SEQ ID. | | |
| | | | | NO: | NO: 1199) | | |
| | | | | 1198) | | | |
| 1b | zika_81 | LwaCas13a | GATTTAGACTACCCCAAAA | cctttgcacc | GATTTAGACTAC | Zika | 1b |
| | | | ACGAAGGGGACTAAAACcct | atccatctca | CCCAAAAACGAA | ssRNA | |
| | | | ttgcaccatccatctcagcctccag | gcctccag | GGGGACTAAAAC | | |
| | | | (SEQ ID. NO: 1200) | (SEQ ID. | (SEQ ID. | | |
| | | | | NO: | NO: 1202) | | |
| | | | | 1201) | | | |
| 1b | zika_82 | LwaCas13a | GATTTAGACTACCCCAAAA | ccctttgcac | GATTTAGACTAC | Zika | 1b |
| | | | ACGAAGGGGACTAAAACccc | catccatctc | CCCAAAAACGAA | ssRNA | |
| | | | tttgcaccatccatctcagcctcca | agcctcca | GGGGACTAAAAC | | |
| | | | (SEQ ID. NO: 1203) | (SEQ ID. | (SEQ ID. | | |
| | | | | NO: | NO: 1205) | | |
| | | | | 1204) | | | |
| 1b | zika_83 | LwaCas13a | GATTTAGACTACCCCAAAA | tccctttgca | GATTTAGACTAC | Zika | 1b |
| | | | ACGAAGGGGACTAAAACtcc | ccatccatct | CCCAAAAACGAA | ssRNA | |
| | | | ctttgcaccatccatctcagcctcc | cagcctcc | GGGGACTAAAAC | | |
| | | | (SEQ ID. NO: 1206) | (SEQ ID. | (SEQ ID. | | |
| | | | | NO: | NO: 1208) | | |
| | | | | 1207) | | | |
| 1b | zika_84 | LwaCas13a | GATTTAGACTACCCCAAAA | ttccctttgca | GATTTAGACTAC | Zika | 1b |
| | | | ACGAAGGGGACTAAAACttc | ccatccatct | CCCAAAAACGAA | ssRNA | |
| | | | cctttgcaccatccatctcagcctc | cagcctc | GGGGACTAAAAC | | |
| | | | (SEQ ID. NO: 1209) | (SEQ ID. | (SEQ ID. | | |
| | | | | NO: | NO: 1211) | | |
| | | | | 1210) | | | |
| 1b | zika_85 | LwaCas13a | GATTTAGACTACCCCAAAA | cttccctttgc | GATTTAGACTAC | Zika | 1b |
| | | | ACGAAGGGGACTAAAACctt | accatccatc | CCCAAAAACGAA | ssRNA | |
| | | | ccctttgcaccatccatctcagcct | tcagcct | GGGGACTAAAAC | | |
| | | | (SEQ ID. NO: 1212) | (SEQ ID. | (SEQ ID. | | |
| | | | | NO: | NO: 1214) | | |
| | | | | 1213) | | | |
| 1b | zika_86 | LwaCas13a | GATTTAGACTACCCCAAAA | ccttccctttg | GATTTAGACTAC | Zika | 1b |
| | | | ACGAAGGGGACTAAAACcct | caccatccat | CCCAAAAACGAA | ssRNA | |
| | | | tecctttgcaccatccatctcagcc | ctcagcc | GGGGACTAAAAC | | |
| | | | (SEQ ID. NO: 1215) | (SEQ ID. | (SEQ ID. | | |
| | | | | NO: | NO: 1217) | | |
| | | | | 1216) | | | |
| 1b | zika_87 | LwaCas13a | GATTTAGACTACCCCAAAA | gccttcccttt | GATTTAGACTAC | Zika | 1b |
| | | | ACGAAGGGGACTAAAACgcc | gcaccatcc | CCCAAAAACGAA | ssRNA | |
| | | | ttccctttgcaccatccatctcagc | atctcagc | GGGGACTAAAAC | | |
| | | | (SEQ ID. NO: 1218) | (SEQ ID. | (SEQ ID. | | |
| | | | | NO: | NO: 1220) | | |
| | | | | 1219) | | | |
| 1b | zika_88 | LwaCas13a | GATTTAGACTACCCCAAAA | agccttccct | GATTTAGACTAC | Zika | 1b |
| | | | ACGAAGGGGACTAAAACagc | ttgcaccatc | CCCAAAAACGAA | ssRNA | |
| | | | cttccctttgcaccatccatctcag | catctcag | GGGGACTAAAAC | | |
| | | | (SEQ ID. NO: 1221) | (SEQ ID. | (SEQ ID. | | |
| | | | | NO: | NO: 1223) | | |
| | | | | 1222) | | | |
| 1b | zika_89 | LwaCas13a | GATTTAGACTACCCCAAAA | cagccttccc | GATTTAGACTAC | Zika | 1b |
| | | | ACGAAGGGGACTAAAACcag | tttgcaccat | CCCAAAAACGAA | ssRNA | |
| | | | ccttccctttgcaccatccatctca | ccatctca | GGGGACTAAAAC | | |
| | | | (SEQ ID. NO: 1224) | (SEQ ID. | (SEQ ID. | | |
| | | | | NO: | NO: 1226) | | |
| | | | | 1225) | | | |
| 1b | zika_90 | LwaCas13a | GATTTAGACTACCCCAAAA | acagccttcc | GATTTAGACTAC | Zika | 1b |
| | | | ACGAAGGGGACTAAAACaca | ctttgcacca | CCCAAAAACGAA | ssRNA | |
| | | | gccttccctttgcaccatccatctc | tccatctc | GGGGACTAAAAC | | |
| | | | (SEQ ID. NO: 1227) | (SEQ ID. | (SEQ ID. | | |
| | | | | NO: | NO: 1229) | | |
| | | | | 1228) | | | |
| 1b | zika_91 | LwaCas13a | GATTTAGACTACCCCAAAA | gacagccttc | GATTTAGACTAC | Zika | 1b |
| | | | ACGAAGGGGACTAAAACgac | cctttgcacc | CCCAAAAACGAA | ssRNA | |
| | | | agccttccctttgcaccatccatct | atccatct | GGGGACTAAAAC | | |
| | | | (SEQ ID. NO: 1230) | (SEQ ID. | (SEQ ID. | | |
| | | | | NO: | NO: 1232) | | |
| | | | | 1231) | | | |
| 1b | zika_92 | LwaCas13a | GATTTAGACTACCCCAAAA | ggacagcct | GATTTAGACTAC | Zika | 1b |
| | | | ACGAAGGGGACTAAAACgga | tccctttgca | CCCAAAAACGAA | ssRNA | |
| | | | cagccttccctttgcaccatccatc | ccatccatc | GGGGACTAAAAC | | |
| | | | (SEQ ID. NO: 1233) | (SEQ ID. | (SEQ ID. | | |
| | | | | NO: | NO: 1235) | | |
| | | | | 1234) | | | |
| 1b | zika_93 | LwaCas13a | GATTTAGACTACCCCAAAA | aggacagcc | GATTTAGACTAC | Zika | 1b |
| | | | ACGAAGGGGACTAAAACagg | ttccctttgca | CCCAAAAACGAA | ssRNA | |
| | | | acagccttccctttgcaccatccat | ccatccat | GGGGACTAAAAC | | |
| | | | (SEQ ID. NO: 1236) | (SEQ ID. | (SEQ ID. | | |
| | | | | NO: | NO: 1238) | | |
| | | | | 1237) | | | |
| 1b | zika_94 | LwaCas13a | GATTTAGACTACCCCAAAA | gaggacagc | GATTTAGACTAC | Zika | 1b |
| | | | ACGAAGGGGACTAAAACgag | cttccctttgc | CCCAAAAACGAA | ssRNA | |
| | | | gacagccttccctttgcaccatcca | accatcca | GGGGACTAAAAC | | |
| | | | (SEQ ID. NO: 1239) | (SEQ ID. | (SEQ ID. | | |
| | | | | NO: | NO: 1241) | | |
| | | | | 1240) | | | |
| 1b | zika_0 | CcaCas13b | tttgttgttccagtgtggagttccg | tttgttgttcc | GTTGGAACTGCT | Zika | 1b |
| | | | gtgtcGTTGGAACTGCTCTCATTTT | agtgtggagt | CTCATTTTGGAGG | ssRNA | |
| | | | GGAGGGTAATCACAAC | tccggtgtc | GTAATCACAAC | | |
| | | | (SEQ ID. NO: 1242) | (SEQ ID. | (SEQ ID. | | |
| | | | | NO: | NO: 1244) | | |
| | | | | 1243) | | | |
| 1b | zika_1 | CcaCas13b | ctttgttgttccagtgtggagttcc | ctttgttgttc | GTTGGAACTGCT | Zika | 1b |
| | | | ggtgtGTTGGAACTGCTCTCATTTT | cagtgtgga | CTCATTTTGGAGG | ssRNA | |
| | | | GGAGGGTAATCACAAC | gttccggtgt | GTAATCACAAC | | |
| | | | (SEQ ID. NO: 1245) | (SEQ ID. | (SEQ ID. | | |
| | | | | NO: | NO: 1247) | | |
| | | | | 1246) | | | |
| 1b | zika_2 | CcaCas13b | tctttgttgttccagtgtggagttc | tctttgttgtt | GTTGGAACTGCT | Zika | 1b |
| | | | cggtgGTTGGAACTGCTCTCATTTT | ccagtgtgga | CTCATTTTGGAGG | ssRNA | |
| | | | GGAGGGTAATCACAAC | gttccggtg | GTAATCACAAC | | |
| | | | (SEQ ID. NO: 1248) | (SEQ ID. | (SEQ ID. | | |
| | | | | NO: | NO: 1250) | | |
| | | | | 1249) | | | |
| 1b | zika_3 | CcaCas13b | ttctttgttgttccagtgtggagtt | ttctttgttgt | GTTGGAACTGCT | Zika | 1b |
| | | | ccggtGTTGGAACTGCTCTCATTTT | tccagtgtgg | CTCATTTTGGAGG | ssRNA | |
| | | | GGAGGGTAATCACAAC | agttccggt | GTAATCACAAC | | |
| | | | (SEQ ID. NO: 1251) | (SEQ ID. | (SEQ ID. | | |
| | | | | NO: | NO: 1253) | | |
| | | | | 1252) | | | |
| 1b | zika_4 | CcaCas13b | cttctttgttgttccagtgtggagt | cttctttgttg | GTTGGAACTGCT | Zika | 1b |
| | | | tccggGTTGGAACTGCTCTCATTTT | ttccagtgtgg | CTCATTTTGGAGG | ssRNA | |
| | | | GGAGGGTAATCACAAC | agttccgg | GTAATCACAAC | | |
| | | | (SEQ ID. NO: 1254) | (SEQ ID. | (SEQ ID. | | |
| | | | | NO: | NO: 1256) | | |
| | | | | 1255) | | | |
| 1b | zika_5 | CcaCas13b | gcttctttgttgttccagtgtggag | gcttctttgtt | GTTGGAACTGCT | Zika | 1b |
| | | | ttccgGTTGGAACTGCTCTCATTTT | gttccagtgt | CTCATTTTGGAGG | ssRNA | |
| | | | GGAGGGTAATCACAAC | ggagttccg | GTAATCACAAC | | |
| | | | (SEQ ID. NO: 1257) | (SEQ ID. | (SEQ ID. | | |
| | | | | NO: | NO: 1259) | | |
| | | | | 1258) | | | |
| 1b | zika_6 | CcaCas13b | tgcttctttgttgttccagtgtgga | tgcttctttgt | GTTGGAACTGCT | Zika | 1b |
| | | | gttccGTTGGAACTGCTCTCATTTT | tgttccagtgt | CTCATTTTGGAGG | ssRNA | |
| | | | GGAGGGTAATCACAAC | ggagttcc | GTAATCACAAC | | |
| | | | (SEQ ID. NO: 1260) | (SEQ ID. | (SEQ ID. | | |
| | | | | NO: | NO: 1262) | | |
| | | | | 1261) | | | |
| 1b | zika_7 | CcaCas13b | gtgcttctttgttgttccagtgtgg | gtgcttctttg | GTTGGAACTGCT | Zika | 1b |
| | | | agttcGTTGGAACTGCTCTCATTTT | ttgttccagtg | CTCATTTTGGAGG | ssRNA | |
| | | | GGAGGGTAATCACAAC | tggagttc | GTAATCACAAC | | |
| | | | (SEQ ID. NO: 1263) | (SEQ ID. | (SEQ ID. | | |
| | | | | NO: | NO: 1265) | | |
| | | | | 1264) | | | |
| 1b | zika_8 | CcaCas13b | agtgcttctttgttgttccagtgtg | agtgcttcttt | GTTGGAACTGCT | Zika | 1b |
| | | | gagttGTTGGAACTGCTCTCATTTT | gttgttccagt | CTCATTTTGGAGG | ssRNA | |
| | | | GGAGGGTAATCACAAC | gtggagtt | GTAATCACAAC | | |
| | | | (SEQ ID. NO: 1266) | (SEQ ID. | (SEQ ID. | | |
| | | | | NO: | NO: 1268) | | |
| | | | | 1267) | | | |
| 1b | zika_9 | CcaCas13b | cagtgcttctttgttgttccagtgt | cagtgcttctt | GTTGGAACTGCT | Zika | 1b |
| | | | ggagtGTTGGAACTGCTCTCATTTT | tgttgttccag | CTCATTTTGGAGG | ssRNA | |
| | | | GGAGGGTAATCACAAC | tgtggagt | GTAATCACAAC | | |
| | | | (SEQ ID. NO: 1269) | (SEQ ID. | (SEQ ID. | | |
| | | | | NO: | NO: 1271) | | |
| | | | | 1270) | | | |
| 1b | zika_10 | CcaCas13b | ccagtgcttctttgttgttccagtg | ccagtgcttc | GTTGGAACTGCT | Zika | 1b |
| | | | tggagGTTGGAACTGCTCTCATTTT | tttgttgttcc | CTCATTTTGGAGG | ssRNA | |
| | | | GGAGGGTAATCACAAC | agtgtggag | GTAATCACAAC | | |
| | | | (SEQ ID. NO: 1272) | (SEQ ID. | (SEQ ID. | | |
| | | | | NO: | NO: 1274) | | |
| | | | | 1273) | | | |
| 1b | zika_11 | CcaCas13b | accagtgcttctttgttgttccagt | accagtgctt | GTTGGAACTGCT | Zika | 1b |
| | | | gtggaGTTGGAACTGCTCTCATTTT | ctttgttgttc | CTCATTTTGGAGG | ssRNA | |
| | | | GGAGGGTAATCACAAC | cagtgtgga | GTAATCACAAC | | |
| | | | (SEQ ID. NO: 1275) | (SEQ ID. | (SEQ ID. | | |
| | | | | NO: | NO: 1277) | | |
| | | | | 1276) | | | |
| 1b | zika_12 | CcaCas13b | taccagtgcttctttgttgttccag | taccagtgct | GTTGGAACTGCT | Zika | 1b |
| | | | tgtggGTTGGAACTGCTCTCATTTT | tctttgttgtt | CTCATTTTGGAGG | ssRNA | |
| | | | GGAGGGTAATCACAAC | ccagtgtgg | GTAATCACAAC | | |
| | | | (SEQ ID. NO: 1278) | (SEQ ID. | (SEQ ID. | | |
| | | | | NO: | NO: 1280) | | |
| | | | | 1279) | | | |
| 1b | zika_13 | CcaCas13b | ctaccagtgcttctttgttgttcca | ctaccagtgc | GTTGGAACTGCT | Zika | 1b |
| | | | gtgtgGTTGGAACTGCTCTCATTTT | ttctttgttgt | CTCATTTTGGAGG | ssRNA | |
| | | | GGAGGGTAATCACAAC | tccagtgtg | GTAATCACAAC | | |
| | | | (SEQ ID. NO: 1281) | (SEQ ID. | (SEQ ID. | | |
| | | | | NO: | NO: 1283) | | |
| | | | | 1282) | | | |
| 1b | zika_14 | CcaCas13b | tctaccagtgcttctttgttgttcc | tctaccagtg | GTTGGAACTGCT | Zika | 1b |
| | | | agtgtGTTGGAACTGCTCTCATTTT | cttctttgttg | CTCATTTTGGAGG | ssRNA | |
| | | | GGAGGGTAATCACAAC | ttccagtgt | GTAATCACAAC | | |
| | | | (SEQ ID. NO: 1284) | (SEQ ID. | (SEQ ID. | | |
| | | | | NO: | NO: 1286) | | |
| | | | | 1285) | | | |
| 1b | zika_15 | CcaCas13b | ctctaccagtgcttctttgttgttc | ctctaccagt | GTTGGAACTGCT | Zika | 1b |
| | | | cagtgGTTGGAACTGCTCTCATTTT | gcttctttgtt | CTCATTTTGGAGG | ssRNA | |
| | | | GGAGGGTAATCACAAC | gttccagtg | GTAATCACAAC | | |
| | | | (SEQ ID. NO: 1287) | (SEQ ID. | (SEQ ID. | | |
| | | | | NO: | NO: 1289) | | |
| | | | | 1288) | | | |
| 1b | zika_16 | CcaCas13b | actctaccagtgcttctttgttgtt | actctaccag | GTTGGAACTGCT | Zika | 1b |
| | | | ccagtGTTGGAACTGCTCTCATTTT | tgcttctttgt | CTCATTTTGGAGG | ssRNA | |
| | | | GGAGGGTAATCACAAC | tgttccagt | GTAATCACAAC | | |
| | | | (SEQ ID. NO: 1290) | (SEQ ID. | (SEQ ID. | | |
| | | | | NO: | NO: 1292) | | |
| | | | | 1291) | | | |
| 1b | zika_17 | CcaCas13b | aactctaccagtgcttctttgttgt | aactctacca | GTTGGAACTGCT | Zika | 1b |
| | | | tccagGTTGGAACTGCTCTCATTTT | gtgcttctttg | CTCATTTTGGAGG | ssRNA | |
| | | | GGAGGGTAATCACAAC | ttgttccag | GTAATCACAAC | | |
| | | | (SEQ ID. NO: 1293) | (SEQ ID. | (SEQ ID. | | |
| | | | | NO: | NO: 1295) | | |
| | | | | 1294) | | | |
| 1b | zika_18 | CcaCas13b | gaactctaccagtgcttctttgttg | gaactctacc | GTTGGAACTGCT | Zika | 1b |
| | | | ttccaGTTGGAACTGCTCTCATTTT | agtgcttcttt | CTCATTTTGGAGG | ssRNA | |
| | | | GGAGGGTAATCACAAC | gttgttcca | GTAATCACAAC | | |
| | | | (SEQ ID. NO: 1296) | (SEQ ID. | (SEQ ID. | | |
| | | | | NO: | NO: 1298) | | |
| | | | | 1297) | | | |
| 1b | zika_19 | CcaCas13b | tgaactctaccagtgcttctttgtt | tgaactctac | GTTGGAACTGCT | Zika | 1b |
| | | | gttccGTTGGAACTGCTCTCATTTT | cagtgcttctt | CTCATTTTGGAGG | ssRNA | |
| | | | GGAGGGTAATCACAAC | tgttgttcc | GTAATCACAAC | | |
| | | | (SEQ ID. NO: 1299) | (SEQ ID. | (SEQ ID. | | |
| | | | | NO: | NO: 1301) | | |
| | | | | 1300) | | | |
| 1b | zika_20 | CcaCas13b | cttgaactctaccagtgcttctttg | cttgaactct | GTTGGAACTGCT | Zika | 1b |
| | | | ttgttGTTGGAACTGCTCTCATTTT | accagtgctt | CTCATTTTGGAGG | ssRNA | |
| | | | GGAGGGTAATCACAAC | ctttgttgtt | GTAATCACAAC | | |
| | | | (SEQ ID. NO: 1302) | (SEQ ID. | (SEQ ID. | | |
| | | | | NO: | NO: 1304) | | |
| | | | | 1303) | | | |
| 1b | zika_21 | CcaCas13b | tccttgaactctaccagtgcttctt | tccttgaact | GTTGGAACTGCT | Zika | 1b |
| | | | tgttgGTTGGAACTGCTCTCATTTT | ctaccagtgc | CTCATTTTGGAGG | ssRNA | |
| | | | GGAGGGTAATCACAAC | ttetttgttg | GTAATCACAAC | | |
| | | | (SEQ ID. NO: 1305) | (SEQ ID. | (SEQ ID. | | |
| | | | | NO: | NO: 1307) | | |
| | | | | 1306) | | | |
| 1b | zika_22 | CcaCas13b | cgtccttgaactctaccagtgcttc | cgtccttgaa | GTTGGAACTGCT | Zika | 1b |
| | | | tttgtGTTGGAACTGCTCTCATTTT | ctctaccagt | CTCATTTTGGAGG | ssRNA | |
| | | | GGAGGGTAATCACAAC | gcttctttgt | GTAATCACAAC | | |
| | | | (SEQ ID. NO: 1308) | (SEQ ID. | (SEQ ID. | | |
| | | | | NO: | NO: 1310) | | |
| | | | | 1309) | | | |
| 1b | zika_23 | CcaCas13b | tgcgtccttgaactctaccagtgct | tgcgtccttg | GTTGGAACTGCT | Zika | 1b |
| | | | tctttGTTGGAACTGCTCTCATTTT | aactctacca | CTCATTTTGGAGG | ssRNA | |
| | | | GGAGGGTAATCACAAC | gtgcttcttt | GTAATCACAAC | | |
| | | | (SEQ ID. NO: 1311) | (SEQ ID. | (SEQ ID. | | |
| | | | | NO: | NO: 1313) | | |
| | | | | 1312) | | | |
| 1b | zika_24 | CcaCas13b | tgtgcgtccttgaactctaccagtg | tgtgcgtcctt | GTTGGAACTGCT | Zika | 1b |
| | | | cttctGTTGGAACTGCTCTCATTTT | gaactctacc | CTCATTTTGGAGG | ssRNA | |
| | | | GGAGGGTAATCACAAC | agtgcttct | GTAATCACAAC | | |
| | | | (SEQ ID. NO: 1314) | (SEQ ID. | (SEQ ID. | | |
| | | | | NO: | NO: 1316) | | |
| | | | | 1315) | | | |
| 1b | zika_25 | CcaCas13b | catgtgcgtccttgaactctaccag | catgtgcgtc | GTTGGAACTGCT | Zika | 1b |
| | | | tgcttGTTGGAACTGCTCTCATTTT | cttgaactct | CTCATTTTGGAGG | ssRNA | |
| | | | GGAGGGTAATCACAAC | accagtgctt | GTAATCACAAC | | |
| | | | (SEQ ID. NO: 1317) | (SEQ ID. | (SEQ ID. | | |
| | | | | NO: | NO: 1319) | | |
| | | | | 1318) | | | |
| 1b | zika_26 | CcaCas13b | ggcatgtgcgtccttgaactctacc | ggcatgtgc | GTTGGAACTGCT | Zika | 1b |
| | | | agtgcGTTGGAACTGCTCTCATTTT | gtccttgaac | CTCATTTTGGAGG | ssRNA | |
| | | | GGAGGGTAATCACAAC | tctaccagtg | GTAATCACAAC | | |
| | | | (SEQ ID. NO: 1320) | c (SEQ | (SEQ ID. | | |
| | | | | ID. NO: | NO: 1322) | | |
| | | | | 1321) | | | |
| 1b | zika_27 | CcaCas13b | ttggcatgtgcgtccttgaactcta | ttggcatgtg | GTTGGAACTGCT | Zika | 1b |
| | | | ccagtGTTGGAACTGCTCTCATTTT | cgtccttgaa | CTCATTTTGGAGG | ssRNA | |
| | | | GGAGGGTAATCACAAC | ctctaccagt | GTAATCACAAC | | |
| | | | (SEQ ID. NO: 1323) | (SEQ ID. | (SEQ ID. | | |
| | | | | NO: | NO: 1325) | | |
| | | | | 1324) | | | |
| 1b | zika_28 | CcaCas13b | ttttggcatgtgcgtccttgaactc | ttttggcatgt | GTTGGAACTGCT | Zika | 1b |
| | | | taccaGTTGGAACTGCTCTCATTTT | gcgtccttga | CTCATTTTGGAGG | ssRNA | |
| | | | GGAGGGTAATCACAAC | actctacca | GTAATCACAAC | | |
| | | | (SEQ ID. NO: 1326) | (SEQ ID. | (SEQ ID. | | |
| | | | | NO: | NO: 1328) | | |
| | | | | 1327) | | | |
| 1b | zika_29 | CcaCas13b | ccttttggcatgtgcgtccttgaac | ccttttggcat | GTTGGAACTGCT | Zika | 1b |
| | | | tctacGTTGGAACTGCTCTCATTTT | gtgcgtcctt | CTCATTTTGGAGG | ssRNA | |
| | | | GGAGGGTAATCACAAC | gaactctac | GTAATCACAAC | | |
| | | | (SEQ ID. NO: 1329) | (SEQ ID. | (SEQ ID. | | |
| | | | | NO: | NO: 1331) | | |
| | | | | 1330) | | | |
| 1b | zika_30 | CcaCas13b | tgccttttggcatgtgcgtccttga | tgccttttggc | GTTGGAACTGCT | Zika | 1b |
| | | | actctGTTGGAACTGCTCTCATTTT | atgtgcgtcc | CTCATTTTGGAGG | ssRNA | |
| | | | GGAGGGTAATCACAAC | ttgaactct | GTAATCACAAC | | |
| | | | (SEQ ID. NO: 1332) | (SEQ ID. | (SEQ ID. | | |
| | | | | NO: | NO: 1334) | | |
| | | | | 1333) | | | |
| 1b | zika_31 | CcaCas13b | tttgccttttggcatgtgcgtcctt | tttgccttttg | GTTGGAACTGCT | Zika | 1b |
| | | | gaactGTTGGAACTGCTCTCATTTT | gcatgtgcgt | CTCATTTTGGAGG | ssRNA | |
| | | | GGAGGGTAATCACAAC | ccttgaact | GTAATCACAAC | | |
| | | | (SEQ ID. NO: 1335) | (SEQ ID. | (SEQ ID. | | |
| | | | | NO: | NO: 1337) | | |
| | | | | 1336) | | | |
| 1b | zika_32 | CcaCas13b | agtttgccttttggcatgtgcgtcc | agtttgccttt | GTTGGAACTGCT | Zika | 1b |
| | | | ttgaaGTTGGAACTGCTCTCATTTT | tggcatgtgc | CTCATTTTGGAGG | ssRNA | |
| | | | GGAGGGTAATCACAAC | gtccttgaa | GTAATCACAAC | | |
| | | | (SEQ ID. NO: 1338) | (SEQ ID. | (SEQ ID. | | |
| | | | | NO: | NO: 1340) | | |
| | | | | 1339) | | | |
| 1b | zika_33 | CcaCas13b | acagtttgccttttggcatgtgcgt | acagtttgcc | GTTGGAACTGCT | Zika | 1b |
| | | | ccttgGTTGGAACTGCTCTCATTTT | ttttggcatgt | CTCATTTTGGAGG | ssRNA | |
| | | | GGAGGGTAATCACAAC | gcgtccttg | GTAATCACAAC | | |
| | | | (SEQ ID. NO: 1341) | (SEQ ID. | (SEQ ID. | | |
| | | | | NO: | NO: 1343) | | |
| | | | | 1342) | | | |
| 1b | zika_34 | CcaCas13b | cgacagtttgccttttggcatgtgc | cgacagtttg | GTTGGAACTGCT | Zika | 1b |
| | | | gtcctGTTGGAACTGCTCTCATTTT | ccttttggcat | CTCATTTTGGAGG | ssRNA | |
| | | | GGAGGGTAATCACAAC | gtgcgtcct | GTAATCACAAC | | |
| | | | (SEQ ID. NO: 1344) | (SEQ ID. | (SEQ ID. | | |
| | | | | NO: | NO: 1346) | | |
| | | | | 1345) | | | |
| 1b | zika_35 | CcaCas13b | cacgacagtttgccttttggcatgt | cacgacagtt | GTTGGAACTGCT | Zika | 1b |
| | | | gcgtcGTTGGAACTGCTCTCATTTT | tgccttttggc | CTCATTTTGGAGG | ssRNA | |
| | | | GGAGGGTAATCACAAC | atgtgcgtc | GTAATCACAAC | | |
| | | | (SEQ ID. NO: 1347) | (SEQ ID. | (SEQ ID. | | |
| | | | | NO: | NO: 1349) | | |
| | | | | 1348) | | | |
| 1b | zika_36 | CcaCas13b | accacgacagtttgccttttggcat | accacgaca | GTTGGAACTGCT | Zika | 1b |
| | | | gtgcgGTTGGAACTGCTCTCATTTT | gtttgcctttt | CTCATTTTGGAGG | ssRNA | |
| | | | GGAGGGTAATCACAAC | ggcatgtgc | GTAATCACAAC | | |
| | | | (SEQ ID. NO: 1350) | g (SEQ | (SEQ ID. | | |
| | | | | ID. NO: | NO: 1352) | | |
| | | | | 1351) | | | |
| 1b | zika_37 | CcaCas13b | gaaccacgacagtttgccttttggc | gaaccacga | GTTGGAACTGCT | Zika | 1b |
| | | | atgtgGTTGGAACTGCTCTCATTTT | cagtttgcctt | CTCATTTTGGAGG | ssRNA | |
| | | | GGAGGGTAATCACAAC | ttggcatgtg | GTAATCACAAC | | |
| | | | (SEQ ID. NO: 1353) | (SEQ ID. | (SEQ ID. | | |
| | | | | NO: | NO: 1355) | | |
| | | | | 1354) | | | |
| 1b | zika_38 | CcaCas13b | tagaaccacgacagtttgccttttg | tagaaccac | GTTGGAACTGCT | Zika | 1b |
| | | | gcatgGTTGGAACTGCTCTCATTTT | gacagtttgc | CTCATTTTGGAGG | ssRNA | |
| | | | GGAGGGTAATCACAAC | cttttggcatg | GTAATCACAAC | | |
| | | | (SEQ ID. NO: 1356) | (SEQ ID. | (SEQ ID. | | |
| | | | | NO: | NO: 1358) | | |
| | | | | 1357) | | | |
| 1b | zika_39 | CcaCas13b | cctagaaccacgacagtttgccttt | cctagaacc | GTTGGAACTGCT | Zika | 1b |
| | | | tggcaGTTGGAACTGCTCTCATTTT | acgacagttt | CTCATTTTGGAGG | ssRNA | |
| | | | GGAGGGTAATCACAAC | gccttttggc | GTAATCACAAC | | |
| | | | (SEQ ID. NO: 1359) | a (SEQ | (SEQ ID. | | |
| | | | | ID. NO: | NO: 1361) | | |
| | | | | 1360) | | | |
| 1b | zika_40 | CcaCas13b | tccctagaaccacgacagtttgcct | tccctagaac | GTTGGAACTGCT | Zika | 1b |
| | | | tttggGTTGGAACTGCTCTCATTTT | cacgacagtt | CTCATTTTGGAGG | ssRNA | |
| | | | GGAGGGTAATCACAAC | tgccttttgg | GTAATCACAAC | | |
| | | | (SEQ ID. NO: 1362) | (SEQ ID. | (SEQ ID. | | |
| | | | | NO: | NO: 1364) | | |
| | | | | 1363) | | | |
| 1b | zika_41 | CcaCas13b | actccctagaaccacgacagtttgc | actccctaga | GTTGGAACTGCT | Zika | 1b |
| | | | cttttGTTGGAACTGCTCTCATTTT | accacgaca | CTCATTTTGGAGG | ssRNA | |
| | | | GGAGGGTAATCACAAC | gtttgcctttt | GTAATCACAAC | | |
| | | | (SEQ ID. NO: 1365) | (SEQ ID. | (SEQ ID. | | |
| | | | | NO: | NO: 1367) | | |
| | | | | 1366) | | | |
| 1b | zika_42 | CcaCas13b | tgactccctagaaccacgacagttt | tgactcccta | GTTGGAACTGCT | Zika | 1b |
| | | | gccttGTTGGAACTGCTCTCATTTT | gaaccacga | CTCATTTTGGAGG | ssRNA | |
| | | | GGAGGGTAATCACAAC | cagtttgcctt | GTAATCACAAC | | |
| | | | (SEQ ID. NO: 1368) | (SEQ ID. | (SEQ ID. | | |
| | | | | NO: | NO: 1370) | | |
| | | | | 1369) | | | |
| 1b | zika_43 | CcaCas13b | cttgactccctagaaccacgacagt | cttgactccc | GTTGGAACTGCT | Zika | 1b |
| | | | ttgccGTTGGAACTGCTCTCATTTT | tagaaccac | CTCATTTTGGAGG | ssRNA | |
| | | | GGAGGGTAATCACAAC | gacagtttgc | GTAATCACAAC | | |
| | | | (SEQ ID. NO: 1371) | c (SEQ | (SEQ ID. | | |
| | | | | ID. NO: | NO: 1373) | | |
| | | | | 1372) | | | |
| 1b | zika_44 | CcaCas13b | ttcttgactccctagaaccacgaca | ttcttgactcc | GTTGGAACTGCT | Zika | 1b |
| | | | gtttgGTTGGAACTGCTCTCATTTT | ctagaacca | CTCATTTTGGAGG | ssRNA | |
| | | | GGAGGGTAATCACAAC | cgacagtttg | GTAATCACAAC | | |
| | | | (SEQ ID. NO: 1374) | (SEQ ID. | (SEQ ID. | | |
| | | | | NO: | NO: 1376) | | |
| | | | | 1375) | | | |
| 1b | zika_45 | CcaCas13b | ccttcttgactccctagaaccacga | ccttcttgact | GTTGGAACTGCT | Zika | 1b |
| | | | cagttGTTGGAACTGCTCTCATTTT | ccctagaac | CTCATTTTGGAGG | ssRNA | |
| | | | GGAGGGTAATCACAAC | cacgacagtt | GTAATCACAAC | | |
| | | | (SEQ ID. NO: 1377) | (SEQ ID. | (SEQ ID. | | |
| | | | | NO: | NO: 1379) | | |
| | | | | 1378) | | | |
| 1b | zika_46 | CcaCas13b | ctccttcttgactccctagaaccac | ctccttcttga | GTTGGAACTGCT | Zika | 1b |
| | | | gacagGTTGGAACTGCTCTCATTTT | ctccctagaa | CTCATTTTGGAGG | ssRNA | |
| | | | GGAGGGTAATCACAAC | ccacgacag | GTAATCACAAC | | |
| | | | (SEQ ID. NO: 1380) | (SEQ ID. | (SEQ ID. | | |
| | | | | NO: | NO: 1382) | | |
| | | | | 1381) | | | |
| 1b | zika_47 | CcaCas13b | tgctccttcttgactccctagaacc | tgctccttctt | GTTGGAACTGCT | Zika | 1b |
| | | | acgacGTTGGAACTGCTCTCATTTT | gactccctag | CTCATTTTGGAGG | ssRNA | |
| | | | GGAGGGTAATCACAAC | aaccacgac | GTAATCACAAC | | |
| | | | (SEQ ID. NO: 1383) | (SEQ ID. | (SEQ ID. | | |
| | | | | NO: | NO: 1385) | | |
| | | | | 1384) | | | |
| 1b | zika_48 | CcaCas13b | actgctccttcttgactccctagaa | actgctcctt | GTTGGAACTGCT | Zika | 1b |
| | | | ccacgGTTGGAACTGCTCTCATTTT | cttgactccc | CTCATTTTGGAGG | ssRNA | |
| | | | GGAGGGTAATCACAAC | tagaaccac | GTAATCACAAC | | |
| | | | (SEQ ID. NO: 1386) | g (SEQ | (SEQ ID. | | |
| | | | | ID. NO: | NO: 1388) | | |
| | | | | 1387) | | | |
| 1b | zika_49 | CcaCas13b | gaactgctccttcttgactccctag | gaactgctcc | GTTGGAACTGCT | Zika | 1b |
| | | | aaccaGTTGGAACTGCTCTCATTTT | ttcttgactcc | CTCATTTTGGAGG | ssRNA | |
| | | | GGAGGGTAATCACAAC | ctagaacca | GTAATCACAAC | | |
| | | | (SEQ ID. NO: 1389) | (SEQ ID. | (SEQ ID. | | |
| | | | | NO: | NO: 1391) | | |
| | | | | 1390) | | | |
| 1b | zika_50 | CcaCas13b | gtgaactgctccttcttgactccct | gtgaactgct | GTTGGAACTGCT | Zika | 1b |
| | | | agaacGTTGGAACTGCTCTCATTTT | ccttcttgact | CTCATTTTGGAGG | ssRNA | |
| | | | GGAGGGTAATCACAAC | ccctagaac | GTAATCACAAC | | |
| | | | (SEQ ID. NO: 1392) | (SEQ ID. | (SEQ ID. | | |
| | | | | NO: | NO: 1394) | | |
| | | | | 1393) | | | |
| 1b | zika_51 | CcaCas13b | gtgtgaactgctccttcttgactcc | gtgtgaactg | GTTGGAACTGCT | Zika | 1b |
| | | | ctagaGTTGGAACTGCTCTCATTTT | ctccttcttga | CTCATTTTGGAGG | ssRNA | |
| | | | GGAGGGTAATCACAAC | ctccctaga | GTAATCACAAC | | |
| | | | (SEQ ID. NO: 1395) | (SEQ ID. | (SEQ ID. | | |
| | | | | NO: | NO: 1397) | | |
| | | | | 1396) | | | |
| 1b | zika_52 | CcaCas13b | ccgtgtgaactgctccttcttgact | ccgtgtgaa | GTTGGAACTGCT | Zika | 1b |
| | | | ccctaGTTGGAACTGCTCTCATTTT | ctgctccttct | CTCATTTTGGAGG | ssRNA | |
| | | | GGAGGGTAATCACAAC | tgactcccta | GTAATCACAAC | | |
| | | | (SEQ ID. NO: 1398) | (SEQ ID. | (SEQ ID. | | |
| | | | | NO: | NO: 1400) | | |
| | | | | 1399) | | | |
| 1b | zika_53 | CcaCas13b | ggccgtgtgaactgctccttcttga | ggccgtgtg | GTTGGAACTGCT | Zika | 1b |
| | | | ctcccGTTGGAACTGCTCTCATTTT | aactgctcct | CTCATTTTGGAGG | ssRNA | |
| | | | GGAGGGTAATCACAAC | tcttgactcc | GTAATCACAAC | | |
| | | | (SEQ ID. NO: 1401) | c (SEQ | (SEQ ID. | | |
| | | | | ID. NO: | NO: 1403) | | |
| | | | | 1402) | | | |
| 1b | zika_54 | CcaCas13b | agggccgtgtgaactgctccttctt | agggccgtg | GTTGGAACTGCT | Zika | 1b |
| | | | gactcGTTGGAACTGCTCTCATTTT | tgaactgctc | CTCATTTTGGAGG | ssRNA | |
| | | | GGAGGGTAATCACAAC | cttcttgactc | GTAATCACAAC | | |
| | | | (SEQ ID. NO: 1404) | (SEQ ID. | (SEQ ID. | | |
| | | | | NO: | NO: 1406) | | |
| | | | | 1405) | | | |
| 1b | zika_55 | CcaCas13b | caagggccgtgtgaactgctccttc | caagggccg | GTTGGAACTGCT | Zika | 1b |
| | | | ttgacGTTGGAACTGCTCTCATTTT | tgtgaactgc | CTCATTTTGGAGG | ssRNA | |
| | | | GGAGGGTAATCACAAC | tccttcttgac | GTAATCACAAC | | |
| | | | (SEQ ID. NO: 1407) | (SEQ ID. | (SEQ ID. | | |
| | | | | NO: | NO: 1409) | | |
| | | | | 1408) | | | |
| 1b | zika_56 | CcaCas13b | agcaagggccgtgtgaactgctcct | agcaagggc | GTTGGAACTGCT | Zika | 1b |
| | | | tcttgGTTGGAACTGCTCTCATTTT | cgtgtgaact | CTCATTTTGGAGG | ssRNA | |
| | | | GGAGGGTAATCACAAC | gctccttcttg | GTAATCACAAC | | |
| | | | (SEQ ID. NO: 1410) | (SEQ ID. | (SEQ ID. | | |
| | | | | NO: | NO: 1412) | | |
| | | | | 1411) | | | |
| 1b | zika_57 | CcaCas13b | ccagcaagggccgtgtgaactgctc | ccagcaagg | GTTGGAACTGCT | Zika | 1b |
| | | | cttctGTTGGAACTGCTCTCATTTT | gccgtgtga | CTCATTTTGGAGG | ssRNA | |
| | | | GGAGGGTAATCACAAC | actgctcctt | GTAATCACAAC | | |
| | | | (SEQ ID. NO: 1413) | ct (SEQ | (SEQ ID. | | |
| | | | | ID. NO: | NO: 1415) | | |
| | | | | 1414) | | | |
| 1b | zika_58 | CcaCas13b | ctccagcaagggccgtgtgaactgc | ctccagcaa | GTTGGAACTGCT | Zika | 1b |
| | | | tccttGTTGGAACTGCTCTCATTTT | gggccgtgt | CTCATTTTGGAGG | ssRNA | |
| | | | GGAGGGTAATCACAAC | gaactgctcc | GTAATCACAAC | | |
| | | | (SEQ ID. NO: 1416) | tt (SEQ | (SEQ ID. | | |
| | | | | ID. NO: | NO: 1418) | | |
| | | | | 1417) | | | |
| 1b | zika_59 | CcaCas13b | agctccagcaagggccgtgtgaact | agctccagc | GTTGGAACTGCT | Zika | 1b |
| | | | gctccGTTGGAACTGCTCTCATTTT | aagggccgt | CTCATTTTGGAGG | ssRNA | |
| | | | GGAGGGTAATCACAAC | gtgaactgct | GTAATCACAAC | | |
| | | | (SEQ ID. NO: 1419) | cc (SEQ | (SEQ ID. | | |
| | | | | ID. NO: | NO: 1421) | | |
| | | | | 1420) | | | |
| 1b | zika_60 | CcaCas13b | agagctccagcaagggccgtgtgaa | agagctcca | GTTGGAACTGCT | Zika | 1b |
| | | | ctgctGTTGGAACTGCTCTCATTTT | gcaagggcc | CTCATTTTGGAGG | ssRNA | |
| | | | GGAGGGTAATCACAAC | gtgtgaactg | GTAATCACAAC | | |
| | | | (SEQ ID. NO: 1422) | ct (SEQ | (SEQ ID. | | |
| | | | | ID. NO: | NO: 1424) | | |
| | | | | 1423) | | | |
| 1b | zika_61 | CcaCas13b | ccagagctccagcaagggccgtgtg | ccagagctc | GTTGGAACTGCT | Zika | 1b |
| | | | aactgGTTGGAACTGCTCTCATTTT | cagcaaggg | CTCATTTTGGAGG | ssRNA | |
| | | | GGAGGGTAATCACAAC | ccgtgtgaa | GTAATCACAAC | | |
| | | | (SEQ ID. NO: 1425) | ctg (SEQ | (SEQ ID. | | |
| | | | | ID. NO: | NO: 1427) | | |
| | | | | 1426) | | | |
| 1b | zika_62 | CcaCas13b | ctccagagctccagcaagggccgtg | ctccagagct | GTTGGAACTGCT | Zika | 1b |
| | | | tgaacGTTGGAACTGCTCTCATTTT | ccagcaagg | CTCATTTTGGAGG | ssRNA | |
| | | | GGAGGGTAATCACAAC | gccgtgtga | GTAATCACAAC | | |
| | | | (SEQ ID. NO: 1428) | ac (SEQ | (SEQ ID. | | |
| | | | | ID. NO: | NO: 1430) | | |
| | | | | 1429) | | | |
| 1b | zika_63 | CcaCas13b | gcctccagagctccagcaagggccg | gcctccaga | GTTGGAACTGCT | Zika | lb |
| | | | tgtgaGTTGGAACTGCTCTCATTTT | gctccagca | CTCATTTTGGAGG | ssRNA | |
| | | | GGAGGGTAATCACAAC | agggccgtg | GTAATCACAAC | | |
| | | | (SEQ ID. NO: 1431) | tga (SEQ | (SEQ ID. | | |
| | | | | ID. NO: | NO: 1433) | | |
| | | | | 1432) | | | |
| 1b | zika_64 | CcaCas13b | cagcctccagagctccagcaagggc | cagcctcca | GTTGGAACTGCT | Zika | 1b |
| | | | cgtgtGTTGGAACTGCTCTCATTTT | gagctccag | CTCATTTTGGAGG | ssRNA | |
| | | | GGAGGGTAATCACAAC | caagggccg | GTAATCACAAC | | |
| | | | (SEQ ID. NO: 1434) | tgt (SEQ | (SEQ ID. | | |
| | | | | ID. NO: | NO: 1436) | | |
| | | | | 1435) | | | |
| 1b | zika_65 | CcaCas13b | ctcagcctccagagctccagcaagg | ctcagcctcc | GTTGGAACTGCT | Zika | 1b |
| | | | gccgtGTTGGAACTGCTCTCATTTT | agagctcca | CTCATTTTGGAGG | ssRNA | |
| | | | GGAGGGTAATCACAAC | gcaagggcc | GTAATCACAAC | | |
| | | | (SEQ ID. NO: 1437) | gt (SEQ | (SEQ ID. | | |
| | | | | ID. NO: | NO: 1439) | | |
| | | | | 1438) | | | |
| 1b | zika_66 | CcaCas13b | atctcagcctccagagctccagcaa | atctcagcct | GTTGGAACTGCT | Zika | 1b |
| | | | gggccGTTGGAACTGCTCTCATTTT | ccagagctc | CTCATTTTGGAGG | ssRNA | |
| | | | GGAGGGTAATCACAAC | cagcaaggg | GTAATCACAAC | | |
| | | | (SEQ ID. NO: 1440) | cc (SEQ | (SEQ ID. | | |
| | | | | ID. NO: | NO: 1442) | | |
| | | | | 1441) | | | |
| 1b | zika_67 | CcaCas13b | ccatctcagcctccagagctccagc | ccatctcagc | GTTGGAACTGCT | Zika | 1b |
| | | | aagggGTTGGAACTGCTCTCATTTT | ctccagagct | CTCATTTTGGAGG | ssRNA | |
| | | | GGAGGGTAATCACAAC | ccagcaagg | GTAATCACAAC | | |
| | | | (SEQ ID. NO: 1443) | g (SEQ | (SEQ ID. | | |
| | | | | ID. NO: | NO: 1445) | | |
| | | | | 1444) | | | |
| 1b | zika_68 | CcaCas13b | tccatctcagcctccagagctccag | tccatctcag | GTTGGAACTGCT | Zika | 1b |
| | | | caaggGTTGGAACTGCTCTCATTTT | cctccagag | CTCATTTTGGAGG | ssRNA | |
| | | | GGAGGGTAATCACAAC | ctccagcaa | GTAATCACAAC | | |
| | | | (SEQ ID. NO: 1446) | gg (SEQ | (SEQ ID. | | |
| | | | | ID. NO: | NO: 1448) | | |
| | | | | 1447) | | | |
| 1b | zika_69 | CcaCas13b | atccatctcagcctccagagctcca | atccatctca | GTTGGAACTGCT | Zika | 1b |
| | | | gcaagGTTGGAACTGCTCTCATTTT | gcctccaga | CTCATTTTGGAGG | ssRNA | |
| | | | GGAGGGTAATCACAAC | gctccagca | GTAATCACAAC | | |
| | | | (SEQ ID. NO: 1449) | ag (SEQ | (SEQ ID. | | |
| | | | | ID. NO: | NO: 1451) | | |
| | | | | 1450) | | | |
| 1b | zika_70 | CcaCas13b | catccatctcagcctccagagctcc | catccatctc | GTTGGAACTGCT | Zika | 1b |
| | | | agcaaGTTGGAACTGCTCTCATTTT | agcctccag | CTCATTTTGGAGG | ssRNA | |
| | | | GGAGGGTAATCACAAC | agctccagc | GTAATCACAAC | | |
| | | | (SEQ ID. NO: 1452) | aa (SEQ | (SEQ ID. | | |
| | | | | ID. NO: | NO: 1454) | | |
| | | | | 1453) | | | |
| 1b | zika_71 | CcaCas13b | ccatccatctcagcctccagagctc | ccatccatct | GTTGGAACTGCT | Zika | 1b |
| | | | cagcaGTTGGAACTGCTCTCATTTT | cagcctcca | CTCATTTTGGAGG | ssRNA | |
| | | | GGAGGGTAATCACAAC | gagctccag | GTAATCACAAC | | |
| | | | (SEQ ID. NO: 1455) | ca (SEQ | (SEQ ID. | | |
| | | | | ID. NO: | NO: 1457) | | |
| | | | | 1456) | | | |
| 1b | zika_72 | CcaCas13b | accatccatctcagcctccagagct | accatccatc | GTTGGAACTGCT | Zika | 1b |
| | | | ccagcGTTGGAACTGCTCTCATTTT | tcagcctcca | CTCATTTTGGAGG | ssRNA | |
| | | | GGAGGGTAATCACAAC | gagctccag | GTAATCACAAC | | |
| | | | (SEQ ID. NO: 1458) | c (SEQ | (SEQ ID. | | |
| | | | | ID. NO: | NO: 1460) | | |
| | | | | 1459) | | | |
| 1b | zika_73 | CcaCas13b | caccatccatctcagcctccagagc | caccatccat | GTTGGAACTGCT | Zika | 1b |
| | | | tccagGTTGGAACTGCTCTCATTTT | ctcagcctcc | CTCATTTTGGAGG | ssRNA | |
| | | | GGAGGGTAATCACAAC | agagctcca | GTAATCACAAC | | |
| | | | (SEQ ID. NO: 1461) | g (SEQ | (SEQ ID. | | |
| | | | | ID. NO: | NO: 1463) | | |
| | | | | 1462) | | | |
| 1b | zika_74 | CcaCas13b | gcaccatccatctcagcctccagag | gcaccatcc | GTTGGAACTGCT | Zika | 1b |
| | | | ctccaGTTGGAACTGCTCTCATTTT | atctcagcct | CTCATTTTGGAGG | ssRNA | |
| | | | GGAGGGTAATCACAAC | ccagagctc | GTAATCACAAC | | |
| | | | (SEQ ID. NO: 1464) | ca (SEQ | (SEQ ID. | | |
| | | | | ID. NO: | NO: 1466) | | |
| | | | | 1465) | | | |
| 1b | zika_75 | CcaCas13b | tgcaccatccatctcagcctccaga | tgcaccatcc | GTTGGAACTGCT | Zika | 1b |
| | | | gctccGTTGGAACTGCTCTCATTTT | atctcagcct | CTCATTTTGGAGG | ssRNA | |
| | | | GGAGGGTAATCACAAC | ccagagctc | GTAATCACAAC | | |
| | | | (SEQ ID. NO: 1467) | c (SEQ | (SEQ ID. | | |
| | | | | ID. NO: | NO: 1469) | | |
| | | | | 1468) | | | |
| 1b | zika_76 | CcaCas13b | ttgcaccatccatctcagcctccag | ttgcaccatc | GTTGGAACTGCT | Zika | 1b |
| | | | agctcGTTGGAACTGCTCTCATTTT | catctcagcc | CTCATTTTGGAGG | ssRNA | |
| | | | GGAGGGTAATCACAAC | tccagagctc | GTAATCACAAC | | |
| | | | (SEQ ID. NO: 1470) | (SEQ ID. | (SEQ ID. | | |
| | | | | NO: | NO: 1472) | | |
| | | | | 1471) | | | |
| 1b | zika_77 | CcaCas13b | tttgcaccatccatctcagcctcca | tttgcaccat | GTTGGAACTGCT | Zika | 1b |
| | | | gagctGTTGGAACTGCTCTCATTTT | ccatctcagc | CTCATTTTGGAGG | ssRNA | |
| | | | GGAGGGTAATCACAAC | ctccagagct | GTAATCACAAC | | |
| | | | (SEQ ID. NO: 1473) | (SEQ ID. | (SEQ ID. | | |
| | | | | NO: | NO: 1475) | | |
| | | | | 1474) | | | |
| 1b | zika_78 | CcaCas13b | ctttgcaccatccatctcagcctcc | ctttgcacca | GTTGGAACTGCT | Zika | 1b |
| | | | agagcGTTGGAACTGCTCTCATTTT | tccatctcag | CTCATTTTGGAGG | ssRNA | |
| | | | GGAGGGTAATCACAAC | cctccagag | GTAATCACAAC | | |
| | | | (SEQ ID. NO: 1476) | c (SEQ | (SEQ ID. | | |
| | | | | ID. NO: | NO: 1478) | | |
| | | | | 1477) | | | |
| 1b | zika_79 | CcaCas13b | cctttgcaccatccatctcagcctc | cctttgcacc | GTTGGAACTGCT | Zika | 1b |
| | | | cagagGTTGGAACTGCTCTCATTTT | atccatctca | CTCATTTTGGAGG | ssRNA | |
| | | | GGAGGGTAATCACAAC | gcctccaga | GTAATCACAAC | | |
| | | | (SEQ ID. NO: 1479) | g (SEQ | (SEQ ID. | | |
| | | | | ID. NO: | NO: 1481) | | |
| | | | | 1480) | | | |
| 1b | zika_80 | CcaCas13b | ccctttgcaccatccatctcagcct | ccctttgcac | GTTGGAACTGCT | Zika | 1b |
| | | | ccagaGTTGGAACTGCTCTCATTTT | catccatctc | CTCATTTTGGAGG | ssRNA | |
| | | | GGAGGGTAATCACAAC | agcctccag | GTAATCACAAC | | |
| | | | (SEQ ID. NO: 1482) | a (SEQ | (SEQ ID. | | |
| | | | | ID. NO: | NO: 1484) | | |
| | | | | 1483) | | | |
| 1b | zika_81 | CcaCas13b | tccctttgcaccatccatctcagcc | tccctttgca | GTTGGAACTGCT | Zika | 1b |
| | | | tccagGTTGGAACTGCTCTCATTTT | ccatccatct | CTCATTTTGGAGG | ssRNA | |
| | | | GGAGGGTAATCACAAC | cagcctcca | GTAATCACAAC | | |
| | | | (SEQ ID. NO: 1485) | g (SEQ | (SEQ ID. | | |
| | | | | ID. NO: | NO: 1487) | | |
| | | | | 1486) | | | |
| 1b | zika_82 | CcaCas13b | ttccctttgcaccatccatctcagc | ttccctttgca | GTTGGAACTGCT | Zika | 1b |
| | | | ctccaGTTGGAACTGCTCTCATTTT | ccatccatct | CTCATTTTGGAGG | ssRNA | |
| | | | GGAGGGTAATCACAAC | cagcctcca | GTAATCACAAC | | |
| | | | (SEQ ID. NO: 1488) | (SEQ ID. | (SEQ ID. | | |
| | | | | NO: | NO: 1490) | | |
| | | | | 1489) | | | |
| 1b | zika_83 | CcaCas13b | cttccctttgcaccatccatctcag | cttccctttgc | GTTGGAACTGCT | Zika | 1b |
| | | | cctccGTTGGAACTGCTCTCATTTT | accatccatc | CTCATTTTGGAGG | ssRNA | |
| | | | GGAGGGTAATCACAAC | tcagcctcc | GTAATCACAAC | | |
| | | | (SEQ ID. NO: 1491) | (SEQ ID. | (SEQ ID. | | |
| | | | | NO: | NO: 1493) | | |
| | | | | 1492) | | | |
| 1b | zika_84 | CcaCas13b | ccttccctttgcaccatccatctca | ccttccctttg | GTTGGAACTGCT | Zika | 1b |
| | | | gcctcGTTGGAACTGCTCTCATTTT | caccatccat | CTCATTTTGGAGG | ssRNA | |
| | | | GGAGGGTAATCACAAC | ctcagcctc | GTAATCACAAC | | |
| | | | (SEQ ID. NO: 1494) | (SEQ ID. | (SEQ ID. | | |
| | | | | NO: | NO: 1496) | | |
| | | | | 1495) | | | |
| 1b | zika_85 | CcaCas13b | gccttccctttgcaccatccatctc | gccttccctt | GTTGGAACTGCT | Zika | 1b |
| | | | agcctGTTGGAACTGCTCTCATTTT | tgcaccatcc | CTCATTTTGGAGG | ssRNA | |
| | | | GGAGGGTAATCACAAC | atctcagcct | GTAATCACAAC | | |
| | | | (SEQ ID. NO: 1497) | (SEQ ID. | (SEQ ID. | | |
| | | | | NO: | NO: 1499) | | |
| | | | | 1498) | | | |
| 1b | zika_86 | CcaCas13b | agccttccctttgcaccatccatct | agccttccct | GTTGGAACTGCT | Zika | 1b |
| | | | cagccGTTGGAACTGCTCTCATTTT | ttgcaccatc | CTCATTTTGGAGG | ssRNA | |
| | | | GGAGGGTAATCACAAC | catctcagcc | GTAATCACAAC | | |
| | | | (SEQ ID. NO: 1500) | (SEQ ID. | (SEQ ID. | | |
| | | | | NO: | NO: 1502) | | |
| | | | | 1501) | | | |
| 1b | zika_87 | CcaCas13b | cagccttccctttgcaccatccatc | cagccttccc | GTTGGAACTGCT | Zika | 1b |
| | | | tcagcGTTGGAACTGCTCTCATTTT | tttgcaccat | CTCATTTTGGAGG | ssRNA | |
| | | | GGAGGGTAATCACAAC | ccatctcagc | GTAATCACAAC | | |
| | | | (SEQ ID. NO: 1503) | (SEQ ID. | (SEQ ID. | | |
| | | | | NO: | NO: 1505) | | |
| | | | | 1504) | | | |
| 1b | zika_88 | CcaCas13b | acagccttccctttgcaccatccat | acagccttcc | GTTGGAACTGCT | Zika | 1b |
| | | | ctcagGTTGGAACTGCTCTCATTTT | ctttgcacca | CTCATTTTGGAGG | ssRNA | |
| | | | GGAGGGTAATCACAAC | tccatctcag | GTAATCACAAC | | |
| | | | (SEQ ID. NO: 1506) | (SEQ ID. | (SEQ ID. | | |
| | | | | NO: | NO: 1508) | | |
| | | | | 1507) | | | |
| 1b | zika_89 | CcaCas13b | gacagccttccctttgcaccatcca | gacagccttc | GTTGGAACTGCT | Zika | 1b |
| | | | tctcaGTTGGAACTGCTCTCATTTT | cctttgcacc | CTCATTTTGGAGG | ssRNA | |
| | | | GGAGGGTAATCACAAC | atccatctca | GTAATCACAAC | | |
| | | | (SEQ ID. NO: 1509) | (SEQ ID. | (SEQ ID. | | |
| | | | | NO: | NO: 1511) | | |
| | | | | 1510) | | | |
| 1b | zika_90 | CcaCas13b | ggacagccttccctttgcaccatcc | ggacagcct | GTTGGAACTGCT | Zika | 1b |
| | | | atctcGTTGGAACTGCTCTCATTTT | tccctttgca | CTCATTTTGGAGG | ssRNA | |
| | | | GGAGGGTAATCACAAC | ccatccatct | GTAATCACAAC | | |
| | | | (SEQ ID. NO: 1512) | c (SEQ | (SEQ ID. | | |
| | | | | ID. NO: | NO: 1514) | | |
| | | | | 1513) | | | |
| 1b | zika_91 | CcaCas13b | aggacagccttccctttgcaccatc | aggacagcc | GTTGGAACTGCT | Zika | 1b |
| | | | catctGTTGGAACTGCTCTCATTTT | ttccctttgca | CTCATTTTGGAGG | ssRNA | |
| | | | GGAGGGTAATCACAAC | ccatccatct | GTAATCACAAC | | |
| | | | (SEQ ID. NO: 1515) | (SEQ ID. | (SEQ ID. | | |
| | | | | NO: | NO: 1517) | | |
| | | | | 1516) | | | |
| 1b | zika_92 | CcaCas13b | gaggacagccttccctttgcaccat | gaggacagc | GTTGGAACTGCT | Zika | 1b |
| | | | ccatcGTTGGAACTGCTCTCATTTT | cttccctttgc | CTCATTTTGGAGG | ssRNA | |
| | | | GGAGGGTAATCACAAC | accatccatc | GTAATCACAAC | | |
| | | | (SEQ ID. NO: 1518) | (SEQ ID. | (SEQ ID. | | |
| | | | | NO: | NO: 1520) | | |
| | | | | 1519) | | | |
| 1b | zika_93 | CcaCas13b | agaggacagccttccctttgcacca | agaggacag | GTTGGAACTGCT | Zika | 1b |
| | | | tccatGTTGGAACTGCTCTCATTTT | ccttccctttg | CTCATTTTGGAGG | ssRNA | |
| | | | GGAGGGTAATCACAAC | caccatccat | GTAATCACAAC | | |
| | | | (SEQ ID. NO: 1521) | (SEQ ID. | (SEQ ID. | | |
| | | | | NO: | NO: 1523) | | |
| | | | | 1522) | | | |
| 1b | zika_94 | CcaCas13b | cagaggacagccttccctttgcacc | cagaggaca | GTTGGAACTGCT | Zika | 1b |
| | | | atccaGTTGGAACTGCTCTCATTTT | gccttcccttt | CTCATTTTGGAGG | ssRNA | |
| | | | GGAGGGTAATCACAAC | gcaccatcc | GTAATCACAAC | | |
| | | | (SEQ ID. NO: 1524) | a (SEQ | (SEQ ID. | | |
| | | | | ID. NO: | NO: 1526) | | |
| | | | | 1525) | | | |
| 7a | dengue_0 | CcaCas13b | tgttgagaggttggcccctgaatat | tgttgagagg | GTTGGAACTGCT | Dengue | 7a |
| | | | gtactGTTGGAACTGCTCTCATTTT | ttggcccctg | CTCATTTTGGAGG | ssRNA | |
| | | | GGAGGGTAATCACAAC | aatatgtact | GTAATCACAAC | | |
| | | | (SEQ ID. NO: 1527) | (SEQ ID. | (SEQ ID. | | |
| | | | | NO: | NO: 1529) | | |
| | | | | 1528) | | | |
| 7a | dengue_1 | CcaCas13b | ttgttgagaggttggcccctgaata | ttgttgagag | GTTGGAACTGCT | Dengue | 7a |
| | | | gttacGTTGGAACTGCTCTCATTTT | gttggcccct | CTCATTTTGGAGG | ssRNA | |
| | | | GGAGGGTAATCACAAC | gaatatgtac | GTAATCACAAC | | |
| | | | (SEQ ID. NO: 1530) | (SEQ ID. | (SEQ ID. | | |
| | | | | NO: | NO: 1532) | | |
| | | | | 1531) | | | |
| 7a | dengue_2 | CcaCas13b | attgttgagaggttggcccctgaat | attgttgaga | GTTGGAACTGCT | Dengue | 7a |
| | | | atgtaGTTGGAACTGCTCTCATTTT | ggttggccc | CTCATTTTGGAGG | ssRNA | |
| | | | GGAGGGTAATCACAAC | ctgaatatgt | GTAATCACAAC | | |
| | | | (SEQ ID. NO: 1533) | a (SEQ | (SEQ ID. | | |
| | | | | ID. NO: | NO: 1535) | | |
| | | | | 1534) | | | |
| 7a | dengue_3 | CcaCas13b | cattgttgagaggttggcccctgaa | cattgttgag | GTTGGAACTGCT | Dengue | 7a |
| | | | tatgtGTTGGAACTGCTCTCATTTT | aggttggcc | CTCATTTTGGAGG | ssRNA | |
| | | | GGAGGGTAATCACAAC | cctgaatatg | GTAATCACAAC | | |
| | | | (SEQ ID. NO: 1536) | t (SEQ | (SEQ ID. | | |
| | | | | ID. NO: | NO: 1538) | | |
| | | | | 1537) | | | |
| 7a | dengue_4 | CcaCas13b | tcattgttgagaggttggcccctga | tcattgttgag | GTTGGAACTGCT | Dengue | 7a |
| | | | atatgGTTGGAACTGCTCTCATTTT | aggttggcc | CTCATTTTGGAGG | ssRNA | |
| | | | GGAGGGTAATCACAAC | cctgaatatg | GTAATCACAAC | | |
| | | | (SEQ ID. NO: 1539) | (SEQ ID. | (SEQ ID. | | |
| | | | | NO: | NO: 1541) | | |
| | | | | 1540) | | | |
| 7a | dengue_5 | CcaCas13b | gtcattgttgagaggttggcccctg | gtcattgttga | GTTGGAACTGCT | Dengue | 7a |
| | | | aatatGTTGGAACTGCTCTCATTTT | gaggttggc | CTCATTTTGGAGG | ssRNA | |
| | | | GGAGGGTAATCACAAC | ccctgaatat | GTAATCACAAC | | |
| | | | (SEQ ID. NO: 1542) | (SEQ ID. | (SEQ ID. | | |
| | | | | NO: | NO: 1544) | | |
| | | | | 1543) | | | |
| 7a | dengue_6 | CcaCas13b | cgtcattgttgagaggttggcccct | cgtcattgttg | GTTGGAACTGCT | Dengue | 7a |
| | | | gaataGTTGGAACTGCTCTCATTTT | agaggttgg | CTCATTTTGGAGG | ssRNA | |
| | | | GGAGGGTAATCACAAC | cccctgaata | GTAATCACAAC | | |
| | | | (SEQ ID. NO: 1545) | (SEQ ID. | (SEQ ID. | | |
| | | | | NO: | NO: 1547) | | |
| | | | | 1546) | | | |
| 7a | dengue_7 | CcaCas13b | tcgtcattgttgagaggttggcccc | tcgtcattgtt | GTTGGAACTGCT | Dengue | 7a |
| | | | tgaatGTTGGAACTGCTCTCATTTT | gagaggttg | CTCATTTTGGAGG | ssRNA | |
| | | | GGAGGGTAATCACAAC | gcccctgaat | GTAATCACAAC | | |
| | | | (SEQ ID. NO: 1548) | (SEQ ID. | (SEQ ID. | | |
| | | | | NO: | NO: 1550) | | |
| | | | | 1549) | | | |
| 7a | dengue_8 | CcaCas13b | ttcgtcattgttgagaggttggccc | ttcgtcattgt | GTTGGAACTGCT | Dengue | 7a |
| | | | ctgaaGTTGGAACTGCTCTCATTTT | tgagaggttg | CTCATTTTGGAGG | ssRNA | |
| | | | GGAGGGTAATCACAAC | gcccctgaa | GTAATCACAAC | | |
| | | | (SEQ ID. NO: 1551) | (SEQ ID. | (SEQ ID. | | |
| | | | | NO: | NO: 1553) | | |
| | | | | 1552) | | | |
| 7a | dengue_9 | CcaCas13b | cttcgtcattgttgagaggttggcc | cttcgtcattg | GTTGGAACTGCT | Dengue | 7a |
| | | | cctgaGTTGGAACTGCTCTCATTTT | ttgagaggtt | CTCATTTTGGAGG | ssRNA | |
| | | | GGAGGGTAATCACAAC | ggcccctga | GTAATCACAAC | | |
| | | | (SEQ ID. NO: 1554) | (SEQ ID. | (SEQ ID. | | |
| | | | | NO: | NO: 1556) | | |
| | | | | 1555) | | | |
| 7a | dengue_1 | CcaCas13b | tcttcgtcattgttgagaggttggc | tcttcgtcatt | GTTGGAACTGCT | Dengue | 7a |
| | 0 | | ccctgGTTGGAACTGCTCTCATTTT | gttgagaggt | CTCATTTTGGAGG | ssRNA | |
| | | | GGAGGGTAATCACAAC | tggcccctg | GTAATCACAAC | | |
| | | | (SEQ ID. NO: 1557) | (SEQ ID. | (SEQ ID. | | |
| | | | | NO: | NO: 1559) | | |
| | | | | 1558) | | | |
| 7a | dengue_1 | CcaCas13b | gtcttcgtcattgttgagaggttgg | gtcttcgtcat | GTTGGAACTGCT | Dengue | 7a |
| | 1 | | cccctGTTGGAACTGCTCTCATTTT | tgttgagagg | CTCATTTTGGAGG | ssRNA | |
| | | | GGAGGGTAATCACAAC | ttggcccct | GTAATCACAAC | | |
| | | | (SEQ ID. NO: 1560) | (SEQ ID. | (SEQ ID. | | |
| | | | | NO: | NO: 1562) | | |
| | | | | 1561) | | | |
| 7a | dengue_1 | CcaCas13b | ggtcttcgtcattgttgagaggttg | ggtcttcgtc | GTTGGAACTGCT | Dengue | 7a |
| | 2 | | gccccGTTGGAACTGCTCTCATTTT | attgttgaga | CTCATTTTGGAGG | ssRNA | |
| | | | GGAGGGTAATCACAAC | ggttggccc | GTAATCACAAC | | |
| | | | (SEQ ID. NO: 1563) | c (SEQ | (SEQ ID. | | |
| | | | | ID. NO: | NO: 1565) | | |
| | | | | 1564) | | | |
| 7a | dengue_1 | CcaCas13b | tggtcttcgtcattgttgagaggtt | tggtcttcgtc | GTTGGAACTGCT | Dengue | 7a |
| | 3 | | ggcccGTTGGAACTGCTCTCATTTT | attgttgaga | CTCATTTTGGAGG | ssRNA | |
| | | | GGAGGGTAATCACAAC | ggttggccc | GTAATCACAAC | | |
| | | | (SEQ ID. NO: 1566) | (SEQ ID. | (SEQ ID. | | |
| | | | | NO: | NO: 1568) | | |
| | | | | 1567) | | | |
| 7a | dengue_1 | CcaCas13b | atggtcttcgtcattgttgagaggt | atggtcttcgt | GTTGGAACTGCT | Dengue | 7a |
| | 4 | | tggccGTTGGAACTGCTCTCATTTT | cattgttgag | CTCATTTTGGAGG | ssRNA | |
| | | | GGAGGGTAATCACAAC | aggttggcc | GTAATCACAAC | | |
| | | | (SEQ ID. NO: 1569) | (SEQ ID. | (SEQ ID. | | |
| | | | | NO: | NO: 1571) | | |
| | | | | 1570) | | | |
| 7a | dengue_1 | CcaCas13b | catggtcttcgtcattgttgagagg | catggtcttc | GTTGGAACTGCT | Dengue | 7a |
| | 5 | | ttggcGTTGGAACTGCTCTCATTTT | gtcattgttga | CTCATTTTGGAGG | ssRNA | |
| | | | GGAGGGTAATCACAAC | gaggttggc | GTAATCACAAC | | |
| | | | (SEQ ID. NO: 1572) | (SEQ ID. | (SEQ ID. | | |
| | | | | NO: | NO: 1574) | | |
| | | | | 1573) | | | |
| 7a | dengue_1 | CcaCas13b | gcatggtcttcgtcattgttgagag | gcatggtctt | GTTGGAACTGCT | Dengue | 7a |
| | 6 | | gttggGTTGGAACTGCTCTCATTTT | cgtcattgttg | CTCATTTTGGAGG | ssRNA | |
| | | | GGAGGGTAATCACAAC | agaggttgg | GTAATCACAAC | | |
| | | | (SEQ ID. NO: 1575) | (SEQ ID. | (SEQ ID. | | |
| | | | | NO: | NO: 1577) | | |
| | | | | 1576) | | | |
| 7a | dengue_1 | CcaCas13b | agcatggtcttcgtcattgttgaga | agcatggtct | GTTGGAACTGCT | Dengue | 7a |
| | 7 | | ggttgGTTGGAACTGCTCTCATTTT | tcgtcattgtt | CTCATTTTGGAGG | ssRNA | |
| | | | GGAGGGTAATCACAAC | gagaggttg | GTAATCACAAC | | |
| | | | (SEQ ID. NO: 1578) | (SEQ ID. | (SEQ ID. | | |
| | | | | NO: | NO: 1580) | | |
| | | | | 1579) | | | |
| 7a | dengue_1 | CcaCas13b | gagcatggtcttcgtcattgttgag | gagcatggt | GTTGGAACTGCT | Dengue | 7a |
| | 8 | | aggttGTTGGAACTGCTCTCATTTT | cttcgtcattg | CTCATTTTGGAGG | ssRNA | |
| | | | GGAGGGTAATCACAAC | ttgagaggtt | GTAATCACAAC | | |
| | | | (SEQ ID. NO: 1581) | (SEQ ID. | (SEQ ID. | | |
| | | | | NO: | NO: 1583) | | |
| | | | | 1582) | | | |
| 7a | dengue_1 | CcaCas13b | tgagcatggtcttcgtcattgttga | tgagcatggt | GTTGGAACTGCT | Dengue | 7a |
| | 9 | | gaggtGTTGGAACTGCTCTCATTTT | cttcgtcattg | CTCATTTTGGAGG | ssRNA | |
| | | | GGAGGGTAATCACAAC | ttgagaggt | GTAATCACAAC | | |
| | | | (SEQ ID. NO: 1584) | (SEQ ID. | (SEQ ID. | | |
| | | | | NO: | NO: 1586) | | |
| | | | | 1585) | | | |
| 7a | dengue_2 | CcaCas13b | agtgagcatggtcttcgtcattgtt | agtgagcat | GTTGGAACTGCT | Dengue | 7a |
| | 0 | | gagagGTTGGAACTGCTCTCATTTT | ggtcttcgtc | CTCATTTTGGAGG | ssRNA | |
| | | | GGAGGGTAATCACAAC | attgttgaga | GTAATCACAAC | | |
| | | | (SEQ ID. NO: 1587) | g (SEQ | (SEQ ID. | | |
| | | | | ID. NO: | NO: 1589) | | |
| | | | | 1588) | | | |
| 7a | dengue_2 | CcaCas13b | ccagtgagcatggtcttcgtcattg | ccagtgagc | GTTGGAACTGCT | Dengue | 7a |
| | 1 | | ttgagGTTGGAACTGCTCTCATTTT | atggtcttcgt | CTCATTTTGGAGG | ssRNA | |
| | | | GGAGGGTAATCACAAC | cattgttgag | GTAATCACAAC | | |
| | | | (SEQ ID. NO: 1590) | (SEQ ID. | (SEQ ID. | | |
| | | | | NO: | NO: 1592) | | |
| | | | | 1591) | | | |
| 7a | dengue_2 | CcaCas13b | gtccagtgagcatggtcttcgtcat | gtccagtga | GTTGGAACTGCT | Dengue | 7a |
| | 2 | | tgttgGTTGGAACTGCTCTCATTTT | gcatggtctt | CTCATTTTGGAGG | ssRNA | |
| | | | GGAGGGTAATCACAAC | cgtcattgttg | GTAATCACAAC | | |
| | | | (SEQ ID. NO: 1593) | (SEQ ID. | (SEQ ID. | | |
| | | | | NO: | NO: 1595) | | |
| | | | | 1594) | | | |
| 7a | dengue_2 | CcaCas13b | ctgtccagtgagcatggtcttcgtc | ctgtccagtg | GTTGGAACTGCT | Dengue | 7a |
| | 3 | | attgtGTTGGAACTGCTCTCATTTT | agcatggtct | CTCATTTTGGAGG | ssRNA | |
| | | | GGAGGGTAATCACAAC | tcgtcattgt | GTAATCACAAC | | |
| | | | (SEQ ID. NO: 1596) | (SEQ ID. | (SEQ ID. | | |
| | | | | NO: | NO: 1598) | | |
| | | | | 1597) | | | |
| 7a | dengue_2 | CcaCas13b | ttctgtccagtgagcatggtcttcg | ttctgtccagt | GTTGGAACTGCT | Dengue | 7a |
| | 4 | | tcattGTTGGAACTGCTCTCATTTT | gagcatggt | CTCATTTTGGAGG | ssRNA | |
| | | | GGAGGGTAATCACAAC | cttcgtcatt | GTAATCACAAC | | |
| | | | (SEQ ID. NO: 1599) | (SEQ ID. | (SEQ ID. | | |
| | | | | NO: | NO: 1601) | | |
| | | | | 1600) | | | |
| 7a | dengue_2 | CcaCas13b | gcttctgtccagtgagcatggtctt | gcttctgtcc | GTTGGAACTGCT | Dengue | 7a |
| | 5 | | cgtcaGTTGGAACTGCTCTCATTTT | agtgagcat | CTCATTTTGGAGG | ssRNA | |
| | | | GGAGGGTAATCACAAC | ggtcttcgtc | GTAATCACAAC | | |
| | | | (SEQ ID. NO: 1602) | a (SEQ | (SEQ ID. | | |
| | | | | ID. NO: | NO: 1604) | | |
| | | | | 1603) | | | |
| 7a | dengue_2 | CcaCas13b | ttgcttctgtccagtgagcatggtc | ttgcttctgtc | GTTGGAACTGCT | Dengue | 7a |
| | 6 | | ttcgtGTTGGAACTGCTCTCATTTT | cagtgagca | CTCATTTTGGAGG | ssRNA | |
| | | | GGAGGGTAATCACAAC | tggtcttcgt | GTAATCACAAC | | |
| | | | (SEQ ID. NO: 1605) | (SEQ ID. | (SEQ ID. | | |
| | | | | NO: | NO: 1607) | | |
| | | | | 1606) | | | |
| 7a | dengue_2 | CcaCas13b | ttttgcttctgtccagtgagcatgg | ttttgcttctg | GTTGGAACTGCT | Dengue | 7a |
| | 7 | | tTGGAACTGCTCTCATTTTG | tccagtgagc | CTCATTTTGGAGG | ssRNA | |
| | | | cttcGTGAGGGTAATCACAAC | atggtcttc | GTAATCACAAC | | |
| | | | (SEQ ID. NO: 1608) | (SEQ ID. | (SEQ ID. | | |
| | | | | NO: | NO: 1610) | | |
| | | | | 1609) | | | |
| 7a | dengue_2 | CcaCas13b | atttttgcttctgtccagtgagcat | atttttgcttc | GTTGGAACTGCT | Dengue | 7a |
| | 8 | | ggtctGTTGGAACTGCTCTCATTTT | tgtccagtga | CTCATTTTGGAGG | ssRNA | |
| | | | GGAGGGTAATCACAAC | gcatggtct | GTAATCACAAC | | |
| | | | (SEQ ID. NO: 1611) | (SEQ ID. | (SEQ ID. | | |
| | | | | NO: | NO: 1613) | | |
| | | | | 1612) | | | |
| 7a | dengue_2 | CcaCas13b | gcatttttgcttctgtccagtgagc | gcatttttgct | GTTGGAACTGCT | Dengue | 7a |
| | 9 | | atggtGTTGGAACTGCTCTCATTTT | tctgtccagt | CTCATTTTGGAGG | ssRNA | |
| | | | GGAGGGTAATCACAAC | gagcatggt | GTAATCACAAC | | |
| | | | (SEQ ID. NO: 1614) | (SEQ ID. | (SEQ ID. | | |
| | | | | NO: | NO: 1616) | | |
| | | | | 1615) | | | |
| 7a | dengue_3 | CcaCas13b | cagcatttttgcttctgtccagtga | cagcatttttg | GTTGGAACTGCT | Dengue | 7a |
| | 0 | | gcatgGTTGGAACTGCTCTCATTTT | cttctgtcca | CTCATTTTGGAGG | ssRNA | |
| | | | GGAGGGTAATCACAAC | gtgagcatg | GTAATCACAAC | | |
| | | | (SEQ ID. NO: 1617) | (SEQ ID. | (SEQ ID. | | |
| | | | | NO: | NO: 1619) | | |
| | | | | 1618) | | | |
| 7a | dengue_3 | CcaCas13b | agcagcatttttgcttctgtccagt | agcagcattt | GTTGGAACTGCT | Dengue | 7a |
| | 1 | | gagcaGTTGGAACTGCTCTCATTTT | ttgcttctgtc | CTCATTTTGGAGG | ssRNA | |
| | | | GGAGGGTAATCACAAC | cagtgagca | GTAATCACAAC | | |
| | | | (SEQ ID. NO: 1620) | (SEQ ID. | (SEQ ID. | | |
| | | | | NO: | NO: 1622) | | |
| | | | | 1621) | | | |
| 7a | dengue_3 | CcaCas13b | ccagcagcatttttgcttctgtcca | ccagcagca | GTTGGAACTGCT | Dengue | 7a |
| | 2 | | gtgagGTTGGAACTGCTCTCATTTT | tttttgcttct | CTCATTTTGGAGG | ssRNA | |
| | | | GGAGGGTAATCACAAC | gtccagtgag | GTAATCACAAC | | |
| | | | (SEQ ID. NO: 1623) | (SEQ ID. | (SEQ ID. | | |
| | | | | NO: | NO: 1625) | | |
| | | | | 1624) | | | |
| 7a | dengue_3 | CcaCas13b | gtccagcagcatttttgcttctgtc | gtccagcag | GTTGGAACTGCT | Dengue | 7a |
| | 3 | | cagtgGTTGGAACTGCTCTCATTTT | catttttgctt | CTCATTTTGGAGG | ssRNA | |
| | | | GGAGGGTAATCACAAC | ctgtccagtg | GTAATCACAAC | | |
| | | | (SEQ ID. NO: 1626) | (SEQ ID. | (SEQ ID. | | |
| | | | | NO: | NO: 1628) | | |
| | | | | 1627) | | | |
| 7a | dengue_3 | CcaCas13b | ttgtccagcagcatttttgcttctg | ttgtccagca | GTTGGAACTGCT | Dengue | 7a |
| | 4 | | tTccagGTGGAACTGCTCTCATTTT | gcatttttgct | CTCATTTTGGAGG | ssRNA | |
| | | | GGAGGGTAATCACAAC | tctgtccag | GTAATCACAAC | | |
| | | | (SEQ ID. NO: 1629) | (SEQ ID. | (SEQ ID. | | |
| | | | | NO: | NO: 1631) | | |
| | | | | 1630) | | | |
| 7a | dengue_3 | CcaCas13b | tgttgtccagcagcatttttgcttc | tgttgtccag | GTTGGAACTGCT | Dengue | 7a |
| | 5 | | tgtccGTTGGAACTGCTCTCATTTT | cagcatttttg | CTCATTTTGGAGG | ssRNA | |
| | | | GGAGGGTAATCACAAC | cttctgtcc | GTAATCACAAC | | |
| | | | (SEQ ID. NO: 1632) | (SEQ ID. | (SEQ ID. | | |
| | | | | NO: | NO: 1634) | | |
| | | | | 1633) | | | |
| 7a | dengue_3 | CcaCas13b | gatgttgtccagcagcatttttgct | gatgttgtcc | GTTGGAACTGCT | Dengue | 7a |
| | 6 | | tctgtGTTGGAACTGCTCTCATTTT | agcagcattt | CTCATTTTGGAGG | ssRNA | |
| | | | GGAGGGTAATCACAAC | ttgettctgt | GTAATCACAAC | | |
| | | | (SEQ ID. NO: 1635) | (SEQ ID. | (SEQ ID. | | |
| | | | | NO: | NO: 1637) | | |
| | | | | 1636) | | | |
| 7a | dengue_3 | CcaCas13b | ttgatgttgtccagcagcatttttg | ttgatgttgtc | GTTGGAACTGCT | Dengue | 7a |
| | 7 | | cttctGTTGGAACTGCTCTCATTTT | cagcagcatt | CTCATTTTGGAGG | ssRNA | |
| | | | GGAGGGTAATCACAAC | tttgettct | GTAATCACAAC | | |
| | | | (SEQ ID. NO: 1638) | (SEQ ID. | (SEQ ID. | | |
| | | | | NO: | NO: 1640) | | |
| | | | | 1639) | | | |
| 7a | dengue_3 | CcaCas13b | tgttgatgttgtccagcagcatttt | tgttgatgttg | GTTGGAACTGCT | Dengue | 7a |
| | 8 | | tgcttGTTGGAACTGCTCTCATTTT | tccagcagc | CTCATTTTGGAGG | ssRNA | |
| | | | GGAGGGTAATCACAAC | atttttgctt | GTAATCACAAC | | |
| | | | (SEQ ID. NO: 1641) | (SEQ ID. | (SEQ ID. | | |
| | | | | NO: | NO: 1643) | | |
| | | | | 1642) | | | |
| 7a | dengue_3 | CcaCas13b | tgtgttgatgttgtccagcagcatt | tgtgttgatgt | GTTGGAACTGCT | Dengue | 7a |
| | 9 | | tttgcGTTGGAACTGCTCTCATTTT | tgtccagca | CTCATTTTGGAGG | ssRNA | |
| | | | GGAGGGTAATCACAAC | gcatttttgc | GTAATCACAAC | | |
| | | | (SEQ ID. NO: 1644) | (SEQ ID. | (SEQ ID. | | |
| | | | | NO: | NO: 1646) | | |
| | | | | 1645) | | | |
| 7a | dengue_4 | CcaCas13b | ggtgtgttgatgttgtccagcagca | ggtgtgttga | GTTGGAACTGCT | Dengue | 7a |
| | 0 | | tttttGTTGGAACTGCTCTCATTTT | tgttgtccag | CTCATTTTGGAGG | ssRNA | |
| | | | GGAGGGTAATCACAAC | cagcattttt | GTAATCACAAC | | |
| | | | (SEQ ID. NO: 1647) | (SEQ ID. | (SEQ ID. | | |
| | | | | NO: | NO: 1649) | | |
| | | | | 1648) | | | |
| 7a | dengue_4 | CcaCas13b | ctggtgtgttgatgttgtccagcag | ctggtgtgtt | GTTGGAACTGCT | Dengue | 7a |
| | 1 | | catttGTTGGAACTGCTCTCATTTT | gatgttgtcc | CTCATTTTGGAGG | ssRNA | |
| | | | GGAGGGTAATCACAAC | agcagcattt | GTAATCACAAC | | |
| | | | (SEQ ID. NO: 1650) | (SEQ ID. | (SEQ ID. | | |
| | | | | NO: | NO: 1652) | | |
| | | | | 1651) | | | |
| 7a | dengue_4 | CcaCas13b | ttctggtgtgttgatgttgtccagc | ttctggtgtgt | GTTGGAACTGCT | Dengue | 7a |
| | 2 | | agcatGTTGGAACTGCTCTCATTTT | tgatgttgtcc | CTCATTTTGGAGG | ssRNA | |
| | | | GGAGGGTAATCACAAC | agcagcat | GTAATCACAAC | | |
| | | | (SEQ ID. NO: 1653) | (SEQ ID. | (SEQ ID. | | |
| | | | | NO: | NO: 1655) | | |
| | | | | 1654) | | | |
| 7a | dengue_4 | CcaCas13b | ccttctggtgtgttgatgttgtcca | ccttctggtgt | GTTGGAACTGCT | Dengue | 7a |
| | 3 | | gcagcGTTGGAACTGCTCTCATTTT | gttgatgttgt | CTCATTTTGGAGG | ssRNA | |
| | | | GGAGGGTAATCACAAC | ccagcagc | GTAATCACAAC | | |
| | | | (SEQ ID. NO: 1656) | (SEQ ID. | (SEQ ID. | | |
| | | | | NO: | NO: 1658) | | |
| | | | | 1657) | | | |
| 7a | dengue_4 | CcaCas13b | tcccttctggtgtgttgatgttgtc | tcccttctggt | GTTGGAACTGCT | Dengue | 7a |
| | 4 | | cagcaGTTGGAACTGCTCTCATTTT | gtgttgatgtt | CTCATTTTGGAGG | ssRNA | |
| | | | GGAGGGTAATCACAAC | gtccagca | GTAATCACAAC | | |
| | | | (SEQ ID. NO: 1659) | (SEQ ID. | (SEQ ID. | | |
| | | | | NO: | NO: 1661) | | |
| | | | | 1660) | | | |
| 7a | dengue_4 | CcaCas13b | aatcccttctggtgtgttgatgttg | aatcccttct | GTTGGAACTGCT | Dengue | 7a |
| | 5 | | tccagGTTGGAACTGCTCTCATTTT | ggtgtgttga | CTCATTTTGGAGG | ssRNA | |
| | | | GGAGGGTAATCACAAC | tgttgtccag | GTAATCACAAC | | |
| | | | (SEQ ID. NO: 1662) | (SEQ ID. | (SEQ ID. | | |
| | | | | NO: | NO: 1664) | | |
| | | | | 1663) | | | |
| 7a | dengue_4 | CcaCas13b | ataatcccttctggtgtgttgatgt | ataatcccttc | GTTGGAACTGCT | Dengue | 7a |
| | 6 | | tgtccGTTGGAACTGCTCTCATTTT | tggtgtgttg | CTCATTTTGGAGG | ssRNA | |
| | | | GGAGGGTAATCACAAC | atgttgtcc | GTAATCACAAC | | |
| | | | (SEQ ID. NO: 1665) | (SEQ ID. | (SEQ ID. | | |
| | | | | NO: | NO: 1667) | | |
| | | | | 1666) | | | |
| 7a | dengue_4 | CcaCas13b | gtataatcccttctggtgtgttgat | gtataatccc | GTTGGAACTGCT | Dengue | 7a |
| | 7 | | gttgtGTTGGAACTGCTCTCATTTT | ttctggtgtgt | CTCATTTTGGAGG | ssRNA | |
| | | | GGAGGGTAATCACAAC | tgatgttgt | GTAATCACAAC | | |
| | | | (SEQ ID. NO: 1668) | (SEQ ID. | (SEQ ID. | | |
| | | | | NO: | NO: 1670) | | |
| | | | | 1669) | | | |
| 7a | dengue_4 | CcaCas13b | tggtataatcccttctggtgtgttg | tggtataatc | GTTGGAACTGCT | Dengue | 7a |
| | 8 | | atgttGTTGGAACTGCTCTCATTTT | ccttctggtgt | CTCATTTTGGAGG | ssRNA | |
| | | | GGAGGGTAATCACAAC | gttgatgtt | GTAATCACAAC | | |
| | | | (SEQ ID. NO: 1671) | (SEQ ID. | (SEQ ID. | | |
| | | | | NO: | NO: 1673) | | |
| | | | | 1672) | | | |
| 7a | dengue_4 | CcaCas13b | gctggtataatcccttctggtgtgt | gctggtataa | GTTGGAACTGCT | Dengue | 7a |
| | 9 | | tgatgGTTGGAACTGCTCTCATTTT | tcccttctggt | CTCATTTTGGAGG | ssRNA | |
| | | | GGAGGGTAATCACAAC | gtgttgatg | GTAATCACAAC | | |
| | | | (SEQ ID. NO: 1674) | (SEQ ID. | (SEQ ID. | | |
| | | | | NO: | NO: 1676) | | |
| | | | | 1675) | | | |
| 7a | dengue_5 | CcaCas13b | gagctggtataatcccttctggtgt | gagctggtat | GTTGGAACTGCT | Dengue | 7a |
| | 0 | | gttgaGTTGGAACTGCTCTCATTTT | aatcccttct | CTCATTTTGGAGG | ssRNA | |
| | | | GGAGGGTAATCACAAC | ggtgtgttga | GTAATCACAAC | | |
| | | | (SEQ ID. NO: 1677) | (SEQ ID. | (SEQ ID. | | |
| | | | | NO: | NO: 1679) | | |
| | | | | 1678) | | | |
| 7a | dengue_5 | CcaCas13b | gagagctggtataatcccttctggt | gagagctgg | GTTGGAACTGCT | Dengue | 7a |
| | 1 | | gtgttGTTGGAACTGCTCTCATTTT | tataatccctt | CTCATTTTGGAGG | ssRNA | |
| | | | GGAGGGTAATCACAAC | ctggtgtgtt | GTAATCACAAC | | |
| | | | (SEQ ID. NO: 1680) | (SEQ ID. | (SEQ ID. | | |
| | | | | NO: | NO: 1682) | | |
| | | | | 1681) | | | |
| 7a | dengue_5 | CcaCas13b | aagagagctggtataatcccttctg | aagagagct | GTTGGAACTGCT | Dengue | 7a |
| | 2 | | gtgtgGTTGGAACTGCTCTCATTTT | ggtataatcc | CTCATTTTGGAGG | ssRNA | |
| | | | GGAGGGTAATCACAAC | cttctggtgt | GTAATCACAAC | | |
| | | | (SEQ ID. NO: 1683) | g (SEQ | (SEQ ID. | | |
| | | | | ID. NO: | NO: 1685) | | |
| | | | | 1684) | | | |
| 7a | dengue_5 | CcaCas13b | caaagagagctggtataatcccttc | caaagagag | GTTGGAACTGCT | Dengue | 7a |
| | 3 | | tggtgGTTGGAACTGCTCTCATTTT | ctggtataat | CTCATTTTGGAGG | ssRNA | |
| | | | GGAGGGTAATCACAAC | cccttctggt | GTAATCACAAC | | |
| | | | (SEQ ID. NO: 1686) | g (SEQ | (SEQ ID. | | |
| | | | | ID. NO: | NO: 1688) | | |
| | | | | 1687) | | | |
| 7a | dengue_5 | CcaCas13b | ttcaaagagagctggtataatccct | ttcaaagaga | GTTGGAACTGCT | Dengue | 7a |
| | 4 | | tctggGTTGGAACTGCTCTCATTTT | gctggtataa | CTCATTTTGGAGG | ssRNA | |
| | | | GGAGGGTAATCACAAC | tcccttctgg | GTAATCACAAC | | |
| | | | (SEQ ID. NO: 1689) | (SEQ ID. | (SEQ ID. | | |
| | | | | NO: | NO: 1691) | | |
| | | | | 1690) | | | |
| 7a | dengue_5 | CcaCas13b | ggttcaaagagagctggtataatcc | ggttcaaag | GTTGGAACTGCT | Dengue | 7a |
| | 5 | | cttctGTTGGAACTGCTCTCATTTT | agagctggt | CTCATTTTGGAGG | ssRNA | |
| | | | GGAGGGTAATCACAAC | ataatcccttc | GTAATCACAAC | | |
| | | | (SEQ ID. NO: 1692) | t (SEQ | (SEQ ID. | | |
| | | | | ID. NO: | NO: 1694) | | |
| | | | | 1693) | | | |
| 7a | dengue_5 | CcaCas13b | ctggttcaaagagagctggtataat | ctggttcaaa | GTTGGAACTGCT | Dengue | 7a |
| | 6 | | cccttGTTGGAACTGCTCTCATTTT | gagagctgg | CTCATTTTGGAGG | ssRNA | |
| | | | GGAGGGTAATCACAAC | tataatccctt | GTAATCACAAC | | |
| | | | (SEQ ID. NO: 1695) | (SEQ ID. | (SEQ ID. | | |
| | | | | NO: | NO: 1697) | | |
| | | | | 1696) | | | |
| 7a | dengue_5 | CcaCas13b | ttctggttcaaagagagctggtata | ttctggttcaa | GTTGGAACTGCT | Dengue | 7a |
| | 7 | | atcccGTTGGAACTGCTCTCATTTT | agagagctg | CTCATTTTGGAGG | ssRNA | |
| | | | GGAGGGTAATCACAAC | gtataatccc | GTAATCACAAC | | |
| | | | (SEQ ID. NO: 1698) | (SEQ ID. | (SEQ ID. | | |
| | | | | NO: | NO: 1700) | | |
| | | | | 1699) | | | |
| 7a | dengue_5 | CcaCas13b | ctttctggttcaaagagagctggta | ctttctggttc | GTTGGAACTGCT | Dengue | 7a |
| | 8 | | taatcGTTGGAACTGCTCTCATTTT | aaagagagc | CTCATTTTGGAGG | ssRNA | |
| | | | GGAGGGTAATCACAAC | tggtataatc | GTAATCACAAC | | |
| | | | (SEQ ID. NO: 1701) | (SEQ ID. | (SEQ ID. | | |
| | | | | NO: | NO: 1703) | | |
| | | | | 1702) | | | |
| 7a | dengue_5 | CcaCas13b | ccctttctggttcaaagagagctgg | ccctttctggt | GTTGGAACTGCT | Dengue | 7a |
| | 9 | | tataaGTTGGAACTGCTCTCATTTT | tcaaagaga | CTCATTTTGGAGG | ssRNA | |
| | | | GGAGGGTAATCACAAC | gctggtataa | GTAATCACAAC | | |
| | | | (SEQ ID. NO: 1704) | (SEQ ID. | (SEQ ID. | | |
| | | | | NO: | NO: 1706) | | |
| | | | | 1705) | | | |
| 7a | dengue_6 | CcaCas13b | ctccctttctggttcaaagagagct | ctccctttctg | GTTGGAACTGCT | Dengue | 7a |
| | 0 | | ggtatGTTGGAACTGCTCTCATTTT | gttcaaaga | CTCATTTTGGAGG | ssRNA | |
| | | | GGAGGGTAATCACAAC | gagctggtat | GTAATCACAAC | | |
| | | | (SEQ ID. NO: 1707) | (SEQ ID. | (SEQ ID. | | |
| | | | | NO: | NO: 1709) | | |
| | | | | 1708) | | | |
| 7a | dengue_6 | CcaCas13b | ttctccctttctggttcaaagagag | ttctccctttc | GTTGGAACTGCT | Dengue | 7a |
| | 1 | | ctggtGTTGGAACTGCTCTCATTTT | tggttcaaag | CTCATTTTGGAGG | ssRNA | |
| | | | GGAGGGTAATCACAAC | agagctggt | GTAATCACAAC | | |
| | | | (SEQ ID. NO: 1710) | (SEQ ID. | (SEQ ID. | | |
| | | | | NO: | NO: 1712) | | |
| | | | | 1711) | | | |
| 7a | dengue_6 | CcaCas13b | acttctccctttctggttcaaagag | acttctccctt | GTTGGAACTGCT | Dengue | 7a |
| | 2 | | agctgGTTGGAACTGCTCTCATTTT | tctggttcaa | CTCATTTTGGAGG | ssRNA | |
| | | | GGAGGGTAATCACAAC | agagagctg | GTAATCACAAC | | |
| | | | (SEQ ID. NO: 1713) | (SEQ ID. | (SEQ ID. | | |
| | | | | NO: | NO: 1715) | | |
| | | | | 1714) | | | |
| 7a | dengue_6 | CcaCas13b | tgacttctccctttctggttcaaag | tgacttctcc | GTTGGAACTGCT | Dengue | 7a |
| | 3 | | agagcGTTGGAACTGCTCTCATTTT | ctttctggttc | CTCATTTTGGAGG | ssRNA | |
| | | | GGAGGGTAATCACAAC | aaagagagc | GTAATCACAAC | | |
| | | | (SEQ ID. NO: 1716) | (SEQ ID. | (SEQ ID. | | |
| | | | | NO: | NO: 1718) | | |
| | | | | 1717) | | | |
| 7a | dengue_6 | CcaCas13b | gctgacttctccctttctggttcaa | gctgacttct | GTTGGAACTGCT | Dengue | 7a |
| | 4 | | agagaGTTGGAACTGCTCTCATTTT | ccctttctggt | CTCATTTTGGAGG | ssRNA | |
| | | | GGAGGGTAATCACAAC | tcaaagaga | GTAATCACAAC | | |
| | | | (SEQ ID. NO: 1719) | (SEQ ID. | (SEQ ID. | | |
| | | | | NO: | NO: 1721) | | |
| | | | | 1720) | | | |
| 7a | dengue_6 | CcaCas13b | ggctgacttctccctttctggttca | ggctgacttc | GTTGGAACTGCT | Dengue | 7a |
| | 5 | | aagagGTTGGAACTGCTCTCATTTT | tccctttctgg | CTCATTTTGGAGG | ssRNA | |
| | | | GGAGGGTAATCACAAC | ttcaaagag | GTAATCACAAC | | |
| | | | (SEQ ID. NO: 1722) | (SEQ ID. | (SEQ ID. | | |
| | | | | NO: | NO: 1724) | | |
| | | | | 1723) | | | |
| 7a | dengue_6 | CcaCas13b | cggctgacttctccctttctggttc | cggctgactt | GTTGGAACTGCT | Dengue | 7a |
| | 6 | | aaagaGTTGGAACTGCTCTCATTTT | ctccctttctg | CTCATTTTGGAGG | ssRNA | |
| | | | GGAGGGTAATCACAAC | gttcaaaga | GTAATCACAAC | | |
| | | | (SEQ ID. NO: 1725) | (SEQ ID. | (SEQ ID. | | |
| | | | | NO: | NO: 1727) | | |
| | | | | 1726) | | | |
| 7a | dengue_6 | CcaCas13b | gcggctgacttctccctttctggtt | gcggctgac | GTTGGAACTGCT | Dengue | 7a |
| | 7 | | caaagGTTGGAACTGCTCTCATTTT | ttctccctttc | CTCATTTTGGAGG | ssRNA | |
| | | | GGAGGGTAATCACAAC | tggttcaaag | GTAATCACAAC | | |
| | | | (SEQ ID. NO: 1728) | (SEQ ID. | (SEQ ID. | | |
| | | | | NO: | NO: 1730) | | |
| | | | | 1729) | | | |
| 7a | dengue_6 | CcaCas13b | ggcggctgacttctccctttctggt | ggcggctga | GTTGGAACTGCT | Dengue | 7a |
| | 8 | | tcaaaGTTGGAACTGCTCTCATTTT | cttctcccttt | CTCATTTTGGAGG | ssRNA | |
| | | | GGAGGGTAATCACAAC | ctggttcaaa | GTAATCACAAC | | |
| | | | (SEQ ID. NO: 1731) | (SEQ ID. | (SEQ ID. | | |
| | | | | NO: | NO: 1733) | | |
| | | | | 1732) | | | |
| 7a | dengue_6 | CcaCas13b | tggcggctgacttctccctttctgg | tggcggctg | GTTGGAACTGCT | Dengue | 7a |
| | 9 | | ttcaaGTTGGAACTGCTCTCATTTT | acttctccctt | CTCATTTTGGAGG | ssRNA | |
| | | | GGAGGGTAATCACAAC | tctggttcaa | GTAATCACAAC | | |
| | | | (SEQ ID. NO: 1734) | (SEQ ID. | (SEQ ID. | | |
| | | | | NO: | NO: 1736) | | |
| | | | | 1735) | | | |
| 7a | dengue_7 | CcaCas13b | atggcggctgacttctccctttctg | atggcggct | GTTGGAACTGCT | Dengue | 7a |
| | 0 | | gttcaGTTGGAACTGCTCTCATTTT | gacttctccc | CTCATTTTGGAGG | ssRNA | |
| | | | GGAGGGTAATCACAAC | tttctggttca | GTAATCACAAC | | |
| | | | (SEQ ID. NO: 1737) | (SEQ ID. | (SEQ ID. | | |
| | | | | NO: | NO: 1739) | | |
| | | | | 1738) | | | |
| 7a | dengue_7 | CcaCas13b | tatggcggctgacttctccctttct | tatggcggct | GTTGGAACTGCT | Dengue | 7a |
| | 1 | | ggttcGTTGGAACTGCTCTCATTTT | gacttctccc | CTCATTTTGGAGG | ssRNA | |
| | | | GGAGGGTAATCACAAC | tttctggttc | GTAATCACAAC | | |
| | | | (SEQ ID. NO: 1740) | (SEQ ID. | (SEQ ID. | | |
| | | | | NO: | NO: 1742) | | |
| | | | | 1741) | | | |
| 7a | dengue_7 | CcaCas13b | ctatggcggctgacttctccctttc | ctatggcgg | GTTGGAACTGCT | Dengue | 7a |
| | 2 | | tggttGTTGGAACTGCTCTCATTTT | ctgacttctc | CTCATTTTGGAGG | ssRNA | |
| | | | GGAGGGTAATCACAAC | cctttctggtt | GTAATCACAAC | | |
| | | | (SEQ ID. NO: 1743) | (SEQ ID. | (SEQ ID. | | |
| | | | | NO: | NO: 1745) | | |
| | | | | 1744) | | | |
| 7a | dengue_7 | CcaCas13b | tctatggcggctgacttctcccttt | tctatggcgg | GTTGGAACTGCT | Dengue | 7a |
| | 3 | | ctggtGTTGGAACTGCTCTCATTTT | ctgacttctc | CTCATTTTGGAGG | ssRNA | |
| | | | GGAGGGTAATCACAAC | cctttctggt | GTAATCACAAC | | |
| | | | (SEQ ID. NO: 1746) | (SEQ ID. | (SEQ ID. | | |
| | | | | NO: | NO: 1748) | | |
| | | | | 1747) | | | |
| 7a | dengue_7 | CcaCas13b | gtctatggcggctgacttctccctt | gtctatggcg | GTTGGAACTGCT | Dengue | 7a |
| | 4 | | tctggGTTGGAACTGCTCTCATTTT | gctgacttct | CTCATTTTGGAGG | ssRNA | |
| | | | GGAGGGTAATCACAAC | ccctttctgg | GTAATCACAAC | | |
| | | | (SEQ ID. NO: 1749) | (SEQ ID. | (SEQ ID. | | |
| | | | | NO: | NO: 1751) | | |
| | | | | 1750) | | | |
| 7a | dengue_7 | CcaCas13b | cgtctatggcggctgacttctccct | cgtctatggc | GTTGGAACTGCT | Dengue | 7a |
| | 5 | | ttctgGTTGGAACTGCTCTCATTTT | ggctgacttc | CTCATTTTGGAGG | ssRNA | |
| | | | GGAGGGTAATCACAAC | tccctttctg | GTAATCACAAC | | |
| | | | (SEQ ID. NO: 1752) | (SEQ ID. | (SEQ ID. | | |
| | | | | NO: | NO: 1754) | | |
| | | | | 1753) | | | |
| 7a | dengue_7 | CcaCas13b | ccgtctatggcggctgacttctccc | ccgtctatgg | GTTGGAACTGCT | Dengue | 7a |
| | 6 | | tttctGTTGGAACTGCTCTCATTTT | cggctgactt | CTCATTTTGGAGG | ssRNA | |
| | | | GGAGGGTAATCACAAC | ctccctttct | GTAATCACAAC | | |
| | | | (SEQ ID. NO: 1755) | (SEQ ID. | (SEQ ID. | | |
| | | | | NO: | NO: 1757) | | |
| | | | | 1756) | | | |
| 7a | dengue_7 | CcaCas13b | accgtctatggcggctgacttctcc | accgtctatg | GTTGGAACTGCT | Dengue | 7a |
| | 7 | | ctttcGTTGGAACTGCTCTCATTTT | gcggctgac | CTCATTTTGGAGG | ssRNA | |
| | | | GGAGGGTAATCACAAC | ttctccctttc | GTAATCACAAC | | |
| | | | (SEQ ID. NO: 1758) | (SEQ ID. | (SEQ ID. | | |
| | | | | NO: | NO: 1760) | | |
| | | | | 1759) | | | |
| 7a | dengue_7 | CcaCas3b | caccgtctatggcggctgacttctc | caccgtctat | GTTGGAACTGCT | Dengue | 7a |
| | 8 | | cctttGTTGGAACTGCTCTCATTTT | ggcggctga | CTCATTTTGGAGG | ssRNA | |
| | | | GGAGGGTAATCACAAC | cttctcccttt | GTAATCACAAC | | |
| | | | (SEQ ID. NO: 1761) | (SEQ ID. | (SEQ ID. | | |
| | | | | NO: | NO: 1763) | | |
| | | | | 1762) | | | |
| 7a | dengue_7 | CcaCas13b | tcaccgtctatggcggctgacttct | tcaccgtcta | GTTGGAACTGCT | Dengue | 7a |
| | 9 | | cccttGTTGGAACTGCTCTCATTTT | tggcggctg | CTCATTTTGGAGG | ssRNA | |
| | | | GGAGGGTAATCACAAC | acttctccctt | GTAATCACAAC | | |
| | | | (SEQ ID. NO: 1764) | (SEQ ID. | (SEQ ID. | | |
| | | | | NO: | NO: 1766) | | |
| | | | | 1765) | | | |
| 7a | dengue_8 | CcaCas13b | ttcaccgtctatggcggctgacttc | ttcaccgtct | GTTGGAACTGCT | Dengue | 7a |
| | 0 | | tccctGTTGGAACTGCTCTCATTTT | atggcggct | CTCATTTTGGAGG | ssRNA | |
| | | | GGAGGGTAATCACAAC | gacttctccc | GTAATCACAAC | | |
| | | | (SEQ ID. NO: 1767) | t (SEQ | (SEQ ID. | | |
| | | | | ID. NO: | NO: 1769) | | |
| | | | | 1768) | | | |
| 7a | dengue_8 | CcaCas13b | attcaccgtctatggcggctgactt | attcaccgtc | GTTGGAACTGCT | Dengue | 7a |
| | 1 | | ctcccGTTGGAACTGCTCTCATTTT | tatggcggct | CTCATTTTGGAGG | ssRNA | |
| | | | GGAGGGTAATCACAAC | gacttctccc | GTAATCACAAC | | |
| | | | (SEQ ID. NO: 1770) | (SEQ ID. | (SEQ ID. | | |
| | | | | NO: | NO: 1772) | | |
| | | | | 1771) | | | |
| 7a | dengue_8 | CcaCas13b | tattcaccgtctatggcggctgact | tattcaccgt | GTTGGAACTGCT | Dengue | 7a |
| | 2 | | tctccGTTGGAACTGCTCTCATTTT | ctatggcgg | CTCATTTTGGAGG | ssRNA | |
| | | | GGAGGGTAATCACAAC | ctgacttctc | GTAATCACAAC | | |
| | | | (SEQ ID. NO: 1773) | c (SEQ | (SEQ ID. | | |
| | | | | ID. NO: | NO: 1775) | | |
| | | | | 1774) | | | |
| 7a | dengue_8 | CcaCas13b | gtattcaccgtctatggcggctgac | gtattcaccg | GTTGGAACTGCT | Dengue | 7a |
| | 3 | | ttctcGTTGGAACTGCTCTCATTTT | tctatggcgg | CTCATTTTGGAGG | ssRNA | |
| | | | GGAGGGTAATCACAAC | ctgacttctc | GTAATCACAAC | | |
| | | | (SEQ ID. NO: 1776) | (SEQ ID. | (SEQ ID. | | |
| | | | | NO: | NO: 1778) | | |
| | | | | 1777) | | | |
| 7a | dengue_8 | CcaCas13b | ggtattcaccgtctatggcggctga | ggtattcacc | GTTGGAACTGCT | Dengue | 7a |
| | 4 | | cttctGTTGGAACTGCTCTCATTTT | gtctatggcg | CTCATTTTGGAGG | ssRNA | |
| | | | GGAGGGTAATCACAAC | gctgacttct | GTAATCACAAC | | |
| | | | (SEQ ID. NO: 1779) | (SEQ ID. | (SEQ ID. | | |
| | | | | NO: | NO: 1781) | | |
| | | | | 1780) | | | |
| 7a | dengue_8 | CcaCas13b | cggtattcaccgtctatggcggctg | cggtattcac | GTTGGAACTGCT | Dengue | 7a |
| | 5 | | acttcGTTGGAACTGCTCTCATTTT | cgtctatggc | CTCATTTTGGAGG | ssRNA | |
| | | | GGAGGGTAATCACAAC | ggctgacttc | GTAATCACAAC | | |
| | | | (SEQ ID. NO: 1782) | (SEQ ID. | (SEQ ID. | | |
| | | | | NO: | NO: 1784) | | |
| | | | | 1783) | | | |
| 7a | dengue_8 | CcaCas13b | gcggtattcaccgtctatggcggct | gcggtattca | GTTGGAACTGCT | Dengue | 7a |
| | 6 | | gacttGTTGGAACTGCTCTCATTTT | ccgtctatgg | CTCATTTTGGAGG | ssRNA | |
| | | | GGAGGGTAATCACAAC | cggctgactt | GTAATCACAAC | | |
| | | | (SEQ ID. NO: 1785) | (SEQ ID. | (SEQ ID. | | |
| | | | | NO: | NO: 1787) | | |
| | | | | 1786) | | | |
| 7a | dengue_8 | CcaCas13b | ggcggtattcaccgtctatggcggc | ggcggtattc | GTTGGAACTGCT | Dengue | 7a |
| | 7 | | tgactGTTGGAACTGCTCTCATTT | accgtctatg | CTCATTTTGGAGG | ssRNA | |
| | | | TGGAGGGTAATCACAAC | gcggctgac | GTAATCACAAC | | |
| | | | (SEQ ID. NO: 1788) | t (SEQ | (SEQ ID. | | |
| | | | | ID. NO: | NO: 1790) | | |
| | | | | 1789) | | | |
| 7a | dengue_8 | CcaCas13b | aggcggtattcaccgtctatggcgg | aggcggtatt | GTTGGAACTGCT | Dengue | 7a |
| | 8 | | ctgacGTTGGAACTGCTCTCATTT | caccgtctat | CTCATTTTGGAGG | ssRNA | |
| | | | TGGAGGGTAATCACAAC | ggcggctga | GTAATCACAAC | | |
| | | | (SEQ ID. NO: 1791) | c (SEQ | (SEQ ID. | | |
| | | | | ID. NO: | NO: 1793) | | |
| | | | | 1792) | | | |
| 7a | dengue_8 | CcaCas13b | caggcggtattcaccgtctatggcg | caggcggta | GTTGGAACTGCT | Dengue | 7a |
| | 9 | | gctgaGTTGGAACTGCTCTCATTTT | ttcaccgtct | CTCATTTTGGAGG | ssRNA | |
| | | | GGAGGGTAATCACAAC | atggcggct | GTAATCACAAC | | |
| | | | (SEQ ID. NO: 1794) | ga (SEQ | (SEQ ID. | | |
| | | | | ID. NO: | NO: 1796) | | |
| | | | | 1795) | | | |
| 7a | dengue_9 | CcaCas13b | tcaggcggtattcaccgtctatggc | tcaggcggt | GTTGGAACTGCT | Dengue | 7a |
| | 0 | | ggctgGTTGGAACTGCTCTCATTTT | attcaccgtc | CTCATTTTGGAGG | ssRNA | |
| | | | GGAGGGTAATCACAAC | tatggcggct | GTAATCACAAC | | |
| | | | (SEQ ID. NO: 1797) | g (SEQ | (SEQ ID. | | |
| | | | | ID. NO: | NO: 1799) | | |
| | | | | 1798) | | | |
| 7a | dengue_9 | CcaCas13b | ttcaggcggtattcaccgtctatgg | ttcaggcggt | GTTGGAACTGCT | Dengue | 7a |
| | 1 | | cggctGTTGGAACTGCTCTCATTTT | attcaccgtc | CTCATTTTGGAGG | ssRNA | |
| | | | GGAGGGTAATCACAAC | tatggcggct | GTAATCACAAC | | |
| | | | (SEQ ID. NO: 1800) | (SEQ ID. | (SEQ ID. | | |
| | | | | NO: | NO: 1802) | | |
| | | | | 1801) | | | |
| 7a | dengue_9 | CcaCas13b | cttcaggcggtattcaccgtctatg | cttcaggcg | GTTGGAACTGCT | Dengue | 7a |
| | 2 | | gcggcGTTGGAACTGCTCTCATTTT | gtattcaccg | CTCATTTTGGAGG | ssRNA | |
| | | | GGAGGGTAATCACAAC | tctatggcgg | GTAATCACAAC | | |
| | | | (SEQ ID. NO: 1803) | c (SEQ | (SEQ ID. | | |
| | | | | ID. NO: | NO: 1805) | | |
| | | | | 1804) | | | |
| 7a | dengue_9 | CcaCas13b | ccttcaggcggtattcaccgtctat | ccttcaggc | GTTGGAACTGCT | Dengue | 7a |
| | 3 | | ggcggGTTGGAACTGCTCTCATTTT | ggtattcacc | CTCATTTTGGAGG | ssRNA | |
| | | | GGAGGGTAATCACAAC | gtctatggcg | GTAATCACAAC | | |
| | | | (SEQ ID. NO: 1806) | g (SEQ | (SEQ ID. | | |
| | | | | ID. NO: | NO: 1808) | | |
| | | | | 1807) | | | |
| 7a | dengue_9 | CcaCas13b | cccttcaggcggtattcaccgtcta | cccttcaggc | GTTGGAACTGCT | Dengue | 7a |
| | 4 | | tggcgGTTGGAACTGCTCTCATTTT | ggtattcacc | CTCATTTTGGAGG | ssRNA | |
| | | | GGAGGGTAATCACAAC | gtctatggcg | GTAATCACAAC | | |
| | | | (SEQ ID. NO: 1809) | (SEQ ID. | (SEQ ID. | | |
| | | | | NO: | NO: 1811) | | |
| | | | | 1810) | | | |
| 7a | thermo_0 | CcaCas13b | attaatttaacagtatcaccatcaa | attaatttaac | GTTGGAACTGCT | Ther- | 7a |
| | | | tcgctGTTGGAACTGCTCTCATTTT | agtatcacca | CTCATTTTGGAGG | mo- | |
| | | | GGAGGGTAATCACAAC | tcaatcgct | GTAATCACAAC | nu- | |
| | | | (SEQ ID. NO: 1812) | (SEQ ID. | (SEQ ID. | clease | |
| | | | | NO: | NO: 1814) | | |
| | | | | 1813) | | | |
| 7a | thermo_1 | CcaCas13b | cattaatttaacagtatcaccatca | cattaatttaa | GTTGGAACTGCT | Ther- | 7a |
| | | | atcgcGTTGGAACTGCTCTCATTTT | cagtatcacc | CTCATTTTGGAGG | mo- | |
| | | | GGAGGGTAATCACAAC | atcaatcgc | GTAATCACAAC | nu- | |
| | | | (SEQ ID. NO: 1815) | (SEQ ID. | (SEQ ID. | clease | |
| | | | | NO: | NO: 1817) | | |
| | | | | 1816) | | | |
| 7a | thermo_2 | CcaCas13b | acattaatttaacagtatcaccatc | acattaattta | GTTGGAACTGCT | Ther- | 7a |
| | | | aatcgGTTGGAACTGCTCTCATTTT | acagtatcac | CTCATTTTGGAGG | mo- | |
| | | | GGAGGGTAATCACAAC | catcaatcg | GTAATCACAAC | nu- | |
| | | | (SEQ ID. NO: 1818) | (SEQ ID. | (SEQ ID. | clease | |
| | | | | NO: | NO: 1820) | | |
| | | | | 1819) | | | |
| 7a | thermo_3 | CcaCas13b | tacattaatttaacagtatcaccat | tacattaattt | GTTGGAACTGCT | Ther- | 7a |
| | | | caatcGTTGGAACTGCTCTCATTTT | aacagtatca | CTCATTTTGGAGG | mo- | |
| | | | GGAGGGTAATCACAAC | ccatcaatc | GTAATCACAAC | nu- | |
| | | | (SEQ ID. NO: 1821) | (SEQ ID. | (SEQ ID. | clease | |
| | | | | NO: | NO: 1823) | | |
| | | | | 1822) | | | |
| 7a | thermo_4 | CcaCas13b | gtacattaatttaacagtatcacca | gtacattaatt | GTTGGAACTGCT | Ther- | 7a |
| | | | tcaatGTTGGAACTGCTCTCATTTT | taacagtatc | CTCATTTTGGAGG | mo- | |
| | | | GGAGGGTAATCACAAC | accatcaat | GTAATCACAAC | nu- | |
| | | | (SEQ ID. NO: 1824) | (SEQ ID. | (SEQ ID. | clease | |
| | | | | NO: | NO: 1826) | | |
| | | | | 1825) | | | |
| 7a | thermo_5 | CcaCas13b | tgtacattaatttaacagtatcacc | tgtacattaat | GTTGGAACTGCT | Ther- | 7a |
| | | | atcaaGTTGGAACTGCTCTCATTTT | ttaacagtat | CTCATTTTGGAGG | mo- | |
| | | | GGAGGGTAATCACAAC | caccatcaa | GTAATCACAAC | nu- | |
| | | | (SEQ ID. NO: 1827) | (SEQ ID. | (SEQ ID. | clease | |
| | | | | NO: | NO: 1829) | | |
| | | | | 1828) | | | |
| 7a | thermo_6 | CcaCas13b | ttgtacattaatttaacagtatcac | ttgtacattaa | GTTGGAACTGCT | Ther- | 7a |
| | | | catcaGTTGGAACTGCTCTCATTTT | tttaacagtat | CTCATTTTGGAGG | mo- | |
| | | | GGAGGGTAATCACAAC | caccatca | GTAATCACAAC | nu- | |
| | | | (SEQ ID. NO: 1830) | (SEQ ID. | (SEQ ID. | clease | |
| | | | | NO: | NO: 1832) | | |
| | | | | 1831) | | | |
| 7a | thermo_7 | CcaCas13b | tttgtacattaatttaacagtatca | tttgtacatta | GTTGGAACTGCT | Ther- | 7a |
| | | | ccatcGTTGGAACTGCTCTCATTTT | atttaacagt | CTCATTTTGGAGG | mo- | |
| | | | GGAGGGTAATCACAAC | atcaccatc | GTAATCACAAC | nu- | |
| | | | (SEQ ID. NO: 1833) | (SEQ ID. | (SEQ ID. | clease | |
| | | | | NO: | NO: 1835) | | |
| | | | | 1834) | | | |
| 7a | thermo_8 | CcaCas13b | ctttgtacattaatttaacagtatc | ctttgtacatt | GTTGGAACTGCT | Ther- | 7a |
| | | | accatGTTGGAACTGCTCTCATTTT | aatttaacag | CTCATTTTGGAGG | mo- | |
| | | | GGAGGGTAATCACAAC | tatcaccat | GTAATCACAAC | nu- | |
| | | | (SEQ ID. NO: 1836) | (SEQ ID. | (SEQ ID. | clease | |
| | | | | NO: | NO: 1838) | | |
| | | | | 1837) | | | |
| 7a | thermo_9 | CcaCas13b | cctttgtacattaatttaacagtat | cctttgtacat | GTTGGAACTGCT | Ther- | 7a |
| | | | caccaGTTGGAACTGCTCTCATTTT | taatttaaca | CTCATTTTGGAGG | mo- | |
| | | | GGAGGGTAATCACAAC | gtatcacca | GTAATCACAAC | nu- | |
| | | | (SEQ ID. NO: 1839) | (SEQ ID. | (SEQ ID. | clease | |
| | | | | NO: | NO: 1841) | | |
| | | | | 1840) | | | |
| 7a | thermo_1 | CcaCas13b | acctttgtacattaatttaacagta | acctttgtac | GTTGGAACTGCT | Ther- | 7a |
| | 0 | | tcaccGTTGGAACTGCTCTCATTTT | attaatttaac | CTCATTTTGGAGG | mo- | |
| | | | GGAGGGTAATCACAAC | agtatcacc | GTAATCACAAC | nu- | |
| | | | (SEQ ID. NO: 1842) | (SEQ ID. | (SEQ ID. | clease | |
| | | | | NO: | NO: 1844) | | |
| | | | | 1843) | | | |
| 7a | thermo_1 | CcaCas13b | gacctttgtacattaatttaacagt | gacctttgta | GTTGGAACTGCT | Ther- | 7a |
| | 1 | | atcacGTTGGAACTGCTCTCATTTT | cattaatttaa | CTCATTTTGGAGG | mo- | |
| | | | GGAGGGTAATCACAAC | cagtatcac | GTAATCACAAC | nu- | |
| | | | (SEQ ID. NO: 1845) | (SEQ ID. | (SEQ ID. | clease | |
| | | | | NO: | NO: 1847) | | |
| | | | | 1846) | | | |
| 7a | thermo_1 | CcaCas13b | tgacctttgtacattaatttaacag | tgacctttgta | GTTGGAACTGCT | Ther- | 7a |
| | 2 | | tatcaGTTGGAACTGCTCTCATTTT | cattaatttaa | CTCATTTTGGAGG | mo- | |
| | | | GGAGGGTAATCACAAC | cagtatca | GTAATCACAAC | nu- | |
| | | | (SEQ ID. NO: 1848) | (SEQ ID. | (SEQ ID. | clease | |
| | | | | NO: | NO: 1850) | | |
| | | | | 1849) | | | |
| 7a | thermo_1 | CcaCas13b | ttgacctttgtacattaatttaaca | ttgacctttgt | GTTGGAACTGCT | Ther- | 7a |
| | 3 | | gtatcGTTGGAACTGCTCTCATTTT | acattaattta | CTCATTTTGGAGG | mo- | |
| | | | GGAGGGTAATCACAAC | acagtatc | GTAATCACAAC | nu- | |
| | | | (SEQ ID. NO: 1851) | (SEQ ID. | (SEQ ID. | clease | |
| | | | | NO: | NO: 1853) | | |
| | | | | 1852) | | | |
| 7a | thermo_1 | CcaCas13b | gttgacctttgtacattaatttaac | gttgacctttg | GTTGGAACTGCT | Ther- | 7a |
| | 4 | | agtatGTTGGAACTGCTCTCATTTT | tacattaattt | CTCATTTTGGAGG | mo- | |
| | | | GGAGGGTAATCACAAC | aacagtat | GTAATCACAAC | nu- | |
| | | | (SEQ ID. NO: 1854) | (SEQ ID. | (SEQ ID. | clease | |
| | | | | NO: | NO: 1856) | | |
| | | | | 1855) | | | |
| 7a | thermo_1 | CcaCas13b | ggttgacctttgtacattaatttaa | ggttgaccttt | GTTGGAACTGCT | Ther- | 7a |
| | 5 | | cagtaGTTGGAACTGCTCTCATTTT | gtacattaatt | CTCATTTTGGAGG | mo- | |
| | | | GGAGGGTAATCACAAC | taacagta | GTAATCACAAC | nu- | |
| | | | (SEQ ID. NO: 1857) | (SEQ ID. | (SEQ ID. | clease | |
| | | | | NO: | NO: 1859) | | |
| | | | | 1858) | | | |
| 7a | thermo_1 | CcaCas13b | tggttgacctttgtacattaattta | tggttgacctt | GTTGGAACTGCT | Ther- | 7a |
| | 6 | | acagtGTTGGAACTGCTCTCATTTT | tgtacattaat | CTCATTTTGGAGG | mo- | |
| | | | GGAGGGTAATCACAAC | ttaacagt | GTAATCACAAC | nu- | |
| | | | (SEQ ID. NO: 1860) | (SEQ ID. | (SEQ ID. | clease | |
| | | | | NO: | NO: 1862) | | |
| | | | | 1861) | | | |
| 7a | thermo_1 | CcaCas13b | ttggttgacctttgtacattaattt | ttggttgacct | GTTGGAACTGCT | Ther- | 7a |
| | 7 | | aacagGTTGGAACTGCTCTCATTTT | ttgtacattaa | CTCATTTTGGAGG | mo- | |
| | | | GGAGGGTAATCACAAC | tttaacag | GTAATCACAAC | nu- | |
| | | | (SEQ ID. NO: 1863) | (SEQ ID. | (SEQ ID. | clease | |
| | | | | NO: | NO: 1865) | | |
| | | | | 1864) | | | |
| 7a | thermo_1 | CcaCas13b | attggttgacctttgtacattaatt | attggttgac | GTTGGAACTGCT | Ther- | 7a |
| | 8 | | taacaGTTGGAACTGCTCTCATTTT | ctttgtacatt | CTCATTTTGGAGG | mo- | |
| | | | GGAGGGTAATCACAAC | aatttaaca | GTAATCACAAC | nu- | |
| | | | (SEQ ID. NO: 1866) | (SEQ ID. | (SEQ ID. | clease | |
| | | | | NO: | NO: 1868) | | |
| | | | | 1867) | | | |
| 7a | thermo_1 | CcaCas13b | cattggttgacctttgtacattaat | cattggttga | GTTGGAACTGCT | Ther- | 7a |
| | 9 | | ttaacGTTGGAACTGCTCTCATTTT | cctttgtacat | CTCATTTTGGAGG | mo- | |
| | | | GGAGGGTAATCACAAC | taatttaac | GTAATCACAAC | nu- | |
| | | | (SEQ ID. NO: 1869) | (SEQ ID. | (SEQ ID. | clease | |
| | | | | NO: | NO: 1871) | | |
| | | | | 1870) | | | |
| 7a | thermo_2 | CcaCas13b | gtcattggttgacctttgtacatta | gtcattggtt | GTTGGAACTGCT | Ther- | 7a |
| | 0 | | atttaGTTGGAACTGCTCTCATTTT | gacctttgta | CTCATTTTGGAGG | mo- | |
| | | | GGAGGGTAATCACAAC | cattaattta | GTAATCACAAC | nu- | |
| | | | (SEQ ID. NO: 1872) | (SEQ ID. | (SEQ ID. | clease | |
| | | | | NO: | NO: 1874) | | |
| | | | | 1873) | | | |
| 7a | thermo_2 | CcaCas13b | atgtcattggttgacctttgtacat | atgtcattggt | GTTGGAACTGCT | Ther- | 7a |
| | 1 | | taattGTTGGAACTGCTCTCATTTT | tgacctttgta | CTCATTTTGGAGG | mo- | |
| | | | GGAGGGTAATCACAAC | cattaatt | GTAATCACAAC | nu- | |
| | | | (SEQ ID. NO: 1875) | (SEQ ID. | (SEQ ID. | clease | |
| | | | | NO: | NO: 1877) | | |
| | | | | 1876) | | | |
| 7a | thermo_2 | CcaCas13b | gaatgtcattggttgacctttgtac | gaatgtcatt | GTTGGAACTGCT | Ther- | 7a |
| | 2 | | attaaGTTGGAACTGCTCTCATTTT | ggttgaccttt | CTCATTTTGGAGG | mo- | |
| | | | GGAGGGTAATCACAAC | gtacattaa | GTAATCACAAC | nu- | |
| | | | (SEQ ID. NO: 1878) | (SEQ ID. | (SEQ ID. | clease | |
| | | | | NO: | NO: 1880) | | |
| | | | | 1879) | | | |
| 7a | thermo_2 | CcaCas13b | ctgaatgtcattggttgacctttgt | ctgaatgtca | GTTGGAACTGCT | Ther- | 7a |
| | 3 | | acattGTTGGAACTGCTCTCATTTT | ttggttgacct | CTCATTTTGGAGG | mo- | |
| | | | GGAGGGTAATCACAAC | ttgtacatt | GTAATCACAAC | nu- | |
| | | | (SEQ ID. NO: 1881) | (SEQ ID. | (SEQ ID. | clease | |
| | | | | NO: | NO: 1883) | | |
| | | | | 1882) | | | |
| 7a | thermo_2 | CcaCas13b | gtctgaatgtcattggttgaccttt | gtctgaatgt | GTTGGAACTGCT | Ther- | 7a |
| | 4 | | gtacaGTTGGAACTGCTCTCATTTT | cattggttga | CTCATTTTGGAGG | mo- | |
| | | | GGAGGGTAATCACAAC | cctttgtaca | GTAATCACAAC | nu- | |
| | | | (SEQ ID. NO: 1884) | (SEQ ID. | (SEQ ID. | clease | |
| | | | | NO: | NO: 1886) | | |
| | | | | 1885) | | | |
| 7a | thermo_2 | CcaCas13b | tagtctgaatgtcattggttgacct | tagtctgaat | GTTGGAACTGCT | Ther- | 7a |
| | 5 | | ttgtaGTTGGAACTGCTCTCATTTT | gtcattggtt | CTCATTTTGGAGG | mo- | |
| | | | GGAGGGTAATCACAAC | gacctttgta | GTAATCACAAC | nu- | |
| | | | (SEQ ID. NO: 1887) | (SEQ ID. | (SEQ ID. | clease | |
| | | | | NO: | NO: 1889) | | |
| | | | | 1888) | | | |
| 7a | thermo_2 | CcaCas13b | aatagtctgaatgtcattggttgac | aatagtctga | GTTGGAACTGCT | Ther- | 7a |
| | 6 | | ctttgGTTGGAACTGCTCTCATTTT | atgtcattggt | CTCATTTTGGAGG | mo- | |
| | | | GGAGGGTAATCACAAC | tgacctttg | GTAATCACAAC | nu- | |
| | | | (SEQ ID. NO: 1890) | (SEQ ID. | (SEQ ID. | clease | |
| | | | | NO: | NO: 1892) | | |
| | | | | 1891) | | | |
| 7a | thermo_2 | CcaCas13b | ataatagtctgaatgtcattggttg | ataatagtct | GTTGGAACTGCT | Ther- | 7a |
| | 7 | | accttGTTGGAACTGCTCTCATTTT | gaatgtcatt | CTCATTTTGGAGG | mo- | |
| | | | GGAGGGTAATCACAAC | ggttgacctt | GTAATCACAAC | nu- | |
| | | | (SEQ ID. NO: 1893) | (SEQ ID. | (SEQ ID. | clease | |
| | | | | NO: | NO: 1895) | | |
| | | | | 1894) | | | |
| 7a | thermo_2 | CcaCas13b | caataatagtctgaatgtcattggt | caataatagt | GTTGGAACTGCT | Ther- | 7a |
| | 8 | | tgaccGTTGGAACTGCTCTCATTTT | ctgaatgtca | CTCATTTTGGAGG | mo- | |
| | | | GGAGGGTAATCACAAC | ttggttgacc | GTAATCACAAC | nu- | |
| | | | (SEQ ID. NO: 1896) | (SEQ ID. | (SEQ ID. | clease | |
| | | | | NO: | NO: 1898) | | |
| | | | | 1897) | | | |
| 7a | thermo_2 | CcaCas13b | accaataatagtctgaatgtcattg | accaataata | GTTGGAACTGCT | Ther- | 7a |
| | 9 | | gttgaGTTGGAACTGCTCTCATTTT | gtctgaatgt | CTCATTTTGGAGG | mo- | |
| | | | GGAGGGTAATCACAAC | cattggttga | GTAATCACAAC | nu- | |
| | | | (SEQ ID. NO: 1899) | (SEQ ID. | (SEQ ID. | clease | |
| | | | | NO: | NO: 1901) | | |
| | | | | 1900) | | | |
| 7a | thermo_3 | CcaCas13b | caaccaataatagtctgaatgtcat | caaccaata | GTTGGAACTGCT | Ther- | 7a |
| | 0 | | tggttGTTGGAACTGCTCTCATTTT | atagtctgaa | CTCATTTTGGAGG | mo- | |
| | | | GGAGGGTAATCACAAC | tgtcattggtt | GTAATCACAAC | nu- | |
| | | | (SEQ ID. NO: 1902) | (SEQ ID. | (SEQ ID. | clease | |
| | | | | NO: | NO: 1904) | | |
| | | | | 1903) | | | |
| 7a | thermo_3 | CcaCas13b | atcaaccaataatagtctgaatgtc | atcaaccaat | GTTGGAACTGCT | Ther- | 7a |
| | 1 | | attggGTTGGAACTGCTCTCATTTT | aatagtctga | CTCATTTTGGAGG | mo- | |
| | | | GGAGGGTAATCACAAC | atgtcattgg | GTAATCACAAC | nu- | |
| | | | (SEQ ID. NO: 1905) | (SEQ ID. | (SEQ ID. | clease | |
| | | | | NO: | NO: 1907) | | |
| | | | | 1906) | | | |
| 7a | thermo_3 | CcaCas13b | gtatcaaccaataatagtctgaatg | gtatcaacca | GTTGGAACTGCT | Ther- | 7a |
| | 2 | | tcattGTTGGAACTGCTCTCATTTT | ataatagtct | CTCATTTTGGAGG | mo- | |
| | | | GGAGGGTAATCACAAC | gaatgtcatt | GTAATCACAAC | nu- | |
| | | | (SEQ ID. NO: 1908) | (SEQ ID. | (SEQ ID. | clease | |
| | | | | NO: | NO: 1910) | | |
| | | | | 1909) | | | |
| 7a | thermo_3 | CcaCas13b | gtgtatcaaccaataatagtctgaa | gtgtatcaac | GTTGGAACTGCT | Ther- | 7a |
| | 3 | | tgtcaGTTGGAACTGCTCTCATTTT | caataatagt | CTCATTTTGGAGG | mo- | |
| | | | GGAGGGTAATCACAAC | ctgaatgtca | GTAATCACAAC | nu- | |
| | | | (SEQ ID. NO: 1911) | (SEQ ID. | (SEQ ID. | clease | |
| | | | | NO: | NO: 1913) | | |
| | | | | 1912) | | | |
| 7a | thermo_3 | CcaCas13b | aggtgtatcaaccaataatagtctg | aggtgtatca | GTTGGAACTGCT | Ther- | 7a |
| | 4 | | aatgtGTTGGAACTGCTCTCATTTT | accaataata | CTCATTTTGGAGG | mo- | |
| | | | GGAGGGTAATCACAAC | gtctgaatgt | GTAATCACAAC | nu- | |
| | | | (SEQ ID. NO: 1914) | (SEQ ID. | (SEQ ID. | clease | |
| | | | | NO: | NO: 1916) | | |
| | | | | 1915) | | | |
| 7a | thermo_3 | CcaCas13b | tcaggtgtatcaaccaataatagtc | tcaggtgtat | GTTGGAACTGCT | Ther- | 7a |
| | 5 | | tgaatGTTGGAACTGCTCTCATTTT | caaccaata | CTCATTTTGGAGG | mo- | |
| | | | GGAGGGTAATCACAAC | atagtctgaa | GTAATCACAAC | nu- | |
| | | | (SEQ ID. NO: 1917) | t (SEQ | (SEQ ID. | clease | |
| | | | | ID. NO: | NO: 1919) | | |
| | | | | 1918) | | | |
| 7a | thermo_3 | CcaCas13b | tttcaggtgtatcaaccaataatag | tttcaggtgta | GTTGGAACTGCT | Ther- | 7a |
| | 6 | | tctgaGTTGGAACTGCTCTCATTTT | tcaaccaata | CTCATTTTGGAGG | mo- | |
| | | | GGAGGGTAATCACAAC | atagtctga | GTAATCACAAC | nu- | |
| | | | (SEQ ID. NO: 1920) | (SEQ ID. | (SEQ ID. | clease | |
| | | | | NO: | NO: 1922) | | |
| | | | | 1921) | | | |
| 7a | thermo_3 | CcaCas13b | tgtttcaggtgtatcaaccaataat | tgtttcaggt | GTTGGAACTGCT | Ther- | 7a |
| | 7 | | agtctGTTGGAACTGCTCTCATTTT | gtatcaacca | CTCATTTTGGAGG | mo- | |
| | | | GGAGGGTAATCACAAC | ataatagtct | GTAATCACAAC | nu- | |
| | | | (SEQ ID. NO: 1923) | (SEQ ID. | (SEQ ID. | clease | |
| | | | | NO: | NO: 1925) | | |
| | | | | 1924) | | | |
| 7a | thermo_3 | CcaCas13b | tttgtttcaggtgtatcaaccaata | tttgtttcagg | GTTGGAACTGCT | Ther- | 7a |
| | 8 | | atagtGTTGGAACTGCTCTCATTTT | tgtatcaacc | CTCATTTTGGAGG | mo- | |
| | | | GGAGGGTAATCACAAC | aataatagt | GTAATCACAAC | nu- | |
| | | | (SEQ ID. NO: 1926) | (SEQ ID. | (SEQ ID. | clease | |
| | | | | NO: | NO: 1928) | | |
| | | | | 1927) | | | |
| 7a | thermo_3 | CcaCas13b | gctttgtttcaggtgtatcaaccaa | gctttgtttca | GTTGGAACTGCT | Ther- | 7a |
| | 9 | | taataGTTGGAACTGCTCTCATTTT | ggtgtatcaa | CTCATTTTGGAGG | mo- | |
| | | | GGAGGGTAATCACAAC | ccaataata | GTAATCACAAC | nu- | |
| | | | (SEQ ID. NO: 1929) | (SEQ ID. | (SEQ ID. | clease | |
| | | | | NO: | NO: 1931) | | |
| | | | | 1930) | | | |
| 7a | thermo_4 | CcaCas13b | atgctttgtttcaggtgtatcaacc | atgctttgttt | GTTGGAACTGCT | Ther- | 7a |
| | 0 | | aataaGTTGGAACTGCTCTCATTTT | caggtgtatc | CTCATTTTGGAGG | mo- | |
| | | | GGAGGGTAATCACAAC | aaccaataa | GTAATCACAAC | nu- | |
| | | | (SEQ ID. NO: 1932) | (SEQ ID. | (SEQ ID. | clease | |
| | | | | NO: | NO: 1934) | | |
| | | | | 1933) | | | |
| 7a | thermo_4 | CcaCas13b | ggatgctttgtttcaggtgtatcaa | ggatgctttg | GTTGGAACTGCT | Ther- | 7a |
| | 1 | | ccaatGTTGGAACTGCTCTCATTTT | tttcaggtgta | CTCATTTTGGAGG | mo- | |
| | | | GGAGGGTAATCACAAC | tcaaccaat | GTAATCACAAC | nu- | |
| | | | (SEQ ID. NO: 1935) | (SEQ ID. | (SEQ ID. | clease | |
| | | | | NO: | NO: 1937) | | |
| | | | | 1936) | | | |
| 7a | thermo_4 | CcaCas13b | taggatgctttgtttcaggtgtatc | taggatgcttt | GTTGGAACTGCT | Ther- | 7a |
| | 2 | | aaccaGTTGGAACTGCTCTCATTTT | gtttcaggtg | CTCATTTTGGAGG | mo- | |
| | | | GGAGGGTAATCACAAC | tatcaacca | GTAATCACAAC | nu- | |
| | | | (SEQ ID. NO: 1938) | (SEQ ID. | (SEQ ID. | clease | |
| | | | | NO: | NO: 1940) | | |
| | | | | 1939) | | | |
| 7a | thermo_4 | CcaCas13b | tttaggatgctttgtttcaggtgta | tttaggatgct | GTTGGAACTGCT | Ther- | 7a |
| | 3 | | tcaacGTTGGAACTGCTCTCATTTT | ttgtttcaggt | CTCATTTTGGAGG | mo- | |
| | | | GGAGGGTAATCACAAC | gtatcaac | GTAATCACAAC | nu- | |
| | | | (SEQ ID. NO: 1941) | (SEQ ID. | (SEQ ID. | clease | |
| | | | | NO: | NO: 1943) | | |
| | | | | 1942) | | | |
| 7a | thermo_4 | CcaCas13b | tttttaggatgctttgtttcaggtg | tttttaggatg | GTTGGAACTGCT | Ther- | 7a |
| | 4 | | tatcaGTTGGAACTGCTCTCATTTT | ctttgtttcag | CTCATTTTGGAGG | mo- | |
| | | | GGAGGGTAATCACAAC | gtgtatca | GTAATCACAAC | nu- | |
| | | | (SEQ ID. NO: 1944) | (SEQ ID. | (SEQ ID. | clease | |
| | | | | NO: | NO: 1946) | | |
| | | | | 1945) | | | |
| 7a | thermo_4 | CcaCas13b | cttttttaggatgctttgtttcagg | cttttttagga | GTTGGAACTGCT | Ther- | 7a |
| | 5 | | tgtatGTTGGAACTGCTCTCATTTT | tgctttgtttc | CTCATTTTGGAGG | mo- | |
| | | | GGAGGGTAATCACAAC | aggtgtat | GTAATCACAAC | nu- | |
| | | | (SEQ ID. NO: 1947) | (SEQ ID. | (SEQ ID. | clease | |
| | | | | NO: | NO: 1949) | | |
| | | | | 1948) | | | |
| 7a | thermo_4 | CcaCas13b | accttttttaggatgctttgtttca | accttttttag | GTTGGAACTGCT | Ther- | 7a |
| | 6 | | ggtgtGTTGGAACTGCTCTCATTTT | gatgctttgtt | CTCATTTTGGAGG | mo- | |
| | | | GGAGGGTAATCACAAC | tcaggtgt | GTAATCACAAC | nu- | |
| | | | (SEQ ID. NO: 1950) | (SEQ ID. | (SEQ ID. | clease | |
| | | | | NO: | NO: 1952) | | |
| | | | | 1951) | | | |
| 7a | thermo_4 | CcaCas13b | acaccttttttaggatgctttgttt | acacctttttt | GTTGGAACTGCT | Ther- | 7a |
| | 7 | | caggtGTTGGAACTGCTCTCATTTT | aggatgcttt | CTCATTTTGGAGG | mo- | |
| | | | GGAGGGTAATCACAAC | gtttcaggt | GTAATCACAAC | nu- | |
| | | | (SEQ ID. NO: 1953) | (SEQ ID. | (SEQ ID. | clease | |
| | | | | NO: | NO: 1955) | | |
| | | | | 1954) | | | |
| 7a | thermo_4 | CcaCas13b | ctacaccttttttaggatgctttgt | ctacacctttt | GTTGGAACTGCT | Ther- | 7a |
| | 8 | | ttcagGTTGGAACTGCTCTCATTTT | ttaggatgctt | CTCATTTTGGAGG | mo- | |
| | | | GGAGGGTAATCACAAC | tgtttcag | GTAATCACAAC | nu- | |
| | | | (SEQ ID. NO: 1956) | (SEQ ID. | (SEQ ID. | clease | |
| | | | | NO: | NO: 1958) | | |
| | | | | 1957) | | | |
| 7a | thermo_4 | CcaCas13b | ctctacaccttttttaggatgcttt | ctctacacctt | GTTGGAACTGCT | Ther- | 7a |
| | 9 | | gtttcGTTGGAACTGCTCTCATTTT | ttttaggatgc | CTCATTTTGGAGG | mo- | |
| | | | GGAGGGTAATCACAAC | tttgtttc | GTAATCACAAC | nu- | |
| | | | (SEQ ID. NO: 1959) | (SEQ ID. | (SEQ ID. | clease | |
| | | | | NO: | NO: 1961) | | |
| | | | | 1960) | | | |
| 7a | thermo_5 | CcaCas13b | ttctctacaccttttttaggatgct | ttctctacacc | GTTGGAACTGCT | Ther- | 7a |
| | 0 | | ttgttGTTGGAACTGCTCTCATTTT | ttttttaggat | CTCATTTTGGAGG | mo- | |
| | | | GGAGGGTAATCACAAC | gctttgtt | GTAATCACAAC | nu- | |
| | | | (SEQ ID. NO: 1962) | (SEQ ID. | (SEQ ID. | clease | |
| | | | | NO: | NO: 1964) | | |
| | | | | 1963) | | | |
| 7a | thermo_5 | CcaCas13b | atttctctacaccttttttaggatg | atttctctaca | GTTGGAACTGCT | Ther- | 7a |
| | 1 | | ctttgGTTGGAACTGCTCTCATTTT | ccttttttagg | CTCATTTTGGAGG | mo- | |
| | | | GGAGGGTAATCACAAC | atgctttg | GTAATCACAAC | nu- | |
| | | | (SEQ ID. NO: 1965) | (SEQ ID. | (SEQ ID. | clease | |
| | | | | NO: | NO: 1967) | | |
| | | | | 1966) | | | |
| 7a | thermo_5 | CcaCas13b | atatttctctacaccttttttagga | atatttctcta | GTTGGAACTGCT | Ther- | 7a |
| | 2 | | tgcttGTTGGAACTGCTCTCATTTT | cacctttttta | CTCATTTTGGAGG | mo- | |
| | | | GGAGGGTAATCACAAC | ggatgctt | GTAATCACAAC | nu- | |
| | | | (SEQ ID. NO: 1968) | (SEQ ID. | (SEQ ID. | clease | |
| | | | | NO: | NO: 1970) | | |
| | | | | 1969) | | | |
| 7a | thermo_5 | CcaCas13b | ccatatttctctacaccttttttag | ccatatttctc | GTTGGAACTGCT | Ther- | 7a |
| | 3 | | gatgcGTTGGAACTGCTCTCATTTT | tacaccttttt | CTCATTTTGGAGG | mo- | |
| | | | GGAGGGTAATCACAAC | taggatgc | GTAATCACAAC | nu- | |
| | | | (SEQ ID. NO: 1971) | (SEQ ID. | (SEQ ID. | clease | |
| | | | | NO: | NO: 1973) | | |
| | | | | 1972) | | | |
| 7a | thermo_5 | CcaCas13b | gaccatatttctctacacctttttt | gaccatattt | GTTGGAACTGCT | Ther- | 7a |
| | 4 | | aggatGTTGGAACTGCTCTCATTTT | ctctacacctt | CTCATTTTGGAGG | mo- | |
| | | | GGAGGGTAATCACAAC | ttttaggat | GTAATCACAAC | nu- | |
| | | | (SEQ ID. NO: 1974) | (SEQ ID. | (SEQ ID. | clease | |
| | | | | NO: | NO: 1976) | | |
| | | | | 1975) | | | |
| 7a | thermo_5 | CcaCas13b | aggaccatatttctctacacctttt | aggaccatat | GTTGGAACTGCT | Ther- | 7a |
| | 5 | | ttaggGTTGGAACTGCTCTCATTTT | ttctctacacc | CTCATTTTGGAGG | mo- | |
| | | | GGAGGGTAATCACAAC | ttttttagg | GTAATCACAAC | nu- | |
| | | | (SEQ ID. NO: 1977) | (SEQ ID. | (SEQ ID. | clease | |
| | | | | NO: | NO: 1979) | | |
| | | | | 1978) | | | |
| 7a | thermo_5 | CcaCas13b | tcaggaccatatttctctacacctt | tcaggaccat | GTTGGAACTGCT | Ther- | 7a |
| | 6 | | ttttaGTTGGAACTGCTCTCATTTT | atttctctaca | CTCATTTTGGAGG | mo- | |
| | | | GGAGGGTAATCACAAC | cctttttta | GTAATCACAAC | nu- | |
| | | | (SEQ ID. NO: 1980) | (SEQ ID. | (SEQ ID. | clease | |
| | | | | NO: | NO: 1982) | | |
| | | | | 1981) | | | |
| 7a | thermo_5 | CcaCas13b | cttcaggaccatatttctctacacc | cttcaggacc | GTTGGAACTGCT | Ther- | 7a |
| | 7 | | tttttGTTGGAACTGCTCTCATTTT | atatttctcta | CTCATTTTGGAGG | mo- | |
| | | | GGAGGGTAATCACAAC | caccttttt | GTAATCACAAC | nu- | |
| | | | (SEQ ID. NO: 1983) | (SEQ ID. | (SEQ ID. | clease | |
| | | | | NO: | NO: 1985) | | |
| | | | | 1984) | | | |
| 7a | thermo_5 | CcaCas13b | tgcttcaggaccatatttctctaca | tgcttcagga | GTTGGAACTGCT | Ther- | 7a |
| | 8 | | cctttGTTGGAACTGCTCTCATTTT | ccatatttctc | CTCATTTTGGAGG | mo- | |
| | | | GGAGGGTAATCACAAC | tacaccttt | GTAATCACAAC | nu- | |
| | | | (SEQ ID. NO: 1986) | (SEQ ID. | (SEQ ID. | clease | |
| | | | | NO: | NO: 1988) | | |
| | | | | 1987) | | | |
| 7a | thermo_5 | CcaCas13b | cttgcttcaggaccatatttctcta | cttgcttcag | GTTGGAACTGCT | Ther- | 7a |
| | 9 | | cacctGTTGGAACTGCTCTCATTTT | gaccatattt | CTCATTTTGGAGG | mo- | |
| | | | GGAGGGTAATCACAAC | ctctacacct | GTAATCACAAC | nu- | |
| | | | (SEQ ID. NO: 1989) | (SEQ ID. | (SEQ ID. | clease | |
| | | | | NO: | NO: 1991) | | |
| | | | | 1990) | | | |
| 7a | thermo_6 | CcaCas13b | cacttgcttcaggaccatatttctc | cacttgcttc | GTTGGAACTGCT | Ther- | 7a |
| | 0 | | tacacGTTGGAACTGCTCTCATTTT | aggaccatat | CTCATTTTGGAGG | mo- | |
| | | | GGAGGGTAATCACAAC | ttctctacac | GTAATCACAAC | nu- | |
| | | | (SEQ ID. NO: 1992) | (SEQ ID. | (SEQ ID. | clease | |
| | | | | NO: | NO: 1994) | | |
| | | | | 1993) | | | |
| 7a | thermo_6 | CcaCas13b | tgcacttgcttcaggaccatatttc | tgcacttgctt | GTTGGAACTGCT | Ther- | 7a |
| | 1 | | tctacGTTGGAACTGCTCTCATTTT | caggaccat | CTCATTTTGGAGG | mo- | |
| | | | GGAGGGTAATCACAAC | atttctctac | GTAATCACAAC | nu- | |
| | | | (SEQ ID. NO: 1995) | (SEQ ID. | (SEQ ID. | clease | |
| | | | | NO: | NO: 1997) | | |
| | | | | 1996) | | | |
| 7a | thermo_6 | CcaCas13b | aatgcacttgcttcaggaccatatt | aatgcacttg | GTTGGAACTGCT | Ther- | 7a |
| | 2 | | tctctGTTGGAACTGCTCTCATTTT | cttcaggacc | CTCATTTTGGAGG | mo- | |
| | | | GGAGGGTAATCACAAC | atatttctct | GTAATCACAAC | nu- | |
| | | | (SEQ ID. NO: 1998) | (SEQ ID. | (SEQ ID. | clease | |
| | | | | NO: | NO: 2000) | | |
| | | | | 1999) | | | |
| 7a | thermo_6 | CcaCas13b | taaatgcacttgcttcaggaccata | taaatgcact | GTTGGAACTGCT | Ther- | 7a |
| | 3 | | tttctGTTGGAACTGCTCTCATTTT | tgcttcagga | CTCATTTTGGAGG | mo- | |
| | | | GGAGGGTAATCACAAC | ccatatttct | GTAATCACAAC | nu- | |
| | | | (SEQ ID. NO: 2001) | (SEQ ID. | (SEQ ID. | clease | |
| | | | | NO: | NO: 2003) | | |
| | | | | 2002) | | | |
| 7a | thermo_6 | CcaCas13b | cgtaaatgcacttgcttcaggacca | cgtaaatgca | GTTGGAACTGCT | Ther- | 7a |
| | 4 | | tatttGTTGGAACTGCTCTCATTTT | cttgcttcag | CTCATTTTGGAGG | mo- | |
| | | | GGAGGGTAATCACAAC | gaccatattt | GTAATCACAAC | nu- | |
| | | | (SEQ ID. NO: 2004) | (SEQ ID. | (SEQ ID. | clease | |
| | | | | NO: | NO: 2006) | | |
| | | | | 2005) | | | |
| 7a | thermo_6 | CcaCas13b | tcgtaaatgcacttgcttcaggacc | tcgtaaatgc | GTTGGAACTGCT | Ther- | 7a |
| | 5 | | atattGTTGGAACTGCTCTCATTTT | acttgcttca | CTCATTTTGGAGG | mo- | |
| | | | GGAGGGTAATCACAAC | ggaccatatt | GTAATCACAAC | nu- | |
| | | | (SEQ ID. NO: 2007) | (SEQ ID. | (SEQ ID. | clease | |
| | | | | NO: | NO: 2009) | | |
| | | | | 2008) | | | |
| 7a | thermo_6 | CcaCas13b | ttcgtaaatgcacttgcttcaggac | ttcgtaaatg | GTTGGAACTGCT | Ther- | 7a |
| | 6 | | atatGTTGGAACTGCTCTCATTTT | cacttgcttc | CTCATTTTGGAGG | mo- | |
| | | | GcGAGGGTAATCACAAC | aggaccatat | GTAATCACAAC | nu- | |
| | | | (SEQ ID. NO: 2010) | (SEQ ID. | (SEQ ID. | clease | |
| | | | | NO: | NO: 2012) | | |
| | | | | 2011) | | | |
| 7a | thermo_6 | CcaCas13b | tttcgtaaatgcacttgcttcagga | tttcgtaaatg | GTTGGAACTGCT | Ther- | 7a |
| | 7 | | ccataGTTGGAACTGCTCTCATTTT | cacttgcttc | CTCATTTTGGAGG | mo- | |
| | | | GGAGGGTAATCACAAC | aggaccata | GTAATCACAAC | nu- | |
| | | | (SEQ ID. NO: 2013) | (SEQ ID. | (SEQ ID. | clease | |
| | | | | NO: | NO: 2015) | | |
| | | | | 2014) | | | |
| 7a | thermo_6 | CcaCas13b | ttttcgtaaatgcacttgcttcagg | ttttcgtaaat | GTTGGAACTGCT | Ther- | 7a |
| | 8 | | ccatGTTGGAACTGCTCTCATTTT | gcacttgctt | CTCATTTTGGAGG | mo- | |
| | | | aGGAGGGTAATCACAAC | caggaccat | GTAATCACAAC | nu- | |
| | | | (SEQ ID. NO: 2016) | (SEQ ID. | (SEQ ID. | clease | |
| | | | | NO: | NO: 2018) | | |
| | | | | 2017) | | | |
| 7a | thermo_6 | CcaCas13b | tttttcgtaaatgcacttgcttcag | tttttcgtaaa | GTTGGAACTGCT | Ther- | 7a |
| | 9 | | gaccaGTTGGAACTGCTCTCATTTT | tgcacttgctt | CTCATTTTGGAGG | mo- | |
| | | | GGAGGGTAATCACAAC | caggacca | GTAATCACAAC | nu- | |
| | | | (SEQ ID. NO: 2019) | (SEQ ID. | (SEQ ID. | clease | |
| | | | | NO: | NO: 2021) | | |
| | | | | 2020) | | | |
| 7a | thermo_7 | CcaCas13b | ctttttcgtaaatgcacttgcttca | ctttttcgtaa | GTTGGAACTGCT | Ther- | 7a |
| | 0 | | ggaccGTTGGAACTGCTCTCATTTT | atgcacttgc | CTCATTTTGGAGG | mo- | |
| | | | GGAGGGTAATCACAAC | ttcaggacc | GTAATCACAAC | nu- | |
| | | | (SEQ ID. NO: 2022) | (SEQ ID. | (SEQ ID. | clease | |
| | | | | NO: | NO: 2024) | | |
| | | | | 2023) | | | |
| 7a | thermo_7 | CcaCas13b | tctttttcgtaaatgcacttgcttc | tctttttcgta | GTTGGAACTGCT | Ther- | 7a |
| | 1 | | aggacGTTGGAACTGCTCTCATTTT | aatgcacttgc | CTCATTTTGGAGG | mo- | |
| | | | GGAGGGTAATCACAAC | ttcaggac | GTAATCACAAC | nu- | |
| | | | (SEQ ID. NO: 2025) | (SEQ ID. | (SEQ ID. | clease | |
| | | | | NO: | NO: 2027) | | |
| | | | | 2026) | | | |
| 7a | thermo_7 | CcaCas13b | atctttttcgtaaatgcacttgctt | atctttttcgt | GTTGGAACTGCT | Ther- | 7a |
| | 2 | | caggaGTTGGAACTGCTCTCATTTT | aaatgcacttg | CTCATTTTGGAGG | mo- | |
| | | | GGAGGGTAATCACAAC | cttcagga | GTAATCACAAC | nu- | |
| | | | (SEQ ID. NO: 2028) | (SEQ ID. | (SEQ ID. | clease | |
| | | | | NO: | NO: 2030) | | |
| | | | | 2029) | | | |
| 7a | thermo_7 | CcaCas13b | catctttttcgtaaatgcacttgct | catctttttcg | GTTGGAACTGCT | Ther- | 7a |
| | 3 | | tcaggGTTGGAACTGCTCTCATTTT | taaatgcactt | CTCATTTTGGAGG | mo- | |
| | | | GGAGGGTAATCACAAC | gcttcagg | GTAATCACAAC | nu- | |
| | | | (SEQ ID. NO: 2031) | (SEQ ID. | (SEQ ID. | clease | |
| | | | | NO: | NO: 2033) | | |
| | | | | 2032) | | | |
| 7a | thermo_7 | CcaCas13b | ccatctttttcgtaaatgcacttgc | ccatctttttc | GTTGGAACTGCT | Ther- | 7a |
| | 4 | | ttcagGTTGGAACTGCTCTCATTTT | gtaaatgcac | CTCATTTTGGAGG | mo- | |
| | | | GGAGGGTAATCACAAC | ttgcttcag | GTAATCACAAC | nu- | |
| | | | (SEQ ID. NO: 2034) | (SEQ ID. | (SEQ ID. | clease | |
| | | | | NO: | NO: 2036) | | |
| | | | | 2035) | | | |
| 7a | thermo_7 | CcaCas13b | accatctttttcgtaaatgcacttg | accatcttttt | GTTGGAACTGCT | Ther- | 7a |
| | 5 | | cttcaGTTGGAACTGCTCTCATTTT | cgtaaatgca | CTCATTTTGGAGG | mo- | |
| | | | GGAGGGTAATCACAAC | cttgcttca | GTAATCACAAC | nu- | |
| | | | (SEQ ID. NO: 2037) | (SEQ ID. | (SEQ ID. | clease | |
| | | | | NO: | NO: 2039) | | |
| | | | | 2038) | | | |
| 7a | thermo_7 | CcaCas13b | taccatctttttcgtaaatgcactt | taccatctttt | GTTGGAACTGCT | Ther- | 7a |
| | 6 | | gcttcGTTGGAACTGCTCTCATTTT | tcgtaaatgca | CTCATTTTGGAGG | mo- | |
| | | | GGAGGGTAATCACAAC | cttgcttc | GTAATCACAAC | nu- | |
| | | | (SEQ ID. NO: 2040) | (SEQ ID. | (SEQ ID. | clease | |
| | | | | NO: | NO: 2042) | | |
| | | | | 2041) | | | |
| 7a | thermo_7 | CcaCas13b | ctaccatctttttcgtaaatgcact | ctaccatcttt | GTTGGAACTGCT | Ther- | 7a |
| | 7 | | gtcttGTTGGAACTGCTCTCATTTT | ttcgtaaatg | CTCATTTTGGAGG | mo- | |
| | | | GGAGGGTAATCACAAC | cacttgctt | GTAATCACAAC | nu- | |
| | | | (SEQ ID. NO: 2043) | (SEQ ID. | (SEQ ID. | clease | |
| | | | | NO: | NO: 2045) | | |
| | | | | 2044) | | | |
| 7a | thermo_7 | CcaCas13b | tctaccatctttttcgtaaatgcac | tctaccatctt | GTTGGAACTGCT | Ther- | 7a |
| | 8 | | ttgctGTTGGAACTGCTCTCATTTT | tttcgtaaatg | CTCATTTTGGAGG | mo- | |
| | | | GGAGGGTAATCACAAC | cacttgct | GTAATCACAAC | nu- | |
| | | | (SEQ ID. NO: 2046) | (SEQ ID. | (SEQ ID. | clease | |
| | | | | NO: | NO: 2048) | | |
| | | | | 2047) | | | |
| 7a | thermo_7 | CcaCas13b | ttctaccatctttttcgtaaatgca | ttctaccatct | GTTGGAACTGCT | Ther- | 7a |
| | 9 | | ttgcGTTGGAACTGCTCTCATTTT | ttttcgtaaat | CTCATTTTGGAGG | mo- | |
| | | | GcGAGGGTAATCACAAC | gcacttgc | GTAATCACAAC | nu- | |
| | | | (SEQ ID. NO: 2049) | (SEQ ID. | (SEQ ID. | clease | |
| | | | | NO: | NO: 2051) | | |
| | | | | 2050) | | | |
| 7a | thermo_8 | CcaCas13b | tttctaccatctttttcgtaaatgc | tttctaccatc | GTTGGAACTGCT | Ther- | 7a |
| | 0 | | acttgGTTGGAACTGCTCTCATTTT | tttttcgtaaa | CTCATTTTGGAGG | mo- | |
| | | | GGAGGGTAATCACAAC | tgcacttg | GTAATCACAAC | nu- | |
| | | | (SEQ ID. NO: 2052) | (SEQ ID. | (SEQ ID. | clease | |
| | | | | NO: | NO: 2054) | | |
| | | | | 2053) | | | |
| 7a | thermo_8 | CcaCas13b | ttttctaccatctttttcgtaaatg | ttttctaccat | GTTGGAACTGCT | Ther- | 7a |
| | 1 | | cacttGTTGGAACTGCTCTCATTTT | ctttttcgtaa | CTCATTTTGGAGG | mo- | |
| | | | GGAGGGTAATCACAAC | atgcactt | GTAATCACAAC | nu- | |
| | | | (SEQ ID. NO: 2055) | (SEQ ID. | (SEQ ID. | clease | |
| | | | | NO: | NO: 2057) | | |
| | | | | 2056) | | | |
| 7a | thermo_8 | CcaCas13b | attttctaccatctttttcgtaaat | attttctacca | GTTGGAACTGCT | Ther- | 7a |
| | 2 | | gcactGTTGGAACTGCTCTCATTTT | tctttttcgta | CTCATTTTGGAGG | mo- | |
| | | | GGAGGGTAATCACAAC | aatgcact | GTAATCACAAC | nu- | |
| | | | (SEQ ID. NO: 2058) | (SEQ ID. | (SEQ ID. | clease | |
| | | | | NO: | NO: 2060) | | |
| | | | | 2059) | | | |
| 7a | thermo_8 | CcaCas13b | cattttctaccatctttttcgtaaa | cattttctacc | GTTGGAACTGCT | Ther- | 7a |
| | 3 | | tgcacGTTGGAACTGCTCTCATTTT | atctttttcgt | CTCATTTTGGAGG | mo- | |
| | | | GGAGGGTAATCACAAC | aaatgcac | GTAATCACAAC | nu- | |
| | | | (SEQ ID. NO: 2061) | (SEQ ID. | (SEQ ID. | clease | |
| | | | | NO: | NO: 2063) | | |
| | | | | 2062) | | | |
| 7a | thermo_8 | CcaCas13b | gcattttctaccatctttttcgtaa | gcattttctac | GTTGGAACTGCT | Ther- | 7a |
| | 4 | | atgcaGTTGGAACTGCTCTCATTTT | catctttttcg | CTCATTTTGGAGG | mo- | |
| | | | GGAGGGTAATCACAAC | taaatgca | GTAATCACAAC | nu- | |
| | | | (SEQ ID. NO: 2064) | (SEQ ID. | (SEQ ID. | clease | |
| | | | | NO: | NO: 2066) | | |
| | | | | 2065) | | | |
| 7a | thermo_8 | CcaCas13b | tgcattttctaccatctttttcgta | tgcattttcta | GTTGGAACTGCT | Ther- | 7a |
| | 5 | | aatgcGTTGGAACTGCTCTCATTTT | ccatctttttc | CTCATTTTGGAGG | mo- | |
| | | | GGAGGGTAATCACAAC | gtaaatgc | GTAATCACAAC | nu- | |
| | | | (SEQ ID. NO: 2067) | (SEQ ID. | (SEQ ID. | clease | |
| | | | | NO: | NO: 2069) | | |
| | | | | 2068) | | | |
| 7a | thermo_8 | CcaCas13b | ttgcattttctaccatctttttcgt | ttgcattttct | GTTGGAACTGCT | Ther- | 7a |
| | 6 | | aaatgGTTGGAACTGCTCTCATTTT | accatcttttt | CTCATTTTGGAGG | mo- | |
| | | | GGAGGGTAATCACAAC | cgtaaatg | GTAATCACAAC | nu- | |
| | | | (SEQ ID. NO: 2070) | (SEQ ID. | (SEQ ID. | clease | |
| | | | | NO: | NO: 2072) | | |
| | | | | 2071) | | | |
| 7a | thermo_8 | CcaCas13b | tttgcattttctaccatctttttcg | tttgcattttc | GTTGGAACTGCT | Ther- | 7a |
| | 7 | | taaatGTTGGAACTGCTCTCATTTT | taccatctttt | CTCATTTTGGAGG | mo- | |
| | | | GGAGGGTAATCACAAC | tcgtaaat | GTAATCACAAC | nu- | |
| | | | (SEQ ID. NO: 2073) | (SEQ ID. | (SEQ ID. | clease | |
| | | | | NO: | NO: 2075) | | |
| | | | | 2074) | | | |
| 7a | thermo_8 | CcaCas13b | ctttgcattttctaccatctttttc | ctttgcatttt | GTTGGAACTGCT | Ther- | 7a |
| | 8 | | gtaaaGTTGGAACTGCTCTCATTTT | ctaccatcttt | CTCATTTTGGAGG | mo- | |
| | | | GGAGGGTAATCACAAC | ttcgtaaa | GTAATCACAAC | nu- | |
| | | | (SEQ ID. NO: 2076) | (SEQ ID. | (SEQ ID. | clease | |
| | | | | NO: | NO: 2078) | | |
| | | | | 2077) | | | |
| 7a | thermo_8 | CcaCas13b | tctttgcattttctaccatcttttt | tctttgcattt | GTTGGAACTGCT | Ther- | 7a |
| | 9 | | cgtaaGTTGGAACTGCTCTCATTTT | tctaccatctt | CTCATTTTGGAGG | mo- | |
| | | | GGAGGGTAATCACAAC | tttcgtaa | GTAATCACAAC | nu- | |
| | | | (SEQ ID. NO: 2079) | (SEQ ID. | (SEQ ID. | clease | |
| | | | | NO: | NO: 2081) | | |
| | | | | 2080) | | | |
| 7a | thermo_9 | CcaCas13b | ttctttgcattttctaccatctttt | ttctttgcatt | GTTGGAACTGCT | Ther- | 7a |
| | 0 | | tcgtaGTTGGAACTGCTCTCATTTT | ttctaccatct | CTCATTTTGGAGG | mo- | |
| | | | GGAGGGTAATCACAAC | ttttcgta | GTAATCACAAC | nu- | |
| | | | (SEQ ID. NO: 2082) | (SEQ ID. | (SEQ ID. | clease | |
| | | | | NO: | NO: 2084) | | |
| | | | | 2083) | | | |
| 7a | thermo_9 | CcaCas13b | tttctttgcattttctaccatcttt | tttctttgcat | GTTGGAACTGCT | Ther- | 7a |
| | 1 | | ttcgtGTTGGAACTGCTCTCATTTT | tttctaccatc | CTCATTTTGGAGG | mo- | |
| | | | GGAGGGTAATCACAAC | tttttcgt | GTAATCACAAC | nu- | |
| | | | (SEQ ID. NO: 2085) | (SEQ ID. | (SEQ ID. | clease | |
| | | | | NO: | NO: 2087) | | |
| | | | | 2086) | | | |
| 7a | thermo_9 | CcaCas13b | ttttctttgcattttctaccatctt | ttttctttgca | GTTGGAACTGCT | Ther- | 7a |
| | 2 | | tttcgGTTGGAACTGCTCTCATTTT | ttttctaccat | CTCATTTTGGAGG | mo- | |
| | | | GGAGGGTAATCACAAC | ctttttcg | GTAATCACAAC | nu- | |
| | | | (SEQ ID. NO: 2088) | (SEQ ID. | (SEQ ID. | clease | |
| | | | | NO: | NO: 2090) | | |
| | | | | 2089) | | | |
| 7a | thermo_9 | CcaCas13b | attttctttgcattttctaccatct | attttctttgc | GTTGGAACTGCT | Ther- | 7a |
| | 3 | | ttttcGTTGGAACTGCTCTCATTTT | attttctacca | CTCATTTTGGAGG | mo- | |
| | | | GGAGGGTAATCACAAC | tctttttc | GTAATCACAAC | nu- | |
| | | | (SEQ ID. NO: 2091) | (SEQ ID. | (SEQ ID. | clease | |
| | | | | NO: | NO: 2093) | | |
| | | | | 2092) | | | |
| 7a | thermo_9 | CcaCas13b | aattttctttgcattttctaccatc | aattttctttg | GTTGGAACTGCT | Ther- | 7a |
| | 4 | | tttttGTTGGAACTGCTCTCATTTT | cattttctacc | CTCATTTTGGAGG | mo- | |
| | | | GGAGGGTAATCACAAC | atcttttt | GTAATCACAAC | nu- | |
| | | | (SEQ ID. NO: 2094) | (SEQ ID. | (SEQ ID. | clease | |
| | | | | NO: | NO: 2096) | | |
| | | | | 2095) | | | |
| 7a | thermo_9 | CcaCas13b | caattttctttgcattttctaccat | caattttcttt | GTTGGAACTGCT | Ther- | 7a |
| | 5 | | cttttGTTGGAACTGCTCTCATTTT | gcattttctac | CTCATTTTGGAGG | mo- | |
| | | | GGAGGGTAATCACAAC | catctttt | GTAATCACAAC | nu- | |
| | | | (SEQ ID. NO: 2097) | (SEQ ID. | (SEQ ID. | clease | |
| | | | | NO: | NO: 2099) | | |
| | | | | 2098) | | | |
| 7a | ssrna1_0 | CcaCas13b | atccccgggtaccgagctcgaattc | atccccggg | GTTGGAACTGCT | ssRNA1 | 7a |
| | | | actggGTTGGAACTGCTCTCATTT | taccgagctc | CTCATTTTGGAGG | | |
| | | | TGGAGGGTAATCACAAC | gaattcactg | GTAATCACAAC | | |
| | | | (SEQ ID. NO: 2100) | g (SEQ | (SEQ ID. | | |
| | | | | ID. NO: | NO: 2102) | | |
| | | | | 2101) | | | |
| 7a | ssrna1_1 | CcaCas13b | gatccccgggtaccgagctcgaatt | gatccccgg | GTTGGAACTGCT | ssRNA1 | 7a |
| | | | cactgGTTGGAACTGCTCTCATTTT | gtaccgagc | CTCATTTTGGAGG | | |
| | | | GGAGGGTAATCACAAC | tcgaattcac | GTAATCACAAC | | |
| | | | (SEQ ID. NO: 2103) | tg (SEQ | (SEQ ID. | | |
| | | | | ID. NO: | NO: 2105) | | |
| | | | | 2104) | | | |
| 7a | ssrna1_2 | CcaCas13b | ggatccccgggtaccgagctcgaat | ggatccccg | GTTGGAACTGCT | ssRNA1 | 7a |
| | | | tcactGTTGGAACTGCTCTCATTTT | ggtaccgag | CTCATTTTGGAGG | | |
| | | | GGAGGGTAATCACAAC | ctcgaattca | GTAATCACAAC | | |
| | | | (SEQ ID. NO: 2106) | ct (SEQ | (SEQ ID. | | |
| | | | | ID. NO: | NO: 2108) | | |
| | | | | 2107) | | | |
| 7a | ssrna1_3 | CcaCas13b | agaggatccccgggtaccgagctcg | agaggatcc | GTTGGAACTGCT | ssRNA1 | 7a |
| | | | aattcGTTGGAACTGCTCTCATTTT | ccgggtacc | CTCATTTTGGAGG | | |
| | | | GGAGGGTAATCACAAC | gagctcgaa | GTAATCACAAC | | |
| | | | (SEQ ID. NO: 2109) | ttc (SEQ | (SEQ ID. | | |
| | | | | ID. NO: | NO: 2111) | | |
| | | | | 2110) | | | |
| 7a | ssrna1_4 | CcaCas13b | ctagaggatccccgggtaccgagct | ctagaggat | GTTGGAACTGCT | ssRNA1 | 7a |
| | | | cgaatGTTGGAACTGCTCTCATTTT | ccccgggta | CTCATTTTGGAGG | | |
| | | | GGAGGGTAATCACAAC | ccgagctcg | GTAATCACAAC | | |
| | | | (SEQ ID. NO: 2112) | aat (SEQ | (SEQ ID. | | |
| | | | | ID. NO: | NO: 2114) | | |
| | | | | 2113) | | | |
| 7a | ssrna1_5 | CcaCas13b | tttctagaggatccccgggtaccga | tttctagagg | GTTGGAACTGCT | ssRNA1 | 7a |
| | | | gctcgGTTGGAACTGCTCTCATTTT | atccccggg | CTCATTTTGGAGG | | |
| | | | GGAGGGTAATCACAAC | taccgagctc | GTAATCACAAC | | |
| | | | (SEQ ID. NO: 2115) | g (SEQ | (SEQ ID. | | |
| | | | | ID. NO: | NO: 2117) | | |
| | | | | 2116) | | | |
| 7a | ssrna1_6 | CcaCas13b | atttctagaggatccccgggtaccg | atttctagag | GTTGGAACTGCT | ssRNA1 | 7a |
| | | | agctcGTTGGAACTGCTCTCATTTT | gatccccgg | CTCATTTTGGAGG | | |
| | | | GGAGGGTAATCACAAC | gtaccgagc | GTAATCACAAC | | |
| | | | (SEQ ID. NO: 2118) | tc (SEQ | (SEQ ID. | | |
| | | | | ID. NO: | NO: 2120) | | |
| | | | | 2119) | | | |
| 7a | ssrna1_7 | CcaCas13b | atatttctagaggatccccgggtac | atatttctaga | GTTGGAACTGCT | ssRNA1 | 7a |
| | | | cgagcGTTGGAACTGCTCTCATTTT | ggatccccg | CTCATTTTGGAGG | | |
| | | | GGAGGGTAATCACAAC | ggtaccgag | GTAATCACAAC | | |
| | | | (SEQ ID. NO: 2121) | c (SEQ | (SEQ ID. | | |
| | | | | ID. NO: | NO: 2123) | | |
| | | | | 2122) | | | |
| 7a | ssrna1_8 | CcaCas13b | catatttctagaggatccccgggta | catatttctag | GTTGGAACTGCT | ssRNA1 | 7a |
| | | | ccgagGTTGGAACTGCTCTCATTTT | aggatcccc | CTCATTTTGGAGG | | |
| | | | GGAGGGTAATCACAAC | gggtaccga | GTAATCACAAC | | |
| | | | (SEQ ID. NO: 2124) | g (SEQ | (SEQ ID. | | |
| | | | | ID. NO: | NO: 2126) | | |
| | | | | 2125) | | | |
| 7a | ssrna1_9 | CcaCas13b | atccatatttctagaggatccccgg | atccatatttc | GTTGGAACTGCT | ssRNA1 | 7a |
| | | | gtaccGTTGGAACTGCTCTCATTTT | tagaggatc | CTCATTTTGGAGG | | |
| | | | GGAGGGTAATCACAAC | cccgggtac | GTAATCACAAC | | |
| | | | (SEQ ID. NO: 2127) | c (SEQ | (SEQ ID. | | |
| | | | | ID. NO: | NO: 2129) | | |
| | | | | 2128) | | | |
| 7a | ssrna1_10 | CcaCas13b | aatccatatttctagaggatccccg | aatccatattt | GTTGGAACTGCT | ssRNA1 | 7a |
| | | | ggtacGTTGGAACTGCTCTCATTTT | ctagaggat | CTCATTTTGGAGG | | |
| | | | GGAGGGTAATCACAAC | ccccgggta | GTAATCACAAC | | |
| | | | (SEQ ID. NO: 2130) | c (SEQ | (SEQ ID. | | |
| | | | | ID. NO: | NO: 2132) | | |
| | | | | 2131) | | | |
| 7a | ssrna1_11 | CcaCas13b | taatccatatttctagaggatcccc | taatccatatt | GTTGGAACTGCT | ssRNA1 | 7a |
| | | | gggtaGTTGGAACTGCTCTCATTTT | tctagaggat | CTCATTTTGGAGG | | |
| | | | GGAGGGTAATCACAAC | ccccgggta | GTAATCACAAC | | |
| | | | (SEQ ID. NO: 2133) | (SEQ ID. | (SEQ ID. | | |
| | | | | NO: | NO: 2135) | | |
| | | | | 2134) | | | |
| 7a | ssrna1_12 | CcaCas13b | agtaatccatatttctagaggatcc | agtaatccat | GTTGGAACTGCT | ssRNA1 | 7a |
| | | | ccgggGTTGGAACTGCTCTCATTTT | atttctagag | CTCATTTTGGAGG | | |
| | | | GGAGGGTAATCACAAC | gatccccgg | GTAATCACAAC | | |
| | | | (SEQ ID. NO: 2136) | g (SEQ | (SEQ ID. | | |
| | | | | ID. NO: | NO: 2138) | | |
| | | | | 2137) | | | |
| 7a | ssrna1_13 | CcaCas13b | aagtaatccatatttctagaggatc | aagtaatcca | GTTGGAACTGCT | ssRNA1 | 7a |
| | | | cccggGTTGGAACTGCTCTCATTTT | tatttctagag | CTCATTTTGGAGG | | |
| | | | GGAGGGTAATCACAAC | gatccccgg | GTAATCACAAC | | |
| | | | (SEQ ID. NO: 2139) | (SEQ ID. | (SEQ ID. | | |
| | | | | NO: | NO: 2141) | | |
| | | | | 2140) | | | |
| 7a | ssrna1_14 | CcaCas13b | tctaccaagtaatccatatttctag | tctaccaagt | GTTGGAACTGCT | ssRNA1 | 7a |
| | | | aggatGTTGGAACTGCTCTCATTTT | aatccatattt | CTCATTTTGGAGG | | |
| | | | GGAGGGTAATCACAAC | ctagaggat | GTAATCACAAC | | |
| | | | (SEQ ID. NO: 2142) | (SEQ ID. | (SEQ ID. | | |
| | | | | NO: | NO: 2144) | | |
| | | | | 2143) | | | |
| 7a | ssrna1_15 | CcaCas13b | gttctaccaagtaatccatatttct | gttctaccaa | GTTGGAACTGCT | ssRNA1 | 7a |
| | | | agaggGTTGGAACTGCTCTCATTTT | gtaatccata | CTCATTTTGGAGG | | |
| | | | GGAGGGTAATCACAAC | tttctagagg | GTAATCACAAC | | |
| | | | (SEQ ID. NO: 2145) | (SEQ ID. | (SEQ ID. | | |
| | | | | NO: | NO: 2147) | | |
| | | | | 2146) | | | |
| 7a | ssrna1_16 | CcaCas13b | ctgttctaccaagtaatccatattt | ctgttctacc | GTTGGAACTGCT | ssRNA1 | 7a |
| | | | ctagaGTTGGAACTGCTCTCATTTT | aagtaatcca | CTCATTTTGGAGG | | |
| | | | GGAGGGTAATCACAAC | tatttctaga | GTAATCACAAC | | |
| | | | (SEQ ID. NO: 2148) | (SEQ ID. | (SEQ ID. | | |
| | | | | NO: | NO: 2150) | | |
| | | | | 2149) | | | |
| 7a | ssrna1_17 | CcaCas13b | ttgctgttctaccaagtaatccata | ttgctgttcta | GTTGGAACTGCT | ssRNA1 | 7a |
| | | | tttctGTTGGAACTGCTCTCATTTT | ccaagtaatc | CTCATTTTGGAGG | | |
| | | | GGAGGGTAATCACAAC | catatttct | GTAATCACAAC | | |
| | | | (SEQ ID. NO: 2151) | (SEQ ID. | (SEQ ID. | | |
| | | | | NO: | NO: 2153) | | |
| | | | | 2152) | | | |
| 7a | ssrna1_18 | CcaCas13b | gattgctgttctaccaagtaatcca | gattgctgttc | GTTGGAACTGCT | ssRNA1 | 7a |
| | | | tatttGTTGGAACTGCTCTCATTTT | taccaagtaa | CTCATTTTGGAGG | | |
| | | | GGAGGGTAATCACAAC | tccatattt | GTAATCACAAC | | |
| | | | (SEQ ID. NO: 2154) | (SEQ ID. | (SEQ ID. | | |
| | | | | NO: | NO: 2156) | | |
| | | | | 2155) | | | |
| 7a | ssrna1_19 | CcaCas13b | agattgctgttctaccaagtaatcc | agattgctgtt | GTTGGAACTGCT | ssRNA1 | 7a |
| | | | tattGTTGGAACTGCTCTCATTTT | ctaccaagta | CTCATTTTGGAGG | | |
| | | | aGGAGGGTAATCACAAC | atccatatt | GTAATCACAAC | | |
| | | | (SEQ ID. NO: 2157) | (SEQ ID. | (SEQ ID. | | |
| | | | | NO: | NO: 2159) | | |
| | | | | 2158) | | | |
| 7a | ssrna1_20 | CcaCas13b | agtagattgctgttctaccaagtaa | agtagattgc | GTTGGAACTGCT | ssRNA1 | 7a |
| | | | tccatGTTGGAACTGCTCTCATTTT | tgttctacca | CTCATTTTGGAGG | | |
| | | | GGAGGGTAATCACAAC | agtaatccat | GTAATCACAAC | | |
| | | | (SEQ ID. NO: 2160) | (SEQ ID. | (SEQ ID. | | |
| | | | | NO: | NO: 2162) | | |
| | | | | 2161) | | | |
| 7a | ssrna1_21 | CcaCas13b | gagtagattgctgttctaccaagta | gagtagattg | GTTGGAACTGCT | ssRNA1 | 7a |
| | | | atccaGTTGGAACTGCTCTCATTTT | ctgttctacc | CTCATTTTGGAGG | | |
| | | | GGAGGGTAATCACAAC | aagtaatcca | GTAATCACAAC | | |
| | | | (SEQ ID. NO: 2163) | (SEQ ID. | (SEQ ID. | | |
| | | | | NO: | NO: 2165) | | |
| | | | | 2164) | | | |
| 7a | ssrna1_22 | CcaCas13b | cgagtagattgctgttctaccaagt | cgagtagatt | GTTGGAACTGCT | ssRNA1 | 7a |
| | | | aTatccGTGGAACTGCTCTCATTTT | gctgttctac | CTCATTTTGGAGG | | |
| | | | GGAGGGTAATCACAAC | caagtaatcc | GTAATCACAAC | | |
| | | | (SEQ ID. NO: 2166) | (SEQ ID. | (SEQ ID. | | |
| | | | | NO: | NO: 2168) | | |
| | | | | 2167) | | | |
| 7a | ssrna1_23 | CcaCas13b | tcgagtagattgctgttctaccaag | tcgagtagat | GTTGGAACTGCT | ssRNA1 | 7a |
| | | | tTaatcGTGGAACTGCTCTCATTTT | tgctgttctac | CTCATTTTGGAGG | | |
| | | | GGAGGGTAATCACAAC | caagtaatc | GTAATCACAAC | | |
| | | | (SEQ ID. NO: 2169) | (SEQ ID. | (SEQ ID. | | |
| | | | | NO: | NO: 2171) | | |
| | | | | 2170) | | | |
| 7a | ssrna1_24 | CcaCas13b | ggtcgagtagattgctgttctacca | ggtcgagta | GTTGGAACTGCT | ssRNA1 | 7a |
| | | | agtaaGTTGGAACTGCTCTCATTTT | gattgctgttc | CTCATTTTGGAGG | | |
| | | | GGAGGGTAATCACAAC | taccaagtaa | GTAATCACAAC | | |
| | | | (SEQ ID. NO: 2172) | (SEQ ID. | (SEQ ID. | | |
| | | | | NO: | NO: 2174) | | |
| | | | | 2173) | | | |
| 7a | ssrna1_25 | CcaCas13b | aggtcgagtagattgctgttctacc | aggtcgagt | GTTGGAACTGCT | ssRNA1 | 7a |
| | | | aagtaGTTGGAACTGCTCTCATTTT | agattgctgtt | CTCATTTTGGAGG | | |
| | | | GGAGGGTAATCACAAC | ctaccaagta | GTAATCACAAC | | |
| | | | (SEQ ID. NO: 2175) | (SEQ ID. | (SEQ ID. | | |
| | | | | NO: | NO: 2177) | | |
| | | | | 2176) | | | |
| 7a | ssrna1_26 | CcaCas13b | gcaggtcgagtagattgctgttcta | gcaggtcga | GTTGGAACTGCT | ssRNA1 | 7a |
| | | | ccaagGTTGGAACTGCTCTCATTTT | gtagattgct | CTCATTTTGGAGG | | |
| | | | GGAGGGTAATCACAAC | gttctaccaa | GTAATCACAAC | | |
| | | | (SEQ ID. NO: 2178) | g (SEQ | (SEQ ID. | | |
| | | | | ID. NO: | NO: 2180) | | |
| | | | | 2179) | | | |
| 7a | ssrna1_27 | CcaCas13b | tgcaggtcgagtagattgctgttct | tgcaggtcg | GTTGGAACTGCT | ssRNA1 | 7a |
| | | | accaaGTTGGAACTGCTCTCATTTT | agtagattgc | CTCATTTTGGAGG | | |
| | | | GGAGGGTAATCACAAC | tgttctacca | GTAATCACAAC | | |
| | | | (SEQ ID. NO: 2181) | a (SEQ | (SEQ ID. | | |
| | | | | ID. NO: | NO: 2183) | | |
| | | | | 2182) | | | |
| 7a | ssrna1_28 | CcaCas13b | ctgcaggtcgagtagattgctgttc | ctgcaggtc | GTTGGAACTGCT | ssRNA1 | 7a |
| | | | taccaGTTGGAACTGCTCTCATTTT | gagtagattg | CTCATTTTGGAGG | | |
| | | | GGAGGGTAATCACAAC | ctgttctacc | GTAATCACAAC | | |
| | | | (SEQ ID. NO: 2184) | a (SEQ | (SEQ ID. | | |
| | | | | ID. NO: | NO: 2186) | | |
| | | | | 2185) | | | |
| 7a | ssrna1_29 | CcaCas13b | cctgcaggtcgagtagattgctgtt | cctgcaggt | GTTGGAACTGCT | ssRNA1 | 7a |
| | | | ctaccGTTGGAACTGCTCTCATTTT | cgagtagatt | CTCATTTTGGAGG | | |
| | | | GGAGGGTAATCACAAC | gctgttctac | GTAATCACAAC | | |
| | | | (SEQ ID. NO: 2187) | c (SEQ | (SEQ ID. | | |
| | | | | ID. NO: | NO: 2189) | | |
| | | | | 2188) | | | |
| 7a | ssrna1_30 | CcaCas13b | gcctgcaggtcgagtagattgctgt | gcctgcagg | GTTGGAACTGCT | ssRNA1 | 7a |
| | | | tctacGTTGGAACTGCTCTCATTTT | tcgagtagat | CTCATTTTGGAGG | | |
| | | | GGAGGGTAATCACAAC | tgctgttctac | GTAATCACAAC | | |
| | | | (SEQ ID. NO: 2190) | (SEQ ID. | (SEQ ID. | | |
| | | | | NO: | NO: 2192) | | |
| | | | | 2191) | | | |
| 7a | ssrna1_31 | CcaCas13b | tgcctgcaggtcgagtagattgctg | tgcctgcag | GTTGGAACTGCT | ssRNA1 | 7a |
| | | | ttctaGTTGGAACTGCTCTCATTTT | gtcgagtag | CTCATTTTGGAGG | | |
| | | | GGAGGGTAATCACAAC | attgctgttct | GTAATCACAAC | | |
| | | | (SEQ ID. NO: 2193) | a (SEQ | (SEQ ID. | | |
| | | | | ID. NO: | NO: 2195) | | |
| | | | | 2194) | | | |
| 7a | ssrna1_32 | CcaCas13b | catgcctgcaggtcgagtagattgc | catgcctgca | GTTGGAACTGCT | ssRNA1 | 7a |
| | | | tgttcGTTGGAACTGCTCTCATTTT | ggtcgagta | CTCATTTTGGAGG | | |
| | | | GGAGGGTAATCACAAC | gattgctgttc | GTAATCACAAC | | |
| | | | (SEQ ID. NO: 2196) | (SEQ ID. | (SEQ ID. | | |
| | | | | NO: | NO: 2198) | | |
| | | | | 2197) | | | |
| 7a | ssrna1_33 | CcaCas13b | gcatgcctgcaggtcgagtagattg | gcatgcctg | GTTGGAACTGCT | ssRNA1 | 7a |
| | | | ctgttGTTGGAACTGCTCTCATTTT | caggtcgag | CTCATTTTGGAGG | | |
| | | | GGAGGGTAATCACAAC | tagattgctgt | GTAATCACAAC | | |
| | | | (SEQ ID. NO: 2199) | t (SEQ | (SEQ ID. | | |
| | | | | ID. NO: | NO: 2201) | | |
| | | | | 2200) | | | |
| 7a | ssrna1_34 | CcaCas13b | tgcatgcctgcaggtcgagtagatt | tgcatgcctg | GTTGGAACTGCT | ssRNA1 | 7a |
| | | | gctgtGTTGGAACTGCTCTCATTTT | caggtcgag | CTCATTTTGGAGG | | |
| | | | GGAGGGTAATCACAAC | tagattgctgt | GTAATCACAAC | | |
| | | | (SEQ ID. NO: 2202) | (SEQ ID. | (SEQ ID. | | |
| | | | | NO: | NO: 2204) | | |
| | | | | 2203) | | | |
| 7a | ssrna1_35 | CcaCas13b | cttgcatgcctgcaggtcgagtaga | cttgcatgcc | GTTGGAACTGCT | ssRNA1 | 7a |
| | | | ttgctGTTGGAACTGCTCTCATTTT | tgcaggtcg | CTCATTTTGGAGG | | |
| | | | GGAGGGTAATCACAAC | agtagattgc | GTAATCACAAC | | |
| | | | (SEQ ID. NO: 2205) | t (SEQ | (SEQ ID. | | |
| | | | | ID. NO: | NO: 2207) | | |
| | | | | 2206) | | | |
| 7a | ssrna1_36 | CcaCas13b | gcttgcatgcctgcaggtcgagtag | gcttgcatgc | GTTGGAACTGCT | ssRNA1 | 7a |
| | | | attgcGTTGGAACTGCTCTCATTTT | ctgcaggtc | CTCATTTTGGAGG | | |
| | | | GGAGGGTAATCACAAC | gagtagattg | GTAATCACAAC | | |
| | | | (SEQ ID. NO: 2208) | c (SEQ | (SEQ ID. | | |
| | | | | ID. NO: | NO: 2210) | | |
| | | | | 2209) | | | |
| 7a | ssrna1_37 | CcaCas13b | agcttgcatgcctgcaggtcgagta | agcttgcatg | GTTGGAACTGCT | ssRNA1 | 7a |
| | | | gattgGTTGGAACTGCTCTCATTTT | cctgcaggt | CTCATTTTGGAGG | | |
| | | | GGAGGGTAATCACAAC | cgagtagatt | GTAATCACAAC | | |
| | | | (SEQ ID. NO: 2211) | g (SEQ | (SEQ ID. | | |
| | | | | ID. NO: | NO: 2213) | | |
| | | | | 2212) | | | |
| 7a | ssrna1_38 | CcaCas13b | aagcttgcatgcctgcaggtcgagt | aagcttgcat | GTTGGAACTGCT | ssRNA1 | 7a |
| | | | agattGTTGGAACTGCTCTCATTTT | gcctgcagg | CTCATTTTGGAGG | | |
| | | | GGAGGGTAATCACAAC | tcgagtagat | GTAATCACAAC | | |
| | | | (SEQ ID. NO: 2214) | t (SEQ | (SEQ ID. | | |
| | | | | ID. NO: | NO: 2216) | | |
| | | | | 2215) | | | |
| 7a | ssrna1_39 | CcaCas13b | caagcttgcatgcctgcaggtcgag | caagcttgca | GTTGGAACTGCT | ssRNA1 | 7a |
| | | | tagatGTTGGAACTGCTCTCATTTT | tgcctgcag | CTCATTTTGGAGG | | |
| | | | GGAGGGTAATCACAAC | gtcgagtag | GTAATCACAAC | | |
| | | | (SEQ ID. NO: 2217) | at (SEQ | (SEQ ID. | | |
| | | | | ID. NO: | NO: 2219) | | |
| | | | | 2218) | | | |
| 7a | ssrna1_40 | CcaCas13b | ccaagcttgcatgcctgcaggtcga | ccaagcttgc | GTTGGAACTGCT | ssRNA1 | 7a |
| | | | gtagaGTTGGAACTGCTCTCATTTT | atgcctgca | CTCATTTTGGAGG | | |
| | | | GGAGGGTAATCACAAC | ggtcgagta | GTAATCACAAC | | |
| | | | (SEQ ID. NO: 2220) | ga (SEQ | (SEQ ID. | | |
| | | | | ID. NO: | NO: 2222) | | |
| | | | | 2221) | | | |
| 7a | ssrna1_41 | CcaCas13b | gccaagcttgcatgcctgcaggtcg | gccaagctt | GTTGGAACTGCT | ssRNA1 | 7a |
| | | | agtagGTTGGAACTGCTCTCATTTT | gcatgcctg | CTCATTTTGGAGG | | |
| | | | GGAGGGTAATCACAAC | caggtcgag | GTAATCACAAC | | |
| | | | (SEQ ID. NO: 2223) | tag (SEQ | (SEQ ID. | | |
| | | | | ID. NO: | NO: 2225) | | |
| | | | | 2224) | | | |
| 7a | ssrna1_42 | CcaCas13b | cgccaagcttgcatgcctgcaggtc | cgccaagctt | GTTGGAACTGCT | ssRNA1 | 7a |
| | | | gagtaGTTGGAACTGCTCTCATTTT | gcatgcctg | CTCATTTTGGAGG | | |
| | | | GGAGGGTAATCACAAC | caggtcgag | GTAATCACAAC | | |
| | | | (SEQ ID. NO: 2226) | ta (SEQ | (SEQ ID. | | |
| | | | | ID. NO: | NO: 2228) | | |
| | | | | 2227) | | | |
| 7a | ssrna1_43 | CcaCas13b | tacgccaagcttgcatgcctgcagg | tacgccaag | GTTGGAACTGCT | ssRNA1 | 7a |
| | | | tcgagGTTGGAACTGCTCTCATTTT | cttgcatgcc | CTCATTTTGGAGG | | |
| | | | GGAGGGTAATCACAAC | tgcaggtcg | GTAATCACAAC | | |
| | | | (SEQ ID. NO: 2229) | ag (SEQ | (SEQ ID. | | |
| | | | | ID. NO: | NO: 2231) | | |
| | | | | 2230) | | | |
| 7a | ssrna1_44 | CcaCas13b | ttacgccaagcttgcatgcctgcag | ttacgccaag | GTTGGAACTGCT | ssRNA1 | 7a |
| | | | gtcgaGTTGGAACTGCTCTCATTTT | cttgcatgcc | CTCATTTTGGAGG | | |
| | | | GGAGGGTAATCACAAC | tgcaggtcg | GTAATCACAAC | | |
| | | | (SEQ ID. NO: 2232) | a (SEQ | (SEQ ID. | | |
| | | | | ID. NO: | NO: 2234) | | |
| | | | | 2233) | | | |
| 7a | ssrna1_45 | CcaCas13b | attacgccaagcttgcatgcctgca | attacgccaa | GTTGGAACTGCT | ssRNA1 | 7a |
| | | | ggtcgGTTGGAACTGCTCTCATTTT | gcttgcatgc | CTCATTTTGGAGG | | |
| | | | GGAGGGTAATCACAAC | ctgcaggtc | GTAATCACAAC | | |
| | | | (SEQ ID. NO: 2235) | g (SEQ | (SEQ ID. | | |
| | | | | ID. NO: | NO: 2237) | | |
| | | | | 2236) | | | |
| 7a | ssrna1_46 | CcaCas13b | gattacgccaagcttgcatgcctgc | gattacgcca | GTTGGAACTGCT | ssRNA1 | 7a |
| | | | aggtcGTTGGAACTGCTCTCATTTT | agcttgcatg | CTCATTTTGGAGG | | |
| | | | GGAGGGTAATCACAAC | cctgcaggt | GTAATCACAAC | | |
| | | | (SEQ ID. NO: 2238) | c (SEQ | (SEQ ID. | | |
| | | | | ID. NO: | NO: 2240) | | |
| | | | | 2239) | | | |
| 7a | ssrna1_47 | CcaCas13b | tgattacgccaagcttgcatgcctg | tgattacgcc | GTTGGAACTGCT | ssRNA1 | 7a |
| | | | caggtGTTGGAACTGCTCTCATTTT | aagcttgcat | CTCATTTTGGAGG | | |
| | | | GGAGGGTAATCACAAC | gcctgcagg | GTAATCACAAC | | |
| | | | (SEQ ID. NO: 2241) | t (SEQ | (SEQ ID. | | |
| | | | | ID. NO: | NO: 2243) | | |
| | | | | 2242) | | | |
| 7a | ssrna1_48 | CcaCas13b | atgattacgccaagcttgcatgcct | atgattacgc | GTTGGAACTGCT | ssRNA1 | 7a |
| | | | gcaggGTTGGAACTGCTCTCATTTT | caagcttgca | CTCATTTTGGAGG | | |
| | | | GGAGGGTAATCACAAC | tgcctgcag | GTAATCACAAC | | |
| | | | (SEQ ID. NO: 2244) | g (SEQ | (SEQ ID. | | |
| | | | | ID. NO: | NO: 2246) | | |
| | | | | 2245) | | | |
| 7a | ssrna1_49 | CcaCas13b | catgattacgccaagcttgcatgcc | catgattacg | GTTGGAACTGCT | ssRNA1 | 7a |
| | | | tgcagGTTGGAACTGCTCTCATTTT | ccaagcttgc | CTCATTTTGGAGG | | |
| | | | GGAGGGTAATCACAAC | atgcctgca | GTAATCACAAC | | |
| | | | (SEQ ID. NO: 2247) | g (SEQ | (SEQ ID. | | |
| | | | | ID. NO: | NO: 2249) | | |
| | | | | 2248) | | | |
| 7a | ssrna1_50 | CcaCas13b | accatgattacgccaagcttgcatg | accatgatta | GTTGGAACTGCT | ssRNA1 | 7a |
| | | | cctgcGTTGGAACTGCTCTCATTTT | cgccaagctt | CTCATTTTGGAGG | | |
| | | | GGAGGGTAATCACAAC | gcatgcctg | GTAATCACAAC | | |
| | | | (SEQ ID. NO: 2250) | c (SEQ | (SEQ ID. | | |
| | | | | ID. NO: | NO: 2252) | | |
| | | | | 2251) | | | |
| 7a | ssrna1_51 | CcaCas13b | gaccatgattacgccaagcttgcat | gaccatgatt | GTTGGAACTGCT | ssRNA1 | 7a |
| | | | gcctgGTTGGAACTGCTCTCATTTT | acgccaagc | CTCATTTTGGAGG | | |
| | | | GGAGGGTAATCACAAC | ttgcatgcct | GTAATCACAAC | | |
| | | | (SEQ ID. NO: 2253) | g (SEQ | (SEQ ID. | | |
| | | | | ID. NO: | NO: 2255) | | |
| | | | | 2254) | | | |
| 7a | ssrna1_52 | CcaCas13b | tgaccatgattacgccaagcttgca | tgaccatgat | GTTGGAACTGCT | ssRNA1 | 7a |
| | | | tgcctGTTGGAACTGCTCTCATTTT | tacgccaag | CTCATTTTGGAGG | | |
| | | | GGAGGGTAATCACAAC | cttgcatgcc | GTAATCACAAC | | |
| | | | (SEQ ID. NO: 2256) | t (SEQ | (SEQ ID. | | |
| | | | | ID. NO: | NO: 2258) | | |
| | | | | 2257) | | | |
| 7a | ssrna1_53 | CcaCas13b | atgaccatgattacgccaagcttgc | atgaccatga | GTTGGAACTGCT | ssRNA1 | 7a |
| | | | atgccGTTGGAACTGCTCTCATTTT | ttacgccaag | CTCATTTTGGAGG | | |
| | | | GGAGGGTAATCACAAC | cttgcatgcc | GTAATCACAAC | | |
| | | | (SEQ ID. NO: 2259) | (SEQ ID. | (SEQ ID. | | |
| | | | | NO: | NO: 2261) | | |
| | | | | 2260) | | | |
| 7a | ssrna1_54 | CcaCas13b | ctatgaccatgattacgccaagctt | ctatgaccat | GTTGGAACTGCT | ssRNA1 | 7a |
| | | | gcatgGTTGGAACTGCTCTCATTTT | gattacgcca | CTCATTTTGGAGG | | |
| | | | GGAGGGTAATCACAAC | agcttgcatg | GTAATCACAAC | | |
| | | | (SEQ ID. NO: 2262) | (SEQ ID. | (SEQ ID. | | |
| | | | | NO: | NO: 2264) | | |
| | | | | 2263) | | | |
| 7a | ssrna1_55 | CcaCas13b | gctatgaccatgattacgccaagct | gctatgacca | GTTGGAACTGCT | ssRNA1 | 7a |
| | | | tgcatGTTGGAACTGCTCTCATTTT | tgattacgcc | CTCATTTTGGAGG | | |
| | | | GGAGGGTAATCACAAC | aagcttgcat | GTAATCACAAC | | |
| | | | (SEQ ID. NO: 2265) | (SEQ ID. | (SEQ ID. | | |
| | | | | NO: | NO: 2267) | | |
| | | | | 2266) | | | |
| 7a | ssrna1_56 | CcaCas13b | acagctatgaccatgattacgccaa | acagctatga | GTTGGAACTGCT | ssRNA1 | 7a |
| | | | gcttgGTTGGAACTGCTCTCATTTT | ccatgattac | CTCATTTTGGAGG | | |
| | | | GGAGGGTAATCACAAC | gccaagctt | GTAATCACAAC | | |
| | | | (SEQ ID. NO: 2268) | g (SEQ | (SEQ ID. | | |
| | | | | ID. NO: | NO: 2270) | | |
| | | | | 2269) | | | |
| 7a | ssrna1_57 | CcaCas13b | aacagctatgaccatgattacgcca | aacagctatg | GTTGGAACTGCT | ssRNA1 | 7a |
| | | | agcttGTTGGAACTGCTCTCATTTT | accatgatta | CTCATTTTGGAGG | | |
| | | | GGAGGGTAATCACAAC | cgccaagctt | GTAATCACAAC | | |
| | | | (SEQ ID. NO: 2271) | (SEQ ID. | (SEQ ID. | | |
| | | | | NO: | NO: 2273) | | |
| | | | | 2272) | | | |
| 7a | ssrna1_58 | CcaCas13b | aaacagctatgaccatgattacgcc | aaacagctat | GTTGGAACTGCT | ssRNA1 | 7a |
| | | | aagctGTTGGAACTGCTCTCATTTT | gaccatgatt | CTCATTTTGGAGG | | |
| | | | GGAGGGTAATCACAAC | acgccaagc | GTAATCACAAC | | |
| | | | (SEQ ID. NO: 2274) | t (SEQ | (SEQ ID. | | |
| | | | | ID. NO: | NO: 2276) | | |
| | | | | 2275) | | | |
| 7a | ssrna1_59 | CcaCas13b | gaaacagctatgaccatgattacgc | gaaacagct | GTTGGAACTGCT | ssRNA1 | 7a |
| | | | caagcGTTGGAACTGCTCTCATTTT | atgaccatga | CTCATTTTGGAGG | | |
| | | | GGAGGGTAATCACAAC | ttacgccaag | GTAATCACAAC | | |
| | | | (SEQ ID. NO: 2277) | c (SEQ | (SEQ ID. | | |
| | | | | ID. NO: | NO: 2279) | | |
| | | | | 2278) | | | |
| 7a | ssrna1_60 | CcaCas13b | caggaaacagctatgaccatgatta | caggaaaca | GTTGGAACTGCT | ssRNA1 | 7a |
| | | | cgccaGTTGGAACTGCTCTCATTTT | gctatgacca | CTCATTTTGGAGG | | |
| | | | GGAGGGTAATCACAAC | tgattacgcc | GTAATCACAAC | | |
| | | | (SEQ ID. NO: 2280) | a (SEQ | (SEQ ID. | | |
| | | | | ID. NO: | NO: 2282) | | |
| | | | | 2281) | | | |
| 7a | ssrna1_61 | CcaCas13b | acaggaaacagctatgaccatgatt | acaggaaac | GTTGGAACTGCT | ssRNA1 | 7a |
| | | | acgccGTTGGAACTGCTCTCATTTT | agctatgacc | CTCATTTTGGAGG | | |
| | | | GGAGGGTAATCACAAC | atgattacgc | GTAATCACAAC | | |
| | | | (SEQ ID. NO: 2283) | c (SEQ | (SEQ ID. | | |
| | | | | ID. NO: | NO: 2285) | | |
| | | | | 2284) | | | |
| 7a | ssrna1_62 | CcaCas13b | cacaggaaacagctatgaccatgat | cacaggaaa | GTTGGAACTGCT | ssRNA1 | 7a |
| | | | tacgcGTTGGAACTGCTCTCATTTT | cagctatgac | CTCATTTTGGAGG | | |
| | | | GGAGGGTAATCACAAC | catgattacg | GTAATCACAAC | | |
| | | | (SEQ ID. NO: 2286) | c (SEQ | (SEQ ID. | | |
| | | | | ID. NO: | NO: 2288) | | |
| | | | | 2287) | | | |
| 7a | ssrna1_63 | CcaCas13b | taaacacaggaaacagctatgacca | taaacacag | GTTGGAACTGCT | ssRNA1 | 7a |
| | | | tgattGTTGGAACTGCTCTCATTTT | gaaacagct | CTCATTTTGGAGG | | |
| | | | GGAGGGTAATCACAAC | atgaccatga | GTAATCACAAC | | |
| | | | (SEQ ID. NO: 2289) | tt (SEQ | (SEQ ID. | | |
| | | | | ID. NO: | NO: 2291) | | |
| | | | | 2290) | | | |
| 7a | ssrna1_64 | CcaCas13b | gataaacacaggaaacagctatgac | gataaacac | GTTGGAACTGCT | ssRNA1 | 7a |
| | | | catgaGTTGGAACTGCTCTCATTTT | aggaaacag | CTCATTTTGGAGG | | |
| | | | GGAGGGTAATCACAAC | ctatgaccat | GTAATCACAAC | | |
| | | | (SEQ ID. NO: 2292) | ga (SEQ | (SEQ ID. | | |
| | | | | ID. NO: | NO: 2294) | | |
| | | | | 2293) | | | |
| 7a | ssrna1_65 | CcaCas13b | ggataaacacaggaaacagctatga | ggataaaca | GTTGGAACTGCT | ssRNA1 | 7a |
| | | | ccatgGTTGGAACTGCTCTCATTTT | caggaaaca | CTCATTTTGGAGG | | |
| | | | GGAGGGTAATCACAAC | gctatgacca | GTAATCACAAC | | |
| | | | (SEQ ID. NO: 2295) | tg (SEQ | (SEQ ID. | | |
| | | | | ID. NO: | NO: 2297) | | |
| | | | | 2296) | | | |
| 7a | ssrna1_66 | CcaCas13b | cggataaacacaggaaacagctatg | cggataaac | GTTGGAACTGCT | ssRNA1 | 7a |
| | | | accatGTTGGAACTGCTCTCATTTT | acaggaaac | CTCATTTTGGAGG | | |
| | | | GGAGGGTAATCACAAC | agctatgacc | GTAATCACAAC | | |
| | | | (SEQ ID. NO: 2298) | at (SEQ | (SEQ ID. | | |
| | | | | ID. NO: | NO: 2300) | | |
| | | | | 2299) | | | |
| 7a | ssrna1_67 | CcaCas13b | gcggataaacacaggaaacagctat | gcggataaa | GTTGGAACTGCT | ssRNA1 | 7a |
| | | | gaccaGTTGGAACTGCTCTCATTTT | cacaggaaa | CTCATTTTGGAGG | | |
| | | | GGAGGGTAATCACAAC | cagctatgac | GTAATCACAAC | | |
| | | | (SEQ ID. NO: 2301) | ca (SEQ | (SEQ ID. | | |
| | | | | ID. NO: | NO: 2303) | | |
| | | | | 2302) | | | |
| 7a | ssrna1_68 | CcaCas13b | agcggataaacacaggaaacagcta | agcggataa | GTTGGAACTGCT | ssRNA1 | 7a |
| | | | tgaccGTTGGAACTGCTCTCATTTT | acacaggaa | CTCATTTTGGAGG | | |
| | | | GGAGGGTAATCACAAC | acagctatga | GTAATCACAAC | | |
| | | | (SEQ ID. NO: 2304) | cc (SEQ | (SEQ ID. | | |
| | | | | ID. NO: | NO: 2306) | | |
| | | | | 2305) | | | |
| 7a | ssrna1_69 | CcaCas13b | gagcggataaacacaggaaacagct | gageggata | GTTGGAACTGCT | ssRNA1 | 7a |
| | | | atgacGTTGGAACTGCTCTCATTT | aacacagga | CTCATTTTGGAGG | | |
| | | | TGGAGGGTAATCACAAC | aacagctatg | GTAATCACAAC | | |
| | | | (SEQ ID. NO: 2307) | ac (SEQ | (SEQ ID. | | |
| | | | | ID. NO: | NO: 2309) | | |
| | | | | 2308) | | | |
| 7a | ssrna1_70 | CcaCas13b | tgagcggataaacacaggaaacagc | tgagcggat | GTTGGAACTGCT | ssRNA1 | 7a |
| | | | tatgaGTTGGAACTGCTCTCATTTT | aaacacagg | CTCATTTTGGAGG | | |
| | | | GGAGGGTAATCACAAC | aaacagctat | GTAATCACAAC | | |
| | | | (SEQ ID. NO: 2310) | ga (SEQ | (SEQ ID. | | |
| | | | | ID. NO: | NO: 2312) | | |
| | | | | 2311) | | | |
| 7a | ssrna1_71 | CcaCas13b | tgtgagcggataaacacaggaaaca | tgtgagcgg | GTTGGAACTGCT | ssRNA1 | 7a |
| | | | gctatGTTGGAACTGCTCTCATTTT | ataaacaca | CTCATTTTGGAGG | | |
| | | | GGAGGGTAATCACAAC | ggaaacagc | GTAATCACAAC | | |
| | | | (SEQ ID. NO: 2313) | tat (SEQ | (SEQ ID. | | |
| | | | | ID. NO: | NO: 2315) | | |
| | | | | 2314) | | | |
| 7a | ssrna1_72 | CcaCas13b | attgtgagcggataaacacaggaaa | attgtgagcg | GTTGGAACTGCT | ssRNA1 | 7a |
| | | | cagctGTTGGAACTGCTCTCATTTT | gataaacac | CTCATTTTGGAGG | | |
| | | | GGAGGGTAATCACAAC | aggaaacag | GTAATCACAAC | | |
| | | | (SEQ ID. NO: 2316) | ct (SEQ | (SEQ ID. | | |
| | | | | ID. NO: | NO: 2318) | | |
| | | | | 2317) | | | |
| 7a | ssrna1_73 | CcaCas13b | aattgtgagcggataaacacaggaa | aattgtgagc | GTTGGAACTGCT | ssRNA1 | 7a |
| | | | cagcGTTGGAACTGCTCTCATTTT | ggataaaca | CTCATTTTGGAGG | | |
| | | | GGAGGGTAATCACAAC | caggaaaca | GTAATCACAAC | | |
| | | | (SEQ ID. NO: 2319) | gc (SEQ | (SEQ ID. | | |
| | | | | ID. NO: | NO: 2321) | | |
| | | | | 2320) | | | |
| 7a | ssrna1_74 | CcaCas13b | gaattgtgagcggataaacacagga | gaattgtgag | GTTGGAACTGCT | ssRNA1 | 7a |
| | | | aacagGTTGGAACTGCTCTCATTTT | cggataaac | CTCATTTTGGAGG | | |
| | | | GGAGGGTAATCACAAC | acaggaaac | GTAATCACAAC | | |
| | | | (SEQ ID. NO: 2322) | ag (SEQ | (SEQ ID. | | |
| | | | | ID. NO: | NO: 2324) | | |
| | | | | 2323) | | | |
| 7a | ssrna1_75 | CcaCas13b | gtggaattgtgagcggataaacaca | gtggaattgt | GTTGGAACTGCT | ssRNA1 | 7a |
| | | | ggaaaGTTGGAACTGCTCTCATTTT | gagcggata | CTCATTTTGGAGG | | |
| | | | GGAGGGTAATCACAAC | aacacagga | GTAATCACAAC | | |
| | | | (SEQ ID. NO: 2325) | aa (SEQ | (SEQ ID. | | |
| | | | | ID. NO: | NO: 2327) | | |
| | | | | 2326) | | | |
| 7a | ssrna1_76 | CcaCas13b | tgtggaattgtgagcggataaacac | tgtggaattg | GTTGGAACTGCT | ssRNA1 | 7a |
| | | | aggaaGTTGGAACTGCTCTCATTTT | tgagcggat | CTCATTTTGGAGG | | |
| | | | GGAGGGTAATCACAAC | aaacacagg | GTAATCACAAC | | |
| | | | (SEQ ID. NO: 2328) | aa (SEQ | (SEQ ID. | | |
| | | | | ID. NO: | NO: 2330) | | |
| | | | | 2329) | | | |
| 7a | ssrna1_77 | CcaCas13b | gtgtggaattgtgagcggataaaca | gtgtggaatt | GTTGGAACTGCT | ssRNA1 | 7a |
| | | | caggaGTTGGAACTGCTCTCATTTT | gtgagcgga | CTCATTTTGGAGG | | |
| | | | GGAGGGTAATCACAAC | taaacacag | GTAATCACAAC | | |
| | | | (SEQ ID. NO: 2331) | ga (SEQ | (SEQ ID. | | |
| | | | | ID. NO: | NO: 2333) | | |
| | | | | 2332) | | | |
| 7a | ssrna1_78 | CcaCas13b | tgtgtggaattgtgagcggataaac | tgtgtggaat | GTTGGAACTGCT | ssRNA1 | 7a |
| | | | acaggGTTGGAACTGCTCTCATTTT | tgtgagcgg | CTCATTTTGGAGG | | |
| | | | GGAGGGTAATCACAAC | ataaacaca | GTAATCACAAC | | |
| | | | (SEQ ID. NO: 2334) | gg (SEQ | (SEQ ID. | | |
| | | | | ID. NO: | NO: 2336) | | |
| | | | | 2335) | | | |
| 7a | ssrna1_79 | CcaCas13b | gttgtgtggaattgtgagcggataa | gttgtgtgga | GTTGGAACTGCT | ssRNA1 | 7a |
| | | | acacaGTTGGAACTGCTCTCATTTT | attgtgagcg | CTCATTTTGGAGG | | |
| | | | GGAGGGTAATCACAAC | gataaacac | GTAATCACAAC | | |
| | | | (SEQ ID. NO: 2337) | a (SEQ | (SEQ ID. | | |
| | | | | ID. NO: | NO: 2339) | | |
| | | | | 2338) | | | |
| 7a | ssrna1_80 | CcaCas13b | tgttgtgtggaattgtgagcggata | tgttgtgtgg | GTTGGAACTGCT | ssRNA1 | 7a |
| | | | aacacGTTGGAACTGCTCTCATTTT | aattgtgagc | CTCATTTTGGAGG | | |
| | | | GGAGGGTAATCACAAC | ggataaaca | GTAATCACAAC | | |
| | | | (SEQ ID. NO: 2340) | c (SEQ | (SEQ ID. | | |
| | | | | ID. NO: | NO: 2342) | | |
| | | | | 2341) | | | |
| 7a | ssrna1_81 | CcaCas13b | atgttgtgtggaattgtgagcggat | atgttgtgtg | GTTGGAACTGCT | ssRNA1 | 7a |
| | | | aaacaGTTGGAACTGCTCTCATTTT | gaattgtgag | CTCATTTTGGAGG | | |
| | | | GGAGGGTAATCACAAC | cggataaac | GTAATCACAAC | | |
| | | | (SEQ ID. NO: 2343) | a (SEQ | (SEQ ID. | | |
| | | | | ID. NO: | NO: 2345) | | |
| | | | | 2344) | | | |
| 7a | ssrna1_82 | CcaCas13b | gtatgttgtgtggaattgtgagcgg | gtatgttgtgt | GTTGGAACTGCT | ssRNA1 | 7a |
| | | | ataaaGTTGGAACTGCTCTCATTTT | ggaattgtga | CTCATTTTGGAGG | | |
| | | | GGAGGGTAATCACAAC | gcggataaa | GTAATCACAAC | | |
| | | | (SEQ ID. NO: 2346) | (SEQ ID. | (SEQ ID. | | |
| | | | | NO: | NO: 2348) | | |
| | | | | 2347) | | | |
| 7a | ssrna1_83 | CcaCas13b | cgtatgttgtgtggaattgtgagcg | cgtatgttgt | GTTGGAACTGCT | ssRNA1 | 7a |
| | | | gataaGTTGGAACTGCTCTCATTTT | gtggaattgt | CTCATTTTGGAGG | | |
| | | | GGAGGGTAATCACAAC | gagcggata | GTAATCACAAC | | |
| | | | (SEQ ID. NO: 2349) | a (SEQ | (SEQ ID. | | |
| | | | | ID. NO: | NO: 2351) | | |
| | | | | 2350) | | | |
| 7a | ssrna1_84 | CcaCas13b | tcgtatgttgtgtggaattgtgagc | tcgtatgttgt | GTTGGAACTGCT | ssRNA1 | 7a |
| | | | ggataGTTGGAACTGCTCTCATTTT | gtggaattgt | CTCATTTTGGAGG | | |
| | | | GGAGGGTAATCACAAC | gagcggata | GTAATCACAAC | | |
| | | | (SEQ ID. NO: 2352) | (SEQ ID. | (SEQ ID. | | |
| | | | | NO: | NO: 2354) | | |
| | | | | 2353) | | | |
| 7a | ssrna1_85 | CcaCas13b | gctcgtatgttgtgtggaattgtga | gctcgtatgtt | GTTGGAACTGCT | ssRNA1 | 7a |
| | | | gcggaGTTGGAACTGCTCTCATTTT | gtgtggaatt | CTCATTTTGGAGG | | |
| | | | GGAGGGTAATCACAAC | gtgagcgga | GTAATCACAAC | | |
| | | | (SEQ ID. NO: 2355) | (SEQ ID. | (SEQ ID. | | |
| | | | | NO: | NO: 2357) | | |
| | | | | 2356) | | | |
| 7a | ssrna1_86 | CcaCas13b | ggctcgtatgttgtgtggaattgtg | ggctcgtatg | GTTGGAACTGCT | ssRNA1 | 7a |
| | | | agcggGTTGGAACTGCTCTCATTTT | ttgtgtggaa | CTCATTTTGGAGG | | |
| | | | GGAGGGTAATCACAAC | ttgtgagcgg | GTAATCACAAC | | |
| | | | (SEQ ID. NO: 2358) | (SEQ ID. | (SEQ ID. | | |
| | | | | NO: | NO: 2360) | | |
| | | | | 2359) | | | |
| 7a | ssrna1_87 | CcaCas13b | ccggctcgtatgttgtgtggaattg | ccggctcgt | GTTGGAACTGCT | ssRNA1 | 7a |
| | | | tgagcGTTGGAACTGCTCTCATTTT | atgttgtgtg | CTCATTTTGGAGG | | |
| | | | GGAGGGTAATCACAAC | gaattgtgag | GTAATCACAAC | | |
| | | | (SEQ ID. NO: 2361) | c (SEQ | (SEQ ID. | | |
| | | | | ID. NO: | NO: 2363) | | |
| | | | | 2362) | | | |
| 7a | ssrna1_88 | CcaCas13b | tccggctcgtatgttgtgtggaatt | tccggctcgt | GTTGGAACTGCT | ssRNA1 | 7a |
| | | | gtgagGTTGGAACTGCTCTCATTTT | atgttgtgtg | CTCATTTTGGAGG | | |
| | | | GGAGGGTAATCACAAC | gaattgtgag | GTAATCACAAC | | |
| | | | (SEQ ID. NO: 2364) | (SEQ ID. | (SEQ ID. | | |
| | | | | NO: | NO: 2366) | | |
| | | | | 2365) | | | |
| 7a | ssrna1_89 | CcaCas13b | ttccggctcgtatgttgtgtggaat | ttccggctcg | GTTGGAACTGCT | ssRNA1 | 7a |
| | | | tgtgaGTTGGAACTGCTCTCATTTT | tatgttgtgtg | CTCATTTTGGAGG | | |
| | | | GGAGGGTAATCACAAC | gaattgtga | GTAATCACAAC | | |
| | | | (SEQ ID. NO: 2367) | (SEQ ID. | (SEQ ID. | | |
| | | | | NO: | NO: 2369) | | |
| | | | | 2368) | | | |
| 7a | ssrna1_90 | CcaCas13b | gcttccggctcgtatgttgtgtgga | gcttccggct | GTTGGAACTGCT | ssRNA1 | 7a |
| | | | attgtGTTGGAACTGCTCTCATTTT | cgtatgttgt | CTCATTTTGGAGG | | |
| | | | GGAGGGTAATCACAAC | gtggaattgt | GTAATCACAAC | | |
| | | | (SEQ ID. NO: 2370) | (SEQ ID. | (SEQ ID. | | |
| | | | | NO: | NO: 2372) | | |
| | | | | 2371) | | | |
| 7a | ssrna1_91 | CcaCas13b | tgcttccggctcgtatgttgtgtgg | tgcttccggc | GTTGGAACTGCT | ssRNA1 | 7a |
| | | | aattgGTTGGAACTGCTCTCATTTT | tcgtatgttgt | CTCATTTTGGAGG | | |
| | | | GGAGGGTAATCACAAC | gtggaattg | GTAATCACAAC | | |
| | | | (SEQ ID. NO: 2373) | (SEQ ID. | (SEQ ID. | | |
| | | | | NO: | NO: 2375) | | |
| | | | | 2374) | | | |
| 7a | ssrna1_92 | CcaCas13b | atgcttccggctcgtatgttgtgtg | atgcttccgg | GTTGGAACTGCT | ssRNA1 | 7a |
| | | | gaattGTTGGAACTGCTCTCATTTT | ctcgtatgttg | CTCATTTTGGAGG | | |
| | | | GGAGGGTAATCACAAC | tgtggaatt | GTAATCACAAC | | |
| | | | (SEQ ID. NO: 2376) | (SEQ ID. | (SEQ ID. | | |
| | | | | NO: | NO: 2378) | | |
| | | | | 2377) | | | |
| 7a | ssrna1_93 | CcaCas13b | ttatgcttccggctcgtatgttgtg | ttatgcttccg | GTTGGAACTGCT | ssRNA1 | 7a |
| | | | tggaaGTTGGAACTGCTCTCATTTT | gctcgtatgtt | CTCATTTTGGAGG | | |
| | | | GGAGGGTAATCACAAC | gtgtggaa | GTAATCACAAC | | |
| | | | (SEQ ID. NO: 2379) | (SEQ ID. | (SEQ ID. | | |
| | | | | NO: | NO: 2381) | | |
| | | | | 2380) | | | |
| 7a | ssrna1_94 | CcaCas13b | tttatgcttccggctcgtatgttgt | tttatgcttcc | GTTGGAACTGCT | ssRNA1 | 7a |
| | | | gtggaGTTGGAACTGCTCTCATTTT | ggctcgtatg | CTCATTTTGGAGG | | |
| | | | GGAGGGTAATCACAAC | ttgtgtgga | GTAATCACAAC | | |
| | | | (SEQ ID. NO: 2382) | (SEQ ID. | (SEQ ID. | | |
| | | | | NO: | NO: 2384) | | |
| | | | | 2383) | | | |
| 1b | ebola_0 | CcaCas13b | aactgtgaaagacaactcttcactg | aactgtgaaa | GTTGGAACTGCT | Ebola | 1b |
| | | | cgaatGTTGGAACTGCTCTCATTT | gacaactctt | CTCATTTTGGAGG | ssRNA | |
| | | | TGGAGGGTAATCACAAC | cactgcgaat | GTAATCACAAC | | |
| | | | (SEQ ID. NO: 2385) | (SEQ ID. | (SEQ ID. | | |
| | | | | NO: | NO: 2387) | | |
| | | | | 2386) | | | |
| 1b | ebola_1 | CcaCas13b | caactgtgaaagacaactcttcact | caactgtgaa | GTTGGAACTGCT | Ebola | 1b |
| | | | gcgaaGTTGGAACTGCTCTCATTTT | agacaactct | CTCATTTTGGAGG | ssRNA | |
| | | | GGAGGGTAATCACAAC | tcactgcgaa | GTAATCACAAC | | |
| | | | (SEQ ID. NO: 2388) | (SEQ ID. | (SEQ ID. | | |
| | | | | NO: | NO: 2390) | | |
| | | | | 2389) | | | |
| 1b | ebola_2 | CcaCas13b | acaactgtgaaagacaactcttcac | acaactgtga | GTTGGAACTGCT | Ebola | 1b |
| | | | tgcgaGTTGGAACTGCTCTCATTTT | aagacaact | CTCATTTTGGAGG | ssRNA | |
| | | | GGAGGGTAATCACAAC | cttcactgcg | GTAATCACAAC | | |
| | | | (SEQ ID. NO: 2391) | a (SEQ | (SEQ ID. | | |
| | | | | ID. NO: | NO: 2393) | | |
| | | | | 2392) | | | |
| 1b | ebola_3 | CcaCas13b | atacaactgtgaaagacaactcttc | atacaactgt | GTTGGAACTGCT | Ebola | 1b |
| | | | actgcGTTGGAACTGCTCTCATTTT | gaaagacaa | CTCATTTTGGAGG | ssRNA | |
| | | | GGAGGGTAATCACAAC | ctcttcactg | GTAATCACAAC | | |
| | | | (SEQ ID. NO: 2394) | c (SEQ | (SEQ ID. | | |
| | | | | ID. NO: | NO: 2396) | | |
| | | | | 2395) | | | |
| 1b | ebola_4 | CcaCas13b | gatacaactgtgaaagacaactctt | gatacaactg | GTTGGAACTGCT | Ebola | 1b |
| | | | cactgGTTGGAACTGCTCTCATTTT | tgaaagaca | CTCATTTTGGAGG | ssRNA | |
| | | | GGAGGGTAATCACAAC | actcttcact | GTAATCACAAC | | |
| | | | (SEQ ID. NO: 2397) | g (SEQ | (SEQ ID. | | |
| | | | | ID. NO: | NO: 2399) | | |
| | | | | 2398) | | | |
| 1b | ebola_5 | CcaCas13b | ttgatacaactgtgaaagacaactc | ttgatacaac | GTTGGAACTGCT | Ebola | 1b |
| | | | ttcacGTTGGAACTGCTCTCATTTT | tgtgaaaga | CTCATTTTGGAGG | ssRNA | |
| | | | GGAGGGTAATCACAAC | caactcttca | GTAATCACAAC | | |
| | | | (SEQ ID. NO: 2400) | c (SEQ | (SEQ ID. | | |
| | | | | ID. NO: | NO: 2402) | | |
| | | | | 2401) | | | |
| 1b | ebola_6 | CcaCas13b | tttgatacaactgtgaaagacaact | tttgatacaa | GTTGGAACTGCT | Ebola | 1b |
| | | | cttcaGTTGGAACTGCTCTCATTTT | ctgtgaaag | CTCATTTTGGAGG | ssRNA | |
| | | | GGAGGGTAATCACAAC | acaactcttc | GTAATCACAAC | | |
| | | | (SEQ ID. NO: 2403) | a (SEQ | (SEQ ID. | | |
| | | | | ID. NO: | NO: 2405) | | |
| | | | | 2404) | | | |
| 1b | ebola_7 | CcaCas13b | cgtttgatacaactgtgaaagacaa | cgtttgatac | GTTGGAACTGCT | Ebola | 1b |
| | | | ctcttGTTGGAACTGCTCTCATTTT | aactgtgaaa | CTCATTTTGGAGG | ssRNA | |
| | | | GGAGGGTAATCACAAC | gacaactctt | GTAATCACAAC | | |
| | | | (SEQ ID. NO: 2406) | (SEQ ID. | (SEQ ID. | | |
| | | | | NO: | NO: 2408) | | |
| | | | | 2407) | | | |
| 1b | ebola_8 | CcaCas13b | ccgtttgatacaactgtgaaagaca | ccgtttgata | GTTGGAACTGCT | Ebola | 1b |
| | | | actctGTTGGAACTGCTCTCATTTT | caactgtgaa | CTCATTTTGGAGG | ssRNA | |
| | | | GGAGGGTAATCACAAC | agacaactct | GTAATCACAAC | | |
| | | | (SEQ ID. NO: 2409) | (SEQ ID. | (SEQ ID. | | |
| | | | | NO: | NO: 2411) | | |
| | | | | 2410) | | | |
| 1b | ebola_9 | CcaCas13b | ctccgtttgatacaactgtgaaaga | ctccgtttgat | GTTGGAACTGCT | Ebola | 1b |
| | | | caactGTTGGAACTGCTCTCATTTT | acaactgtga | CTCATTTTGGAGG | ssRNA | |
| | | | GGAGGGTAATCACAAC | aagacaact | GTAATCACAAC | | |
| | | | (SEQ ID. NO: 2412) | (SEQ ID. | (SEQ ID. | | |
| | | | | NO: | NO: 2414) | | |
| | | | | 2413) | | | |
| 1b | ebola_10 | CcaCas13b | gctccgtttgatacaactgtgaaag | gctccgtttg | GTTGGAACTGCT | Ebola | 1b |
| | | | acaacGTTGGAACTGCTCTCATTTT | atacaactgt | CTCATTTTGGAGG | ssRNA | |
| | | | GGAGGGTAATCACAAC | gaaagacaa | GTAATCACAAC | | |
| | | | (SEQ ID. NO: 2415) | c (SEQ | (SEQ ID. | | |
| | | | | ID. NO: | NO: 2417) | | |
| | | | | 2416) | | | |
| 1b | ebola_11 | CcaCas13b | tggctccgtttgatacaactgtgaa | tggctccgttt | GTTGGAACTGCT | Ebola | 1b |
| | | | agacaGTTGGAACTGCTCTCATTTT | gatacaactg | CTCATTTTGGAGG | ssRNA | |
| | | | GGAGGGTAATCACAAC | tgaaagaca | GTAATCACAAC | | |
| | | | (SEQ ID. NO: 2418) | (SEQ ID. | (SEQ ID. | | |
| | | | | NO: | NO: 2420) | | |
| | | | | 2419) | | | |
| 1b | ebola_12 | CcaCas13b | ttggctccgtttgatacaactgtga | ttggctccgtt | GTTGGAACTGCT | Ebola | 1b |
| | | | aagacGTTGGAACTGCTCTCATTTT | tgatacaact | CTCATTTTGGAGG | ssRNA | |
| | | | GGAGGGTAATCACAAC | gtgaaagac | GTAATCACAAC | | |
| | | | (SEQ ID. NO: 2421) | (SEQ ID. | (SEQ ID. | | |
| | | | | NO: | NO: 2423) | | |
| | | | | 2422) | | | |
| 1b | ebola_13 | CcaCas13b | tttggctccgtttgatacaactgtg | tttggctccgt | GTTGGAACTGCT | Ebola | 1b |
| | | | aaagaGTTGGAACTGCTCTCATTTT | ttgatacaac | CTCATTTTGGAGG | ssRNA | |
| | | | GGAGGGTAATCACAAC | tgtgaaaga | GTAATCACAAC | | |
| | | | (SEQ ID. NO: 2424) | (SEQ ID. | (SEQ ID. | | |
| | | | | NO: | NO: 2426) | | |
| | | | | 2425) | | | |
| 1b | ebola_14 | CcaCas13b | tttttggctccgtttgatacaactg | tttttggctcc | GTTGGAACTGCT | Ebola | 1b |
| | | | tgaaaGTTGGAACTGCTCTCATTTT | gtttgataca | CTCATTTTGGAGG | ssRNA | |
| | | | GGAGGGTAATCACAAC | actgtgaaa | GTAATCACAAC | | |
| | | | (SEQ ID. NO: 2427) | (SEQ ID. | (SEQ ID. | | |
| | | | | NO: | NO: 2429) | | |
| | | | | 2428) | | | |
| 1b | ebola_15 | CcaCas13b | gatgtttttggctccgtttgataca | gatgtttttgg | GTTGGAACTGCT | Ebola | 1b |
| | | | actgtGTTGGAACTGCTCTCATTTT | ctccgtttgat | CTCATTTTGGAGG | ssRNA | |
| | | | GGAGGGTAATCACAAC | acaactgt | GTAATCACAAC | | |
| | | | (SEQ ID. NO: 2430) | (SEQ ID. | (SEQ ID. | | |
| | | | | NO: | NO: 2432) | | |
| | | | | 2431) | | | |
| 1b | ebola_16 | CcaCas13b | tgatgtttttggctccgtttgatac | tgatgtttttg | GTTGGAACTGCT | Ebola | 1b |
| | | | aactgGTTGGAACTGCTCTCATTTT | gctccgtttg | CTCATTTTGGAGG | ssRNA | |
| | | | GGAGGGTAATCACAAC | atacaactg | GTAATCACAAC | | |
| | | | (SEQ ID. NO: 2433) | (SEQ ID. | (SEQ ID. | | |
| | | | | NO: | NO: 2435) | | |
| | | | | 2434) | | | |
| 1b | ebola_17 | CcaCas13b | ctgatgtttttggctccgtttgata | ctgatgttttt | GTTGGAACTGCT | Ebola | 1b |
| | | | caactGTTGGAACTGCTCTCATTTT | ggctccgttt | CTCATTTTGGAGG | ssRNA | |
| | | | GGAGGGTAATCACAAC | gatacaact | GTAATCACAAC | | |
| | | | (SEQ ID. NO: 2436) | (SEQ ID. | (SEQ ID. | | |
| | | | | NO: | NO: 2438) | | |
| | | | | 2437) | | | |
| 1b | ebola_18 | CcaCas13b | actgatgtttttggctccgtttgat | actgatgtttt | GTTGGAACTGCT | Ebola | 1b |
| | | | acaacGTTGGAACTGCTCTCATTTT | tggctccgttt | CTCATTTTGGAGG | ssRNA | |
| | | | GGAGGGTAATCACAAC | gatacaac | GTAATCACAAC | | |
| | | | (SEQ ID. NO: 2439) | (SEQ ID. | (SEQ ID. | | |
| | | | | NO: | NO: 2441) | | |
| | | | | 2440) | | | |
| 1b | ebola_19 | CcaCas13b | gaccactgatgtttttggctccgtt | gaccactgat | GTTGGAACTGCT | Ebola | 1b |
| | | | tgataGTTGGAACTGCTCTCATTTT | gtttttggctc | CTCATTTTGGAGG | ssRNA | |
| | | | GGAGGGTAATCACAAC | cgtttgata | GTAATCACAAC | | |
| | | | (SEQ ID. NO: 2442) | (SEQ ID. | (SEQ ID. | | |
| | | | | NO: | NO: 2444) | | |
| | | | | 2443) | | | |
| 1b | ebola_20 | CcaCas13b | tgaccactgatgtttttggctccgt | tgaccactga | GTTGGAACTGCT | Ebola | 1b |
| | | | ttgatGTTGGAACTGCTCTCATTTT | tgtttttggct | CTCATTTTGGAGG | ssRNA | |
| | | | GGAGGGTAATCACAAC | ccgtttgat | GTAATCACAAC | | |
| | | | (SEQ ID. NO: 2445) | (SEQ ID. | (SEQ ID. | | |
| | | | | NO: | NO: 2447) | | |
| | | | | 2446) | | | |
| 1b | ebola_21 | CcaCas13b | ctgaccactgatgtttttggctccg | ctgaccactg | GTTGGAACTGCT | Ebola | 1b |
| | | | tttgaGTTGGAACTGCTCTCATTTT | atgtttttggc | CTCATTTTGGAGG | ssRNA | |
| | | | GGAGGGTAATCACAAC | tccgtttga | GTAATCACAAC | | |
| | | | (SEQ ID. NO: 2448) | (SEQ ID. | (SEQ ID. | | |
| | | | | NO: | NO: 2450) | | |
| | | | | 2449) | | | |
| 1b | ebola_22 | CcaCas13b | ctctgaccactgatgtttttggctc | ctctgaccac | GTTGGAACTGCT | Ebola | 1b |
| | | | cgtttGTTGGAACTGCTCTCATTTT | tgatgtttttg | CTCATTTTGGAGG | ssRNA | |
| | | | GGAGGGTAATCACAAC | gctccgttt | GTAATCACAAC | | |
| | | | (SEQ ID. NO: 2451) | (SEQ ID. | (SEQ ID. | | |
| | | | | NO: | NO: 2453) | | |
| | | | | 2452) | | | |
| 1b | ebola_23 | CcaCas13b | actctgaccactgatgtttttggct | actctgacca | GTTGGAACTGCT | Ebola | 1b |
| | | | ccgttGTTGGAACTGCTCTCATTTT | ctgatgttttt | CTCATTTTGGAGG | ssRNA | |
| | | | GGAGGGTAATCACAAC | ggctccgtt | GTAATCACAAC | | |
| | | | (SEQ ID. NO: 2454) | (SEQ ID. | (SEQ ID. | | |
| | | | | NO: | NO: 2456) | | |
| | | | | 2455) | | | |
| 1b | ebola_24 | CcaCas13b | gactctgaccactgatgtttttggc | gactctgacc | GTTGGAACTGCT | Ebola | 1b |
| | | | tccgtGTTGGAACTGCTCTCATTTT | actgatgtttt | CTCATTTTGGAGG | ssRNA | |
| | | | GGAGGGTAATCACAAC | tggctccgt | GTAATCACAAC | | |
| | | | (SEQ ID. NO: 2457) | (SEQ ID. | (SEQ ID. | | |
| | | | | NO: | NO: 2459) | | |
| | | | | 2458) | | | |
| 1b | ebola_25 | CcaCas13b | cggactctgaccactgatgtttttg | cggactctg | GTTGGAACTGCT | Ebola | 1b |
| | | | gctccGTTGGAACTGCTCTCATTTT | accactgatg | CTCATTTTGGAGG | ssRNA | |
| | | | GGAGGGTAATCACAAC | tttttggctcc | GTAATCACAAC | | |
| | | | (SEQ ID. NO: 2460) | (SEQ ID. | (SEQ ID. | | |
| | | | | NO: | NO: 2462) | | |
| | | | | 2461) | | | |
| 1b | ebola_26 | CcaCas13b | gccggactctgaccactgatgtttt | gccggactc | GTTGGAACTGCT | Ebola | 1b |
| | | | tggctGTTGGAACTGCTCTCATTTT | tgaccactga | CTCATTTTGGAGG | ssRNA | |
| | | | GGAGGGTAATCACAAC | tgtttttggct | GTAATCACAAC | | |
| | | | (SEQ ID. NO: 2463) | (SEQ ID. | (SEQ ID. | | |
| | | | | NO: | NO: 2465) | | |
| | | | | 2464) | | | |
| 1b | ebola_27 | CcaCas13b | cgccggactctgaccactgatgttt | cgccggact | GTTGGAACTGCT | Ebola | 1b |
| | | | ttggcGTTGGAACTGCTCTCATTTT | ctgaccactg | CTCATTTTGGAGG | ssRNA | |
| | | | GGAGGGTAATCACAAC | atgtttttggc | GTAATCACAAC | | |
| | | | (SEQ ID. NO: 2466) | (SEQ ID. | (SEQ ID. | | |
| | | | | NO: | NO: 2468) | | |
| | | | | 2467) | | | |
| 1b | ebola_28 | CcaCas13b | gcgccggactctgaccactgatgtt | gcgccggac | GTTGGAACTGCT | Ebola | 1b |
| | | | tttggGTTGGAACTGCTCTCATTTT | tctgaccact | CTCATTTTGGAGG | ssRNA | |
| | | | GGAGGGTAATCACAAC | gatgtttttgg | GTAATCACAAC | | |
| | | | (SEQ ID. NO: 2469) | (SEQ ID. | (SEQ ID. | | |
| | | | | NO: | NO: 2471) | | |
| | | | | 2470) | | | |
| 1b | ebola_29 | CcaCas13b | cgcgccggactctgaccactgatgt | cgcgccgga | GTTGGAACTGCT | Ebola | 1b |
| | | | ttttgGTTGGAACTGCTCTCATTTT | ctctgaccac | CTCATTTTGGAGG | ssRNA | |
| | | | GGAGGGTAATCACAAC | tgatgtttttg | GTAATCACAAC | | |
| | | | (SEQ ID. NO: 2472) | (SEQ ID. | (SEQ ID. | | |
| | | | | NO: | NO: 2474) | | |
| | | | | 2473) | | | |
| 1b | ebola_30 | CcaCas13b | ttcgcgccggactctgaccactgat | ttcgcgccg | GTTGGAACTGCT | Ebola | 1b |
| | | | gttttGTTGGAACTGCTCTCATTTT | gactctgacc | CTCATTTTGGAGG | ssRNA | |
| | | | GGAGGGTAATCACAAC | actgatgtttt | GTAATCACAAC | | |
| | | | (SEQ ID. NO: 2475) | (SEQ ID. | (SEQ ID. | | |
| | | | | NO: | NO: 2477) | | |
| | | | | 2476) | | | |
| 1b | ebola_31 | CcaCas13b | agttcgcgccggactctgaccactg | agttcgcgc | GTTGGAACTGCT | Ebola | 1b |
| | | | atgttGTTGGAACTGCTCTCATTTT | cggactctg | CTCATTTTGGAGG | ssRNA | |
| | | | GGAGGGTAATCACAAC | accactgatg | GTAATCACAAC | | |
| | | | (SEQ ID. NO: 2478) | tt (SEQ | (SEQ ID. | | |
| | | | | ID. NO: | NO: 2480) | | |
| | | | | 2479) | | | |
| 1b | ebola_32 | CcaCas13b | aagttcgcgccggactctgaccact | aagttcgcg | GTTGGAACTGCT | Ebola | 1b |
| | | | gatgtGTTGGAACTGCTCTCATTTT | ccggactct | CTCATTTTGGAGG | ssRNA | |
| | | | GGAGGGTAATCACAAC | gaccactgat | GTAATCACAAC | | |
| | | | (SEQ ID. NO: 2481) | gt (SEQ | (SEQ ID. | | |
| | | | | ID. NO: | NO: 2483) | | |
| | | | | 2482) | | | |
| 1b | ebola_33 | CcaCas13b | gaagttcgcgccggactctgaccac | gaagttcgc | GTTGGAACTGCT | Ebola | 1b |
| | | | tgatgGTTGGAACTGCTCTCATTTT | gccggactc | CTCATTTTGGAGG | ssRNA | |
| | | | GGAGGGTAATCACAAC | tgaccactga | GTAATCACAAC | | |
| | | | (SEQ ID. NO: 2484) | tg (SEQ | (SEQ ID. | | |
| | | | | ID. NO: | NO: 2486) | | |
| | | | | 2485) | | | |
| 1b | ebola_34 | CcaCas13b | agaagttcgcgccggactctgacca | agaagttcg | GTTGGAACTGCT | Ebola | 1b |
| | | | ctgatGTTGGAACTGCTCTCATTTT | cgccggact | CTCATTTTGGAGG | ssRNA | |
| | | | GGAGGGTAATCACAAC | ctgaccactg | GTAATCACAAC | | |
| | | | (SEQ ID. NO: 2487) | at (SEQ | (SEQ ID. | | |
| | | | | ID. NO: | NO: 2489) | | |
| | | | | 2488) | | | |
| 1b | ebola_35 | CcaCas13b | gaagaagttcgcgccggactctgac | gaagaagtt | GTTGGAACTGCT | Ebola | 1b |
| | | | cactgGTTGGAACTGCTCTCATTTT | cgcgccgga | CTCATTTTGGAGG | ssRNA | |
| | | | GGAGGGTAATCACAAC | ctctgaccac | GTAATCACAAC | | |
| | | | (SEQ ID. NO: 2490) | tg (SEQ | (SEQ ID. | | |
| | | | | ID. NO: | NO: 2492) | | |
| | | | | 2491) | | | |
| 1b | ebola_36 | CcaCas13b | ggaagaagttcgcgccggactctga | ggaagaagt | GTTGGAACTGCT | Ebola | 1b |
| | | | ccactGTTGGAACTGCTCTCATTTT | tcgcgccgg | CTCATTTTGGAGG | ssRNA | |
| | | | GGAGGGTAATCACAAC | actctgacca | GTAATCACAAC | | |
| | | | (SEQ ID. NO: 2493) | ct (SEQ | (SEQ ID. | | |
| | | | | ID. NO: | NO: 2495) | | |
| | | | | 2494) | | | |
| 1b | ebola_37 | CcaCas13b | tcggaagaagttcgcgccggactct | tcggaagaa | GTTGGAACTGCT | Ebola | 1b |
| | | | gaccaGTTGGAACTGCTCTCATTTT | gttcgcgcc | CTCATTTTGGAGG | ssRNA | |
| | | | GGAGGGTAATCACAAC | ggactctga | GTAATCACAAC | | |
| | | | (SEQ ID. NO: 2496) | cca (SEQ | (SEQ ID. | | |
| | | | | ID. NO: | NO: 2498) | | |
| | | | | 2497) | | | |
| 1b | ebola_38 | CcaCas13b | gtcggaagaagttcgcgccggactc | gtcggaaga | GTTGGAACTGCT | Ebola | 1b |
| | | | tgaccGTTGGAACTGCTCTCATTTT | agttcgcgc | CTCATTTTGGAGG | ssRNA | |
| | | | GGAGGGTAATCACAAC | cggactctg | GTAATCACAAC | | |
| | | | (SEQ ID. NO: 2499) | acc (SEQ | (SEQ ID. | | |
| | | | | ID. NO: | NO: 2501) | | |
| | | | | 2500) | | | |
| 1b | ebola_39 | CcaCas13b | ggtcggaagaagttcgcgccggact | ggtcggaag | GTTGGAACTGCT | Ebola | 1b |
| | | | ctgacGTTGGAACTGCTCTCATTTT | aagttcgcg | CTCATTTTGGAGG | ssRNA | |
| | | | GGAGGGTAATCACAAC | ccggactct | GTAATCACAAC | | |
| | | | (SEQ ID. NO: 2502) | gac (SEQ | (SEQ ID. | | |
| | | | | ID. NO: | NO: 2504) | | |
| | | | | 2503) | | | |
| 1b | ebola_40 | CcaCas13b | gggtcggaagaagttcgcgccggac | gggtcggaa | GTTGGAACTGCT | Ebola | 1b |
| | | | tctgaGTTGGAACTGCTCTCATTTT | gaagttcgc | CTCATTTTGGAGG | ssRNA | |
| | | | GGAGGGTAATCACAAC | gccggactc | GTAATCACAAC | | |
| | | | (SEQ ID. NO: 2505) | tga (SEQ | (SEQ ID. | | |
| | | | | ID. NO: | NO: 2507) | | |
| | | | | 2506) | | | |
| 1b | ebola_41 | CcaCas13b | tgggtcggaagaagttcgcgccgga | tgggtcgga | GTTGGAACTGCT | Ebola | 1b |
| | | | ctctgGTTGGAACTGCTCTCATTTT | agaagttcg | CTCATTTTGGAGG | ssRNA | |
| | | | GGAGGGTAATCACAAC | cgccggact | GTAATCACAAC | | |
| | | | (SEQ ID. NO: 2508) | ctg (SEQ | (SEQ ID. | | |
| | | | | ID. NO: | NO: 2510) | | |
| | | | | 2509) | | | |
| 1b | ebola_42 | CcaCas13b | ccctgggtcggaagaagttcgcgcc | ccctgggtc | GTTGGAACTGCT | Ebola | 1b |
| | | | ggactGTTGGAACTGCTCTCATTTT | ggaagaagt | CTCATTTTGGAGG | ssRNA | |
| | | | GGAGGGTAATCACAAC | tcgcgccgg | GTAATCACAAC | | |
| | | | (SEQ ID. NO: 2511) | act (SEQ | (SEQ ID. | | |
| | | | | ID. NO: | NO: 2513) | | |
| | | | | 2512) | | | |
| 1b | ebola_43 | CcaCas13b | tccctgggtcggaagaagttcgcgc | tccctgggtc | GTTGGAACTGCT | Ebola | 1b |
| | | | cggacGTTGGAACTGCTCTCATTTT | ggaagaagt | CTCATTTTGGAGG | ssRNA | |
| | | | GGAGGGTAATCACAAC | tcgcgccgg | GTAATCACAAC | | |
| | | | (SEQ ID. NO: 2514) | ac (SEQ | (SEQ ID. | | |
| | | | | ID. NO: | NO: 2516) | | |
| | | | | 2515) | | | |
| 1b | ebola_44 | CcaCas13b | gtccctgggtcggaagaagttcgcg | gtccctgggt | GTTGGAACTGCT | Ebola | 1b |
| | | | ccggaGTTGGAACTGCTCTCATTTT | cggaagaag | CTCATTTTGGAGG | ssRNA | |
| | | | GGAGGGTAATCACAAC | ttcgcgccg | GTAATCACAAC | | |
| | | | (SEQ ID. NO: 2517) | ga (SEQ | (SEQ ID. | | |
| | | | | ID. NO: | NO: 2519) | | |
| | | | | 2518) | | | |
| 1b | ebola_45 | CcaCas13b | ggtccctgggtcggaagaagttcgc | ggtccctgg | GTTGGAACTGCT | Ebola | 1b |
| | | | gccggGTTGGAACTGCTCTCATTTT | gtcggaaga | CTCATTTTGGAGG | ssRNA | |
| | | | GGAGGGTAATCACAAC | agttcgcgc | GTAATCACAAC | | |
| | | | (SEQ ID. NO: 2520) | cgg (SEQ | (SEQ ID. | | |
| | | | | ID. NO: | NO: 2522) | | |
| | | | | 2521) | | | |
| 1b | ebola_46 | CcaCas13b | tggtccctgggtcggaagaagttcg | tggtccctgg | GTTGGAACTGCT | Ebola | 1b |
| | | | cgccgGTTGGAACTGCTCTCATTTT | gtcggaaga | CTCATTTTGGAGG | ssRNA | |
| | | | GGAGGGTAATCACAAC | agttcgcgc | GTAATCACAAC | | |
| | | | (SEQ ID. NO: 2523) | cg (SEQ | (SEQ ID. | | |
| | | | | ID. NO: | NO: 2525) | | |
| | | | | 2524) | | | |
| 1b | ebola_47 | CcaCas13b | ttggtccctgggtcggaagaagttc | ttggtccctg | GTTGGAACTGCT | Ebola | 1b |
| | | | gcgccGTTGGAACTGCTCTCATTTT | ggtcggaag | CTCATTTTGGAGG | ssRNA | |
| | | | GGAGGGTAATCACAAC | aagttcgcg | GTAATCACAAC | | |
| | | | (SEQ ID. NO: 2526) | cc (SEQ | (SEQ ID. | | |
| | | | | ID. NO: | NO: 2528) | | |
| | | | | 2527) | | | |
| 1b | ebola_48 | CcaCas13b | gtgttggtccctgggtcggaagaag | gtgttggtcc | GTTGGAACTGCT | Ebola | 1b |
| | | | ttcgcGTTGGAACTGCTCTCATTTT | ctgggtcgg | CTCATTTTGGAGG | ssRNA | |
| | | | GGAGGGTAATCACAAC | aagaagttc | GTAATCACAAC | | |
| | | | (SEQ ID. NO: 2529) | gc (SEQ | (SEQ ID. | | |
| | | | | ID. NO: | NO: 2531) | | |
| | | | | 2530) | | | |
| 1b | ebola_49 | CcaCas13b | tgtgttggtccctgggtcggaagaa | tgtgttggtc | GTTGGAACTGCT | Ebola | 1b |
| | | | gttcgGTTGGAACTGCTCTCATTTT | cctgggtcg | CTCATTTTGGAGG | ssRNA | |
| | | | GGAGGGTAATCACAAC | gaagaagtt | GTAATCACAAC | | |
| | | | (SEQ ID. NO: 2532) | cg (SEQ | (SEQ ID. | | |
| | | | | ID. NO: | NO: 2534) | | |
| | | | | 2533) | | | |
| 1b | ebola_50 | CcaCas13b | ttgtgttggtccctgggtcggaaga | ttgtgttggtc | GTTGGAACTGCT | Ebola | 1b |
| | | | agttcGTTGGAACTGCTCTCATTTT | cctgggtcg | CTCATTTTGGAGG | ssRNA | |
| | | | GGAGGGTAATCACAAC | gaagaagtt | GTAATCACAAC | | |
| | | | (SEQ ID. NO: 2535) | c (SEQ | (SEQ ID. | | |
| | | | | ID. NO: | NO: 2537) | | |
| | | | | 2536) | | | |
| 1b | ebola_51 | CcaCas13b | tgttgtgttggtccctgggtcggaa | tgttgtgttgg | GTTGGAACTGCT | Ebola | 1b |
| | | | gaagtGTTGGAACTGCTCTCATTTT | tccctgggtc | CTCATTTTGGAGG | ssRNA | |
| | | | GGAGGGTAATCACAAC | ggaagaagt | GTAATCACAAC | | |
| | | | (SEQ ID. NO: 2538) | (SEQ ID. | (SEQ ID. | | |
| | | | | NO: | NO: 2540) | | |
| | | | | 2539) | | | |
| 1b | ebola_52 | CcaCas13b | ttgttgtgttggtccctgggtcgga | ttgttgtgttg | GTTGGAACTGCT | Ebola | 1b |
| | | | agaagGTTGGAACTGCTCTCATTTT | gtccctgggt | CTCATTTTGGAGG | ssRNA | |
| | | | GGAGGGTAATCACAAC | cggaagaag | GTAATCACAAC | | |
| | | | (SEQ ID. NO: 2541) | (SEQ ID. | (SEQ ID. | | |
| | | | | NO: | NO: 2543) | | |
| | | | | 2542) | | | |
| 1b | ebola_53 | CcaCas13b | gttgttgtgttggtccctgggtcgg | gttgttgtgtt | GTTGGAACTGCT | Ebola | 1b |
| | | | aagaaGTTGGAACTGCTCTCATTTT | ggtccctgg | CTCATTTTGGAGG | ssRNA | |
| | | | GGAGGGTAATCACAAC | gtcggaaga | GTAATCACAAC | | |
| | | | (SEQ ID. NO: 2544) | a (SEQ | (SEQ ID. | | |
| | | | | ID. NO: | NO: 2546) | | |
| | | | | 2545) | | | |
| 1b | ebola_54 | CcaCas13b | tcagttgttgtgttggtccctgggt | tcagttgttgt | GTTGGAACTGCT | Ebola | 1b |
| | | | cggaaGTTGGAACTGCTCTCATTTT | gttggtccct | CTCATTTTGGAGG | ssRNA | |
| | | | GGAGGGTAATCACAAC | gggtcggaa | GTAATCACAAC | | |
| | | | (SEQ ID. NO: 2547) | (SEQ ID. | (SEQ ID. | | |
| | | | | NO: | NO: 2549) | | |
| | | | | 2548) | | | |
| 1b | ebola_55 | CcaCas13b | ttcagttgttgtgttggtccctggg | ttcagttgttg | GTTGGAACTGCT | Ebola | 1b |
| | | | tcggaGTTGGAACTGCTCTCATTTT | tgttggtccct | CTCATTTTGGAGG | ssRNA | |
| | | | GGAGGGTAATCACAAC | gggtcgga | GTAATCACAAC | | |
| | | | (SEQ ID. NO: 2550) | (SEQ ID. | (SEQ ID. | | |
| | | | | NO: | NO: 2552) | | |
| | | | | 2551) | | | |
| 1b | ebola_56 | CcaCas13b | cttcagttgttgtgttggtccctgg | cttcagttgtt | GTTGGAACTGCT | Ebola | 1b |
| | | | gtcggGTTGGAACTGCTCTCATTTT | gtgttggtcc | CTCATTTTGGAGG | ssRNA | |
| | | | GGAGGGTAATCACAAC | ctgggtcgg | GTAATCACAAC | | |
| | | | (SEQ ID. NO: 2553) | (SEQ ID. | (SEQ ID. | | |
| | | | | NO: | NO: 2555) | | |
| | | | | 2554) | | | |
| 1b | ebola_57 | CcaCas13b | tcttcagttgttgtgttggtccctg | tcttcagttgt | GTTGGAACTGCT | Ebola | 1b |
| | | | ggtcgGTTGGAACTGCTCTCATTTT | tgtgttggtc | CTCATTTTGGAGG | ssRNA | |
| | | | GGAGGGTAATCACAAC | cctgggtcg | GTAATCACAAC | | |
| | | | (SEQ ID. NO: 2556) | (SEQ ID. | (SEQ ID. | | |
| | | | | NO: | NO: 2558) | | |
| | | | | 2557) | | | |
| 1b | ebola_58 | CcaCas13b | gtcttcagttgttgtgttggtccct | gtcttcagttg | GTTGGAACTGCT | Ebola | 1b |
| | | | gggtcGTTGGAACTGCTCTCATTTT | ttgtgttggtc | CTCATTTTGGAGG | ssRNA | |
| | | | GGAGGGTAATCACAAC | cctgggtc | GTAATCACAAC | | |
| | | | (SEQ ID. NO: 2559) | (SEQ ID. | (SEQ ID. | | |
| | | | | NO: | NO: 2561) | | |
| | | | | 2560) | | | |
| 1b | ebola_59 | CcaCas13b | ggtcttcagttgttgtgttggtccc | ggtcttcagtt | GTTGGAACTGCT | Ebola | 1b |
| | | | tgggtGTTGGAACTGCTCTCATTTT | gttgtgttggt | CTCATTTTGGAGG | ssRNA | |
| | | | GGAGGGTAATCACAAC | ccctgggt | GTAATCACAAC | | |
| | | | (SEQ ID. NO: 2562) | (SEQ ID. | (SEQ ID. | | |
| | | | | NO: | NO: 2564) | | |
| | | | | 2563) | | | |
| 1b | ebola_60 | CcaCas13b | tgtggtcttcagttgttgtgttggt | tgtggtcttca | GTTGGAACTGCT | Ebola | 1b |
| | | | ccctgGTTGGAACTGCTCTCATTTT | gttgttgtgtt | CTCATTTTGGAGG | ssRNA | |
| | | | GGAGGGTAATCACAAC | ggtccctg | GTAATCACAAC | | |
| | | | (SEQ ID. NO: 2565) | (SEQ ID. | (SEQ ID. | | |
| | | | | NO: | NO: 2567) | | |
| | | | | 2566) | | | |
| 1b | ebola_61 | CcaCas13b | ttgtggtcttcagttgttgtgttgg | ttgtggtcttc | GTTGGAACTGCT | Ebola | 1b |
| | | | tccctGTTGGAACTGCTCTCATTTT | agttgttgtgt | CTCATTTTGGAGG | ssRNA | |
| | | | GGAGGGTAATCACAAC | tggtccct | GTAATCACAAC | | |
| | | | (SEQ ID. NO: 2568) | (SEQ ID. | (SEQ ID. | | |
| | | | | NO: | NO: 2570) | | |
| | | | | 2569) | | | |
| 1b | ebola_62 | CcaCas13b | tttgtggtcttcagttgttgtgttg | ttgtggtctt | GTTGGAACTGCT | Ebola | 1b |
| | | | gtcccGTTGGAACTGCTCTCATTTT | cagttgttgt | CTCATTTTGGAGG | ssRNA | |
| | | | GGAGGGTAATCACAAC | gttggtccc | GTAATCACAAC | | |
| | | | (SEQ ID. NO: 2571) | (SEQ ID. | (SEQ ID. | | |
| | | | | NO: | NO: 2573) | | |
| | | | | 2572) | | | |
| 1b | ebola_63 | CcaCas13b | ttttgtggtcttcagttgttgtgtt | tttgtggtctt | GTTGGAACTGCT | Ebola | 1b |
| | | | ggtccGTTGGAACTGCTCTCATTTT | cagttgttgt | CTCATTTTGGAGG | ssRNA | |
| | | | GGAGGGTAATCACAAC | gttggtcc | GTAATCACAAC | | |
| | | | (SEQ ID. NO: 2574) | (SEQ ID. | (SEQ ID. | | |
| | | | | NO: | NO: 2576) | | |
| | | | | 2575) | | | |
| 1b | ebola_64 | CcaCas13b | gattttgtggtcttcagttgttgtg | gattttgtggt | GTTGGAACTGCT | Ebola | 1b |
| | | | ttggtGTTGGAACTGCTCTCATTTT | cttcagttgtt | CTCATTTTGGAGG | ssRNA | |
| | | | GGAGGGTAATCACAAC | gtgttggt | GTAATCACAAC | | |
| | | | (SEQ ID. NO: 2577) | (SEQ ID. | (SEQ ID. | | |
| | | | | NO: | NO: 2579) | | |
| | | | | 2578) | | | |
| 1b | ebola_65 | CcaCas13b | tgattttgtggtcttcagttgttgt | tgattttgtgg | GTTGGAACTGCT | Ebola | 1b |
| | | | gttggGTTGGAACTGCTCTCATTTT | tcttcagttgt | CTCATTTTGGAGG | ssRNA | |
| | | | GGAGGGTAATCACAAC | tgtgttgg | GTAATCACAAC | | |
| | | | (SEQ ID. NO: 2580) | (SEQ ID. | (SEQ ID. | | |
| | | | | NO: | NO: 2582) | | |
| | | | | 2581) | | | |
| 1b | ebola_66 | CcaCas13b | atgattttgtggtcttcagttgttg | atgattttgtg | GTTGGAACTGCT | Ebola | 1b |
| | | | tgttgGTTGGAACTGCTCTCATTTT | gtcttcagttg | CTCATTTTGGAGG | ssRNA | |
| | | | GGAGGGTAATCACAAC | ttgtgttg | GTAATCACAAC | | |
| | | | (SEQ ID. NO: 2583) | (SEQ ID. | (SEQ ID. | | |
| | | | | NO: | NO: 2585) | | |
| | | | | 2584) | | | |
| 1b | ebola_67 | CcaCas13b | ccatgattttgtggtcttcagttgt | ccatgattttg | GTTGGAACTGCT | Ebola | 1b |
| | | | tgtgtGTTGGAACTGCTCTCATTTT | tggtcttcagt | CTCATTTTGGAGG | ssRNA | |
| | | | GGAGGGTAATCACAAC | tgttgtgt | GTAATCACAAC | | |
| | | | (SEQ ID. NO: 2586) | (SEQ ID. | (SEQ ID. | | |
| | | | | NO: | NO: 2588) | | |
| | | | | 2587) | | | |
| 1b | ebola_68 | CcaCas13b | agccatgattttgtggtcttcagtt | agccatgatt | GTTGGAACTGCT | Ebola | 1b |
| | | | gttgtGTTGGAACTGCTCTCATTTT | ttgtggtcttc | CTCATTTTGGAGG | ssRNA | |
| | | | GGAGGGTAATCACAAC | agttgttgt | GTAATCACAAC | | |
| | | | (SEQ ID. NO: 2589) | (SEQ ID. | (SEQ ID. | | |
| | | | | NO: | NO: 2591) | | |
| | | | | 2590) | | | |
| 1b | ebola_69 | CcaCas13b | aagccatgattttgtggtcttcagt | aagccatgat | GTTGGAACTGCT | Ebola | 1b |
| | | | tgttgGTTGGAACTGCTCTCATTTT | tttgtggtctt | CTCATTTTGGAGG | ssRNA | |
| | | | GGAGGGTAATCACAAC | cagttgttg | GTAATCACAAC | | |
| | | | (SEQ ID. NO: 2592) | (SEQ ID. | (SEQ ID. | | |
| | | | | NO: | NO: 2594) | | |
| | | | | 2593) | | | |
| 1b | ebola_70 | CcaCas13b | gaagccatgattttgtggtcttcag | gaagccatg | GTTGGAACTGCT | Ebola | 1b |
| | | | ttgttGTTGGAACTGCTCTCATTTT | attttgtggtc | CTCATTTTGGAGG | ssRNA | |
| | | | GAGGGTAATCACAAC | ttcagttgtt | GTAATCACAAC | | |
| | | | G(SEQ ID. NO: 2595) | (SEQ ID. | (SEQ ID. | | |
| | | | | NO: | NO: 2597) | | |
| | | | | 2596) | | | |
| 1b | ebola_71 | CcaCas13b | tgaagccatgattttgtggtcttca | tgaagccat | GTTGGAACTGCT | Ebola | 1b |
| | | | gttgtGTTGGAACTGCTCTCATTTT | gattttgtggt | CTCATTTTGGAGG | ssRNA | |
| | | | GGAGGGTAATCACAAC | cttcagttgt | GTAATCACAAC | | |
| | | | (SEQ ID. NO: 2598) | (SEQ ID. | (SEQ ID. | | |
| | | | | NO: | NO: 2600) | | |
| | | | | 2599) | | | |
| 1b | ebola_72 | CcaCas13b | ttctgaagccatgattttgtggtct | ttctgaagcc | GTTGGAACTGCT | Ebola | 1b |
| | | | tcagtGTTGGAACTGCTCTCATTTT | atgattttgtg | CTCATTTTGGAGG | ssRNA | |
| | | | GGAGGGTAATCACAAC | gtcttcagt | GTAATCACAAC | | |
| | | | (SEQ ID. NO: 2601) | (SEQ ID. | (SEQ ID. | | |
| | | | | NO: | NO: 2603) | | |
| | | | | 2602) | | | |
| 1b | ebola_73 | CcaCas13b | tttctgaagccatgattttgtggtc | tttctgaagc | GTTGGAACTGCT | Ebola | 1b |
| | | | ttcagGTTGGAACTGCTCTCATTTT | catgattttgt | CTCATTTTGGAGG | ssRNA | |
| | | | GGAGGGTAATCACAAC | ggtcttcag | GTAATCACAAC | | |
| | | | (SEQ ID. NO: 2604) | (SEQ ID. | (SEQ ID. | | |
| | | | | NO: | NO: 2606) | | |
| | | | | 2605) | | | |
| 1b | ebola_74 | CcaCas13b | attttctgaagccatgattttgtgg | attttctgaag | GTTGGAACTGCT | Ebola | 1b |
| | | | tcttcGTTGGAACTGCTCTCATTTT | ccatgattttg | CTCATTTTGGAGG | ssRNA | |
| | | | GGAGGGTAATCACAAC | tggtcttc | GTAATCACAAC | | |
| | | | (SEQ ID. NO: 2607) | (SEQ ID. | (SEQ ID. | | |
| | | | | NO: | NO: 2609) | | |
| | | | | 2608) | | | |
| 1b | ebola_75 | CcaCas13b | aattttctgaagccatgattttgtg | aattttctgaa | GTTGGAACTGCT | Ebola | 1b |
| | | | gtcttGTTGGAACTGCTCTCATTTT | gccatgatttt | CTCATTTTGGAGG | ssRNA | |
| | | | GGAGGGTAATCACAAC | gtggtctt | GTAATCACAAC | | |
| | | | (SEQ ID. NO: 2610) | (SEQ ID. | (SEQ ID. | | |
| | | | | NO: | NO: 2612) | | |
| | | | | 2611) | | | |
| 1b | ebola_76 | CcaCas13b | gaattttctgaagccatgattttgt | gaattttctga | GTTGGAACTGCT | Ebola | 1b |
| | | | ggtctGTTGGAACTGCTCTCATTTT | agccatgatt | CTCATTTTGGAGG | ssRNA | |
| | | | GGAGGGTAATCACAAC | ttgtggtct | GTAATCACAAC | | |
| | | | (SEQ ID. NO: 2613) | (SEQ ID. | (SEQ ID. | | |
| | | | | NO: | NO: 2615) | | |
| | | | | 2614) | | | |
| 1b | ebola_77 | CcaCas13b | aggaattttctgaagccatgatttt | aggaattttct | GTTGGAACTGCT | Ebola | 1b |
| | | | gtggtGTTGGAACTGCTCTCATTTT | gaagccatg | CTCATTTTGGAGG | ssRNA | |
| | | | GGAGGGTAATCACAAC | attttgtggt | GTAATCACAAC | | |
| | | | (SEQ ID. NO: 2616) | (SEQ ID. | (SEQ ID. | | |
| | | | | NO: | NO: 2618) | | |
| | | | | 2617) | | | |
| 1b | ebola_78 | CcaCas13b | agaggaattttctgaagccatgatt | agaggaattt | GTTGGAACTGCT | Ebola | 1b |
| | | | ttgtgGTTGGAACTGCTCTCATTTT | tctgaagcca | CTCATTTTGGAGG | ssRNA | |
| | | | GGAGGGTAATCACAAC | tgattttgtg | GTAATCACAAC | | |
| | | | (SEQ ID. NO: 2619) | (SEQ ID. | (SEQ ID. | | |
| | | | | NO: | NO: 2621) | | |
| | | | | 2620) | | | |
| 1b | ebola_79 | CcaCas13b | cagaggaattttctgaagccatgat | cagaggaat | GTTGGAACTGCT | Ebola | 1b |
| | | | tttgtGTTGGAACTGCTCTCATTTT | tttctgaagc | CTCATTTTGGAGG | ssRNA | |
| | | | GGAGGGTAATCACAAC | catgattttgt | GTAATCACAAC | | |
| | | | (SEQ ID. NO: 2622) | (SEQ ID. | (SEQ ID. | | |
| | | | | NO: | NO: 2624) | | |
| | | | | 2623) | | | |
| 1b | ebola_80 | CcaCas13b | gcagaggaattttctgaagccatga | gcagaggaa | GTTGGAACTGCT | Ebola | 1b |
| | | | ttttgGTTGGAACTGCTCTCATTTT | ttttctgaagc | CTCATTTTGGAGG | ssRNA | |
| | | | GGAGGGTAATCACAAC | catgattttg | GTAATCACAAC | | |
| | | | (SEQ ID. NO: 2625) | (SEQ ID. | (SEQ ID. | | |
| | | | | NO: | NO: 2627) | | |
| | | | | 2626) | | | |
| 1b | ebola_81 | CcaCas13b | tgcagaggaattttctgaagccatg | tgcagagga | GTTGGAACTGCT | Ebola | 1b |
| | | | attttGTTGGAACTGCTCTCATTTT | attttctgaag | CTCATTTTGGAGG | ssRNA | |
| | | | GGAGGGTAATCACAAC | ccatgatttt | GTAATCACAAC | | |
| | | | (SEQ ID. NO: 2628) | (SEQ ID. | (SEQ ID. | | |
| | | | | NO: | NO: 2630) | | |
| | | | | 2629) | | | |
| 1b | ebola_82 | CcaCas13b | cattgcagaggaattttctgaagcc | cattgcaga | GTTGGAACTGCT | Ebola | 1b |
| | | | atgatGTTGGAACTGCTCTCATTTT | ggaattttctg | CTCATTTTGGAGG | ssRNA | |
| | | | GGAGGGTAATCACAAC | aagccatgat | GTAATCACAAC | | |
| | | | (SEQ ID. NO: 2631) | (SEQ ID. | (SEQ ID. | | |
| | | | | NO: | NO: 2633) | | |
| | | | | 2632) | | | |
| 1b | ebola_83 | CcaCas13b | ccattgcagaggaattttctgaagc | ccattgcaga | GTTGGAACTGCT | Ebola | 1b |
| | | | catgaGTTGGAACTGCTCTCATTTT | ggaattttctg | CTCATTTTGGAGG | ssRNA | |
| | | | GGAGGGTAATCACAAC | aagccatga | GTAATCACAAC | | |
| | | | (SEQ ID. NO: 2634) | (SEQ ID. | (SEQ ID. | | |
| | | | | NO: | NO: 2636) | | |
| | | | | 2635) | | | |
| 1b | ebola_84 | CcaCas13b | accattgcagaggaattttctgaag | accattgcag | GTTGGAACTGCT | Ebola | 1b |
| | | | ccatgGTTGGAACTGCTCTCATTTT | aggaattttct | CTCATTTTGGAGG | ssRNA | |
| | | | GGAGGGTAATCACAAC | gaagccatg | GTAATCACAAC | | |
| | | | (SEQ ID. NO: 2637) | (SEQ ID. | (SEQ ID. | | |
| | | | | NO: | NO: 2639) | | |
| | | | | 2638) | | | |
| 1b | ebola_85 | CcaCas13b | aaccattgcagaggaattttctgaa | aaccattgca | GTTGGAACTGCT | Ebola | 1b |
| | | | gccatGTTGGAACTGCTCTCATTTT | gaggaatttt | CTCATTTTGGAGG | ssRNA | |
| | | | GGAGGGTAATCACAAC | ctgaagccat | GTAATCACAAC | | |
| | | | (SEQ ID. NO: 2640) | (SEQ ID. | (SEQ ID. | | |
| | | | | NO: | NO: 2642) | | |
| | | | | 2641) | | | |
| 1b | ebola_86 | CcaCas13b | ttgaaccattgcagaggaattttct | ttgaaccatt | GTTGGAACTGCT | Ebola | 1b |
| | | | gaagcGTTGGAACTGCTCTCATTTT | gcagaggaa | CTCATTTTGGAGG | ssRNA | |
| | | | GGAGGGTAATCACAAC | ttttctgaagc | GTAATCACAAC | | |
| | | | (SEQ ID. NO: 2643) | (SEQ ID. | (SEQ ID. | | |
| | | | | NO: | NO: 2645) | | |
| | | | | 2644) | | | |
| 1b | ebola_87 | CcaCas13b | acttgaaccattgcagaggaatttt | acttgaacca | GTTGGAACTGCT | Ebola | 1b |
| | | | ctgaaGTTGGAACTGCTCTCATTTT | ttgcagagg | CTCATTTTGGAGG | ssRNA | |
| | | | GGAGGGTAATCACAAC | aattttctgaa | GTAATCACAAC | | |
| | | | (SEQ ID. NO: 2646) | (SEQ ID. | (SEQ ID. | | |
| | | | | NO: | NO: 2648) | | |
| | | | | 2647) | | | |
| 1b | ebola_88 | CcaCas13b | cacttgaaccattgcagaggaattt | cacttgaacc | GTTGGAACTGCT | Ebola | 1b |
| | | | tctgaGTTGGAACTGCTCTCATTTT | attgcagag | CTCATTTTGGAGG | ssRNA | |
| | | | GGAGGGTAATCACAAC | gaattttctga | GTAATCACAAC | | |
| | | | (SEQ ID. NO: 2649) | (SEQ ID. | (SEQ ID. | | |
| | | | | NO: | NO: 2651) | | |
| | | | | 2650) | | | |
| 1b | ebola_89 | CcaCas13b | tgcacttgaaccattgcagaggaat | tgcacttgaa | GTTGGAACTGCT | Ebola | 1b |
| | | | tttctGTTGGAACTGCTCTCATTTT | ccattgcaga | CTCATTTTGGAGG | ssRNA | |
| | | | GGAGGGTAATCACAAC | ggaattttct | GTAATCACAAC | | |
| | | | (SEQ ID. NO: 2652) | (SEQ ID. | (SEQ ID. | | |
| | | | | NO: | NO: 2654) | | |
| | | | | 2653) | | | |
| 1b | ebola_90 | CcaCas13b | gtgcacttgaaccattgcagaggaa | gtgcacttga | GTTGGAACTGCT | Ebola | 1b |
| | | | ttttcGTTGGAACTGCTCTCATTTT | accattgcag | CTCATTTTGGAGG | ssRNA | |
| | | | GGAGGGTAATCACAAC | aggaattttc | GTAATCACAAC | | |
| | | | (SEQ ID. NO: 2655) | (SEQ ID. | (SEQ ID. | | |
| | | | | NO: | NO: 2657) | | |
| | | | | 2656) | | | |
| 1b | ebola_91 | CcaCas13b | ctgtgcacttgaaccattgcagagg | ctgtgcactt | GTTGGAACTGCT | Ebola | 1b |
| | | | aatttGTTGGAACTGCTCTCATTTT | gaaccattgc | CTCATTTTGGAGG | ssRNA | |
| | | | GGAGGGTAATCACAAC | agaggaattt | GTAATCACAAC | | |
| | | | (SEQ ID. NO: 2658) | (SEQ ID. | (SEQ ID. | | |
| | | | | NO: | NO: 2660) | | |
| | | | | 2659) | | | |
| 1b | ebola_92 | CcaCas13b | actgtgcacttgaaccattgcagag | actgtgcact | GTTGGAACTGCT | Ebola | 1b |
| | | | gaattGTTGGAACTGCTCTCATTTT | tgaaccattg | CTCATTTTGGAGG | ssRNA | |
| | | | GGAGGGTAATCACAAC | cagaggaat | GTAATCACAAC | | |
| | | | (SEQ ID. NO: 2661) | t (SEQ | (SEQ ID. | | |
| | | | | ID. NO: | NO: 2663) | | |
| | | | | 2662) | | | |
| 1b | ebola_93 | CcaCas13b | tgactgtgcacttgaaccattgcag | tgactgtgca | GTTGGAACTGCT | Ebola | 1b |
| | | | aggaaGTTGGAACTGCTCTCATTTT | cttgaaccat | CTCATTTTGGAGG | ssRNA | |
| | | | GGAGGGTAATCACAAC | tgcagagga | GTAATCACAAC | | |
| | | | (SEQ ID. NO: 2664) | a (SEQ | (SEQ ID. | | |
| | | | | ID. NO: | NO: 2666) | | |
| | | | | 2665) | | | |
| 1b | ebola_94 | CcaCas13b | ttgactgtgcacttgaaccattgca | ttgactgtgc | GTTGGAACTGCT | Ebola | 1b |
| | | | gaggaGTTGGAACTGCTCTCATTTT | acttgaacca | CTCATTTTGGAGG | ssRNA | |
| | | | GGAGGGTAATCACAAC | ttgcagagg | GTAATCACAAC | | |
| | | | (SEQ ID. NO: 2667) | a (SEQ | (SEQ ID. | | |
| | | | | ID. NO: | NO: 2669) | | |
| | | | | 2668) | | | |
| 2a | thermo- | LwaCas13a | GATTTAGACTACCCCAAAA | TATCAA | GATTTAGACTAC | ther- | 2a |
| | nuclease | | ACGAAGGGGACTAAAACT | CCAATA | CCCAAAAACGAA | mo- | |
| | valida- | | ATCAACCAATAATAGTCTG | ATAGTC | GGGGACTAAAAC | nu- | |
| | tion | | AATGTCAT | TGAATG | (SEQ ID. | clease | |
| | LwaCas13 | | (SEQ ID. NO: 2670) | TCAT | NO: 2672) | | |
| | a 1 | | | (SEQ ID. | | | |
| | | | | NO: | | | |
| | | | | 2671) | | | |
| 2a | thermo- | LwaCas13a | GATTTAGACTACCCCAAAA | ATGTCA | GATTTAGACTAC | ther- | 2a |
| | nuclease | | ACGAAGGGGACTAAAACA | TTGGTT | CCCAAAAACGAA | mo- | |
| | valida- | | TGTCATTGGTTGACCTTTGT | GACCTT | GGGGACTAAAAC | nu- | |
| | tion | | ACATTAA | TGTACA | (SEQ ID. | clease | |
| | LwaCas13 | | (SEQ ID. NO: 2673) | TTAA | NO: 2675) | | |
| | a 2 | | | (SEQ ID. | | | |
| | | | | NO: | | | |
| | | | | 2674) | | | |
| 2a | thermo- | LwaCas13a | GATTTAGACTACCCCAAAA | TTAGGA | GATTTAGACTAC | ther- | 2a |
| | nuclease | | ACGAAGGGGACTAAAACTT | TGCTTT | CCCAAAAACGAA | mo- | |
| | valida- | | AGGATGCTTTGTTTCAGGT | GTTTCA | GGGGACTAAAAC | nu- | |
| | tion | | GTATCAA | GGTGTA | (SEQ ID. | clease | |
| | LwaCas13 | | (SEQ ID. NO: 2676) | TCAA | NO: 2678) | | |
| | a 3 | | | (SEQ ID. | | | |
| | | | | NO: | | | |
| | | | | 2677) | | | |
| 2a | thermo- | LwaCas13a | GATTTAGACTACCCCAAAA | TTTCTC | GATTTAGACTAC | ther- | 2a |
| | nuclease | | ACGAAGGGGACTAAAACTT | TACACC | CCCAAAAACGAA | mo- | |
| | valida- | | TCTCTACACCTTTTTTAGGA | AGGATG | GGGGACTAAAAC | nu- | |
| | tion | | TGCTTT | CTTT | (SEQ ID. | clease | |
| | LwaCas13 | | (SEQ ID. NO: 2679) | (SEQ ID. | NO: 2681) | | |
| | a 4 | | | NO: | | | |
| | | | | 2680) | | | |
| 2a | thermo- | LwaCas13a | GATTTAGACTACCCCAAAA | TGTCAT | GATTTAGACTAC | ther- | 2a |
| | nuclease | | ACGAAGGGGACTAAAACT | TGGTTG | CCCAAAAACGAA | mo- | |
| | valida- | | GTCATTGGTTGACCTTTGT | ACCTTT | GGGGACTAAAAC | nu- | |
| | tion | | ACATTAAT | GTACAT | (SEQ ID. | clease | |
| | LwaCas13 | | (SEQ ID. NO: 2682) | TAAT | NO: 2684) | | |
| | a 5 | | | (SEQ ID. | | | |
| | | | | NO: | | | |
| | | | | 2683) | | | |
| 2a | thermo- | LwaCas13a | GATTTAGACTACCCCAAAA | ATAGTC | GATTTAGACTAC | ther- | 2a |
| | nuclease | | ACGAAGGGGACTAAAACA | TGAATG | CCCAAAAACGAA | mo- | |
| | valida- | | TAGTCTGAATGTCATTGGT | TCATTG | GGGGACTAAAAC | nu- | |
| | tion | | TGACCTTT | GTTGAC | (SEQ ID. | clease | |
| | LwaCas13 | | (SEQ ID. NO: 2685) | CTTT | NO: 2687) | | |
| | a 6 | | | (SEQ ID. | | | |
| | | | | NO: | | | |
| | | | | 2686) | | | |
| 2a | thermo- | LwaCas13a | GATTTAGACTACCCCAAAA | AGTCTG | GATTTAGACTAC | ther- | 2a |
| | nuclease | | ACGAAGGGGACTAAAACA | AATGTC | CCCAAAAACGAA | mo- | |
| | valida- | | GTCTGAATGTCATTGGTTG | ATTGGT | GGGGACTAAAAC | nu- | |
| | tion | | ACCTTTGT | TGACCT | (SEQ ID. | clease | |
| | LwaCas13 | | (SEQ ID. NO: 2688) | TTGT | NO: 2690) | | |
| | a 7 | | | (SEQ ID. | | | |
| | | | | NO: | | | |
| | | | | 2689) | | | |
| 2a | thermo- | LwaCas13a | GATTTAGACTACCCCAAAA | TACATT | GATTTAGACTAC | ther- | 2a |
| | nuclease | | ACGAAGGGGACTAAAACT | AATTTA | CCCAAAAACGAA | mo- | |
| | valida- | | ACATTAATTTAACAGTATC | ACAGTA | GGGGACTAAAAC | nu- | |
| | tion | | ACCATCAA | TCACCA | (SEQ ID. | clease | |
| | LwaCas13 | | (SEQ ID. NO: 2691) | TCAA | NO: 2693) | | |
| | a 8 | | | (SEQ ID. | | | |
| | | | | NO: | | | |
| | | | | 2692) | | | |
| 2a | thermo- | LwaCas13a | GATTTAGACTACCCCAAAA | ATGCTT | GATTTAGACTAC | ther- | 2a |
| | nuclease | | ACGAAGGGGACTAAAACA | TGTTTC | CCCAAAAACGAA | mo- | |
| | valida- | | TGCTTTGTTTCAGGTGTATC | AGGTGT | GGGGACTAAAAC | nu- | |
| | tion | | AACCAAT | ATCAAC | (SEQ ID. | clease | |
| | LwaCas13 | | (SEQ ID. NO: 2694) | CAAT | NO: 2696) | | |
| | a 9 | | | (SEQ ID. | | | |
| | | | | NO: | | | |
| | | | | 2695) | | | |
| 2a | thermo- | LwaCas13a | GATTTAGACTACCCCAAAA | AGGATG | GATTTAGACTAC | ther- | 2a |
| | nuclease | | ACGAAGGGGACTAAAACA | CTTTGT | CCCAAAAACGAA | mo- | |
| | valida- | | GGATGCTTTGTTTCAGGTG | TTCAGG | GGGGACTAAAAC | nu- | |
| | tion | | TATCAACC | TGTATC | (SEQ ID. | clease | |
| | LwaCas13 | | (SEQ ID. NO: 2697) | AACC | NO: 2699) | | |
| | a 10 | | | (SEQ ID. | | | |
| | | | | NO: | | | |
| | | | | 2698) | | | |
| 2a | thermo- | LwaCas13a | GATTTAGACTACCCCAAAA | CATATT | GATTTAGACTAC | ther- | 2a |
| | nuclease | | ACGAAGGGGACTAAAACC | TCTCTA | CCCAAAAACGAA | mo- | |
| | valida- | | ATATTTCTCTACACCTTTTT | CACCTT | GGGGACTAAAAC | nu- | |
| | tion | | TAGGATG | TTTTAG | (SEQ ID. | clease | |
| | LwaCas13 | | (SEQ ID. NO: 2700) | GATG | NO: 2702) | | |
| | a 11 | | | (SEQ ID. | | | |
| | | | | NO: | | | |
| | | | | 2701) | | | |
| 2a | thermo- | LwaCas13a | GATTTAGACTACCCCAAAA | ACCATA | GATTTAGACTAC | ther- | 2a |
| | nuclease | | ACGAAGGGGACTAAAACA | TTTCTC | CCCAAAAACGAA | mo- | |
| | valida- | | CCATATTTCTCTACACCTTT | TACACC | GGGGACTAAAAC | nu- | |
| | tion | | TTTAGGA | AGGA | (SEQ ID. | clease | |
| | LwaCas13 | | (SEQ ID. NO: 2703) | (SEQ ID. | NO: 2705) | | |
| | a 12 | | | NO: | | | |
| | | | | 2704) | | | |
| 2a | thermo- | LwaCas13a | GATTTAGACTACCCCAAAA | CTTTTT | GATTTAGACTAC | ther- | 2a |
| | nuclease | | ACGAAGGGGACTAAAACC | TAGGAT | CCCAAAAACGAA | mo- | |
| | valida- | | TTTTTTAGGATGCTTTGTTT | GCTTTG | GGGGACTAAAAC | nu- | |
| | tion | | CAGGTGT | TTTCAG | (SEQ ID. | clease | |
| | LwaCas13 | | (SEQ ID. NO: 2706) | GTGT | NO: 2708) | | |
| | a 13 | | | (SEQ ID. | | | |
| | | | | NO: | | | |
| | | | | 2707) | | | |
| 2a | thermo- | LwaCas13a | GATTTAGACTACCCCAAAA | TACACC | GATTTAGACTAC | ther- | 2a |
| | nuclease | | ACGAAGGGGACTAAAACT | AGGATG | CCCAAAAACGAA | mo- | |
| | valida- | | ACACCTTTTTTAGGATGCTT | CTTTGT | GGGGACTAAAAC | nu- | |
| | tion | | TGTTTCA | TTCA | (SEQ ID. | clease | |
| | LwaCas13 | | (SEQ ID. NO: 2709) | (SEQ ID. | NO: 2711) | | |
| | a 14 | | | NO: | | | |
| | | | | 2710) | | | |
| 2a | thermo- | LwaCas13a | GATTTAGACTACCCCAAAA | TCTTTT | GATTTAGACTAC | ther- | 2a |
| | nuclease | | ACGAAGGGGACTAAAACT | TCGTAA | CCCAAAAACGAA | mo- | |
| | valida- | | CTTTTTCGTAAATGCACTTG | ATGCAC | GGGGACTAAAAC | nu- | |
| | tion | | CTTCAGG | TTGCTT | (SEQ ID. | clease | |
| | LwaCas13 | | (SEQ ID. NO: 2712) | CAGG | NO: 2714) | | |
| | a 15 | | | (SEQ ID. | | | |
| | | | | NO: | | | |
| | | | | 2713) | | | |
| 2a | thermo- | LwaCas13a | GATTTAGACTACCCCAAAA | TTTTCT | GATTTAGACTAC | ther- | 2a |
| | nuclease | | ACGAAGGGGACTAAAACTT | TTGCAT | CCCAAAAACGAA | mo- | |
| | valida- | | TTCTTTGCATTTTCTACCAT | TTTCTA | GGGGACTAAAAC | nu- | |
| | tion | | CTTTTT | CCATCT | (SEQ ID. | clease | |
| | LwaCas13 | | (SEQ ID. NO: 2715) | TTTT | NO: 2717) | | |
| | a 16 | | | (SEQ ID. | | | |
| | | | | NO: | | | |
| | | | | 2716) | | | |
| 2a | thermo- | LwaCas13a | GATTTAGACTACCCCAAAA | TGAATG | GATTTAGACTAC | ther- | 2a |
| | nuclease | | ACGAAGGGGACTAAAACT | TCATTG | CCCAAAAACGAA | mo- | |
| | valida- | | GAATGTCATTGGTTGACCT | GTTGAC | GGGGACTAAAAC | nu- | |
| | tion | | TTGTACAT | CTTTGT | (SEQ ID. | clease | |
| | LwaCas13 | | (SEQ ID. NO: 2718) | ACAT | NO: 2720) | | |
| | a 17 | | | (SEQ ID. | | | |
| | | | | NO: | | | |
| | | | | 2719) | | | |
| 2a | thermo- | LwaCas13a | GATTTAGACTACCCCAAAA | AGGATG | GATTTAGACTAC | ther- | 2a |
| | nuclease | | ACGAAGGGGACTAAAACTT | CTTTGT | CCCAAAAACGAA | mo- | |
| | valida- | | TTTTAGGATGCTTTGTTTCA | TTCAGG | GGGGACTAAAAC | nu- | |
| | tion | | GGTGTA | TGTA | (SEQ ID. | clease | |
| | LwaCas13 | | (SEQ ID. NO: 2721) | (SEQ ID. | NO: 2723) | | |
| | a 18 | | | NO: | | | |
| | | | | 2722) | | | |
| 2a | thermo- | LwaCas13a | GATTTAGACTACCCCAAAA | TTTGTT | GATTTAGACTAC | ther- | 2a |
| | nuclease | | ACGAAGGGGACTAAAACTT | TCAGGT | CCCAAAAACGAA | mo- | |
| | valida- | | TGTTTCAGGTGTATCAACC | GTATCA | GGGGACTAAAAC | nu- | |
| | tion | | AATAATA | ACCAAT | (SEQ ID. | clease | |
| | LwaCas13 | | (SEQ ID. NO: 2724) | AATA | NO: 2726) | | |
| | a 19 | | | (SEQ ID. | | | |
| | | | | NO: | | | |
| | | | | 2725) | | | |
| 2a | thermo- | LwaCas13a | GATTTAGACTACCCCAAAA | TTGCTT | GATTTAGACTAC | ther- | 2a |
| | nuclease | | ACGAAGGGGACTAAAACTT | CAGGA | CCCAAAAACGAA | mo- | |
| | valida- | | GCTTCAGGACCATATTTCT | CCATAT | GGGGACTAAAAC | nu- | |
| | tion | | CTACACC | TTCTCT | (SEQ ID. | clease | |
| | LwaCas13 | | (SEQ ID. NO: 2727) | ACACC | NO: 2729) | | |
| | a 20 | | | (SEQ ID. | | | |
| | | | | NO: | | | |
| | | | | 2728) | | | |
| 2a | thermo- | LwaCas13a | GATTTAGACTACCCCAAAA | TCAGGT | GATTTAGACTAC | ther- | 2a |
| | nuclease | | ACGAAGGGGACTAAAACT | GTATCA | CCCAAAAACGAA | mo- | |
| | valida- | | CAGGTGTATCAACCAATAA | ACCAAT | GGGGACTAAAAC | nu- | |
| | tion | | TAGTCTGA | AATAGT | (SEQ ID. | clease | |
| | LwaCas13 | | (SEQ ID. NO: 2730) | CTGA | NO: 2732) | | |
| | a 21 | | | (SEQ ID. | | | |
| | | | | NO: | | | |
| | | | | 2731) | | | |
| 2a | thermo- | LwaCas13a | GATTTAGACTACCCCAAAA | ACTTGC | GATTTAGACTAC | ther- | 2a |
| | nuclease | | ACGAAGGGGACTAAAACA | TTCAGG | CCCAAAAACGAA | mo- | |
| | valida- | | CTTGCTTCAGGACCATATT | ACCATA | GGGGACTAAAAC | nu- | |
| | tion | | TCTCTACA | TTTCTC | (SEQ ID. | clease | |
| | LwaCas13 | | (SEQ ID. NO: 2733) | TACA | NO: 2735) | | |
| | a 22 | | | (SEQ ID. | | | |
| | | | | NO: | | | |
| | | | | 2734) | | | |
| 2a | thermo- | LwaCas13a | GATTTAGACTACCCCAAAA | TTTGTT | GATTTAGACTAC | ther- | 2a |
| | nuclease | | ACGAAGGGGACTAAAACTT | TCAGGT | CCCAAAAACGAA | mo- | |
| | valida- | | TGTTTCAGGTGTATCAACC | GTATCA | GGGGACTAAAAC | nu- | |
| | tion | | AATAATA | ACCAAT | (SEQ ID. | clease | |
| | LwaCas13 | | (SEQ ID. NO: 2736) | AATA | NO: 2738) | | |
| | a 23 | | | (SEQ ID. | | | |
| | | | | NO: | | | |
| | | | | 2737) | | | |
| 2a | thermo- | LwaCas13a | GATTTAGACTACCCCAAAA | TCTACA | GATTTAGACTAC | ther- | 2a |
| | nuclease | | ACGAAGGGGACTAAAACT | CCTTTT | CCCAAAAACGAA | mo- | |
| | valida- | | CTACACCTTTTTTAGGATG | TTAGGA | GGGGACTAAAAC | nu- | |
| | tion | | CTTTGTTT | TGCTTT | (SEQ ID. | clease | |
| | LwaCas13 | | (SEQ ID. NO: 2739) | GTTT | NO: 2741) | | |
| | a 24 | | | (SEQ ID. | | | |
| | | | | NO: | | | |
| | | | | 2740) | | | |
| 2a | thermo- | LwaCas13a | GATTTAGACTACCCCAAAA | CTTCAG | GATTTAGACTAC | ther- | 2a |
| | nuclease | | ACGAAGGGGACTAAAACC | GACCAT | CCCAAAAACGAA | mo- | |
| | valida- | | TTCAGGACCATATTTCTCT | ATTTCT | GGGGACTAAAAC | nu- | |
| | tion | | ACACCTTT | CTACAC | (SEQ ID. | clease | |
| | LwaCas13 | | (SEQ ID. NO: 2742) | CTTT | NO: 2744) | | |
| | a 25 | | | (SEQ ID. | | | |
| | | | | NO: | | | |
| | | | | 2743) | | | |
| 2a | thermo- | LwaCas13a | GATTTAGACTACCCCAAAA | TGACCT | GATTTAGACTAC | ther- | 2a |
| | nuclease | | ACGAAGGGGACTAAAACT | TTGTAC | CCCAAAAACGAA | mo- | |
| | valida- | | GACCTTTGTACATTAATTT | ATTAAT | GGGGACTAAAAC | nu- | |
| | tion | | AACAGTAT | TTAACA | (SEQ ID. | clease | |
| | LwaCas13 | | (SEQ ID. NO: 2745) | GTAT | NO: 2747) | | |
| | a 26 | | | (SEQ ID. | | | |
| | | | | NO: | | | |
| | | | | 2746) | | | |
| 2a | thermo- | LwaCas13a | GATTTAGACTACCCCAAAA | ATTGGT | GATTTAGACTAC | ther- | 2a |
| | nuclease | | ACGAAGGGGACTAAAACA | TGACCT | CCCAAAAACGAA | mo- | |
| | valida- | | TTGGTTGACCTTTGTACATT | TTGTAC | GGGGACTAAAAC | nu- | |
| | tion | | AATTTAA | ATTAAT | (SEQ ID. | clease | |
| | LwaCas13 | | (SEQ ID. NO: 2748) | TTAA | NO: 2750) | | |
| | a 27 | | | (SEQ ID. | | | |
| | | | | NO: | | | |
| | | | | 2749) | | | |
| 2a | thermo- | LwaCas13a | GATTTAGACTACCCCAAAA | GTCATT | GATTTAGACTAC | ther- | 2a |
| | nuclease | | ACGAAGGGGACTAAAACG | GGTTGA | CCCAAAAACGAA | mo- | |
| | valida- | | TCATTGGTTGACCTTTGTAC | CCTTTG | GGGGACTAAAAC | nu- | |
| | tion | | ATTAATT | TACATT | (SEQ ID. | clease | |
| | LwaCas13 | | (SEQ ID. NO: 2751) | AATT | NO: 2753) | | |
| | a 28 | | | (SEQ ID. | | | |
| | | | | NO: | | | |
| | | | | 2752) | | | |
| 2a | thermo- | LwaCas13a | GATTTAGACTACCCCAAAA | TTCTCT | GATTTAGACTAC | ther- | 2a |
| | nuclease | | ACGAAGGGGACTAAAACTT | ACACCT | CCCAAAAACGAA | mo- | |
| | valida- | | CTCTACACCTTTTTTAGGAT | TTTTTA | GGGGACTAAAAC | nu- | |
| | tion | | GCTTTG | GGATGC | (SEQ ID. | clease | |
| | LwaCas13 | | (SEQ ID. NO: 2754) | TTTG | NO: 2756) | | |
| | a 29 | | | (SEQ ID. | | | |
| | | | | NO: | | | |
| | | | | 2755) | | | |
| 2a | thermo- | LwaCas13a | GATTTAGACTACCCCAAAA | GCATTT | GATTTAGACTAC | ther- | 2a |
| | nuclease | | ACGAAGGGGACTAAAACG | TCTACC | CCCAAAAACGAA | mo- | |
| | valida- | | CATTTTCTACCATCTTTTTC | ATCTTT | GGGGACTAAAAC | nu- | |
| | tion | | GTAAATG | TTCGTA | (SEQ ID. | clease | |
| | LwaCas13 | | (SEQ ID. NO: 2757) | AATG | NO: 2759) | | |
| | a 30 | | | (SEQ ID. | | | |
| | | | | NO: | | | |
| | | | | 2758) | | | |
| 2a | APML | LwaCas13a | GATTTAGACTACCCCAAAA | GCGCCA | GATTTAGACTAC | APML | 2a |
| | long | | ACGAAGGGGACTAAAACG | CTGGCC | CCCAAAAACGAA | long | |
| | valida- | | CGCCACTGGCCACGTGGTT | ACGTGG | GGGGACTAAAAC | | |
| | tion | | GCTGTTGG | TTGCTG | (SEQ ID. | | |
| | LwaCas13 | | (SEQ ID. NO: 2760) | TTGG | NO: 2762) | | |
| | a 1 | | | (SEQ ID. | | | |
| | | | | NO: | | | |
| | | | | 2761) | | | |
| 2a | APML | LwaCas13a | GATTTAGACTACCCCAAAA | TGGCTG | GATTTAGACTAC | APML | 2a |
| | long | | ACGAAGGGGACTAAAACT | CCTCCC | CCCAAAAACGAA | long | |
| | valida- | | GGCTGCCTCCCCGGCGCCA | CGGCGC | GGGGACTAAAAC | | |
| | tion | | CTGGCCAC | CACTGG | (SEQ ID. | | |
| | LwaCas13 | | (SEQ ID. NO: 2763) | CCAC | NO: 2765) | | |
| | a 2 | | | (SEQ ID. | | | |
| | | | | NO: | | | |
| | | | | 2764) | | | |
| 2a | APML | LwaCas13a | GATTTAGACTACCCCAAAA | CTGCCT | GATTTAGACTAC | APML | 2a |
| | long | | ACGAAGGGGACTAAAACG | CCCCGG | CCCAAAAACGAA | long | |
| | valida- | | TGCCTCCCCGGCGCCACTG | CGCCAC | GGGGACTAAAAC | | |
| | tion | | GCCACGTG | TGGCCA | (SEQ ID. | | |
| | LwaCas13 | | (SEQ ID. NO: 2766) | CGTG | NO: 2768) | | |
| | a 3 | | | (SEQ ID. | | | |
| | | | | NO: | | | |
| | | | | 2767) | | | |
| 2a | APML | LwaCas13a | GATTTAGACTACCCCAAAA | GGCTGC | GATTTAGACTAC | APML | 2a |
| | long | | ACGAAGGGGACTAAAACG | CTCCCC | CCCAAAAACGAA | long | |
| | valida- | | GCTGCCTCCCCGGCGCCAC | GGCGCC | GGGGACTAAAAC | | |
| | tion | | TGGCCACG | ACTGGC | (SEQ ID. | | |
| | LwaCas13 | | (SEQ ID. NO: 2769) | CACG | NO: 2771) | | |
| | a 4 | | | (SEQ ID. | | | |
| | | | | NO: | | | |
| | | | | 2770) | | | |
| 2a | APML | LwaCas13a | GATTTAGACTACCCCAAAA | CCCCGG | GATTTAGACTAC | APML | 2a |
| | long | | ACGAAGGGGACTAAAACG | CGCCAC | CCCAAAAACGAA | long | |
| | valida- | | CCCGGCGCCACTGGCCACG | TGGCCA | GGGGACTAAAAC | | |
| | tion | | TGGTTGCT | CGTGGT | (SEQ ID. | | |
| | LwaCas13 | | (SEQ ID. NO: 2772) | TGCT | NO: 2774) | | |
| | a 5 | | | (SEQ ID. | | | |
| | | | | NO: | | | |
| | | | | 2773) | | | |
| 2a | APML | LwaCas13a | GATTTAGACTACCCCAAAA | GCTGCC | GATTTAGACTAC | APML | 2a |
| | long | | ACGAAGGGGACTAAAACG | TCCCCG | CCCAAAAACGAA | long | |
| | valida- | | CTGCCTCCCCGGCGCCACT | GCGCCA | GGGGACTAAAAC | | |
| | tion | | GGCCACGT | CTGGCC | (SEQ ID. | | |
| | LwaCas13 | | (SEQ ID. NO: 2775) | ACGT | NO: 2777) | | |
| | a 6 | | | (SEQ ID. | | | |
| | | | | NO: | | | |
| | | | | 2776) | | | |
| 2a | APML | LwaCas13a | GATTTAGACTACCCCAAAA | CGCCAC | GATTTAGACTAC | APML | 2a |
| | long | | ACGAAGGGGACTAAAACC | TGGCCA | CCCAAAAACGAA | long | |
| | valida- | | GCCACTGGCCACGTGGTTG | CGTGGT | GGGGACTAAAAC | | |
| | tion | | CTGTTGGG | TGCTGT | (SEQ ID. | | |
| | LwaCas13 | | (SEQ ID. NO: 2778) | TGGG | NO: 2780) | | |
| | a 7 | | | (SEQ ID. | | | |
| | | | | NO: | | | |
| | | | | 2779) | | | |
| 2a | APML | LwaCas13a | GATTTAGACTACCCCAAAA | CGGCGC | GATTTAGACTAC | APML | 2a |
| | long | | ACGAAGGGGACTAAAACC | CACTGG | CCCAAAAACGAA | long | |
| | valida- | | GGCGCCACTGGCCACGTGG | CCACGT | GGGGACTAAAAC | | |
| | tion | | TTGCTGTT | GGTTGC | (SEQ ID. | | |
| | LwaCas13 | | (SEQ ID. NO: 2781) | TGTT | NO: 2783) | | |
| | a 8 | | | (SEQ ID. | | | |
| | | | | NO: | | | |
| | | | | 2782) | | | |
| 2a | APML | LwaCas13a | GATTTAGACTACCCCAAAA | ATGGCT | GATTTAGACTAC | APML | 2a |
| | long | | ACGAAGGGGACTAAAACA | GCCTCC | CCCAAAAACGAA | long | |
| | valida- | | TGGCTGCCTCCCCGGCGCC | CCGGCG | GGGGACTAAAAC | | |
| | tion | | ACTGGCCA | CCACTG | (SEQ ID. | | |
| | LwaCas13 | | (SEQ ID. NO: 2784) | GCCA | NO: 2786) | | |
| | a 9 | | | (SEQ ID. | | | |
| | | | | NO: | | | |
| | | | | 2785) | | | |
| 2a | APML | LwaCas13a | GATTTAGACTACCCCAAAA | CCCGGC | GATTTAGACTAC | APML | 2a |
| | long | | ACGAAGGGGACTAAAACC | GCCACT | CCCAAAAACGAA | long | |
| | valida- | | CCGGCGCCACTGGCCACGT | GGCCAC | GGGGACTAAAAC | | |
| | tion | | GGTTGCTG | GTGGTT | (SEQ ID. | | |
| | LwaCas13 | | (SEQ ID. NO: 2787) | GCTG | NO: 2789) | | |
| | a 10 | | | (SEQ ID. | | | |
| | | | | NO: | | | |
| | | | | 2788) | | | |
| 2a | APML | LwaCas13a | GATTTAGACTACCCCAAAA | AATGGC | GATTTAGACTAC | APML | 2a |
| | long | | ACGAAGGGGACTAAAACA | TGCCTC | CCCAAAAACGAA | long | |
| | valida- | | ATGGCTGCCTCCCCGGCGC | CCCGGC | GGGGACTAAAAC | | |
| | tion | | CACTGGCC | GCCACT | (SEQ ID. | | |
| | LwaCas13 | | (SEQ ID. NO: 2790) | GGCC | NO: 2792) | | |
| | a 11 | | | (SEQ ID. | | | |
| | | | | NO: | | | |
| | | | | 2791) | | | |
| 2a | APML | LwaCas13a | GATTTAGACTACCCCAAAA | CTCCCC | GATTTAGACTAC | APML | 2a |
| | long | | ACGAAGGGGACTAAAACC | GGCGCC | CCCAAAAACGAA | long | |
| | valida- | | TCCCCGGCGCCACTGGCCA | ACTGGC | GGGGACTAAAAC | | |
| | tion | | CGTGGTTG | CACGTG | (SEQ ID. | | |
| | LwaCas13 | | (SEQ ID. NO: 2793) | GTTG | NO: 2795) | | |
| | a 12 | | | (SEQ ID. | | | |
| | | | | NO: | | | |
| | | | | 2794) | | | |
| 2a | APML | LwaCas13a | GATTTAGACTACCCCAAAA | CCTCCC | GATTTAGACTAC | APML | 2a |
| | long | | ACGAAGGGGACTAAAACC | CGGCGC | CCCAAAAACGAA | long | |
| | valida- | | CTCCCCGGCGCCACTGGCC | CACTGG | GGGGACTAAAAC | | |
| | tion | | ACGTGGTT | CCACGT | (SEQ ID. | | |
| | LwaCas13 | | (SEQ ID. NO: 2796) | GGTT | NO: 2798) | | |
| | a 13 | | | (SEQ ID. | | | |
| | | | | NO: | | | |
| | | | | 2797) | | | |
| 2a | APML | LwaCas13a | GATTTAGACTACCCCAAAA | TCAATG | GATTTAGACTAC | APML | 2a |
| | long | | ACGAAGGGGACTAAAACT | GCTGCC | CCCAAAAACGAA | long | |
| | valida- | | CAATGGCTGCCTCCCCGGC | TCCCCG | GGGGACTAAAAC | | |
| | tion | | GCCACTGG | GCGCCA | (SEQ ID. | | |
| | LwaCas13 | | (SEQ ID. NO: 2799) | CTGG | NO: 2801) | | |
| | a 14 | | | (SEQ ID. | | | |
| | | | | NO: | | | |
| | | | | 2800) | | | |
| 2a | APML | LwaCas13a | GATTTAGACTACCCCAAAA | CCGGCG | GATTTAGACTAC | APML | 2a |
| | long | | ACGAAGGGGACTAAAACC | CCACTG | CCCAAAAACGAA | long | |
| | valida- | | CGGCGCCACTGGCCACGTG | GCCACG | GGGGACTAAAAC | | |
| | tion | | GTTGCTGT | TGGTTG | (SEQ ID. | | |
| | LwaCas13 | | (SEQ ID. NO: 2802) | CTGT | NO: 2804) | | |
| | a 15 | | | (SEQ ID. | | | |
| | | | | NO: | | | |
| | | | | 2803) | | | |
| 2a | APML | LwaCas13a | GATTTAGACTACCCCAAAA | CAATGG | GATTTAGACTAC | APML | 2a |
| | long | | ACGAAGGGGACTAAAACC | CTGCCT | CCCAAAAACGAA | long | |
| | valida- | | AATGGCTGCCTCCCCGGCG | CCCCGG | GGGGACTAAAAC | | |
| | tion | | CCACTGGC | CGCCAC | (SEQ ID. | | |
| | LwaCas13 | | (SEQ ID. NO: 2805) | TGGC | NO: 2807) | | |
| | a 16 | | | (SEQ ID. | | | |
| | | | | NO: | | | |
| | | | | 2806) | | | |
| 2a | APML | LwaCas13a | GATTTAGACTACCCCAAAA | TGCCTC | GATTTAGACTAC | APML | 2a |
| | long | | ACGAAGGGGACTAAAACT | CCCGGC | CCCAAAAACGAA | long | |
| | valida- | | GCCTCCCCGGCGCCACTGG | GCCACT | GGGGACTAAAAC | | |
| | tion | | CCACGTGG | GGCCAC | (SEQ ID. | | |
| | LwaCas13 | | (SEQ ID. NO: 2808) | GTGG | NO: 2810) | | |
| | a 17 | | | (SEQ ID. | | | |
| | | | | NO: | | | |
| | | | | 2809) | | | |
| 2a | APML | LwaCas13a | GATTTAGACTACCCCAAAA | TCCCCG | GATTTAGACTAC | APML | 2a |
| | long | | ACGAAGGGGACTAAAACT | GCGCCA | CCCAAAAACGAA | long | |
| | valida- | | CCCCGGCGCCACTGGCCAC | CTGGCC | GGGGACTAAAAC | | |
| | tion | | GTGGTTGC | ACGTGG | (SEQ ID. | | |
| | LwaCas13 | | (SEQ ID. NO: 2811) | TTGC | NO: 2813) | | |
| | a 18 | | | (SEQ ID. | | | |
| | | | | NO: | | | |
| | | | | 2812) | | | |
| 2a | APML | LwaCas13a | GATTTAGACTACCCCAAAA | GGCGCC | GATTTAGACTAC | APML | 2a |
| | long | | ACGAAGGGGACTAAAACG | ACTGGC | CCCAAAAACGAA | long | |
| | valida- | | GCGCCACTGGCCACGTGGT | CACGTG | GGGGACTAAAAC | | |
| | tion | | TGCTGTTG | GTTGCT | (SEQ ID. | | |
| | LwaCas13 | | (SEQ ID. NO: 2814) | GTTG | NO: 2816) | | |
| | a 19 | | | (SEQ ID. | | | |
| | | | | NO: | | | |
| | | | | 2815) | | | |
| 2a | APML | LwaCas13a | GATTTAGACTACCCCAAAA | GCCTCC | GATTTAGACTAC | APML | 2a |
| | long | | ACGAAGGGGACTAAAACG | CCGGCG | CCCAAAAACGAA | long | |
| | valida- | | CCTCCCCGGCGCCACTGGC | CCACTG | GGGGACTAAAAC | | |
| | tion | | CACGTGGT | GCCACG | (SEQ ID. | | |
| | LwaCas13 | | (SEQ ID. NO: 2817) | TGGT | NO: 2819) | | |
| | a 20 | | | (SEQ ID. | | | |
| | | | | NO: | | | |
| | | | | 2818) | | | |
| 2a | APML | LwaCas13a | GATTTAGACTACCCCAAAA | TCTCAA | GATTTAGACTAC | APML | 2a |
| | short | | ACGAAGGGGACTAAAACT | TGGCTT | CCCAAAAACGAA | short | |
| | valida- | | CTCAATGGCTTTCCCCTGG | TCCCCT | GGGGACTAAAAC | | |
| | tion | | GTGATGCA | GGGTGA | (SEQ ID. | | |
| | LwaCas13 | | (SEQ ID. NO: 2820) | TGCA | NO: 2822) | | |
| | a 1 | | | (SEQ ID. | | | |
| | | | | NO: | | | |
| | | | | 2821) | | | |
| 2a | APML | LwaCas13a | GATTTAGACTACCCCAAAA | ATGGCT | GATTTAGACTAC | APML | 2a |
| | short | | ACGAAGGGGACTAAAACA | TTCCCC | CCCAAAAACGAA | short | |
| | valida- | | TGGCTTTCCCCTGGGTGAT | TGGGTG | GGGGACTAAAAC | | |
| | tion | | GCAAGAGC | ATGCAA | (SEQ ID. | | |
| | LwaCas13 | | (SEQ ID. NO: 2823) | GAGC | NO: 2825) | | |
| | a 2 | | | (SEQ ID. | | | |
| | | | | NO: | | | |
| | | | | 2824) | | | |
| 2a | APML | LwaCas13a | GATTTAGACTACCCCAAAA | AATGGC | GATTTAGACTAC | APML | 2a |
| | short | | ACGAAGGGGACTAAAACA | TTTCCC | CCCAAAAACGAA | short | |
| | valida- | | ATGGCTTTCCCCTGGGTGA | CTGGGT | GGGGACTAAAAC | | |
| | tion | | TGCAAGAG | GATGCA | (SEQ ID. | | |
| | LwaCas13 | | (SEQ ID. NO: 2826) | AGAG | NO: 2828) | | |
| | a 3 | | | (SEQ ID. | | | |
| | | | | NO: | | | |
| | | | | 2827) | | | |
| 2a | APML | LwaCas13a | GATTTAGACTACCCCAAAA | GGGTGA | GATTTAGACTAC | APML | 2a |
| | short | | ACGAAGGGGACTAAAACG | TGCAAG | CCCAAAAACGAA | short | |
| | valida- | | GGTGATGCAAGAGCTGAG | AGCTGA | GGGGACTAAAAC | | |
| | tion | | GTCCTGCAG | GGTCCT | (SEQ ID. | | |
| | LwaCas13 | | (SEQ ID. NO: 2829) | GCAG | NO: 2831) | | |
| | a 4 | | | (SEQ ID. | | | |
| | | | | NO: | | | |
| | | | | 2830) | | | |
| 2a | APML | LwaCas13a | GATTTAGACTACCCCAAAA | TGGCTT | GATTTAGACTAC | APML | 2a |
| | short | | ACGAAGGGGACTAAAACT | TCCCCT | CCCAAAAACGAA | short | |
| | valida- | | GGCTTTCCCCTGGGTGATG | GGGTGA | GGGGACTAAAAC | | |
| | tion | | CAAGAGCT | TGCAAG | (SEQ ID. | | |
| | LwaCas13 | | (SEQ ID. NO: 2832) | AGCT | NO: 2834) | | |
| | a 5 | | | (SEQ ID. | | | |
| | | | | NO: | | | |
| | | | | 2833) | | | |
| 2a | APML | LwaCas13a | GATTTAGACTACCCCAAAA | CTCAAT | GATTTAGACTAC | APML | 2a |
| | short | | ACGAAGGGGACTAAAACG | GGCTTT | CCCAAAAACGAA | short | |
| | valida- | | TCAATGGCTTTCCCCTGGG | CCCCTG | GGGGACTAAAAC | | |
| | tion | | TGATGCAA | GGTGAT | (SEQ ID. | | |
| | LwaCas13 | | (SEQ ID. NO: 2835) | GCAA | NO: 2837) | | |
| | a 6 | | | (SEQ ID. | | | |
| | | | | NO: | | | |
| | | | | 2836) | | | |
| 2a | APML | LwaCas13a | GATTTAGACTACCCCAAAA | TTCCCC | GATTTAGACTAC | APML | 2a |
| | short | | ACGAAGGGGACTAAAACTT | TGGGTG | CCCAAAAACGAA | short | |
| | valida- | | CCCCTGGGTGATGCAAGAG | ATGCAA | GGGGACTAAAAC | | |
| | tion | | CTGAGGT | GAGCTG | (SEQ ID. | | |
| | LwaCas13 | | (SEQ ID. NO: 2838) | AGGT | NO: 2840) | | |
| | a 7 | | | (SEQ ID. | | | |
| | | | | NO: | | | |
| | | | | 2839) | | | |
| 2a | APML | LwaCas13a | GATTTAGACTACCCCAAAA | GCTTTC | GATTTAGACTAC | APML | 2a |
| | short | | ACGAAGGGGACTAAAACG | CCCTGG | CCCAAAAACGAA | short | |
| | valida- | | CTTTCCCCTGGGTGATGCA | GTGATG | GGGGACTAAAAC | | |
| | tion | | AGAGCTGA | CAAGA | (SEQ ID. | | |
| | LwaCas13 | | (SEQ ID. NO: 2841) | GCTGA | NO: 2843) | | |
| | a 8 | | | (SEQ ID. | | | |
| | | | | NO: | | | |
| | | | | 2842) | | | |
| 2a | APML | LwaCas13a | GATTTAGACTACCCCAAAA | TCCCCT | GATTTAGACTAC | APML | 2a |
| | short | | ACGAAGGGGACTAAAACT | GGGTGA | CCCAAAAACGAA | short | |
| | valida- | | CCCCTGGGTGATGCAAGAG | TGCAAG | GGGGACTAAAAC | | |
| | tion | | CTGAGGTC | AGCTGA | (SEQ ID. | | |
| | LwaCas13 | | (SEQ ID. NO: 2844) | GGTC | NO: 2846) | | |
| | a 9 | | | (SEQ ID. | | | |
| | | | | NO: | | | |
| | | | | 2845) | | | |
| 2a | APML | LwaCas13a | GATTTAGACTACCCCAAAA | CTTTCC | GATTTAGACTAC | APML | 2a |
| | short | | ACGAAGGGGACTAAAACC | CCTGGG | CCCAAAAACGAA | short | |
| | valida- | | TTTCCCCTGGGTGATGCAA | TGATGC | GGGGACTAAAAC | | |
| | tion | | GAGCTGAG | AAGAG | (SEQ ID. | | |
| | LwaCas13 | | (SEQ ID. NO: 2847) | CTGAG | NO: 2849) | | |
| | a 10 | | | (SEQ ID. | | | |
| | | | | NO: | | | |
| | | | | 2848) | | | |
| 2a | APML | LwaCas13a | GATTTAGACTACCCCAAAA | CAATGG | GATTTAGACTAC | APML | 2a |
| | short | | ACGAAGGGGACTAAAACC | CTTTCC | CCCAAAAACGAA | short | |
| | valida- | | AATGGCTTTCCCCTGGGTG | CCTGGG | GGGGACTAAAAC | | |
| | tion | | ATGCAAGA | TGATGC | (SEQ ID. | | |
| | LwaCas13 | | (SEQ ID. NO: 2850) | AAGA | NO: 2852) | | |
| | a 11 | | | (SEQ ID. | | | |
| | | | | NO: | | | |
| | | | | 2851) | | | |
| 2a | APML | LwaCas13a | GATTTAGACTACCCCAAAA | CCTGGG | GATTTAGACTAC | APML | 2a |
| | short | | ACGAAGGGGACTAAAACC | TGATGC | CCCAAAAACGAA | short | |
| | valida- | | CTGGGTGATGCAAGAGCTG | AAGAG | GGGGACTAAAAC | | |
| | tion | | AGGTCCTG | CTGAGG | (SEQ ID. | | |
| | LwaCas13 | | (SEQ ID. NO: 2853) | TCCTG | NO: 2855) | | |
| | a 12 | | | (SEQ ID. | | | |
| | | | | NO: | | | |
| | | | | 2854) | | | |
| 2a | APML | LwaCas13a | GATTTAGACTACCCCAAAA | GGTCTC | GATTTAGACTAC | APML | 2a |
| | short | | ACGAAGGGGACTAAAACG | AATGGC | CCCAAAAACGAA | short | |
| | valida- | | GTCTCAATGGCTTTCCCCT | TTTCCC | GGGGACTAAAAC | | |
| | tion | | GGGTGATG | CTGGGT | (SEQ ID. | | |
| | LwaCas13 | | (SEQ ID. NO: 2856) | GATG | NO: 2858) | | |
| | a 13 | | | (SEQ ID. | | | |
| | | | | NO: | | | |
| | | | | 2857) | | | |
| 2a | APML | LwaCas13a | GATTTAGACTACCCCAAAA | GGGTCT | GATTTAGACTAC | APML | 2a |
| | short | | ACGAAGGGGACTAAAACG | CAATGG | CCCAAAAACGAA | short | |
| | valida- | | GGTCTCAATGGCTTTCCCC | CTTTCC | GGGGACTAAAAC | | |
| | tion | | TGGGTGAT | CCTGGG | (SEQ ID. | | |
| | LwaCas13 | | (SEQ ID. NO: 2859) | TGAT | NO: 2861) | | |
| | a 14 | | | (SEQ ID. | | | |
| | | | | NO: | | | |
| | | | | 2860) | | | |
| 2a | APML | LwaCas13a | GATTTAGACTACCCCAAAA | GGCTTT | GATTTAGACTAC | APML | 2a |
| | short | | ACGAAGGGGACTAAAACG | CCCCTG | CCCAAAAACGAA | short | |
| | valida- | | GCTTTCCCCTGGGTGATGC | GGTGAT | GGGGACTAAAAC | | |
| | tion | | AAGAGCTG | GCAAG | (SEQ ID. | | |
| | LwaCas13 | | (SEQ ID. NO: 2862) | AGCTG | NO: 2864) | | |
| | a 15 | | | (SEQ ID. | | | |
| | | | | NO: | | | |
| | | | | 2863) | | | |
| 2a | APML | LwaCas13a | GATTTAGACTACCCCAAAA | TTTCCC | GATTTAGACTAC | APML | 2a |
| | short | | ACGAAGGGGACTAAAACTT | CTGGGT | CCCAAAAACGAA | short | |
| | valida- | | TCCCCTGGGTGATGCAAGA | GATGCA | GGGGACTAAAAC | | |
| | tion | | GCTGAGG | AGAGCT | (SEQ ID. | | |
| | LwaCas13 | | (SEQ ID. NO: 2865) | GAGG | NO: 2867) | | |
| | a 16 | | | (SEQ ID. | | | |
| | | | | NO: | | | |
| | | | | 2866) | | | |
| 2a | APML | LwaCas13a | GATTTAGACTACCCCAAAA | CCCCTG | GATTTAGACTAC | APML | 2a |
| | short | | ACGAAGGGGACTAAAACG | GGTGAT | CCCAAAAACGAA | short | |
| | valida- | | CCCTGGGTGATGCAAGAGC | GCAAG | GGGGACTAAAAC | | |
| | tion | | TGAGGTCC | AGCTGA | (SEQ ID. | | |
| | LwaCas13 | | (SEQ ID. NO: 2868) | GGTCC | NO: 2870) | | |
| | a 17 | | | (SEQ ID. | | | |
| | | | | NO: | | | |
| | | | | 2869) | | | |
| 2a | APML | LwaCas13a | GATTTAGACTACCCCAAAA | TGGGTG | GATTTAGACTAC | APML | 2a |
| | short | | ACGAAGGGGACTAAAACT | ATGCAA | CCCAAAAACGAA | short | |
| | valida- | | GGGTGATGCAAGAGCTGA | GAGCTG | GGGGACTAAAAC | | |
| | tion | | GGTCCTGCA | AGGTCC | (SEQ ID. | | |
| | LwaCas13 | | (SEQ ID. NO: 2871) | TGCA | NO: 2873) | | |
| | a 18 | | | (SEQ ID. | | | |
| | | | | NO: | | | |
| | | | | 2872) | | | |
| 2a | APML | LwaCas13a | GATTTAGACTACCCCAAAA | GTCTCA | GATTTAGACTAC | APML | 2a |
| | short | | ACGAAGGGGACTAAAACG | ATGGCT | CCCAAAAACGAA | short | |
| | valida- | | TCTCAATGGCTTTCCCCTG | TTCCCC | GGGGACTAAAAC | | |
| | tion | | GGTGATGC | TGGGTG | (SEQ ID. | | |
| | LwaCas13 | | (SEQ ID. NO: 2874) | ATGC | NO: 2876) | | |
| | a 19 | | | (SEQ ID. | | | |
| | | | | NO: | | | |
| | | | | 2875) | | | |
| 2a | APML | LwaCas13a | GATTTAGACTACCCCAAAA | CTGGGT | GATTTAGACTAC | APML | 2a |
| | short | | ACGAAGGGGACTAAAACC | GATGCA | CCCAAAAACGAA | short | |
| | valida- | | TGGGTGATGCAAGAGCTGA | AGAGCT | GGGGACTAAAAC | | |
| | tion | | GGTCCTGC | GAGGTC | (SEQ ID. | | |
| | LwaCas13 | | (SEQ ID. NO: 2877) | CTGC | NO: 2879) | | |
| | a 20 | | | (SEQ ID. | | | |
| | | | | NO: | | | |
| | | | | 2878) | | | |
| 2a | APML | LwaCas13a | GATTTAGACTACCCCAAAA | CCCTGG | GATTTAGACTAC | APML | 2a |
| | short | | ACGAAGGGGACTAAAACC | GTGATG | CCCAAAAACGAA | short | |
| | valida- | | CCTGGGTGATGCAAGAGCT | CAAGA | GGGGACTAAAAC | | |
| | tion | | GAGGTCCT | GCTGAG | (SEQ ID. | | |
| | LwaCas13 | | (SEQ ID. NO: 2880) | GTCCT | NO: 2882) | | |
| | a 21 | | | (SEQ ID. | | | |
| | | | | NO: | | | |
| | | | | 2881) | | | |
| 2a | APML | LwaCas13a | GATTTAGACTACCCCAAAA | TCAATG | GATTTAGACTAC | APML | 2a |
| | short | | ACGAAGGGGACTAAAACT | GCTTTC | CCCAAAAACGAA | short | |
| | valida- | | CAATGGCTTTCCCCTGGGT | CCCTGG | GGGGACTAAAAC | | |
| | tion | | GATGCAAG | GTGATG | (SEQ ID. | | |
| | LwaCas13 | | (SEQ ID. NO: 2883) | CAAG | NO: 2885) | | |
| | a 22 | | | (SEQ ID. | | | |
| | | | | NO: | | | |
| | | | | 2884) | | | |
| 2a | APML | LwaCas13a | GATTTAGACTACCCCAAAA | TGGGTC | GATTTAGACTAC | APML | 2a |
| | short | | ACGAAGGGGACTAAAACT | TCAATG | CCCAAAAACGAA | short | |
| | valida- | | GGGTCTCAATGGCTTTCCC | GCTTTC | GGGGACTAAAAC | | |
| | tion | | CTGGGTGA | CCCTGG | (SEQ ID. | | |
| | LwaCas13 | | (SEQ ID. NO: 2886) | GTGA | NO: 2888) | | |
| | a 23 | | | (SEQ ID. | | | |
| | | | | NO: | | | |
| | | | | 2887) | | | |
| 2b | APML | CcaCas13b | cggcgccactggccacgtggttgct | cggcgccac | GTTGGAACTGCT | APML | 2b |
| | long | | gttggGTTGGAACTGCTCTCATTTT | tggccacgt | CTCATTTTGGAGG | long | |
| | valida- | | GGAGGGTAATCACAAC | ggttgctgtt | GTAATCACAAC | | |
| | tion | | (SEQ ID. NO: 2889) | gg (SEQ | (SEQ ID. | | |
| | CcaCas13 | | | ID. NO: | NO: 2891) | | |
| | b 1 | | | 2890) | | | |
| 2b | APML | CcaCas13b | ccggcgccactggccacgtggttgc | ccggcgcca | GTTGGAACTGCT | APML | 2b |
| | long | | tgttgGTTGGAACTGCTCTCATTTT | ctggccacg | CTCATTTTGGAGG | long | |
| | valida- | | GGAGGGTAATCACAAC | tggttgctgtt | GTAATCACAAC | | |
| | tion | | (SEQ ID. NO: 2892) | g (SEQ | (SEQ ID. | | |
| | CcaCas13 | | | ID. NO: | NO: 2894) | | |
| | b 2 | | | 2893) | | | |
| 2b | APML | CcaCas13b | cccggcgccactggccacgtggttg | cccggcgcc | GTTGGAACTGCT | APML | 2b |
| | long | | ctgttGTTGGAACTGCTCTCATTTT | actggccac | CTCATTTTGGAGG | long | |
| | valida- | | GGAGGGTAATCACAAC | gtggttgctg | GTAATCACAAC | | |
| | tion | | (SEQ ID. NO: 2895) | tt (SEQ | (SEQ ID. | | |
| | CcaCas13 | | | ID. NO: | NO: 2897) | | |
| | b 3 | | | 2896) | | | |
| 2b | APML | CcaCas13b | ccccggcgccactggccacgtggtt | ccccggcgc | GTTGGAACTGCT | APML | 2b |
| | long | | gctgtGTTGGAACTGCTCTCATTTT | cactggcca | CTCATTTTGGAGG | long | |
| | valida- | | GGAGGGTAATCACAAC | cgtggttgct | GTAATCACAAC | | |
| | tion | | (SEQ ID. NO: 2898) | gt (SEQ | (SEQ ID. | | |
| | CcaCas13 | | | ID. NO: | NO: 2900) | | |
| | b 4 | | | 2899) | | | |
| 2b | APML | CcaCas13b | tccccggcgccactggccacgtggt | tccccggcg | GTTGGAACTGCT | APML | 2b |
| | long | | tgctgGTTGGAACTGCTCTCATTTT | ccactggcc | CTCATTTTGGAGG | long | |
| | valida- | | GGAGGGTAATCACAAC | acgtggttgc | GTAATCACAAC | | |
| | tion | | (SEQ ID. NO: 2901) | tg (SEQ | (SEQ ID. | | |
| | CcaCas13 | | | ID. NO: | NO: 2903) | | |
| | b 5 | | | 2902) | | | |
| 2b | APML | CcaCas13b | ctccccggcgccactggccacgtgg | ctccccggc | GTTGGAACTGCT | APML | 2b |
| | long | | ttgctGTTGGAACTGCTCTCATTTT | gccactggc | CTCATTTTGGAGG | long | |
| | valida- | | GGAGGGTAATCACAAC | cacgtggttg | GTAATCACAAC | | |
| | tion | | (SEQ ID. NO: 2904) | ct (SEQ | (SEQ ID. | | |
| | CcaCas13 | | | ID. NO: | NO: 2906) | | |
| | b 6 | | | 2905) | | | |
| 2b | APML | CcaCas13b | cctccccggcgccactggccacgtg | cctccccgg | GTTGGAACTGCT | APML | 2b |
| | long | | gttgcGTTGGAACTGCTCTCATTTT | cgccactgg | CTCATTTTGGAGG | long | |
| | valida- | | GGAGGGTAATCACAAC | ccacgtggtt | GTAATCACAAC | | |
| | tion | | (SEQ ID. NO: 2907) | gc (SEQ | (SEQ ID. | | |
| | CcaCas13 | | | ID. NO: | NO: 2909) | | |
| | b 7 | | | 2908) | | | |
| 2b | APML | CcaCas13b | gcctccccggcgccactggccacgt | gcctccccg | GTTGGAACTGCT | APML | 2b |
| | long | | ggttgGTTGGAACTGCTCTCATTTT | gcgccactg | CTCATTTTGGAGG | long | |
| | valida- | | GGAGGGTAATCACAAC | gccacgtgg | GTAATCACAAC | | |
| | tion | | (SEQ ID. NO: 2910) | ttg (SEQ | (SEQ ID. | | |
| | CcaCas13 | | | ID. NO: | NO: 2912) | | |
| | b 8 | | | 2911) | | | |
| 2b | APML | CcaCas13b | tgcctccccggcgccactggccacg | tgcctccccg | GTTGGAACTGCT | APML | 2b |
| | long | | tggttGTTGGAACTGCTCTCATTTT | gcgccactg | CTCATTTTGGAGG | long | |
| | valida- | | GGAGGGTAATCACAAC | gccacgtgg | GTAATCACAAC | | |
| | tion | | (SEQ ID. NO: 2913) | tt (SEQ | (SEQ ID. | | |
| | CcaCas13 | | | ID. NO: | NO: 2915) | | |
| | b 9 | | | 2914) | | | |
| 2b | APML | CcaCas13b | ctgcctccccggcgccactggccac | ctgcctcccc | GTTGGAACTGCT | APML | 2b |
| | long | | gtggtGTTGGAACTGCTCTCATTTT | ggcgccact | CTCATTTTGGAGG | long | |
| | valida- | | GGAGGGTAATCACAAC | ggccacgtg | GTAATCACAAC | | |
| | tion | | (SEQ ID. NO: 2916) | gt(SEQ | (SEQ ID. | | |
| | CcaCas13 | | | ID. NO: | NO: 2918) | | |
| | b 10 | | | 2917) | | | |
| 2b | APML | CcaCas13b | gctgcctccccggcgccactggcca | gctgcctccc | GTTGGAACTGCT | APML | 2b |
| | long | | cgtggGTTGGAACTGCTCTCATTTT | cggcgccac | CTCATTTTGGAGG | long | |
| | valida- | | GGAGGGTAATCACAAC | tggccacgt | GTAATCACAAC | | |
| | tion | | (SEQ ID. NO: 2919) | gg (SEQ | (SEQ ID. | | |
| | CcaCas13 | | | ID. NO: | NO: 2921) | | |
| | b 11 | | | 2920) | | | |
| 2b | APML | CcaCas13b | ggctgcctccccggcgccactggcc | ggctgcctc | GTTGGAACTGCT | APML | 2b |
| | long | | acgtgGTTGGAACTGCTCTCATTTT | cccggcgcc | CTCATTTTGGAGG | long | |
| | valida- | | GGAGGGTAATCACAAC | actggccac | GTAATCACAAC | | |
| | tion | | (SEQ ID. NO: 2922) | gtg (SEQ | (SEQ ID. | | |
| | CcaCas13 | | | ID. NO: | NO: 2924) | | |
| | b 12 | | | 2923) | | | |
| 2b | APML | CcaCas13b | tggctgcctccccggcgccactggc | tggctgcctc | GTTGGAACTGCT | APML | 2b |
| | long | | cacgtGTTGGAACTGCTCTCATTTT | cccggcgcc | CTCATTTTGGAGG | long | |
| | valida- | | GGAGGGTAATCACAAC | actggccac | GTAATCACAAC | | |
| | tion | | (SEQ ID. NO: 2925) | gt (SEQ | (SEQ ID. | | |
| | CcaCas13 | | | ID. NO: | NO: 2927) | | |
| | b 13 | | | 2926) | | | |
| 2b | APML | CcaCas13b | atggctgcctccccggcgccactgg | atggctgcct | GTTGGAACTGCT | APML | 2b |
| | long | | ccacgGTTGGAACTGCTCTCATTTT | ccccggcgc | CTCATTTTGGAGG | long | |
| | valida- | | GGAGGGTAATCACAAC | cactggcca | GTAATCACAAC | | |
| | tion | | (SEQ ID. NO: 2928) | cg (SEQ | (SEQ ID. | | |
| | CcaCas13 | | | ID. NO: | NO: 2930) | | |
| | b 14 | | | 2929) | | | |
| 2b | APML | CcaCas13b | aatggctgcctccccggcgccactg | aatggctgc | GTTGGAACTGCT | APML | 2b |
| | long | | gccacGTTGGAACTGCTCTCATTTT | ctccccggc | CTCATTTTGGAGG | long | |
| | valida- | | GGAGGGTAATCACAAC | gccactggc | GTAATCACAAC | | |
| | tion | | (SEQ ID. NO: 2931) | cac (SEQ | (SEQ ID. | | |
| | CcaCas13 | | | ID. NO: | NO: 2933) | | |
| | b 15 | | | 2932) | | | |
| 2b | APML | CcaCas13b | caatggctgcctccccggcgccact | caatggctg | GTTGGAACTGCT | APML | 2b |
| | long | | ggccaGTTGGAACTGCTCTCATTTT | cctccccgg | CTCATTTTGGAGG | long | |
| | valida- | | GGAGGGTAATCACAAC | cgccactgg | GTAATCACAAC | | |
| | tion | | (SEQ ID. NO: 2934) | cca (SEQ | (SEQ ID. | | |
| | CcaCas13 | | | ID. NO: | NO: 2936) | | |
| | b 16 | | | 2935) | | | |
| 2b | APML | CcaCas13b | ctgggtgatgcaagagctgaggtcc | ctgggtgatg | GTTGGAACTGCT | APML | 2b |
| | short | | tgcagGTTGGAACTGCTCTCATTTT | caagagctg | CTCATTTTGGAGG | short | |
| | valida- | | GGAGGGTAATCACAAC | aggtcctgc | GTAATCACAAC | | |
| | tion | | (SEQ ID. NO: 2937) | ag (SEQ | (SEQ ID. | | |
| | CcaCas13 | | | ID. NO: | NO: 2939) | | |
| | b 1 | | | 2938) | | | |
| 2b | APML | CcaCas13b | cctgggtgatgcaagagctgaggtc | cctgggtgat | GTTGGAACTGCT | APML | 2b |
| | short | | ctgcaGTTGGAACTGCTCTCATTTT | gcaagagct | CTCATTTTGGAGG | short | |
| | valida- | | GGAGGGTAATCACAAC | gaggtcctg | GTAATCACAAC | | |
| | tion | | (SEQ ID. NO: 2940) | ca (SEQ | (SEQ ID. | | |
| | CcaCas13 | | | ID. NO: | NO: 2942) | | |
| | b 2 | | | 2941) | | | |
| 2b | APML | CcaCas13b | ccctgggtgatgcaagagctgaggt | ccctgggtg | GTTGGAACTGCT | APML | 2b |
| | short | | cctgcGTTGGAACTGCTCTCATTTT | atgcaagag | CTCATTTTGGAGG | short | |
| | valida- | | GGAGGGTAATCACAAC | ctgaggtcct | GTAATCACAAC | | |
| | tion | | (SEQ ID. NO: 2943) | gc (SEQ | (SEQ ID. | | |
| | CcaCas13 | | | ID. NO: | NO: 2945) | | |
| | b 3 | | | 2944) | | | |
| 2b | APML | CcaCas13b | cccctgggtgatgcaagagctgagg | cccctgggt | GTTGGAACTGCT | APML | 2b |
| | short | | tcctgGTTGGAACTGCTCTCATTTT | gatgcaaga | CTCATTTTGGAGG | short | |
| | valida- | | GGAGGGTAATCACAAC | gctgaggtc | GTAATCACAAC | | |
| | tion | | (SEQ ID. NO: 2946) | ctg (SEQ | (SEQ ID. | | |
| | CcaCas13 | | | ID. NO: | NO: 2948) | | |
| | b 4 | | | 2947) | | | |
| 2b | APML | CcaCas13b | tcccctgggtgatgcaagagctgag | tcccctgggt | GTTGGAACTGCT | APML | 2b |
| | short | | gtcctGTTGGAACTGCTCTCATTTT | gatgcaaga | CTCATTTTGGAGG | short | |
| | valida- | | GGAGGGTAATCACAAC | gctgaggtc | GTAATCACAAC | | |
| | tion | | (SEQ ID. NO: 2949) | ct (SEQ | (SEQ ID. | | |
| | CcaCas13 | | | ID. NO: | NO: 2951) | | |
| | b 5 | | | 2950) | | | |
| 2b | APML | CcaCas13b | ttcccctgggtgatgcaagagctga | ttcccctggg | GTTGGAACTGCT | APML | 2b |
| | short | | ggtccGTTGGAACTGCTCTCATTTT | tgatgcaag | CTCATTTTGGAGG | short | |
| | valida- | | GGAGGGTAATCACAAC | agctgaggt | GTAATCACAAC | | |
| | tion | | (SEQ ID. NO: 2952) | cc (SEQ | (SEQ ID. | | |
| | CcaCas13 | | | ID. NO: | NO: 2954) | | |
| | b 6 | | | 2953) | | | |
| 2b | APML | CcaCas13b | tttcccctgggtgatgcaagagctg | tttcccctgg | GTTGGAACTGCT | APML | 2b |
| | short | | aggtcGTTGGAACTGCTCTCATTTT | gtgatgcaa | CTCATTTTGGAGG | short | |
| | valida- | | GGAGGGTAATCACAAC | gagctgagg | GTAATCACAAC | | |
| | tion | | (SEQ ID. NO: 2955) | tc (SEQ | (SEQ ID. | | |
| | CcaCas13 | | | ID. NO: | NO: 2957) | | |
| | b 7 | | | 2956) | | | |
| 2b | APML | CcaCas13b | ctttcccctgggtgatgcaagagct | ctttcccctg | GTTGGAACTGCT | APML | 2b |
| | short | | gaggtGTTGGAACTGCTCTCATTTT | ggtgatgca | CTCATTTTGGAGG | short | |
| | valida- | | GGAGGGTAATCACAAC | agagctgag | GTAATCACAAC | | |
| | tion | | (SEQ ID. NO: 2958) | gt (SEQ | (SEQ ID. | | |
| | CcaCas13 | | | ID. NO: | NO: 2960) | | |
| | b 8 | | | 2959) | | | |
| 2b | APML | CcaCas13b | gctttcccctgggtgatgcaagagc | gctttcccct | GTTGGAACTGCT | APML | 2b |
| | short | | tgaggGTTGGAACTGCTCTCATTTT | gggtgatgc | CTCATTTTGGAGG | short | |
| | valida- | | GGAGGGTAATCACAAC | aagagctga | GTAATCACAAC | | |
| | tion | | (SEQ ID. NO: 2961) | gg (SEQ | (SEQ ID. | | |
| | CcaCas13 | | | ID. NO: | NO: 2963) | | |
| | b 9 | | | 2962) | | | |
| 2b | APML | CcaCas13b | ggctttcccctgggtgatgcaagag | ggctttcccc | GTTGGAACTGCT | APML | 2b |
| | short | | ctgagGTTGGAACTGCTCTCATTTT | tgggtgatgc | CTCATTTTGGAGG | short | |
| | valida- | | GGAGGGTAATCACAAC | aagagctga | GTAATCACAAC | | |
| | tion | | (SEQ ID. NO: 2964) | g (SEQ | (SEQ ID. | | |
| | CcaCas13 | | | ID. NO: | NO: 2966) | | |
| | b 10 | | | 2965) | | | |
| 2b | APML | CcaCas13b | tggctttcccctgggtgatgcaaga | tggctttccc | GTTGGAACTGCT | APML | 2b |
| | short | | gctgaGTTGGAACTGCTCTCATTTT | ctgggtgatg | CTCATTTTGGAGG | short | |
| | valida- | | GGAGGGTAATCACAAC | caagagctg | GTAATCACAAC | | |
| | tion | | (SEQ ID. NO: 2967) | a (SEQ | (SEQ ID. | | |
| | CcaCas13 | | | ID. NO: | NO: 2969) | | |
| | b 11 | | | 2968) | | | |
| 2b | APML | CcaCas13b | atggctttcccctgggtgatgcaag | atggctttcc | GTTGGAACTGCT | APML | 2b |
| | short | | agctgGTTGGAACTGCTCTCATTTT | cctgggtgat | CTCATTTTGGAGG | short | |
| | valida- | | GGAGGGTAATCACAAC | gcaagagct | GTAATCACAAC | | |
| | tion | | (SEQ ID. NO: 2970) | g (SEQ | (SEQ ID. | | |
| | CcaCas13 | | | ID. NO: | NO: 2972) | | |
| | b 12 | | | 2971) | | | |
| 2b | APML | CcaCas13b | aatggctttcccctgggtgatgcaa | aatggctttc | GTTGGAACTGCT | APML | 2b |
| | short | | gagctGTTGGAACTGCTCTCATTTT | ccctgggtg | CTCATTTTGGAGG | short | |
| | valida- | | GGAGGGTAATCACAAC | atgcaagag | GTAATCACAAC | | |
| | tion | | (SEQ ID. NO: 2973) | ct (SEQ | (SEQ ID. | | |
| | CcaCas13 | | | ID. NO: | NO: 2975) | | |
| | b 13 | | | 2974) | | | |
| 2b | APML | CcaCas13b | caatggctttcccctgggtgatgca | caatggcttt | GTTGGAACTGCT | APML | 2b |
| | short | | agagcGTTGGAACTGCTCTCATTTT | cccctgggt | CTCATTTTGGAGG | short | |
| | valida- | | GGAGGGTAATCACAAC | gatgcaaga | GTAATCACAAC | | |
| | tion | | (SEQ ID. NO: 2976) | gc (SEQ | (SEQ ID. | | |
| | CcaCas13 | | | ID. NO: | NO: 2978) | | |
| | b 14 | | | 2977) | | | |
| 2b | APML | CcaCas13b | tcaatggctttcccctgggtgatgc | tcaatggcttt | GTTGGAACTGCT | APML | 2b |
| | short | | aagagGTTGGAACTGCTCTCATTTT | cccctgggt | CTCATTTTGGAGG | short | |
| | valida- | | GGAGGGTAATCACAAC | gatgcaaga | GTAATCACAAC | | |
| | tion | | (SEQ ID. NO: 2979) | g (SEQ | (SEQ ID. | | |
| | CcaCas13 | | | ID. NO: | NO: 2981) | | |
| | b 15 | | | 2980) | | | |
| 2b | APML | CcaCas13b | ctcaatggctttcccctgggtgatg | ctcaatggct | GTTGGAACTGCT | APML | 2b |
| | short | | caagaGTTGGAACTGCTCTCATTTT | ttcccctggg | CTCATTTTGGAGG | short | |
| | valida- | | GGAGGGTAATCACAAC | tgatgcaag | GTAATCACAAC | | |
| | tion | | (SEQ ID. NO: 2982) | a (SEQ | (SEQ ID. | | |
| | CcaCas13 | | | ID. NO: | NO: 2984) | | |
| | b 16 | | | 2983) | | | |
| 2b | APML | CcaCas13b | tctcaatggctttcccctgggtgat | tctcaatggc | GTTGGAACTGCT | APML | 2b |
| | short | | gcaagGTTGGAACTGCTCTCATTTT | tttcccctgg | CTCATTTTGGAGG | short | |
| | valida- | | GGAGGGTAATCACAAC | gtgatgcaa | GTAATCACAAC | | |
| | tion | | (SEQ ID. NO: 2985) | g (SEQ | (SEQ ID. | | |
| | CcaCas13 | | | ID. NO: | NO: 2987) | | |
| | b 17 | | | 2986) | | | |
| 2b | APML | CcaCas13b | gtctcaatggctttcccctgggtga | gtctcaatgg | GTTGGAACTGCT | APML | 2b |
| | short | | tgcaaGTTGGAACTGCTCTCATTTT | ctttcccctg | CTCATTTTGGAGG | short | |
| | valida- | | GGAGGGTAATCACAAC | ggtgatgca | GTAATCACAAC | | |
| | tion | | (SEQ ID. NO: 2988) | a (SEQ | (SEQ ID. | | |
| | CcaCas13 | | | ID. NO: | NO: 2990) | | |
| | b 18 | | | 2989) | | | |
| 2b | APML | CcaCas13b | ggtctcaatggctttcccctgggtg | ggtctcaatg | GTTGGAACTGCT | APML | 2b |
| | short | | atgcaGTTGGAACTGCTCTCATTTT | gctttcccct | CTCATTTTGGAGG | short | |
| | valida- | | GGAGGGTAATCACAAC | gggtgatgc | GTAATCACAAC | | |
| | tion | | (SEQ ID. NO: 2991) | a (SEQ | (SEQ ID. | | |
| | CcaCas13 | | | ID. NO: | NO: 2993) | | |
| | b 19 | | | 2992) | | | |
| 2b | APML | CcaCas13b | gggtctcaatggctttcccctgggt | gggtctcaat | GTTGGAACTGCT | APML | 2b |
| | short | | gatgcGTTGGAACTGCTCTCATTTT | ggctttcccc | CTCATTTTGGAGG | short | |
| | valida- | | GGAGGGTAATCACAAC | tgggtgatgc | GTAATCACAAC | | |
| | tion | | (SEQ ID. NO: 2994) | (SEQ ID. | (SEQ ID. | | |
| | CcaCas13 | | | NO: | NO: 2996) | | |
| | b 20 | | | 2995) | | | |
| 2b | APML | CcaCas13b | tgggtctcaatggctttcccctggg | tgggtctcaa | GTTGGAACTGCT | APML | 2b |
| | short | | tgatgGTTGGAACTGCTCTCATTTT | tggctttccc | CTCATTTTGGAGG | short | |
| | valida- | | GGAGGGTAATCACAAC | ctgggtgatg | GTAATCACAAC | | |
| | tion | | (SEQ ID. NO: 2997) | (SEQ ID. | (SEQ ID. | | |
| | CcaCas13 | | | NO: | NO: 2999) | | |
| | b 21 | | | 2998) | | | |
| 2b | APML | CcaCas13b | ctgggtctcaatggctttcccctgg | ctgggtctca | GTTGGAACTGCT | APML | 2b |
| | short | | gtgatGTTGGAACTGCTCTCATTTT | atggctttcc | CTCATTTTGGAGG | short | |
| | valida- | | GGAGGGTAATCACAAC | cctgggtgat | GTAATCACAAC | | |
| | tion | | (SEQ ID. NO: 3000) | (SEQ ID. | (SEQ ID. | | |
| | CcaCas13 | | | NO: | NO: 3002) | | |
| | b 22 | | | 3001) | | | |
| 2b | Thermo- | CcaCas13b | tcattggttgacctttgtacattaa | tcattggttga | GTTGGAACTGCT | Ther- | 2b |
| | nuclease | | tttaaGTTGGAACTGCTCTCATTTT | cctttgtacat | CTCATTTTGGAGG | mo- | |
| | valida- | | GGAGGGTAATCACAAC | taatttaa | GTAATCACAAC | nu- | |
| | tion | | (SEQ ID. NO: 3003) | (SEQ ID. | (SEQ ID. | clease | |
| | CcaCas13 | | | NO: | NO: 3005) | | |
| | b 1 | | | 3004) | | | |
| 2b | Thermo- | CcaCas13b | tgtcattggttgacctttgtacatt | tgtcattggtt | GTTGGAACTGCT | Ther- | 2b |
| | nuclease | | aatttGTTGGAACTGCTCTCATTTT | gacctttgta | CTCATTTTGGAGG | mo- | |
| | valida- | | GGAGGGTAATCACAAC | cattaattt | GTAATCACAAC | nu- | |
| | tion | | (SEQ ID. NO: 3006) | (SEQ ID. | (SEQ ID. | clease | |
| | CcaCas13 | | | NO: | NO: 3008) | | |
| | b 2 | | | 3007) | | | |
| 2b | Thermo- | CcaCas13b | aatgtcattggttgacctttgtaca | aatgtcattg | GTTGGAACTGCT | Ther- | 2b |
| | nuclease | | ttaatGTTGGAACTGCTCTCATTTT | gttgacctttg | CTCATTTTGGAGG | mo- | |
| | valida- | | GGAGGGTAATCACAAC | tacattaat | GTAATCACAAC | nu- | |
| | tion | | (SEQ ID. NO: 3009) | (SEQ ID. | (SEQ ID. | clease | |
| | CcaCas13 | | | NO: | NO: 3011) | | |
| | b 3 | | | 3010) | | | |
| 2b | Thermo- | CcaCas13b | tgaatgtcattggttgacctttgta | tgaatgtcatt | GTTGGAACTGCT | Ther- | 2b |
| | nuclease | | cattaGTTGGAACTGCTCTCATTTT | ggttgaccttt | CTCATTTTGGAGG | mo- | |
| | valida- | | GGAGGGTAATCACAAC | gtacatta | GTAATCACAAC | nu- | |
| | tion | | (SEQ ID. NO: 3012) | (SEQ ID. | (SEQ ID. | clease | |
| | CcaCas13 | | | NO: | NO: 3014) | | |
| | b 4 | | | 3013) | | | |
| 2b | Thermo- | CcaCas13b | tctgaatgtcattggttgacctttg | tctgaatgtc | GTTGGAACTGCT | Ther- | 2b |
| | nuclease | | tacatGTTGGAACTGCTCTCATTTT | attggttgac | CTCATTTTGGAGG | mo- | |
| | valida- | | GGAGGGTAATCACAAC | ctttgtacat | GTAATCACAAC | nu- | |
| | tion | | (SEQ ID. NO: 3015) | (SEQ ID. | (SEQ ID. | clease | |
| | CcaCas13 | | | NO: | NO: 3017) | | |
| | b 5 | | | 3016) | | | |
| 2b | Thermo- | CcaCas13b | agtctgaatgtcattggttgacctt | agtctgaatg | GTTGGAACTGCT | Ther- | 2b |
| | nuclease | | tgtacGTTGGAACTGCTCTCATTTT | tcattggttga | CTCATTTTGGAGG | mo- | |
| | valida- | | GGAGGGTAATCACAAC | cctttgtac | GTAATCACAAC | nu- | |
| | tion | | (SEQ ID. NO: 3018) | (SEQ ID. | (SEQ ID. | clease | |
| | CcaCas13 | | | NO: | NO: 3020) | | |
| | b 6 | | | 3019) | | | |
| 2b | Thermo- | CcaCas13b | atagtctgaatgtcattggttgacc | atagtctgaa | GTTGGAACTGCT | Ther- | 2b |
| | nuclease | | tttgtGTTGGAACTGCTCTCATTTT | tgtcattggtt | CTCATTTTGGAGG | mo- | |
| | valida- | | GGAGGGTAATCACAAC | gacctttgt | GTAATCACAAC | nu- | |
| | tion | | (SEQ ID. NO: 3021) | (SEQ ID. | (SEQ ID. | clease | |
| | CcaCas13 | | | NO: | NO: 3023) | | |
| | b 7 | | | 3022) | | | |
| 2b | Thermo- | CcaCas13b | taatagtctgaatgtcattggttga | taatagtctg | GTTGGAACTGCT | Ther- | 2b |
| | nuclease | | cctttGTTGGAACTGCTCTCATTTT | aatgtcattg | CTCATTTTGGAGG | mo- | |
| | valida- | | GGAGGGTAATCACAAC | gttgaccttt | GTAATCACAAC | nu- | |
| | tion | | (SEQ ID. NO: 3024) | (SEQ ID. | (SEQ ID. | clease | |
| | CcaCasl3 | | | NO: | NO: 3026) | | |
| | b 8 | | | 3025) | | | |
| 2b | Thermo- | CcaCas13b | aataatagtctgaatgtcattggtt | aataatagtc | GTTGGAACTGCT | Ther- | 2b |
| | nuclease | | gacctGTTGGAACTGCTCTCATTTT | tgaatgtcatt | CTCATTTTGGAGG | mo- | |
| | valida- | | GGAGGGTAATCACAAC | ggttgacct | GTAATCACAAC | nu- | |
| | tion | | (SEQ ID. NO: 3027) | (SEQ ID. | (SEQ ID. | clease | |
| | CcaCas13 | | | NO: | NO: 3029) | | |
| | b 9 | | | 3028) | | | |
| 2b | Thermo- | CcaCas13b | ccaataatagtctgaatgtcattgg | ccaataatag | GTTGGAACTGCT | Ther- | 2b |
| | nuclease | | ttgacGTTGGAACTGCTCTCATTTT | tctgaatgtc | CTCATTTTGGAGG | mo- | |
| | valida- | | GGAGGGTAATCACAAC | attggttgac | GTAATCACAAC | nu- | |
| | tion | | (SEQ ID. NO: 3030) | (SEQ ID. | (SEQ ID. | clease | |
| | CcaCas13 | | | NO: | NO: 3032) | | |
| | b 10 | | | 3031) | | | |
| 2b | Thermo- | CcaCas13b | aaccaataatagtctgaatgtcatt | aaccaataat | GTTGGAACTGCT | Ther- | 2b |
| | nuclease | | ggttgGTTGGAACTGCTCTCATTTT | agtctgaatg | CTCATTTTGGAGG | mo- | |
| | valida- | | GGAGGGTAATCACAAC | tcattggttg | GTAATCACAAC | nu- | |
| | tion | | (SEQ ID. NO: 3033) | (SEQ ID. | (SEQ ID. | clease | |
| | CcaCas13 | | | NO: | NO: 3035) | | |
| | b 11 | | | 3034) | | | |
| 2b | Thermo- | CcaCas13b | tcaaccaataatagtctgaatgtca | tcaaccaata | GTTGGAACTGCT | Ther- | 2b |
| | nuclease | | ttggtGTTGGAACTGCTCTCATTTT | atagtctgaa | CTCATTTTGGAGG | mo- | |
| | valida- | | GGAGGGTAATCACAAC | tgtcattggt | GTAATCACAAC | nu- | |
| | tion | | (SEQ ID. NO: 3036) | (SEQ ID. | (SEQ ID. | clease | |
| | CcaCas13 | | | NO: | NO: 3038) | | |
| | b 12 | | | 3037) | | | |
| 2b | Thermo- | CcaCas13b | tatcaaccaataatagtctgaatgt | tatcaaccaa | GTTGGAACTGCT | Ther- | 2b |
| | nuclease | | cattgGTTGGAACTGCTCTCATTTT | taatagtctg | CTCATTTTGGAGG | mo- | |
| | valida- | | GGAGGGTAATCACAAC | aatgtcattg | GTAATCACAAC | nu- | |
| | tion | | (SEQ ID. NO: 3039) | (SEQ ID. | (SEQ ID. | clease | |
| | CcaCas13 | | | NO: | NO: 3041) | | |
| | b 13 | | | 3040) | | | |
| 2b | Thermo- | CcaCas13b | tgtatcaaccaataatagtctgaat | tgtatcaacc | GTTGGAACTGCT | Ther- | 2b |
| | nuclease | | gtcatGTTGGAACTGCTCTCATTTT | aataatagtc | CTCATTTTGGAGG | mo- | |
| | valida- | | GGAGGGTAATCACAAC | tgaatgtcat | GTAATCACAAC | nu- | |
| | tion | | (SEQ ID. NO: 3042) | (SEQ ID. | (SEQ ID. | clease | |
| | CcaCas13 | | | NO: | NO: 3044) | | |
| | b 14 | | | 3043) | | | |
| 2b | Thermo- | CcaCas13b | ggtgtatcaaccaataatagtctga | ggtgtatcaa | GTTGGAACTGCT | Ther- | 2b |
| | nuclease | | atgtcGTTGGAACTGCTCTCATTTT | ccaataatag | CTCATTTTGGAGG | mo- | |
| | valida- | | GGAGGGTAATCACAAC | tctgaatgtc | GTAATCACAAC | nu- | |
| | tion | | (SEQ ID. NO: 3045) | (SEQ ID. | (SEQ ID. | clease | |
| | CcaCas13 | | | NO: | NO: 3047) | | |
| | b 15 | | | 3046) | | | |
| 2b | Thermo- | CcaCas13b | caggtgtatcaaccaataatagtct | caggtgtatc | GTTGGAACTGCT | Ther- | 2b |
| | nuclease | | gaatgGTTGGAACTGCTCTCATTTT | aaccaataat | CTCATTTTGGAGG | mo- | |
| | valida- | | GGAGGGTAATCACAAC | agtctgaatg | GTAATCACAAC | nu- | |
| | tion | | (SEQ ID. NO: 3048) | (SEQ ID. | (SEQ ID. | clease | |
| | CcaCas13 | | | NO: | NO: 3050) | | |
| | b 16 | | | 3049) | | | |
| 2b | Thermo- | CcaCas13b | ttcaggtgtatcaaccaataatagt | ttcaggtgtat | GTTGGAACTGCT | Ther- | 2b |
| | nuclease | | ctgaaGTTGGAACTGCTCTCATTTT | caaccaata | CTCATTTTGGAGG | mo- | |
| | valida- | | GGAGGGTAATCACAAC | atagtctgaa | GTAATCACAAC | nu- | |
| | tion | | (SEQ ID. NO: 3051) | (SEQ ID. | (SEQ ID. | clease | |
| | CcaCas13 | | | NO: | NO: 3053) | | |
| | b 17 | | | 3052) | | | |
| 2b | Thermo- | CcaCas13b | gtttcaggtgtatcaaccaataata | gtttcaggtg | GTTGGAACTGCT | Ther- | 2b |
| | nuclease | | gtctgGTTGGAACTGCTCTCATTTT | tatcaaccaa | CTCATTTTGGAGG | mo- | |
| | valida- | | GGAGGGTAATCACAAC | taatagtctg | GTAATCACAAC | nu- | |
| | tion | | (SEQ ID. NO: 3054) | (SEQ ID. | (SEQ ID. | clease | |
| | CcaCas13 | | | NO: | NO: 3056) | | |
| | b 18 | | | 3055) | | | |
| 2b | Thermo- | CcaCas13b | ttgtttcaggtgtatcaaccaataa | ttgtttcaggt | GTTGGAACTGCT | Ther- | 2b |
| | nuclease | | tagtcGTTGGAACTGCTCTCATTTT | gtatcaacca | CTCATTTTGGAGG | mo- | |
| | valida- | | GGAGGGTAATCACAAC | ataatagtc | GTAATCACAAC | nu- | |
| | tion | | (SEQ ID. NO: 3057) | (SEQ ID. | (SEQ ID. | clease | |
| | CcaCas13 | | | NO: | NO: 3059) | | |
| | b 19 | | | 3058) | | | |
| 2b | Thermo- | CcaCas13b | ctttgtttcaggtgtatcaaccaat | ctttgtttcag | GTTGGAACTGCT | Ther- | 2b |
| | nuclease | | aatagGTTGGAACTGCTCTCATTTT | gtgtatcaac | CTCATTTTGGAGG | mo- | |
| | valida- | | GGAGGGTAATCACAAC | caataatag | GTAATCACAAC | nu- | |
| | tion | | (SEQ ID. NO: 3060) | (SEQ ID. | (SEQ ID. | clease | |
| | CcaCas13 | | | NO: | NO: 3062) | | |
| | b 20 | | | 3061) | | | |
| 2b | Thermo- | CcaCas13b | tgctttgtttcaggtgtatcaacca | tgctttgtttc | GTTGGAACTGCT | Ther- | 2b |
| | nuclease | | ataatGTTGGAACTGCTCTCATTTT | aggtgtatca | CTCATTTTGGAGG | mo- | |
| | valida- | | GGAGGGTAATCACAAC | accaataat | GTAATCACAAC | nu- | |
| | tion | | (SEQ ID. NO: 3063) | (SEQ ID. | (SEQ ID. | clease | |
| | CcaCas13 | | | NO: | NO: 3065) | | |
| | b 21 | | | 3064) | | | |
| 2b | Thermo- | CcaCas13b | gatgctttgtttcaggtgtatcaac | gatgctttgtt | GTTGGAACTGCT | Ther- | 2b |
| | nuclease | | caataGTTGGAACTGCTCTCATTTT | tcaggtgtat | CTCATTTTGGAGG | mo- | |
| | valida- | | GGAGGGTAATCACAAC | caaccaata | GTAATCACAAC | nu- | |
| | tion | | (SEQ ID. NO: 3066) | (SEQ ID. | (SEQ ID. | clease | |
| | CcaCas13 | | | NO: | NO: 3068) | | |
| | b 22 | | | 3067) | | | |
| 2b | Thermo- | CcaCas13b | aggatgctttgtttcaggtgtatca | aggatgcttt | GTTGGAACTGCT | Ther- | 2b |
| | nuclease | | accaaGTTGGAACTGCTCTCATTTT | gtttcaggtg | CTCATTTTGGAGG | mo- | |
| | valida- | | GGAGGGTAATCACAAC | tatcaaccaa | GTAATCACAAC | nu- | |
| | tion | | (SEQ ID. NO: 3069) | (SEQ ID. | (SEQ ID. | clease | |
| | CcaCas13 | | | NO: | NO: 3071) | | |
| | b 23 | | | 3070) | | | |
| 2b | Thermo- | CcaCas13b | ttaggatgctttgtttcaggtgtat | ttaggatgctt | GTTGGAACTGCT | Ther- | 2b |
| | nuclease | | caaccGTTGGAACTGCTCTCATTTT | tgtttcaggt | CTCATTTTGGAGG | mo- | |
| | valida- | | GGAGGGTAATCACAAC | gtatcaacc | GTAATCACAAC | nu- | |
| | tion | | (SEQ ID. NO: 3072) | (SEQ ID. | (SEQ ID. | clease | |
| | CcaCas13 | | | NO: | NO: 3074) | | |
| | b 24 | | | 3073) | | | |
| 2b | Thermo- | CcaCas13b | ttttaggatgctttgtttcaggtgt | ttttaggatgc | GTTGGAACTGCT | Ther- | 2b |
| | nuclease | | atcaaGTTGGAACTGCTCTCATTTT | tttgtttcagg | CTCATTTTGGAGG | mo- | |
| | valida- | | GGAGGGTAATCACAAC | tgtatcaa | GTAATCACAAC | nu- | |
| | tion | | (SEQ ID. NO: 3075) | (SEQ ID. | (SEQ ID. | clease | |
| | CcaCas13 | | | NO: | NO: 3077) | | |
| | b 25 | | | 3076) | | | |
| 2b | Thermo- | CcaCas13b | ttttttaggatgctttgtttcaggt | ttttttaggat | GTTGGAACTGCT | Ther- | 2b |
| | nuclease | | gtatcGTTGGAACTGCTCTCATTTT | gctttgtttca | CTCATTTTGGAGG | mo- | |
| | valida- | | GGAGGGTAATCACAAC | ggtgtatc | GTAATCACAAC | nu- | |
| | tion | | (SEQ ID. NO: 3078) | (SEQ ID. | (SEQ ID. | clease | |
| | CcaCas13 | | | NO: | NO: 3080) | | |
| | b 26 | | | 3079) | | | |
| 2b | Thermo- | CcaCas13b | ccttttttaggatgctttgtttcag | ccttttttagg | GTTGGAACTGCT | Ther- | 2b |
| | nuclease | | gtgtaGTTGGAACTGCTCTCATTTT | atgctttgttt | CTCATTTTGGAGG | mo- | |
| | valida- | | GGAGGGTAATCACAAC | caggtgta | GTAATCACAAC | nu- | |
| | tion | | (SEQ ID. NO: 3081) | (SEQ ID. | (SEQ ID. | clease | |
| | CcaCas13 | | | NO: | NO: 3083) | | |
| | b 27 | | | 3082) | | | |
| 2b | Thermo- | CcaCas13b | caccttttttaggatgctttgtttc | cacctttttta | GTTGGAACTGCT | Ther- | 2b |
| | nuclease | | aggtgGTTGGAACTGCTCTCATTTT | ggatgctttg | CTCATTTTGGAGG | mo- | |
| | valida- | | GGAGGGTAATCACAAC | tttcaggtg | GTAATCACAAC | nu- | |
| | tion | | (SEQ ID. NO: 3084) | (SEQ ID. | (SEQ ID. | clease | |
| | CcaCas13 | | | NO: | NO: 3086) | | |
| | b 28 | | | 3085) | | | |
| 2b | Thermo- | CcaCas13b | tacaccttttttaggatgctttgtt | tacaccttttt | GTTGGAACTGCT | Ther- | 2b |
| | nuclease | | tcaggGTTGGAACTGCTCTCATTTT | taggatgcttt | CTCATTTTGGAGG | mo- | |
| | valida- | | GGAGGGTAATCACAAC | gtticagg | GTAATCACAAC | nu- | |
| | tion | | (SEQ ID. NO: 3087) | (SEQ ID. | (SEQ ID. | clease | |
| | CcaCas13 | | | NO: | NO: 3089) | | |
| | b 29 | | | 3088) | | | |
| 2b | Thermo- | CcaCas13b | tctacaccttttttaggatgctttg | tctacaccttt | GTTGGAACTGCT | Ther- | 2b |
| | nuclease | | tttcaGTTGGAACTGCTCTCATTTT | tttaggatgct | CTCATTTTGGAGG | mo- | |
| | valida- | | GGAGGGTAATCACAAC | ttgtttca | GTAATCACAAC | nu- | |
| | tion | | (SEQ ID. NO: 3090) | (SEQ ID. | (SEQ ID. | clease | |
| | CcaCas13 | | | NO: | NO: 3092) | | |
| | b 30 | | | 3091) | | | |
| 2b | Thermo- | CcaCas13b | tctctacaccttttttaggatgctt | tctctacacct | GTTGGAACTGCT | Ther- | 2b |
| | nuclease | | tgtttGTTGGAACTGCTCTCATTTT | tttttaggatg | CTCATTTTGGAGG | mo- | |
| | valida- | | GGAGGGTAATCACAAC | ctttgttt | GTAATCACAAC | nu- | |
| | tion | | (SEQ ID. NO: 3093) | (SEQ ID. | (SEQ ID. | clease | |
| | CcaCas13 | | | NO: | NO: 3095) | | |
| | b 31 | | | 3094) | | | |
| 2b | Thermo- | CcaCas13b | tttctctacaccttttttaggatgc | tttctctacac | GTTGGAACTGCT | Ther- | 2b |
| | nuclease | | tttgtGTTGGAACTGCTCTCATTTT | cttttttagga | CTCATTTTGGAGG | mo- | |
| | valida- | | GGAGGGTAATCACAAC | tgctttgt | GTAATCACAAC | nu- | |
| | tion | | (SEQ ID. NO: 3096) | (SEQ ID. | (SEQ ID. | clease | |
| | CcaCas13 | | | NO: | NO: 3098) | | |
| | b 32 | | | 3097) | | | |
| 2b | Thermo- | CcaCas13b | tatttctctacaccttttttaggat | tatttctctac | GTTGGAACTGCT | Ther- | 2b |
| | nuclease | | gctttGTTGGAACTGCTCTCATTTT | accttttttag | CTCATTTTGGAGG | mo- | |
| | valida- | | GGAGGGTAATCACAAC | gatgcttt | GTAATCACAAC | nu- | |
| | tion | | (SEQ ID. NO: 3099) | (SEQ ID. | (SEQ ID. | clease | |
| | CcaCas13 | | | NO: | NO: 3101) | | |
| | b 33 | | | 3100) | | | |
| 2b | Thermo- | CcaCas13b | catatttctctacaccttttttagg | catatttctct | GTTGGAACTGCT | Ther- | 2b |
| | nuclease | | atgctGTTGGAACTGCTCTCATTTT | acacctttttt | CTCATTTTGGAGG | mo- | |
| | valida- | | GGAGGGTAATCACAAC | aggatgct | GTAATCACAAC | nu- | |
| | tion | | (SEQ ID. NO: 3102) | (SEQ ID. | (SEQ ID. | clease | |
| | CcaCas13 | | | NO: | NO: 3104) | | |
| | b 34 | | | 3103) | | | |
| 2b | Thermo- | CcaCas13b | accatatttctctacacctttttta | accatatttct | GTTGGAACTGCT | Ther- | 2b |
| | nuclease | | ggatgGTTGGAACTGCTCTCATTTT | ctacacctttt | CTCATTTTGGAGG | mo- | |
| | valida- | | GGAGGGTAATCACAAC | ttaggatg | GTAATCACAAC | nu- | |
| | tion | | (SEQ ID. NO: 3105) | (SEQ ID. | (SEQ ID. | clease | |
| | CcaCas13 | | | NO: | NO: 3107) | | |
| | b 35 | | | 3106) | | | |
| 2b | Thermo- | CcaCas13b | ggaccatatttctctacaccttttt | ggaccatatt | GTTGGAACTGCT | Ther- | 2b |
| | nuclease | | taggaGTTGGAACTGCTCTCATTTT | tctctacacct | CTCATTTTGGAGG | mo- | |
| | valida- | | GGAGGGTAATCACAAC | tttttagga | GTAATCACAAC | nu- | |
| | tion | | (SEQ ID. NO: 3108) | (SEQ ID. | (SEQ ID. | clease | |
| | CcaCas13 | | | NO: | NO: 3110) | | |
| | b 36 | | | 3109) | | | |
| 2b | Thermo- | CcaCas13b | caggaccatatttctctacaccttt | caggaccat | GTTGGAACTGCT | Ther- | 2b |
| | nuclease | | tttagGTTGGAACTGCTCTCATTTT | atttctctaca | CTCATTTTGGAGG | mo- | |
| | valida- | | GGAGGGTAATCACAAC | ccttttttag | GTAATCACAAC | nu- | |
| | tion | | (SEQ ID. NO: 3111) | (SEQ ID. | (SEQ ID. | clease | |
| | CcaCas13 | | | NO: | NO: 3113) | | |
| | b 37 | | | 3112) | | | |
| 2b | Thermo- | CcaCas13b | ttcaggaccatatttctctacacct | ttcaggacca | GTTGGAACTGCT | Ther- | 2b |
| | nuclease | | tttttGTTGGAACTGCTCTCATTTT | tatttctctac | CTCATTTTGGAGG | mo- | |
| | valida- | | GGAGGGTAATCACAAC | acctttttt | GTAATCACAAC | nu- | |
| | tion | | (SEQ ID. NO: 3114) | (SEQ ID. | (SEQ ID. | clease | |
| | CcaCas13 | | | NO: | NO: 3116) | | |
| | b 38 | | | 3115) | | | |
| 2b | Thermo- | CcaCas13b | gcttcaggaccatatttctctacac | gcttcagga | GTTGGAACTGCT | Ther- | 2b |
| | nuclease | | cttttGTTGGAACTGCTCTCATTTT | ccatatttctc | CTCATTTTGGAGG | mo- | |
| | valida- | | GGAGGGTAATCACAAC | tacacctttt | GTAATCACAAC | nu- | |
| | tion | | (SEQ ID. NO: 3117) | (SEQ ID. | (SEQ ID. | clease | |
| | CcaCas13 | | | NO: | NO: 3119) | | |
| | b 39 | | | 3118) | | | |
| 2b | Thermo- | CcaCas13b | ttgcttcaggaccatatttctctac | ttgcttcagg | GTTGGAACTGCT | Ther- | 2b |
| | nuclease | | accttGTTGGAACTGCTCTCATTTT | accatatttct | CTCATTTTGGAGG | mo- | |
| | valida- | | GGAGGGTAATCACAAC | ctacacctt | GTAATCACAAC | nu- | |
| | tion | | (SEQ ID. NO: 3120) | (SEQ ID. | (SEQ ID. | clease | |
| | CcaCas13 | | | NO: | NO: 3122) | | |
| | b 40 | | | 3121) | | | |
| 2b | Thermo- | CcaCas13b | acttgcttcaggaccatatttctct | acttgcttca | GTTGGAACTGCT | Ther- | 2b |
| | nuclease | | acaccGTTGGAACTGCTCTCATTTT | ggaccatatt | CTCATTTTGGAGG | mo- | |
| | valida- | | GGAGGGTAATCACAAC | tctctacacc | GTAATCACAAC | nu- | |
| | tion | | (SEQ ID. NO: 3123) | (SEQ ID. | (SEQ ID. | clease | |
| | CcaCas13 | | | NO: | NO: 3125) | | |
| | b 41 | | | 3124) | | | |
| 2b | Thermo- | CcaCas13b | gcacttgcttcaggaccatatttct | gcacttgctt | GTTGGAACTGCT | Ther- | 2b |
| | nuclease | | ctacaGTTGGAACTGCTCTCATTTT | caggaccat | CTCATTTTGGAGG | mo- | |
| | valida- | | GGAGGGTAATCACAAC | atttctctaca | GTAATCACAAC | nu- | |
| | tion | | (SEQ ID. NO: 3126) | (SEQ ID. | (SEQ ID. | clease | |
| | CcaCas13 | | | NO: | NO: 3128) | | |
| | b 42 | | | 3127) | | | |
| 2b | Thermo- | CcaCas13b | atgcacttgcttcaggaccatattt | atgcacttgc | GTTGGAACTGCT | Ther- | 2b |
| | nuclease | | ctctaGTTGGAACTGCTCTCATTTT | ticaggacca | CTCATTTTGGAGG | mo- | |
| | valida- | | GGAGGGTAATCACAAC | tatttctcta | GTAATCACAAC | nu- | |
| | tion | | (SEQ ID. NO: 3129) | (SEQ ID. | (SEQ ID. | clease | |
| | CcaCas13 | | | NO: | NO: 3131) | | |
| | b 43 | | | 3130) | | | |
| 2b | Thermo- | CcaCas13b | aaatgcacttgcttcaggaccatat | aaatgcactt | GTTGGAACTGCT | Ther- | 2b |
| | nuclease | | ttctcGTTGGAACTGCTCTCATTTT | gcttcagga | CTCATTTTGGAGG | mo- | |
| | valida- | | GGAGGGTAATCACAAC | ccatatttctc | GTAATCACAAC | nu- | |
| | tion | | (SEQ ID. NO: 3132) | (SEQ ID. | (SEQ ID. | clease | |
| | CcaCas13 | | | NO: | NO: 3134) | | |
| | b 44 | | | 3133) | | | |
| 2b | Thermo- | CcaCas13b | gtaaatgcacttgcttcaggaccat | gtaaatgcac | GTTGGAACTGCT | Ther- | 2b |
| | nuclease | | atttcGTTGGAACTGCTCTCATTTT | ttgcttcagg | CTCATTTTGGAGG | mo- | |
| | valida- | | GGAGGGTAATCACAAC | accatatttc | GTAATCACAAC | nu- | |
| | tion | | (SEQ ID. NO: 3135) | (SEQ ID. | (SEQ ID. | clease | |
| | CcaCas13 | | | NO: | NO: 3137) | | |
| | b 45 | | | 3136) | | | |
| 2b | Thermo- | CcaCas13b | tcaattttctttgcattttctacca | tcaattttctt | GTTGGAACTGCT | Ther- | 2b |
| | nuclease | | tctttGTTGGAACTGCTCTCATTTT | tgcattttcta | CTCATTTTGGAGG | mo- | |
| | valida- | | GGAGGGTAATCACAAC | ccatcttt | GTAATCACAAC | nu- | |
| | tion | | (SEQ ID. NO: 3138) | (SEQ ID. | (SEQ ID. | clease | |
| | CcaCas13 | | | NO: | NO: 3140) | | |
| | b 46 | | | 3139) | | | |
| 2b | Thermo- | CcaCas13b | ttcaattttctttgcattttctacc | ttcaattttct | GTTGGAACTGCT | Ther- | 2b |
| | nuclease | | atcttGTTGGAACTGCTCTCATTTT | ttgcattttct | CTCATTTTGGAGG | mo- | |
| | valida- | | GGAGGGTAATCACAAC | accatctt | GTAATCACAAC | nu- | |
| | tion | | (SEQ ID. NO: 3141) | (SEQ ID. | (SEQ ID. | clease | |
| | CcaCas13 | | | NO: | NO: 3143) | | |
| | b 47 | | | 3142) | | | |
| 2b | Thermo- | CcaCas13b | cttcaattttctttgcattttctac | cttcaattttc | GTTGGAACTGCT | Ther- | 2b |
| | nuclease | | catctGTTGGAACTGCTCTCATTTT | tttgcattttc | CTCATTTTGGAGG | mo- | |
| | valida- | | GGAGGGTAATCACAAC | taccatct | GTAATCACAAC | nu- | |
| | tion | | (SEQ ID. NO: 3144) | (SEQ ID. | (SEQ ID. | clease | |
| | CcaCas13 | | | NO: | NO: 3146) | | |
| | b 48 | | | 3145) | | | |
| 2c | thermo- | LwaCas13a | GATTTAGACTACCCCAAAA | TATCAA | GATTTAGACTAC | ther- | 2a |
| | nuclease | | ACGAAGGGGACTAAAACT | CCAATA | CCCAAAAACGAA | mo- | |
| | valida- | | ATCAACCAATAATAGTCTG | ATAGTC | GGGGACTAAAAC | nu- | |
| | tion | | AATGTCAT | TGAATG | (SEQ ID. | clease | |
| | LwaCas13 | | (SEQ ID. NO: 3147) | TCAT | NO: 3149) | | |
| | a 1 (top | | | (SEQ ID. | | | |
| | pre- | | | NO: | | | |
| | dicted) | | | 3148) | | | |
| 2c | thermo- | LwaCas13a | GATTTAGACTACCCCAAAA | TTGCTT | GATTTAGACTAC | ther- | 2a |
| | nuclease | | ACGAAGGGGACTAAAACTT | CAGGA | CCCAAAAACGAA | mo- | |
| | valida- | | GCTTCAGGACCATATTTCT | CCATAT | GGGGACTAAAAC | nu- | |
| | tion | | CTACACC | TTCTCT | (SEQ ID. | clease | |
| | LwaCas13 | | (SEQ ID. NO: 3150) | ACACC | NO: 3152) | | |
| | a 20 | | | (SEQ ID. | | | |
| | (bottom | | | NO: | | | |
| | pre- | | | 3151) | | | |
| | dicted) | | | | | | |
| 2c | APML | LwaCas13a | GATTTAGACTACCCCAAAA | GCGCCA | GATTTAGACTAC | APML | 2a |
| | long | | ACGAAGGGGACTAAAACG | CTGGCC | CCCAAAAACGAA | long | |
| | valida- | | CGCCACTGGCCACGTGGTT | ACGTGG | GGGGACTAAAAC | | |
| | tion | | GCTGTTGG | TTGCTG | (SEQ ID. | | |
| | LwaCas13 | | (SEQ ID. NO: 3153) | TTGG | NO: 3155) | | |
| | a 1 (top | | | (SEQ ID. | | | |
| | pre- | | | NO: | | | |
| | dicted) | | | 3154) | | | |
| 2c | APML | LwaCas13a | GATTTAGACTACCCCAAAA | TCCCCG | GATTTAGACTAC | APML | 2a |
| | long | | ACGAAGGGGACTAAAACT | GCGCCA | CCCAAAAACGAA | long | |
| | valida- | | CCCCGGCGCCACTGGCCAC | CTGGCC | GGGGACTAAAAC | | |
| | tion | | GTGGTTGC | ACGTGG | (SEQ ID. | | |
| | LwaCas13 | | (SEQ ID. NO: 3156) | TTGC | NO: 3158) | | |
| | a 18 | | | (SEQ ID. | | | |
| | (bottom | | | NO: | | | |
| | pre- | | | 3157) | | | |
| | dicted) | | | | | | |
| 2c | APML | LwaCas13a | GATTTAGACTACCCCAAAA | TCTCAA | GATTTAGACTAC | APML | 2a |
| | short | | ACGAAGGGGACTAAAACT | TGGCTT | CCCAAAAACGAA | short | |
| | valida- | | CTCAATGGCTTTCCCCTGG | TCCCCT | GGGGACTAAAAC | | |
| | tion | | GTGATGCA | GGGTGA | (SEQ ID. | | |
| | LwaCas13 | | (SEQ ID. NO: 3159) | TGCA | NO: 3161) | | |
| | a 1 (top | | | (SEQ ID. | | | |
| | pre- | | | NO: | | | |
| | dicted) | | | 3160) | | | |
| 2c | APML | LwaCas13a | GATTTAGACTACCCCAAAA | TGGGTC | GATTTAGACTAC | APML | 2a |
| | short | | ACGAAGGGGACTAAAACT | TCAATG | CCCAAAAACGAA | short | |
| | valida- | | GGGTCTCAATGGCTTTCCC | GCTTTC | GGGGACTAAAAC | | |
| | tion | | CTGGGTGA | CCCTGG | (SEQ ID. | | |
| | LwaCas13 | | (SEQ ID. NO: 3162) | GTGA | NO: 3164) | | |
| | a 23 | | | (SEQ ID. | | | |
| | (bottom | | | NO: | | | |
| | pre- | | | 3163) | | | |
| | dicted) | | | | | | |
| 2d | Thermo- | CcaCas13b | caggtgtatcaaccaataatagtct | caggtgtatc | GTTGGAACTGCT | Ther- | 2b |
| | nuclease | | gaatgGTTGGAACTGCTCTCATTTT | aaccaataat | CTCATTTTGGAGG | mo- | |
| | valida- | | GGAGGGTAATCACAAC | agtctgaatg | GTAATCACAAC | nu- | |
| | tion | | (SEQ ID. NO: 3165) | (SEQ ID. | (SEQ ID. | clease | |
| | CcaCas13 | | | NO: | NO: 3167) | | |
| | b 16 | | | 3166) | | | |
| | (top | | | | | | |
| | pre- | | | | | | |
| | dicted) | | | | | | |
| 2d | Thermo- | CcaCas13b | cttcaattttctttgcattttctac | cttcaattttc | GTTGGAACTGCT | Ther- | 2b |
| | nuclease | | catctGTTGGAACTGCTCTCATTTT | tttgcattttc | CTCATTTTGGAGG | mo- | |
| | valida- | | GGAGGGTAATCACAAC | taccatct | GTAATCACAAC | nu- | |
| | tion | | (SEQ ID. NO: 3168) | (SEQ ID. | (SEQ ID. | clease | |
| | CcaCas13 | | | NO: | NO: 3170) | | |
| | b 48 | | | 3169) | | | |
| | (bottom | | | | | | |
| | pre- | | | | | | |
| | dicted) | | | | | | |
| 2d | APML | CcaCas13b | atggctgcctccccggcgccactgg | atggctgcct | GTTGGAACTGCT | APML | 2b |
| | long | | ccacgGTTGGAACTGCTCTCATTTT | ccccggcgc | CTCATTTTGGAGG | long | |
| | valida- | | GGAGGGTAATCACAAC | cactggcca | GTAATCACAAC | | |
| | tion | | (SEQ ID. NO: 3171) | cg (SEQ | (SEQ ID. | | |
| | CcaCas13 | | | ID. NO: | NO: 3173) | | |
| | b 14 | | | 3172) | | | |
| | (top | | | | | | |
| | pre- | | | | | | |
| | dicted) | | | | | | |
| 2d | APML | CcaCas13b | tggctgcctccccggcgccactggc | tggctgcctc | GTTGGAACTGCT | APML | 2b |
| | long | | cacgtGTTGGAACTGCTCTCATTTT | cccggcgcc | CTCATTTTGGAGG | long | |
| | valida- | | GGAGGGTAATCACAAC | actggccac | GTAATCACAAC | | |
| | tion | | (SEQ ID. NO: 3174) | gt(SEQ | (SEQ ID. | | |
| | CcaCas13 | | | ID. NO: | NO: 3176) | | |
| | b 13 | | | 3175) | | | |
| | (bottom | | | | | | |
| | pre- | | | | | | |
| | dicted) | | | | | | |
| 2d | APML | CcaCas13b | cccctgggtgatgcaagagctgagg | cccctgggt | GTTGGAACTGCT | APML | 2b |
| | short | | tcctgGTTGGAACTGCTCTCATTTT | gatgcaaga | CTCATTTTGGAGG | short | |
| | valida- | | GGAGGGTAATCACAAC | gctgaggtc | GTAATCACAAC | | |
| | tion | | (SEQ ID. NO: 3177) | ctg (SEQ | (SEQ ID. | | |
| | CcaCas13 | | | ID. NO: | NO: 3179) | | |
| | b 4 (top | | | 3178) | | | |
| | pre- | | | | | | |
| | dicted) | | | | | | |
| 2d | APML | CcaCas13b | ctcaatggctttcccctgggtgatg | ctcaatggct | GTTGGAACTGCT | APML | 2b |
| | short | | caagaGTTGGAACTGCTCTCATTTT | ttcccctggg | CTCATTTTGGAGG | short | |
| | valida- | | GGAGGGTAATCACAAC | tgatgcaag | GTAATCACAAC | | |
| | tion | | (SEQ ID. NO: 3180) | a (SEQ | (SEQ ID. | | |
| | CcaCas13 | | | ID. NO: | NO: 3182) | | |
| | b 16 | | | 3181) | | | |
| | (bottom | | | | | | |
| | pre- | | | | | | |
| | dicted) | | | | | | |
| 2e | thermo- | LwaCas13a | GATTTAGACTACCCCAAAA | TATCAA | GATTTAGACTAC | ther- | 2a |
| | nuclease | | ACGAAGGGGACTAAAACT | CCAATA | CCCAAAAACGAA | mo- | |
| | valida- | | ATCAACCAATAATAGTCTG | ATAGTC | GGGGACTAAAAC | nu- | |
| | tion | | AATGTCAT | TGAATG | (SEQ ID. | clease | |
| | LwaCas13 | | (SEQ ID. NO: 3183) | TCAT | NO: 3185) | | |
| | a 1 (top | | | (SEQ ID. | | | |
| | pre- | | | NO: | | | |
| | dicted) | | | 3184) | | | |
| 2e | thermo- | LwaCas13a | GATTTAGACTACCCCAAAA | TTGCTT | GATTTAGACTAC | ther- | 2a |
| | nuclease | | ACGAAGGGGACTAAAACTT | CAGGA | CCCAAAAACGAA | mo- | |
| | valida- | | GCTTCAGGACCATATTTCT | CCATAT | GGGGACTAAAAC | nu- | |
| | tion | | CTACACC | TTCTCT | (SEQ ID. | clease | |
| | LwaCas13 | | (SEQ ID. NO: 3186) | ACACC | NO: 3188) | | |
| | a 20 | | | (SEQ ID. | | | |
| | (bottom | | | NO: | | | |
| | pre- | | | 3187) | | | |
| | dicted) | | | | | | |
| 2e | APML | LwaCas13a | GATTTAGACTACCCCAAAA | GCGCCA | GATTTAGACTAC | APML | 2a |
| | long | | ACGAAGGGGACTAAAACG | CTGGCC | CCCAAAAACGAA | long | |
| | valida- | | CGCCACTGGCCACGTGGTT | ACGTGG | GGGGACTAAAAC | | |
| | tion | | GCTGTTGG | TTGCTG | (SEQ ID. | | |
| | LwaCas13 | | (SEQ ID. NO: 3189) | TTGG | NO: 3191) | | |
| | a 1 (top | | | (SEQ ID. | | | |
| | pre- | | | NO: | | | |
| | dicted) | | | 3190) | | | |
| 2e | APML | LwaCas13a | GATTTAGACTACCCCAAAA | TCCCCG | GATTTAGACTAC | APML | 2a |
| | long | | ACGAAGGGGACTAAAACT | GCGCCA | CCCAAAAACGAA | long | |
| | valida- | | CCCCGGCGCCACTGGCCAC | CTGGCC | GGGGACTAAAAC | | |
| | tion | | GTGGTTGC | ACGTGG | (SEQ ID. | | |
| | LwaCas13 | | (SEQ ID. NO: 3192) | TTGC | NO: 3194) | | |
| | a 18 | | | (SEQ ID. | | | |
| | (bottom | | | NO: | | | |
| | pre- | | | 3193) | | | |
| | dicted) | | | | | | |
| 2e | APML | LwaCas13a | GATTTAGACTACCCCAAAA | TCTCAA | GATTTAGACTAC | APML | 2a |
| | short | | ACGAAGGGGACTAAAACT | TGGCTT | CCCAAAAACGAA | short | |
| | valida- | | CTCAATGGCTTTCCCCTGG | TCCCCT | GGGGACTAAAAC | | |
| | tion | | GTGATGCA | GGGTGA | (SEQ ID. | | |
| | LwaCas13 | | (SEQ ID. NO: 3195) | TGCA | NO: 3197) | | |
| | a 1 (top | | | (SEQ ID. | | | |
| | pre- | | | NO: | | | |
| | dicted) | | | 3196) | | | |
| 2e | APML | LwaCas13a | GATTTAGACTACCCCAAAA | TGGGTC | GATTTAGACTAC | APML | 2a |
| | short | | ACGAAGGGGACTAAAACT | TCAATG | CCCAAAAACGAA | short | |
| | valida- | | GGGTCTCAATGGCTTTCCC | GCTTTC | GGGGACTAAAAC | | |
| | tion | | CTGGGTGA | CCCTGG | (SEQ ID. | | |
| | LwaCas13 | | (SEQ ID. NO: 3198) | GTGA | NO: 3200) | | |
| | a 23 | | | (SEQ ID. | | | |
| | (bottom | | | NO: | | | |
| | pre- | | | 3199) | | | |
| | dicted) | | | | | | |
| 2f | Thermo- | CcaCas13b | caggtgtatcaaccaataatagtct | caggtgtatc | GTTGGAACTGCT | Ther- | 2b |
| | nuclease | | gaatgGTTGGAACTGCTCTCATTTT | aaccaataat | CTCATTTTGGAGG | mo- | |
| | valida- | | GGAGGGTAATCACAAC | agtctgaatg | GTAATCACAAC | nu- | |
| | tion | | (SEQ ID. NO: 3201) | (SEQ ID. | (SEQ ID. | clease | |
| | CcaCas13 | | | NO: | NO: 3203) | | |
| | b 16 | | | 3202) | | | |
| | (top | | | | | | |
| | pre- | | | | | | |
| | dicted) | | | | | | |
| 2f | Thermo- | CcaCas13b | cttcaattttctttgcattttctac | cttcaattttc | GTTGGAACTGCT | Ther- | 2b |
| | nuclease | | catctGTTGGAACTGCTCTCATTTT | tttgcattttc | CTCATTTTGGAGG | mo- | |
| | valida- | | GGAGGGTAATCACAAC | taccatct | GTAATCACAAC | nu- | |
| | tion | | (SEQ ID. NO: 3204) | (SEQ ID. | (SEQ ID. | clease | |
| | CcaCas13 | | | NO: | NO: 3206) | | |
| | b 48 | | | 3205) | | | |
| | (bottom | | | | | | |
| | pre- | | | | | | |
| | dicted) | | | | | | |
| 2f | APML | CcaCas13b | atggctgcctccccggcgccactgg | atggctgcct | GTTGGAACTGCT | APML | 2b |
| | long | | ccacgGTTGGAACTGCTCTCATTTT | ccccggcgc | CTCATTTTGGAGG | long | |
| | valida- | | GGAGGGTAATCACAAC | cactggcca | GTAATCACAAC | | |
| | tion | | (SEQ ID. NO: 3207) | cg (SEQ | (SEQ ID. | | |
| | CcaCas13 | | | ID. NO: | NO: 3209) | | |
| | b 14 | | | 3208) | | | |
| | (top | | | | | | |
| | pre- | | | | | | |
| | dicted) | | | | | | |
| 2f | APML | CcaCas13b | tggctgcctccccggcgccactggc | tggctgcctc | GTTGGAACTGCT | APML | 2b |
| | long | | cacgtGTTGGAACTGCTCTCATTTT | cccggcgcc | CTCATTTTGGAGG | long | |
| | valida- | | GGAGGGTAATCACAAC | actggccac | GTAATCACAAC | | |
| | tion | | (SEQ ID. NO: 3210) | gt(SEQ | (SEQ ID. | | |
| | CcaCas13 | | | ID. NO: | NO: 3212) | | |
| | b 13 | | | 3211) | | | |
| | (bottom | | | | | | |
| | pre- | | | | | | |
| | dicted) | | | | | | |
| 2f | APML | CcaCas13b | cccctgggtgatgcaagagctgagg | cccctgggt | GTTGGAACTGCT | APML | 2b |
| | short | | tcctgGTTGGAACTGCTCTCATTTT | gatgcaaga | CTCATTTTGGAGG | short | |
| | valida- | | GGAGGGTAATCACAAC | gctgaggtc | GTAATCACAAC | | |
| | tion | | (SEQ ID. NO: 3213) | ctg (SEQ | (SEQ ID. | | |
| | CcaCas13 | | | ID. NO: | NO: 3215) | | |
| | b 4 (top | | | 3214) | | | |
| | pre- | | | | | | |
| | dicted) | | | | | | |
| 2f | APML | CcaCas13b | ctcaatggctttcccctgggtgatg | ctcaatggct | GTTGGAACTGCT | APML | 2b |
| | short | | caagaGTTGGAACTGCTCTCATTTT | ttcccctggg | CTCATTTTGGAGG | short | |
| | valida- | | GGAGGGTAATCACAAC | tgatgcaag | GTAATCACAAC | | |
| | tion | | (SEQ ID. NO: 3216) | a (SEQ | (SEQ ID. | | |
| | CcaCas13 | | | ID. NO: | NO: 3218) | | |
| | b 16 | | | 3217) | | | |
| | (bottom | | | | | | |
| | pre- | | | | | | |
| | dicted) | | | | | | |
| 3b | Acyl- | LwaCas13a | GATTTAGACTACCCCAAAA | GCACGC | GATTTAGACTAC | Acyl- | 3b |
| | trans- | | ACGAAGGGGACTAAAACgca | TGGAGG | CCCAAAAACGAA | trans- | |
| | ferase | | cgctggaggggtcgagcacgctcac | GGTCGA | GGGGACTAAAAC | ferase | |
| | LwaCas13 | | (SEQ ID. NO: 3219) | GCACGC | (SEQ ID. | | |
| | a top | | | TCAC | NO: 3221) | | |
| | pre- | | | (SEQ ID. | | | |
| | dicted | | | NO: | | | |
| | crRNA | | | 3220) | | | |
| 3c | Acyl- | LwaCas13a | GATTTAGACTACCCCAAAA | CATCGC | GATTTAGACTAC | Acyl- | 3c |
| | trans- | | ACGAAGGGGACTAAAACcat | AGAGC | CCCAAAAACGAA | trans- | |
| | ferase | | cgcagagcacgctggaggggtcgag | ACGCTG | GGGGACTAAAAC | ferase | |
| | LwaCas13 | | (SEQ ID. NO: 3222) | GAGGG | (SEQ ID. | | |
| | a bottom | | | GTCGAG | NO: 3224) | | |
| | pre- | | | (SEQ ID. | | | |
| | dicted | | | NO: | | | |
| | crRNA | | | 3223) | | | |
| 3d-f | Acyl- | LwaCas13a | GATTTAGACTACCCCAAAA | GCACGC | GATTTAGACTAC | Acyl- | 3b |
| | trans- | | ACGAAGGGGACTAAAACgca | TGGAGG | CCCAAAAACGAA | trans- | |
| | ferase | | cgctggaggggtcgagcacgctcac | GGTCGA | GGGGACTAAAAC | ferase | |
| | LwaCas13 | | (SEQ ID. NO: 3225) | GCACGC | (SEQ ID. | | |
| | a top | | | TCAC | NO: 3227) | | |
| | pre- | | | (SEQ ID. | | | |
| | dicted | | | NO: | | | |
| | crRNA | | | 3226) | | | |
| 3d-f | Acyl- | LwaCas13a | GATTTAGACTACCCCAAAA | CATCGC | GATTTAGACTAC | Acyl- | 3c |
| | trans- | | ACGAAGGGGACTAAAACcat | AGAGC | CCCAAAAACGAA | trans- | |
| | ferase | | cgcagagcacgctggaggggtcgag | ACGCTG | GGGGACTAAAAC | ferase | |
| | LwaCas13 | | (SEQ ID. NO: 3228) | GAGGG | (SEQ ID. | | |
| | a bottom | | | GTCGAG | NO: 3230) | | |
| | pre- | | | (SEQ ID. | | | |
| | dicted | | | NO: | | | |
| | crRNA | | | 3229) | | | |
| 3h | Thermo- | CcaCas13b | caggtgtatcaaccaataatagtct | caggtgtatc | GTTGGAACTGCT | Ther- | 2b |
| | nuclease | | gaatgGTTGGAACTGCTCTCATTTT | aaccaataat | CTCATTTTGGAGG | mo- | |
| | valida- | | GGAGGGTAATCACAAC | agtctgaatg | GTAATCACAAC | nu- | |
| | tion | | (SEQ ID. NO: 3231) | (SEQ ID. | (SEQ ID. | clease | |
| | CcaCas13 | | | NO: | NO: 3233) | | |
| | b 16 | | | 3232) | | | |
| | (top | | | | | | |
| | pre- | | | | | | |
| | dicted) | | | | | | |
| 3i | Thermo- | CcaCas13b | cttcaattttctttgcattttctac | cttcaattttc | GTTGGAACTGCT | Ther- | 2b |
| | nuclease | | catctGTTGGAACTGCTCTCATTTT | tttgcattttc | CTCATTTTGGAGG | mo- | |
| | valida- | | GGAGGGTAATCACAAC | taccatct | GTAATCACAAC | nu- | |
| | tion | | (SEQ ID. NO: 3234) | (SEQ ID. | (SEQ ID. | clease | |
| | CcaCas13 | | | NO: | NO: 3236) | | |
| | b 48 | | | 3235) | | | |
| | (bottom | | | | | | |
| | pre- | | | | | | |
| | dicted) | | | | | | |
| 3j-l | Thermo- | CcaCas13b | caggtgtatcaaccaataatagtct | caggtgtatc | GTTGGAACTGCT | Ther- | 2b |
| | nuclease | | gaatgGTTGGAACTGCTCTCATTTT | aaccaataat | CTCATTTTGGAGG | mo- | |
| | valida- | | GGAGGGTAATCACAAC | agtctgaatg | GTAATCACAAC | nu- | |
| | tion | | (SEQ ID. NO: 3237) | (SEQ ID. | (SEQ ID. | clease | |
| | CcaCas13 | | | NO: | NO: 3239) | | |
| | b 16 | | | 3238) | | | |
| | (top | | | | | | |
| | pre- | | | | | | |
| | dicted) | | | | | | |
| 3j-l | Thermo- | CcaCas13b | cttcaattttctttgcattttctac | cttcaattttc | GTTGGAACTGCT | Ther- | 2b |
| | nuclease | | catctGTTGGAACTGCTCTCATTTT | tttgcattttc | CTCATTTTGGAGG | mo- | |
| | valida- | | GGAGGGTAATCACAAC | taccatct | GTAATCACAAC | nu- | |
| | tion | | (SEQ ID. NO: 3240) | (SEQ ID. | (SEQ ID. | clease | |
| | CcaCas13 | | | NO: | NO: 3242) | | |
| | b 48 | | | 3241) | | | |
| | (bottom | | | | | | |
| | pre- | | | | | | |
| | dicted) | | | | | | |
| 4b | Ea175 | LwaCas13a | GATTTAGACTACCCCAAAA | AAGATG | GATTTAGACTAC | Ea175 | 4b |
| | LwaCas13 | | ACGAAGGGGACTAAAACA | TGGATT | CCCAAAAACGAA | | |
| | atop | | AGATGTGGATTTTTACATA | TTTACA | GGGGACTAAAAC | | |
| | pre- | | GTAAAAAT | TAGTAA | (SEQ ID. | | |
| | dicted | | (SEQ ID. NO: 3243) | AAAT | NO: 3245) | | |
| | | | | (SEQ ID. | | | |
| | | | | NO: | | | |
| | | | | 3244) | | | |
| 4b | Thermo- | CcaCas13b | caggtgtatc aaccaataatagtc | caggtgtatc | GTTGGAACTGCT | Ther- | 2b |
| | nuclease | | tgaatgGTTGGAACTGCTCTCATTT | aaccaataat | CTCATTTTGGAGG | mo- | |
| | valida- | | TGGAGGGTAATCACAAC | agtctgaatg | GTAATCACAAC | nu- | |
| | tion | | (SEQ ID. NO: 3246) | (SEQ ID. | (SEQ ID. | clease | |
| | CcaCas13 | | | NO: | NO: 3248) | | |
| | b 16 | | | 3247) | | | |
| | (top | | | | | | |
| | pre- | | | | | | |
| | dicted) | | | | | | |
| 4d-e | Ea175 | LwaCas13a | GATTTAGACTACCCCAAAA | AAGATG | GATTTAGACTAC | Ea175 | 4b |
| | LwaCas13 | | ACGAAGGGGACTAAAACA | TGGATT | CCCAAAAACGAA | | |
| | atop | | AGATGTGGATTTTTACATA | TTTACA | GGGGACTAAAAC | | |
| | pre- | | GTAAAAAT | TAGTAA | (SEQ ID. | | |
| | dicted | | (SEQ ID. NO: 3249) | AAAT | NO: 3251) | | |
| | | | | (SEQ ID. | | | |
| | | | | NO: | | | |
| | | | | 3250) | | | |
| 4d-e | Thermo- | CcaCas13b | caggtgtatc aaccaataatagtc | caggtgtatc | GTTGGAACTGCT | Ther- | 2b |
| | nuclease | | tgaatgGTTGGAACTGCTCTCATTT | aaccaataat | CTCATTTTGGAGG | mo- | |
| | valida- | | TGGAGGGTAATCACAAC | agtctgaatg | GTAATCACAAC | nu- | |
| | tion | | (SEQ ID. NO: 3252) | (SEQ ID. | (SEQ ID. | clease | |
| | CcaCas13 | | | NO: | NO: 3254) | | |
| | b 16 | | | 3253) | | | |
| | (top | | | | | | |
| | pre- | | | | | | |
| | dicted) | | | | | | |
| 8a-c | Ea175 | LwaCas13a | GATTTAGACTACCCCAAAA | AAGATG | GATTTAGACTAC | Ea175 | 4b |
| | LwaCas13 | | ACGAAGGGGACTAAAACA | TGGATT | CCCAAAAACGAA | | |
| | atop | | AGATGTGGATTTTTACATA | TTTACA | GGGGACTAAAAC | | |
| | pre- | | GTAAAAAT | TAGTAA | (SEQ ID. | | |
| | dicted | | (SEQ ID. NO: 3255) | AAAT | NO: 3257) | | |
| | | | | (SEQ ID. | | | |
| | | | | NO: | | | |
| | | | | 3256) | | | |
| 8d-f | Ea81 | LwaCas13a | GATTTAGACTACCCCAAAA | ATTTCT | GATTTAGACTAC | Ea81 | 8d |
| | LwaCas13 | | ACGAAGGGGACTAAAACA | AGAATT | CCCAAAAACGAA | | |
| | atop | | TTTCTAGAATTGAAGGAAT | GAAGG | GGGGACTAAAAC | | |
| | pre- | | TAAACCAA | AATTAA | (SEQ ID. | | |
| | dicted | | (SEQ ID. NO: 3258) | ACCAA | NO: 3260) | | |
| | | | | (SEQ ID. | | | |
| | | | | NO: | | | |
| | | | | 3259) | | | |
| 9d-e | Ea175 | LwaCas13a | GATTTAGACTACCCCAAAA | AAGATG | GATTTAGACTAC | Ea175 | 4b |
| | LwaCas13 | | ACGAAGGGGACTAAAACA | TGGATT | CCCAAAAACGAA | | |
| | atop | | AGATGTGGATTTTTACATA | TTTACA | GGGGACTAAAAC | | |
| | pre- | | GTAAAAAT | TAGTAA | (SEQ ID. | | |
| | dicted | | (SEQ ID. NO: 3261) | AAAT | NO: 3263) | | |
| | | | | (SEQ ID. | | | |
| | | | | NO: | | | |
| | | | | 3262) | | | |
| 10b- | Lectin | LwaCas13a | GATTTAGACTACCCCAAAA | ggggtggag | GATTTAGACTAC | Lectin | 10b |
| c | LwaCas13 | | ACGAAGGGGACTAAAACggg | tagagggcg | CCCAAAAACGAA | | |
| | a crRNA | | gtggagtagagggcgcgaccaagag | cgaccaaga | GGGGACTAAAAC | | |
| | | | (SEQ ID. NO: 3264) | g(SEQ | (SEQ ID. | | |
| | | | | ID. NO: | NO: 3266) | | |
| | | | | 3265) | | | |
| 10b- | ssDN1 | CcaCas13b | acgccaagcttgcatgcctgcaggt | acgccaagc | GTTGGAACTGCT | ssDNA | 10b |
| c | CcaCas13 | | cgagtGTTGGAACTGCTCTCATTTT | ttgcatgcct | CTCATTTTGGAGG | 1 | |
| | b crRNA | | GGAGGGTAATCACAAC | gcaggtcga | GTAATCACAAC | | |
| | | | (SEQ ID. NO: 3267) | gt(SEQ | (SEQ ID. | | |
| | | | | ID. NO: | NO: 3269) | | |
| | | | | 3268) | | | |
| 10e- | Zika | LwaCas13a | GATTTAGACTACCCCAAAA | actccctaga | GATTTAGACTAC | Zika | 10e |
| f | LwaCas13 | | ACGAAGGGGACTAAAACact | accacgaca | CCCAAAAACGAA | | |
| | a crRNA | | ccctagaaccacgacagtttgcctt | gtttgcctt | GGGGACTAAAAC | | |
| | | | (SEQ ID. NO: 3270) | (SEQ ID. | (SEQ ID. | | |
| | | | | NO: | NO: 3272) | | |
| | | | | 3271) | | | |
| 10e- | Dengue | CcaCas13b | tttgcttctgtccagtgagcatggt | tttgcttctgt | GTTGGAACTGCT | Dengue | 10e |
| f | CcaCas13 | | cTttcgGTGGAACTGCTCTCATTTT | ccagtgagc | CTCATTTTGGAGG | | |
| | b crRNA | | GGAGGGTAATCACAAC | atggtcttcg | GTAATCACAAC | | |
| | | | (SEQ ID. NO: 3273) | (SEQ ID. | (SEQ ID. | | |
| | | | | NO: | NO: 3275) | | |
| | | | | 3274) | | | |
| 10e- | ssDNA1 | AsCas12a | TAATTTCTACTCTTGTAGAT | ctgtgtttatc | TAATTTCTACTCT | ssDNA | 10e |
| f | AsCas12a | | ctgtgtttatccgctcacaa | cgctcacaa | TGTAGAT | 1 | |
| | crRNA | | (SEQ ID. NO: 3276) | (SEQ ID. | (SEQ ID. | | |
| | | | | NO: | NO: 3278) | | |
| | | | | 3277) | | | |

TABLE 2
Target sequences used in this study

| | | | DNA/ |
|---|---|---|---|
| FIG. | Name | Target sequence | RNA |
| 1b | Ebola | attcgcagtgaagagttgtctttcacagttgtatcaaacggagccaaaaacatcagtggtcag | RNA |
| | | agtccggcgcgaacttcttccgacccagggaccaacacaacaactgaagaccacaaaatcatg | |
| | | gcttcagaaaattcctctgcaatggttcaagtgcacagtcaa (SEQ ID. NO: 3279) | |
| 1b | Zika | gacaccggaactccacactggaacaacaaagaagcactggtagagttcaaggacgcacatgcc | RNA |
| | | aaaaggcaaactgtcgtggttctagggagtcaagaaggagcagttcacacggcccttgctgga | |
| | | gctctggaggctgagatggatggtgcaaagggaaggctgtcctctggc (SEQ ID. NO: | |
| | | 3280) | |
| 2a-f | Thermo- | agcgattgatggtgatactgttaaattaatgtacaaaggtcaaccaatgacattcagactatt | RNA |
| | nuclease | attggttgatacacctgaaacaaagcatcctaaaaaaggtgtagagaaatatggtcctgaagc | |
| | | aagtgcatttacgaaaaagatggtagaaaatgcaaagaaaattgaag (SEQ ID. NO: | |
| | | 3281) | |
| 2a-f | APML | cacctggatggaccgcctagccccaggagccccgtcataggaagtgaggtcttcctgcccaac | RNA |
| | long | agcaaccacgtggccagtggcgccggggaggcagccattgagacccagagcagcagttctgaa | |
| | | gagatagtgcccagccctccctcgccaccccctctaccccgcatctaca (SEQ ID. NO: | |
| | | 3282) | |
| 2a-f | APML | ggaggagccccagagcctgcaagctgccgtgcgcaccgatggcttcgacgagttcaaggtgcg | RNA |
| | short | cctgcaggacctcagctcttgcatcacccaggggaaagccattgagacccagagcagcagttc | |
| | | tgaagagatagtgcccagccctccctcgccaccccctctaccccgcatc (SEQ ID. NO: | |
| | | 3283) | |
| 3b-f | Acyle- | gtcgggcgcgcacgttttcccttcgctgagcacgctgcgcgcgtcgcctacgtgaatgcgctg | DNA |
| | trans- | ttcgatgcgttggccgaaggcaacccgcgggtgagcgtgctcgacccctccagcgtgctctgc | |
| | ferase | gatggcctggattgtttcgccgaacgtgatggctggtcgctgtacatgg (SEQ ID. NO: | |
| | | 3284) | |
| 3h-l | Thermo- | agcgattgatggtgatactgttaaattaatgtacaaaggtcaaccaatgacattcagactatt | DNA |
| | nuclease | attggttgatacacctgaaacaaagcatcctaaaaaaggtgtagagaaatatggtcctgaagc | |
| | | aagtgcatttacgaaaaagatggtagaaaatgcaaagaaaattgaag (SEQ ID. NO: | |
| | | 3285) | |
| 4b | Thermo- | agcgattgatggtgatactgttaaattaatgtacaaaggtcaaccaatgacattcagactatt | DNA |
| | nuclease | attggttgatacacctgaaacaaagcatcctaaaaaaggtgtagagaaatatggtcctgaagc | |
| | | aagtgcatttacgaaaaagatggtagaaaatgcaaagaaaattgaag (SEQ ID. NO: | |
| | | 3286) | |
| 4b | Ea175 | GGCCAGTTTGAATAAGACAATGAATTATTTTTACTATGTAAAAATCCACATCTTTCACATTTC | DNA |
| | | CATTCTTGTGTTTCA (SEQ ID. NO: 3287) | |
| 4d-e | Thermo- | agcgattgatggtgatactgttaaattaatgtacaaaggtcaaccaatgacattcagactatt | DNA |
| | nuclease | attggttgatacacctgaaacaaagcatcctaaaaaaggtgtagagaaatatggtcctgaagc | |
| | | aagtgcatttacgaaaaagatggtagaaaatgcaaagaaaattgaag (SEQ ID. NO: | |
| | | 3288) | |
| 4d-e | Ea175 | GGCCAGTTTGAATAAGACAATGAATTATTTTTACTATGTAAAAATCCACATCTTTCACATTTC | DNA |
| | | CATTCTTGTGTTTCA (SEQ ID. NO: 3289) | |
| 7a | ssRNA 1 | GGCCAGTGAATTCGAGCTCGGTACCCGGGGATCCTCTAGAAATATGGATTACTTGgtAGAACA | RNA |
| | | GCAATCTACTCGACCTGCAGGCATGCAAGCTTGGCGTAATCATGGTCATAGCTGTTTCCTGTG | |
| | | TTTATCCGCTCACAATTCCACACAACATACGAGCCGGAAGCATAAAG (SEQ ID. NO: | |
| | | 3290) | |
| 7a | Thermo- | agcgattgatggtgatactgttaaattaatgtacaaaggtcaaccaatgacattcagactatt | RNA |
| | nuclease | attggttgatacacctgaaacaaagcatcctaaaaaaggtgtagagaaatatggtcctgaagc | |
| | | aagtgcatttacgaaaaagatggtagaaaatgcaaagaaaattgaag (SEQ ID. NO: | |
| | | 3291) | |
| 7a | Dengue | agtacatattcaggggccaacctctcaacaatgacgaagaccatgctcactggacagaagcaa | RNA |
| | | aaatgctgctggacaacatcaacacaccagaagggattataccagctctctttgaaccagaaa | |
| | | gggagaagtcagccgccatagacggtgaataccgcctgaagggt (SEQ ID. NO: 3292) | |
| 8a-c | Ea175 | GGCCAGTTTGAATAAGACAATGAATTATTTTTACTATGTAAAAATCCACATCTTTCACATTTC | DNA |
| | | CATTCTTGTGTTTCA (SEQ ID. NO: 3293) | |
| 8d-f | Ea81 | ATTGTTACATTGTACACATACATAAGCAACATAAGCATCATTTGGTTTAATTCCTTCAATTCT | DNA |
| | | AGAAATATTTGTTTGATTTTTTACTTCACGCCTACTCAT (SEQ ID. NO: 3294) | |
| 8d-f | Ea175 | GGCCAGTTTGAATAAGACAATGAATTATTTTTACTATGTAAAAATCCACATCTTTCACATTTC | DNA |
| | | CATTCTTGTGTTTCA (SEQ ID. NO: 3295) | |
| 10b-c | Lectin | aagttacaactcaataaggttgacgaaaacggcaccccaaaaccctcgtctcttggtcgcgcc | DNA |
| | | ctctactccacccccatccacatttgggacaaagaaaccggtagcgttgccagcttcgccgct | |
| | | tccttcaacttcaccttctatgcccctgacacaaaaaggcttgcagatgggcttgccttcttt | |
| | | ctcgc (SEQ ID. NO: 3296) | |
| 10b-c | ssDNA 1 | GGCCAGTGAATTCGAGCTCGGTACCCGGGGATCCTCTAGAAATATGGATTACTTGgtAGAACA | DNA |
| | | GCAATCTACTCGACCTGCAGGCATGCAAGCTTGGCGTAATCATGGTCATAGCTGTTTCCTGTG | |
| | | TTTATCCGCTCACAATTCCACACAACATACGAGCCGGAAGCATAAAG (SEQ ID. NO: | |
| | | 3297) | |
| 10e-f | Zika | gacaccggaactccacactggaacaacaaagaagcactggtagagttcaaggacgcacatgcc | RNA |
| | | aaaaggcaaactgtcgtggttctagggagtcaagaaggagcagttcacacggcccttgctgga | |
| | | gctctggaggctgagatggatggtgcaaagggaaggctgtcctctggc (SEQ ID. NO: | |
| | | 3298) | |
| 10e-f | Dengue | agtacatattcaggggccaacctctcaacaatgacgaagaccatgctcactggacagaagcaa | RNA |
| | | aaatgctgctggacaacatcaacacaccagaagggattataccagctctctttgaaccagaaa | |
| | | gggagaagtcagccgccatagacggtgaataccgcctgaagggt (SEQ ID. NO: 3299) | |
| 10e-f | ssDNA 1 | GGCCAGTGAATTCGAGCTCGGTACCCGGGGATCCTCTAGAAATATGGATTACTTGgtAGAACA | DNA |
| | | GCAATCTACTCGACCTGCAGGCATGCAAGCTTGGCGTAATCATGGTCATAGCTGTTTCCTGTG | |
| | | TTTATCCGCTCACAATTCCACACAACATACGAGCCGGAAGCATAAAG (SEQ ID. NO: | |
| | | 3300) | |

TABLE 3
RPA primers used in this study

| FIG. | Name | Sequence | Target |
|---|---|---|---|
| 3b | RPA | gaaatTAATACGACTCACTATAGGGCTAC | acyl- |
| | Acyltransferase F | GTGAATGCGCTGTTCGATG (SEQ ID. | transferase |
| | with T7 | NO: 3301) | |
| 3b | RPA | CATCACGTTCGGCGAAACAATCCAG | acyl- |
| | Acyltransferase R | (SEQ ID. NO: 3302) | transferase |
| 3c | RPA | gaaatTAATACGACTCACTATAGGGCTAC | acyl- |
| | Acyltransferase F | GTGAATGCGCTGTTCGATG (SEQ ID. | transferase |
| | with T7 | NO: 3303) | |
| 3c | RPA | CATCACGTTCGGCGAAACAATCCAG | acyl- |
| | Acyltransferase R | (SEQ ID. NO: 3304) | transferase |
| 3e | RPA | gaaatTAATACGACTCACTATAGGGCTAC | acyl- |
| | Acyltransferase F | GTGAATGCGCTGTTCGATG (SEQ ID. | transferase |
| | with T7 | NO: 3305) | |
| 3e | RPA | CATCACGTTCGGCGAAACAATCCAG | acyl- |
| | Acyltransferase R | (SEQ ID. NO: 3306) | transferase |
| 3h | RPA | gaaatTAATACGACTCACTATAGGGTGTA | thermo- |
| | Thermonuclease F | CAAAGGTCAACCAATGACATTCAG | nuclease |
| | with T7 | (SEQ ID. NO: 3307) | thermo- |
| 3h | RPA | TGCACTTGCTTCAGGACCATATTTC | nuclease |
| | Thermonuclease R | (SEQ ID. NO: 3308) | |
| 3i | RPA | gaaatTAATACGACTCACTATAGGGTGTA | thermo- |
| | Thermonuclease F | CAAAGGTCAACCAATGACATTCAG | nuclease |
| | with T7 | (SEQ ID. NO: 3309) | |
| 3i | RPA | TGCACTTGCTTCAGGACCATATTTC | thermo- |
| | Thermonuclease R | (SEQ ID. NO: 3310) | nuclease |
| 3k | RPA | gaaatTAATACGACTCACTATAGGGTGTA | thermo- |
| | Thermonuclease F | CAAAGGTCAACCAATGACATTCAG | nuclease |
| | with T7 | (SEQ ID. NO: 3311) | |
| 3k | RPA | TGCACTTGCTTCAGGACCATATTTC | thermo- |
| | Thermonuclease R | (SEQ ID. NO: 3312) | nuclease |
| 4b | Multiplexing RPA | gaaatTAATACGACTCACTATAGGGAGCG | thermo- |
| | Thermonuclease F | ATTGATGGTGATACTGTTAAA (SEQ | nuclease |
| | with T7 | ID. NO: 3313) | |
| 4b | Multiplexing RPA | TCGTAAATGCACTTGCTTCAGGACC | thermo- |
| | Thermonuclease R | (SEQ ID. NO: 3314) | nuclease |
| 4b | RPA Ea175 F with | gaaattaatacgactcactatagggGGCC | Ea175 |
| | T7 | AGTTTGAATAAGACAATG (SEQ ID. | |
| | | NO: 3315) | |
| 4b | RPA Ea175 R | GGCCAGTTTGAATAAGACAATG (SEQ | Ea175 |
| | | ID. NO: 3316) | |
| 4d | Multiplexing RPA | gaaatTAATACGACTCACTATAGGGAGCG | thermo- |
| | Thermonuclease F | ATTGATGGTGATACTGTTAAA (SEQ | nuclease |
| | with T7 | ID. NO: 3317) | |
| 4d | Multiplexing RPA | TCGTAAATGCACTTGCTTCAGGACC | thermo- |
| | Thermonuclease R | (SEQ ID. NO: 3318) | nuclease |
| 4d | RPA Ea175 F with | gaaattaatacgactcactatagggGGCC | Ea175 |
| | T7 | AGTTTGAATAAGACAATG (SEQ ID. | |
| | | NO: 3319) | |
| 4d | RPA Ea175 R | GGCCAGTTTGAATAAGACAATG (SEQ | Ea175 |
| | | ID. NO: 3320) | |
| 4e | Multiplexing RPA | gaaatTAATACGACTCACTATAGGGAGCG | thermo- |
| | Thermonuclease F | ATTGATGGTGATACTGTTAAA (SEQ | nuclease |
| | with T7 | ID. NO: 3321) | |
| 4e | Multiplexing RPA | TCGTAAATGCACTTGCTTCAGGACC | thermo- |
| | Thermonuclease R | (SEQ ID. NO: 3322) | nuclease |
| 4e | RPA Ea175 F with | gaaattaatacgactcactatagggGGCC | Ea175 |
| | T7 | AGTTTGAATAAGACAATG (SEQ ID. | |
| | | NO: 3323) | |
| 4e | RPA Ea175 R | GGCCAGTTTGAATAAGACAATG (SEQ | Ea175 |
| | | ID. NO: 3324) | |
| 8a | RPA Ea175 F with | gaaattaatacgactcactatagggGGCC | Ea175 |
| | T7 | AGTTTGAATAAGACAATG (SEQ ID. | |
| | | NO: 3325) | |
| 8a | RPA Ea175 R | GGCCAGTTTGAATAAGACAATG (SEQ | Ea175 |
| | | ID. NO: 3326) | |
| 8c | RPA Ea175 F with | gaaattaatacgactcactatagggGGCC | Ea175 |
| | T7 | AGTTTGAATAAGACAATG (SEQ ID. | |
| | | NO: 3327) | |
| 8c | RPA Ea175 R | GGCCAGTTTGAATAAGACAATG (SEQ | Ea175 |
| | | ID. NO: 3328) | |
| 8d | RPA Ea81 F with | gaaattaatacgactcactatagggATTG | Ea81 |
| | T7 | TTACATTGTACACATACA (SEQ ID. | |
| | | NO: 3329) | |
| 8d | RPA Ea81 R | ATTGTTACATTGTACACATACA (SEQ | Ea81 |
| | | ID. NO: 3330) | |
| 8f | RPA Ea81 F with | gaaattaatacgactcactatagggATTG | Ea81 |
| | T7 | TTACATTGTACACATACA (SEQ ID. | |
| | | NO: 3331) | |
| 8f | RPA Ea81 R | ATTGTTACATTGTACACATACA (SEQ | Ea81 |
| | | ID. NO: 3332) | |
| S3e | RPA Ea175 F with | gaaattaatacgactcactatagggGGCC | Ea175 |
| | T7 | AGTTTGAATAAGACAATG (SEQ ID. | |
| | | NO: 3333) | |
| S3e | RPA Ea175 R | TGAAACACAAGAATGGAAATGT (SEQ | Ea175 |
| | | ID. NO: 3334) | |
| 10b | RPA ssDNA1 F with | gaaattaatacgactcactatagggGATC | ssDNA1 |
| | T7 | CTCTAGAAATATGGATTACTTGGTAGAAC | |
| | | AG (SEQ ID. NO: 3335) | |
| 10b | RPA ssDNA1 R | GATAAACACAGGAAACAGCTATGACCATG | ssDNA1 |
| | | ATTACG (SEQ ID. NO: 3336) | |
| 10b | RPA lectin F with | gaaatTAATACGACTCACTATAGGGTCAA | Lectin |
| | T7 | TAAGGTTGACGAAAACGGCAC (SEQ | |
| | | ID. NO: 3337) | |
| 10b | RPA lectin R | TAGAAGGTGAAGTTGAAGGAAGCGG | Lectin |
| | | (SEQ ID. NO: 3338) | |
| 10c | RPA ssDNA1 F with | gaaattaatacgactcactatagggGATC | ssDNA1 |
| | T7 | CTCTAGAAATATGGATTACTTGGTAGAAC | |
| | | AG (SEQ ID. NO: 3339) | |
| 10c | RPA ssDNA1 R | GATAAACACAGGAAACAGCTATGACCATG | ssDNA1 |
| | | ATTACG (SEQ ID. NO: 3340) | |
| 10c | RPA lectin F with | gaaatTAATACGACTCACTATAGGGTCAA | Lectin |
| | T7 | TAAGGTTGACGAAAACGGCAC (SEQ | |
| | | ID. NO: 3341) | |
| 10c | RPA lectin R | TAGAAGGTGAAGTTGAAGGAAGCGG | Lectin |
| | | (SEQ ID. NO: 3342) | |

TABLE 4
HDA primers used in this study

| FIG. | Name | Sequence | Target |
|---|---|---|---|
| 9e | HDA | gaaattaatacgactcactatagggGG | Ea175 |
| | Ea175 F | CCAGTTTGAATAAGACAATG (SEQ | |
| | with T7 | ID. NO: 3343) | |
| 9e | HDA | TGAAACACAAGAATGGAAATGT (SEQ | Ea175 |
| | Ea175 R | ID. NO: 3344) | |

TABLE 5
Reporter sequences used in this study

| | | | Fluoro- | Antigen/ | Compatible |
|---|---|---|---|---|---|
| FIG. | Name | Sequence | phore | quencher | enzyme |
| 1b | Rnase Alert v2 | N/A | N/A | N/A | LwaCasl3a/ |
| | | | | | CcaCasl3b |
| 2a | Rnase Alert v2 | N/A | N/A | N/A | LwaCasl3a/ |
| | | | | | CcaCasl3b |
| 2b | Rnase Alert v2 | N/A | N/A | N/A | LwaCasl3a/ |
| | | | | | CcaCasl3b |
| 2c | Rnase Alert v2 | N/A | N/A | N/A | LwaCasl3a/ |
| | | | | | CcaCasl3b |
| 2d | Rnase Alert v2 | N/A | N/A | N/A | LwaCasl3a/ |
| | | | | | CcaCasl3b |
| 2e | Single-plex lateral | /56- | FAM | Biotin | LwaCas13a/ |
| | flow reporter | FAM/rUrUrUrUrUrU/ | | | CcaCasl3b |
| | | 3Bio/ (SEQ ID. | | | |
| | | NO: 3345) | | | |
| 2f | Single-plex lateral | /56- | FAM | Biotin | LwaCas13a/ |
| | flow reporter | FAM/rUrUrUrUrUrU/ | | | CcaCasl3b |
| | | 3Bio/ (SEQ ID. | | | |
| | | NO: 3345) | | | |
| 3b | Rnase Alert v2 | N/A | N/A | N/A | LwaCas13a/ |
| | | | | | CcaCasl3b |
| 3c | Rnase Alert v2 | N/A | N/A | N/A | LwaCas13a/ |
| | | | | | CcaCasl3b |
| 3e | Single-plex lateral | /56- | FAM | Biotin | LwaCas13a/ |
| | flow reporter | FAM/rUrUrUrUrU/ | | | CcaCasl3b |
| | | 3Bio/ (SEQ ID. | | | |
| | | NO: 3345) | | | |
| 3h | Poly-U reporter | /56- | FAM | Iowa | LwaCas13a/ |
| | | FAM/rUrUrUrUrUrU | | Black FQ | CcaCasl3b |
| | | 3IABkFQ/ (SEQ ID. | | | |
| | | NO: 3346) | | | |
| 3i | Poly-U reporter | /56- | FAM | Iowa | LwaCas13a/ |
| | | FAM/rUrUrUrUrU/ | | Black FQ | CcaCasl3b |
| | | 3IABkFQ/ (SEQ ID. | | | |
| | | NO: 3346) | | | |
| 3k | Single-plex lateral | /56- | FAM | Biotin | LwaCas13a/ |
| | flow reporter | FAM/rUrUrUrUrUrU/ | | | CcaCasl3b |
| | | 3Bio/ (SEQ ID. | | | |
| | | NO: 3345) | | | |
| 4b | LwaCas 13a | /56- | FAM | Iowa | LwaCas13a |
| | Fluorescence | FAM/TArArUGC/ | | Black FQ | |
| | reporter | 3IABkFQ/ | | | |
| 4b | CcaCasl3b | /5HEX/TArUrAGC/ | HEX | Iowa | CcaCasl3b |
| | Fluorescence | 3IABkFQ/ | | Black FQ | |
| | reporter | | | | |
| 4d | LwaCas13a Lateral | /5TYE665/T*A*rArU | TYE 665 | AlexaFlu | LwaCas13a |
| | Flow reporter | G*C*/3AlexF488N/ | | or 488 | |
| 4d | CcaCasl3b Lateral | /5TYE665/T*A*rUrA | TYE 665 | FAM | CcaCasl3b |
| | Flow reporter | G*C*/36-FAM/ | | | |
| 4e | LwaCas13a Lateral | /5TYE665/T*A*rArU | TYE 665 | AlexaFlu | LwaCas13a |
| | Flow reporter | G*C*/3AlexF488N/ | | or 488 | |
| 4e | CcaCasl3b Lateral | /5TYE665/T*A*rUrA | TYE 665 | FAM | CcaCasl3b |
| | Flow reporter | G*C*/36-FAM/ | | | |
| 5d | Rnase Alert v2 | N/A | N/A | N/A | LwaCas13a |
| 5e | Single-plex lateral | /56- | FAM | Biotin | LwaCas13a |
| | flow reporter | FAM/rUrUrUrUrUrU/ | | | |
| | | 3Bio/ (SEQ ID. | | | |
| | | NO: 3345) | | | |
| 5f | Single-plex lateral | /56- | FAM | Biotin | LwaCas13a |
| | flow reporter | FAM/rUrUrUrUrUrU/ | | | |
| | | 3Bio/ (SEQ ID. | | | |
| | | NO: 3345) | | | |
| 6b | LwaCas13a Lateral | /5TYE665/T*A*rArU | TYE 665 | AlexaFlu | LwaCas13a |
| | Flow reporter | G*C*/3AlexF488N/ | | or 488 | |
| 6b | CcaCasl3b Lateral | /5TYE665/T*A*rUrA | TYE 665 | FAM | CcaCasl3b |
| | Flow reporter | G*C*/36-FAM/ | | | |
| 6c | LwaCas13a Lateral | /5TYE665/T*A*rArU | TYE 665 | AlexaFlu | LwaCas13a |
| | Flow reporter | G*C*/3AlexF488N/ | | or 488 | |
| 6c | CcaCasl3b Lateral | /5TYE665/T*A*rUrA | TYE 665 | FAM | CcaCasl3b |
| | Flow reporter | G*C*/36-FAM/ | | | |
| 7a | Rnase Alert v2 | N/A | N/A | N/A | LwaCas13a/ |
| | | | | | CcaCasl3b |
| 8a | Rnase Alert v2 | N/A | N/A | N/A | LwaCas13a/ |
| | | | | | CcaCasl3b |
| 8c | Single-plex lateral | /56- | FAM | Biotin | LwaCas13a/ |
| | flow reporter | FAM/rUrUrUrUrUrU/ | | | CcaCasl3b |
| | | 3Bio/ (SEQ ID. | | | |
| | | NO: 3345) | | | |
| 10a | Poly-U reporter | /56- | FAM | Iowa | LwaCas13a/ |
| | | FAM/rUrUrUrUrU/ | | Black FQ | CcaCasl3b |
| | | 3IABkFQ/ (SEQ ID. | | | |
| | | NO: 3346) | | | |
| 10c | Single-plex lateral | /56- | FAM | Biotin | LwaCas13a/ |
| | flow reporter | FAM/rUrUrUrUrUrU/ | | | CcaCasl3b |
| | | 3Bio/ (SEQ ID. | | | |
| | | NO: 3345) | | | |
| 10d | Poly-U reporter | /56- | FAM | Iowa | LwaCas13a/ |
| | | FAM/rUrUrUrUrU/ | | Black FQ | CcaCasl3b |
| | | 3IABkFQ/ | | | |
| 10f | Single-plex lateral | /56- | FAM | Biotin | LwaCas13a/ |
| | flow reporter | FAM/rUrUrUrUrUrU/ | | | CcaCasl3b |
| | | 3Bio/ (SEQ ID. | | | |
| | | NO: 3345) | | | |
| 10b | Helicase reporter | /56- | FAM | N/A | UvrD |
| | FAM | FAM/CAGAGGAAC | | | helicases |
| | | GTCTATCTAACGG | | | |
| | | TTGGTATCTTGAA | | | |
| | | TGCTCAGTCCCTT | | | |
| | | T (SEQ ID. NO: | | | |
| | | 3347) | | | |
| 10b | Helicase reporter | AAAGGGACTGAG | N/A | BHQ-1 | UvrD |
| | BHQ1 | CATTCAAGATACC | | | helicases |
| | | AACCGTTAGATAG | | | |
| | | ACGTTCCTCTG/ | | | |
| | | 3BHQ_1/ (SEQ | | | |
| | | ID. NO: 3348) | | | |
| 10d | Poly-U reporter | /56- | FAM | Iowa | LwaCas13a/ |
| | | FAM/rUrUrUrUrU/ | | Black FQ | CcaCasl3b |
| | | 3IABkFQ/ (SEQ ID. | | | |
| | | NO: 3346) | | | |
| 10e | Poly-U reporter | /56- | FAM | Iowa | LwaCas13a/ |
| | | FAM/rUrUrUrUrU/ | | Black FQ | CcaCasl3b |
| | | 3IABkFQ/ (SEQ ID. | | | |
| | | NO: 3346) | | | |
| 11b | FAM LwaCas 13a | /56- | FAM | Biotin | LwaCas13a |
| | Lateral Flow | FAM/TArArUGC/ | | | |
| | reporter | 3Bio/ | | | |
| 11b | FAM CcaCasl3b | /56- | FAM | DIG | CcaCasl3b |
| | Lateral Flow | FAM/TArUrAGC/ | | | |
| | reporter | 3Dig_N/ | | | |
| 11c | FAM LwaCas 13a | /56- | FAM | Biotin | LwaCas13a |
| | Lateral Flow | FAM/TArArUGC/ | | | |
| | reporter | 3Bio/ | | | |
| 11c | FAM CcaCasl3b | /56- | FAM | DIG | CcaCasl3b |
| | Lateral Flow | FAM/TArUrAGC/ | | | |
| | reporter | 3Dig_N/ | | | |
| 11e | LwaCas13a Lateral | /5TYE665/T*A*rArU | TYE 665 | AlexaFlu | LwaCas13a |
| | Flow reporter | G*C*/3AlexF488N/ | | or 488 | |
| 11e | CcaCasl3b Lateral | /5TYE665/T*A*rUrA | TYE 665 | FAM | CcaCasl3b |
| | Flow reporter | G*C*/36-FAM/ | | | |
| 11e | AsCasl2a Lateral | /5TYE665/CCCCC/ | TYE 665 | DIG | AsCasl2a |
| | Flow reporter | 3Dig_N/ | | | |
| 11f | LwaCas13a Lateral | /5TYE665/T*A*rArU | TYE 665 | AlexaFlu | LwaCas13a |
| | Flow reporter | G*C*/3AlexF488N/ | | or 488 | |
| 11f | CcaCasl3b Lateral | /5TYE665/T*A*rUrA | TYE 665 | FAM | CcaCasl3b |
| | Flow reporter | G*C*/36-FAM/ | | | |
| 11f | AsCasl2a Lateral | /5TYE665/CCCCC/ | TYE 665 | DIG | AsCasl2a |
| | Flow reporter | 3Dig_N/ | | | |
| 12 | Single-plex lateral | /56- | FAM | Biotin | LwaCasl3a |
| | flow reporter | FAM/rUrUrUrUrUrU/ | | | |
| | | 3Bio/ (SEQ ID. | | | |
| | | NO: 3345) | | | |
| 14a | Rnase Alert v2 | N/A | N/A | N/A | LwaCasl3a/ |
| | | | | | CcaCasl3b |
| 14b | Rnase Alert v2 | N/A | N/A | N/A | LwaCasl3a/ |
| | | | | | CcaCasl3b |
| 14c | Rnase Alert v2 | N/A | N/A | N/A | LwaCasl3a/ |
| | | | | | CcaCasl3b |
| 14d | Rnase Alert v2 | N/A | N/A | N/A | LwaCasl3a/ |
| | | | | | CcaCasl3b |

TABLE 6
Cas13 proteins used in this study

| | Protein | | | Accession |
|---|---|---|---|---|
| Abbreviation | name | Strain name | Benchling link | number |
| Lwa | LwaCas13a | Leptotrichia | benchling.com/s/seq- | WP_021746774.1 |
| | | wadei | 66CfLwu7sLMQMbcXe7Ih | |
| Cca | CcaCas13b | Capnocytophaga | benchling.com/s/seq- | WP_013997271 |
| | | canimorsus | BNVzFUQjqSnkYLARxLwE | |

TABLE 7
Helicase proteins used in this study

| | | | | Accession number |
|---|---|---|---|---|
| | Protein | | Superhelicase | (lacks superhelicase |
| Abbreviation | name | Strain name | mutation | mutations) |
| Tte | Tte-UvrD | Thermoanaerobacter | − | AAM23874.1 |
| | | tengcongensis | | |
| Super Tte | Super Tte- | Thermoanaerobacter | + | AAM23874.1 |
| | UvrD | tengcongensis | | |
| Tet | Tet-UvrD | Thermoanaerobacter | − | WP_003870487.1 |
| | | ethanolicus | | |
| Super Tet | Super Tet- | Thermoanaerobacter | + | WP_003870487.1 |
| | UvrD | ethanolicus | | |
| Bsp | Bsp-UvrD | Bacillus sp. FJAT- | − | WP_049660019.1 |
| | | 27231 | | |
| Super Bsp | Super Bsp- | Bacillus sp. FJAT- | + | WP_049660019.1 |
| | UvrD | 27231 | | |
| Bme | Bme-UvrD | Bacillus megaterium | + | WP_034654680.1 |
| Bsi | Bsi-UvrD | Bacillus simplex | + | WP_095390358.1 |
| Pso | Pso-UvrD | Paeniclostridium | + | WP_055343022.1 |
| | | sordellii | | |

TABLE 8
patient samples with source, diagnosis, transcript, variant, and extracted RNA
concentration

| | | | | | | RNA |
|---|---|---|---|---|---|---|
| Sample | | | Chromosomal | Fusion | Transcript | Concentration |
| # | Source | Diagnosis | Translocation | Transcript | Variant | (ng/ul) |
| 1 | PB | APL | t(5; 17) | CLINT1- | N/A | 58.96 |
| | | | | RARA | | |
| 2 | BM | APL | t(15; 17) | PML- | Intron 6 | 124.1 |
| | | | | RARA | | |
| 3 | BM | APL | t(15; 17) | PML- | Intron 6 | 53.3 |
| | | | | RARA | | |
| 4 | BM | APL | t(15; 17) | PML- | Intron 6 | 69.73 |
| | | | | RARA | | |
| 5 | BM | APL | t(15; 17) | PML- | Intron 6 | 469.5 |
| | | | | RARA | | |
| 6 | BM | APL | t(15; 17) | PML- | Exon 6 | 43.96 |
| | | | | RARA | | |
| 7 | BM | APL | t(15; 17) | PML- | Intron 3 | 44.44 |
| | | | | RARA | | |
| 8 | BM | APL | t(15; 17) | PML- | Intron 3 | 25.33 |
| | | | | RARA | | |
| 9 | BM | APL | t(15; 17) | PML- | Intron 3 | 50.18 |
| | | | | RARA | | |
| 10 | BM | APL | t(15; 17) | PML- | Intron 3 | 262.5 |
| | | | | RARA | | |
| 11 | BM | APL | t(15; 17) | PML- | Intron 3 | 191.1 |
| | | | | RARA | | |
| 12 | PB | APL | t(15; 17) | PML- | Intron 3 | 103.6 |
| | | | | RARA | | |
| 13 | BM | ALL | t(9; 22) | BCR- | p210- | 30.98 |
| | | | | ABL | e14a2 | |
| 14 | BM | CML | t(9; 22) | BCR- | p210- | 44.9 |
| | | | | ABL | e14a2 | |
| 15 | BM | CML | t(9; 22) | BCR- | p210- | 225.6 |
| | | | | ABL | e14a2 | |
| 16 | BM | ALL | t(9; 22) | BCR- | p210- | 18.43 |
| | | | | ABL | e13a2 | |
| 17 | BM | CML | t(9; 22) | BCR- | p210- | 38.24 |
| | | | | ABL | e13a2 | |
| 18 | BM | ALL | t(9; 22) | BCR- | p190- | 52.18 |
| | | | | ABL | e1a2 | |
| 19 | BM | ALL | t(9; 22) | BCR- | p190- | 205.6 |
| | | | | ABL | e1a2 | |

TABLE 9
RT-PCR primers for PML-RARA and BCR-ABL fusions

| | | | PCR | | |
|---|---|---|---|---|---|
| Target | Primer | Direction | Round | Sequence (5′-3′) | SEQ ID. NO |
| PML- | PML-A1 | Forward | 1 | CAGTGTACGCCTTCTCCATCA | SEQ ID. NO: |
| RARA | | | | | 3349 |
| | PML-A2 | Forward | 1 | CTGCTGGAGGCTGTGGAC | SEQ ID. NO: |
| | | | | | 3350 |
| | RARA-B | Reverse | 1 | GCTTGTAGATGCGGGGTAGA | SEQ ID. NO: |
| | | | | | 3351 |
| | PML-C1 | Forward | 2 | TCAAGATGGAGTCTGAGGAGG | SEQ ID. NO: |
| | | | | | 3352 |
| | PML-C2 | Forward | 2 | AGCGCGACTACGAGGAGAT | SEQ ID. NO: |
| | | | | | 3353 |
| | RARA-D | Reverse | 2 | CTGCTGCTCTGGGTCTCAAT | SEQ ID. NO: |
| | | | | | 3354 |
| BCR- | BCR-b1- | Forward | 1 | GAAGTGTTTCAGAAGCTTCTC | SEQ ID. NO: |
| ABL | A | | | C | 3355 |
| p210 | | | | | |
| | ABL-a3- | Reverse | 1 | GTTTGGGCTTCACACCATTCC | SEQ ID. NO: |
| | B | | | | 3356 |
| | BCR-b2- | Forward | 2 | CAGATGCTGACCAACTCGTGT | SEQ ID. NO: |
| | C | | | | 3357 |
| | ABL-a3- | Reverse | 2 | TTCCCCATTGTGATTATAGCC | SEQ ID. NO: |
| | D | | | TA | 3358 |
| BCR- | BCR-e1- | Forward | 1 | GACTGCAGCTCCAATGAGAAC | SEQ ID. NO: |
| ABL | A | | | | 3359 |
| p190 | | | | | |
| | ABL-a3- | Reverse | 1 | GTTTGGGCTTCACACCATTCC | SEQ ID. NO: |
| | B | | | | 3360 |
| | BCR-e1- | Forward | 2 | CAGAACTCGCAACAGTCCTTC | SEQ ID. NO: |
| | C | | | | 3361 |
| | ABL-a3- | Reverse | 2 | TTCCCCATTGTGATTATAGCC | SEQ ID. NO: |
| | D | | | TA | 3362 |
| GAPDH | GAPDH- | Forward | 1 | GCACCGTCAAGGCTGAGAAC | SEQ ID. NO: |
| | For | | | | 3363 |
| | GAPDH- | Reverse | 1 | TGGTGAAGACGCCAGTGGA | SEQ ID. NO: |
| | Rev | | | | 3364 |

### REFERENCES

1 Gootenberg, J. S. et al. Multiplexed and portable nucleic acid detection platform with Cas13, Cas12a, and Csm6. Science, doi:10.1126/science.aaq0179 (2018).

2 Gootenberg, J. S. et al. Nucleic acid detection with CRISPR-Cas13a/C2c2. Science 356, 438-442, doi:10.1126/science.aam9321 (2017).

3 Abudayyeh, O. O. et al. C2c2 is a single-component programmable RNA-guided RNA-targeting CRISPR effector. Science 353, aaf5573, doi:10.1126/science.aaf5573 (2016).

4 East-Seletsky, A. et al. Two distinct RNase activities of CRISPR-C2c2 enable guide-RNA processing and RNA detection. Nature 538, 270-273, doi:10.1038/nature19802 (2016).

5 Shmakov, S. et al. Discovery and Functional Characterization of Diverse Class 2 CRISPR-Cas Systems. Mol Cell 60, 385-397, doi:10.1016/j.molce1.2015.10.008 (2015).

6 Smargon, A. A. et al. Cas13b Is a Type VI-B CRISPR-Associated RNA-Guided RNase Differentially Regulated by Accessory Proteins Csx27 and Csx28. Mol Cell 65, 618-630 e617, doi:10.1016/j.molce1.2016.12.023 (2017).

7 Shmakov, S. et al. Diversity and evolution of class 2 CRISPR-Cas systems. Nat Rev Microbiol 15, 169-182, doi:10.1038/nrmicro.2016.184 (2017).

8 Zetsche, B. et al. Cpf1 is a single RNA-guided endonuclease of a class 2 CRISPR-Cas system. Cell 163, 759-771, doi:10.1016/j.ce11.2015.09.038 (2015).

9 Chen, J. S. et al. CRISPR-Cas12a target binding unleashes indiscriminate single-stranded DNase activity. Science, doi:10.1126/science.aar6245 (2018).

10 Myhrvold, C. et al. Field-deployable viral diagnostics using CRISPR-Cas13. Science 360, 444-448, doi:10.1126/science.aas8836 (2018).

11 Li, S. Y. et al. CRISPR-Cas12a-assisted nucleic acid detection. Cell Discov 4, 20, doi:10.1038/101421-018-0028-z (2018).

12 Li, L., Li, S. & Wang, J. CRISPR-Cas12b-assisted nucleic acid detection platform. bioRxiv, 362889, doi:10.1101/362889 (2018).

13 Konermann, S. et al. Transcriptome Engineering with RNA-Targeting Type VI-D CRISPR Effectors. Cell, doi:10.1016/j.ce11.2018.02.033 (2018).

14 Abudayyeh, 0. 0. etal. RNA targeting with CRISPR-Cas13. Nature 550, 280-284, doi:10.1038/nature24049 (2017).

15 Cox, D. B. T. etal. RNA editing with CRISPR-Cas13. Science 358, 1019-1027, doi:10.1126/science.aaq0180 (2017).

16 Zhao, X. et al. A CRISPR-Cas13a system for efficient and specific therapeutic targeting of mutant KRAS for pancreatic cancer treatment. Cancer Lett 431, 171-181, doi:10.1016/j.canlet.2018.05.042 (2018).

17 Hsu, P. D. et al. DNA targeting specificity of RNA-guided Cas9 nucleases. Nat Biotechnol 31, 827-832, doi:10.1038/nbt.2647 (2013).

18 Doench, J. G. et al. Rational design of highly active sgRNAs for CRISPR-Cas9-mediated gene inactivation. Nat Biotechnol 32, 1262-1267, doi:10.1038/nbt.3026 (2014).

19 Piepenburg, O., Williams, C. H., Stemple, D. L. & Armes, N. A. DNA detection using recombination proteins. PLoS Biol 4, e204, doi:10.1371/journal.pbio.0040204 (2006).

20 Vincent, M., Xu, Y. & Kong, H. Helicase-dependent isothermal DNA amplification. EMBO Rep 5, 795-800, doi:10.1038/sj.embor.7400200 (2004).

21 Meiners, M. J., Tahmaseb, K. & Matson, S. W. The UvrD303 hyper-helicase exhibits increased processivity. J Biol Chem 289, 17100-17110, doi:10.1074/jbc.M114.565309 (2014).

22 Ozes, A. R., Feoktistova, K., Avanzino, B. C., Baldwin, E. P. & Fraser, C. S. Real-time fluorescence assays to monitor duplex unwinding and ATPase activities of helicases. Nat Protoc 9, 1645-1661, doi:10.1038/nprot.2014.112 (2014).

23 van der Velden, V. H. et al. Detection of minimal residual disease in hematologic malignancies by real-time quantitative PCR: principles, approaches, and laboratory aspects. Leukemia 17, 1013-1034, doi:10.1038/sj.leu.2402922 (2003).

24 Yan, W. X. et al. Cas13d Is a Compact RNA-Targeting Type VI CRISPR Effector Positively Modulated by a WYL-Domain-Containing Accessory Protein. Mol Cell, doi:10.1016/j.molcel.2018.02.028 (2018).

25 Yan, W. X. et al. Functionally diverse type V CRISPR-Cas systems. Science 363, 88-91, doi:10.1126/science.aav7271 (2019).

26 Ye, J. et al. Primer-BLAST: a tool to design target-specific primers for polymerase chain reaction. BMC Bioinformatics 13, 134, doi:10.1186/1471-2105-13-134 (2012).

27 An, L. et al. Characterization of a thermostable UvrD helicase and its participation in helicase-dependent amplification. J Biol Chem 280, 28952-28958, doi:10.1074/jbc.M503096200 (2005).

28 van Dongen, J. J. et al. Standardized RT-PCR analysis of fusion gene transcripts from chromosome aberrations in acute leukemia for detection of minimal residual disease. Report of the BIOMED-1 Concerted Action: investigation of minimal residual disease in acute leukemia. Leukemia 13, 1901-1928 (1999); doi:/10.1038/sj.leu.2401592 (1999).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A nucleic acid detection system for detecting the presence of one or more cancers in a sample, comprising:
one or more CRISPR system comprising one or more Cas proteins and one or more optimized guide molecules designed to bind to one or more corresponding target molecules of one or more cancer fusion genes; and
one or more RNA-based detection constructs.

2. The system of claim 1, wherein the optimized guide for the target molecule is generated by
(a) pooling a set of guides, the guides produced by tiling guides across the target molecule;
(b) incubating the set of guides with a Cas polypeptide and the target molecule and measuring cleavage activity of each guide in the set;
(c) creating a training model based on the cleavage activity of the set of guides in the incubating step;
(d) predicting highly active guides for the target molecule; and
(e) identifying the optimized guides by incubating the predicted highly active guides with the Cas polypeptide and the target molecule and selecting optimized guides.

3. The system of claim 1, wherein the one or more cancers is selected from acute promyelocytic leukemia (APML), chronic myeloid leukemia (CIVIL), and/or acute lymphoblastic leukemia (ALL).

4. The system of claim 1, wherein the cancer fusion gene is a PML-RARa fusion.

5. The system of claim 1, wherein the Cas protein is LwaCas13a and the guide molecule comprises SEQ ID NO: 2761, 2764, 2767, 2770, 2773, 2776, 2779, 2782, 2785, 2788, 2791, 2794, 2797, 2800, 2803, 2806, 2809, 2812, 2815, 2818, 2821, 2824, 2827, 2830, 2833, 2836, 2839, 2842, 2845, 2848, 2851, 2854, 2857, 2860, 2863, 2866, 2869, 2872, 2875, 2878, 2881, 2884, or 2887.

6. The system of claim 1, wherein the Cas protein is LwaCas13a and the guide molecule comprises SEQ ID NO: 2760, 2763, 2766, 2769, 2772, 2775, 2778, 2781, 2784, 2787, 2790, 2793, 2796, 2799, 2802, 2805, 2808, 2811, 2814, 2817, 2820, 2823, 2826, 2829, 2832, 2835, 2838, 2841, 2844, 2847, 2850, 2853, 2856, 2859, 2862, 2865, 2868, 2871, 2874, 2877, 2880, 2883, 2886, 3189, or 3195.

7. The system of claim 1, wherein the Cas protein is CcaCas13b and the guide molecule comprises SEQ ID NO: 2890, 2893, 2896, 2899, 2902, 2905, 2908, 2911, 2914, 2917, 2920, 2923, 2926, 2929, 2932, 2935, 2938, 2941, 2944, 2947, 2950, 2953, 2956, 2959, 2962, 2965, 2968, 2971, 2974, 2977, 2980, 2983, 2986, 2989, 2992, 2995, 2998, or 3001.

8. The system of claim 1, wherein the Cas protein is CcaCas13b and the guide molecule comprises SEQ ID NO: 2889, 2892, 2895, 2898, 2901, 2904, 2907, 2910, 2913, 2916, 2919, 2922, 2925, 2928, 2931, 2934, 2937, 2940, 2943, 2946, 2949, 2952, 2955, 2958, 2961, 2964, 2967, 2970, 2973, 2976, 2979, 2982, 2985, 2988, 2991, 2994, 2997, 3171, 3207, 3177 or 3213.

9. The system of claim 2, wherein the optimized guide is generated for a Cas13 ortholog.

10. The system of the claim 9, wherein the optimized guide is generated for an LwaCas13a or a CcaCas13b ortholog.

11. The system of claim 2, wherein the Cas protein is LwaCas13a and the guide molecule comprises a top predicted guide selected from SEQ ID NOs: 3153, 3159, 3189 or 3195 .

12. The system of claim 9, wherein the Cas protein is CcaCas13b and the guide molecule comprises a top predicted guide selected from SEQ ID NOs: 3207, 3231, 3171 or 3177.

13. The system of claim 1, wherein the guide molecule is directed to a BCR-ABL fusion.

14. The system of claim 13, wherein the BCR-ABL fusion is the BCR-ABL p210 b3a2 fusion, b2a2 fusion, or a p190 e1a2 fusion.

15. The system of claim 1, wherein the RNA-based masking construct comprises a silencing RNA that suppresses generation of a gene product encoded by a reporting construct, wherein the gene product generates a detectable positive signal when expressed.

16. The system of claim 1, wherein the RNA-based masking construct is a ribozyme that generates a negative detectable signal, and wherein the detectable positive signal is generated when the ribozyme is deactivated.

17. The system of claim 16, wherein the ribozyme converts a substrate to a first color and wherein the substrate converts to a second color when the ribozyme is deactivated.

18. The system of claim 1, wherein the RNA-based masking construct is an RNA aptamer and/or comprises an RNA-tethered inhibitor.

19. The system of claim 18, wherein the aptamer or RNA-tethered inhibitor sequesters an enzyme, wherein the enzyme generates a detectable signal upon release from the aptamer or RNA tethered inhibitor by acting upon a substrate.

20. The system of claim 18, wherein the aptamer is an inhibitory aptamer that inhibits an enzyme and prevents the enzyme from catalyzing generation of a detectable signal from a substrate or wherein the RNA-tethered inhibitor inhibits an enzyme and prevents the enzyme from catalyzing generation of a detectable signal from a substrate.

21. The system of claim 20, wherein the enzyme is thrombin, protein C, neutrophil elastase, subtilisin, horseradish peroxidase, beta-galactosidase, or calf alkaline phosphatase.

22. The system of claim 21, wherein the enzyme is thrombin and the substrate is para-nitroanilide covalently linked to a peptide substrate for thrombin, or 7-amino-4-methylcoumarin covalently linked to a peptide substrate for thrombin.

23. The system of claim 18, wherein the aptamer sequesters a pair of agents that when released from the aptamers combine to generate a detectable signal.

24. The system of claim 1, wherein the RNA-based masking construct comprises an RNA oligonucleotide to which a detectable ligand and a masking component are attached.

25. The system of claim 1, wherein the RNA-based masking construct comprises a nanoparticle held in aggregate by bridge molecules, wherein at least a portion of the bridge molecules comprises RNA, and wherein the solution undergoes a color shift when the nanoparticle is disbursed in solution.

26. The system of claim 25, wherein the nanoparticle is a colloidal metal, optionally colloidal gold.

27. The system of claim 1, wherein the detection construct is a gold nanoparticle, optionally modified with a binding agent that specifically binds the second molecule of the detection construct.

28. The system of claim 1, wherein the RNA-based masking construct comprising a quantum dot linked to one or more quencher molecules by a linking molecule, wherein at least a portion of the linking molecule comprises RNA.

29. The system of claim 1, wherein the RNA-based masking construct comprises RNA in complex with an intercalating agent, wherein the intercalating agent changes absorbance upon cleavage of the RNA.

30. The system of claim 29, wherein the intercalating agent is pyronine-Y or methylene blue.

31. The system of claim 1, wherein the detectable ligand is a fluorophore and the masking component is a quencher molecule.

32. The system of claim 1, wherein the RNA-based detection construct is a nucleic-acid based aptamer comprising quadruplex having enzymatic activity.

33. The system of claim 32, wherein the enzymatic activity is peroxidase activity.

34. The system of claim 1, wherein the detection construct comprises a first molecule on a first end and a second molecule on a second end.

35. The system of claim 34, wherein FAM is the first molecule and biotin or Digoxigenin (DIG) is the second molecule, or wherein Tye665 is the first molecule and Alexa-488 or FAM is the second molecule.

36. The system of claim 1, wherein the one or more Cas proteins is one or more Type V Cas proteins, one or more Type VI proteins, or a combination of Type V and Type VI proteins.

37. The system of claim 36, wherein the Type VI Cas protein is a Cas13.

38. The system of the claim 36, wherein the Type V Cas protein is a Cas12.

39. The system of claim 2, wherein the training model comprises one or more input features selected from guide sequence, flanking target sequence, normalized positions of the guide in the target and guide GC content.

40. The system of claim 39, wherein the guide sequence and/or flanking sequence input comprises one hit encoding mono-nucleotide and/or dinucleotide based identities across a guide length and flanking sequence in the target.

41. The system of claim 39, wherein the training model comprises applying logistic regression model on the activity of the guides across the one or more input features.

42. The system of claim 2, wherein the predicting highly active guides for the target molecule comprises selecting guides with an increase in activity of a guide relative to the median activity, or selecting guides with highest guide activity.

43. The system of claim 42, wherein the increase in activity is measured by an increase in fluorescence.

44. The system of claim 43, wherein the guides are selected with a 1.5, 2, 2.5 or 3-fold activity relative to median, or are in the top quartile or quintile for each target tested.

45. The system of any of the preceding claims, further comprising one or more amplification reagents to amplify the one or more target molecules.

46. The system of claim 45, wherein the reagents to amplify the one or more target RNA molecules comprise nucleic acid sequence-based amplification (NASBA), recombinase polymerase amplification (RPA), loop-mediated isothermal amplification (LAMP), strand displacement amplification (SDA), helicase-dependent amplification (HDA), nicking enzyme amplification reaction (NEAR), PCR, multiple displacement amplification (MDA), rolling circle amplification (RCA), ligase chain reaction (LCR), or ramification amplification method (RAM).

47. A lateral flow device comprising a substrate comprising a first end, wherein the first end comprises a sample loading portion and a first region loaded with a detection construct and one or more nucleic acid detection systems of any one of the preceding claims, a first capture region comprising a first binding agent, and a second capture region comprising a second binding agent.

48. The lateral flow device of claim 47, wherein the detection construct comprises a first molecule on a first end and a second molecule on a second end.

49. The lateral flow device of claim 48, comprising two nucleic acid detection systems and a first detection construct comprising Alexa-488 and a second detection construct comprising FAM.

50. The lateral flow device of claim 47, wherein the sample loading portion further comprises one or more amplification reagents to amplify the one or more target molecules.

51. A method for detecting a cancer fusion gene in a sample, comprising contacting the sample with the nucleic acid detection system of any of claims 1 to 46 or the lateral flow device of any one of claims 47-50; and detecting target fusion sequence.

52. The method of claim 51, further comprising amplifying the target molecules in the sample by RT-RPA.

53. The method of claim 51, wherein contacting the sample with the nucleic acid detection system comprises contacting the sample with a lateral flow device.

54. The method of claim 53, wherein the sample is blood, bone marrow, or pelleted cells.

55. The method of claim 53, further comprising steps of extracting RNA, performing RT-RPA, performing T7 transcription, and detecting the target nucleic acids.

56. The method of claim 53, wherein detecting the target nucleic acids comprises:
activating the Cas protein via binding of the one or more guide molecules to the one or more cancer-specific target molecules, wherein activating the Cas protein results in modification of the RNA-based masking construct such that a detectable positive signal is produced; and
detecting the signal, wherein detection of the signal indicates the presence of a cancer-specific fusion gene.

57. The method of claim 56, wherein detecting step is less than about 45 minutes to less than about 3 hours.

58. The method of claim 53, wherein a plurality of cancer fusion genes can be detected simultaneously on a multiplex lateral flow strip.

59. The method of claim 58, further comprising detecting PML-RARa Intron/exon 6 fusion and Intron 3 fusion simultaneously on multiplex lateral flow.

60. The method of claim 53, wherein the detection construct comprises a FAM and/or Alexa 488.

61. The method of claim 53, further comprising detected target fusion sequences with a sensitivity of about 2 fM, or about 200 aM.
